SEQUENCE LISTING (1 sequence)

SEQ ID NO: 1 - protein, 188 aa, Homo sapiens [sequence omitted]

(19) United States
(12) Patent Application Publication
(10) Pub. No.: US 2024/0084305 A1
(43) Pub. Date: Mar. 14, 2024

(54) MODULATORS OF CELL PROLIFERATION AND USES THEREOF

(71) Applicant: Kumquat Biosciences Inc., San Diego, CA (US)

(72) Inventors: Yi Liu, San Diego, CA (US); Matthew R. Janes, Carlsbad, CA (US); Rasmus Hansen, San Diego, CA (US); Pingda Ren, San Diego, CA (US); Karen K. Wong, Palo Alto, CA (US); Liansheng Li, San Diego, CA (US)

(21) Appl. No.: 18/297,211

(22) Filed: Apr. 7, 2023

Related U.S. Application Data
(63) Continuation-in-part of application No. PCT/US2021/054311, filed on Oct. 8, 2021.
(60) Provisional application No. 63/089,502, filed on Oct. 8, 2020.
(60) Provisional application No. 63/188,301, filed on May 13, 2021.

(51) Int. Cl. C12N 15/113; A61K 45/06; A61P 35/00

(52) U.S. Cl. CPC C12N 15/1135; A61K 45/06; A61P 35/00; C12N 2320/31; C12N 2320/10; C12N 2740/15043

(57) ABSTRACT

The present disclosure provides methods of inhibiting cell proliferation signaling in a cell. The present disclosure further provides compositions for inhibiting cell proliferation signaling in a cell. The methods and compounds have a range of utilities as therapeutics, diagnostics, and research tools.

### CROSS-REFERENCE

This application is a continuation-in-part of International Application No. PCT/US2021/054311, filed Oct. 8, 2021, which claims the benefit of U.S. Provisional Application No. 63/089,502, filed Oct. 8, 2020, and U.S. Provisional Application No. 63/188,301, filed May 13, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 7, 2023, is named 56690_715_501_SL.xml and is 1,888 bytes in size.

### BACKGROUND

Cancer is the second leading cause of death worldwide estimated to be responsible for about 10 million deaths each year. Many types of cancers are marked with mutations in one or more proteins involved in various signaling pathways leading to unregulated growth of cancerous cells. In some cases, about 25 to 30 percent (%) of tumors are known to harbor Rat sarcoma (Ras) mutations. Ras proteins such as human H-Ras, K-Ras, and N-Ras are small GTPase proteins involved in signal transduction pathways that regulate diverse cellular behaviors. When Ras proteins are activated or switched on by upstream signals, they in turn activate downstream components of signal transductions pathways, culminating in dysregulated cellular activities responsible for abnormal cell growth, differentiation, and/or survival.

K-Ras is one of the most frequently mutated oncogenes across a broad spectrum of human cancers, including lung cancer (e.g., non-small cell lung cancer), pancreatic, cervical, colorectal, stomach, uterine, skin, bladder, renal, breast, prostate, acute myeloid leukemia, ovarian, liver acute lymphoblastic leukemia, and brain cancers. Mutation and dysregulation of the function of N-Ras are associated with different lung cancers and melanoma. H-Ras mutations have been found associated with head and neck cancer and other types of cancer as well.

Ras proteins have long been considered to be “undruggable,” due to, in part, high affinity to their substrate Guanosine-5′-triphosphate (GTP) and/or their smooth surfaces without any obvious targeting region. Recently, a specific G12C Ras gene mutation has been identified as a druggable target. However, such a therapeutic approach is still limiting, as the G12C mutation in Ras has a low prevalence rate (e.g., about 3% in pancreatic ductal adenocarcinoma) as compared to other known Ras mutations including G12D, G12V, and G12S mutations.

Resistance to single Ras inhibitors may be problematic due to multiple potential resistance mechanisms such as secondary mutations in Ras and other mechanisms that could circumvent Ras inhibition including mutations in other components of other signaling pathways that cross-react with the Ras pathway. A potential strategy for overcoming or delaying the development of resistance to K-Ras or enhancing a sustained therapeutic efficacy is to combining K-Ras inhibitors with one or more additional therapeutic agents targeting additional cancer-associated genes or proteins. However, a vast number of signaling molecules across many different signaling pathways have bene implicated in various cancers. The therapeutic benefit of specific combinations targeting specific signaling molecules in different types of K-Ras driven cancers has largely not been explored.

### SUMMARY

There exists a considerable need for identifying relevant combination therapies to address the long-felt need for gaining sustained therapeutic benefit against various types of cancers. The present invention addresses this need and provides related advantages as well.

In view of the foregoing, there remains a considerable need for a new design of therapeutics and diagnostics that can specifically target intracellular or intracellular portions of cancer or other disease targets. There exists a considerable need for identifying relevant combination therapies to address the long-felt need for combination therapies using a plurality of agents to overcome various challenges or limitations associated with a single agent therapy, such as, potential resistance mechanism to the single agent, and/or undesired side effects associated with a therapeutically effective amount of the single agent. In particular, there remains a considerable need for compositions and methods applicable also for other Ras mutants and/or associated proteins of Ras to reduce Ras pathway signaling. Such compositions and methods can be particularly useful for treating a variety of diseases including, but not limited to, cancers and neoplasia conditions. The present disclosure addresses these needs, and provides additional advantages applicable for diagnosis, prognosis, and treatment for a wide diversity of diseases.

In one aspect, the disclosure provides a method of inhibiting cell proliferation signaling in a cell, comprising: downregulating, in the cell, expression or activity of: (a) Kras G12D and (b) one or more signaling molecules selected from Table 1.

In some embodiments, the downregulating is effectuated by contacting the cell with an inhibitor against (a) Kras G12D and an inhibitor against (b) the one or more signaling molecules selected from Table 1, wherein each inhibitor independently selected from the group consisting of a small molecule and a nucleic acid agent. In some embodiments, the nucleic acid agent comprises one or more members selected from the group consisting of an antisense oligonucleotide, a transfer RNA (tRNA), a small nuclear RNA (snRNA), a small interfering RNA (siRNA), a microRNA (miRNA), a small hairpin RNA (shRNA), a noncoding RNA (ncRNA), a pre-condensed DNA, an aptamer, a ribozyme, and a complex comprising a nucleic acid molecule and an endonuclease.

In some embodiments of any one of the preceding methods, the downregulating is effectuated by contacting the cell with an inhibitor against (a) Kras G12D and an inhibitor against (b) SOS, SHIP2, MEK, ERK, or EGFR. In some embodiments of any one of the preceding methods, the downregulating is effectuated by contacting the cell with an inhibitor against (a) Kras G12D and an inhibitor against (b) SOS, SHP2, MEK, ERK, or EGFR. In some embodiments of any one of the preceding methods, the downregulating is effectuated by contacting the cell with an inhibitor against (a) Kras G12D and an inhibitor against (b) SOS. In some embodiments of any one of the preceding methods, the downregulating is effectuated by contacting the cell with an inhibitor against (a) Kras G12D and two inhibitors against (b) two distinct signaling molecules selected from Table 1. In some embodiments of any one of the preceding methods, the downregulating is effectuated by contacting the cell with an inhibitor against (a) Kras G12D and two inhibitors against (b) three, four, or more distinct signaling molecules selected from Table 1, or any other distinct signaling molecule(s) disclosed herein. In some embodiments, the present disclosure provides a method comprising: administering (a) an inhibitor against a Ras G12D (e.g., KRas G12D) protein; and (b) an inhibitor against a Ras G12C (e.g., KRas G12C) protein. In some other embodiments, the present disclosure provides a method comprising: administering an inhibitor against (a) a Ras protein (e.g., a mutated Ras protein, such as Kras G12D), an inhibitor against (b) one or more signaling molecules selected from Table 1, and an inhibitor against (c) a Ras G12C (e.g., KRas G12C) protein.

In some embodiments of any one of the preceding methods, the contacting with the inhibitor against (a) and the inhibitor(s) against (b) occurs concurrently. In some embodiments of any one of the preceding methods, the contacting with the inhibitor against (a) occurs before or after the contacting with inhibitor(s) against (b).

In some embodiments of any one of the preceding methods, the contacting occurs in vitro, ex vivo, or in vivo.

In some embodiments of any one of the preceding methods, the method further comprises co-administering, to a subject comprising the cell, an inhibitor against (a) and an inhibitor(s) against (b). In some embodiments, the co-administering comprises administering the inhibitor against (a) and the inhibitor(s) against (b) concurrently. In some embodiments, the co-administering comprises administering the inhibitor against (a) before or after administering the inhibitor(s) against (b).

In some embodiments of any one of the preceding methods, the inhibitor against (a) and/or the inhibitor(s) against (b) is administered parenterally, orally, intraperitoneally, intravenously, intraarterially, transdermally, intramuscularly, liposomally, via local delivery by catheter or stent, subcutaneously, intraadiposally, or intrathecally. In some embodiments, the inhibitor against (a) and the inhibitor(s) against (b) are administered in a same formulation. In some embodiments, the inhibitor against (a) and the inhibitor(s) against (b) are administered in different formulations.

In some embodiments of any one of the preceding methods, the inhibitor against (a) and the inhibitor(s) against (b) are parts of a single compound. In some embodiments, the single compound has a molecular weight of greater than 800 Dalton. In some embodiments, the inhibitor against (a) and the inhibitor(s) against (b) are coupled to one another via a linker moiety. In some embodiments, the linker moiety comprises 1 to 50 non-hydrogen atoms. In some embodiments, the linker moiety comprises one or more groups, in a branched or linear configuration, independently selected from alkyl, alkenyl, alkynyl, alkoxy, aryl, heteroaryl, heterocyclylalkyl, cycloalkyl, O, S, N, halo, hydroxyl, amino, cyano, and oxo.

In some embodiments of any one of the preceding methods, the inhibitor is capable of specifically binding to (a) or a gene encoding (a). In some embodiments of any one of the preceding methods, the inhibitor is capable of specifically binding to aspartic acid 12 residue of Kras. In some embodiments of any one of the preceding methods, the inhibitor is capable of specifically binding to (b) or one or more genes encoding (b). In some embodiments of any one of the preceding methods, the downregulating the expression or activity of (a) and that of (b) synergistically yields a greater degree of inhibition of cell proliferation in the cell as compared to (1) an individual degree of inhibition of cell proliferation via downregulating expression or activity of one of: (a) and (b), alone, and/or (2) a sum of the individual degrees of inhibition of cell proliferation thereof. In some embodiments of any one of the preceding methods, (b) comprises two or more signaling molecules selected from Table 1. In some embodiments of any one of the preceding methods, (b) comprises one or more members selected from the group consisting of (i) SOS1 or a mutant thereof, (ii) SHP2 or a mutant thereof, (iii) MEK or a mutant thereof, (iv) ERK or a mutant thereof, and (v) EGFR or a mutant thereof.

In some embodiments of any one of the preceding methods, the method further comprises contacting the cell with one or more pharmacologically active substances selected from Table 2.

In some embodiments of any one of the preceding methods, the cell can be part of cancer cells. In some embodiments of any one of the preceding methods, the cancer cells are in or derived from a subject in need thereof. In some embodiments of any one of the preceding methods, contacting the cancer cells with the inhibitor against (a) and the inhibitor against (b) exhibits a synergistic effect in reducing proliferation of the cancer cells. In some embodiments of any one of the preceding methods, contacting the cancer cells with the inhibitor against (a) and the inhibitor against (b) effects reduced proliferation of the cancer cells by at least about 20%, 40%, 50%, 60%, 70%, 80%, 90%, 95%, 99%, or more. In some embodiments of any one of the preceding methods, administering to the subject the inhibitor against (a) and the inhibitor against (b) exhibits a synergistic effect in reducing proliferation of the cancer cells in the subject. In some embodiments of any one of the preceding methods, administering to the subject the inhibitor against (a) and the inhibitor against (b) effects reduced proliferation of the cancer cells in the subject by at least about 20%, 40%, 50%, 60%, 70%, 80%, 90%, 95%, 99%, or more.

In some embodiments of any one of the preceding methods, the step of downregulating comprises contacting the cancer cells with the inhibitor against (a) and the inhibitor against (b), wherein contacting the cancer cells with the inhibitor against (a) occurs prior to, concurrently with, or subsequent to contacting the cancer cells with the inhibitor against (b), to effect a reduced proliferation of the cancer cells, wherein the reduced proliferation of the cancer cells by application of the inhibitor against (a) and the inhibitor against (b) is characterized by a synergistic value of at least about 0.05 as ascertained by Bliss independent criterion. In some embodiments, the synergistic value is ascertained by Bliss independent criterion in accordance with the formula:

Y_{AB,O}−Y_{AB,P}
wherein:
Y_{AB,O} is observed percentage growth inhibition of the cancer cells by the application of the inhibitor against (a) and the inhibitor against (b), comprising the inhibitor against (a) at dose A and the inhibitor against (b) at dose B; and
Y_{AB,P} is predicted percentage growth inhibition of the cancer cells by the application of the inhibitor against (a) and the inhibitor against (b), comprising the inhibitor against (a) at the dose A, and the inhibitor against (b) at the dose B, wherein Y_{AB,P}=Y_{A}+Y_{B}−Y_{A}Y_{B},
   wherein further:
   Y_{A} is observed percentage growth inhibition of the cancer cells by the inhibitor against (a) alone at the dose A;
   Y_{B} is observed percentage growth inhibition of the cancer cells by the inhibitor against (b) alone at the dose B; and
   Y_{A}Y_{B} is product of Y_{A} and Y_{B}.

In some embodiments, the synergistic value is at least about 0.05, 0.06, 0.07, 0.08, 0.09, or 0.1. In some embodiments, the synergistic value is at least about 0.2 or higher.

In some embodiments of any one of the preceding methods, the step of downregulating comprises contacting the cancer cells with the inhibitor against (a) and the inhibitor against (b) the signaling molecule selected from the group consisting of SOS, SHP2, and EGFR, wherein the contacting the cancer cells with the inhibitor against (a) occurs prior to, concurrently with, or subsequent to contacting the cancer cells with the inhibitor against (b), to effect a reduced proliferation of the cancer cells in the subject.

In some embodiments of any one of the preceding methods, the subject exhibits a genetic aberration in a PI3K gene, and the method comprises administering to the subject the inhibitor against (a) the Kras G12D and the inhibitor against (b) PI3K, wherein the inhibitor against (a) is administered prior to, concurrently with, or subsequent to administering the inhibitor against (b), such that application of the inhibitor against (a) and the inhibitor against (b) effects reduced proliferation of colorectal cancer cells or gastric cancer cells in the subject.

In some embodiments of any one of the preceding methods, the method comprises (a) assessing for presence of a genetic aberration in a PI3K gene in a biological sample comprising nucleic acid molecules derived from the subject, and (2) upon detecting the presence of the genetic aberration in the PI3K gene (e.g., PI3Ka or also denoted as PI3K-alpha), administering to the subject the inhibitor against (a) the Kras G12D inhibitor and the inhibitor against (b) PI3K, wherein the inhibitor against (a) is administered prior to, concurrently with, or subsequent to administering the inhibitor against (b), such that application of the inhibitor against (a) and the inhibitor against (b) effects reduced proliferation of colorectal cancer cells or gastric cancer cells in the subject.

In some embodiments of any one of the preceding methods, the administering to the subject the inhibitor against (a) Kras G12D and the inhibitor against (b) PI3K (e.g., PI3Ka or also denoted as PI3K-alpha), effects reduced proliferation of the colorectal cancer cells in the subject by at least about 20%, 40%, 50%, 60%, 70%, 80%, 90%, 95%, 99%, or more. In some embodiments of any one of the preceding methods, the administering to the subject the inhibitor against (a) Kras G12D and the inhibitor against (b) PI3K effects reduced proliferation of the gastric cancer cells in the subject by at least about 20%, 40%, 50%, 60%, 70%, 80%, 90%, 95%, 99%, or more.

In some embodiments of any one of the preceding methods, the subject exhibits a genetic aberration in a CDK4/6 gene, and the method comprises administering to the subject the inhibitor against (a) the Kras G12D and the inhibitor against (b) CDK4/6, wherein the inhibitor against (a) is administered prior to, concurrently with, or subsequent to administering the inhibitor against (b), such that application of the inhibitor against (a) and the inhibitor against (b) effects reduced proliferation of non-small cell lung cancer cells or colorectal cancer cells in the subject.

In some embodiments of any one of the preceding methods, the method comprises (a) assessing for presence of a genetic aberration in a CDK4/6 gene in a biological sample comprising nucleic acid molecules derived from the subject, and (2) upon detecting the presence of the genetic aberration in the CDK4/6 gene, administering to the subject the inhibitor against (a) the Kras G12D inhibitor and the inhibitor against (b) CDK4/6, wherein the inhibitor against (a) is administered prior to, concurrently with, or subsequent to administering the inhibitor against (b), such that application of the inhibitor against (a) and the inhibitor against (b) effects reduced proliferation of non-small cell lung cancer cells or colorectal cancer cells in the subject.

In some embodiments of any one of the preceding methods, the administering to the subject the inhibitor against (a) Kras G12D and the inhibitor against (b) CDK4/6 effects reduced proliferation of the non-small cell lung cancer cells in the subject by at least about 20%, 40%, 50%, 60%, 70%, 80%, 90%, 95%, 99%, or more. In some embodiments of any one of the preceding methods, the administering to the subject the inhibitor against (a) Kras G12D and the inhibitor against (b) CDK4/6 effects reduced proliferation of the colorectal cancer cells in the subject by at least about 20%, 40%, 50%, 60%, 70%, 80%, 90%, 95%, 99%, or more.

In some embodiments of any one of the preceding methods, the method comprises administering to the cancer cells (a) a Kras G12D inhibitor exhibiting a cellular IC50 value of less than about 5 micromolar (μM) against the cancer cells, and (b) at least one inhibitor of the signaling molecule selected from the group consisting of SOS, SHP2, EGFR, and PI3K, wherein application of (a) and (b) yields a comparable degree of growth inhibition of the cancer cells to that mediated by a control Kras G12D inhibitor alone, wherein the control is a more potent Kras G12D inhibitor than (a), exhibiting a cellular IC50 value that is at least about one order of magnitude less than that of (a), wherein the cellular IC50 value of (a) and the cellular IC50 value of the control are ascertained by an in vitro growth inhibition assay utilizing the cancer cells.

In some embodiments of any one of the preceding methods, the cellular IC50 value of (a) is less than about 1 μM but greater than 10 nanomolar (nM). In some embodiments of any one of the preceding methods, the cellular IC50 value of (a) is less than about 0.5 μM but greater than 50 nM. In some embodiments of any one of the preceding methods, the cellular IC50 value of (a) is less than about 0.5 μM but greater than 100 nM. In some embodiments of any one of the preceding methods, (a) exhibits a cellular IC50 value greater than 10 nM, and the control exhibits a cellular IC50 value less than 1 nM. In some embodiments of any one of the preceding methods, the cellular IC50 value of the control is at least about one, two, three, four or more orders of magnitude less than that of (a).

In some embodiments of any one of the preceding methods, the method comprises administering to the cancer cells (a) a Kras G12D inhibitor and (b) at least one inhibitor of the signaling molecule selected from the group consisting of SOS, SHP2, EGFR, and PI3K, wherein a degree of in vitro growth inhibition of the cancer cells by application of (a) and (b) is comparable to that by either: (1) administering to the cancer cells the Kras G12D inhibitor alone in an amount greater than that used in the combination by at least about 2-fold, or (2) administering to the cancer cells the at least one inhibitor alone in an amount greater than that used in the combination by at least about 2-fold.

In some embodiments of any one of the preceding methods, the Kras G12D inhibitor alone is administered in an amount greater than that used in the application of (a) and (b) by at least about 4-fold, in (1). In some embodiments of any one of the preceding methods, the Kras G12D inhibitor alone is administered in an amount greater than that used in the application of (a) and (b) by at least about 5-fold, in (1). In some embodiments of any one of the preceding methods, the Kras G12D inhibitor alone is administered in an amount greater than that used in the application of (a) and (b) by at least about 10-fold, in (1). In some embodiments of any one of the preceding methods, the Kras G12D inhibitor alone is administered in an amount greater than that used in the application of (a) and (b) by at least about 20-fold, in (1). In some embodiments of any one of the preceding methods, the Kras G12D inhibitor alone is administered in an amount greater than that used in the application of (a) and (b) by at least about 5-fold, in (2).

In some embodiments of any one of the preceding methods, the cancer cells are derived from one or more members selected from the group consisting of non-small cell lung cancer, pancreatic cancer, colorectal cancer, gastric cancer, and endometrial cancer. In some embodiments of any one of the preceding methods, the cancer cells are derived from colorectal cancer or gastric cancer.

In some embodiments of any one of the preceding methods, the signaling molecule is selected from the group consisting of SOS, SHP2, EGFR, MEK, CDK4/6, and PI3Ka. In some embodiments of any one of the preceding methods, the signaling molecule is PI3K. In some embodiments of any one of the preceding methods, the signaling molecule is CDK4/6. In some embodiments of any one of the preceding methods, the signaling molecule is SHP2. In some embodiments of any one of the preceding methods, the signaling molecule is SOS. In some embodiments of any one of the preceding methods, the signaling molecule is EGFR

In some embodiments of any one of the preceding methods, the cancer cells are contacted with at least two inhibitors, one of which is an inhibitor of SOS, and another is an inhibitor of EGFR

In some embodiments of any one of the preceding methods, the signaling molecule is EGFR, and wherein the step of downregulating comprises contacting the cancer cells with Erlotinib or Afatinib.

In some embodiments of any one of the preceding methods, the downregulating reduces Ras signaling output in a cell. In some embodiments, the reduction in Ras signaling output is evidenced by one or more members selected from the group consisting of (i) an increase in steady state level of GDP-bound Ras protein; (ii) a reduction of phosphorylated AKT^{s473}, (iii) a reduction of phosphorylated ERK^{T202/y204}, (iv) a reduction of phosphorylated S6^{S235/236}, and (v) inhibition of cell growth of the cell.

In some embodiments of any one of the preceding methods, the cell is a tumor cell. In some embodiments, the tumor cell comprises a Ras-driven tumor cell selected from the group consisting of A549, AGS, ASPC1, Calu-6, CFPAC-1, CL40, COLO678, COR-L23, DAN-G, GP2D, GSU, HCT116, HEClA, HEC1B, HEC50B, HEYA8, HPAC, HPAFII, HUCCT1, KARPAS620, KOPN8, KP-3, KP-4, L3.3, LoVo, LS180, LS513, MCAS, NCI-H1355, NCI-H1573, NCI-H1944, NCI-H2009, NCI-H441, NCI-H747, OV7, PANC0203, PANC0403, PANC0504, PANC0813, PANC1, Panc-10.05, PaTu-8902, PK1, PK45H, PK59, SK-CO-1, SKLU1, SNU1, SNU1033, SNU1197, SNU407, SNU410, SNU601, SNU61, SNU8, SNU869, SNU-C2A, SU.86.86, SUIT2, SW1990, SW403, SW480, SW620, SW948, T3M10, TCC-PAN2, TGBC11TKB, and MIA Pa-Ca cell.

In some embodiments of any one of the preceding methods, the method is for treating a proliferative disorder. In some embodiments, the proliferative disorder is a neoplastic condition comprising a solid tumor.

In some embodiments of any one of the preceding methods, the method is for treating a disease condition associated with Kras G12D mutation.

In embodiments of the method, the Kras G12D inhibitor has a formula selected from formulae CA to CE (i.e., CA, CB, CC, CB, CD, CE), as disclosed herein, and the at least one inhibitor has a formula selected from formulae CF to CZ, DA to DZ, A to F, N to Z, Z′, AA, AA′, BB, BC, and BC′.

In embodiments of the method, the Kras G12D inhibitor has a formula selected from formulae CA to CE, and CF′ to CJ′, as disclosed herein, and the at least one inhibitor has a formula selected from formulae CF to CZ, DA to DZ, A to F, N to Z, Z′, AA, AA′, BB, BC, BC′, CK′.

In embodiments of the method, the Kras G12D inhibitor having a formula selected from formulae CA to CE can be used in combination or in conjunction with an inhibitor of EGFR is selected from afatinib, erlotinib, gefitinib, lapatinib, cetuximab panitumumab, osimertinib, olmutinib, and EGF-816. In embodiments of the method, any of the aforementioned Kras G12D inhibitor can be used in combination or in conjunction with an inhibitor of CDK4/6 is selected from palbociclib, ribociclib, and abemaciclib. In embodiments of the method, any of the aforementioned Kras G12D inhibitor can be used in combination or in conjunction with an inhibitor of MEK. In embodiments of the method, any of the aforementioned Kras G12D inhibitor can be used in combination or in conjunction with an inhibitor of PI3K alpha.

In embodiments of the method, the Kras G12D inhibitor has a formula selected from formulae CA to CE, and the at least one inhibitor has a formula selected from formulae BB, BB′, BC′, CF, CG, CH, CI, CJ, CK, CK′.

In embodiments of the method, the Kras G12D inhibitor has a formula selected from formulae CA to CE and the at least one inhibitor has a formula selected from formulae CL-CZ, DA-DZ.

In embodiments of the method, the Kras G12D inhibitor has a formula selected from formulae CF′ to CJ′ and an EGFR inhibitor is selected from afatinib, erlotinib, gefitinib, lapatinib, cetuximab panitumumab, osimertinib, olmutinib, and EGF-816. In embodiments of the method, any of the aforementioned Kras G12D inhibitor can be used in combination or in conjunction with an inhibitor of CDK4/6 selected from palbociclib, ribociclib, and abemaciclib. In embodiments of the method, any of the aforementioned Kras G12D inhibitor can be used in combination or in conjunction with an inhibitor of MEK. In embodiments of the method, any of the aforementioned Kras G12D inhibitor can be used in combination or in conjunction with an inhibitor of PI3K alpha.

In embodiments of the method, the Kras G12D inhibitor has a formula selected from formulae CF′ to CJ′ can be used in combination or in conjunction with an inhibitor of formula selected from BB, BB′, BC′, CF, CG, CH, CI, CJ, CK, CK′.

In embodiments of the method, the Kras G12D inhibitor having a formula selected from formulae CF′ to CJ′ can be used in combination or in conjunction with an inhibitor of formula selected from CL-CZ, DA-DZ.

In some embodiments of any one of the preceding methods, the Kras G12D inhibitor has the formula (CA), as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CA), as disclosed herein, and the at least one inhibitor has a formula selected from formulae CL to CZ and DA to DZ. In embodiments of the subject method, the Kras G12D inhibitor has a formula selected from formulae CF′ to CJ′, as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has a formula selected from formulae CF′ to CJ′, as disclosed herein, and the at least one inhibitor has a formula selected from BB, BC, BC′, CF, CG, CH, CI, CJ, CK, and CK′. In embodiments of the subject method, the Kras G12D inhibitor has a formula selected from formulae CF′ to CJ′, as disclosed herein, and the at least one inhibitor has a formula selected from CL to CZ and DA to DZ. In embodiments of the subject method, the combination comprises (a) a Kras G12D inhibitor having a formula selected from formulae CF′ to CJ′, as disclosed herein, and an inhibitor of EGFR selected from afatinib, erlotinib, gefitinib, lapatinib, cetuximab panitumumab, osimertinib, olmutinib, and EGF-816. In embodiments of the subject method, the Kras G12D inhibitor has a formula selected from formulae CF′ to CJ′, as disclosed herein, and the inhibitor of CDK4/6 is selected from palbociclib, ribociclib, and abemaciclib. In embodiments of the subject method, the Kras G12D inhibitor has the formula (CF′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CF′), as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CF′), as disclosed herein, and the at least one inhibitor has a formula selected from formulae CL to CZ and DA to DZ. In embodiments of the subject method, the Kras G12D inhibitor has the formula (CF′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has the formula (CF′), as disclosed herein, and the inhibitor of CDK4/6 is selected from palbociclib, ribociclib, and abemaciclib. In embodiments of the subject method, the Kras G12D inhibitor has the formula (CF′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CG′) or formula (CH′), as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CG′) or formula (CH′), as disclosed herein, and the at least one inhibitor has a formula selected from formulae CL to CZ and DA to DZ. In embodiments of the subject method, the Kras G12D inhibitor has the formula (CG′) or formula (CH′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has formula (CG′) or formula (CH′), as disclosed herein, and the inhibitor of CDK4/6 is selected from palbociclib, ribociclib, and abemaciclib. In embodiments of the subject method, the Kras G12D inhibitor has the formula (CI′) as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CI′), as disclosed herein, and the at least one inhibitor has a formula selected from formulae CL to CZ and DA to DZ. In embodiments of the subject method, the Kras G12D inhibitor has the formula (CI′), as disclosed herein, and the inhibitor of EGFR is selected from afatinib, erlotinib, gefitinib, lapatinib, cetuximab panitumumab, osimertinib, olnutinib, and EGF-816. In embodiments of the subject method, the Kras G12D inhibitor has formula (CI′), as disclosed herein, and the inhibitor of CDK4/6 is selected from palbociclib, ribociclib, and abemaciclib. In embodiments of the subject method, the Kras G12D inhibitor has the formula (CJ′) as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CJ′), as disclosed herein, and the at least one inhibitor has a formula selected from formulae CL to CZ and DA to DZ. In embodiments of the subject method, the Kras G12D inhibitor has the formula (CJ′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has formula (CJ′), as disclosed herein, and the inhibitor of CDK4/6 is selected from palbociclib, ribociclib, and abemaciclib.

In another aspect, the disclosure provides a modified cell characterized by exhibiting downregulated expression or activity of (a) and that of (b) in accordance with the method of any one of the preceding claims.

In yet another aspect, the disclosure provides a modified cell in which expression or activity of: (a) Kras G12D and (b) one or more signaling molecules selected from Table 1 is downregulated by an inhibitor against (a) and an inhibitor against (b).

In some embodiments of any one of the preceding modified cells, the modified cell is characterized by exhibiting reduced Ras signaling output as compared to a control cell subjected to one of: the inhibitor against (a) and inhibitor against (b), alone. In some embodiments, the reduction in Ras signaling output is evidenced by one or more members selected from the group consisting of (i) an increase in steady state level of GDP-bound Ras protein; (ii) a reduction of phosphorylated AKT^{s73}, (iii) a reduction of phosphorylated ERK^{T202/y204}, (iv) a reduction of phosphorylated S6^{S235/236}, and (v) inhibition of cell growth of the modified cell.

In some embodiments of any one of the preceding modified cells, the modified cell comprises the inhibitor against (a) and the inhibitor against (b).

In some embodiments of any one of the preceding modified cells, the inhibitor against (a) or the inhibitor against (b) is selected from the group consisting of a small molecule and a nucleic acid agent. In some embodiments, the nucleic acid agent comprises one or more members selected from the group consisting of an antisense oligonucleotide, a transfer RNA (tRNA), a small nuclear RNA (snRNA), a small interfering RNA (siRNA), a microRNA (miRNA), a small hairpin RNA (shRNA), a noncoding RNA (ncRNA), a pre-condensed DNA, an aptamer, a ribozyme, and a complex comprising a nucleic acid molecule and an endonuclease. In some embodiments, expression or activity of (a) Kras G12D and that of (b) SOS, SHIP2, MEK, ERK, or EGFR is inhibited. In some embodiments, expression or activity of (a) Kras G12D and that of (b) SOS, SHP2, MEK, ERK, or EGFR is inhibited. In some embodiments, expression or activity of (a) Kras G12D and that of (b) SOS is inhibited.

In some embodiments of any one of the preceding modified cells, the modified cell is a tumor cell. In some embodiments, the tumor cell comprises a Ras-driven tumor cell selected from the group consisting of A549, AGS, ASPC1, Calu-6, CFPAC-1, CL40, COLO678, COR-L23, DAN-G, GP2D, GSU, HCT116, HEClA, HEC1B, HEC50B, HEYA8, HPAC, HPAFII, HUCCT1, KARPAS620, KOPN8, KP-3, KP-4, L3.3, LoVo, LS180, LS513, MCAS, NCI-H1355, NCI-H1573, NCI-H1944, NCI-H2009, NCI-H441, NCI-H747, OV7, PANC0203, PANC0403, PANC0504, PANC0813, PANC1, Panc-10.05, PaTu-8902, PK1, PK45H, PK59, SK-CO-1, SKLU1, SNU1, SNU1033, SNU1197, SNU407, SNU410, SNU601, SNU61, SNU8, SNU869, SNU-C2A, SU.86.86, SUIT2, SW1990, SW403, SW480, SW620, SW948, T3M10, TCC-PAN2, TGBC11TKB, and MIA Pa-Ca cell.

In some embodiments of any one of the preceding modified cells, the expression or activity of (a) and that of (b) is further downregulated by one or more pharmacologically active substances selected from Table 2.

In another aspect, the disclosure provides a composition comprising: an inhibitor against (a) Kras G12D; and an inhibitor against (b) one or more signaling molecules selected from Table 1. Also provided in the present disclosure is a composition comprising (a) an inhibitor against a Ras G12D (e.g., KRas G12D) protein; and (b) an inhibitor against a Ras G12C (e.g., KRas G12C) protein. Yet further provided in the present disclosure is a composition comprising: an inhibitor against (a) a Ras protein (e.g., a mutated Ras protein, such as Kras G12D), an inhibitor against (b) one or more signaling molecules selected from Table 1, and an inhibitor against (c) a Ras G12C (e.g., KRas G12C) protein.

In some embodiments, the inhibitor against (b) specifically inhibits one of the signaling molecules selected from Table 1.

In some embodiments of any one of the preceding compositions, the inhibitor against (a) or the inhibitor against (b) is selected from the group consisting of a small molecule and a nucleic acid agent. In some embodiments, the nucleic acid agent comprises one or more members selected from the group consisting of an antisense oligonucleotide, a transfer RNA (tRNA), a small nuclear RNA (snRNA), a small interfering RNA (siRNA), a microRNA (miRNA), a small hairpin RNA (shRNA), a noncoding RNA (ncRNA), a pre-condensed DNA, an aptamer, a ribozyme, and a complex comprising a nucleic acid molecule and an endonuclease.

In some embodiments of any one of the preceding compositions, the composition comprises an inhibitor against (a) KrasG12D and one or more inhibitor against (b) SOS, SHIP2, MEK, ERK, or EGFR In some embodiments of any one of the preceding compositions, the composition comprises an inhibitor against (a) KrasG12D and one or more inhibitor against (b) SOS, SHP2, MEK, ERK, or EGFR In some embodiments of any one of the preceding compositions, the composition comprises an inhibitor against (a) KrasG12D and an inhibitor against (b) SOS. In some embodiments of any one of the preceding compositions, the composition comprises an inhibitor against (a) KrasG12D, (b) an inhibitor against SOS, and (c) an inhibitor against EGFR

In some embodiments of any one of the preceding compositions, the inhibitor against (a) or the inhibitor against (b) is formulated for administration selected from the group consisting of parenteral administration, oral administration, intraperitoneal administration, intravenous administration, intraarterial administration, transdermal administration, intramuscular administration, liposomal administration, local delivery by catheter or stent, subcutaneous administration, intraadiposal administration, and intrathecal administration. In some embodiments, the inhibitor against (a) and the inhibitor against (b) are administered in a same formulation.

In some embodiments, the inhibitor against (a) and the inhibitor against (b) are administered in different formulations.

In some embodiments of any one of the preceding compositions, an inhibitor against (a) and an inhibitor against (b) are parts of a single compound. In some embodiments, the single compound has a molecular weight of greater than or less than 800 Dalton. In some embodiments, the inhibitor against (a) and the inhibitor against (b) are coupled to one another via a linker moiety. In some embodiments, the linker moiety comprises 1 to 50 non-hydrogen atoms. In some embodiments, the linker moiety comprises one or more groups, in a branched or linear configuration, independently selected from alkyl, alkenyl, alkynyl, alkoxy, aryl, heteroaryl, heterocyclylalkyl, cycloalkyl, O, S, N, halo, hydroxyl, amino, cyano, and oxo.

In some embodiments of any one of the preceding compositions, the inhibitor against (a) is capable of downregulating expression or activity of (a). In some embodiments of any one of the preceding compositions, the inhibitor against (b) is capable of downregulating expression or activity of (b). In some embodiments of any one of the preceding compositions, the inhibitor against (a) is capable of specifically binding to (a) or a gene encoding (a). In some embodiments of any one of the preceding compositions, the inhibitor against (a) is capable of specifically binding to aspartic acid 12 residue of Kras. In some embodiments of any one of the preceding compositions, the inhibitor against (b) is capable of specifically binding to (b) or one or more genes encoding (b).

In some embodiments of any one of the preceding compositions, a combination of the inhibitor against (a) and the inhibitor against (b) synergistically reduces Ras signaling output in a cell as compared to one of: the inhibitor of (a) and the inhibitor of (b), alone. In some embodiments, the reduction in Ras signaling output is evidenced by one or more members selected from the group consisting of (i) an increase in steady state level of GDP-bound Ras protein; (ii) a reduction of phosphorylated AKT^{s473}, (iii) a reduction of phosphorylated ERK^{T202/y204}, (iv) a reduction of phosphorylated S6^{S235/236}, and (v) inhibition of cell growth of the cell.

In some embodiments of any one of the preceding compositions, the cell is a tumor cell. In some embodiments, the tumor cell comprises a Ras-driven tumor cell selected from the group consisting of A549, AGS, ASPC1, Calu-6, CFPAC-1, CL40, COLO678, COR-L23, DAN-G, GP2D, GSU, HCT116, HEClA, HEC1B, HEC50B, HEYA8, HPAC, HPAFII, HUCCT1, KARPAS620, KOPN8, KP-3, KP-4, L3.3, LoVo, LS180, LS513, MCAS, NCI-H1355, NCI-H1573, NCI-H1944, NCI-H2009, NCI-H441, NCI-H747, OV7, PANC0203, PANC0403, PANC0504, PANC0813, PANC1, Panc-10.05, PaTu-8902, PK1, PK45H, PK59, SK-CO-1, SKLU1, SNU1, SNU1033, SNU1197, SNU407, SNU410, SNU601, SNU61, SNU8, SNU869, SNU-C2A, SU.86.86, SUIT2, SW1990, SW403, SW480, SW620, SW948, T3M10, TCC-PAN2, TGBC11TKB, and MIA Pa-Ca cell.

In some embodiments of any one of the preceding compositions, a combination of the inhibitor against (a) and the inhibitor against (b) provides a synergistic therapeutic effect in a subject in need thereof as compared to one of: the inhibitor against (a) and the inhibitor against (b), alone.

In some embodiments of any one of the preceding compositions, the composition is for use in reducing proliferation of cancer cells and/or treating cancer in a subject in need thereof. In some embodiments of any one of the preceding compositions, the composition is a pharmaceutical composition for treating a proliferative disorder. In some embodiments, the proliferative disorder is a neoplastic condition selected from the group consisting of lung cancer, head and neck squamous cell carcinoma, pancreatic cancer, breast cancer, ovarian cancer, Kaposi's sarcoma, renal cell carcinoma, prostate cancer, neuroendocrine cancer, and endometrial cancer.

In some embodiments of any one of the preceding compositions, the composition is a pharmaceutical composition for treating a disease condition associated with Kras G12D mutation.

In some embodiments of any one of the preceding compositions, (b) comprises two or more signaling molecules selected from Table 1. In some embodiments of any one of the preceding compositions, (b) comprises one or more members selected from the group consisting of (i) SOS1 or a mutant thereof, (ii) SHP2 or a mutant thereof, (iii) MEK or a mutant thereof, and (iv) ERK or a mutant thereof, and (v) EGFR or a mutant thereof.

In some embodiments of any one of the preceding compositions, the composition further comprises one or more pharmacologically active substances selected from Table 2.

In a different aspect, the disclosure provides a kit comprising: the composition of any one of the preceding claims; and instructions directing (i) contacting a cell with the composition or (ii) administration of the composition to a subject in need thereof.

In some embodiments, the contacting occurs in vitro, ex vivo, or in vivo.

In some embodiments, the composition of the kit comprises the Kras G12D inhibitor and the at least one inhibitor. In some embodiments, the Kras G12D inhibitor and the at least one inhibitor are in a same unit dosage. In some embodiments, the Kras G12D inhibitor and the at least one inhibitor are in different unit dosages.

In an embodiment, the subject method comprises administering a Kras G12D inhibitor having a formula selected from formulae CA to CE, as disclosed herein, in combination with an inhibitor of one or more signaling molecules selected from Table 1 having a formula selected from formulae CF to CZ, DA to DZ, A to F, N to Z, Z′, AA, AA′, BB, BC, and BC′. In an embodiment, the subject method comprises administering a Kras G12D inhibitor having a formula selected from formulae CA to CE, as disclosed herein, in conjunction with an inhibitor of one or more signaling molecules selected from Table 1 having a formula selected from formulae CF to CZ, DA to DZ, A to F, N to Z, Z′, AA, AA′, BB, BC, and BC′. In an embodiment, the subject method comprises administering a Kras G12D inhibitor having a formula selected from formulae CA to CE, as disclosed herein, in combination with an inhibitor of SHP2 selected from

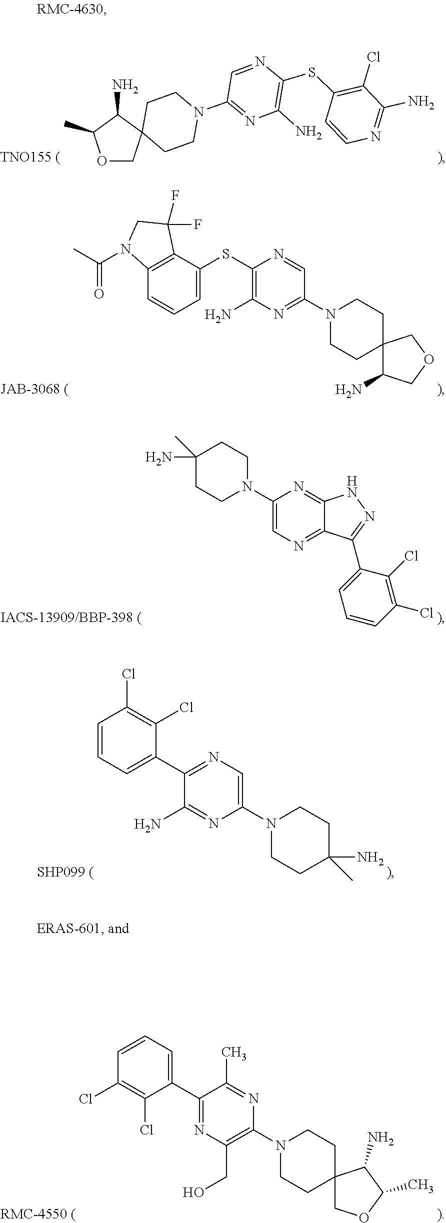 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In an embodiment, the subject method comprises administering a Kras G12D inhibitor having a formula selected from formulae CA to CE, as disclosed herein, in conjunction with an inhibitor of SHP2 selected from

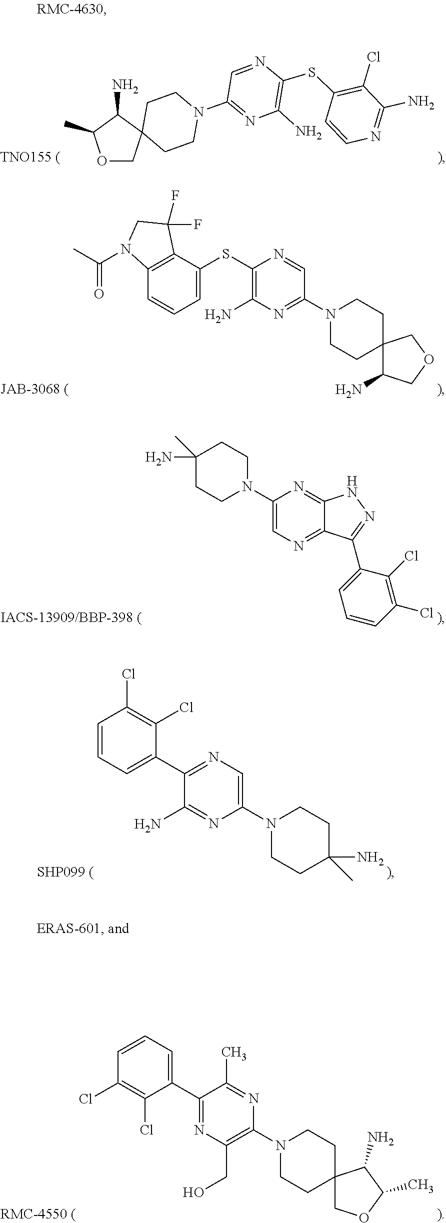 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In an embodiment, the subject method comprises administering a Kras G12D inhibitor having a formula selected from formulae CA to CE, as disclosed herein, in combination with an inhibitor of SOS selected from

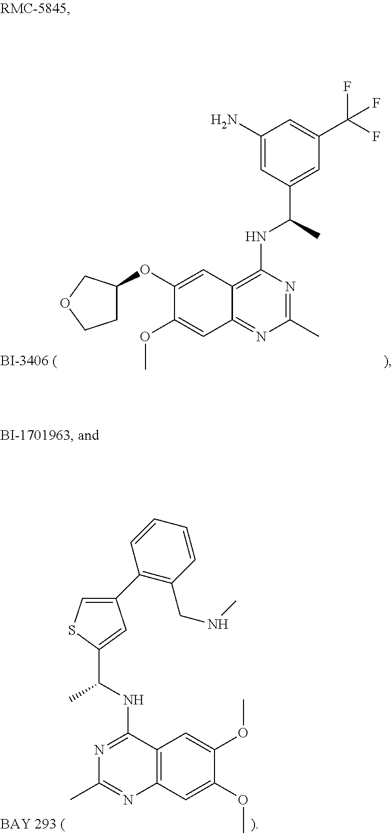 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1]

In an embodiment, the subject method comprises administering a Kras G12D inhibitor having a formula selected from formulae CA to CE, as disclosed herein, in conjunction with an inhibitor of SOS selected from

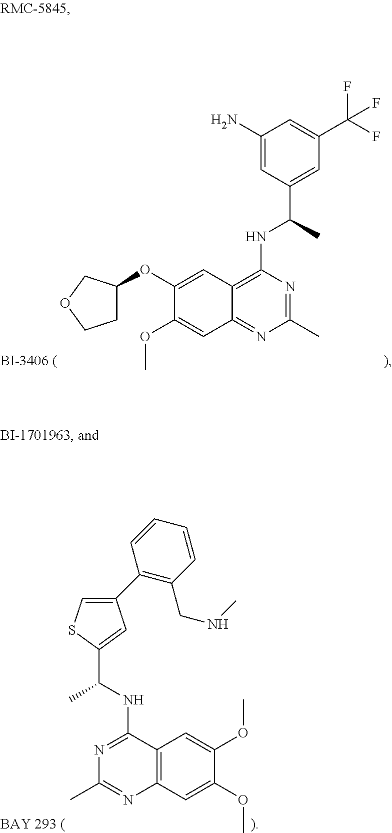 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1]

In an embodiment, the subject method comprises administering a Kras G12D inhibitor having a formula selected from formulae CA to CE, as disclosed herein, in combination with an inhibitor of EGFR selected from afatinib, erlotinib, gefitinib, lapatinib, cetuximab panitumumab, osimertinib, olnutinib, and EGF-816. In an embodiment, the subject method comprises administering a Kras G12D inhibitor having a formula selected from formulae CA to CE, as disclosed herein, in conjunction with an inhibitor of EGFR selected from afatinib, erlotinib, gefitinib, lapatinib, cetuximab panitumumab, osimertinib, olmutinib, and EGF-816. In an embodiment, the subject method comprises administering a Kras G12D inhibitor having a formula selected from formulae CA to CE, as disclosed herein, in combination with an inhibitor of MEK selected from trametinib, cobimetinib, binimetinib, selumetinib, refametinib, and AZD6244. In an embodiment, the subject method comprises administering a Kras G12D inhibitor having a formula selected from formulae CA to CE, as disclosed herein, in conjunction with an inhibitor of MEK selected from trametinib, cobimetinib, binimetinib, selumetinib, refametinib, and AZD6244. In an embodiment, the subject method comprises administering a Kras G12D inhibitor having a formula selected from formulae CA to CE, as disclosed herein, in combination with an inhibitor of ERK selected from ulixertinib, MK-8353, LTT462, AZD0364, SCH772984, BIX02189, LY3214996, and ravoxertinib. In an embodiment, the subject method comprises administering a Kras G12D inhibitor having a formula selected from formulae CA to CE, as disclosed herein, in conjunction with an inhibitor of ERK selected from ulixertinib, MK-8353, LTT462, AZD0364, SCH772984, BIX02189, LY3214996, and ravoxertinib. In an embodiment, the subject method comprises administering a Kras G12D inhibitor having a formula selected from formulae CA to CE, as disclosed herein, in combination with an inhibitor of CDK4/6 selected from palbociclib, ribociclib, and abemaciclib. In an embodiment, the subject method comprises administering a Kras G12D inhibitor having a formula selected from formulae CA to CE, as disclosed herein, in conjunction with an inhibitor of CDK4/6 selected from palbociclib, ribociclib, and abemaciclib. In an embodiment, the subject method comprises administering a Kras G12D inhibitor having a formula selected from formulae CA to CE, as disclosed herein, in combination with an inhibitor of BRAF selected from Sorafenib, Vemurafenib, Dabrafenib, Encorafenib, regorafenib, and GDC-879. In an embodiment, the subject method comprises administering a Kras G12D inhibitor having a formula selected from formulae CA to CE, as disclosed herein, in conjunction with an inhibitor of BRAF selected from Sorafenib, Vemurafenib, Dabrafenib, Encorafenib, regorafenib, and GDC-879. In an embodiment, the subject method comprises administering a Kras G12D inhibitor having the formula (CA), as disclosed herein, in combination with an inhibitor of one or more signaling molecules selected from Table 1 having a formula selected from formulae CF to CZ, DA to DZ, A to F, N to Z, Z′, AA, AA′, BB, BC, and BC′. In an embodiment, the subject method comprises administering a Kras G12D inhibitor having the formula (CA), as disclosed herein, in conjunction with an inhibitor of one or more signaling molecules selected from Table 1 having a formula selected from formulae CF to CZ, DA to DZ, A to F, N to Z, Z′, AA, AA′, BB, BC, and BC′. In an embodiment, the subject method comprises administering a Kras G12D inhibitor having the formula (CA), as disclosed herein, in combination with an inhibitor of SHP2 selected from

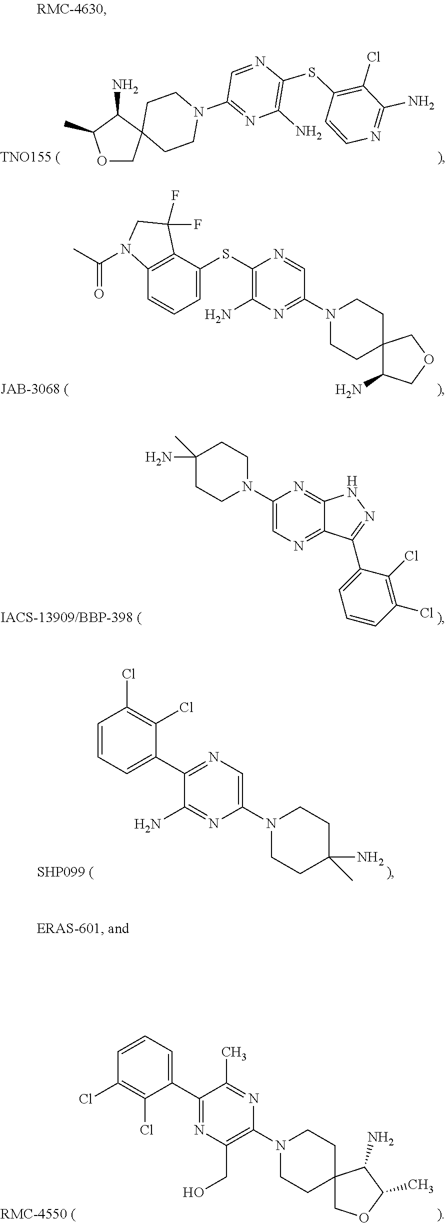 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In an embodiment, the subject method comprises administering a Kras G12D inhibitor having the formula (CA), as disclosed herein, in conjunction with an inhibitor of SHP2 selected from

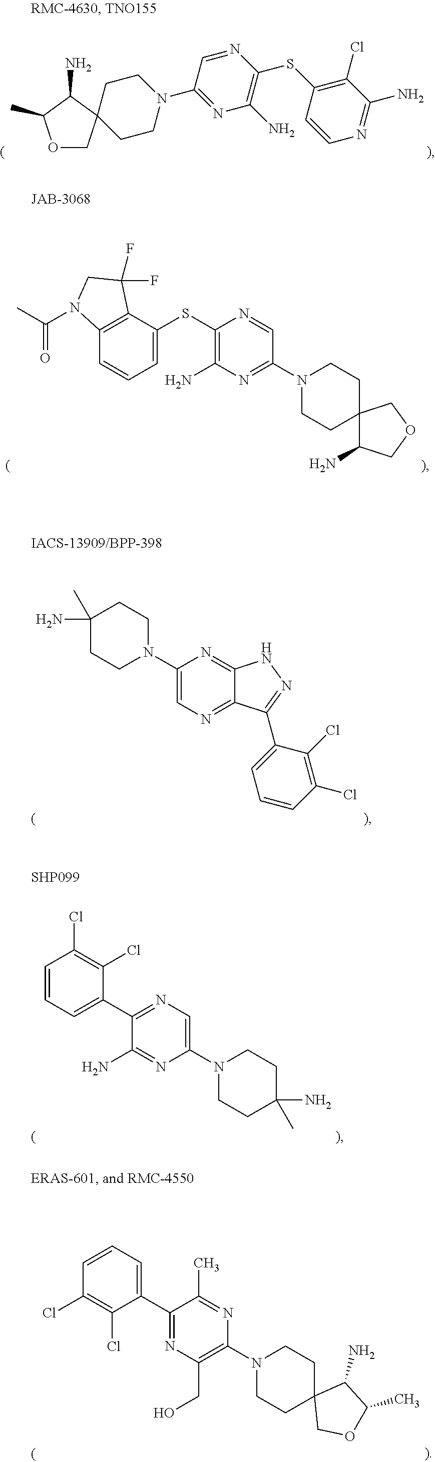

In an embodiment, the subject method comprises administering a Kras G12D inhibitor having the formula (CA) as disclosed herein, in combination with an inhibitor of SOS selected from

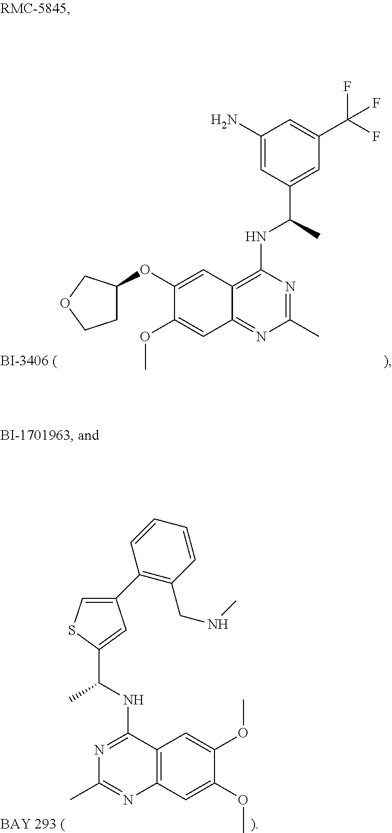 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1]

In an embodiment, the subject method comprises administering a Kras G12D inhibitor having the formula (CA), as disclosed herein, in conjunction with an inhibitor of SOS selected from

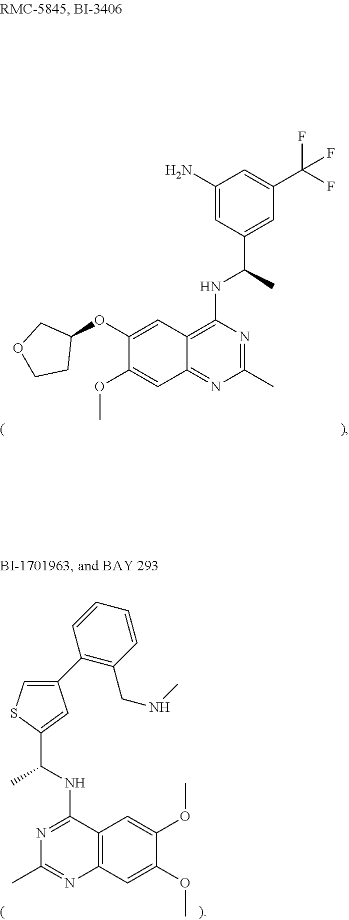

In an embodiment, the subject method comprises administering a Kras G12D inhibitor having the formula (CA), as disclosed herein, in combination with an inhibitor of EGFR selected from afatinib, erlotinib, gefitinib, lapatinib, cetuximab panitumumab, osimertinib, olmutinib, and EGF-816. In an embodiment, the subject method comprises administering a Kras G12D inhibitor having the formula (CA), as disclosed herein, in conjunction with an inhibitor of EGFR selected from afatinib, erlotinib, gefitinib, lapatinib, cetuximab panitumumab, osimertinib, olmutinib, and EGF-816. In an embodiment, the subject method comprises administering a Kras G12D inhibitor having the formula (CA), as disclosed herein, in combination with an inhibitor of MEK selected from trametinib, cobimetinib, binimetinib, selumetinib, refametinib, and AZD6244. In an embodiment, the subject method comprises administering a Kras G12D inhibitor having the formula (CA), as disclosed herein, in conjunction with an inhibitor of MEK selected from trametinib, cobimetinib, binimetinib, selumetinib, refametinib, and AZD6244. In an embodiment, the subject method comprises administering a Kras G12D inhibitor having the formula (CA), as disclosed herein, in combination with an inhibitor of ERK selected from ulixertinib, MK-8353, LTT462, AZD0364, SCH772984, BIX02189, LY3214996, and ravoxertinib. In an embodiment, the subject method comprises administering a Kras G12D inhibitor having the formula (CA), as disclosed herein, in conjunction with an inhibitor of ERK selected from ulixertinib, MK-8353, LTT462, AZD0364, SCH772984, BIX02189, LY3214996, and ravoxertinib. In an embodiment, the subject method comprises administering a Kras G12D inhibitor having the formula (CA), as disclosed herein, in combination with an inhibitor of CDK4/6 selected from palbociclib, ribociclib, and abemaciclib. In an embodiment, the subject method comprises administering a Kras G12D inhibitor having the formula (CA), as disclosed herein, in conjunction with an inhibitor of CDK4/6 selected from palbociclib, ribociclib, and abemaciclib. In an embodiment, the subject method comprises administering a Kras G12D inhibitor having the formula (CA), as disclosed herein, in combination with an inhibitor of BRAF selected from Sorafenib, Vemurafenib, Dabrafenib, Encorafenib, regorafenib, and GDC-879. In an embodiment, the subject method comprises administering a Kras G12D inhibitor having the formula (CA), as disclosed herein, in conjunction with an inhibitor of BRAF selected from Sorafenib, Vemurafenib, Dabrafenib, Encorafenib, regorafenib, and GDC-879.

In embodiments of the subject composition, the Kras G12D inhibitor has a formula selected from formulae CA to CE, as disclosed herein, and the inhibitor of one or more signaling molecules selected from Table 1 has a formula selected from formulae CF to CZ, DA to DZ, A to F, N to Z, Z′, AA, AA′, BB, BC, and BC′. In embodiments of the subject composition, the Kras G12D inhibitor has a formula selected from formulae CA to CE, as disclosed herein, and the inhibitor of one or more signaling molecules selected from Table 1 is an inhibitor of SHP2 selected from

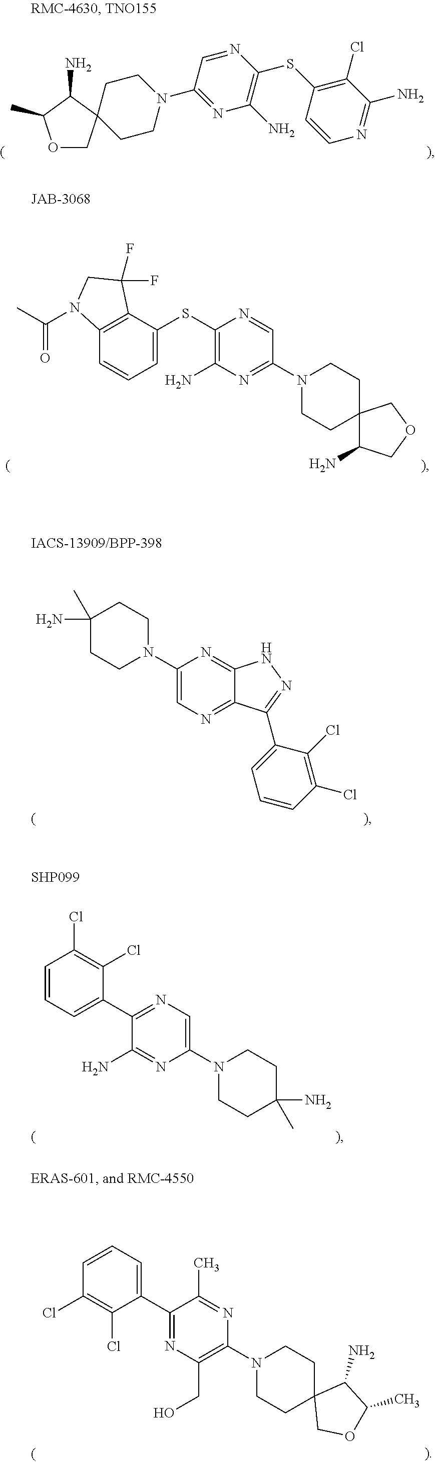

In embodiments of the subject composition, the Kras G12D inhibitor has a formula selected from formulae CA to CE, as disclosed herein, and the inhibitor of one or more signaling molecules selected from Table 1 is an inhibitor of SOS selected from

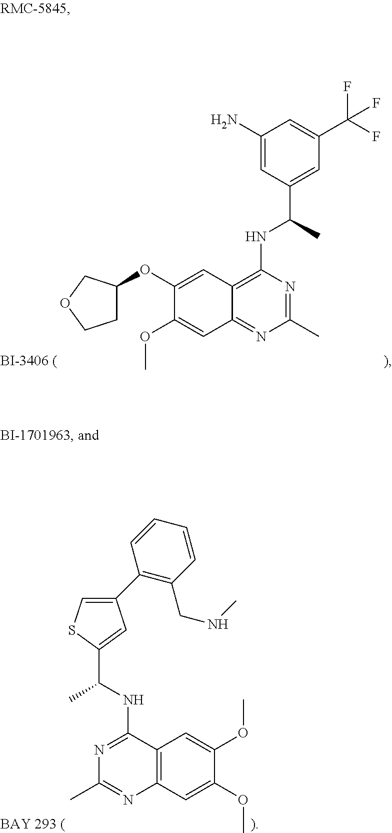 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1]

In embodiments of the subject composition, the Kras G12D inhibitor has a formula selected from formulae CA to CE, as disclosed herein, and the inhibitor of one or more signaling molecules selected from Table 1 is an inhibitor of EGFR selected from afatinib, erlotinib, gefitinib, lapatinib, cetuximab panitumumab, osimertinib, olmutinib, and EGF-816. In embodiments of the subject composition, the Kras G12D inhibitor has a formula selected from formulae CA to CE, as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has a formula selected from formulae CA to CE, as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has a formula selected from formulae CA to CE, as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has a formula selected from formulae CA to CE, as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879. In embodiments of the subject composition, the Kras G12D inhibitor has the formula (CA), as disclosed herein, and the inhibitor of one or more signaling molecules selected from Table 1 has a formula selected from formulae CF to CZ, DA to DZ, A to F, N to Z, Z′, AA, AA′, BB, BC, and BC′. In embodiments of the subject composition, the Kras G12D inhibitor has the formula (CA), as disclosed herein, and the inhibitor of one or more signaling molecules selected from Table 1 is an inhibitor of SHP2 selected from

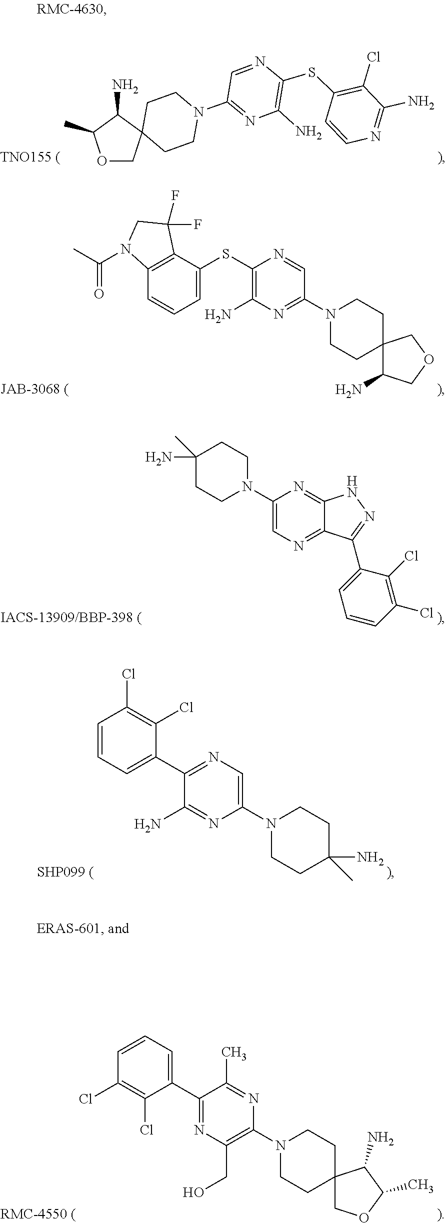 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CA), and the inhibitor of one or more signaling molecules selected from Table 1 is an inhibitor of SOS selected from

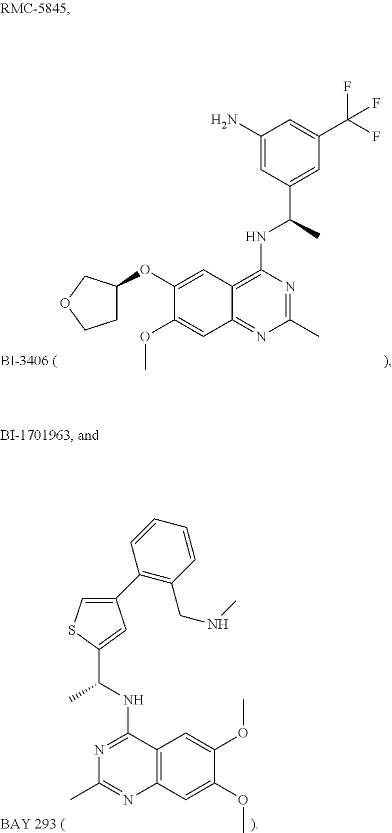 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1]

In embodiments of the subject composition, the Kras G12D inhibitor has the formula (CA), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olmutinib, and EGF-816. In embodiments of the subject composition, the Kras G12D inhibitor has the formula (CA), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CA), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CA), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CA), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows a degree of growth inhibition of ASPC1 pancreatic cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations), or (iii) in combination with each other respectively;

FIG. 1B shows a degree of synergy across the drug combinations in FIG. 1A, as calculated by using the BLISS independent model;

FIG. 2A shows a degree of growth inhibition of Panc04.03 pancreatic cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations) alone, or (iii) in combination with each other respectively;

FIG. 2B shows a degree of synergy across the combinations in FIG. 2A, as calculated by using the BLISS independent model;

FIG. 3A shows a degree of growth inhibition of A427 non-small cell lung cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations) alone, or (iii) in combination with each other respectively;

FIG. 3B shows a degree of synergy across the combinations in FIG. 3A, as calculated by using the BLISS independent model;

FIG. 4A shows a degree of growth inhibition of Ls174T colorectal cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations) alone, or (iii) in combination with each other respectively;

FIG. 4B shows a degree of synergy across the combinations in FIG. 4A, as calculated by using the BLISS independent model;

FIG. 5A shows a degree of growth inhibition of Ls513 colorectal cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations) alone, or (iii) in combination with each other respectively;

FIG. 5B shows a degree of synergy across the combinations in FIG. 5A, as calculated by using the BLISS independent model;

FIG. 6A shows a degree of growth inhibition of AGS gastric cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations) alone, or (iii) in combination with each other respectively;

FIG. 6B shows a degree of synergy across the combinations in FIG. 6A, as calculated by using the BLISS independent model;

FIG. 7A shows a degree of growth inhibition of HEClA endometrial cancer cells upon treatment with (i) a KrasG12D inhibitor alone (Compound A, Compound B, or Compound C, at various concentrations), (ii) an inhibitor of another signaling molecule (SOS, SHP2, EGFR, MEK, CDK4/6, or PI3Ka, at various concentrations) alone, or (iii) in combination with each other respectively;

FIG. 7B shows a degree of synergy across the combinations in FIG. 7A, as calculated by using the BLISS independent model;

FIG. 8 shows ranking of the BLISS synergy scores of various inhibitor combinations across 7 cell lines in accordance with FIGS. 1B, 2B, 3B, 4B, 5B, 6B, and 7B;

FIG. 9 shows degrees of growth inhibition of cancer cells (ASPC1 cells, Ls513 cells, or A427 cells) upon treatment with an inhibitor of another signaling molecule (SOS, SHP2, EGFR, MEK, CDK4/6, or PI3Ka) alone, in combination with (1) Kras knockdown by lentiviral-delivered shRNAs or (2) a control shRNAs (see line graphs). FIG. 9 also shows degrees of synergy across the combinations, as calculated by using the BLISS independent model (see pie charts);

FIG. 10 shows an exemplary Ras signaling pathway;

FIG. 11 shows the amino acid sequence of human K-Ras isoform 4b (K-Ras4b or K-Ras isoform 2) (SEQ ID NO. 1);

FIG. 12 shows the growth inhibition (IC₅₀ in nM) observed across 8 cell lines upon treatment with (i) a KrasG12D inhibitor alone (Compound D, Compound E, Compound F, or Compound G), (ii) the KrasG12D inhibitor in combination with a SOS inhibitor (1 μM), or (iii) the KrasG12D inhibitor in combination with a SOS inhibitor (5 μM).

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

Unless defined otherwise, all technical and scientific terms used herein have the same meaning as is commonly understood by one of skill in the art to which this invention belongs.

The term “a” and “an” refers to one or to more than one (i.e., to at least one) of the grammatical object of the article. By way of example, “an element” means one element or more than one element.

“About” as used herein when referring to a measurable value such as an amount, a duration, and the like, is meant to encompass variations of ±10% of a stated number or value.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fuhmaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), Morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

The term “small molecule” refers to one or more members from the group comprising an ion, a lipid, a chemical compound (e.g., natural or synthetic), and an amino acid (e.g., natural or synthetic), and a polypeptide (e.g., peptide or protein).

The term “nucleic acid agent” refers to an inhibitory agent capable of downregulating (e.g., reducing or inhibiting) expression and/or activity of a target moiety (e.g., a protein or a gene encoding thereof). In some embodiments, a nucleic acid agent may consist of a nucleic acid molecule. In some embodiments, a nucleic acid agent may comprise a nucleic acid molecule. In some embodiments, a nucleic acid agent may comprise a nucleic acid molecule and a non-nucleic acid molecule. The nucleic acid molecule and the non-nucleic acid molecule may be operatively coupled to each other to yield the inhibitory effect on the target moiety. The nucleic acid molecule and the non-nucleic acid molecule may be coupled (e.g., covalently and/or non-covalently) to each other. In some cases, the nucleic acid molecule and the non-nucleic acid molecule can be linked to each other via a linker. In some cases, the nucleic acid molecule can be configured to bind to the non-nucleic acid molecule. In some cases, the non-nucleic acid molecule can be configured to bind to the nucleic acid molecule. Non-limiting examples of the non-nucleic acid molecule include a small molecule, a polypeptide (e.g., an enzyme), etc. In some cases, the non-nucleic acid molecule is a nuclease, e.g., an endonuclease.

The term “endonuclease” refers to an enzyme capable of regulating expression or activity of a gene and/or edit a nucleic acid sequence, whether exogenous or endogenous. An endonuclease can regulate expression of a gene at the transcription level and/or the translation level. An endonuclease can regulate gene expression at the transcription level, for example, by regulating the production of mRNA from DNA, such as chromosomal DNA or cDNA. In some embodiments, an endonuclease recruits at least one transcription factor that binds to a specific DNA sequence, thereby controlling the rate of transcription of genetic information from DNA to mRNA. An endonuclease can itself bind to DNA and regulate transcription by physical obstruction, for example preventing proteins such as RNA polymerase and other associated proteins from assembling on a DNA template. An endonuclease can regulate expression of a gene at the translation level, for example, by regulating the production of protein from mRNA template. In some embodiments, an actuator moiety regulates gene expression by affecting the stability of an mRNA transcript. In some embodiments, an endonuclease regulates expression of a gene by editing a nucleic acid sequence (e.g., a region of a genome). In some embodiments, an endonuclease regulates expression of a gene by editing an mRNA template. Editing a nucleic acid sequence can, in some cases, alter the underlying template for gene expression. Non-limiting examples of an endonuclease include one or more members from the group comprising CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)).

A Cas protein herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive (i.e., deactivated Cas, dead Cas, dGas), partially active, constitutively active, fully active, inducible active and/or more active, (e.g. more than the wild type homologue of the protein or polypeptide.). Non-limiting examples of a Cas protein can include Cas9, Cas12a (i.e., Cpf1), Cas12b (i.e., C2c1, Cpf2), Cas12c (i.e., G2c3), Cas12d (i.e., CasY), Cas12e (i.e., CasX). Cas13a (i.e., C2c2), Cas13b (i.e., C2c6), Cas13c (i.e., G2c7), Cas13d (i.e., Casrx), and functional variants thereof. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA. A Cas protein can be operatively coupled to a guide nucleic acid (e.g., a guide RNA (gRNA)). In some cases, a Cas protein can be complexed with a guide nucleic acid for targeted regulation of gene expression and/or activity or nucleic acid editing. A nucleic acid-guided Cas protein can specifically bind a target polynucleotide (e.g., DNA or RNA) in a sequence-dependent manner. In some embodiments, a Cas protein may be a part of a fusion comprising (i) the Cas protein and (ii) at least one additional moiety (e.g., at least one additional polypeptide sequence).

As used herein, “fusion” can refer to a protein comprising one or more non-native sequences (e.g., moieties). A fusion can comprise one or more of the same non-native sequences. A fusion can comprise one or more of different non-native sequences. A fusion can be a chimera. A fusion can comprise a tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like).

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion comprising a Cas protein. A fusion protein can be a non-native sequence in a Cas protein.

The term “guide nucleic acid” refers to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A guide nucleic acid can comprise two polynucleotide chains and can be called a “double guide nucleic acid.” If not otherwise specified, the term “guide nucleic acid” can be inclusive, referring to both single guide nucleic acids and double guide nucleic acids. A guide nucleic acid can comprise a segment that can be referred to as a “nucleic acid-targeting segment” or a “nucleic acid-targeting sequence.” A nucleic acid-targeting nucleic acid can comprise a segment that can be referred to as a “protein binding segment” or “protein binding sequence” or “Cas protein binding segment.”

The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

The term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject).

The term “administer,” “administering,” “administration,” and derivatives thereof refer to the methods that may be used to enable delivery of a composition to the desired site of biological action. These methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Administration is by any route, including parenteral. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

The term “sub-therapeutic amount” or “sub-effective amount” refers to the amount of an agent that is less than the effective amount for that agent, but when combined with an effective or sub-therapeutic amount of a different agent can produce a desired result, due to, for example, synergy in the resulting efficacious effects, and/or reduced side effects by the combination of (i) the sub-therapeutic amount of the agent and (ii) the different agent (e.g., one or more different agents). For example, an agent can be approved for clinical use at a defined dose or range thereof (e.g., 150 milligrams per day (mg/d)) over the course of one or more administrations, and a sub-therapeutic amount of such agent can be lower than the approved dose or range thereof by at least about 0.1-fold, 0.2-fold, 0.5-fold, 1-fold, 2-fold, 5-fold, 10-fold, 20-fold, 50-fold, 100-fold, 200-fold, 500-fold, 1,000-fold, 2,000-fold, 5,000-fold, 10,000-fold, 20,000-fold, 50,000-fold, 100,000-fold, or more. The sub-therapeutic amount of such agent can be lower than the approved dose or range thereof by at most about 100,000-fold, 50,000-fold, 20,000-fold, 10,000-fold, 5,000-fold, 2,000-fold, 1,000-fold, 500-fold, 200-fold, 100-fold, 50-fold, 20-fold, 10-fold, 5-fold, 2-fold, 1-fold, 0.5-fold, 0.2-fold, 0.1-fold or less. A sub-therapeutic amount of an agent can be achieved by reducing the amount of the agent per dosage and/or by reducing the number of administrations (or cycles) of the agent to the subject.

The term “synergistic” or “synergizing” effect refers to when a desired effect (e.g., one or more different effects) of a combination (or combination treatment) comprising two or more different therapeutic components (e.g., two or more different therapies, two or more therapeutic agents, etc.) is greater than (i) the effect of each therapeutic component alone and/or (ii) the sum of the effect of each therapeutic component alone when administered individually (e.g., the sum of individual effects). The synergistic effect can be at least about 1%, 2%, 3%, 4%, 5%, 6%, 7%, 8%, 9%, 10%, 15%, 20%, 30%, 40%, 50%, 60%, 70%, 80%, 90%, 100%, 110%, 120%, 150%, 200%, 300%, 400%, 500%, 600%, 700%, 800%, 900%, 1,000%, 5,000%, or more than (i) the effect of each therapeutic component alone and/or (ii) the sum of individual effects. The synergistic effect can be at most about 5,000%, 1,000%, 900%, 800%, 700%, 600%, 500%, 400%, 300%, 200%, 150%, 120%, 110%, 100%, 90%, 80%, 70%, 60%, 50%, 40%, 30%, 20%, 15%, 10%, 9%, 8%, 7%, 6%, 5%, 4%, 3%, 2%, 1%, or less that (i) the effect of each therapeutic component alone and/or (ii) the sum of individual effects. The effect can be any of the measurable effects described herein. To yield the synergistic effect, the two or more different therapeutic components of the combination treatment as disclosed herein can be adminstered concurrently or sequentially. For example, a synergistic effect of a combination comprising a first agent at a sub-therapeutic amount and a second agent at a sub-therapeutic amount can yield a desired therapeutic outcome (e.g., in treating cancer) that is comparable (e.g., substantially the same) or better than (i) the therapeutic outcome of each therapeutic component alone at the therapeutically effective amount and/or (ii) the sum of individual effects.

The term “IC50” refers to the half maximal inhibitory amount (e.g., concentration) of an inhibitor in inhibiting a biological or biochemical effect. IC50 can be a quantitative measure that indicates how much of a particular inhibitor is needed to inhibit a given biological or biochemical effect (e.g., expression and/or activity level of a gene/protein of interest, growth, or growth rate of a cell, etc.) by substantially half (e.g., about 50%). For example, determination of IC50 can be made by determining and constructing a dose-response curve and examining the effect of different concentrations of an inhibitor on reducing cell growth (e.g., inhibiting proliferation of cancer cells), and determining the concentration of the inhibitor at which 50% inhibition of cell growth is observed.

The term “combination”, as applied to agents including inhibitors disclosed herein, refers to the use of two or more agents (e.g., a Kras G12D inhibitor and at least another inhibitor against a different signaling molecule selected from Table 1) in vitro, in vivo, or ex-vivo. This two or more agent combination can be formulated in one single formulation, or in separate formulation(s). A combination treatment or therapy with two or more agents can be carried out in any temporal order, administered simultaneously or separately.

The term “conjunction” refers to a temporal aspect of the use of two or more agents (e.g., a Kras G12D inhibitor and at least another inhibitor against a different signaling molecule selected from Table 1) in vitro, in vivo, or ex-vivo. For example, one agent of a set of agents of interest can be administered prior to, subsequent to, or concurrently with the administration of a second agent of the set. The two or more agents used in conjunction or conjunctively can be formulated in a single formulation or in separate formulation(s).

An “antigen” is a moiety or molecule that contains an epitope, and, as such, also specifically binds to an antibody.

An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or V_{H}H), a heavy chain found in sharks (V-NAR domain), a single domain antibody (sdAb, i.e., “nanobody”) that comprises a single antigen-binding domain, Fv, Fd, Fab, Fab′, F(ab′)2, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

The term “diseased cell” refers to the state of a cell, tissue, or organism that diverges from the normal or healthy state. A diseased cell may result from the influence of a pathogen, a toxic substance, irradiation, or cell internal deregulation (e.g., genetic mutation). In an example, a diseased cell is a cell that has been infected with a pathogenic virus. In an example, a diseased cell is a malignant cell or neoplastic cell that may constitute or give rise to cancer in a subject (e.g., a mammal such as a human subject).

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (or K-Ras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof.

The terms “X-ras,” “X-Ras,” “Xras,”, “XRas”, and “XRAS” can be used interchangeable herein, wherein X is selected from the group consisting of K, H, N, M, E, and R. For example, the terms “K-ras,” “K-Ras,” “Kras,”, “KRas”, and “KRAS” are used interchangeable herein.

The terms “Mutant Ras” and “Ras mutant,” as used interchangeably herein, refer to a Ras protein with one or more amino acid mutations, such as with respect to a common reference sequence such as a wild-type (WT) sequence. In some embodiments, a mutant Ras is selected from a mutant KRAS, mutant HRAS, mutant NRAS, mutant MRAS, mutant ERAS, mutant RRAS2, mutant RALA, mutant RALB, mutant RIT1, and any combination thereof, such as from a mutant KRAS, mutant HRAS, mutant NRAS, mutant RALA, mutant RALB, and any combination thereof. In some embodiments, a mutation can be an introduced mutation, a naturally occurring mutation, or a non-naturally occurring mutation. In some embodiments, a mutation can be a substitution (e.g., a substituted amino acid), insertion (e.g., addition of one or more amino acids), or deletion (e.g., removal of one or more amino acids).

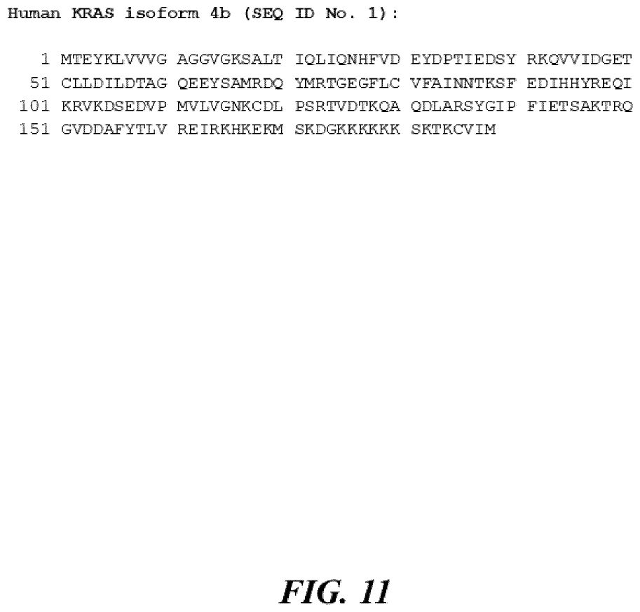

In some embodiments, two or more mutations can be consecutive, non-consecutive, or a combination thereof. In some embodiments, a mutation can be present at any position of Ras. In some embodiments, a mutation can be present at position 12, 13, 62, 92, 95, or any combination thereof of Ras relative to SEQ ID No. 1 (FIG. 11) when optimally aligned. For example, a mutation can be present at position 12 (e.g., G12D) of KRas relative to SEQ ID NO. 1. In some embodiments, a mutant Ras may comprise about or at least about 1, 2, 3, 4, 5, 6, 7, 8, 9, 10, 11, 12, 13, 14, 15, 16, 17, 18, 19, 20, 25, 30, 35, 40, 45, 50, or more than 50 mutations. In some embodiments, a mutant Ras may comprise up to about 1, 2, 3, 4, 5, 6, 7, 8, 9, 10, 11, 12, 13, 14, 15, 16, 17, 18, 19, 20, 25, 30, 35, 40, 45, or 50 mutations. In some embodiments, the mutant Ras is about or up to about 500, 400, 300, 250, 240, 233, 230, 220, 219, 210, 208, 206, 204, 200, 195, 190, 189, 188, 187, 186, 185, 180, 175, 174, 173, 172, 171, 170, 169, 168, 167, 166, 165, 160, 155, 150, 125, 100, 90, 80, 70, 60, 50, or fewer than 50 amino acids in length. In some embodiments, an amino acid of a mutation is a proteinogenic, natural, standard, non-standard, non-canonical, essential, non-essential, or non-natural amino acid. In some embodiments, an amino acid of a mutation has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, a mutation comprises a reactive moiety. In some embodiments, a substituted amino acid comprises a reactive moiety. In some embodiments, a mutant Ras can be further modified, such as by conjugation with a detectable label. In some embodiments, a mutant Ras is a full-length or truncated polypeptide. For example, a mutant Ras can be a truncated polypeptide comprising residues 1-169 or residues 11-183 (e.g., residues 11-183 of a mutant RALA or mutant RALB).

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

Except where otherwise defined herein, an “alkyl” group refers to a straight or branched hydrocarbon chain radical consisting solely of carbon and hydrogen atoms, containing no unsaturation. In some embodiments, the “alkyl” group may have 1 to 18, 1 to 12, 1 to 10, 1 to 8, or 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆alkyl” or similar designations. By way of example only, “C₁-C₆alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, and hexyl. Alkyl groups can be substituted or unsubstituted. Depending on the structure, an alkyl group can be a monoradical or a diradical (i.e., an alkylene group).

Except where otherwise defined herein, an “alkoxy” refers to a “—O-alkyl” group, where alkyl is as defined herein.

Except where otherwise defined herein, the term “alkenyl” refers to a straight or branched hydrocarbon chain radical group consisting solely of carbon and hydrogen atoms, containing at least one carbon-carbon double bond. Non-limiting examples of an alkenyl group include —CH═CH₂, —C(CH₃)═CH₂, —CH═CHCH₃, —CH═C(CH₃)₂ and —C(CH₃)═CHCH₃. In some embodiments, an alkenyl groups may have 2 to 6 carbons. Alkenyl groups can be substituted or unsubstituted. Depending on the structure, an alkenyl group can be a monoradical or a diradical (i.e., an alkenylene group).

Except where otherwise defined herein, the term “alkynyl” refers to a straight or branched hydrocarbon chain radical group consisting solely of carbon and hydrogen atoms, containing at least one carbon-carbon triple bond. Non-limiting examples of an alkynyl group include —C═CH, —C═CCH₃, —C═CCH₂CH₃ and —C═CCH₂CH₂CH₃. In some embodiments, an alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

Except where otherwise defined herein, the term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂ group, where alkyl is as defined herein.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

Except where otherwise defined herein, the term “aryl” refers to a monocyclic aromatic ring wherein each of the atoms forming the ring is a carbon atom (e.g., phenyl) or a polycyclic ring system (e.g., bicyclic or tricyclic) wherein 1) at least one ring is carbocyclic and aromatic, 2) all aromatic rings in the rings system are carbocyclic, and 3) a bond to the remainder of the compound is directly bonded to a carbocyclic aromatic ring of the aryl ring system. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group). As used herein, the aryl radical is a monocyclic, bicyclic, or tricyclic ring system. In some embodiments the aryl is a “fused ring aryl” wherein the aryl ring is fused with a cycloalkyl or a heterocycloalkyl ring.

Except where otherwise defined herein, “Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

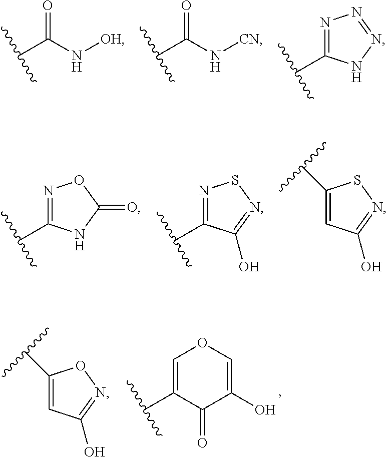

and the like.

Except where otherwise defined herein, the term “cycloalkyl” refers to a monocyclic carbocyclic saturated or partially unsaturated non-aromatic ring or a polycyclic carbocyclic (i.e., does not include heteroatom(s)) ring system (e.g., bicyclic or tricyclic) wherein 1) at least one ring is carbocyclic saturated or partially unsaturated and non-aromatic and 2) a bond to the remainder of the compound is directly bonded to a carbocyclic saturated or partially unsaturated non-aromatic ring of the ring system. Cycloalkyls may be saturated or partially unsaturated. In some embodiments, a cycloalkyl ring is a spirocyclic cycloalkyl ring. In some embodiments, cycloalkyl groups include groups having from 3 to 10 ring atoms. Depending on the structure, a cycloalkyl group can be a monoradical or a diradical (i.e., a cycloalkylene group).

Except where otherwise defined herein, the terms “heteroaryl” or, alternatively, “heteroaromatic” refers to an monocyclic aryl group that includes one or more ring heteroatoms selected from nitrogen, oxygen and sulfur; or a polycyclic ring system (e.g., bicyclic or tricyclic) wherein 1) at least one ring is aromatic and includes one or more heteroatoms selected from nitrogen, oxygen and sulfur and 2) a bond to the remainder of the compound is directly bonded to an aromatic ring including one or more heteroatoms selected from nitrogen, oxygen and sulfur or an aromatic ring directly bonded (e.g., fused) to an aromatic ring including one or more heteroatoms selected from nitrogen, oxygen and sulfur, of the aryl ring system. As used herein, the heteroaryl radical is a monocyclic, bicyclic, or tricyclic ring system, wherein at least one of the rings in the ring system is fully unsaturated (i.e., aromatic). In some embodiments is a “fused ring heteroaryl” wherein the heteroaryl ring is fused with a cycloalkyl, aryl, or heterocycloalkyl ring. An N-containing “heteroaromatic” or “heteroaryl” moiety refers to an aromatic group in which at least one of the skeletal atoms of the ring is a nitrogen atom. Depending on the structure, a heteroaryl group can be a monoradical or a diradical (i.e., a heteroarylene group).

Except where otherwise defined herein, a “heterocycloalkyl” group or “heteroalicyclic” group or “heterocyclyl” refers to a cycloalkyl group, wherein at least one skeletal ring atom of a saturated or partially unsaturated non-aromatic ring is a heteroatom selected from nitrogen, oxygen and sulfur. A heterocycloalkyl refers to a monocyclic saturated or partially unsaturated non-aromatic ring including one or more heteroatoms or a polycyclic ring system (e.g., bicyclic or tricyclic) wherein 1) at least one ring is saturated or partially unsaturated, non-aromatic, and includes one or more heteroatoms and 2) a bond to the remainder of the compound is directly bonded to a ring of the ring system that is a saturated or partially unsaturated and non-aromatic ring that includes one or more heteroatoms or a non-aromatic ring directly bonded (e.g., fused) to a saturated or partially unsaturated and non-aromatic ring that includes one or more heteroatoms of the ring system. Heterocycloalkyls may be saturated or partially unsaturated. The term heterocycloalkyl also includes all ring forms of the carbohydrates, including but not limited to the monosaccharides, the disaccharides and the oligosaccharides. In some embodiments, a heterocycloalkyl ring is a spirocyclic heterocycloalkyl ring. In some embodiments, a heterocycloalkyl ring is a bridged heterocycloalkyl ring. Unless otherwise noted, heterocycloalkyls have from 2 to 13 carbons in the ring or ring system. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Depending on the structure, a heterocycloalkyl group can be a monoradical or a diradical (i.e., a heterocycloalkylene group).

The term “halo” or, alternatively, “halogen” means fluoro, chloro, bromo and iodo.

The abbreviations “Fmoc”, “Ac”, “Bn”, “PMB”, “Tr”, “Ts”, “Boc”, and “Cbz” are used in accordance with their well understood common meanings in Chemistry and mean the monovalent chemical substituents fluorenylmethyloxycarbonyl, acetyl, benzyl, p-methoxybenzyl, trityl or triphenylmethyl, tosyl, tert-butyloxycarbonyl, and carbobenzyloxy, respectively.

Except where otherwise defined herein, the term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, and the like.

Except where otherwise defined herein, the terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

Except where otherwise defined herein, the term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “oxo” refers to the ═O radical.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

The suffix “-di-yl” will be understood to mean the substituent or linker is a divalent substituent or linker.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

“Optional” or “optionally” means that a subsequently described event or circumstance may or may not occur and that the description includes instances when the event or circumstance occurs and instances in which it does not.

Except where otherwise defined herein, the term “optionally substituted” or “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L⁵R⁵, wherein each L⁵ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R^{s} is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Methods

The methods disclosed herein have a wide range of applications in therapeutics, diagnostics, and other biomedical research. The methods disclosed herein can utilize at least one therapeutic agent (e.g., a single therapeutic agent or a plurality of different therapeutic agents). Certain aspects of the present disclosure provides a method of using a combination treatment to treat a cell (e.g, a cancer cell) or a subject in need thereof (e.g., a cancer patient), and the combination treatment can comprise a plurality of different therapeutic agents (e.g., at least 2, 3, 4, 5, 6, 7, 8, 9, 10, or more different drugs).

A combination treatment comprising a plurality of different therapeutic agents, as disclosed herein, can achieve one or more desired therapeutic actions or outcomes, including, but not limited to, (i) reduced expression and/or activity levels of disease related markers (e.g., cancer related genes, such as Kras G12D, etc.), (ii) reduced angiogenesis (e.g., at a tumor site), (iii) reduced cancer cell proliferation, and/or (iv) reduced tumor growth or progression, substantial removal of tumor, and/or delayed or prevention of metastasis. The combination treatment can achieve such desired actions or outcomes, while providing one or more superior advantages including, but not limited to, (1) therapeutic efficacy with a synergistic effect, (2) decreased amount (e.g., dosage regimen, number dosages, etc.) of one, more, or all of the therapeutic agents of the plurality of different therapeutic agents, and/or (3) avoiding, limiting, or reducing any undesirable side-effects associated with the use of any one of the plurality of different therapeutic agents when used in the therapeutically effective amount or clinically approved amount. Non-limiting examples of undesirable side-effects associated with anti-cancer agents can include nausea, myelosuppression, alopecia, vomiting, stomatitis, and also cardio-toxicity.

In some aspects, the combination treatment as disclosed herein can utilize an agent that can be therapeutically sub-optimal when used alone, to yield an overall therapeutic efficacy (e.g., promoting one or more desired therapeutic outcomes and/or reducing undesirable side-effects). For example, the therapeutically sub-optimal agent (e.g., a drug, such as a Kras G12D inhibitor) can exhibit one or more sub-optimal pharmacokinetics (PK) parameters, such as (i) poor membrane permeability, (ii) low bioavailability, (iii) short half-life, (iv) rapid metabolism, (v) rapid clearance, (vi) low area under the curve (AUC), (vii) low therapeutic index, and/or (viii) small therapeutic window (or short duration of action). However, when used with one or more additional agents, the resulting combination treatment can exhibit a desired therapeutic efficacy (e.g., by exhibiting the synergistic effect), despite the one or more sub-optimal PK parameters. Thus, the combination treatment, as disclosed herein, can expand the library of agents (e.g., drugs) that can be used to treat a disease or a condition of a subject, e.g., thereby providing more clinical treatment options to patients.

In some cases, a Kras G12D inhibitor may exhibit an undesirable or poor PK parameter when used alone, but when used along with at least one additional inhibitor (e.g., that of another signaling molecule, such as SOS, SHP2, EGFR, PI3K, etc.), the resulting combination treatment can be applicable for a therapeutic application (e.g., treatment of cancer). In an example, a Kras G12D inhibitor may exhibit a poor membrane permeability characterized by (i) a logarithmic effective permeability of less than about 0 (e.g., less than to −2, −3, −4, or −5, wherein −5 is less than −2) as ascertained by a potential of mean force (PMF) molecular dynamics (MD) simulation and/or (ii) a logarithmic effective permeability of less than about −6 (e.g., −7 is less than −6) as ascertained by an in vitro Parallel Artificial Membrane Permeability Assay (PAMPA), but when used in a combination treatment as disclosed herein, the combination treatment can be effective in inducing an outcome that is therapeutically relevant (e.g., cancer cell growth inhibition, tumor reduction, etc.). In another example, a Kras G12D inhibitor may exhibit a low bioavailability of less than about 50% F, 40% F, or 30% F oral bioavailability, but when used in a combination treatment as disclosed herein, the combination treatment can be effective in inducing an outcome that is therapeutically relevant. In another example, a Kras G12D inhibitor may exhibit a low elimination half-life of less than about 12 hours, 11 hours, 10 hours, 9 hours, or 8 hours oral bioavailability (e.g., less than about 6 hours), but when used in a combination treatment as disclosed herein, the combination treatment can be effective in inducing an outcome that is therapeutically relevant. The therapeutic outcome of the combination treatment as disclosed herein can be, e.g., inhibition of cancer cell proliferation by at least about 20%, 40%, 50%, 60%, 70%, 80%, 90%, or more, as compared to that by the Kras G12D inhibitor alone.

In an aspect, the present disclosure provides a method comprising regulating (e.g., upregulating, downregulating) cell signaling in a cell. Non-limiting examples of the cell signaling include cell growth, differentiation, de-differentiation, survival, proliferation, migration, metabolism, cytotoxicity (e.g., against a target cell). In some cases, cell signaling can be regulated by downregulating, in the cell, expression or activity of at least one signaling molecule of the cell. The at least one signaling molecule of the cell can comprise at least about 1, 2, 3, 4, 5, 6, 7, 8, 9, 10, 11, 12, 13, 14, 15, 16, 17, 18, 19, 20, or more signaling molecules (e.g., signaling proteins and/or adaptors thereof). The at least one signaling molecule of the cell can comprise at most about 20, 19, 18, 17, 16, 15, 14, 13, 12, 11, 10, 9, 8, 7, 6, 5, 4, 3, 2, or 1 signaling molecule, including but not limited to those described in Table 1. In some embodiments, the at least one signaling molecule comprises a plurality of signaling molecules that are parts of (i) the same signaling pathway of the cell or (ii) different signaling pathways of the cell. In some embodiments, the present disclosure provides a method comprising: administering (a) an inhibitor against a Ras G12D (e.g., KRas G12D) protein; and (b) an inhibitor against a Ras G12C (e.g., KRas G12C) protein. In some other embodiments, the present disclosure provides a method comprising: administering an inhibitor against (a) a Ras protein (e.g., a mutated Ras protein, such as Kras G12D), an inhibitor against (b) one or more signaling molecules selected from Table 1, and an inhibitor against (c) a Ras G12C (e.g., KRas G12C) protein.

Non-limiting examples of a signaling pathway include AKT signaling pathway, angiopoietin-TIE2 signaling pathway, major histocompatibility complex (MHC) signaling pathway, death receptor pathway (e.g., for apoptosis), APRIL pathway, B-cell development pathway, B-cell receptor (BCR) pathway, bone morphogenetic protein (BMP) pathway, G protein-coupled receptor (GPCR) superfamily (e.g., C—C motif chemokine receptor type 5 (CCR5) signaling pathway), T-cell receptor (TCR) signaling pathway, CTLA4 signaling pathway, receptor tyrosine kinase (RTK) pathway (e.g., ErbB family pathway), Fas signaling pathway, fibroblast growth factor (FGF) pathway, Granzyme A (GzmA) pathway, GSK3 signaling pathway, cytokine (e.g., IL-2, IL-6, IL-10, IL-22, interferon, etc.) signaling pathway, JAK/STAT pathway, mitogen-activated protein kinases (MAPK) family pathway, p53-mediated apoptosis pathway, phosphoinositide 3-kinase (PI3K) pathway, receptor activator of nuclear factor kappa-B ligand (RANK) pathway, transforming growth factor (TGF) receptor pathway, tumor necrosis factor (TNF) superfamily signaling pathway, vascular endothelial growth factor (VEGF) receptor pathway, epidermal growth factor receptor (EGFR) pathway, etc.

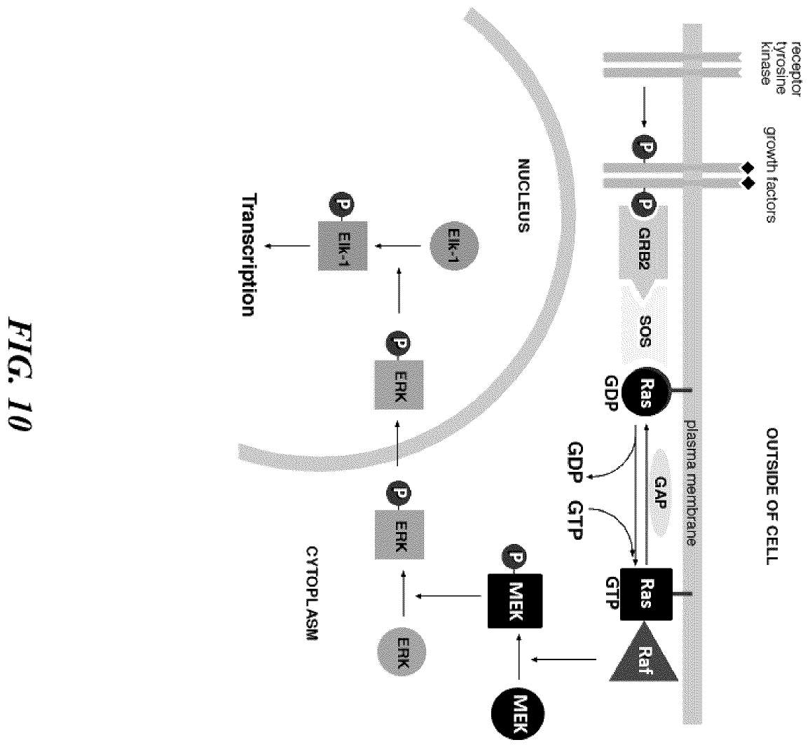

In some embodiments, the at least one signaling molecule are involved in regulating cell proliferation signaling in the cell. The at least one signaling molecule can comprise Ras or Ras mutant. For example, the at least one signaling molecule can comprise Kras or Kras mutant, such as Kras G12D. Alternatively or in addition to, the at least one signaling molecule can comprise one or more signaling molecules selected from Table 1. In some examples, the at least one signaling molecule can comprise (i) Kras G12D and (ii) one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28 or 29) signaling molecules from Table 1. The at least one signaling molecule can comprise (i) Kras G12D and (ii) at most about 5, 4, 3, 2, or signaling molecule from Table 1. In an example, the at least one signaling molecule comprises (1) Kras G12D and (2) one or more members selected from the group consisting of (i) SOS1 or the mutant thereof, (ii) SHP2 or the mutant thereof, (iii) MEK or the mutant thereof, (iv) ERK or the mutant thereof, and (v) EGFR or the mutant thereof. FIG. 10 an example pathway depicting Ras signaling, showing a number of other signaling molecules involved in the Ras signaling pathway.

TABLE 1
Exemplary signaling molecules for cell proliferation

| No. | Name |
|---|---|
| 1 | SOS1 or a mutant thereof |
| 2 | SHP2 or a mutant thereof |
| 3 | SHC or a mutant thereof |
| 4 | GAB or a mutant thereof |
| 5 | GRB or a mutant thereof |
| 6 | JAK or a mutant thereof |
| 7 | A-RAF, B-RAF, C-RAF, or a mutant thereof |
| 8 | BRAF or a mutant thereof |
| 9 | MEK or a mutant thereof |
| 10 | ERK or a mutant thereof |
| 11 | PI3K or a mutant thereof |
| 12 | MAPK or a mutant thereof |
| 13 | EGFR or a mutant thereof |
| 14 | c-MET or a mutant thereof |
| 15 | ALK or a mutant thereof |
| 16 | FGFR1, FGFR-2, FGFR-3, FGFR-4 or a mutant |
| | thereof |
| 17 | BCR-ABL or a mutant thereof |
| 18 | ErbB2 (Her2) or a mutant thereof |
| 19 | AXL or a mutant thereof |
| 20 | NTRK1 or a mutant thereof |
| 21 | ROS1 or a mutant thereof |
| 22 | RET or a mutant thereof |
| 23 | MDM2 or a mutant thereof |
| 24 | mTOR or a mutant thereof |
| 25 | BET or a mutant thereof |
| 26 | IGF1, IGF2, or a mutant thereof |
| 27 | IGF1R or a mutant thereof |
| 28 | CDK9 or a mutant thereof |
| 29 | CDK4/6 |

In some embodiments, the downregulating is effectuated by contacting the cell with at least one inhibitor, such as a small molecule, nucleic acid agent, or a polypeptide (e.g., an endonuclease). In some cases, the cell can be contacted with at least about 1, 2, 3, 4, 5, 6, 7, 8, 9, 10, or more inhibitors. In some cases, the cell can be contacted with at most about 10, 9, 8, 7, 6, 5, 4, 3, 2, or 1 inhibitor. When using a plurality of inhibitors, the plurality of inhibitors can be capable of targeting (i) the same signaling molecule (e.g., a protein or a gene encoding the protein) or (ii) different signaling molecules. Contacting the cell with the at least one inhibitor can comprise (i) adding the at least one inhibitor to a fluid (e.g., liquid such as tissue, blood, media, etc.) that comprises the cell, (ii) administering the at least one inhibitor into the cell, and/or (ii) expressing the at least one inhibitor in the cell (e.g., by introducing a gene encoding the at least one inhibitor). The cell can be contacted by the at least one inhibitor for at least about 1, 2, 3, 4, 5, 6, 7, 8, 9, 10, or more times over a given period. The cell can be contacted by the at least one inhibitor for at least about 10, 9, 8, 7, 6, 5, 4, 3, 2, or 1 time over a given period. The cell can be contacted by the at least one inhibitor for at least about 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 16 hours, 20 hours, 24 hours, 2 days, 3 days, 4 days, 5 days, 6 days, 7 days, 8 days, 9 days, 10 days, 20 days, 30 days, or more. The cell can be contacted by at least one inhibitor for at most about 30 days, 20 days, 10 days, 9 days, 8 days, 7 days, 6 days, 5 days, 4 days, 3 days, 2 days, 24 hours, 20 hours, 16 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less. The contacting, as disclosed herein, can occur in vitro, ex vivo, or in vivo.

In some embodiments, the at least one inhibitor comprises a small molecule compound as further described herein. In some embodiments, the at least one inhibitor comprises a polypeptide, such as an endonuclease as disclosed herein, e.g., a Cas protein, a zinc finger nuclease (ZFN), a transcription activator-like effector nuclease (TALEN), a meganuclease, a recombinases, a flippase, a transposase, an Argonaute (Ago) protein, and a functional variant thereof. In some embodiments, the at least one inhibitor comprises a nucleic acid agent. A nucleic acid agent can comprise one or more members selected from the group consisting of an antisense oligonucleotide, a transfer RNA (tRNA), a small nuclear RNA (snRNA), a small interfering RNA (siRNA), a microRNA (miRNA), a small hairpin RNA (shRNA), a noncoding RNA (ncRNA), a pre-condensed DNA, an aptamer, a ribozyme, and a complex comprising a nucleic acid molecule and an endonuclease. In some cases, the nucleic acid agent is a nucleic acid molecule, and the nucleic acid molecule is sufficient to downregulate expression or activity of at least one signaling molecule (e.g., Kras G12D and one or more signaling molecules selected from Table 1). For example, the nucleic acid agent is a siRNA or a shRNA. In some cases, the nucleic acid agent is a complex comprising a nucleic acid molecule and an endonuclease as disclosed herein. The complex can be a covalently coupled complex or a non-covalently coupled (e.g., via hydrogen bonds and/or Van der Waals interaction) complex. For example, the nucleic acid agent is a complex comprising a Cas protein and a guide nucleic acid (e.g., a guide RNA) as disclosed herein.

In some embodiments, the cell is contacted with (i) an inhibitor against a Ras protein (e.g., Kras G12D) and (ii) at least one additional inhibitor against one or more signaling molecules selected from Table 1. The cell can be contacted with the inhibitor against the Ras protein and the at least one additional inhibitor either simultaneously or sequentially (e.g., contacting the cell with the inhibitor against the Ras protein before or after contacting the cell with the at least one additional inhibitor). For simultaneous contacting, the inhibitor against the Ras protein and the at least one additional inhibitor can be in the same composition (or formulation) or in different compositions (e.g., subjecting the cell to two different compositions at the same time). For sequential contacting, the inhibitor against the Ras protein and the at least one additional inhibitor can be in the same composition (e.g., a single composition exhibiting different release profiles of the inhibitor against the Ras protein and the at least one additional inhibitor) or in different compositions. For sequential contacting, a first contacting of the cell (e.g., with one of the inhibitor against the Ras protein and the at least one additional inhibitor) and a second contacting of the cell (e.g., with the other of the inhibitor against the Ras protein and the at least one additional inhibitor) can be separated by at least about 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 16 hours, 20 hours, 24 hours, or more. The first contacting and the second contacting can be separated by at most about 24 hours, 20 hours, 16 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less.

In some embodiments, a subject comprises the cell, and the at least one inhibitor is administered to the subject. The subject can be a mammal, such as an animal (e.g., a pig, horse, dog, etc.) or a human. In some embodiments, the subject comprising the cell is administered with (i) an inhibitor against a Ras protein (e.g., Kras G12D) and (ii) at least one additional inhibitor against one or more signaling molecules selected from Table 1. The subject can be administered with the inhibitor against the Ras protein and the at least one additional inhibitor either simultaneously or sequentially (e.g., administration of the inhibitor against the Ras protein before or after administration of the at least one additional inhibitor). For simultaneous administration, the inhibitor against the Ras protein and the at least one additional inhibitor can be in the same composition (or formulation) or in different compositions (e.g., administration of two different compositions at the same time to the same location or to different locations of the subject's body). For sequential administration, the inhibitor against the Ras protein and the at least one additional inhibitor can be administered in different compositions. For sequential administration, a first administration (e.g., administration of one of the inhibitor against the Ras protein and the at least one additional inhibitor) and a second administration (e.g., administration of the other of the inhibitor against the Ras protein and the at least one additional inhibitor) can be separated by at least about 1, 2, 3, 4, 5, 6, 7, 8, 9, 10, 20, 30, 40, 50, 60 minutes, 2, 3, 4, 5, 6, 7, 8, 9, 10, 11, 12, 16, 20, 24 hours, or more. The first administration and the second administration can be separated by at most about 24, 20, 16, 12, 11, 10, 9, 8, 7, 6, 5, 4, 3, 2 hours, 60, 50, 40, 30, 20, 10, 9, 8, 7, 6, 5, 4, 3, 2, 1 minute, or less.

In some embodiments, at least one additional inhibitor against one or more signaling molecules selected from Table 1 comprises a single inhibitor that is capable of inhibiting expression and/or activity of only a single signaling molecule selected from Table 1. In some embodiments, at least one additional inhibitor against one or more signaling molecules selected from Table 1 comprises a single inhibitor that is capable of inhibiting expression and/or activity of two or more different signaling molecules selected from Table 1. In some embodiments, at least one additional inhibitor against one or more signaling molecules selected from Table 1 comprises a plurality of different inhibitors capable of inhibiting expression and/or activity of a plurality of different signaling molecules selected from Table 1. In some embodiments, a first inhibitor against (a) Kras G12D and a second inhibitor against (b) one or more signaling molecules selected from Table 1 are operatively coupled to each other. In some examples, contacting of the cell with the first inhibitor effects (or activates) contacting of the cell with the second inhibitor, or vice versa. In some examples, expression of the first inhibitor in the cell (e.g., via activating a gene encoding at least a portion of the first inhibitor) effects (or activates) expression of the second inhibitor in the cell, or vice versa.

In some embodiments, the at least one inhibitor is capable of specifically binding to Kras G12D or a gene encoding Kras G12D. In some embodiments, the at least one inhibitor is capable of specifically binding to aspartic acid 12 residue of Kras. In some embodiments, the at least one inhibitor is capable of specifically binding to (i) one or more signaling molecules selected from Table 1 or (ii) one or more genes encoding the one or more signaling molecules selected from Table 1.

In some embodiments, cell signaling (e.g., cell proliferation signaling) in a cell can be downregulated (e.g., inhibited) by at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300- fold, 400-fold, 500-fold, 600-fold, 700-fold, 800-fold, 900-fold, 1000-fold, 2000-fold, 3000-fold, 4000-fold, 5000-fold, or more as compared to a control cell. The cell signaling in the cell can be downregulated by at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less as compared to the control cell. The downregulation of cell signaling in the cell can be maintained (or prolonged) for at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40- fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more longer as compared to a control cell. The downregulation of cell signaling in the cell can be maintained for at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less longer as compared to the control cell.

In some embodiments, cell proliferation signaling in a cell can be downregulated (e.g., inhibited) by at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control cell.

The cell proliferation signaling in the cell can be downregulated by at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less as compared to the control cell. The downregulation of cell proliferation signaling in the cell can be maintained (or prolonged) for at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more longer as compared to a control cell. The downregulation of cell proliferation signaling in the cell can be maintained for at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less longer as compared to the control cell.

In some embodiments, the expression and/or activity of (i) a Ras protein (e.g., Kras G12D) and that of (ii) one or more signaling molecules selected from Table 1 in a cell can be downregulated by at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control cell. The expression and/or activity of (i) the Ras protein (e.g., Kras G12D) and that of (ii) the one or more signaling molecules selected from Table 1 in the cell can be downregulated by at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2- fold, 0.1-fold, or less as compared to the control cell. The downregulation of expression and/or activity of (i) the Ras protein (e.g., Kras G12D) and that of (ii) the one or more signaling molecules selected from Table 1 in the cell can be maintained (or prolonged) for at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more longer as compared to a control cell. The downregulation of expression and/or activity of (i) the Ras protein (e.g., Kras G12D) and that of (ii) the one or more signaling molecules selected from Table 1 in the cell can be maintained for at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1- fold, 0.9-fold, 0.8-fold, 0.7-fold, 0.6-fold, 0.5-fold, 0.4-fold, 0.3-fold, 0.2-fold, 0.1-fold, or less longer as compared to the control cell.

In some embodiments, downregulating expression and/or activity of (a) Kras G12D and that of (b) one or more signaling molecules selected from Table 1 reduces Ras signaling output in a cell. In some embodiments, the reduction in Ras signaling output is evidenced by one or more members of the following: (i) an increase in steady state level of GDP-bound Ras protein; (ii) a reduction of phosphorylated AKTs473, (iii) a reduction of phosphorylated ERKT202/y204, (iv) a reduction of phosphorylated S6S235/236, and (v) reduction (e.g., inhibition) of cell growth of the cell (e.g., a Ras-driven tumor cell, such as that derived from a tumor cell line). In some cases, the reduction in Ras signaling output can be evidenced by two or more members of (i)-(v). In some cases, the reduction in Ras signaling output can be evidenced by three or more members of (i)-(v). In some cases, the reduction in Ras signaling output can be evidenced by four or more members of (i)-(v). In some cases, the reduction in Ras signaling output can be evidenced by all of (i)-(v). A GDP-bound Ras protein (e.g., a GDP-bound Kras G12D) may exhibit a lower degree of signaling activity (e.g., cell proliferation signaling activity) as compared to a GTP-bound Ras protein (e.g., a GTP-bound Kras G12D).

The reduction in Ras signaling output can be evidenced by an increase in steady state level of GDP-bound Ras protein as compared to (i) a steady state level of GDP-bound Ras protein in a control cell and/or (ii) control Ras proteins. A control Ras protein, as described herein, can be a Ras protein (e.g., wildtype or mutated) that is not contacted by an inhibitor against the present disclosure.

The increase in steady state level of GDP-bound Ras protein can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to (i) that in a control cell and/or (ii) the control Ras proteins. The increase in steady state level of GDP-bound Ras protein can be at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less as compared to (i) that in a control cell and/or (ii) the control Ras proteins.

The reduction in Ras signaling output can be evidenced by a reduction of phosphorylated AKTs473 as compared to phosphorylation of AKTs473 in (i) a control cell and/or (ii) in the presence of control Ras proteins. The reduction of phosphorylated AKTs473 can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the phosphorylation of AKTs473 in (i) a control cell and/or (ii) in the presence of control Ras proteins.

The reduction of phosphorylated AKTs473 can be at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less as compared to the phosphorylation of AKTs473 in (i) a control cell and/or (ii) in the presence of control Ras proteins.

The reduction in Ras signaling output can be evidenced by a reduction of phosphorylated ERKT202/y204 as compared to phosphorylation of ERKT202/y204 in (i) a control cell and/or (ii) in the presence of control Ras proteins. The reduction of phosphorylated ERKT202/y204 can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the phosphorylation of ERKT202/y204 in (i) a control cell and/or (ii) in the presence of control Ras proteins. The reduction of phosphorylated ERKT202/y204 can be at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1- fold, 0.9-fold, 0.8-fold, 0.7-fold, 0.6-fold, 0.5-fold, 0.4-fold, 0.3-fold, 0.2-fold, 0.1-fold, or less as compared to the phosphorylation of ERKT202/y204 in (i) a control cell and/or (ii) in the presence of control Ras proteins.

The reduction in Ras signaling output can be evidenced by a reduction of phosphorylated S6S235/236 as compared to phosphorylation of S6S235/236 in (i) a control cell and/or (ii) in the presence of control Ras proteins. The reduction of phosphorylated S6S235/236 can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the phosphorylation of S6S235/236 in (i) a control cell and/or (ii) in the presence of control Ras proteins. The reduction of phosphorylated S6S235/236 can be at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8- fold, 0.7-fold, 0.6-fold, 0.5-fold, 0.4-fold, 0.3-fold, 0.2-fold, 0.1-fold, or less as compared to the phosphorylation of S6S235/236 in (i) a control cell and/or (ii) in the presence of control Ras proteins.

The reduction in Ras signaling output can be evidenced by a reduction (e.g., inhibition) of cell growth of Ras-driven tumor cells as compared to a control cell (e.g., a control tumor cell). The reduction of cell growth of Ras-driven tumor cells can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control cell. The reduction of cell growth of Ras-driven tumor cells can be at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less as compared to a control cell.

In some embodiments, downregulating expression or activity of (a) Kras G12D and that of (b) one or more signaling molecules selected from Table 1 synergistically yields a greater degree of inhibition of cell proliferation in the cell as compared to (1) an individual degree of inhibition of cell proliferation via downregulating expression or activity of one of (a) and (b) (e.g., (a) alone or (b) alone) and/or (2) a sum of the individual degrees of inhibition of cell proliferation thereof. In some examples, a degree of inhibition of cell proliferation in the cell by downregulating expression or activity of both (a) and (b) is at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8- fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more greater than (1) an individual degree of inhibition of cell proliferation in a control cell by downregulating expression or activity of one of (a) and (b), alone and/or (2) a sum of the individual degrees of inhibition of cell proliferation thereof. In some examples, a degree of inhibition of cell proliferation in the cell by downregulating expression or activity of both (a) and (b) is at least about at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less greater than (1) an individual degree of inhibition of cell proliferation in a control cell by downregulating expression or activity of one of (a) and (b), alone and/or (2) a sum of the individual degrees of inhibition of cell proliferation thereof. The inhibition of cell proliferation in the cell by downregulating expression or activity of both (a) and (b) can be maintained (or prolonged) for at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more longer as compared to (1) individual inhibition of cell proliferation in a control cell by downregulating expression or activity of one of (a) and (b), alone and/or (2) a sum of the individual inhibition of cell proliferation thereof. The inhibition of cell proliferation in the cell by downregulating expression or activity of both (a) and (b) can be maintained (or prolonged) for at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less longer as compared to (1) individual inhibition of cell proliferation in a control cell by downregulating expression or activity of one of (a) and (b), alone or (2) a sum of the individual inhibition of cell proliferation thereof.

In some embodiments, a control or a control cell as disclosed herein can be an alternative sample or subject used in an experiment for comparison purpose. In some cases, a control cell can be a cell that does not comprise Kras G12D mutation. In some cases, a control cell can be a cell that is not subjected to any treatment to inhibit cell signaling (e.g., cell proliferation signaling). In some cases, a control cell can be a cell that is not subjected to any treatment to downregulate expression and/or activity of (a) Kras G12D and that of (b) one or more signaling molecules selected from Table 1. In some cases, a control cell can be a cell that is subjected to only one of: (a) an inhibitor against Kras G12D and (b) at least one additional inhibitor against one or more signaling molecules selected from Table 1.

A synergistic effect of a combination treatment comprising a plurality of agents, as disclosed herein, can be characterized by a synergistic value as ascertained by an “excess over Bliss independence” or “BLISS” independence criterion. The BLISS independence criterion can be used to screen for candidate drug combinations. The criterion can compare the observed combination response with the predicted combination response, which predicted combination response is obtained based on the assumption that there is no effect from drug-drug interactions. The combination effect can be determined to be synergistic when the observed combination response is greater than the predicted combination response (e.g., greater by a threshold value). To determine a synergistic value of a combination treatment comprising inhibitor (a) (e.g., a Kras G12D inhibitor) and inhibitor (b) (e.g., at least one inhibitor of a signaling molecule selected from Table 1) for inducing growth inhibition of target cells (e.g., cancer cells), the BLISS independence criterion can utilize the following equation:

Y_{AB,O}−Y_{AB,P} (1)

wherein:
Y_{AB,O} is observed percentage growth inhibition of the target cells by the combination comprising (a) at dose A and (b) at dose B; and
Y_{AB,P} is predicted percentage growth inhibition of the target cells by the combination comprising (a) at the dose A, and (b) at the dose B, wherein:

Y_{AB,P}=Y_{A}+Y_{B}−Y_{A}Y_{B} (2)

wherein further:
Y_{A} is observed percentage growth inhibition of the target cells by (a) alone at the dose A;
Y_{B} is observed percentage growth inhibition of the target cells by (b) alone at the dose, B; and
Y_{A}Y_{B} is product of Y_{A} and Y_{B}.

The observed combined percentage inhibition Y_{AB,O} is compared with the predicted percentage growth inhibition Y_{AB,P}, in accordance with equation (1). The comparison can determine whether the combination treatment promotes a synergistic effect, an additive effect, or an antagonistic effect, as summarized in equation (3). When Y_{AB,O}>Y_{AB,P}, the combination treatment can be determined to be more efficacious than expected (e.g., a synergistic effect). When Y_{AB,O}<Y_{AB,P}, the combination treatment can be determined to be worse than expected (e.g., an antagonistic effect). When Y_{AB,O}=Y_{AB,P}, the combination treatment can be determined to be substantially the same as a simple addition of two separate drugs (e.g., independent effects, or an additive effect).

$${Y}_{AB , P} \{ \begin{matrix}> {Y}_{AB , P} & Synergy \\ = {Y}_{AB , P} & Independent \\ < {Y}_{AB , P} & Antagonism\end{matrix} \qquad ( 3 )$$

When using the BLISS independence criterion, the percentage growth inhibition of the target cells can be provided based on a percentage scale (e.g., between about 0% to about 100%) or a fractional scale (e.g., between about 0 to 1). For example, a 75% growth inhibition of the target cells can be expressed as 0.75 for purposes of analysis in accordance with the BLISS independence criterion. For example, with the fractional scale is used, the difference between the observed combined percentage inhibition Y_{AB,O} and the predicted percentage growth inhibition Y_{AB,P}, in accordance with equation (1) (e.g., based on one or more in vitro experiments), can be determined to be additive (or antagonistic) when the difference is less than or equal to zero. Such difference can be determined to be synergistic when the difference is greater than zero. Here, the synergistic effect can be divided into a plurality of sub-ranges, e.g., a first synergistic sub-range having the difference between about 0.05 and about 0.1 (e.g., mild synergy), a second synergistic sub-range having the difference between about 0.1 and about 0.2 (e.g., moderate synergy), and a third synergistic sub-range having the difference greater than or equal to 0.2 (e.g., robust synergy).

In some embodiments, the combination treatment comprising a plurality of agents (e.g., a Kras G12D inhibitor and at least one inhibitor of a signaling molecule selected from Table 1), as disclosed herein, can be utilized to reduce growth or proliferation of target cells, such as cancer cells, in vitro or in vivo. The therapeutic efficacy of the combination treatment can be characterized by a synergistic value of about 0.01 to about 0.5, as ascertained by the Bliss independent criterion. The therapeutic efficacy of the combination treatment can be characterized by a synergistic value of at least about 0.01, as ascertained by the Bliss independent criterion. The therapeutic efficacy of the combination treatment can be characterized by a synergistic value of at most about 0.5, as ascertained by the Bliss independent criterion. The therapeutic efficacy of the combination treatment can be characterized by a synergistic value of about 0.01 to about 0.05, about 0.01 to about 0.07, about 0.01 to about 0.1, about 0.01 to about 0.12, about 0.01 to about 0.15, about 0.01 to about 0.17, about 0.01 to about 0.2, about 0.01 to about 0.25, about 0.01 to about 0.3, about 0.01 to about 0.4, about 0.01 to about 0.5, about 0.05 to about 0.07, about 0.05 to about 0.1, about 0.05 to about 0.12, about 0.05 to about 0.15, about 0.05 to about 0.17, about 0.05 to about 0.2, about 0.05 to about 0.25, about 0.05 to about 0.3, about 0.05 to about 0.4, about 0.05 to about 0.5, about 0.07 to about 0.1, about 0.07 to about 0.12, about 0.07 to about 0.15, about 0.07 to about 0.17, about 0.07 to about 0.2, about 0.07 to about 0.25, about 0.07 to about 0.3, about 0.07 to about 0.4, about 0.07 to about 0.5, about 0.1 to about 0.12, about 0.1 to about 0.15, about 0.1 to about 0.17, about 0.1 to about 0.2, about 0.1 to about 0.25, about 0.1 to about 0.3, about 0.1 to about 0.4, about 0.1 to about 0.5, about 0.12 to about 0.15, about 0.12 to about 0.17, about 0.12 to about 0.2, about 0.12 to about 0.25, about 0.12 to about 0.3, about 0.12 to about 0.4, about 0.12 to about 0.5, about 0.15 to about 0.17, about 0.15 to about 0.2, about 0.15 to about 0.25, about 0.15 to about 0.3, about 0.15 to about 0.4, about 0.15 to about 0.5, about 0.17 to about 0.2, about 0.17 to about 0.25, about 0.17 to about 0.3, about 0.17 to about 0.4, about 0.17 to about 0.5, about 0.2 to about 0.25, about 0.2 to about 0.3, about 0.2 to about 0.4, about 0.2 to about 0.5, about 0.25 to about 0.3, about 0.25 to about 0.4, about 0.25 to about 0.5, about 0.3 to about 0.4, about 0.3 to about 0.5, or about 0.4 to about 0.5, as ascertained by the Bliss independent criterion. The therapeutic efficacy of the combination treatment can be characterized by a synergistic value of about 0.01, about 0.05, about 0.07, about 0.1, about 0.12, about 0.15, about 0.17, about 0.2, about 0.25, about 0.3, about 0.4, or about 0.5, as ascertained by the Bliss independent criterion.

A unit dosage of an agent as disclosed herein (e.g., one or more inhibitors of the combination treatment for in vivo administration) can comprise about 0.01 mg to about 5,000 mg. A unit dosage of an agent can comprise at least about 0.01 mg. A unit dosage of an agent can comprise at most about 5,000 mg. A unit dosage of an agent can comprise about 0.01 mg to about 0.05 mg, about 0.01 mg to about 0.1 mg, about 0.01 mg to about 0.5 mg, about 0.01 mg to about 1 mg, about 0.01 mg to about 5 mg, about 0.01 mg to about 10 mg, about 0.01 mg to about 50 mg, about 0.01 mg to about 100 mg, about 0.01 mg to about 500 mg, about 0.01 mg to about 1,000 mg, about 0.01 mg to about 5,000 mg, about 0.05 mg to about 0.1 mg, about 0.05 mg to about 0.5 mg, about 0.05 mg to about 1 mg, about 0.05 mg to about 5 mg, about 0.05 mg to about 10 mg, about 0.05 mg to about 50 mg, about 0.05 mg to about 100 mg, about 0.05 mg to about 500 mg, about 0.05 mg to about 1,000 mg, about 0.05 mg to about 5,000 mg, about 0.1 mg to about 0.5 mg, about 0.1 mg to about 1 mg, about 0.1 mg to about 5 mg, about 0.1 mg to about 10 mg, about 0.1 mg to about 50 mg, about 0.1 mg to about 100 mg, about 0.1 mg to about 500 mg, about 0.1 mg to about 1,000 mg, about 0.1 mg to about 5,000 mg, about 0.5 mg to about 1 mg, about 0.5 mg to about 5 mg, about 0.5 mg to about 10 mg, about 0.5 mg to about 50 mg, about 0.5 mg to about 100 mg, about 0.5 mg to about 500 mg, about 0.5 mg to about 1,000 mg, about 0.5 mg to about 5,000 mg, about 1 mg to about 5 mg, about 1 mg to about 10 mg, about 1 mg to about 50 mg, about 1 mg to about 100 mg, about 1 mg to about 500 mg, about 1 mg to about 1,000 mg, about 1 mg to about 5,000 mg, about 5 mg to about 10 mg, about 5 mg to about 50 mg, about 5 mg to about 100 mg, about 5 mg to about 500 mg, about 5 mg to about 1,000 mg, about 5 mg to about 5,000 mg, about 10 mg to about 50 mg, about 10 mg to about 100 mg, about 10 mg to about 500 mg, about 10 mg to about 1,000 mg, about 10 mg to about 5,000 mg, about 50 mg to about 100 mg, about 50 mg to about 500 mg, about 50 mg to about 1,000 mg, about 50 mg to about 5,000 mg, about 100 mg to about 500 mg, about 100 mg to about 1,000 mg, about 100 mg to about 5,000 mg, about 500 mg to about 1,000 mg, about 500 mg to about 5,000 mg, or about 1,000 mg to about 5,000 mg. A unit dosage of an agent can comprise about 0.01 mg, about 0.05 mg, about 0.1 mg, about 0.5 mg, about 1 mg, about 5 mg, about 10 mg, about 50 mg, about 100 mg, about 500 mg, about 1,000 mg, or about 5,000 mg.

A unit dosage of an agent as disclosed herein can yield a therapeutic concentration (e.g., a non-clinical concentration such as that in a medium for in vitro assays, a clinical blood concentration in vivo, etc.) of about 0.1 micromolar to about 100 micromolar. A unit dosage of an agent as disclosed herein can yield a therapeutic concentration of at least about 0.1 micromolar. A unit dosage of an agent as disclosed herein can yield a therapeutic concentration of at most about 100 micromolar. A unit dosage of an agent as disclosed herein can yield a therapeutic concentration of about 0.1 micromolar to about 0.2 micromolar, about 0.1 micromolar to about 0.5 micromolar, about 0.1 micromolar to about 1 micromolar, about 0.1 micromolar to about 2 micromolar, about 0.1 micromolar to about 5 micromolar, about 0.1 micromolar to about 10 micromolar, about 0.1 micromolar to about 20 micromolar, about 0.1 micromolar to about 50 micromolar, about 0.1 micromolar to about 100 micromolar, about 0.2 micromolar to about 0.5 micromolar, about 0.2 micromolar to about 1 micromolar, about 0.2 micromolar to about 2 micromolar, about 0.2 micromolar to about 5 micromolar, about 0.2 micromolar to about 10 micromolar, about 0.2 micromolar to about 20 micromolar, about 0.2 micromolar to about 50 micromolar, about 0.2 micromolar to about 100 micromolar, about 0.5 micromolar to about 1 micromolar, about 0.5 micromolar to about 2 micromolar, about 0.5 micromolar to about 5 micromolar, about 0.5 micromolar to about 10 micromolar, about 0.5 micromolar to about 20 micromolar, about 0.5 micromolar to about 50 micromolar, about 0.5 micromolar to about 100 micromolar, about 1 micromolar to about 2 micromolar, about 1 micromolar to about 5 micromolar, about 1 micromolar to about 10 micromolar, about 1 micromolar to about 20 micromolar, about 1 micromolar to about 50 micromolar, about 1 micromolar to about 100 micromolar, about 2 micromolar to about 5 micromolar, about 2 micromolar to about 10 micromolar, about 2 micromolar to about 20 micromolar, about 2 micromolar to about 50 micromolar, about 2 micromolar to about 100 micromolar, about 5 micromolar to about 10 micromolar, about 5 micromolar to about 20 micromolar, about 5 micromolar to about 50 micromolar, about 5 micromolar to about 100 micromolar, about 10 micromolar to about 20 micromolar, about 10 micromolar to about 50 micromolar, about 10 micromolar to about 100 micromolar, about 20 micromolar to about 50 micromolar, about 20 micromolar to about 100 micromolar, or about 50 micromolar to about 100 micromolar. A unit dosage of an agent as disclosed herein can yield a therapeutic concentration of about 0.1 micromolar, about 0.2 micromolar, about 0.5 micromolar, about 1 micromolar, about 2 micromolar, about 5 micromolar, about 10 micromolar, about 20 micromolar, about 50 micromolar, or about 100 micromolar.

A unit dosage of an agent as disclosed herein can yield a therapeutic concentration (e.g., a non-clinical concentration such as that in a medium for in vitro assays, a clinical blood concentration in vivo, etc.) of about 0.1 nanomolar to about 1,000 nanomolar. A unit dosage of an agent as disclosed herein can yield a therapeutic concentration of at least about 0.1 nanomolar. A unit dosage of an agent as disclosed herein can yield a therapeutic concentration of at most about 1,000 nanomolar. A unit dosage of an agent as disclosed herein can yield a therapeutic concentration of about 0.1 nanomolar to about 0.2 nanomolar, about 0.1 nanomolar to about 0.5 nanomolar, about 0.1 nanomolar to about 1 nanomolar, about 0.1 nanomolar to about 2 nanomolar, about 0.1 nanomolar to about 5 nanomolar, about 0.1 nanomolar to about 10 nanomolar, about 0.1 nanomolar to about 20 nanomolar, about 0.1 nanomolar to about 50 nanomolar, about 0.1 nanomolar to about 100 nanomolar, about 0.1 nanomolar to about 200 nanomolar, about 0.1 nanomolar to about 1,000 nanomolar, about 0.2 nanomolar to about 0.5 nanomolar, about 0.2 nanomolar to about 1 nanomolar, about 0.2 nanomolar to about 2 nanomolar, about 0.2 nanomolar to about 5 nanomolar, about 0.2 nanomolar to about 10 nanomolar, about 0.2 nanomolar to about 20 nanomolar, about 0.2 nanomolar to about 50 nanomolar, about 0.2 nanomolar to about 100 nanomolar, about 0.2 nanomolar to about 200 nanomolar, about 0.2 nanomolar to about 1,000 nanomolar, about 0.5 nanomolar to about 1 nanomolar, about 0.5 nanomolar to about 2 nanomolar, about 0.5 nanomolar to about 5 nanomolar, about 0.5 nanomolar to about 10 nanomolar, about 0.5 nanomolar to about 20 nanomolar, about 0.5 nanomolar to about 50 nanomolar, about 0.5 nanomolar to about 100 nanomolar, about 0.5 nanomolar to about 200 nanomolar, about 0.5 nanomolar to about 1,000 nanomolar, about 1 nanomolar to about 2 nanomolar, about 1 nanomolar to about 5 nanomolar, about 1 nanomolar to about 10 nanomolar, about 1 nanomolar to about 20 nanomolar, about 1 nanomolar to about 50 nanomolar, about 1 nanomolar to about 100 nanomolar, about 1 nanomolar to about 200 nanomolar, about 1 nanomolar to about 1,000 nanomolar, about 2 nanomolar to about 5 nanomolar, about 2 nanomolar to about 10 nanomolar, about 2 nanomolar to about 20 nanomolar, about 2 nanomolar to about 50 nanomolar, about 2 nanomolar to about 100 nanomolar, about 2 nanomolar to about 200 nanomolar, about 2 nanomolar to about 1,000 nanomolar, about 5 nanomolar to about 10 nanomolar, about 5 nanomolar to about 20 nanomolar, about 5 nanomolar to about 50 nanomolar, about 5 nanomolar to about 100 nanomolar, about 5 nanomolar to about 200 nanomolar, about 5 nanomolar to about 1,000 nanomolar, about 10 nanomolar to about 20 nanomolar, about 10 nanomolar to about 50 nanomolar, about 10 nanomolar to about 100 nanomolar, about 10 nanomolar to about 200 nanomolar, about 10 nanomolar to about 1,000 nanomolar, about 20 nanomolar to about 50 nanomolar, about 20 nanomolar to about 100 nanomolar, about 20 nanomolar to about 200 nanomolar, about 20 nanomolar to about 1,000 nanomolar, about 50 nanomolar to about 100 nanomolar, about 50 nanomolar to about 200 nanomolar, about 50 nanomolar to about 1,000 nanomolar, about 100 nanomolar to about 200 nanomolar, about 100 nanomolar to about 1,000 nanomolar, or about 200 nanomolar to about 1,000 nanomolar. A unit dosage of an agent as disclosed herein can yield a therapeutic concentration of about 0.1 nanomolar, about 0.2 nanomolar, about 0.5 nanomolar, about 1 nanomolar, about 2 nanomolar, about 5 nanomolar, about 10 nanomolar, about 20 nanomolar, about 50 nanomolar, about 100 nanomolar, about 200 nanomolar, or about 1,000 nanomolar.

When an agent as disclosed herein (e.g., a Kras G12D inhibitor) is tested in vitro, the in vitro unit dosage (or concentration) of the agent can be selected to correspond to a clinically relevant unit dosage of the agent, e.g., for administration to a human subject.

The in vitro concentration of the agent can be determined based on one or more pharmacokinetic (PK) parameters obtained from in vivo PK data of the agent (e.g., human PK data of the agent). The in vivo PK data can be associated with a clinically recommended or approved dose (e.g., the highest recommended dose) of the agent. In some cases, a clinically relevant concentration of the agent for in vitro assays can be defined by one or more PK parameters from in vivo PK data of the agent, comprising (i) the maximum plasma concentration (Cₘₐₓ), (ii) the minimum plasma concentration (Cₘᵢₙ), (iii) the average plasma concentration (C_{avg}), (iv) the integrated area under the curve (AUC), and/or (v) the time to reach Cₘₐₓ (tₘₐₓ). For example, an agent (e.g., a drug inhibitor) as disclosed herein can be tested in vitro at a concentration range (e.g., between about 1 micromolar to 10 micromolar) that encompasses Cₘₐₓ of the agent observed when used at the highest recommended dose of the agent (e.g., 5 micromolar). Inclusion of drug concentrations or exposures that are relevant to those observed in clinical use can improve translation of nonclinical mechanism of action findings and/or nonclinical outcomes into potentially relevant clinical effects. Without wishing to be bound by theory, the working concentration or concentration range in vitro of any one of the therapeutic agents disclosed herein may be similar or substantially the same as the clinically achievable and/or preferred exposure of the therapeutic agent(s) to a human subject.

Unit dosages, PK parameters, and clinical outcomes of various drug inhibitors, as disclosed and utilized herein, are described in, for example, (1) Tan et al., J Clin Oncol. 2004 Aug. 1; 22(15):3080-90 (PMID:15284258); (2) Wind et al., Clin Pharmacokinet. 2017 March; 56(3):235-250 (PMID: 27470518); (3) Tan et al., Clin Cancer Res. 2006 Nov. 1; 12(21):6517-22 (PMID: 17065274); (4) Ouellet et al., Cancer Chemother Pharmacol. 2016 April; 77(4):807-17 (PMID: 26940938); (5) Groenland et al., Clin Pharmacokinet. 2020 December; 59(12):1501-1520 (PMID: 33029704); and (6) Jurie et al., JAMA Oncol. 2019 Feb. 1; 5(2):e184475 (PMID: 30543347), each of which is entirely incorporated herein by reference.

Similarly, a duration of exposure of one or more cells to an agent in in vitro assays can be selected to correspond to a clinically relevant duration of exposure of the agent to a subject in need thereof.

The combination treatment can comprise a first inhibitor (e.g., a Kras G12D inhibitor) and a second inhibitor (e.g., at least one inhibitor of the signaling molecule selected from Table 1), and a unit dosage of the first inhibitor and a unit dosage of the second inhibitor can be the same. Alternatively, a unit dosage of the first inhibitor and a unit dosage of the second inhibitor can be different.

The unit dosage of the first inhibitor (UD1) and the unit dosage of the second inhibitor (UD2) can be characterized by a ratio (e.g., a weight ratio or a molar ratio) of about 100:1 to about 1:100 (UD1:UD2). The ratio (UD1:UD2) can be at least about 100:1, 80:1, 60:1, 50:1, 40:1, 20:1, 10:1, 8:1, 6:1, 5:1, 4:1, 2:1, 1:1, 1:2, 1:4, 1:5, 1:6, 1:8, 1:10, 1:20, 1:40, 1:50, 1:60, 1:80, or 1:100. The ratio (UD1:UD2) can be at most about 100:1, 80:1, 60:1, 50:1, 40:1, 20:1, 10:1, 8:1, 6:1, 5:1, 4:1, 2:1, 1:1, 1:2, 1:4, 1:5, 1:6, 1:8, 1:10, 1:20, 1:40, 1:50, 1:60, 1:80, or 1:100.

The combination treatment as disclosed herein can utilize one or more agents (e.g., one or more drugs), which may be sub-optimal when used alone, to yield a desired therapeutic efficacy by promoting a synergistic effect of the plurality of agents. The combination, when tested in vitro, ex vivo, or in vivo, can yield a comparable degree of therapeutic efficacy (e.g., cancer cell growth inhibition, tumor clearance, etc.) to that mediated by a control agent (e.g., a control drug) that is more potent that any of the agents of the combination. In some cases, the control agent can be an agent that is different from (e.g., mechanistically, molecularly, etc.) and more therapeutically effective than any one of the agents of the combination. In some cases, the control agent can be one of the drugs of the combination, but at a higher unit dosage. A comparable degree of therapeutic efficacy (or outcome) of the combination treatment as disclosed herein can be at least about 80%, 82%, 84%, 85%, 86%, 88%, 90%, 91%, 92%, 93%, 94%, 95%, 96%, 97%, 98%, 99%, 100%, 101%, 102%, 103%, 104%, 105%, 110%, 115%, 120%, 150%, 200%, or more with respect to a therapeutic efficacy of the control agent (e.g., a therapeutic efficacy of a clinically approved drug).

In some embodiments, the combination treatment as disclosed herein can comprise (i) an inhibitor against a first target (e.g., Kras G12D protein or a gene encoding thereof) and (ii) an inhibitor against a second target (e.g., a signaling molecule from Table 1 or a gene encoding thereof). The therapeutic efficacy of the combination treatment (e.g., as ascertained by a percentage of growth inhibition of cancer cells, or by a degree of tumor clearance) can be comparable to a therapeutic efficacy achieved by a more potent control inhibitor of the first target or the second target. A higher potency as disclosed herein can be characterized by exhibiting a lower IC50 value. When ascertained by an in vitro growth inhibition assay utilizing the cancer cells, a higher potency as disclosed herein can be characterized by exhibiting a lower cellular IC50 value. A cellular IC50 value of the more potent control inhibitor can be at least 0.1, 0.2, 0.3, 0.4, 0.5, 0.6, 0.7, 0.8, 0.9, 1, 1.2, 1.5, 2, 3, 4, 5, or more orders of magnitude lower than a cellular IC50 value of the combination's inhibitor against the first target or the second target. The cellular IC50 value of the more potent control inhibitor can be lower than the cellular IC50 value of the combination's inhibitor against the first target or the second target by at least about 1 nanomolar, 2 nanomolar, 3 nanomolar, 4 nanomolar, 5 nanomolar, 10 nanomolar, 20 nanomolar, 30 nanomolar, 40 nanomolar, 50 nanomolar, 100 nanomolar, 200 nanomolar, 300 nanomolar, 400 nanomolar, 500 nanomolar, 1 micromolar, 2 micromolar, 3 micromolar, 4 micromolar, 5 micromolar, 10 micromolar, 20 micromolar, 30 micromolar, 40 micromolar, 50 micromolar, 100 micromolar, 200 micromolar, 300 micromolar, 400 micromolar, 500 micromolar, or more. The cellular IC50 value of the more potent control inhibitor can be lower than the cellular IC50 value of the combination's inhibitor against the first target or the second target by at most about 500 micromolar, 400 micromolar, 300 micromolar, 200 micromolar, 100 micromolar, 50 micromolar, 40 micromolar, 30 micromolar, 20 micromolar, 10 micromolar, 5 micromolar, 4 micromolar, 3 micromolar, 2 micromolar, 1 micromolar, 500 nanomolar, 400 nanomolar, 300 nanomolar, 200 nanomolar, 100 nanomolar, 50 nanomolar, 40 nanomolar, 30 nanomolar, 20 nanomolar, 10 nanomolar, 5 nanomolar, 4 nanomolar, 3 nanomolar, 2 nanomolar, 1 nanomolar, or less.

The cellular IC50 value of the more potent control inhibitor can be less than about 10 nanomolar, 9 nanomolar, 8 nanomolar, 7 nanomolar, 6 nanomolar, 5 nanomolar, 4 nanomolar, 3 nanomolar, 2 nanomolar, 1 nanomolar, 0.9 nanomolar, 0.8 nanomolar, 0.7 nanomolar, 0.6 nanomolar, 0.5 nanomolar, 0.4 nanomolar, 0.3 nanomolar, 0.2 nanomolar, 0.1 nanomolar, or less. The cellular IC50 value of the combination's inhibitor against the first target or the second target can be about 10 nanomolar to about 5,000 nanomolar. The cellular IC50 value of the combination's inhibitor against the first target or the second target can be at least about 10 nanomolar. The cellular IC50 value of the combination's inhibitor against the first target or the second target can be at most about 5,000 nanomolar. The cellular IC50 value of the combination's inhibitor against the first target or the second target can be about 10 nanomolar to about 20 nanomolar, about 10 nanomolar to about 50 nanomolar, about 10 nanomolar to about 70 nanomolar, about 10 nanomolar to about 100 nanomolar, about 10 nanomolar to about 200 nanomolar, about 10 nanomolar to about 500 nanomolar, about 10 nanomolar to about 700 nanomolar, about 10 nanomolar to about 1,000 nanomolar, about 10 nanomolar to about 2,000 nanomolar, about 10 nanomolar to about 5,000 nanomolar, about 20 nanomolar to about 50 nanomolar, about 20 nanomolar to about 70 nanomolar, about 20 nanomolar to about 100 nanomolar, about 20 nanomolar to about 200 nanomolar, about 20 nanomolar to about 500 nanomolar, about 20 nanomolar to about 700 nanomolar, about 20 nanomolar to about 1,000 nanomolar, about 20 nanomolar to about 2,000 nanomolar, about 20 nanomolar to about 5,000 nanomolar, about 50 nanomolar to about 70 nanomolar, about 50 nanomolar to about 100 nanomolar, about 50 nanomolar to about 200 nanomolar, about 50 nanomolar to about 500 nanomolar, about 50 nanomolar to about 700 nanomolar, about 50 nanomolar to about 1,000 nanomolar, about 50 nanomolar to about 2,000 nanomolar, about 50 nanomolar to about 5,000 nanomolar, about 70 nanomolar to about 100 nanomolar, about 70 nanomolar to about 200 nanomolar, about 70 nanomolar to about 500 nanomolar, about 70 nanomolar to about 700 nanomolar, about 70 nanomolar to about 1,000 nanomolar, about 70 nanomolar to about 2,000 nanomolar, about 70 nanomolar to about 5,000 nanomolar, about 100 nanomolar to about 200 nanomolar, about 100 nanomolar to about 500 nanomolar, about 100 nanomolar to about 700 nanomolar, about 100 nanomolar to about 1,000 nanomolar, about 100 nanomolar to about 2,000 nanomolar, about 100 nanomolar to about 5,000 nanomolar, about 200 nanomolar to about 500 nanomolar, about 200 nanomolar to about 700 nanomolar, about 200 nanomolar to about 1,000 nanomolar, about 200 nanomolar to about 2,000 nanomolar, about 200 nanomolar to about 5,000 nanomolar, about 500 nanomolar to about 700 nanomolar, about 500 nanomolar to about 1,000 nanomolar, about 500 nanomolar to about 2,000 nanomolar, about 500 nanomolar to about 5,000 nanomolar, about 700 nanomolar to about 1,000 nanomolar, about 700 nanomolar to about 2,000 nanomolar, about 700 nanomolar to about 5,000 nanomolar, about 1,000 nanomolar to about 2,000 nanomolar, about 1,000 nanomolar to about 5,000 nanomolar, or about 2,000 nanomolar to about 5,000 nanomolar. The cellular IC50 value of the combination's inhibitor against the first target or the second target can be about 10 nanomolar, about 20 nanomolar, about 50 nanomolar, about 70 nanomolar, about 100 nanomolar, about 200 nanomolar, about 500 nanomolar, about 700 nanomolar, about 1,000 nanomolar, about 2,000 nanomolar, or about 5,000 nanomolar.

In some embodiments, the combination treatment as disclosed herein can comprise (i) an inhibitor against a first target (e.g., Kras G12D protein or a gene encoding thereof) and (ii) an inhibitor against a second target (e.g., a signaling molecule from Table 1 or a gene encoding thereof). The therapeutic efficacy of the combination treatment can be comparable to a therapeutic efficacy achieved by using the inhibitor against either the first target or the second target, when used alone at a greater amount than that found in the combination treatment. The therapeutic efficacy (e.g., a degree of in vitro growth inhibition of the cancer cells) by the combination treatment can be comparable to that by the inhibitor against the first target alone in an amount greater than that used in the combination by at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100- fold, or more. The therapeutic efficacy (e.g., a degree of in vitro growth inhibition of the cancer cells) by the combination treatment can be comparable to that by the inhibitor against the second target alone in an amount greater than that used in the combination by at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, or more. The practice of any of the methods disclosed herein may utilize a Kras G12D inhibitor disclosed herein (including those disclosed in the Composition section below) in combination or in conjunction of any inhibitor against a signaling molecule exemplified herein (including those shown in Table 1). In some embodiments, a Kras G12D inhibitor utilized in practicing a subject method has a formula selected from any of the formulae of CA to CE and CF′ to CJ′. In some embodiments, Kras G12D inhibitors such as cpds A, B and C having a formula selected from formula CJ′, CA, and CF′ are utilized in the exemplified combinations, and yield enhanced cancer cell growth inhibition when used in combination with an inhibitor against SOS, SHP2, EGFR, MEK, CDK4/6, or PI3Ka. In some embodiment, a subject SOS inhibitor utilized in practicing any of the methods has a formula selected from formulae BB, BC, BC′, CF, CG, CH, CI, CJ, CK, and CK′. As is shown in the exemplified combinations, SOS inhibitor having a formula of CH when used in combination or conjunction with Kras G12D inhibitors (e.g., cpds A, B, and C) yielded robust synergy against multiple types of cancers including pancreatic, colorectal, and gastric cancers, in accordance with the BLISS independent criterion assessment. In some embodiment, a subject SHP2 inhibitor utilized in practicing any of the methods has a formula selected from formulae CL to CZ and DA to DZ. As is shown in the exemplified combinations, SHP2 inhibitor having a formula of CL when used in combination or conjunction with Kras G12D inhibitors (e.g., cpds A, B, and C) yielded robust synergy against multiple types of cancers including pancreatic, colorectal, and gastric cancers, in accordance with the BLISS independent criterion assessment. Similarly, EGFR inhibitors used in combination or conjunction with these Kras G12D inhibitors yielded robust synergy against multiple types of cancers including pancreatic, colorectal, and gastric cancers, in accordance with the BLISS independent criterion assessment. However, only mild to robust synergy was observed using the combination of these exemplified Kras G12D inhibitors with MEK inhibitor, CDK4/6 inhibitor, or PI3Ka inhibitor, in some but not all cancer cell lines tested. It is unexpected that while PI3K-, MEK-, and CDK4/6-pathway “cross-talk” with the Ras pathway, having signaling molecules upstream or downstream to the cross-talked Ras pathway, the combinations of Kras G12D inhibitor with the inhibitor of PI3Ka, MEK, and CDK4/6, respectively, do not yield robust synergy across the cancer cell lines tested in accordance with the BLISS independent criterion assessment.

In some embodiments, a Kras G12D inhibitor utilized in practicing a subject method or included in a subject composition exhibit therapeutically sub-optimal characteristics (including sub-optimal pharmacokinetics (PK) parameters) when used alone, but when combined with a subject second agent, yields an overall increase in efficacy (e.g., promoting one or more desired therapeutic outcomes and/or reducing undesirable side-effects). For example, the exemplified compounds described above exhibit one or more sub-optimal pharmacokinetics (PK) parameters such as poor membrane permeability (e.g., less than about −6 in PAMPA), low bioavailability, short half-life, rapid metabolism, rapid clearance, and/or low area under the curve (AUC). However, when used with one or more additional agents exemplified herein including, e.g., SOS inhibitor, SHP2 inhibitor, EGFR inhibitor, the resulting combination treatments boosted the response and lowered the concentration necessary for the Kras12D inhibitor to achieve 50% growth inhibition (IC50) across the majority of the cancer cell lines tested. The combinations lowered the IC50 for these exemplified Kras12D inhibitors from about 3 to about 10 folds (e.g., against lung cancer (A427 cell line), pancreatic cancer (ASPC1 cell line), colorectal cancer (Ls513 cell line), gastric cancer (AGS cell line).

In some embodiments, an inhibitor of the present disclosure is capable of inhibiting one or more of the following signaling molecules: (1) SOS1 or a mutant thereof (e.g., BAY-293, BI-1701963); (2) SHP2 or a mutant thereof (e.g., 6-(4-amino-4-methylpiperidin-1-yl)-3-(2,3-dichlorophenyl)pyrazin-2-amine, TNO155, RMC-4630, ERAS-601, JAB-3068, IACS-13909/BBP-398, SHP099, RMC-4550); (3) SHC or a mutant thereof (e.g., PP2, AID371185); (4) GAB or a mutant thereof (e.g., GAB-0001); (5) GRB or a mutant thereof; (6) JAK or a mutant thereof (e.g., tofacitinib); (7) A-RAF, B-RAF, C-RAF, or a mutant thereof (e.g., RAF-709, LY-3009120); (8) BRAF or a mutant thereof (e.g., Sorafenib, Vemurafenib, Dabrafenib, Encorafenib, regorafenib, GDC-879); (9) MEK or a mutant thereof (e.g., trametinib, cobimetinib, binimetinib, selumetinib, refametinib, AZD6244); (10) ERK or a mutant thereof (e.g., ulixertinib, MK-8353, LTT462, AZD0364, SCH772984, BIX02189, LY3214996, ravoxertinib); (11) PI3K or a mutant thereof (e.g., Idelalisib, Copanlisib, Duvelisib, Alpelisib, Taselisib, Perifosine, Buparlisib, Umbralisib, NVP-BEZ235-AN); (12) MAPK or a mutant thereof (e.g., VX-745, VX-702, RO-4402257, SCIO-469, BIRB-796, SD-0006, PH-797804, AMG-548, LY2228820, SB-681323, GW-856553, RWJ67657, BCT-197); (13) EGFR or a mutant thereof (e.g., afatinib, erlotinib, gefitinib, lapatinib, cetuximab panitumumab, osimertinib, olmutinib, EGF-816); (14) c-MET or a mutant thereof (e.g., K252a, SU11274, PHA665752, PF2341066); (15) ALK or a mutant thereof (e.g. crizotinib, alectinib, entrectinib, brigatinib); (16) FGFRI, FGFR-2, FGFR-3, FGFR-4 or a mutant thereof (e.g., nintedanib); (17) BCR-ABL or a mutant thereof (e.g., imatinib, dasatinib, nilotinib); (18) ErbB2 (Her2) or a mutant thereof (e.g., afatinib, lapatinib, trastuzumab, pertuzumab); (19) AXL or a mutant thereof (e.g., R428, amuvatinib, XL-880); (20) NTRK1 or a mutant thereof (e.g., merestinib); (21) ROS1 or a mutant thereof (e.g., entrectinib); (22) RET or a mutant thereof (e.g., BLU-667, Lenvatinib); (23) MDM2 or a mutant thereof (e.g., HDM-201, NVP-CGM097, RG-71 12, MK-8242, RG-7388, SAR405838, AMG-232, DS-3032, RG-7775, APG-115); (24) mTOR or a mutant thereof (e.g., rapamycin, temsirolimus, everolimus, ridaforolimus); (25) BET or a mutant thereof (e.g., I-BET 151, I-BET 762, OTX-015, TEN-010, CPI-203, CPI-0610, olionon, RVX-208, ABBC-744, LY294002, AZD5153, MT-1, MS645); (26) IGF1, IGF2, IGFIR, or a mutant thereof (e.g., xentuzumab, MEDI-573); (27) CDK9 or a mutant thereof (e.g., DRB, flavopiridol, CR8, AZD 5438, purvalanol B, AT7519, dinaciclib, SNS-032); or (28) CDK4/6 (e.g., palbociclib, ribociclib, abemaciclib).

In some embodiments, the method disclosed herein further comprises contacting the cell with one or more pharmacologically active substances. The one or more pharmacologically active substances can comprise one or more members from Table 2.

TABLE 2
Exemplary pharmacologically active substances

| No. | Name |
|---|---|
| 1 | an immunotherapeutic agent |
| 2 | a taxane |
| 3 | an antimetabolite |
| 4 | a mitotic kinase inhibitor |
| 5 | an anti-angiogenic drug |
| 6 | a topoisomerase inhibitor |
| 7 | a platinum-containing compound |

In some embodiments, expression or activity of a signal molecule (e.g., (a) Kras G12D and (b) one or more signaling molecules selected from Table 1) is ascertained by a method selected from the group consisting of nucleic acid sequencing, in situ hybridization, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR). In some embodiments, a downregulated expression in a tissue (e.g., a tumor tissue) can be evidenced by a decrease as compared to a control: (a) in a level of mRNA encoding the signal molecule; (b) in a level of cDNA produced from reverse transcription of such mRNA; (c) in a level of functional signal molecule; and/or (d) in a level of cell-free DNA indicative of expression of the signal molecule.

In some embodiments, expression or activity of a signal molecule (e.g., (a) Kras G12D and (b) one or more signaling molecules selected from Table 1) in a cell is permanently downregulated. In some embodiments, expression or activity of the signal molecule is transiently downregulated as compared to a control cell. In some cases, the expression or activity of the signal molecule can be transiently downregulated for at most about 6 months, 5 months, 4 months, 3 months, 2 months, 1 month, 21 days, 14 days, 13 days, 12 days, 11 days, 10 days, 9 days, 8 days, 7 days, 6 days, 5 days, 4 days, 3 days, 48 hours, 44 hours, 40 hours, 36 hours, 32 hours, 28 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 55 minutes, 50 minutes, 45 minutes, 40 minutes, 35 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, or a shorter period of time. In some cases, subsequent to the transient downregulation, at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of the downregulated expression or activity level of the signal molecule can be regained.

In some embodiments, therapeutically effective plasma concentration of any inhibitor disclosed herein is at least about 1 nanomolar (nM), 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 micromolar (μM), 2 μM, 3 μM, 4 μM, 5 μM, 6 μM, 7 μM, 8 μM, 9 μM, 10 μM, or more for a duration of time. In some cases, a therapeutically effective plasma concentration of any inhibitor disclosed herein is at most about 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 21 nM, or less for a duration of time. Such duration of time may be at least about 0.1 hour, 0.2 hour, 0.3 hour, 0.4 hour, 0.5 hour, 0.6 hour, 0.7 hour, 0.8 hour, 0.9 hour, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer.

In some embodiments, a cell can be contacted with any of the inhibitor disclosed herein and the one or more pharmacologically active substances either simultaneously or sequentially (e.g., contacting the cell with any of the inhibitor before or after contacting the cell with the one or more pharmacologically active substances). For simultaneous contacting, any of the inhibitor and the one or more pharmacologically active substances can be in the same composition (or formulation) or in different compositions (e.g., subjecting the cell to two different compositions at the same time). In some embodiments, a subject comprising the cell is administered with any of the inhibitor and the one or more pharmacologically active substances. The subject can be administered with any of the inhibitor and the one or more pharmacologically active substances either simultaneously or sequentially (e.g., administration of any of the inhibitor before or after administration of the one or more pharmacologically active substances). For simultaneous administration, any of the inhibitor and the one or more pharmacologically active substances can be in the same composition (or formulation) or in different compositions (e.g., administration of two different compositions at the same time to the same location or to different locations of the subject's body). For sequential administration, any of the inhibitor and the one or more pharmacologically active substances can be administered in different compositions. For sequential administration, a first administration (e.g., administration of one of any of the inhibitor and the one or more pharmacologically active substances) and a second administration (e.g., administration of the other of any of the inhibitor and the one or more pharmacologically active substances) can be separated by at least about 1, 2, 3, 4, 5, 6, 7, 8, 9, 10, 20, 30, 40, 50, 60 minutes, 2, 3, 4, 5, 6, 7, 8, 9, 10, 11, 12, 16, 20, 24 hours, or more. The first administration and the second administration can be separated by at most about 24, 20, 16, 12, 11, 10, 9, 8, 7, 6, 5, 4, 3, 2 hours, 60, 50, 40, 30, 20, 10, 9, 8, 7, 6, 5, 4, 3, 2, 1 minute, or less.

In any of the methods disclosed herein, phosphorylation of a substrate or a specific amino acid residue thereof can be detected and/or quantified one or more techniques, such as kinase activity assays, phospho-specific antibodies, Western blot, enzyme-linked immunosorbent assays (ELISA), cell-based ELISA, intracellular flow cytometry, mass spectrometry, and multi-analyte profiling.

The cell disclosed herein can be a diseased cell. The diseased cell may be derived from or disposed in excretions or body tissues of a subject, e.g., skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, etc.

In some embodiments, the diseased cell is a cancer cell from a wide variety of cancers, including both solid tumor and hematological cancers, such as, Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma—see Non-Hodgkin Lymphoma, Carcinoid Tumor (Gastrointestinal), Childhood Carcinoid Tumors, Cardiac (Heart) Tumors, Atypical Teratoid/Rhabdoid Tumor, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sézary Syndrome), Ductal Carcinoma In Situ (DCIS), Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Hodgkin Lymphoma, Hypopharyngeal Cancer (Head and Neck Cancer), Islet Cell Tumors, Pancreatic Neuroendocrine Tumors, Kaposi Sarcoma (Soft Tissue Sarcoma), Kidney (Renal Cell) Cancer, Laryngeal Cancer (Head and Neck Cancer), Leukemia, Lip and Oral Cavity Cancer (Head and Neck Cancer), Liver Cancer, Lung Cancer (e.g., Non-Small Cell and Small Cell), Lymphoma, Male Breast Cancer, Malignant Fibrous Histiocytoma of Bone and Osteosarcoma, Melanoma, Merkel Cell Carcinoma (Skin Cancer), Mesothelioma, Malignant, Metastatic Cancer, Metastatic Squamous Neck Cancer with Occult Primary (Head and Neck Cancer), Midline Tract Carcinoma, Mouth Cancer (Head and Neck Cancer), Multiple Endocrine Neoplasia, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, CML, Myeloid Leukemia, Acute (AML), Myeloproliferative Neoplasms, Chronic, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Rectal Cancer, Retinoblastoma, Rhabdomyosarcoma, Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma(Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors, and any of the aforementioned cancers exhibiting an aberrantly high cell proliferation activity.

In some cases, tumor cells, as described herein, can be derived from one or more tumor cell lines. A tumor cell line can be derived from a tumor in one or more tissues, e.g., pancreas, lung, ovary, biliary tract, intestine (e.g., small intestine, large intestine (i.e. colon)), endometrium, stomach, hematopoietic tissue (e.g., lymphoid tissue), etc. Examples of the tumor cell line with a K-Ras mutation may include, but are not limited to, A549 (e.g., K-Ras G12S), AGS (e.g., K-Ras G12D), ASPC1 (e.g., K-Ras G12D), Calu-6 (e.g., K-Ras Q61K), CFPAC-1 (e.g., K-Ras G12V), CL40 (e.g., K-Ras G12D), COL0678 (e.g., K-Ras G12D), COR-L23 (e.g., K-Ras G12V), DAN-G (e.g., K-Ras G12V), GP2D (e.g., K-Ras G12D), GSU (e.g., K-Ras G12F), HCT116 (e.g., K-Ras G13D), HEC1A (e.g., K-Ras G12D), HEC1B (e.g., K-Ras G12F), HEC50B (e.g., K-Ras G12F), HEYA8 (e.g., K-Ras G12D or G13D), HPAC (e.g., K-Ras G12D), HPAFII (e.g., K-Ras G12D), HUCCT1 (e.g., K-Ras G12D), KARPAS620 (e.g., K-Ras G13D), KOPN8 (e.g., K-Ras G13D), KP-3 (e.g., K-Ras G12V), KP-4 (e.g., K-Ras G12D), L3.3 (e.g., K-Ras G12D), LoVo (e.g., K-Ras G13D), LS180 (e.g., K-Ras G12D), LS513 (e.g., K-Ras G12D), MCAS (e.g., K-Ras G12D), NB4 (e.g., K-Ras A18D), NCI-H1355 (e.g., K-Ras G13C), NCI-H1573 (e.g., K-Ras G12A), NCI-H1944 (e.g., K-Ras G13D), NCI-H2009 (e.g., K-Ras G12A), NCI-H441 (e.g., K-Ras G12V), NCI-H747 (e.g., K-Ras G13D), NOMO-1 (e.g., K-Ras G12D), OV7 (e.g., K-Ras G12D), PANC0203 (e.g., K-Ras G12D), PANC0403 (e.g., K-Ras G12D), PANC0504 (e.g., K-Ras G12D), PANC0813 (e.g., K-Ras G12D), PANC1 (e.g., K-Ras G12D), Panc-10.05 (e.g., K-Ras G12D), PaTu-8902 (e.g., K-Ras G12V), PK1 (e.g., K-Ras G12D), PK45H (e.g., K-Ras G12D), PK59 (e.g., K-Ras G12D), SK-CO-1 (e.g., K-Ras G12V), SKLU1 (e.g., K-Ras G12D), SKM-1 (e.g., K-Ras K117N), SNU1 (e.g., K-Ras G12D), SNU1033 (e.g., K-Ras G12D), SNU1197 (e.g., K-Ras G12D), SNU407 (e.g., K-Ras G12D), SNU410 (e.g., K-Ras G12D), SNU601 (e.g., K-Ras G12D), SNU61 (e.g., K-Ras G12D), SNU8 (e.g., K-Ras G12D), SNU869 (e.g., K-Ras G12D), SNU-C2A (e.g., K-Ras G12D), SU.86.86 (e.g., K-Ras G12D), SUIT2 (e.g., K-Ras G12D), SW1990 (e.g., K-Ras G12D), SW403 (e.g., K-Ras G12V), SW480 (e.g., K-Ras G12V), SW620 (e.g., K-Ras G12V), SW948 (e.g., K-Ras Q61L), T3M10 (e.g., K-Ras G12D), TCC-PAN2 (e.g., K-Ras G12R), TGBC11TKB (e.g., K-Ras G12D), and MIA Pa-Ca (e.g., MIA Pa-Ca 2 (e.g., K-Ras G12C)).

Another aspect of the present disclosure provided a cell that is modified (i.e., a modified cell) by any of the methods disclosed herein. In some embodiments, a modified cell is characterized by exhibiting inhibition of cell signaling, such as cell proliferation signaling. In some embodiments, a modified cell is characterized by exhibiting downregulated expression and/or activity of (a) a Ras protein (e.g., a mutated Ras protein such as Kras G12D) and that of (b) one or more signaling molecules selected from Table 1. In some examples, the modified cell may have been treated with any of the inhibitors disclosed herein, e.g., an inhibitor against (a) a Ras protein (e.g., a mutant Ras, such as Kras G12D) and/or an inhibitor against (b) one or more signaling molecules selected from Table 1. In some embodiments, the modified cell exhibits reduced Ras signaling output in in the modified cell, as provided herein. In some examples, the modified cell has been treated with any of the pharmacologically active substances selected from Table 2.

In some embodiments, the modified cell comprises any of the inhibitors disclosed herein, e.g., an inhibitor against (a) a Ras protein (e.g., a mutant Ras, such as Kras G12D) and/or an inhibitor against (b) one or more signaling molecules selected from Table 1.

In some examples, the cell uptakes a subject inhibitor via endocytosis. In some examples, a subject inhibitor is passed across the plasma membrane of the cell via transfection (e.g., via transfection agents, such as polymers, nanoparticles, etc.), electroporation, microinjection, or infection (e.g., via viral or non-viral delivery carriers). In some examples, a subject inhibitor is expressed (e.g., synthesized) by the cell upon contacting the cell by contacting the cell with a gene encoding the subject inhibitor. Such gene encoding the subject inhibitor can be heterologous to the cell. The gene can be introduced to the cell via transfection, electroporation, microinjection, or infection, as provided herein. The gene can be integrated into a genome of the cell. Alternatively, the gene may not or need not be integrated into the genome of the cell. In some embodiments, the modified cell comprises any of the pharmacologically active substances selected from Table 2.

Examples of a viral delivery vehicle comprises an adenovirus, a retrovirus, a lentivirus (e.g., a human immunodeficiency virus (HIV)), an adeno-associated virus (AAV), and/or a Herpes simplex virus (HSV). In an example, the viral delivery vehicle may be a retrovirus. The retrovirus may be a gamma-retrovirus selected from the group consisting of: Feline Leukemia Virus (FLV), Feline Sarcoma Virus (Strain Hardy-Zuckerman 4), Finkel-Biskis-Jinkins Murine Sarcoma Virus (FBJMSV), Murine leukemia virus (MLV) (e.g. Friend Murine Leukemia Virus (FMLV), Moloney Murine Leukemia Virus (MMLV), Murine Type C Retrovirus (MTCR)), Gibbon Ape Leukemia Virus (GALV), Koala Retrovirus (KR), Moloney Murine Sarcoma Virus (MMSV), Porcine Endogenous Retrovirus E (PERE), Reticuloendotheliosis Virus (RV), Woolly Monkey Sarcoma Virus (WMSV), Baboon Endogenous Virus Strain M7 (BEVSM7), Murine Osteosarcoma Virus (MOV), Mus Musculus Mobilized Endogenous Polytropic Provirus (MMMEPP), PreXMRV-1, RD114 Retrovirus, Spleen Focus-Forming Virus (SFFV), Abelson murine leukemia virus (AMLV), Murine Stem Cell Virus (MSCV), and variants thereof.

Examples of a non-viral delivery vehicle comprises nanoparticles, nanospheres, nanocapsules, microparticles, microspheres, microcapsules, liposomes, nanoemulsions, solid lipid nanoparticles, modifications thereof, or combinations thereof. The non-viral delivery vehicle of the present invention may be prepared by methods, such as, but not limited to, nanoprecipitation, emulsion solvent evaporation method, emulsion-crosslinking method, emulsion solvent diffusion method, microemulsion method, gas antisolvent precipitation method, ionic gelation methods milling or size reduction method, PEGylation method, salting-out method, dialysis method, single or double emulsification method, nanospray drying method, layer by layer method, desolvation method, supercritical fluid technology, supramolecular assembly, or combinations thereof.

The subject disclosed herein can be a human subject. Certain other embodiments contemplate a non-human subject, for example a non-human primate such as a macaque, chimpanzee, gorilla, vervet, orangutan, baboon or other non-human primate, including such non-human subjects that can be known to the art as preclinical models, the tumor tissue or cancer cells of which exhibit, for example, aberrantly high expression of cell proliferation signaling. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source.

The methods disclosed herein can be applied to treat and/or ameliorate symptoms of a wide variety of cancers in a subject, including both solid tumor and hematological cancers. For example, a subject in need of a treatment may suffer from a hematological cancer, a solid cancer, or a combination thereof. The cancer can be a hematologic cancer, e.g., a cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. The cancer can also be chosen from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, a subject suffers from one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL). In some embodiments, the lymphoma is mantle cell lymphoma (MCL), T cell lymphoma, Hodgkin's lymphoma, and/or non-Hodgkin's lymphoma, nephroblastoma, Ewing's sarcoma, neuroendocrine tumor, glioblastoma, neuroblastoma, melanoma, skin cancer, breast cancer, colon cancer, rectal cancer, prostate cancer, liver cancer, kidney cancer, pancreatic cancer, lung cancer, biliary tract cancer, cervical cancer, endometrial cancer, esophageal cancer, gastric cancer, head and neck cancer, medullary thyroid carcinoma, ovarian cancer, glioma, and bladder cancer.

In some embodiments, inhibition of cell proliferation signaling in a cell (e.g., a cancer cell) in a subject evidenced by tumor being stabilized (e.g., one or more tumors do not increase more than 1%, 5%, 10%, 15%, or 20% in size, and/or do not metastasize) as a result of treatment with a subject method disclosed herein. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95%, or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

Where desired, a subject (e.g., a human subject) can be screened for the presence of a Kras mutation, such as Kras G12D mutation. The subject can also be screened for the retention of expression and/or activity of a mutated Kras protein (e.g., Kras G12D) in one or more types of subject's cells (e.g., cancer cells).

In some embodiments, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof.

In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/M4X-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), and olaparib (LYNPARZA®).

Anti-cancer agents of particular interest for combinations with any inhibitor disclosed herein include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, RevimmuneT), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In an aspect, compositions provided herein can be administered in combination with radiotherapy such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-11BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody.

In some embodiments, the checkpoint immune blockade agents are selected from the group consisting of anti-PD-LI antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7W, 410C9), anti-B7-H3 antibodies (e.g., DS-5573a), anti-TIM3 antibodies (e.g., F38-2E2), and combinations thereof. In one embodiment, the checkpoint immune blockade agent is an anti-PD-LI antibody. In some cases, any inhibitor disclosed herein can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising any inhibitor disclosed herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldeskeukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg.

In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In combination treatment, any inhibitor disclosed herein and other anti-cancer agent(s) may be administered either simultaneously, concurrently or sequentially with no specific time limits, wherein such administration provides therapeutically effective levels of the two compounds in the body of the patient.

In a preferred embodiment, any inhibitor disclosed herein and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

An antibiotic can be administered to a subject as part of a therapeutic regime. An antibiotic can be administered at a therapeutically effective dose. An antibiotic can kill or inhibit growth of bacteria. An antibiotic can be a broad spectrum antibiotic that can target a wide range of bacteria. Broad spectrum antibiotics, either a 3¹ or 4¹ generation, can be cephalosporin or a quinolone.

An antibiotic can also be a narrow spectrum antibiotic that can target specific types of bacteria. An antibiotic can target a bacterial cell wall such as penicillins and cephalosporins. An antibiotic can target a cellular membrane such as polymyxins. An antibiotic can interfere with essential bacterial enzymes such as antibiotics: rifamycins, lipiarmycins, quinolones, and sulfonamides. An antibiotic can also be a protein synthesis inhibitor such as macrolides, lincosamides, and tetracyclines. An antibiotic can also be a cyclic lipopeptide such as daptomycin, glycylcyclines such as tigecycline, oxazolidiones such as linezolid, and lipiarmycins such as fidaxomicin. In some cases, an antibiotic can be 1^{st} generation, 2^{nd} generation, 3^{rd} generation, 4th generation, or 5^{th} generation. A first-generation antibiotic can have a narrow spectrum. Examples of 1^{st} generation antibiotics can be penicillins (Penicillin G or Penicillin V), Cephalosporins (Cephazolin, Cephalothin, Cephapirin, Cephalethin, Cephradin, or Cephadroxin). In some cases, an antibiotic can be 2^{nd} generation. 2^{nd} generation antibiotics can be a penicillin (Amoxicillin or Ampicillin), Cephalosporin (Cefuroxime, Cephamandole, Cephoxitin, Cephaclor, Cephrozil, Loracarbef). In some cases, an antibiotic can be 3^{rd} generation. A 3^{rd} generation antibiotic can be penicillin (carbenicillin and ticarcillin) or cephalosporin (Cephixime, Cephtriaxone, Cephotaxime, Cephtizoxime, and Cephtazidime). An antibiotic can also be a 4^{th} generation antibiotic. A 4^{th} generation antibiotic can be Cephipime. An antibiotic can also be 5^{th} generation. 5^{th} generation antibiotics can be Cephtaroline or Cephtobiprole.

In some cases, an anti-viral agent may be administered as part of a treatment regime. In some cases, a herpes virus prophylaxis can be administered to a subject as part of a treatment regime. A herpes virus prophylaxis can be valacyclovir (Valtrex). Valtrex can be used orally to prevent the occurrence of herpes virus infections in subjects with positive HSV serology. It can be supplied in 500 mg tablets. Valacyclovir can be administered at a therapeutically effective amount.

In some cases, a treatment regime may be dosed according to a body weight of a subject. In subjects who are determined obese (BMI>35) a practical weight may need to be utilized. BMI is calculated by: BMI=weight (kg)/[height (m)]².

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight.

An adjusted body weight may be the sum of an ideal body weight+(0.4×(Actual body weight−ideal body weight)). In some cases a body surface area may be utilized to calculate a dosage. A body surface area (BSA) may be calculated by: BSA (m2)=√Height (cm)*Weight (kg)/3600.

Any subject composition of formulation disclosed herein can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

In embodiment, the subject method comprises administering a Kras G12D inhibitor having a formula selected from formulae CA to CE and CF′ to CJ′ and any embodiments thereof, as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having a formula selected from formulae CA to CE and CF′ to CJ′ and any embodiments thereof, as disclosed herein, in combination or conjunction with an inhibitor of SHP2 selected from

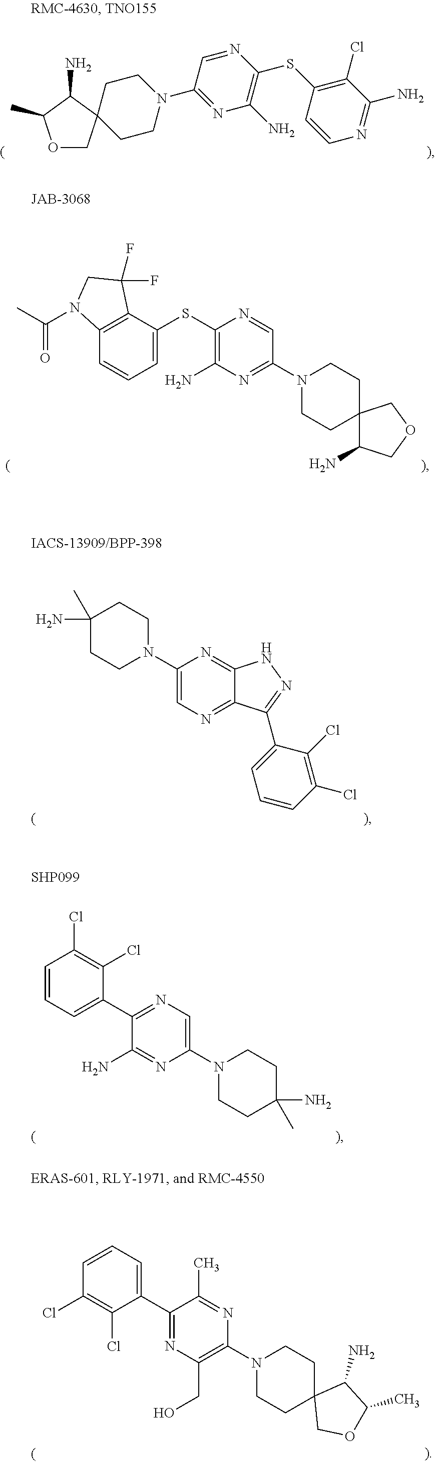

In embodiment, the subject method comprises administering a Kras G12D inhibitor having a formula selected from formulae CA to CE and CF′ to CJ′ and any embodiments thereof, as disclosed herein, in combination or conjunction with an inhibitor of SOS selected from

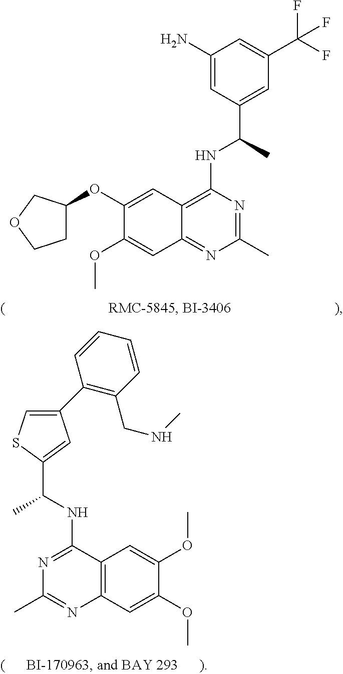

In embodiment, the subject method comprises administering a Kras G12D inhibitor having a formula selected from formulae CA to CE and CF′ to CJ′ and any embodiments thereof, as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having a formula selected from formulae CA to CE and CF′ to CJ′ and any embodiments thereof, as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having a formula selected from formulae CA to CE and CF′ to CJ′ and any embodiments thereof, as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having a formula selected from formulae CA to CE and CF′ to CJ′ and any embodiments thereof, as disclosed herein, in combination or conjunction with an inhibitor of CDK4/6 selected from palbociclib, ribociclib, and abemaciclib. In embodiment, the subject method comprises administering a Kras G12D inhibitor having a formula selected from formulae CA to CE and CF′ to CJ′ and any embodiments thereof, as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CA), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CA), as disclosed herein, in combination of conjunction with an inhibitor of SHP2 selected from

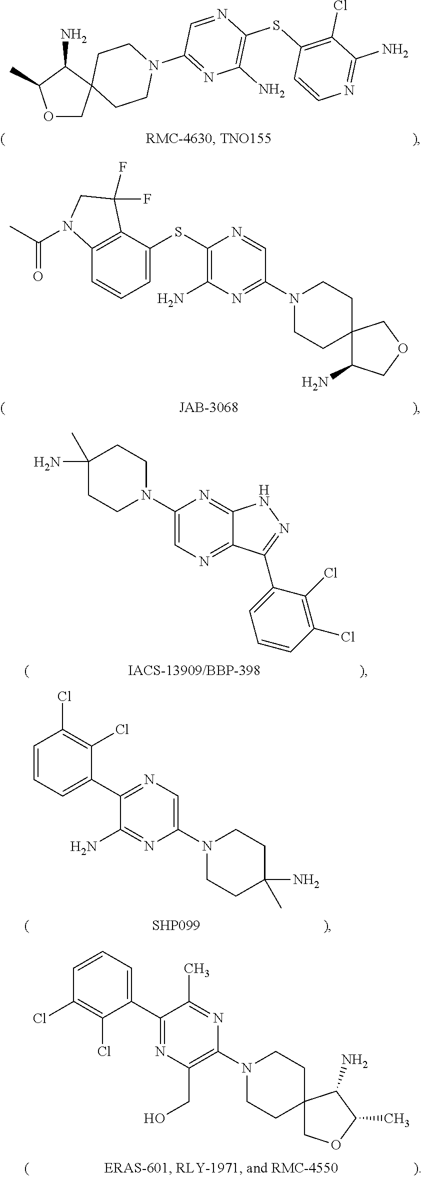 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CA), as disclosed herein, in combination or conjunction with an inhibitor of SOS selected from

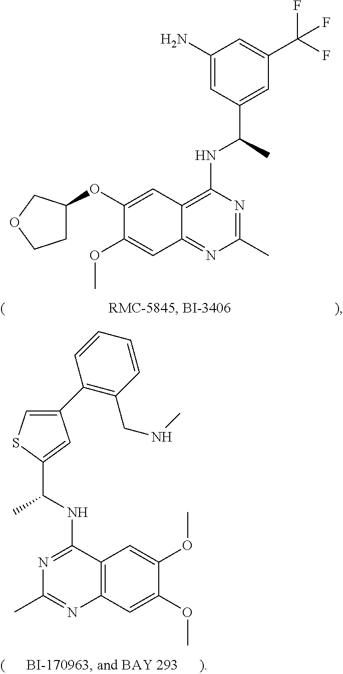

In embodiment, the subject method comprises administering a Kras G12D inhibitor having the formula (CA), as disclosed herein, in combination or conjunction with an inhibitor of EGFR selected from afatinib, erlotinib, gefitinib, lapatinib, cetuximab panitumumab, osimertinib, olmutinib, and EGF-816. In embodiment, the subject method comprises administering a Kras G12D inhibitor having the formula (CA), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CA), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CA), as disclosed herein, in combination or conjunction with an inhibitor of CDK4/6 selected from palbociclib, ribociclib, and abemaciclib. In embodiment, the subject method comprises administering a Kras G12D inhibitor having the formula (CA), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CB), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CB), as disclosed herein, in combination of conjunction with an inhibitor of SHP2 selected from

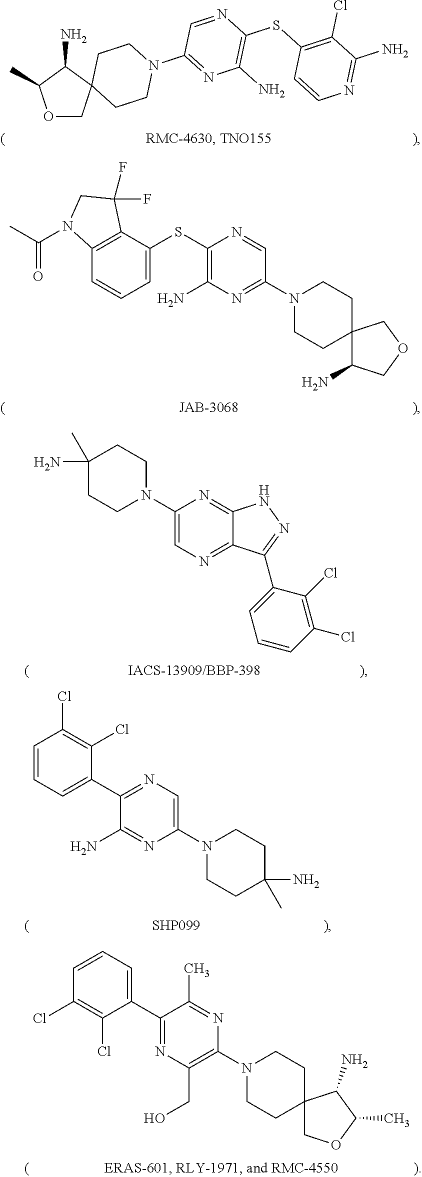 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CB), as disclosed herein, in combination or conjunction with an inhibitor of SOS selected from

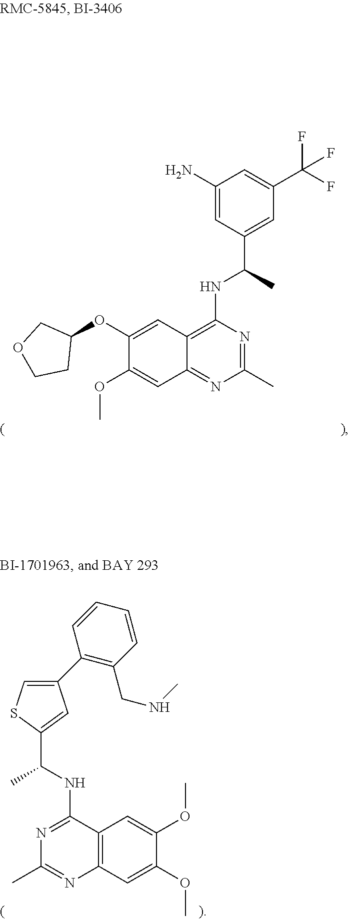

In embodiment, the subject method comprises administering a Kras G12D inhibitor having the formula (CB), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CB), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CB), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CB), as disclosed herein, in combination or conjunction with an inhibitor of CDK4/6 selected from palbociclib, ribociclib, and abemaciclib. In embodiment, the subject method comprises administering a Kras G12D inhibitor having the formula (CB), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CC), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CC), as disclosed herein, in combination of conjunction with an inhibitor of SHP2 selected from

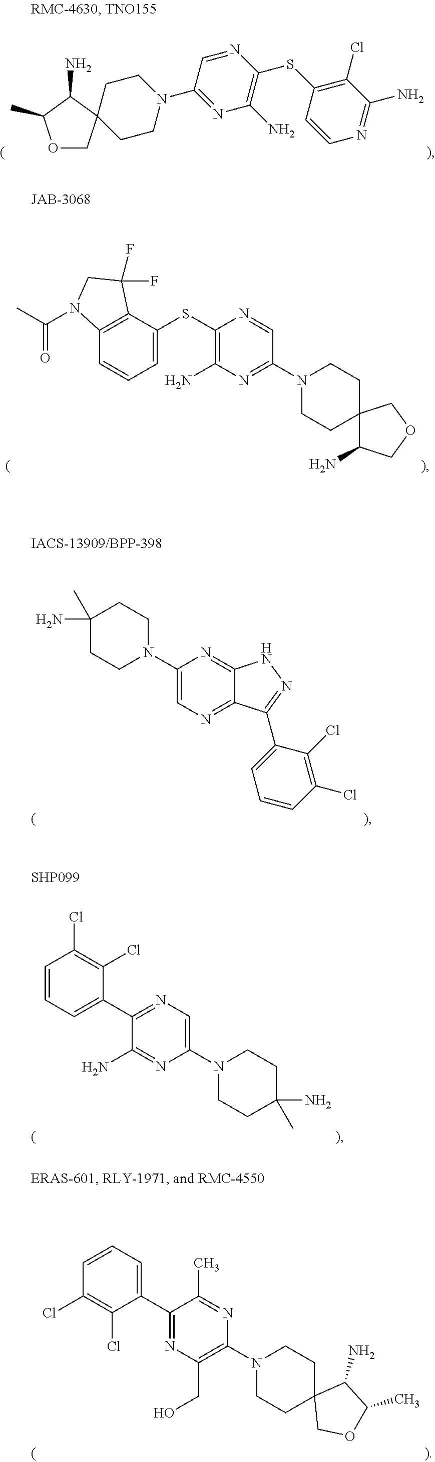

In embodiment, the subject method comprises administering a Kras G12D inhibitor having the formula (CC), as disclosed herein, in combination or conjunction with an inhibitor of SOS selected from

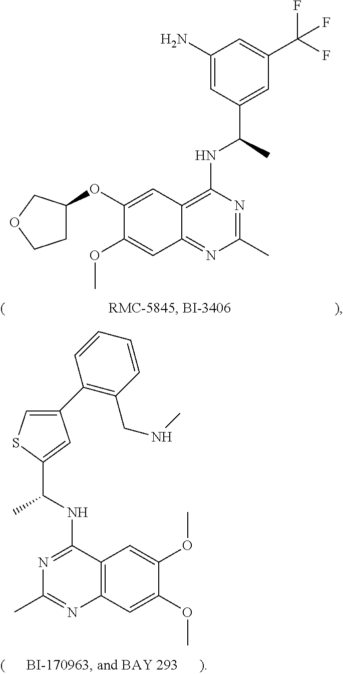

In embodiment, the subject method comprises administering a Kras G12D inhibitor having the formula (CC), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CC), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CC), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CC), as disclosed herein, in combination or conjunction with an inhibitor of CDK4/6 selected from palbociclib, ribociclib, and abemaciclib. In embodiment, the subject method comprises administering a Kras G12D inhibitor having the formula (CC), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CD), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CD), as disclosed herein, in combination of conjunction with an inhibitor of SHP2 selected from

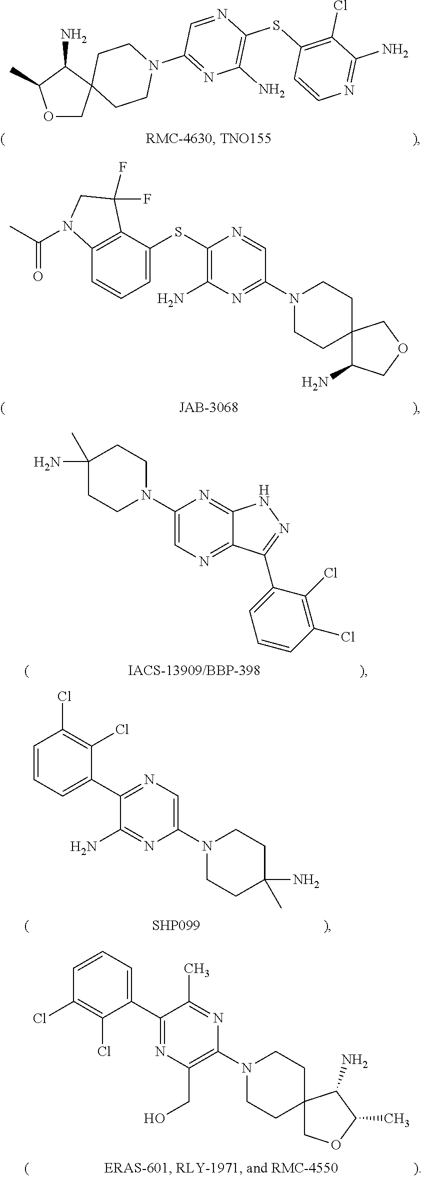 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CD), as disclosed herein, in combination or conjunction with an inhibitor of SOS selected from

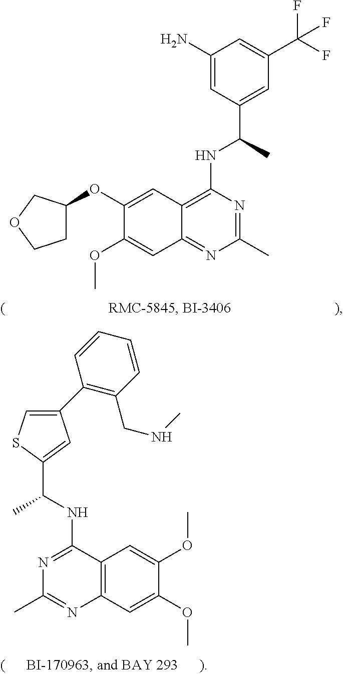

In embodiment, the subject method comprises administering a Kras G12D inhibitor having the formula (CD), as disclosed herein, in combination or conjunction with an inhibitor of EGFR selected from afatinib, erlotinib, gefitinib, lapatinib, cetuximab panitumumab, osimertinib, olmutinib, and EGF-816. In embodiment, the subject method comprises administering a Kras G12D inhibitor having the formula (CD), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CD), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CD), as disclosed herein, in combination or conjunction with an inhibitor of CDK4/6 selected from palbociclib, ribociclib, and abemaciclib. In embodiment, the subject method comprises administering a Kras G12D inhibitor having the formula (CD), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CE), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CE), as disclosed herein, in combination of conjunction with an inhibitor of SHP2 selected from

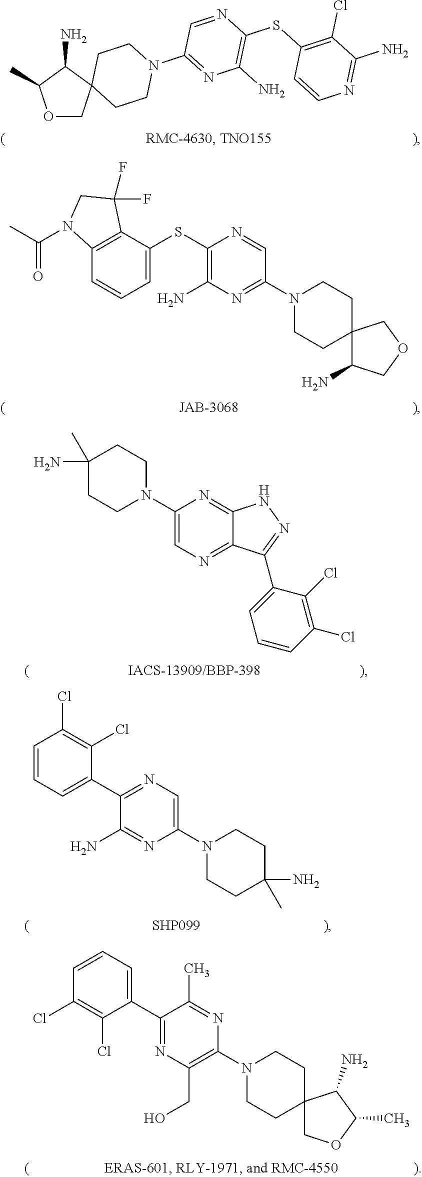 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CE), as disclosed herein, in combination or conjunction with an inhibitor of SOS selected from

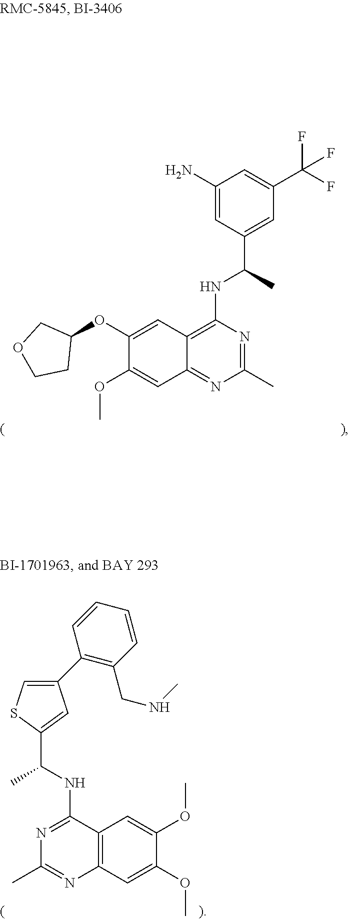

In embodiment, the subject method comprises administering a Kras G12D inhibitor having the formula (CE), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CE), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CE), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CE), as disclosed herein, in combination or conjunction with an inhibitor of CDK4/6 selected from palbociclib, ribociclib, and abemaciclib. In embodiment, the subject method comprises administering a Kras G12D inhibitor having the formula (CE), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CF′), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CF′), as disclosed herein, in combination of conjunction with an inhibitor of SHP2 selected from

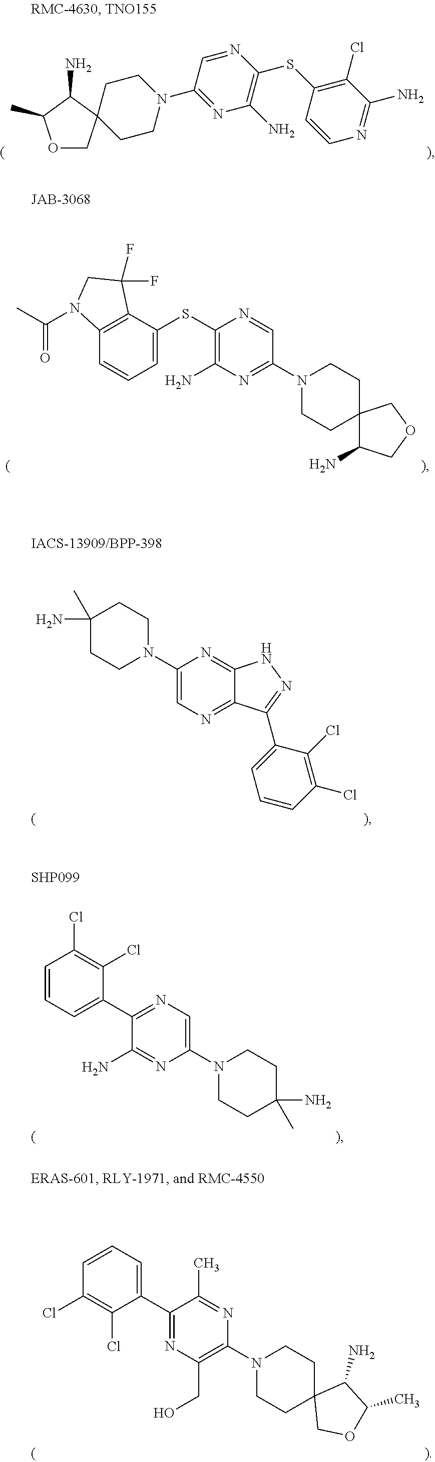

In embodiment, the subject method comprises administering a Kras G12D inhibitor having the formula (CF′), as disclosed herein, in combination or conjunction with an inhibitor of SOS selected from

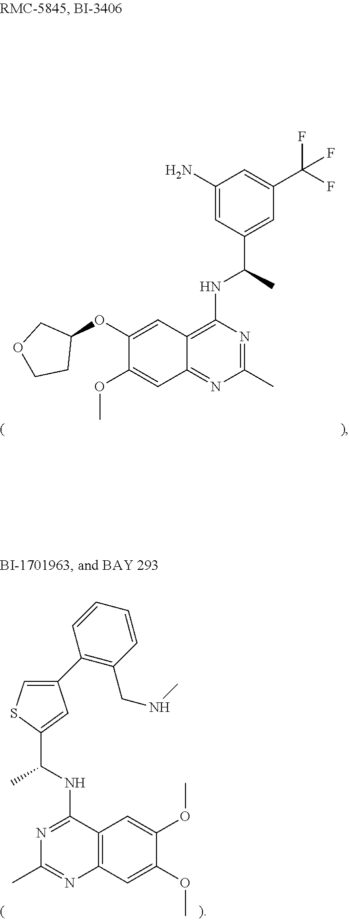

In embodiment, the subject method comprises administering a Kras G12D inhibitor having the formula (CF′), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CF′), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CF′), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CF′), as disclosed herein, in combination or conjunction with an inhibitor of CDK4/6 selected from palbociclib, ribociclib, and abemaciclib. In embodiment, the subject method comprises administering a Kras G12D inhibitor having the formula (CF′), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CG′), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CG′), as disclosed herein, in combination of conjunction with an inhibitor of SHP2 selected from

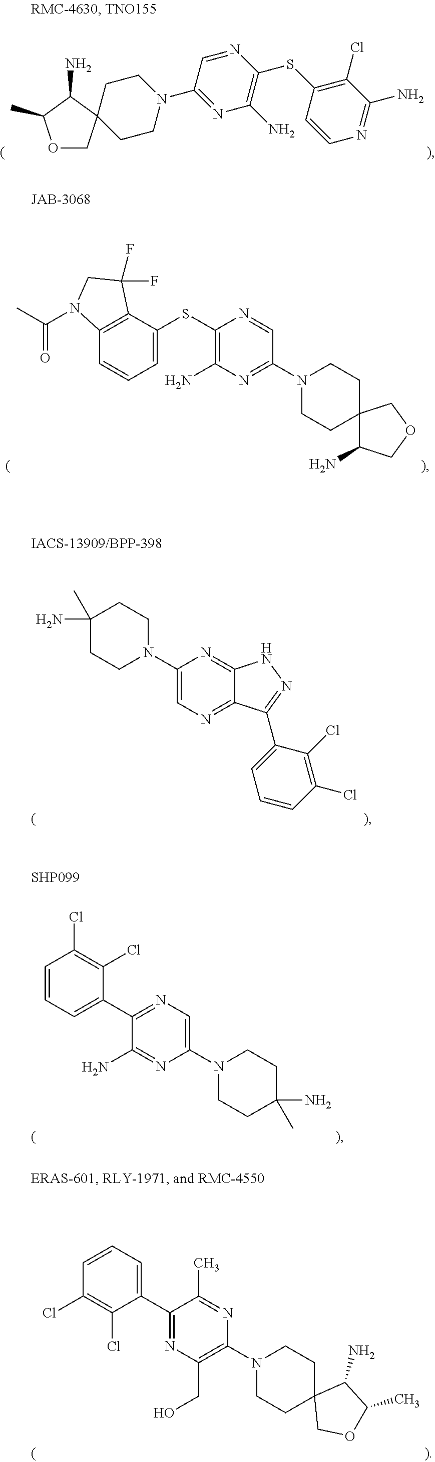

In embodiment, the subject method comprises administering a Kras G12D inhibitor having the formula (CG′), as disclosed herein, in combination or conjunction with an inhibitor of SOS selected from

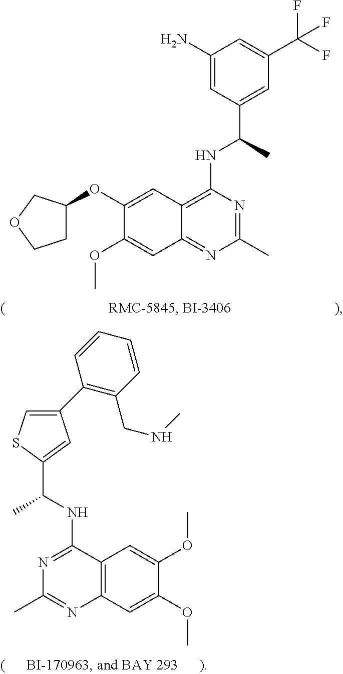

In embodiment, the subject method comprises administering a Kras G12D inhibitor having the formula (CG′), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CG′), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CG′), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CG′), as disclosed herein, in combination or conjunction with an inhibitor of CDK4/6 selected from palbociclib, ribociclib, and abemaciclib. In embodiment, the subject method comprises administering a Kras G12D inhibitor having the formula (CG′), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CH′), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CH′), as disclosed herein, in combination of conjunction with an inhibitor of SHP2 selected from

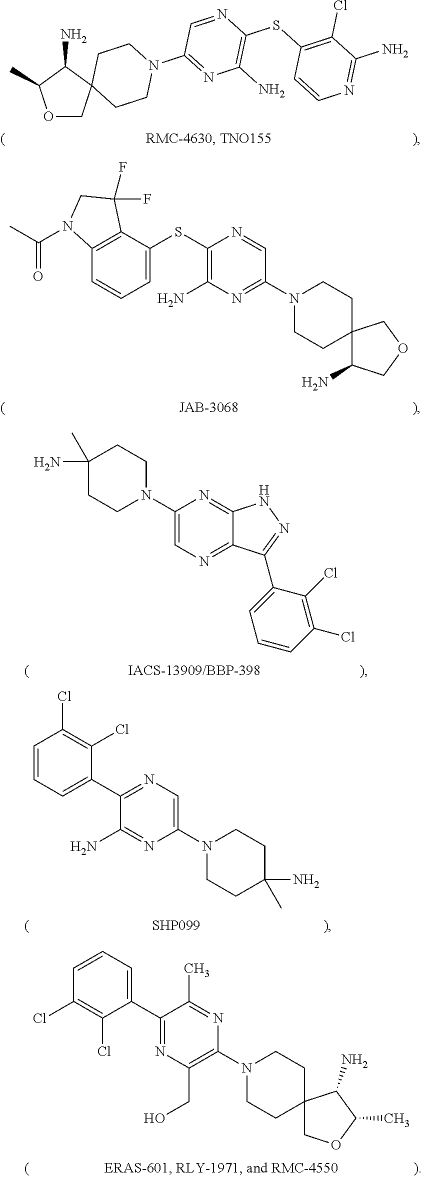 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CH′), as disclosed herein, in combination or conjunction with an inhibitor of SOS selected from

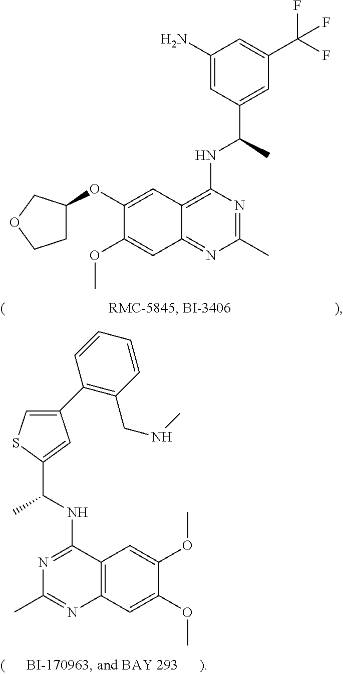

In embodiment, the subject method comprises administering a Kras G12D inhibitor having the formula (CH′), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CH′), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CH′), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CH′), as disclosed herein, in combination or conjunction with an inhibitor of CDK4/6 selected from palbociclib, ribociclib, and abemaciclib. In embodiment, the subject method comprises administering a Kras G12D inhibitor having the formula (CH′), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CI′), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CI′), as disclosed herein, in combination of conjunction with an inhibitor of SHP2 selected from

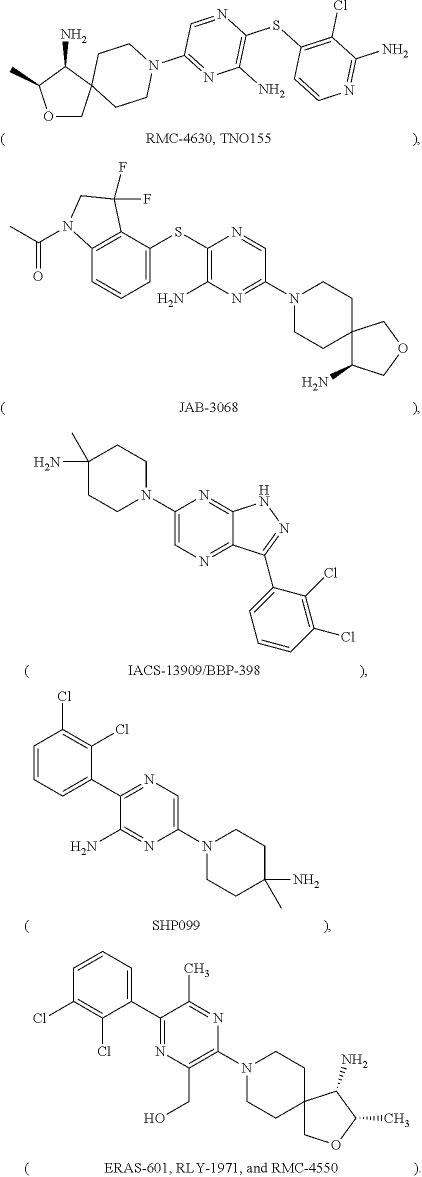 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CI′) as disclosed herein, in combination or conjunction with an inhibitor of SOS selected from

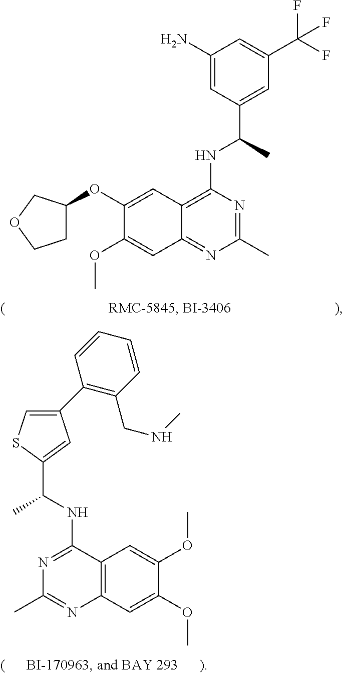

In embodiment, the subject method comprises administering a Kras G12D inhibitor having the formula (CI′), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CI′), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CI′), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CI′), as disclosed herein, in combination or conjunction with an inhibitor of CDK4/6 selected from palbociclib, ribociclib, and abemaciclib. In embodiment, the subject method comprises administering a Kras G12D inhibitor having the formula (CI′), as disclosed herein, in combination or conjunction with an inhibitor of BRAF selected from Sorafenib, Vemurafenib, Dabrafenib, Encorafenib, regorafenib, and GDC-879.

In embodiment, the subject method comprises administering a Kras G12D inhibitor having the formula (CJ′), as disclosed herein, in combination or conjunction with an inhibitor of one or more signaling molecules selected from Table 1 having a formula selected from formulae CF to CZ, CK′, DA to DZ, A to F, N to Z, Z′, AA, AA′, BB, BC, and BC′. In embodiment, the subject method comprises administering a Kras G12D inhibitor having the formula (CJ′), as disclosed herein, in combination of conjunction with an inhibitor of SHP2 selected from

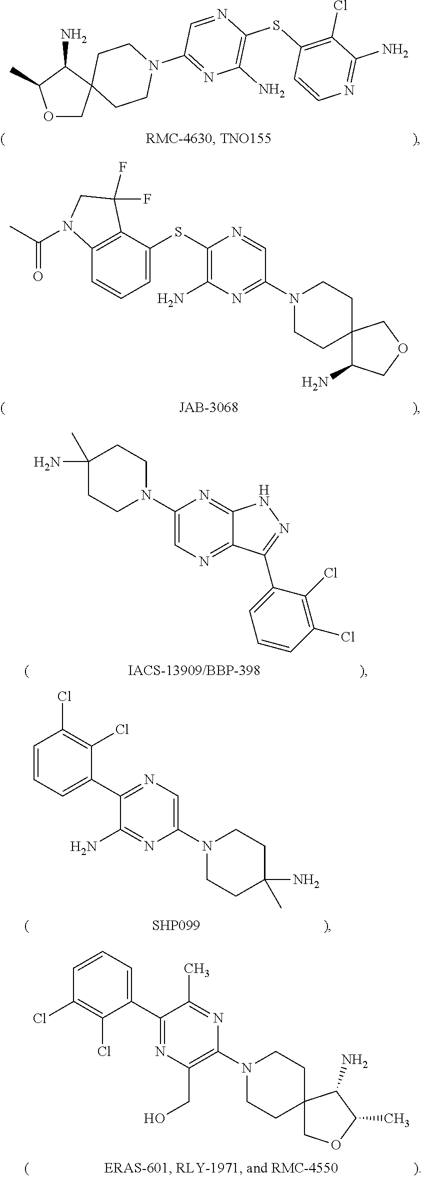 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiment, the subject method comprises administering a Kras G12D inhibitor having the formula (CJ′), as disclosed herein, in combination or conjunction with an inhibitor of SOS selected from

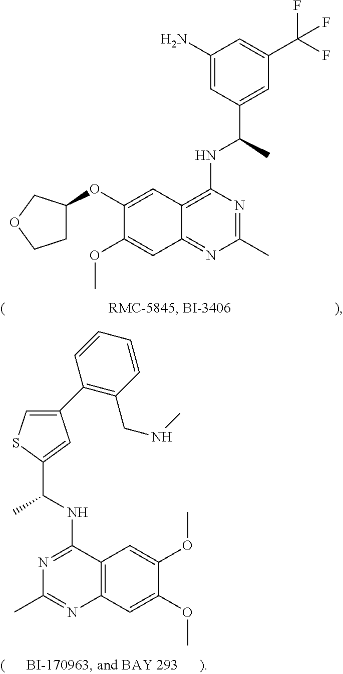

In embodiment, the subject method comprises administering a Kras G12D inhibitor having the formula (CJ), as disclosed herein, in combination or conjunction with an inhibitor of EGFR selected from afatinib, erlotinib, gefitinib, lapatinib, cetuximab panitumumab, osimertinib, olnutinib, and EGF-816. In embodiment, the subject method comprises administering a Kras G12D inhibitor having the formula (CJ′), as disclosed herein, in combination or conjunction with an inhibitor of MEK selected from trametinib, cobimetinib, binimetinib, selumetinib, refametinib, and AZD6244. In embodiment, the subject method comprises administering a Kras G12D inhibitor having the formula (CJ′), as disclosed herein, in combination or conjunction with an inhibitor of ERK selected from ulixertinib, MK-8353, LTT462, AZD0364, SCH772984, BIX02189, LY3214996, and ravoxertinib. In embodiment, the subject method comprises administering a Kras G12D inhibitor having the formula (CJ′), as disclosed herein, in combination or conjunction with an inhibitor of CDK4/6 selected from palbociclib, ribociclib, and abemaciclib. In embodiment, the subject method comprises administering a Kras G12D inhibitor having the formula (CJ′), as disclosed herein, in combination or conjunction with an inhibitor of BRAF selected from Sorafenib, Vemurafenib, Dabrafenib, Encorafenib, regorafenib, and GDC-879.

In embodiments of the subject method, the Kras G12D inhibitor has a formula selected from formulae CF′ to CJ′, as disclosed herein, and wherein the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has a formula selected from formulae CF′ to CJ′, as disclosed herein, and wherein the at least one inhibitor has a formula BB, BC, BC′, CF, CG, CH, CI, CJ, CK, and CK′. In embodiments of the subject method, the Kras G12D inhibitor has a formula selected from formulae CF′ to CJ′, as disclosed herein, and wherein the at least one inhibitor has a formula CL to CZ and DA to DZ. In embodiments of the subject method, the combination comprises (a) a Kras G12D inhibitor having a formula selected from formulae CF′ to CJ′, as disclosed herein, and the inhibitor of EGFR is selected from afatinib, erlotinib, gefitinib, lapatinib, cetuximab panitumumab, osimertinib, olnutinib, and EGF-816. In embodiments of the subject method, the Kras G12D inhibitor has a formula selected from formulae CF′ to CJ′, as disclosed herein, and the inhibitor of CDK4/6 is selected from palbociclib, ribociclib, and abemaciclib. In embodiments of the subject method, the Kras G12D inhibitor has the formula (CF′), as disclosed herein, and wherein the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CF′), as disclosed herein, and wherein the at least one inhibitor has a formula selected from formulae BB, BC, BC′, CF, CG, CH, CI, CJ, CK, and CK′. In embodiments of the subject method, the Kras G12D inhibitor has the formula (CF′), as disclosed herein, and wherein the at least one inhibitor has a formula selected from formulae CL to CZ and DA to DZ. In embodiments of the subject method, the Kras G12D inhibitor has the formula (CF′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has the formula (CF′), as disclosed herein, and the inhibitor of CDK4/6 is selected from palbociclib, ribociclib, and abemaciclib. In embodiments of the subject method, the Kras G12D inhibitor has the formula (CF′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CG′) or formula (CH′), as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CG′) or formula (CH′), as disclosed herein, and the at least one inhibitor has a formula selected from formulae CL to CZ and DA to DZ. In embodiments of the subject method, the Kras G12D inhibitor has the formula (CG′) or formula (CH′), as disclosed herein, and the inhibitor of EGFR is selected from afatinib, erlotinib, gefitinib, lapatinib, cetuximab panitumumab, osimertinib, olnutinib, and EGF-816. In embodiments of the subject method, the Kras G12D inhibitor has formula (CG′) or formula (CH′), as disclosed herein, and the inhibitor of CDK4/6 is selected from palbociclib, ribociclib, and abemaciclib. In embodiments of the subject method, the Kras G12D inhibitor has the formula (CI′) as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CI′), as disclosed herein, and the at least one inhibitor has a formula selected from formulae CL to CZ and DA to DZ. In embodiments of the subject method, the Kras G12D inhibitor has the formula (CI′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has formula (CI′), as disclosed herein, and the inhibitor of CDK4/6 is selected from palbociclib, ribociclib, and abemaciclib.

In embodiments of the subject method, the Kras G12D inhibitor has the formula (CJ′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CJ′), as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CJ′), as disclosed herein, and the at least one inhibitor has a formula selected from formulae CL to CZ and DA to DZ. In embodiments of the subject method, the Kras G12D inhibitor has the formula (CJ′), as disclosed herein, and the inhibitor of EGFR is selected from afatinib, erlotinib, gefitinib, lapatinib, cetuximab panitumumab, osimertinib, olnutinib, and EGF-816. In embodiments of the subject method, the Kras G12D inhibitor has the formula (CJ′), as disclosed herein, and the inhibitor of CDK4/6 is selected from palbociclib, ribociclib, and abemaciclib. In embodiments of the subject method, the Kras G12D inhibitor has the formula (CJ′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CH′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CH′), as disclosed herein, and the at least one inhibitor has a formula selected from formulae BB, BC, BC′, CF, CG, CH, CI, CJ, CK, and CK′.

In embodiments of the subject method, the Kras G12D inhibitor has the formula (CH′), as disclosed herein, and the at least one inhibitor has a formula selected from formulae CL to CZ and DA to DZ. In embodiments of the subject method, the Kras G12D inhibitor has the formula (CH′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has the formula (CH′), as disclosed herein, and the inhibitor of CDK4/6 is selected from palbociclib, ribociclib, and abemaciclib. In embodiments of the subject method, the Kras G12D inhibitor has the formula (CH′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CG′), as disclosed herein, and the at least one inhibitor has a formula selected from formulae CF to CZ, CK′, DA to DZ, A to F, N to Z, Z′, AA, AA′, BB, BC, and BC′. In embodiments of the subject method, the Kras G12D inhibitor has the formula (CG′), as disclosed herein, and the at least one inhibitor has a formula selected from formulae BB, BC, BC′, CF, CG, CH, CI, CJ, CK, and CK′. In embodiments of the subject method, the Kras G12D inhibitor has the formula (CG′), as disclosed herein, and the at least one inhibitor has a formula selected from formulae CL to CZ and DA to DZ. In embodiments of the subject method, the Kras G12D inhibitor has the formula (CG′), as disclosed herein, and the inhibitor of EGFR is selected from afatinib, erlotinib, gefitinib, lapatinib, cetuximab panitumumab, osimertinib, olmutinib, and EGF-816. In embodiments of the subject method, the Kras G12D inhibitor has the formula (CG′), as disclosed herein, and the inhibitor of CDK4/6 is selected from palbociclib, ribociclib, and abemaciclib. In embodiments of the subject method, the Kras G12D inhibitor has the formula (CG′), as disclosed herein, and the at least one inhibitor has a formula selected from formulae CF to CZ, CK′, DA to DZ, A to F, N to Z, Z′, AA, AA′, BB, BC, and BC′.

### Compositions

Another aspect of the present disclosure provides a composition comprising any of the inhibitors disclosed herein. The composition can be used for contacting a cell with one or more inhibitors as disclosed herein, e.g., in vitro, ex vivo, or in vivo. In some embodiments, the composition can be used for administering, to a subject (e.g., a human subject) in need thereof, one or more inhibitors as provided herein. For example, the composition can be administered to a subject in need thereof to treat and/or ameliorate symptoms of a wide variety of cancers in the subject. In some embodiments, the composition comprises one or more inhibitors capable of inhibiting cell signaling (e.g., cell proliferation signaling) in the cell, in accordance with the method of any one of the preceding claims. In some embodiments, the composition comprises one or more inhibitors capable of downregulating expression and/or activity of (a) a Ras protein (e.g., a mutated Ras protein, such as Kras G12D) and that of (b) one or more signaling molecules selected from Table 1, in accordance with the method of any one of the preceding claims. In some embodiments, the present disclosure provides a composition comprising: an inhibitor against (a) a Ras protein (e.g., a mutated Ras protein, such as Kras G12D); and/or an inhibitor against (b) one or more signaling molecules selected from Table 1. In some embodiments, the present disclosure provides a composition comprising: a gene encoding an inhibitor against (a) a Ras protein (e.g., a mutated Ras protein, such as Kras G12D); and/or a gene encoding an inhibitor against (b) one or more signaling molecules selected from Table 1. In some embodiments, the present disclosure provides a composition comprising: an inhibitor against (a) a Ras protein (e.g., a mutated Ras protein, such as Kras G12D); and/or an inhibitor against (b) one or more signaling molecules selected from Table 1.

In some embodiments, the present disclosure provides a composition comprising: (a) an inhibitor against a Ras G12D (e.g., KRas G12D) protein; and (b) an inhibitor against a Ras G12C (e.g., KRas G12C) protein. In some embodiments, the present disclosure provides a composition comprising: an inhibitor against (a) a Ras protein (e.g., a mutated Ras protein, such as Kras G12D), an inhibitor against (b) one or more signaling molecules selected from Table 1, and an inhibitor against a Ras G12C (e.g., KRas G12C) protein. In some cases, any of the composition disclosed herein can comprise delivery carriers (e.g., viral or non-viral delivery carriers) to deliver (i) any inhibitor and/or (ii) a gene encoding such inhibitor to the cell. Exemplary inhibitors of KRas G12C are known and may be included in the subject compositions and methods described herein. For example, included in subject compositions and methods are inhibitors of KRas G12C:

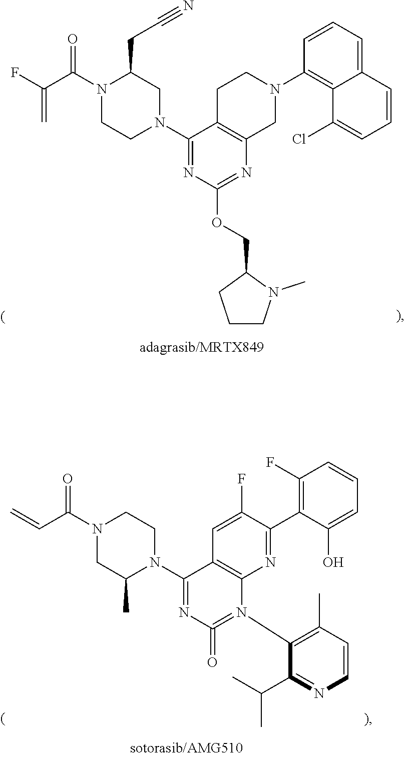

RMC-6291, RMC-6236, or JNJ-74699157 (ARS-3248). Exemplary inhibitors of SHP2 are known and may be included in the subject compositions and methods described herein. For example, included in subject compositions and methods are inhibitors of SHP2:

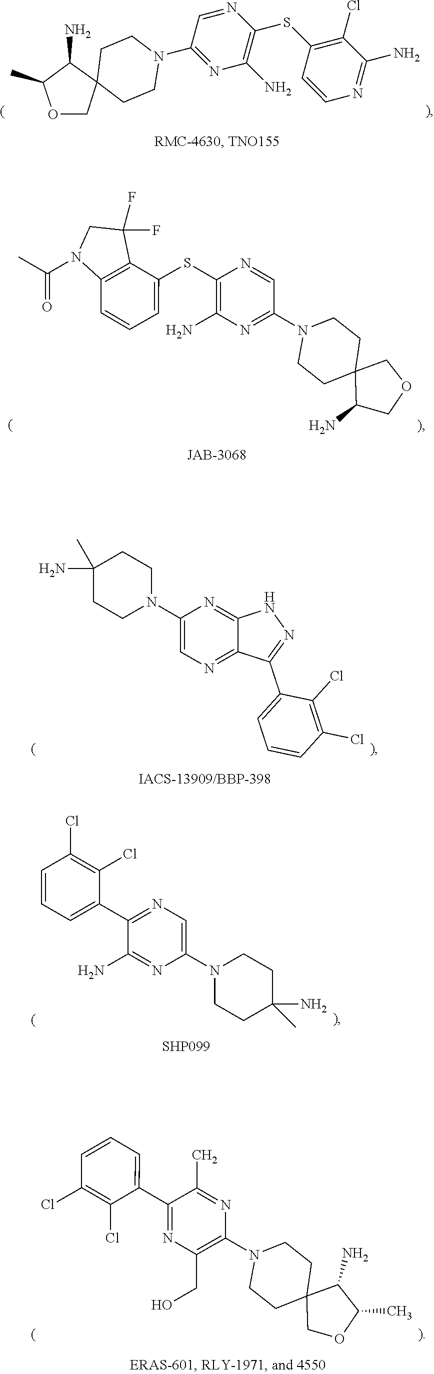 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

Exemplary inhibitors of SOS1 are known and may be included in the subject compositions and methods described herein. For example, included in subject compositions and methods are inhibitors of SOS1:

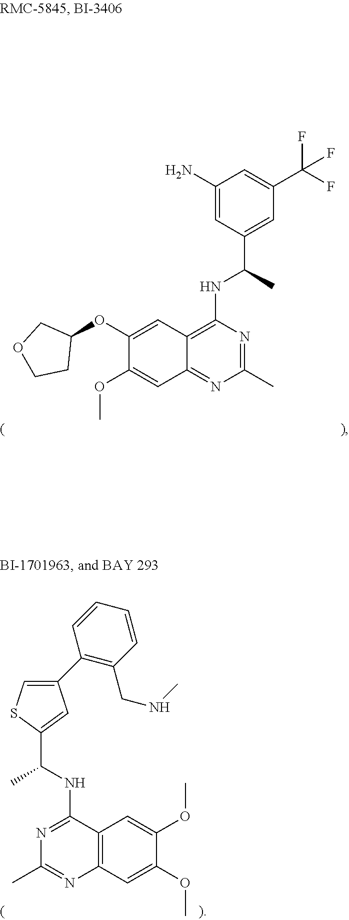

A composition can be formulated for any administration disclosed herein. In some embodiments, a composition can be formulated for infusion or oral administration. For example, a composition can be formulated in a liquid (e.g., saline) for infusion. In another example, a composition can be formulated in a unit dosage form, e.g., for oral administration, such as tablets, capsules, gel capsules, slow-release tablets, or the like. A unit dosage form as disclosed herein can comprise a unit dosage of a single agent (e.g., a single tablet having single therapeutic drug). Alternatively, a unit dosage form as disclosed herein can comprise a plurality of unit dosages of a plurality of agents (e.g., a single tablet comprising two therapeutic drugs).

### Compounds

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure can be utilized in the method section including methods of use and production disclosed herein, or vice versa.

Kras G12D Inhibitors

In embodiments of the subject methods, the methods include administering an inhibitor of KRAS G12D, including the molecules lacking electrophiles that are described in one or more of the following references: WO17/201161, WO19/099524, WO20/101736, WO20/047192, WO19/217307, WO20/146613, PCT/US2020/040254, WO20/055755, WO20/055758, WO20/055760, WO20/055756, WO20/055761, WO20/118066, WO21/061749, WO21/041671, or PCT/US2021/019678, all of which are herein incorporated by reference in their entirety for all uses. In embodiments of the subject methods, the methods include administering an inhibitor of KRAS G12D, including the molecules lacking electrophiles that are described in one or more of the following references: WO2018/119183, WO2018/217651, WO2019/051291, WO2019/217691, WO2019/241157, WO2019/213526, WO2019/213516, WO2018/143315, WO2020/027083, WO2020/027084, WO2018/206539, WO2019/110751, WO2019/215203, WO2019/155399, WO2019/150305, WO2020/081282, U.S. Pat. No. 10,968,214, WO2020/035031, WO2020/212895, CN111773225A, WO2014/206343, wo2016/165626, WO2018/007885, WO2021/058018, WO2021/055728, WO2021/057832, WO2021/052499, WO2021/043322, WO2021/037018, WO2021/031952, WO2020/050890, WO2020/106640, WO2017/080979, WO2020/178282, WO2019/141250, WO2020/259432, WO2016/176338, WO2015/184349, WO2020/163594, WO2020/163598, WO2020/132071, WO2020097537, WO2020177629, WO2020221239, WO2021023247, WO2020259573A1, WO2021027943. WO2021000885, WO2013177983, US20170000800, CN103450204, WO2020239123, WO2021081212; WO2021083167, WO2021084765, WO2021085653, or WO2021086833; all of which are herein incorporated by reference in their entirety for all uses.

In some embodiments, a KRas inhibitor (e.g., KRas G12D inhibitor and/or KRas G12C inhibitor) is capable of binding to one or more amino acid residues corresponding to a KRas Switch II Binding Pocket (e.g, WT or a mutant such as G12D or G12C). The KRas Switch II Binding Pocket comprises the KRas Switch II region, which comprises amino acid residues corresponding to KRas residues 60 to 76 of human WT KRas, and amino acid residues that interact with the KRas Switch II region amino acids (e.g., in a GTP bound form).

In embodiments the KRas Switch II Binding Pocket comprises amino acids corresponding to amino acids V7, V8, V9, G10, A11, G12, K16, P34, T58, A59, G60, Q61, E62, E63, Y64, S65, R68, D69, Y71, M72, F78, I92, H95, Y96, Q99, I100, R102, and V103 of human wildtype KRas. In embodiments the KRas Switch II Binding Pocket comprises amino acids of human WT KRas, human WT NRas, human WT HRas, or mutants thereof (e.g., D12 of KRas G12D mutant, C12, S12, V12, C13, D13, or V13 of the corresponding mutant Ras protein) corresponding to the amino acids of the KRas Switch II Binding Pocket. In embodiments the KRas Switch II Binding Pocket consists of amino acids corresponding to amino acids V7, V8, V9, G10, A11, G12, K16, P34, T58, A59, G60, Q61, E62, E63, Y64, S65, R68, D69, Y71, M72, F78, I92, H95, Y96, Q99, I100, R102, and V103 of human wildtype KRas. In some embodiments, the Switch II Binding Pocket of the GDP bound KRas protein comprises those amino acids that make up the Switch II Binding Pocket in the GTP bound form of KRas even though such amino acids may adopt a different conformation in the GDP bound form, or the amino acids of a mutant KRas or mutant or wildtype NRas or HRas that correspond to such amino acids.

In embodiments, the KRas Switch II Binding Pocket comprises amino acids V7, V9, G10, P34, T58, G60, Q61, E62, E63, R68, Y71, M72, Y96, Q99, and I100 of human wildtype KRas. In embodiments, the KRas Switch II Binding Pocket consists of amino acids corresponding to V7, V9, G10, P34, T58, G60, Q61, E62, E63, R68, Y71, M72, Y96, Q99, and I100 of human wildtype KRas. In some embodiments, a subject KRas inhibitor (e.g., KRas G12D inhibitor or KRas G12C inhibitor) utilized in practicing any of the methods disclosed herein or contained in any subject composition, is capable of binding or contacting one amino acid of the KRas Switch II Binding Pocket. In some embodiments, a subject KRas inhibitor (e.g., KRas G12D inhibitor or KRas G12C inhibitor) is capable of binding or contacting at least one amino acid of the KRas Switch II Binding Pocket. In some embodiments, a subject KRas inhibitor (e.g., KRas G12D inhibitor or KRas G12C inhibitor) is capable of binding or contacting multiple amino acids of the KRas Switch II Binding Pocket (e.g., 2, 3, 4, 5, 6, 7, 8, 9, 10, 11, 12, 13, or more amino acids). In some embodiments, a subject KRas inhibitor (e.g., KRas G12D inhibitor or KRas G12C inhibitor) is capable of binding or contacting the amino acid corresponding to V7 of wildtype human KRas. In some embodiments, a subject KRas inhibitor (e.g., KRas G12D inhibitor or KRas G12C inhibitor) is capable of binding or contacting the amino acid corresponding to V8 of wildtype human KRas. In some embodiments, a subject KRas inhibitor (e.g., KRas G12D inhibitor or KRas G12C inhibitor) is capable of binding or contacting the amino acid corresponding to V9 of wildtype human KRas. In some embodiments, a subject KRas inhibitor (e.g., KRas G12D inhibitor or KRas G12C inhibitor) is capable of binding or contacting the amino acid corresponding to G10 of wildtype human KRas. In some embodiments, a subject KRas inhibitor (e.g., KRas G12D inhibitor or KRas G12C inhibitor) is capable of binding or contacting the amino acid corresponding to A11 of wildtype human KRas. In some embodiments, a subject KRas inhibitor (e.g., KRas G12D inhibitor or KRas G12C inhibitor) is capable of binding or contacting the amino acid corresponding to G12 of wildtype human KRas (e.g, G12D, G12C, or other mutants). In some embodiments, a subject KRas inhibitor (e.g., KRas G12D inhibitor or KRas G12C inhibitor) is capable of binding or contacting the amino acid corresponding to K16 of wildtype human KRas. In some embodiments, a subject KRas inhibitor (e.g., KRas G12D inhibitor or KRas G12C inhibitor) is capable of binding or contacting the amino acid corresponding to P34 of wildtype human KRas. In some embodiments, a subject KRas inhibitor (e.g., KRas G12D inhibitor or KRas G12C inhibitor) is capable of binding or contacting the amino acid corresponding to T58 of wildtype human KRas. In some embodiments, a subject KRas inhibitor (e.g., KRas G12D inhibitor or KRas G12C inhibitor) is capable of binding or contacting the amino acid corresponding to A59 of wildtype human KRas. In some embodiments, a subject KRas inhibitor (e.g., KRas G12D inhibitor or KRas G12C inhibitor) is capable of binding or contacting the amino acid corresponding to G60 of wildtype human KRas. In some embodiments, a subject KRas inhibitor (e.g., KRas G12D inhibitor or KRas G12C inhibitor) is capable of binding or contacting the amino acid corresponding to Q61 of wildtype human KRas. In some embodiments, a subject KRas inhibitor (e.g., KRas G12D inhibitor or KRas G12C inhibitor) is capable of binding or contacting the amino acid corresponding to E62 of wildtype human KRas. In some embodiments, a subject KRas inhibitor (e.g., KRas G12D inhibitor or KRas G12C inhibitor) is capable of binding or contacting the amino acid corresponding to E63 of wildtype human KRas. In some embodiments, a subject KRas inhibitor (e.g., KRas G12D inhibitor or KRas G12C inhibitor) is capable of binding or contacting the amino acid corresponding to Y64 of wildtype human KRas. In some embodiments, a subject KRas inhibitor (e.g., KRas G12D inhibitor or KRas G12C inhibitor) is capable of binding or contacting the amino acid corresponding to S65 of wildtype human KRas. In some embodiments, a subject KRas inhibitor (e.g., KRas G12D inhibitor or KRas G12C inhibitor) is capable of binding or contacting the amino acid corresponding to R68 of wildtype human KRas. In some embodiments, a subject KRas inhibitor (e.g., KRas G12D inhibitor or KRas G12C inhibitor) is capable of binding or contacting the amino acid corresponding to D69 of wildtype human KRas. In some embodiments, a subject KRas inhibitor (e.g., KRas G12D inhibitor or KRas G12C inhibitor) is capable of binding or contacting the amino acid corresponding to Y71 of wildtype human KRas. In some embodiments, a KRas inhibitor (e.g., KRas G12D inhibitor or KRas G12C inhibitor) is capable of binding or contacting the amino acid corresponding to M72 of wildtype human KRas. In some embodiments, a subject KRas inhibitor (e.g., KRas G12D inhibitor or KRas G12C inhibitor) is capable of binding or contacting the amino acid corresponding to F78 of wildtype human KRas. In some embodiments, a KRas inhibitor (e.g., KRas G12D inhibitor or KRas G12C inhibitor) is capable of binding or contacting the amino acid corresponding to I92 of wildtype human KRas. In some embodiments, a subject KRas inhibitor (e.g., KRas G12D inhibitor or KRas G12C inhibitor) is capable of binding or contacting the amino acid corresponding to H95 of wildtype human KRas. In some embodiments, a subject KRas inhibitor (e.g., KRas G12D inhibitor or KRas G12C inhibitor) is capable of binding or contacting the amino acid corresponding to Y96 of wildtype human KRas. In some embodiments, a KRas inhibitor (e.g., KRas G12D inhibitor or KRas G12C inhibitor) is capable of binding or contacting the amino acid corresponding to Q99 of wildtype human KRas.

In some embodiments, a subject KRas inhibitor (e.g., KRas G12D inhibitor or KRas G12C inhibitor) is capable of binding or contacting the amino acid corresponding to I100 of wildtype human KRas. In some embodiments, a subject KRas inhibitor (e.g., KRas G12D inhibitor or KRas G12C inhibitor) is capable of binding or contacting the amino acid corresponding to R102 of wildtype human KRas. In some embodiments, a subject KRas inhibitor (e.g., KRas G12D inhibitor or KRas G12C inhibitor) is capable of binding or contacting the amino acid corresponding to V103 of wildtype human KRas.

In some embodiments, a subject Kras G12D inhibitor selectively inhibits Kras G12D activity and/or expression relatively to a wildtype Kras (without such point mutation at the 12 residue). For example, selective inhibition can be evidenced by a difference in IC50 values of a given Kras G12D inhibitor against the mutant Kras G12D relative to wildtype Kras. In some embodiment, a subject Kras G12D inhibitor exhibits an IC50 (either biochemical or cellular IC50) that is at least 1×, 2×, 3×, 4×, 5×, 10×, 20×, 50×, 100×, or 1000×, lower than that of the IC50 value against the wildtype Kras. For example, a subject Kras G12D inhibitor exhibits an IC50 of 5 nM against Kras G12D is 100× lower than its IC50 against a wildtype at 500 nM, indicating a high degree of selectivity against Kras G12D.

In embodiments, the KRAS G12D inhibitor has the formula (CA) immediately below, or a pharmaceutically acceptable salt or solvate thereof:

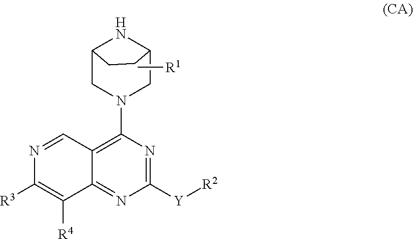

or a pharmaceutically acceptable salt thereof; wherein:
R¹ is hydrogen, hydroxy, halogen, C₁-C₃ alkyl, C₁-C₃ cyanoalkyl, C₁-C₃ hydroxyalkyl, HC(═O)—, —CO₂R₅, —CO₂N(R⁵)₂ or a 5-6 membered heteroaryl;
Y is a bond, O or NR⁵;
R² is hydrogen, —N(R⁵)₂, heterocyclyl, C₁-C₆ alkyl, -L-heterocyclyl, -L-aryl, -L-heteroaryl, -L-cycloalkyl, -L-N(R⁵)₂, -L-NHC(═NH)NH₂, -L-C(O)N(R⁵)₂, -L-C₁-C₆ haloalkyl, -L-OR⁵, -L-(CH₂OR⁵)(CH₂)nOR⁵, -L-NR⁵C(O)-aryl, -L-COOH, or -LC(═O)OC₁-C₆ alkyl, wherein the heterocyclyl and the aryl portion of -L-NR⁵C(O)-aryl and the heterocyclyl portion of -L-heterocyclyl and the cycloalkyl portion of the -L-cycloalkyl may be optionally substituted with one or more R⁶, and wherein the aryl or heteroaryl of the -L-aryl and the -L-heteroaryl may be optionally substituted with one or more R⁷;
each L is independently a C₁-C₄ alkylene optionally substituted with hydroxy, C₁-C₄ hydroxyalkyl or heteroaryl;
R³ is aryl or heteroaryl, wherein the aryl or the heteroaryl is optionally substituted with one or more R⁸;
R⁴ is hydrogen, halogen or C₁-C₃ alkyl;
each R⁵ is independently hydrogen or C₁-C₃ alkyl;
each R⁶ is independently halogen, hydroxy, C₁₋₆ C₃ hydroxyalkyl, C₁-C₃ alkyl, C₁-C₃ haloalkyl, C₁-C₃ alkoxy, cyano, -Q-phenyl, -Q-phenylSO₂F, —NHC(O)phenyl, —NHC(O)phenylSO₂F, C₁-C₃ alkyl substituted pyrazolyl, araC₁-C₃ alkyl-, tert-butyldimethylsilyloxyCH₂—, —N(R⁵)₂, (C₁-C₃ alkoxy) C₁-C₃ alkyl-, (C₁-C₃ alkyl)C(═O), oxo, (C₁-C₃ haloalkyl)C(═O)—, —SO₂F, (C₁-C₃ alkoxy) C₁-C₃ alkoxy, —CH₂OC(O)N(R⁵)₂, —CH₂NHC(O)OC₁-C₆ alkyl, —CH₂NHC(O)N(R⁵)₂, —CH₂NHC(O)C₁-C₆ alkyl, —CH₂(pyrazolyl), —CH₂NHSO₂ C₁-C₆ alkyl, —CH₂OC(O)heterocyclyl, —OC(O)N(R⁵)₂, —OC(O)NH(C₁₋₆ C₃ alkyl)O(C₁-C₃ alkyl), —OC(O)NH(C₁₋₆ C₃ alkyl)O(C₁₋₆ C₃ alkyl)phenyl(C₁₋₆ C₃ alkyl)N(CH₃)₂, —OC(O)NH(C₁₋₆ C₃ alkyl)O(C₁₋₆ C₃ alkyl)phenyl or —OC(O)heterocyclyl, —CH₂heterocyclyl, wherein the phenyl of —NHC(O)phenyl or —OC(O)NH(C₁₋₆ C₃ alkyl)O(C₁-C₃ alkyl)phenyl is optionally substituted with —C(O)H or OH and wherein the heterocyclyl of —CH₂heterocyclyl is optionally substituted with oxo;
Q is a bond or 0;
each R⁷ is independently halogen, hydroxy, HC(═O)—, C₁-C₄ alkyl, C₁-C₄ alkoxy, C₁-C₄ haloalkyl, C₁-C₄ hydroxyalkyl, or —N(R⁵)₂; and
each R⁸ is independently halogen, cyano, hydroxy, C₁-C₄ alkyl, —S— C₁-C₃ alkyl, C₂-C₄ alkenyl, C₂-C₄ alkynyl, C₂-C₄ hydroxyalkynyl, C₁-C₃ cyanoalkyl, triazolyl, C₁-C₃ haloalkyl, —O— C₁-C₃ haloalkyl, —S— C₁-C₃ haloalkyl, C₁-C₃ alkoxy, hydroxy C₁-C₃ alkyl, —CH₂C(═O)N(R⁵)₂, —C₃-C₄ alkynyl(NR⁵)₂, —N(R⁵)₂, deutero C₂-C₄ alkynyl, (C₁-C₃ alkoxy)halo C₁-C₃ alkyl-, or C₃-C₆ cycloalkyl wherein said C₃-C₆ cycloalkyl is optionally substituted with halogen or C₁-C₃ alkyl;
the term “cycloalkyl” as employed in this embodiment includes saturated and partially unsaturated cyclic hydrocarbon groups wherein the cycloalkyl group additionally is optionally substituted as defined herein, the term “cycloalkyl” as used in this embodiment also includes bridged cycloalkyls; an “aryl” group as used in this embodiment is a C₆-C₁₄ aromatic moiety comprising one to three aromatic rings, which is optionally substituted with one or more substituents as defined herein, “Aryl” as used in this embodiment also refers to bicyclic or tricyclic ring systems in which one or two rings, respectively, of said aryl ring system may be saturated or partially saturated, and wherein if said ring system includes two saturated rings, said saturated rings may be fused or spirocyclic; the term “heterocyclyl” or “heterocyclic” as used in this embodiment refers to a ring structure wherein one or more atoms are selected from the group consisting of N, O, and S wherein the ring N atom may be oxidized to N—O, and the ring S atom may be oxidized to SO or SO₂, the remainder of the ring atoms being carbon, the heterocyclyl of this embodiment may be a monocyclic, a bicyclic, a spirocyclic or a bridged ring system and the heterocyclic group of this embodiment is optionally substituted at one or more positions; as used in this embodiment, the term “heteroaryl” refers to groups having 5 to 14 ring atoms, having 6, 10, or 14 p electrons shared in a cyclic array; and having, in addition to carbon atoms, from one to three heteroatoms per ring selected from the group consisting of N, O, and S, “Heteroaryl” as used in this embodiment also refers to bicyclic ring systems having, in addition to carbon atoms, from one to three heteroatoms per ring selected from the group consisting of N, O, and S in which one ring system may be saturated or partially saturated. In embodiments, the KRAS G12D inhibitor is a compound shown below:

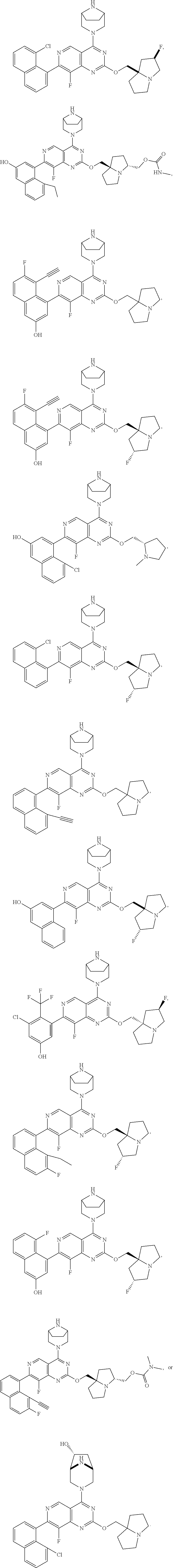

In embodiments, the inhibitor of KRAS G12D and/or KRAS G12C is a compound having the formula (CB):

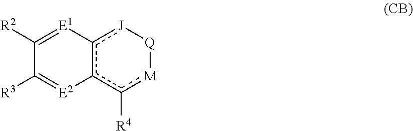

wherein E¹ and E² are each independently N or CR¹;
J is N, NR¹⁰, or CR¹⁰;
M is N, NR¹³, or CR¹³; is a single or double bond as necessary to give every atom its normal valence;
R¹ is independently H, hydroxy, C₁₋₆alkyl, C₁₋₄haloalkyl, C₁₋₄alkoxy, NH—C₁₋₄alkyl, N(C₁₋₆alkyl)₂, cyano, or halo;
R² is halo, C₁₋₆alkyl, C₁₋₆haloalkyl, OR′, N(R′)2, C₂₋₃alkenyl, C₂₋₃alkynyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or C₀₋₃alkylene-C₂₋₁₄heteroaryl,
   and each R′ is independently H, C₁₋₆alkyl, C₁₋₆haloalkyl, C₃₋₁₄cycloalkyl, C₂₋₁₄heterocycloalkyl, C₂₋₃alkenyl, C₂₋₃alkynyl, aryl, or heteroaryl,
   or two R′ substituents, together with the nitrogen atom to which they are attached, form a 3-7-membered ring;
R³ is halo, C₁₋₆alkyl, C₁₋₆haloalkyl, C₁₋₄alkoxy, C₃₋₄cycloalkyl, C₂₋₁₄heterocycloalkyl, C₂₋₃alkenyl, C₂₋₃alkynyl, aryl, or heteroaryl;
   R⁴ is

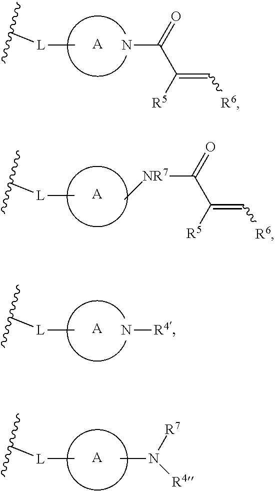

ring A is a monocyclic 4-7 membered ring or a bicyclic, bridged, fused, or spiro 6-11 membered ring;
L is a bond, C₁₋₆alkylene, —O—C₀₋₅alkylene, —S—C₀₋₅alkylene, or —NH—C₀₋₅alkylene, and for C₂₋₆alkylene, —O— C₂₋₅alkylene, —S— C₂₋₅alkylene, and NH—C₂₋₅alkylene,
   one carbon atom of the alkylene group can optionally be replaced with O, S, or NH;
R^{4′} is H, C₁₋₅alkyl, C₂₋₅alkynyl, C₁₋₆alkylene-O— C₁₋₄alkyl, C₁₋₆alkylene-OH, C₁₋₆haloalkyl, cycloalklyl, heterocycloalkyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or selected from

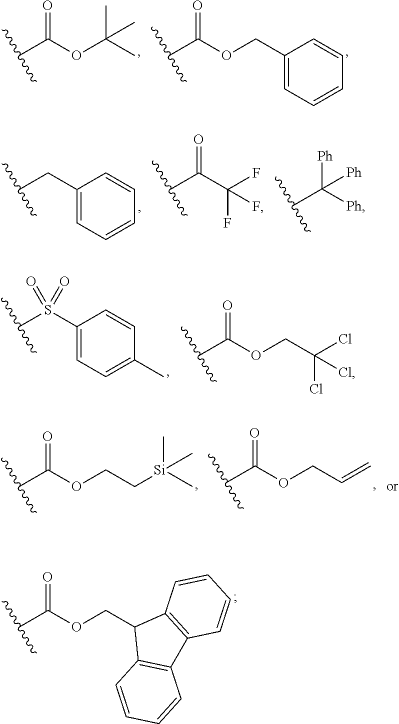

R⁵ and R⁶ are each independently H, halo, C₁₋₆alkyl, C₂₋₆alkynyl, C₁₋₆alkylene-O—C₁₋₄alkyl, C₁₋₆alkylene-OH, C₁₋₆haloalkyl, C₁₋₆ alkyleneamine, C₀₋₆alkylene-amide, C₀₋₃alkylene-C(O)OH, C₀₋₃alkylene-C(O)OC₁₋₄alkyl, C₁₋₆alkylene-O-aryl, C₀₋₃alkylene-C(O)C₁₋₄alkylene-OH, cycloalkyl, heterocycloalkyl, aryl, heteroaryl, C₀₋₃alkylene-C₃₋₁₄cycloalkyl, C₀₋₃alkylene-C₂₋₁₄heterocycloalkyl, C₀₋₃alkylene-C₆₋₁₄aryl, C₀₋₃alkylene-C₂₋₁₄heteroaryl, or cyano, or R⁵ and R⁶, together with the atoms to which they are attached, form a 4-6 membered ring;
R⁷ is H or C₁₋₆alkyl,
   or R⁷ and R⁵, together with the atoms to which they are attached, form a 4-6 membered ring;
Q is CR⁸R⁹, C═CR⁸R⁹, C═O, C═S, or C═NR⁸;
R⁸ and R⁹ are each independently H, C₁₋₆alkyl, hydroxy, C₁₋₆alkoxy, cyano, nitro, or C₃₋₆cycloalkyl, or R⁸ and R⁹, taken together with the carbon atom to which they are attached, can form a 3-6 membered ring;
R¹⁰ is C₁₋₆alkyl, C₀₋₃alkylene-C₆₋₁₄aryl, C₀₋₃alkylene-C₃₋₁₄heteroaryl, C₀₋₃alkylene-C₃₋₁₄cycloalkyl, C₀₋₃alkylene-C₂₋₁₄heterocycloalkyl, C₁₋₆alkoxy, O—C₀₋₃alkylene-C₆₋₁₄aryl, O—C₀₋₃alkylene-C₃₋₁₄heteroaryl, O—C₀₋₃alkylene-C₃₋₁₄cycloalkyl, O—C₀₋₃alkylene-C₂₋₁₄heterocycloalkyl, NH—C₁₋₆alkyl, N(C₁₋₈alkyl)₂, NH—C₀₋₃alkylene-C₆₋₁₄aryl, NH— C₀₋₃alkylene-C₂₋₁₄heteroaryl, NH—C₀₋₃alkylene-C₃₋₁₄cycloalkyl, NH—C₀₋₃alkylene-C₂₋₁₄heterocycloalkyl, halo, cyano, or C₁₋₆alkylene-amine; and
R¹³ is C₁₋₆alkyl, C₁₋₆haloalkyl, C₁₋₆alkyleneamine, or C₃₋₁₄cycloalkyl,
or a pharmaceutically acceptable salt thereof,
   with the proviso that
   (1) when J is NR¹⁰, M is N or CR¹³;
   (2) when M is NR¹³, J is N or CR¹⁰;
   (3) when J is CR¹⁰, M is N or NR¹³; and
   (4) when M is CR¹³, J is N or NR¹⁰;
as used in this embodiment, the term “aryl” refers to as monocyclic or polycyclic aromatic group. As used in this embodiment, aryl also refers to bicyclic and tricyclic carbon rings, where one ring is aromatic and the others are saturated, partially unsaturated, or aromatic. As used in this embodiment, unless otherwise indicated, an aryl group can be unsubstituted or substituted with one or more, and in particular one to four, groups independently selected from, for example, halo, C₁₋₆₁alkyl, C₂₋₈alkenyl, C₂₋₈alkynyl, —CF₃, —OCF₃, —NO₂, —CN, —NC, —OH, alkoxy, amino, —CO₂H, —CCC₁₋₆alkyl, —OCOC₁₋₆alkyl, C₃₋₁₀cycloalkyl, C₃₋₁₀heterocycloalkyl, C₆₋₁₀aryl, and C₅₋₁₀heteroaryl. As used in this embodiment, the term “cycloalkyl” refers to a monocyclic or polycyclic non-aromatic carbocyclic ring, where the polycyclic ring can be fused, bridged, or spiro. As used in this embodiment, the term “heterocycloalkyl” means a monocyclic or poly cyclic (e.g., bicyclic), saturated or partially unsaturated, ring system containing 3 or more (e.g., 3 to 12, 4 to 10, 4 to 8, or 5 to 7) total atoms, of which one to five (e.g., 1, 2, 3, 4, or 5) of the atoms are independently selected from nitrogen, oxygen, and sulfur. As used in this embodiment, unless otherwise indicated, a cycloalkyl or heterocycloalkyl group can be unsubstituted or substituted with one or more, and in particular one to four, groups. Some contemplated substituents include halo, C₁₋₆alkyl, C₂₋₈alkenyl, C₂₋₈alkynyl, —CF₃, —OCF₃, —NO₂, —CN, —NC, —OH, alkoxy, amino, CO₂H, —CCC₁₋₆alkyl, —OOCC₁₋₆alkyl, C₃₋₁₀cycloalkyl, C₃₋₁₀heterocycloalkyl, C₆₋₁₀aryl, and C₅₋₁₀heteroaryl. As used in this embodiment, the term “heteroaryl” refers to a monocyclic or polycyclic ring system (for example, bicyclic) containing one to three aromatic rings and containing one to four (e.g., 1, 2, 3, or 4) heteroatoms selected from nitrogen, oxygen, and sulfur in an aromatic ring. As used in this embodiment, heteroaryl also refers to bicyclic and tricyclic rings, where one ring is aromatic and the others are saturated, partially unsaturated, or aromatic. As used in this embodiment, unless otherwise indicated, a heteroaryl group can be unsubstituted or substituted with one or more, and in particular one to four or one or two, substituents. Contemplated substituents include halo, C₁₋₆alkyl, C₂₋₈alkenyl, C₂₋₈alkynyl, —CF₃, —OCF₃, —NO₂, —CN, —NC, —OH, alkoxy, amino, —CO₂H, —CCC₁₋₆alkyl, —OCOC₁₋₆alkyl, C₃₋₁₀cycloalkyl, C₃₋₁₀heterocycloalkyl, C₆₋₁₀aryl, and C₅₋₁₀heteroaryl.
   In embodiments, the compound is

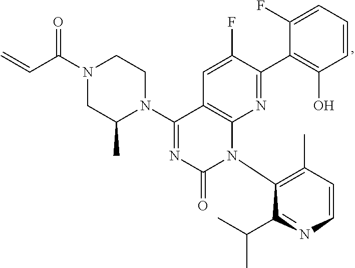

or a pharmaceutically acceptable salt or solvate thereof. In embodiments, the compound is

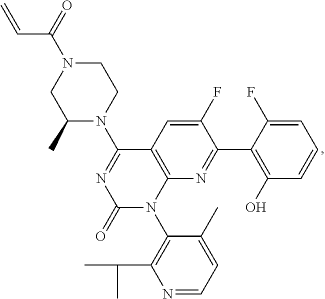 [SMILES: C=CC(=O)N1CCN(c2nc(=O)n(-c3c(C)ccnc3C(C)C)c3nc(-c4c(O)cccc4F)c(F)cc23)[C@@H](C)C1]

or a pharmaceutically acceptable salt or solvate thereof.

In embodiments, the inhibitor of KRAS G12D is a compound having the formula (CC):

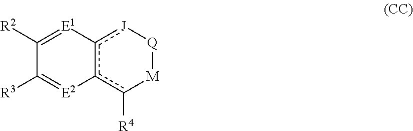

wherein E¹ and E² are each independently N or CR¹;
J is N, NR¹⁰, or CR¹⁰;
M is N, NR¹³, or CR¹³; is a single or double bond as necessary to give every atom its normal valence;
R¹ is independently H, hydroxy, C₁₋₆alkyl, C₁₋₄haloalkyl, C₁₋₄alkoxy, NH—C₁₋₄alkyl, N(C₁₋₆alkyl)₂, cyano, or halo;
R² is halo, C₁₋₆alkyl, C₁₋₆haloalkyl, OR′, N(R′)₂, C₂₋₃alkenyl, C₂₋₃alkynyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or C₀₋₃alkylene-C₂₋₁₄heteroaryl,
   and each R′ is independently H, C₁₋₆alkyl, C₁₋₆haloalkyl, C₃₋₁₄cycloalkyl, C₂₋₁₄heterocycloalkyl, C₂₋₃alkenyl, C₂₋₃alkynyl, aryl, or heteroaryl,
   or two R′ substituents, together with the nitrogen atom to which they are attached, form a 3-7-membered ring;
R³ is halo, C₁₋₆alkyl, C₁₋₆haloalkyl, C₁₋₄alkoxy, C₃₋₄cycloalkyl, C₂₋₁₄heterocycloalkyl, C₂₋₃alkenyl, C₂₋₃alkynyl, aryl, or heteroaryl;
R⁴ is

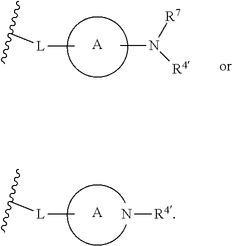

ring A is a monocyclic 4-7 membered ring or a bicyclic, bridged, fused, or spiro 6-11 membered ring;
L is a bond, C₁₋₆alkylene, —O—C₀₋₅alkylene, —S—C₀₋₅alkylene, or —NH—C₀₋₅alkylene, and for C₂₋₆alkylene, —O— C₂₋₅alkylene, —S— C₂₋₅alkylene, and NH—C₂₋₅alkylene, one carbon atom of the alkylene group can optionally be replaced with O, S, or NH;
R^{4′} is H, C₁₋₅alkyl, C₂₋₅alkynyl, C₁₋₆alkylene-O— C₁₋₄alkyl, C₁₋₆alkylene-OH, C₁₋₆haloalkyl, cycloalklyl, heterocycloalkyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or selected from

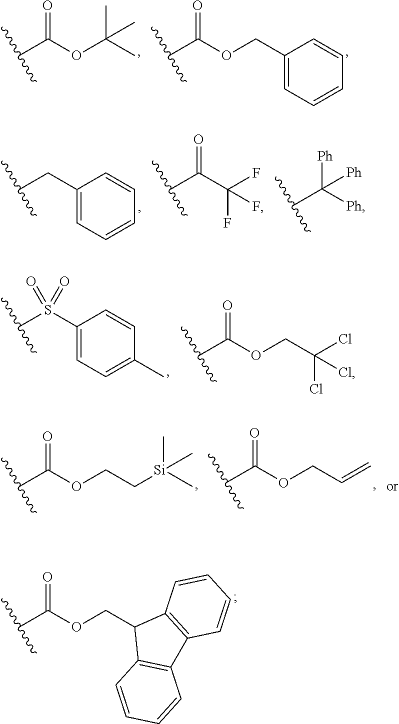

R⁵ and R⁶ are each independently H, halo, C₁₋₆alkyl, C₂₋₆alkynyl, C₁₋₆alkylene-O—C₁₋₄alkyl, C₁₋₆alkylene-OH, C₁₋₆haloalkyl, C₁₋₆ alkyleneamine, C₀₋₆alkylene-amide, C₀₋₃alkylene-C(O)OH, C₀₋₃alkylene-C(O)OC₁₋₄alkyl, C₁₋₆alkylene-O-aryl, C₀₋₃alkylene-C(O)C₁₋₄alkylene-OH, cycloalkyl, heterocycloalkyl, aryl, heteroaryl, C₀₋₃alkylene-C₃₋₁₄cycloalkyl, C₀₋₃alkylene-C₂₋₁₄heterocycloalkyl, C₀₋₃alkylene-C₆₋₁₄aryl, C₀₋₃alkylene-C₂₋₁₄heteroaryl, or cyano,
   or R⁵ and R⁶, together with the atoms to which they are attached, form a 4-6 membered ring;
R⁷ is H or C₁₋₆alkyl,
   or R⁷ and R⁵, together with the atoms to which they are attached, form a 4-6 membered ring;
Q is CR⁸R⁹, C═CR⁸R⁹, C═O, C═S, or C═NR⁸;
R⁸ and R⁹ are each independently H, C₁₋₆alkyl, hydroxy, C₁₋₆alkoxy, cyano, nitro, or C₃₋₆cycloalkyl, or R⁸ and R⁹, taken together with the carbon atom to which they are attached, can form a 3-6 membered ring;
R¹⁰ is C₁₋₆alkyl, C₀₋₃alkylene-C₆₋₁₄aryl, C₀₋₃alkylene-C₃₋₁₄heteroaryl, C₀₋₃alkylene-C₃₋₁₄cycloalkyl, C₀₋₃alkylene-C₂₋₁₄heterocycloalkyl, C₁₋₆alkoxy, O—C₀₋₃alkylene-C₆₋₁₄aryl, O—C₀₋₃alkylene-C₃₋₁₄heteroaryl, O—C₀₋₃alkylene-C₃₋₁₄cycloalkyl, O—C₀₋₃alkylene-C₂₋₁₄heterocycloalkyl, NH—C₁₋₆alkyl, N(C₁₋₈alkyl)₂, NH—C₀₋₃alkylene-C₆₋₁₄aryl, NH— C₀₋₃alkylene-C₂₋₁₄heteroaryl, NH—C₀₋₃alkylene-C₃₋₁₄cycloalkyl, NH—C₀₋₃alkylene-C₂₋₁₄heterocycloalkyl, halo, cyano, or C₁₋₆alkylene-amine; and
R¹³ is C₁₋₆alkyl, C₁₋₆haloalkyl, C₁₋₆alkyleneamine, or C₃₋₁₄cycloalkyl,
or a pharmaceutically acceptable salt thereof,
   with the proviso that
   (1) when J is NR¹⁰, M is N or CR¹³;
   (2) when M is NR¹³, J is N or CR¹⁰;
   (3) when J is CR¹⁰, M is N or NR¹³; and
   (4) when M is CR¹³, J is N or NR¹⁰;
as used in this embodiment, the term “aryl” refers to a monocyclic or polycyclic aromatic group. As used in this embodiment, aryl also refers to bicyclic and tricyclic carbon rings, where one ring is aromatic and the others are saturated, partially unsaturated, or aromatic. As used in his embodiment, unless otherwise indicated, an aryl group can be unsubstituted or substituted with one or more, and in particular one to four, groups independently selected from, For example, halo, C₁₋₆alkyl, C₂₋₈alkenyl, C₂₋₈alkynyl, —CF₃, —OCF₃, —NO₂, —CN, —NC, —OH, alkoxy, amino, —CO₂H, —CCC₁₋₆alkyl, —OCOC₁₋₆alkyl, C₃₋₁₀cycloalkyl, C₃₋₁₀heterocycloalkyl, C₆₋₁₀aryl, and C₅₋₁₀heteroaryl. As used in this embodiment, the term “cycloalkyl” refers to a monocyclic or polycyclic non-aromatic carbocyclic ring, where the polycyclic ring can be fused, bridged, or spiro. As used in this embodiment, the term “heterocycloalkyl” means a monocyclic or poly cyclic (e.g, bicyclic), saturated or partially unsaturated, ring system containing 3 or more (e.g., 3 to 12, 4 to 10, 4 to 8, or 5 to 7) total atoms, of which one to five (e.g., 1, 2, 3, 4, or 5) of the atoms are independently selected from nitrogen, oxygen, and sulfur. As used in this embodiment, unless otherwise indicated, a cycloalkyl or heterocycloalkyl group can be unsubstituted or substituted with one or more, and in particular one to four, groups. Some contemplated substituents include halo, C₁₋₆alkyl, C₂₋₈alkenyl, C₂₋₈alkynyl, —CF₃, —OCF, —NO₂, —CN, —NC, —OH, alkoxy, amino, CO₂H, —CCC₁₋₆alkyl, —OCOC₁₋₆alkyl, C₃₋₁₀cycloalkyl, C₃₋₁₀heterocycloalkyl, C₆₋₁₀aryl, and C₅₋₁₀heteroaryl. As used in this embodiment, the term “heteroaryl” refers to a monocyclic or polycyclic ring system (for example, bicyclic) containing one to three aromatic rings and containing one to four (e.g., 1, 2, 3, or 4) heteroatoms selected from nitrogen, oxygen, and sulfur in an aromatic ring. As used in this embodiment, heteroaryl also refers to bicyclic and tricyclic rings, where one ring is aromatic and the others are saturated, partially unsaturated, or aromatic. As used in this embodiment, unless otherwise indicated, a heteroaryl group can be unsubstituted or substituted with one or more, and in particular one to four or one or two, substituents. Contemplated substituents include halo, C₁₋₆alkyl, C₂₋₈alkenyl, C₂₋₈alkynyl, —CF₃, —OCF₃, —NO₂, —CN, —NC, —OH, alkoxy, amino, —CO₂H, —CCC₁₋₆alkyl, —OCOC₁₋₆alkyl, C₃₋₁₀cycloalkyl, C₃₋₁₀heterocycloalkyl, C₆₋₁₀aryl, and C₅₋₁₀heteroaryl.
   In embodiments, the compound is

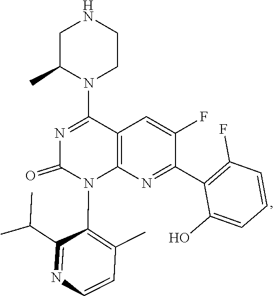

or a pharmaceutically acceptable salt or solvate thereof. In embodiments, the compound is

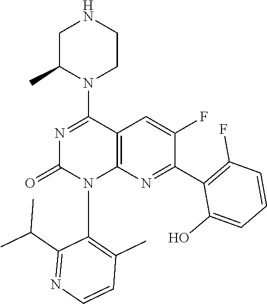 [SMILES: Cc1ccnc(C(C)C)c1-n1c(=O)nc(N2CCNC[C@@H]2C)c2cc(F)c(-c3c(O)cccc3F)nc21]

or a pharmaceutically acceptable salt or solvate thereof.

In embodiments, the inhibitor of KRAS G12D and/or KRAS G12C is a compound having a structure of the formula (CD) immediately below;

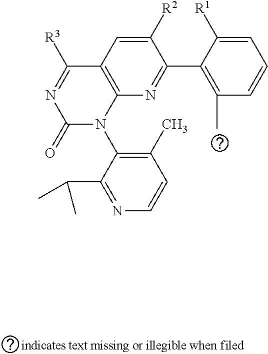

wherein
R¹ is H, halo, or —CH₃;
R² is H, halo, or —CH₃;
   R³ is

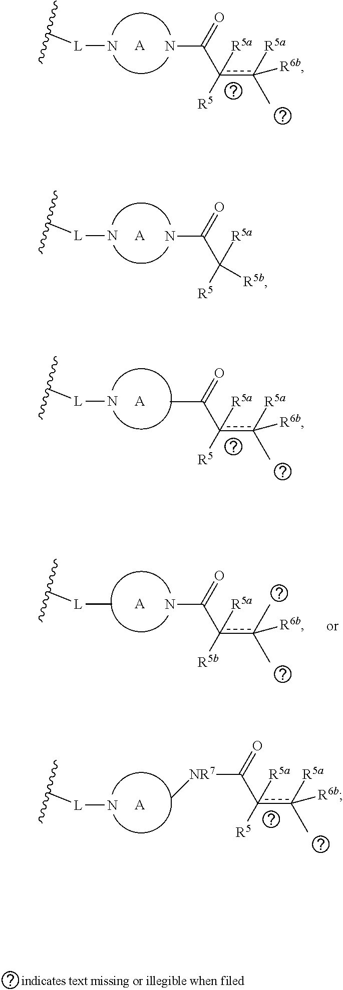

b is optionally a single or a double bond;
ring A is a monocyclic 4-7 membered ring or a bicyclic, bridged, fused, or spiro 6-11 membered ring;
L is a bond or NR⁴;
R⁴ is H, —C₁₋₆alkyl, —C₂₋₆alkynyl, C₁₋₆alkylene-O—C₁₋₄alkyl, C₁₋₆alkylene-OH, C₁₋₆haloalkyl, —C₁₋₆alkyleneamine, —C₀₋₆alkylene-amide, —C(O)OH, —C(O)OC₁₋₄alkyl, —C₁₋₆alkylene-O-aryl, —N═N, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R⁵ is H, halo, an —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, —C₀₋₃alkylene-C₂₋₁₄heteroaryl, or cyano;
R^{5a} is selected from H, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R^{5b} is selected from H, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
or R^{5a} and R^{5b} together, may represent an ═O or ═N═N;
R⁶ is H, halo, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene- amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R^{5a} and R^{6a}, together with the atoms to which they are attached, may form a 3-6 membered ring that optionally includes one or two heteroatoms selected from 0, S or N; or
R^{5a} and R^{6a} are absent when b is a double bond;
R^{6a} is H, or —C₁₋₆alkyl;
R^{6b} is H, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₁₋₆alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, —C₀₋₃alkylene-C₂₋₁₄heteroaryl, or cyano;
or R^{6a} and R^{6b} together, may represent an ═O;
R⁷ is H or C₁₋₈alkyl;
R⁸ is H, OH, NR^{a}R^{b};
   wherein R^{a} and R^{b} are each independently H, halo, —C₁₋₆alkyl, —C₂₋₆alkynyl;
   wherein the ring A or the —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆ alkyleneamine, —C₀₋₆alkylene-amide, —C(O)OC₁₋₄alkyl, —C₁₋₆alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl groups of any of the R⁴, R⁵, R^{5a}, R^{5b}, R⁶, R^{6a}, R^{6b}, R⁷ and R⁸ may be unsubstituted or substituted with 1, 2, 3, or 4 substituents, as allowed, independently selected from halo, —C₁₋₆alkyl, —O—C₁₋₆alkyl, —OH, or —C₁₋₆ alkyl-CN;
or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof;
as used in the present embodiment, the term “aryl” refers to a monocyclic or polycyclic aromatic group. As used in the present embodiment, aryl also refers to bicyclic and tricyclic carbon rings, where one ring is aromatic and the others are saturated, partially unsaturated, or aromatic. As used in the present embodiment, unless otherwise indicated, an aryl group can be unsubstituted or substituted with one or more, and in particular one to four, groups independently selected from, for example, halo, C₁₋₆alkyl, C₂₋₆alkenyl, C₂₋₆alkynyl, —CF₃, —OCF₃, —NO₂, —CN, —NC, —OH, alkoxy, amino, —CO₂H, —CO₂C₁₋₆alkyl, —OCOC₁₋₆ alkyl, C₃₋₁₀cycloalkyl, C₃₋₁₀heterocycloalkyl, C₅₋₁₀aryl, and C₅₋₁₀heteroaryl. As used in the present embodiment, the term “cycloalkyl” refers to a monocyclic or polycyclic non-aromatic carbocyclic ring, where the polycyclic ring can be fused, bridged, or spiro. As used in the present embodiment, the term “heterocycloalkyl” means a monocyclic or polycyclic (e.g., bicyclic), saturated or partially unsaturated, ring system containing 3 or more (e.g., 3 to 12, 4 to 10, 4 to 8, or 5 to 7) total atoms, of which one to five (e.g., 1, 2, 3, 4, or 5) of the atoms are independently selected from nitrogen, oxygen, and sulfur. As used in the present embodiment, unless otherwise indicated, a cycloalkyl or heterocycloalkyl group can be unsubstituted or substituted with one or more, and in particular one to four, groups. As used in the present embodiment, some contemplated substituents include halo, C₁₋₆ alkyl, C₂₋₆alkenyl, C₂₋₆alkynyl, —CF₃, —OCF₃, —NO₂, —CN, —NC, —OH, alkoxy, amino, —CO₂H, —CO₂C₁₋₆alkyl, —OCOC₁₋₆alkyl, C₃₋₁₀cycloalkyl, C₃₋₁₀heterocycloalkyl, C₅₋₁₀aryl, and C₅₋₁₀heteroaryl. As used in the present embodiment, the term “heteroaryl” refers to a monocyclic or polycyclic ring system (for example, bicyclic) containing one to three aromatic rings and containing one to four (e.g., 1, 2, 3, or 4) heteroatoms selected from nitrogen, oxygen, and sulfur in an aromatic ring. As used in the present embodiment, heteroaryl also refers to bicyclic and tricyclic rings, where one ring is aromatic and the others are saturated, partially unsaturated, or aromatic. As used in the present embodiment, unless otherwise indicated, a heteroaryl group can be unsubstituted or substituted with one or more, and in particular one to four or one or two, substituents. As used in the present embodiment, contemplated substituents include halo, C₁₋₆alkyl, C₂₋₆alkenyl, C₂₋₆alkynyl, —CF₃, —OCF₃, —NO₂, —CN, —NC, —OH, alkoxy, amino, —CO₂H, —CO₂C₁₋₆alkyl, —OCOC₁₋₆alkyl, C₃₋₁₀cycloalkyl, C₃₋₁₀heterocycloalkyl, C₅₋₁₀aryl, and C₅₋₁₀heteroaryl.

In embodiments, the inhibitor of KRAS G12D is a compound having a structure of the formula (CE) immediately below;

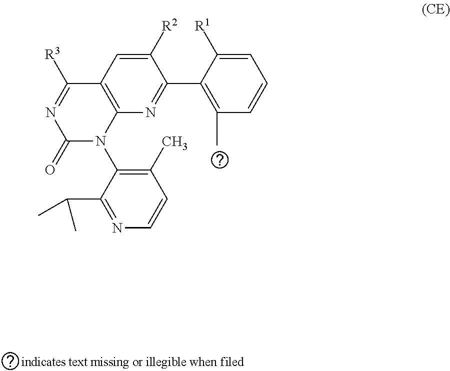

wherein
R¹ is H, halo, or —CH₃
R² is H, halo, or —CH₃;
   R³ is

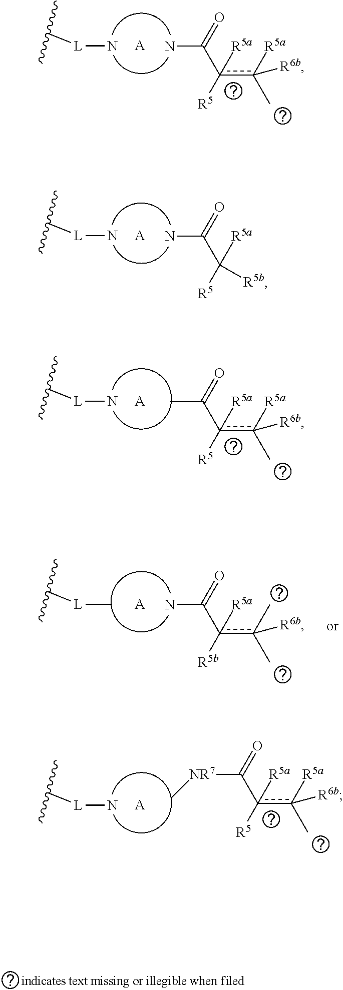

b is a single bond;
ring A is a monocyclic 4-7 membered ring or a bicyclic, bridged, fused, or spiro 6-11 membered ring;
L is a bond or NR⁴;
R⁴ is H, —C₁₋₆alkyl, —C₂₋₆alkynyl, C₁₋₆alkylene-O—C₁₋₄alkyl, C₁₋₆alkylene-OH, C₁₋₆haloalkyl, —C₁₋₆alkyleneamine, —C₀₋₆alkylene-amide, —C(O)OH, —C(O)OC₁₋₄alkyl, —C₁₋₆alkylene-O-aryl, —N═N, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R⁵ is H, halo, an —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, —C₀₋₃alkylene-C₂₋₁₄heteroaryl, or cyano;
R^{5a} is selected from H, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R^{5b} is selected from H, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
or R^{5a} and R^{5b} together, may represent an ═O or ═N═N;
R⁶ is H, halo, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆alkylene- amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R^{5a} and R^{6a}, together with the atoms to which they are attached, may form a 3-6 membered ring that optionally includes one or two heteroatoms selected from 0, S or N; or
R^{5a} and R^{6a} are absent when b is a double bond;
R^{6a} is H, or —C₁₋₆alkyl;
R^{6b} is H, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₁₋₆alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, —C₀₋₃alkylene-C₂₋₁₄heteroaryl, or cyano;
or R^{6a} and R^{6b} together, may represent an ═O;
R⁷ is H or C₁₋₈alkyl;
R⁸ is H, OH, NR^{a}R^{b};
   wherein R^{a} and R^{b} are each independently H, halo, —C₁₋₆alkyl, —C₂₋₆alkynyl;
   wherein the ring A or the —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆ alkyleneamine, —C₀₋₆alkylene-amide, —C(O)OC₁₋₄alkyl, —C₁₋₆alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl groups of any of the R⁴, R⁵, R^{5a}, R^{5b}, R⁶, R^{6a}, R^{6b}, R⁷ and R⁸ may be unsubstituted or substituted with 1, 2, 3, or 4 substituents, as allowed, independently selected from halo, —C₁₋₆alkyl, —O—C₁₋₆alkyl, —OH, or —C₁₋₆ alkyl-CN;
or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof;
as used in the present embodiment, the term “aryl” refers to a monocyclic or polycyclic aromatic group. As used in the present embodiment, aryl also refers to bicyclic and tricyclic carbon rings, where one ring is aromatic and the others are saturated, partially unsaturated, or aromatic. As used in the present embodiment, unless otherwise indicated, an aryl group can be unsubstituted or substituted with one or more, and in particular one to four, groups independently selected from, for example, halo, C₁₋₆alkyl, C₂₋₆alkenyl, C₂₋₆alkynyl, —CF₃, —OCF₃, —NO₂, —CN, —NC, —OH, alkoxy, amino, —CO₂H, —CO₂C₁₋₆alkyl, —OCOC₁₋₆ alkyl, C₃₋₁₀cycloalkyl, C₃₋₁₀heterocycloalkyl, C₅₋₁₀aryl, and C₅₋₁₀heteroaryl. As used in the present embodiment, the term “cycloalkyl” refers to a monocyclic or polycyclic non-aromatic carbocyclic ring, where the polycyclic ring can be fused, bridged, or spiro. As used in the present embodiment, the term “heterocycloalkyl” means a monocyclic or polycyclic (e.g., bicyclic), saturated or partially unsaturated, ring system containing 3 or more (e.g., 3 to 12, 4 to 10, 4 to 8, or 5 to 7) total atoms, of which one to five (e.g., 1, 2, 3, 4, or 5) of the atoms are independently selected from nitrogen, oxygen, and sulfur. As used in the present embodiment, unless otherwise indicated, a cycloalkyl or heterocycloalkyl group can be unsubstituted or substituted with one or more, and in particular one to four, groups. As used in the present embodiment, some contemplated substituents include halo, C₁₋₆alkyl, C₂₋₆alkenyl, C₂₋₆alkynyl, —CF₃, —OCF₃, —NO₂, —CN, —NC, —OH, alkoxy, amino, —CO₂H, —CO₂C₁₋₆alkyl, —OCOC₁₋₆alkyl, C₃₋₁₀cycloalkyl, C₃₋₁₀heterocycloalkyl, C₅₋₁₀aryl, and C₅₋₁₀heteroaryl. As used in the present embodiment, the term “heteroaryl” refers to a monocyclic or polycyclic ring system (for example, bicyclic) containing one to three aromatic rings and containing one to four (e.g., 1, 2, 3, or 4) heteroatoms selected from nitrogen, oxygen, and sulfur in an aromatic ring. As used in the present embodiment, heteroaryl also refers to bicyclic and tricyclic rings, where one ring is aromatic and the others are saturated, partially unsaturated, or aromatic. As used in the present embodiment, unless otherwise indicated, a heteroaryl group can be unsubstituted or substituted with one or more, and in particular one to four or one or two, substituents. As used in the present embodiment, contemplated substituents include halo, C₁₋₆alkyl, C₂₋₆alkenyl, C₂₋₆alkynyl, —CF₃, —OCF₃, —NO₂, —CN, —NC, —OH, alkoxy, amino, —CO₂H, —CO₂C₁₋₆alkyl, —OCOC₁₋₆alkyl, C₃₋₁₀cycloalkyl, C₃₋₁₀heterocycloalkyl, C₅₋₁₀aryl, and C₅₋₁₀heteroaryl.

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

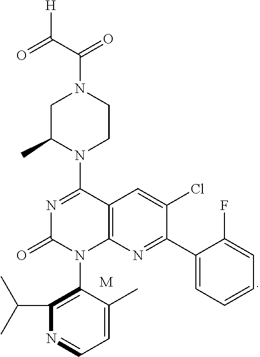

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

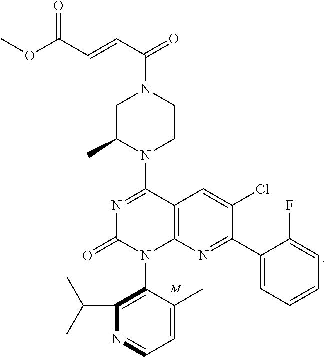

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

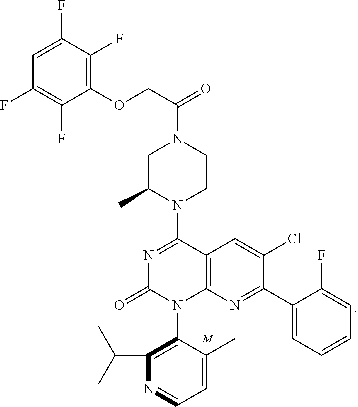

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

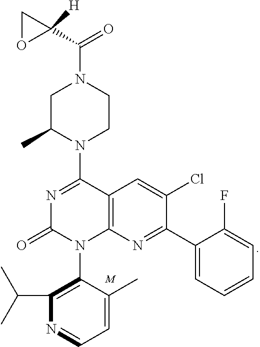

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

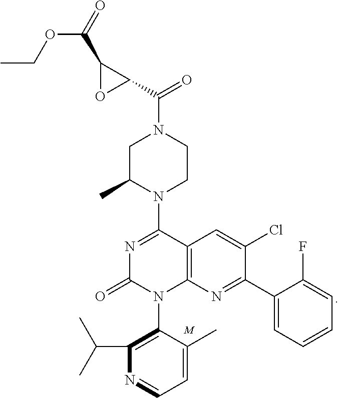

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

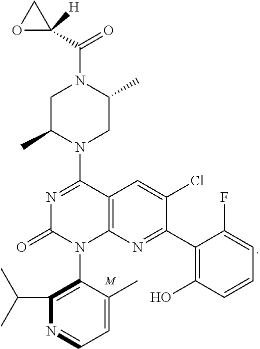

In embodiments, the inhibitor of KRAS G12D has the formula immediately below, or a stereoisomer thereof, an atropisomer thereof, a pharmaceutically acceptable salt or solvate thereof, a pharmaceutically acceptable salt of the stereoisomer thereof, or a pharmaceutically acceptable salt of the atropisomer thereof:

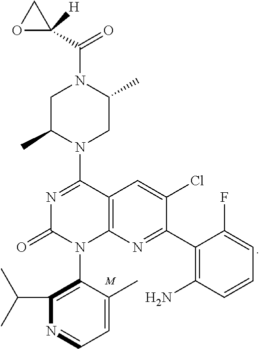

In some embodiments, the compound is an inhibitor of Ras (e.g., an inhibitor of KRas G12C, RAS, KRAS, HRAS, NRAS, KRAS G12C, KRAS G12D, HRAS G12C, or NRAS G12C). In some embodiments, the inhibitor is an inhibitor of Ras G12C (e.g., KRas G12C, mutant NRas with gly to cys mutation corresponding to position G12C of KRas (NRas G12C), mutant HRAS with gly to cys mutation corresponding to position G12C of KRas (HRas G12C)). In embodiment the inhibitor of Ras G12C is as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety.

In some embodiments, the inhibitor of KRas G12C has the structure of Formula A:

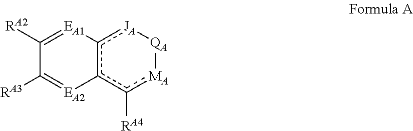

wherein:
E_{A1} and E_{A2} are each independently N or CR^{A1};
J_{A} is N, NR^{A10}, or CR^{A10};
M_{A} is N, NR^{A13}, or CR^{A13};
[symbol] is a single or double bond as necessary to give every atom its normal valence;
R^{A1} is independently H, hydroxy, C₁₋₄alkyl, C₁₋₄haloalkyl, C₁₋₄alkoxy, —NH—C₁₋₄alkyl, —N(C₁₋₄alkyl)₂, cyano, or halo;
R^{A2} is halo, C₁₋₆alkyl, C₁₋₆haloalkyl, —OR^{A′}, —N(R^{A′})₂, C₂₋₃alkenyl, C₂₋₃alkynyl, C₀₋₃alkylene-C₃₋₁₄cycloalkyl, C₀₋₃alkylene-C₂₋₁₄heterocycloalkyl, aryl, heteroaryl, C₀₋₃alkylene-C₆₋₁₄aryl, or C₀₋₃alkylene-C₂₋₁₄heteroaryl, and each R^{A′} is independently H, C₁₋₆alkyl, C₁₋₆haloalkyl, C₃₋₁₄cycloalkyl, C₂₋₁₄heterocycloalkyl, C₂₋₃alkenyl, C₂₋₃alkynyl, aryl, or heteroaryl, or two R^{A′} substituents, together with the nitrogen atom to which they are attached, form a 3-7-membered ring;
R^{A3} is halo, C₁₋₃alkyl, C₁₋₂haloalkyl, C₁₋₃alkoxy, C₃₋₄cycloalkyl, C₂₋₃alkenyl, C₂₋₃alkynyl, aryl, or heteroaryl;
R^{A4} is

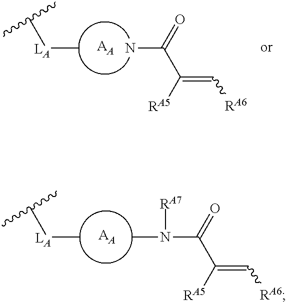

Ring A_{A} is a monocyclic 4-7 membered ring or a bicyclic, fused, or spiro 6-11 membered ring;
L_{A} is a bond, C₁₋₆alkylene, —O—C₀₋₅alkylene, —S—C₀₋₅alkylene, or —NH—C₀₋₅alkylene, and for C₂₋₆alkylene, —O—C₂₋₅alkylene, —S—C₂₋₅alkylene, and —NH—C₂₋₅alkylene, one carbon atom of the alkylene group can optionally be replaced with O, S, or NH;
R^{A5} and R^{A6} are each independently H, halo, C₁₋₆alkyl, C₂₋₆alkynyl, C₁₋₆alkylene-O—C₁₋₄alkyl, C₁₋₆alkylene-OH, C₁₋₆haloalkyl, C₁₋₆alkyleneamine, C₀₋₆alkylene-amide, C₀₋₃alkylene-C(O)OH, C₀₋₃alkylene-C(O)OC₁₋₄alkyl, C₁₋₆alkylene-O-aryl, C₀₋₃alkylene-C(O)C₁₋₄alkylene-OH, cycloalkyl, heterocycloalkyl, aryl, heteroaryl, C₀₋₃alkylene-C₃₋₁₄cycloalkyl, C₀₋₃alkylene-C₂₋₁₄heterocycloalkyl, C₀₋₃alkylene-C₆₋₁₄aryl, C₀₋₃alkylene-C₂₋₁₄heteroaryl, or cyano, or R^{A5} and R^{A6}, together with the atoms to which they are attached, form a 4-6 membered ring;
R^{A7} is H or C₁₋₈alkyl, or R^{A7} and R^{A5}, together with the atoms to which they are attached, form a 4-6 membered ring;
Q_{A} is CR^{A8}R^{A9}, C═CR^{A8}R^{A9}, C═O, C═S, or C═NR^{A8};
R^{A8} and R^{A9} are each independently H, C₁₋₃alkyl, hydroxy, C₁₋₃alkoxy, cyano, nitro, or C₃₋₆cycloalkyl, or R^{Ag} and R^{A9}, taken together with the carbon atom to which they are attached, can form a 3-6 membered ring;
R^{A10} is C₁₋₈alkyl, C₀₋₃alkylene-C₆₋₁₄aryl, C₀₋₃alkylene-C₃₋₁₄heteroaryl, C₀₋₃alkylene-C₃₋₁₄cycloalkyl, C₀₋₃alkylene-C₂₋₁₄heterocycloalkyl, C₁₋₆alkoxy, —O—C₀₋₃alkylene-C₆₋₁₄aryl, —O—C₀₋₃alkylene-C₃₋₁₄heteroaryl, —O—C₀₋₃alkylene-C₃₋₁₄cycloalkyl, —O—C₀₋₃alkylene-C₂₋₁₄heterocycloalkyl, —NH—C₁₋₈alkyl, —N(C₁₋₈alkyl)₂, —NH—C₀₋₃alkylene-C₆₋₁₄aryl, —NH—C₀₋₃alkylene-C₃₋₁₄heteroaryl, —NH—C₀₋₃alkylene-C₃₋₁₄cycloalkyl, —NH—C₀₋₃alkylene-C₂₋₁₄heterocycloalkyl, halo, cyano, or C₁₋₆ alkylene-amine; and
R^{A13} is C₁₋₆alkyl, C₁₋₆haloalkyl, C₁₋₆alkyleneamine, or C₃₋₁₄cycloalkyl.

In some embodiments, the inhibitor of KRas G12C has the structure of Formula B:

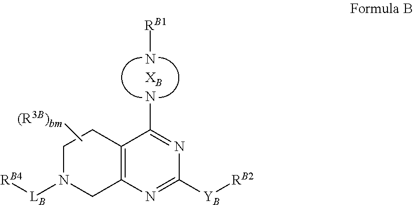

wherein:
X_{B} is a 4-12 membered saturated or partially saturated monocyclic, bridged or spirocyclic ring, wherein the saturated or partially saturated monocyclic ring is optionally substituted with one or more R^{B8};
Y_{B} is a bond, O, S, or NR^{B5};
R^{B1} is —C(O)C(R^{BA}) [symbol] C(R^{BB})_{bp} or —S(O)₂C(R^{BA}) [symbol] C(R^{BB})_{bp};
R^{B2} is hydrogen, alkyl, hydroxyalkyl, dihydroxyalkyl, alkylaminylalkyl, dialkylaminylalkyl, —Z_{B}—NR^{B5}R^{B10}, heterocyclyl, heterocyclylalkyl, aryl, heteroaryl, or heteroarylalkyl, wherein each of the Z_{B}, heterocyclyl, heterocyclylalkyl, aryl, heteroaryl, and heteroarylalkyl may be optionally substituted with one or more R^{B9};
Z_{B} is C₁-C₄ alkylene;
each R^{B3} is independently C₁-C₃ alkyl, oxo, or haloalkyl;
L_{B} is a bond, —C(O)—, or C₁-C₃ alkylene;
R^{B4} is hydrogen, cycloalkyl, heterocyclyl, aryl, aralkyl, or heteroaryl, wherein each of the cycloalkyl, heterocyclyl, aryl, aralkyl, and heteroaryl may be optionally substituted with one or more R^{B6} or R^{B7};
each R^{B5} is independently hydrogen or C₁-C₃ alkyl;
R^{B6} is cycloalkyl, heterocyclyl, heterocyclylalkyl, aryl, or heteroaryl, wherein each of the cycloalkyl, heterocyclyl, aryl, or heteroaryl may be optionally substituted with one or more R^{B7};
each R^{B7} is independently halogen, hydroxyl, C₁-C₆ alkyl, cycloalkyl, alkoxy, haloalkyl, amino, cyano, heteroalkyl, hydroxyalkyl, or -Q_{B}-haloalkyl, wherein Q_{B} is O or S;
R^{B8} is oxo, C₁-C₃ alkyl, C₂-C₄ alkynyl, heteroalkyl, cyano, —C(O)OR^{B5}, —C(O)N(R^{B5})₂, or —N(R^{B5})₂, wherein the C₁-C₃ alkyl may be optionally substituted with cyano, halogen, —OR^{B5}, —N(R^{B5})₂, or heteroaryl;
each R^{B9} is independently hydrogen, oxo, acyl, hydroxyl, hydroxyalkyl, cyano, halogen, C₁-C₆ alkyl, aralkyl, haloalkyl, heteroalkyl, cycloalkyl, heterocyclyl, heterocyclylalkyl, alkoxy, dialkylaminyl, dialkylamidoalkyl, or dialkylaminylalkyl, wherein the C₁-C₆ alkyl may be optionally substituted with cycloalkyl;
each R^{B10} is independently hydrogen, acyl, C₁-C₃ alkyl, heteroalkyl, or hydroxyalkyl;
R^{BA} is absent, hydrogen, or C₁-C₃ alkyl;
each R^{BB} is independently hydrogen, C1-C3 alkyl, alkylaminylalkyl, dialkylaminylalkyl, or heterocyclylalkyl;
bm is 0, 1, or 2; and
bp is 1 or 2;
wherein when [symbol] is a triple bond then RBA is absent, R^{BB} is present, and bp is 1,
and wherein when [symbol] is a double bond then R^{BA} is present, R^{BB} is present, and bp is 2, or R^{BA}, R^{BB} and the carbon atoms to which they are attached form a 5-8 membered partially saturated cycloalkyl optionally substituted with one or more R^{B7}.

In some embodiments, the inhibitor of Ras G12C (e.g., KRas G12C) has the structure of Formula C:

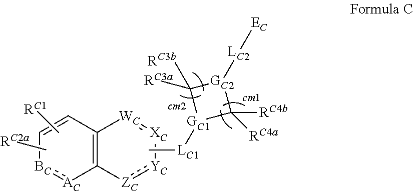

wherein:
A_{C} is CR1, CR^{C2b}, NR^{c7} or S;
B_{C} is a bond, CR^{c1} or CR^{C2};
G_{C1} and G_{C2} are each independently N or CH;
W_{C}, X_{C} and Y_{C} are each independently N, NR^{C5} or CR^{C6};
Z_{C} is a bond, N or CR^{C6}, or Z_{C} is NH when Y is C═O;
L_{C1} is a bond or NRC;
L_{C2} is a bond or alkylene;
R1 is H, cyano, halo, —CF₃, C₁-C₆alkyl, C₁-C₆alkylaminyl, C₃-C₈cycloalkyl, C₂-C₆alkenyl, or C₃-C₈cycloalkenyl, heterocyclyl, heteroaryl, aryloxy, heteroaryloxy, or aryl;
R^{C2a}, R^{C2b}, and R^{C2c} are each independently H, halo, hydroxyl, C₁-C₆alkyl, C₁-C₆haloalkyl, C₁-C₆alkoxy, C₃-C₈cycloalkyl, heteroaryl or aryl;
R^{C3a} and R^{C3b} are, at each occurrence, independently H. —OH, —NH₂, —CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl; or R^{C3a}and R^{C3b} join to form a carbocyclic or heterocyclic ring; or R^{C3a} is H, —OH, —NH₂, —CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl, and R^{C3b} joins with R^{C4b} to form a carbocyclic or heterocyclic ring;
R^{C4a} and R^{C4b} are, at each occurrence, independently H. —OH, —NH₂, CO₂H, halo, cyano, C₁-C₆alkyl, C₂-C₆alkynyl, hydroxylalkyl, aminylalkyl, alkylaminylalkyl, cyanoalkyl, carboxyalkyl, aminylcarbonylalkyl or aminylcarbonyl: or R^{C4a} and R^{C4b} join to form a carbocyclic or heterocyclic ring; or R^{C4a} is H, —OH, —NH₂, —CO₂H, halo, cyano, C₁-C₆alkyl, C₁-C₆alkynyl, hydroxylalkyl, aminylalkyl, alkylaminylalkyl, cyanoalkyl, carboxyalkyl, aminylcarbonylalkyl or aminylcarbonyl, and R^{C4b} joins with R^{C3b} to form a carbocyclic or heterocyclic ring;
R^{C5} is, at each occurrence, independently H, C₁-C₆alkyl or a bond to L_{C1};
R^{C6} is, at each occurrence, independently H, oxo, cyano, cyanoalkyl, amino, aminylalkyl, aminylalkylaminyl, aminylcarbonyl, aminylsulfonyl, —CO₂NR^{Ca}R^{Cb}, wherein R^{Ca} and R^{Cb}, are each independently H or C₁-C₆alkyl or R^{Ca} and R^{Cb} join to form a carbocyclic or heterocyclic ring, alkylaminyl, haloalkylaminyl, hydroxylalkyaminyl, amindinylalkyl, amidinylalkoxy, amindinylalkylaminyl, guanidinylalkyl, guanidinylalkoxy, guanidinylalkylaminyl, C₁-C₆alkoxy, aminylalkoxy, alkylcarbonylaminylalkoxy, C₁-C₆alkyl, heterocyclyl, heterocyclyloxy, heterocyclylalkyloxy, heterocyclylaminyl, heterocyclylalkylaminyl, heteroaryl, heteroaryloxy, heteroarylalkyloxy, heteroarylaminyl, heteroarylalkylaminyl, aryl, aryloxy, arylaminyl, arylalkylaminyl, arylalkyloxy or a bond to L_{C1};
R^{c7} is H or C₁-C₆alkyl;
cm1 and cm2 are each independently 1, 2, or 3;
[symbol] indicates a single or a double bond such that all valances are satisfied; and
E_{C} is an electrophilic moiety capable of forming a covalent bond with the cysteine residue at position 12 of a KRAS, HRAS, or NRAS G12C mutant protein;
wherein at least one of W_{C}, X_{C}, Y_{C}, and Z_{C}, is CR⁶ where R6 is a bond to L_{C1}.

In some embodiments, the inhibitor of Ras G12C (e.g., KRas G12C) has the structure of Formula D:

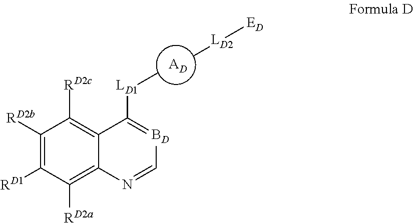

wherein:
A_{D} is a monocyclic or bicyclic moiety;
B_{D} is N or CR^{D}′;
L_{D1} is a bond or NR^{D5}; L_{D2} is a bond or alkylene;
R^{D}′ is H, cyano, alkyl, cycloalkyl, amino, aminylalkyl, alkoxy, alkoxualkyl, alkoxycarbonyl, aminylalkoxy, alkylaminylalkoxy, alkylaminyl, alkylaminylalkyl, aminylaklylaminyl, carboxyalkyl, alkylcarbonylaminyl, aminylcarbonyl, alkylaminylcarbonyl, or aminylcarbonylalkyl;
R^{D1} is aryl or heteroaryl;
R^{D2a}, R^{D2b} and R^{D2c} are each independently H, amino, halo, hydroxyl, cyano, C₁-C₆ alkyl, C₁-C₆ alkyl amino, C₁-C₆ haloalkyl (e.g., CF₃), C₁-C₆ alkoxy, C₁-C₆ haloalkoxy, C₃-C₈ cycloalkyl, heterocyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl; heteroaryl, or aryl;
R^{D5} is, at each occurrence, independently H, C₁-C₆ alkyl, C₃-C₈ cycloalkyl, or heterocyclcylalkyl; and
E_{D} is an electrophilic moiety capable of forming a covalent bond with the cysteine residue at position 12 of a KRAS, HRAS, or NRAS G12C mutant protein.

In some embodiments, the inhibitor of Ras G12C (e.g., KRas G12C) has the structure of Formula E:

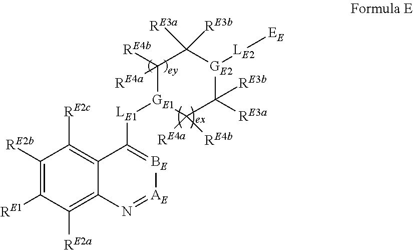

wherein:
A_{E} is N or CH;
B_{E} is N or CR^{E}′;
G^{E1} and G^{E2} are each independently N or CH;
L^{E2} is a bond or alkylene;
R^{E}′ is H, cyano, alkyl, cycloalkyl, amino, aminylalkyl, alkoxy, alkoxyalkyl, alkoxycarbonyl, aminylalkoxy, alkylaminylalkoxy, alkylaminyl, alkylaminylalkyl, aminylalkylaminyl, carboxyalkyl, alkylcarbonylaminyl, aminylcarbonyl, alkylaminylcarbonyl or aminylcarbonylalkyl;
R^{E1} is aryl or heteroaryl;
R^{E2a} and R^{E2b} are each independently amino, halo, hydroxyl, cyano, C₁-C₆ alkyl, C₁-C₆ alkyl amino, C₁-C₆ haloalkyl, C₁-C₆ alkoxy, C₁-C₆haloalkoxy, C₃-C₈ cycloalkyl, heterocycyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{E2}′ is H, amino, halo, hydroxyl, cyano, C₁-C₆ alkyl, C₁-C₆ alkyl amino, C₁-C₆ haloalkyl, C₁-C₆ alkoxy, C₁-C₆ haloalkoxy, C₃-C₈ cycloalkyl, heterocycyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{E3a} and R^{E3b} are, at each occurrence, independently H, —OH, —NH₂, —CO₂H, halo, cyano, unsubstituted C₁-C₆ alkyl, C₁-C₆ haloalkyl, C₃-C₈ cycloalkyl, heterocyclylalkyl, C₁-C₆ haloalkyl, C₁-C₆ haloalkoxy, hydroxylalkly, alkoxyalkyl, aminylalkyl, carboxyalkyl, aminylcarbonylalkyl or aminylcarbonyl; or R^{E3a} and R^{E3b} join to form oxo, a carbocyclic or heterocyclic ring; or R^{E3a} is H, —OH, —NH₂, —CO₂H, halo, cyano, C₁-C₆ alkyl, C₁-C₆ haloalkyl, C₁-C₆ haloalkoxy, C₁—C₆ alkynyl, hydroxylalkly, alkoxyalkyl, aminylalkyl, alkylaminylalkyl, cyanoalkyl, carboxyalkyl, aminylcarbonylalkyl or aminylcarbonyl, and R^{E3b} joins with R^{E4b} to form a carbocyclic or heterocyclic ring;
R^{E4a} and R^{E4b} are, at each occurrence, independently H, —OH, —NH₂, —CO₂H, halo, cyano, unsubstituted C₁-C₆ alkyl, C₁-C₆ haloalkyl, C₃-C₈ cycloalkyl, heterocyclylalkyl, C₁-C₆ haloalkyl, C₁-C₆ haloalkoxy, hydroxylalkly, alkoxyalkyl, aminylalkyl, carboxyalkyl, aminylcarbonylalkyl or aminylcarbonyl; or R^{E}a and R^{E4b} join to form oxo, a carbocyclic or heterocyclic ring; or R^{E4a} is H, —OH, —NH₂, —CO₂H, halo, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl, and R^{E4b}joins with R^{E3b} to form a carbocyclic or heterocyclic ring;
R^{E5} is, at each occurrence, independently H, C₁-C₆ alkyl, C₃-C₈cycloalkyl or heterocyclylalkyl;
ex and ey are independently integers ranging from 0 to 2; and
E_{E} is an electrophilic moiety capable of forming a covalent bond with the cysteine residue at position 12 of a KRAS, HRAS or NRAS G12C mutant protein.

In some embodiments, the inhibitor of Ras G12C (e.g., KRas G12C) has the structure of Formula F:

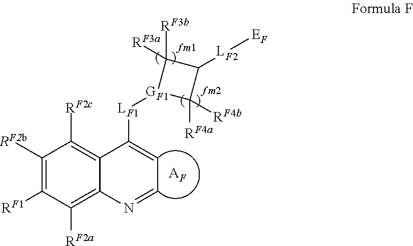

wherein:
A_{F} is a carbocyclic, heterocyclic or heteroaryl ring;
G_{F1} and G_{F2} are each independently N or CH;
L_{F1} is a bond or NR;
L_{F2} is a bond or alkylene;
R^{F1} is aryl or heteroaryl;
R^{F2a}, R^{F2b} and R^{F2c} are each independently H, amino, halo, hydroxyl, cyano, C₁-C₆ alkyl, C₁-C₆ alkyl amino, C₁-C₆haloalkyl, C₁-C₆ alkoxy, C₁-C₆ haloalkoxy; C₃-C₈ cycloalkyl, heterocyclylalkyl, C₁-C₆ alkynyl, C₁-C₆ alkenyl, aminylalkyl, alkylaminylalkyl, cyanoalkyl, carboxyalkyl, aminylcarbonylalkyl, aminylcarbonyl, heteroaryl or aryl;
R^{F3a} and R^{F3b} are, at each occurrence, independently H, —OH, —NH₂, —CO₂H, halo, cyano, C₁-C₆ alkyl, C₁-C₆haloalkyl, C₁-C₆ haloalkoxy, C₃-C₈ cycloalkyl, heterocyclylalkyl, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl; or R^{F3a} and R^{F3b} join to form a carbocyclic or heterocyclic ring; or R^{F3a} is H, OH, NH₂, CO₂H, halo, cyano, C₁-C₆ alkyl, C₁-C₆ haloalkyl, C₁-C₆ haloalkoxy, C₃-C₈ cycloalkyl, heterocyclylalkyl, C₁-C₆alkynyl, hydroxylalkly, alkoxyalkyl, aminylalkyl, alkylaminylalkyl, cyanoalkyl, carboxyalkyl, aminylcarbonylalkyl or aminylcarbonyl, and R^{F3b} joins with R^{F4b} to form a carbocyclic or heterocyclic ring;
R^{F4a} and R^{F4b} are, at each occurrence, independently H, —OH, —NH₂, —CO₂H, halo, cyano, C₁-C₆ alkyl, C₁-C₆haloalkyl, C₁-C₆ haloalkoxy, C₃-C₈ cycloalkyl, heterocyclylalkyl, C₁-C₆ alkynyl, hydroxylalkly, alkoxyalkyl, aminylalkyl, alkylaminylalkyl, cyanoalkyl, carboxyalkyl, aminylcarbonylalkyl or aminylcarbonyl; or R^{F4a} and R^{F4b} join to form a carbocyclic or heterocyclic ring; or R^{F4a} is H, —OH, —NH₂, —CO₂H, halo, cyano, C₁-C₆ alkyl, C₁-C₆ haloalkyl, C₁—C₆ haloalkoxy, C₃-C₈ cycloalkyl, heterocyclylalkyl, C₁-C₆alkynyl, hydroxylalkly, alkoxyalkyl, aminylalkyl, alkylaminylalkyl, cyanoalkyl, carboxyalkyl, aminylcarbonylalkyl or aminylcarbonyl, and R^{F4b}joins with R^{F3b} to form a carbocyclic or heterocyclic ring;
R^{F5} is, at each occurrence, independently H, C₁-C₆ alkyl, C₃-C₈ cycloalkyl or heterocycloalkyl;
fm1 and fm2 are each independently 1, 2 or 3; and
E_{F} is an electrophilic moiety capable of forming a covalent bond with the cysteine residue at position 12 of a KRAS, HRAS or NRAS G12C mutant protein.

In some embodiments, the inhibitor of KRas G12C has the structure of Formula N:

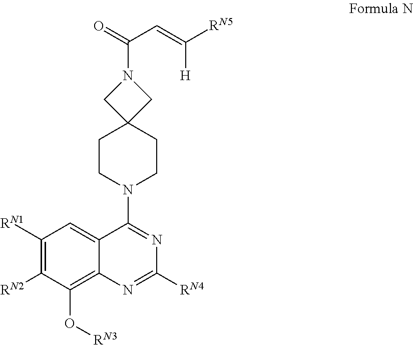

wherein:
R^{N1} is vinyl, (E)-1-propenyl or cyclopropyl;
R^{N2} is the following formula (II) or (III):

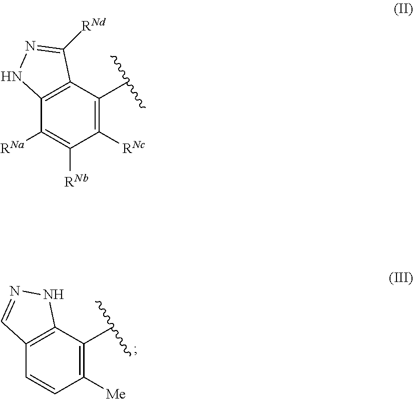

R^{N3} is C₃₋₄alkyl, methyl or n-propyl each of which may be substituted with two or more F's, ethyl or C₃₋₄cycloalkyl each of which may be substituted with F, benzyl which may be substituted with C₁₋₃alkyl, benzyl which may be substituted with —O—C₁₋₃alkyl, or benzyl which may be substituted with —O—(C₁₋₃alkyl which is substituted with F);
R^{N4} is, —O-optionally substituted C₃₋₅alkyl, —O-optionally substituted cycloalkyl, or the following formula (IV), (V), (VI) or (VII):

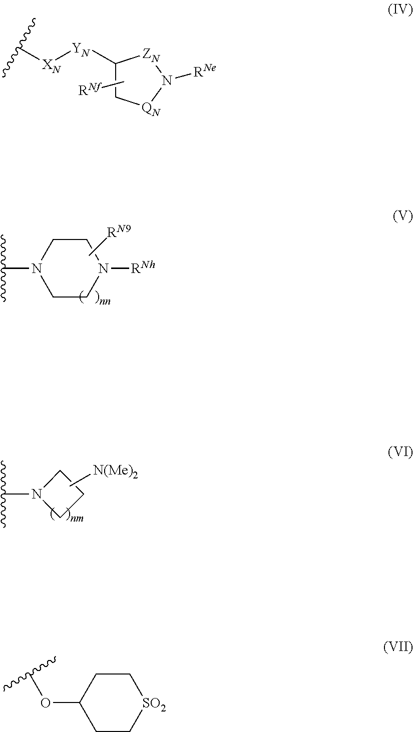

R^{N5} is H or CF₃;
R^{Na} is H or F;
R^{Nb} is H or F;
R^{Nc} is, H, methyl, vinyl or Cl;
R^{Nd} is H or Cl;
R^{Ne} is CO₂Me, COMe, CON(Me)₂, SO₂Me, C₃₋₄cycloalkyl, optionally substituted 4- to 6-membered non-aromatic heterocyclic ring, or C₁₋₃alkyl optionally substituted with a group selected from group G_{N};
Group G_{N}; —OC₁₋₃alkyl, —O—(C₁₋₃ alkyl substituted with F or C₃₋₄cycloalkyl), C₃₋₄cycloalkyl, —F, —CN, —SO₂Me, aromatic heterocyclic group, 4- to 6-membered non-aromatic heterocyclic ring, —N(C₁₋₃alkyl)₂, and —C(Me)₂OH;
R^{Nf} is, H, methyl or F;
R^{Ng} is, H, methyl or ethyl;
R^{N} is a good C 1-3 alkyl optionally substituted with —OMe;
X_{N} is, 0, NH, S or methylene;
Y_{N} is a bond or methylene;
Z_{N} is a bond, methylene or ethylene;
Q_{N} is methylene or ethylene;
nn is an integer of 1 or 2; and
nm is an integer from 1 to 3.

In some embodiments, the inhibitor of KRas G12C has the structure of Formula 0:

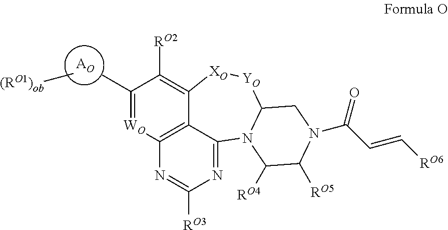

wherein:
Ring A_{O} is selected from aryl, monocyclic heteroaryl and bicyclic heteroaryl;
R^{O1} is independently selected from C₁₋₄alkyl, halo, hydroxy, C₁₋₄alkoxy, C₁₋₃fluoroalkyl, C₁₋₃fluoroalkoxy, cyano, acetylenyl, NR^{O7}R_{O8}, C(O)NR^{O9}R^{O10}, CH₂R^{O11}, N═S(O)Me₂, S(O)Me and SO₂R¹²;
ob is 0, 1, 2 or 3;
W_{O} is N or CR¹³;
X_{O} is O or NR¹⁴;
Y_{O} is CR^{O15}R^{O16}, CR^{O17}R^{O18}R^{O19}R^{O20}, C═O, or C(O)CR^{O21}R^{O22};
R^{O2} is H, cyano, halo, C₁₋₄alkyl, C₁₋₄alkoxy, C₁₋₃fluoroalkyl, NR^{O23}R^{O24}, acetylenyl or CH₂OR^{O25};
R^{O3} is H, C₁₋₃fluoroalkyl, OR^{O26}, NR^{O27}R^{O28}, CH₂R^{O29}, SR^{O30} or C(O)R^{O31};
R^{O4} is H or Me;
R^{O5} is H or Me:
R^{O6} is H or CH₂NMe₂;
R^{O7} is H, C₁₋₄alkyl, C(O)C₁₋₃alkyl or CO₂C₁₋₃alkyl;
R^{O11} is hydroxy, cyano, heterocyclyl, NR^{O12}R^{O33}, C(O)NR^{O34}R^{O35} or SO₂C₁₋₃alkyl;
R^{O12} is C₁₋₃alkyl, C₁₋₃fluoroalkyl or NR^{O36}R^{O37};
R^{O13} is H, C₁₋₄alkyl, halo, C₁₋₃fluoroalkyl or C₁₋₄alkoxy;
R^{O15}, R^{O16}, R^{O17} and R^{O18} are independently selected from H and C₁₋₃alkyl;
R^{O19}, R^{O20}, R^{O21} and R^{O22} are independently selected from H, C₁₋₃alkyl, and fluoro;
R^{O26} is selected from the group consisting of:
   H;
   C₁₋₄alkyl optionally substituted with 1 or 2 substituents selected from hydroxy, C₁₋₃ alkoxy, halo, NR^{O38}SR^{O39}, C(O)NR^{O40}R^{O41}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₁₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl; and
   heteroaryl optionally substituted with C₁₋₄alkyl, hydroxy, halo, cyano or C₁₋₄alkoxy;
R²⁷ is selected from the group consisting of:
   —H;
   C′(O)R^{O42};
   C₁₋₄alkyl optionally substituted with 1 or 2 substituents selected from hydroxy, C₁₋₃alkoxy, halo, NR^{O43}R^{O44}, C(O)NR^{O45}R^{O46}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally Further substituted with C₁₋₄alkyl, hydroxy, halo, C(O)Ne, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃fluoroalkyl, C₃₋₇cycloalkyl, CH₂cyclopropyl, heterocyclyl or heteroaryl; and
   heteroaryl optionally substituted with C₁₋₄alkyl, hydroxy, halo, cyano or C₁₋₄alkoxy;
R^{O28} is H or Me; or
R^{O27} and R^{O28} taken together with the nitrogen atom to which they are attached form a 4-, 5-, 6- or 7-membered heterocyclic ring, wherein said ring is optionally substituted with C₁₋₄alkyl, hydroxy, halo, C(O)Me, NR^{O47}R^{O48}, C₁₋₃alkoxy, C₁₋₃fluoroalkyl, C₃₋₇cycloalkyl, CH₂cyclopropyl, heterocyclyl or heteroaryl;
R^{O29} is selected from the group consisting of:
   H;
   NR^{O49}R^{O50};
   C₁₋₃alkyl optionally substituted with 1 or 2 substituents selected from hydroxy, C₁₋₃alkoxy, halo, NR^{O51}R^{O52}, C(O)NR^{O53}R^{O54}, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl; C₃₋₇cycloalkyl optionally substituted with C₁₋₄alkyl, hydroxy or halo; heterocyclyl optionally substituted with C₁₋₄alkyl, hydroxy, halo, C(O)Me, C₁₋₃alkoxy, C₁₋₃fluoroalkyl, C₃₋₇cycloalkyl, CH₂cyclopropyl, heterocyclyl or heteroaryl; and
   heteroaryl optionally substituted with C₁₋₄alkyl, hydroxy, halo, cyano or C₁₋₄alkoxy;
R^{O30} is selected from the group consisting of:
   C₁₋₄alkyl optionally substituted with 1 or 2 substituents selected from hydroxy, C₁₋₃alkoxy, halo, NR^{O55}R^{O56}, C(O)NR^{O57}R^{O58}, SO₂Me, heteroaryl, C₃₋₇cycloalkyl or heterocyclyl, wherein said heteroaryl or C₃₋₇cycloakyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
   C₃₋₇cycloalkyl optionally substituted with C₁₋₄alkyl, hydroxy or halo;
   heterocyclyl optionally substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl; and
   heteroaryl optionally substituted with C₁₋₄alkyl, hydroxy, halo, cyano or C₁₋₄alkoxy;
R^{O31} is NR^{O59}R^{O60};
R^{O42} is optionally substituted heteroaryl or optionally substituted C₁₋₄alkyl;
R^{O49} and R^{O51} are independently selected from H, C₁₋₄alkyl, heterocyclyl and heteroaryl;
R^{O59} and R^{O60} are independently selected from H and C₁₋₄alkyl; or
R^{O59} and R^{O60} taken together with the nitrogen atom to which they are attached form a 4-, 5- or 6-membered heterocyclic ring, wherein said ring is optionally substituted with C₁₋₄alkyl, hydroxy, halo or C(O)Me;
R^{O8}, R^{O9}, R^{O10}, R^{O14}, R^{O23}, R^{O24}, R^{O25}, R^{O32}, R^{O33}, R^{O34}, R^{O35}, R^{O36}, R^{O37}, R^{O38}, R^{O39}, R^{O40}, R^{O41}, R^{O43}, R^{O44}, R^{O45}, R^{O46}, R^{O47}, R^{O48}, R^{O50}, R^{O52}, R^{O53}, R^{O54}, R^{O55}, R^{O56}, R^{O57}, R^{O58}, R^{O61}, and R^{O62} are independently selected from H and C₁₋₄alkyl.

In some embodiments, the inhibitor of KRas G12C has the structure of Formula P:

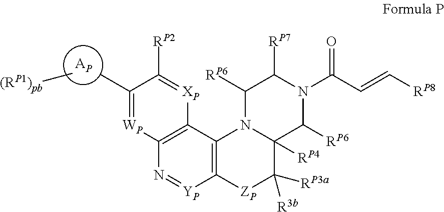

wherein:
A_{P} is selected from C₆-C₁₀ aryl, monocyclic heteroaryl and bicyclic heteroaryl;
R^{P1} is in each instance independently selected from F, Cl, Br, OH, CN, C₁-C₄ alkyl, C₁-C₄ alkoxy, C₁-C₃ fluoroalkyl, C₁-C₃ fluoroalkoxy, acetylenyl, NR^{P9}R^{P10}, C(O)NR^{P11}R^{P12}, CH₂R^{P13} and N═S(O)Me₂;
pb is 0, 1, 2 or 3;
W_{P} is CR^{P14} or N;
X_{P} is CR^{P15} or N;
Y_{P} is CH or N;
Z_{P} is 0 or NR^{P16};
R^{P2} is H, CN, F, Cl, Br, C₁-C₄ alkyl, C₁-C₄ alkoxy, C₁-C₃ fluoroalkyl, C₁-C₂ fluoroalkoxy or acetylenyl;
R^{P3a} and R^{P3b} are each independently selected from H or Me or, in the case where Z_{P} is NR¹⁶, can also together be ═O;
R^{P4}, R^{P5}, R^{P6} and R^{P7} are each independently selected from H or Me;
R^{P8} is H or CH₂NMe₂;
R^{P9} is H, C₁-C₄ alkyl, C(O)C₁-C₃ alkyl or CO₂C₁-C₃ alkyl;
R^{P10}, R^{P11} and R^{P12} are each independently selected from H and C₁-C₄ alkyl; or
R^{P9} and R^{P10} together, or R^{P11} and R^{P12} together, form a 4-, 5-, 6- or 7-membered saturated heterocycle optionally incorporating O, NH or N(C₁-C₄ alkyl) group;
R^{P13} is OH, CN, NR^{P17}R^{P18}, C(O)NR^{P19}R^{P20} or SO₂C₁-C₃alkyl;
R^{P14} and R^{P15} are each independently selected from H, F, C₁, MeO and Me;
R^{P16} is H, C₁-C₃ fluoroalkyl or CH₂R^{P21};
R^{P17}, R^{P18}, R^{P19} and R^{P20} are each independently selected from H and C₁-C₄ alkyl;
or R^{P17} and R^{P18} together, or R^{P19} and R^{P20} together, form a 4-, 5-, 6- or 7-membered saturated heterocycle optionally incorporating O, NH or N(C₁-C₄ alkyl) group;
R^{P21} is selected from the group consisting of:
   C₁-C₃ alkyl optionally substituted with 1 or 2 substituents selected from hydroxy, C₁-C₃ alkoxy, halo, NR^{P22}R^{P23}, C(O)NR^{P24}R^{P25}, SO₂Me, heteroaryl, C₃₋₇cycloalkyl or heterocyclyl, wherein said heteroaryl or C₃-C₇cycloalkyl is optionally further substituted with C₁-C₄ alkyl, hydroxy, halo, cyano, or C₁-C₄ alkoxy and said heterocyclyl is optionally further substituted with C₁-C₄ alkyl, hydroxy, halo, C(O)Me, C₁-C₃ alkoxy, C₁-C₃fluoroalkyl, C₃-C₇cycloalkyl, heterocyclyl or heteroaryl and wherein R^{P22}, R^{P23}, R^{P24} and R^{P25} are in each instance independently selected from H and C₁-C₄ alkyl;
   C₃-C₇cycloalkyl optionally substituted with C₁-C₄ alkyl, hydroxy or halo;
   heterocyclyl optionally substituted with C₁-C₄ alkyl, hydroxy, halo, C(O)Me, C₁-C₃ alkoxy, C₁-C₃ fluoroalkyl, C₃-C₇ cycloalkyl, CH₂cyclopropyl, heterocyclyl or heteroaryl; and
   heteroaryl optionally substituted with C₁-C₄ alkyl, hydroxy, halo, cyano or C₁-C₄ alkoxy.

In some embodiments, the inhibitor of KRas G12C has the structure of Formula Q:

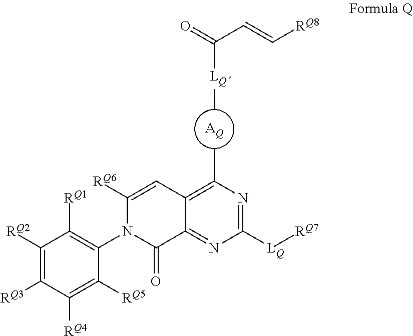

wherein:
Ring A_{Q} is 3-8 membered heterocycloalkyl, the 3-8 membered heterocycloalkyl is optionally substituted with 1, 2 or 3 of the R^{Q};
R^{Q1}, R^{Q2}, R^{Q3}, R^{Q4} and R^{Q5} are independently selected from H1, halogen, OH, NH₂, CN, C₁₋₆alkyl and C₁₋₆heteroalkyl, wherein the C₁₋₆alkyl and C₁₋₆heteroalkyl is optionally substituted with 1, 2 or 3 of the R^{Q};
or, R^{Q1} and the R^{Q2} are joined together to form ring B_{Q};
or, R^{Q2} and the R^{Q3} are joined together to form ring B_{Q};
or, R^{Q3} and the R^{Q4} are joined together to form ring B_{Q};
or, R^{Q4} and the R^{Q5} are joined together to form ring B_{Q};
Ring B_{Q} is selected from the group consisting of phenyl ring, C₅₋₆Cycloalkenyl, 5-6 membered heterocycloalkenyl and the 5-6 membered aryl, phenyl, C₅₋₆Bicycloalkenyl and 5-6 membered heterocyclenyl, 5-6 membered heteroaryl ring is optionally substituted with 1, 2 or 3 R^{Qa};
R^{Qa} is selected from halogen, OH, NH₂, CN, C₁₋₆alkyl group and C₁₋₆heteroalkyl, wherein the C₁₋₆alkyl and C₁₋₆heteroalkyl is optionally substituted with 1, 2 or 3 R^{Q};
R^{Q6} is selected from H, halogen and C₁₋₆alkyl, wherein the C₁₋₆alkyl is optionally substituted with 1, 2 or 3 of the R9; R^{Q7} is selected from the group H, CN, NH₂, C₁₋₆alkyl, C₁₋₆heteroalkyl, 4-6 membered heterocycloalkyl, 5-6 membered aryl and C₅₋₆Cycloalkyl, C₁₋₈Alkyl, C₁₋₈Heteroalkyl, 4-6 membered heterocycloalkyl, 5-6 membered aryl and C₅₋₅Cycloalkyl is optionally substituted with 1, 2 or 3 of the R^{Q};
L_{Q} is selected from single bonds, —NH—, —S—, —O—, —C(═O)—, —C(═S)—, —CH₂—, —CH(R^{Qb})— and —C(R^{Qb})₂—;
L_{Q}′ is selected from a single bond and —NH—;
R^{Qb} is selected from C₁₋₃alkyl and C₁₋₃heteroalkyl, wherein the C₁₋₃alkyl and C₁₋₃heteroalkyl is optionally substituted with 1, 2 or 3 of the R^{Q};
R^{Q8} is selected from H, C₁₋₆alkyl and C₁₋₆heteroalkyl, wherein the C₁₋₆alkyl and C₁₋₆heteroalkyl is optionally substituted with 1, 2 or 3 of the R^{Q};
R^{Q} is selected from halogen, OH, NH₂, CN, C₁₋₆alkyl, C₁₋₆heteroalkyl and C₃₋₆cycloalkyl, wherein the C₁₋₆alkyl, C₁₋₆heteroalkyl, and C₃₋₆cycloalkyl is optionally substituted with 1, 2 or 3 R^{Q}′;
R^{Q}′ is selected from F, Cl, Br, I, OH, NH₂, CN, CH₃, CH₃CH₂, CH₃O, CF₃, CHF₂, CH₂F, Cyclopropyl, propyl, isopropyl, N (CH₃)₂, NH(CH₃);
each 3-8 membered heterocyclic alkyl, C₁₋₆Heteroalkyl, 5-6 membered heterocycloalkenyl, 5-6 membered heteroaryl, C₁₋₈Heteroalkyl, 4-6 membered heterocycloalkyl, C₁₋₃Heteroalkyl contains 1, 2, or 3, “heteroatom” groups independently selected from the group of —C(═O)N(R)—, —N(R)—, —NH—, N, —O—, —S—, —C(═O)O—, —C(═O)—, —C(═S)—, —S(—O)—, —S(═O)₂— and —N(R)C(═O)N(R)—.
In some embodiments, the inhibitor of KRas G12C has the structure of Formula R:

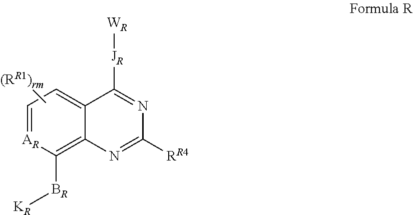

wherein:
A_{R} is —C(H)— or nitrogen;
B_{R} is oxygen, sulfur, NR^{R6} or C(R^{R6})₂;
J_{R} is a heterocycle having 3-12 ring atoms, where J_{R} is optionally substituted with 1, 2, 3, 4, 5 or 6 R^{R2};
K_{R} is C₆-C₁₂aryl, or K_{R} is heteroaryl having 5-12 ring atoms, where K_{R} is optionally substituted with 1, 2, 3, 4, 5, 6 or 7 R^{R3};
W_{R} is selected from the group consisting of:

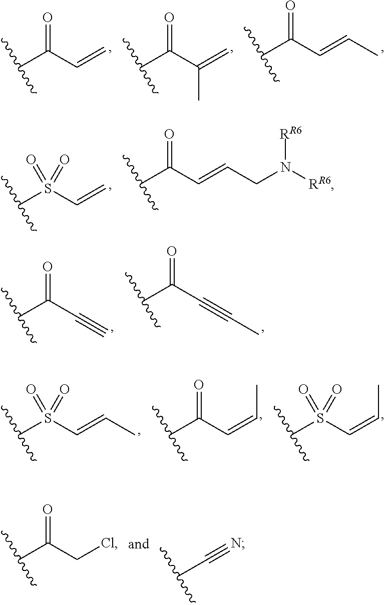

each R^{R1} is independently selected from the group consisting of C₁-C₆ alkyl, C₃-C₆ cycloalkyl, C₁-C₆ alkyl-hydroxy, C₁-C₆ alkoxy, C₁-C₆ alkyl-C₁-C₆ alkoxy, hydroxy, C₂-C₆ alkenyl, C₂-C₆ alkynyl, halogen, C₁-C₆ haloalkyl, cyano, and N(R^{R6})₂, or two R^{R1} optionally join to form a heterocycle having 3-12 ring atoms or a C₃-C₆ cycloalkyl;
each R^{R2} is independently selected from the group consisting of C₁-C₆ alkyl, C₃-C₆ cycloalkyl, hydroxy, C₁-C₆ alkyl-hydroxy, C₁-C₆ alkoxy, halogen, C₁-C₆ haloalkyl, cyano, C₁-C₆ alkylcyano, and oxo, or two R^{R2} optionally join to form a heterocycle having 3-12 ring atoms or a C₃-C₆ cycloalkyl;
each R^{R3} is independently selected from the group consisting of C₁-C₆ alkyl, C₃-C₆ cycloalkyl, hydroxy, C₁-C₆ alkoxy, halogen, C₁-C₆ halo-alkyl, —N(R^{R6})₂, oxo, and cyano, or two R^{R3} optionally join to form a heterocycle having 3-12 ring atoms or C₃-C₆ cycloalkyl;
R^{R4} is —X_{R}—Y_{R}—Z_{R} where:
   X_{R} is absent or is selected from the group consisting of oxygen, sulfur and —NR^{R6}—;
   Y_{R} is absent or C₁-C₆ alkylenyl; and
   Z_{R} is selected from H, —N(R^{R6})₂, —C(O)—N(R^{R6})₂, —OR^{R6}, heterocycle having 3-12 ring atoms, heteroaryl having 5-12 ring atoms, and C₃-C₆ cycloalkyl;
   where R^{R4} is optionally substituted with one or more R^{R7};
each R^{R5} is independently selected from the group consisting of: C₁-C₆ alkyl, hydroxy, C₁-C₆ alkoxy, halogen and —N(R^{R6})₂;
each R^{R6} is independently selected from the group consisting of hydrogen, hydroxyl, C₁-C₆ alkoxy and C₁-C₆ alkyl, or two R^{R6} optionally join to form heterocycle having 3-12 ring atoms or C₃-C₆ cycloalkyl;
each R^{R7} is independently R^{R7}′ or C₁-C₆ alkyl-R^{R7}′, where each R^{R7}′ is independently selected from the group consisting of: C₁-C₆ alkyl, hydroxy, C₁-C₆ alkoxy, halogen, —N(R^{R6})₂, heterocycle having 3-12 ring atoms, and oxo; and
rm is 0, 1, 2 or 3.

In some embodiments, the inhibitor of KRas G12C has the structure of Formula S:

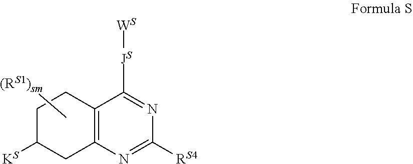

wherein:
J^{S} is a heterocycle having 3-12 ring atoms, where J⁵ is optionally substituted with 1, 2, 3, 4, 5 or 6 R^{S2};
K^{S} is C₆-C₂ aryl, or K^{S} is heteroaryl having 5-12 ring atoms, where K^{S} is optionally substituted with 1, 2, 3, 4, 5, 6 or 7 R^{S3};
W^{S} is selected from the group consisting of:

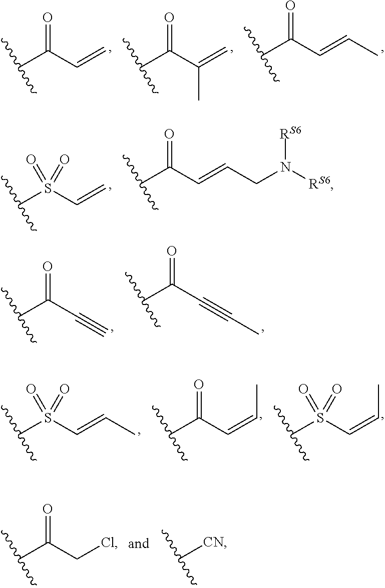

where W^{S} is optionally substituted with 1, 2 or 3 R^{S5};
each R^{S1} is independently selected from the group consisting of C₁-C₆ alkyl, C₃-C₆ cycloalkyl, C₁-C₆ alkyl-hydroxy, C₁-C₆ alkoxy, C₁-C₆ alkyl-Q-C₆alkoxy, hydroxy, C₂-C₆ alkenyl, C₂-C₆ alkynyl, halogen, C₃-C₆ haloalkyl, cyano and —N(R^{S6})₂, or two R^{S1} optionally join to form a heterocycle having 3-12 ring atoms or a C₃-C₆ cycloalkyl;
each R^{S2} is independently selected from the group consisting of C₃-C₆ alkyl, C₃-C₆ cycloalkyl, hydroxy, C₁-C₆ alkyl-hydroxy, C₁-C₆ alkoxy, halogen, C₁-C₆ haloalkyl, cyano, C₁-C₆ alkyl-cyano and oxo, or two R^{S2} optionally join to form a heterocycle having 3-12 ring atoms or a C₃-C₆ cycloalkyl;
each R^{S3} is independently selected from the group consisting of C₁-C₆ alkyl, C₃-C₆ cycloalkyl, hydroxy, C₁-C₆ alkoxy, C₁-C₆ alkyl-hydroxy, halogen, C₁-C₆ halo-alkyl, N(R^{S6})₂, oxo and cyano, or two R^{S3} optionally join to form a heterocycle having 3-12 ring atoms or C₃-C₆ cycloalkyl;
R^{S4} is —X—Y—Z where:
X^{S} is absent or is selected from the group consisting of oxygen, sulfur and —NR^{S6}—;
Y^{S} is absent or C₁-C₆ alkylenyl; and
Z^{S} is selected from H, —N(R^{S6})₂, —C(O)—N(R⁵⁶)₂, —OR^{S6}, heterocycle having 3-12 ring atoms, heteroaryl having 5-12 ring atoms, and C₃-C₆ cycloalkyl;
where R^{S4} is optionally substituted with one or more R^{S7};
each R^{S5} is independently selected from the group consisting of C₁-C₆ alkyl, hydroxy, C₁-C₆ alkoxy, halogen and —N(R^{S6})₂;
each R^{S6} is independently selected from the group consisting of hydrogen, hydroxyl, C₁-C₆ alkoxy and C₁-C₆ alkyl, or two R^{S6} optionally join to form heterocycle having 3-12 ring atoms or C₃-C₈ cycloalkyl;
each R^{S7} is independently R^{S7′} or C₁-C₆ alkyl-R^{S7′}, where each R^{S7′} is independently selected from the group consisting of C₁-C₆ alkyl, hydroxy, C₁-C₆ alkoxy, halogen, —N(R^{S6})₂, heterocycle having 3-12 ring atoms and oxo; and
sm is 0, 1, 2, 3, 4, 5, 6 or 7;

In some embodiments, the KRAS G12C inhibitor is

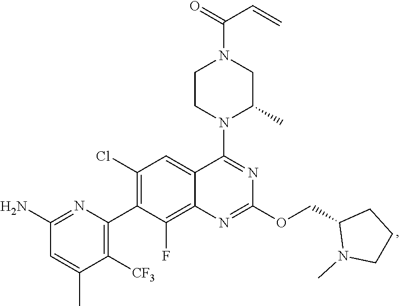 [SMILES: C=CC(=O)N1CCN(c2nc(OC[C@@H]3CCCN3C)nc3c(F)c(-c4nc(N)cc(C)c4C(F)(F)F)c(Cl)cc23)[C@@H](C)C1]

or a stereoisomer, atropisomer, solvate, or salt thereof.

In an aspect is provided a KRAS G12D inhibitor represented by the formula (CF) immediately below, or a salt thereof:

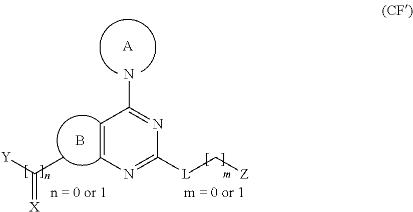

wherein Ring A represents a substituted or unsubstituted, saturated or unsaturated 8- to 10-membered N-containing bridged ring which contains at least one further heteroatom selected from the group consisting of N, S and O; Ring B represents a substituted or unsubstituted, 5- to 6-membered saturated or unsaturated ring having at least one heteroatom selected from the group consisting of N, S, and O, a 6-membered aromatic hydrocarbon ring, C3-C6 cycloalkyl ring, C3-C6 cycloalkenyl or an 8- to 10-membered spiro ring, wherein the Ring B is fused with the pyrimidine ring to form a substituted or unsubstituted bicyclic ring; n is 0 or 1; X is O or S; Y represents a substituted or unsubstituted, 6- to 10-membered unsaturated monocyclic or bicyclic ring which contains at least one heteroatom selected from the group consisting of N, S and O, or 6- to 10-membered aromatic hydrocarbon ring; L represents oxygen atom, or a substituted or unsubstituted C2-C3 alkynyl; Z represents cyanoalkyl, alkylcarbonylaminoalkyl, alkylaminocarbonyl, alkylaminoalkyl, a substituted or unsubstituted, C3-C6 cycloalkyl, a 5- to 6-membered saturated ring which contains at least one heteroatom selected from the group consisting of N, S and O, or an 8- to 10-membered partially unsaturated ring which contains at least one heteroatom selected from the group consisting of N, S and O; when L is C2-C3 alkynyl, Z is alkylaminocarbonyl or alkylaminoalkyl; m is 0 or 1. In embodiments, the Ring A is represented by the formula (3a) or (3b):

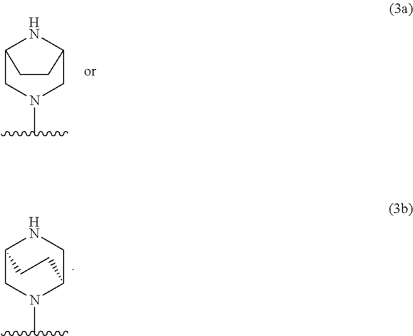 [SMILES: *N1CC2CCC(C1)N2 ; *N1C[C@@H]2CC[C@H]1CN2]

In embodiments, Z represents cyclobutane, cyclopropane, piperidine, morpholine, piperazine, isoindoline, or 1,2,3,4-tetrahydroisoquinoline which may be substituted by halogen atom, hydroxyl, C1-C3 alkoxy, methyl, ethyl, isopropanyl, ethylcalbonylmethyl, hydroxyethyl, dimethylamino, dimethylaminomethyl, methoxyethyl, cyanomethyl, morpholylmethyl, or 3-fluoropyrrolidinylmethyl. In embodiments, the KRAS G12D inhibitor or salt thereof is a compound selected from compounds (1) to (37) described immediately below: (1) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-bromonaphbthalen-2-ol, (2) 4-(4-(3,8-diazabicyclo[3.2.1]octan-3-yl)-2-((1-(((R)-3-fluoropyrrolidin-1-yl)methyl)cyclopropyl)methoxy)-5,8-dihydropyrido[3,4-d]pyrimidin-7(6H)-yl)-5-bromonaphthalen-2-ol, (3) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-bromonaphthalen-2-ol, (4) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-y)-5-methylnaphthalen-2-ol, (5) 4-(4-(3,8-diazabicyclo[3.2.1]octan-3-yl)-2-((1-{((R})-3-fluoropyrrolidin-1-yl)methyl)cyclopropyl)methoxy)-5,8-dihydropyrido[3,4-d]pyrimidin-7(61H)-yl)-5-methylnaphthalen-2-ol, (6) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-methylnaphthalen-2-ol, (7) 4-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-5-iodonaphthalen-2-ol, (8) 4-(4-(3,8-diazabicyclo[3.2.1]octan-3-yl)-2-((1-(((R-3-fluoropyrrolidin-1-yl)methyl)cyclopropyl)methoxy)-5,8-dihydropyrido[3,4-d]pyrimidin-7(6H)-yl)-5-iodonaphthalen-2-ol, (9) 4-(4-(3,8-diazabicyclo[3.2.1]octan-3-yl)-2-((1-(morpholinomethyl)cyclopropyl)methoxy)-5,8-dihydropyrido[3,4-d]pyrimidin-7(6H)-yl)-5-iodonaphthalen-2-ol, (10) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl)(8-ethynyl-3-hydroxynaphthalen-1-yl)methanone, (11) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl)(8-ethynyl-3-hydroxynaphthalen-1-yl)methanone, (12) (4-(3,8-diazabicyclo[3.2.1]octan-3-yl)-2-((1-(((R)-3-fluoropyrrolidin-1-yl)ethyl)cyclopropyl)methoxy)-5,7-dihydro-6H-pyrrolo[3,4-d]pyrimidin-6-yl)(3-hydroxy-8-iodonaphthalen-1-yl)methanone, (13) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl)(3-hydroxy-8-iodonaphthalen-1-yl)methanone, (14) 4-(4-(3,8-diazabicyclo[3.2.1]octan-3-yl)-6-chloro-2-((1-((dimethylamino)methyl)cyclopropyl)methoxy)-8-fluoroquinazolin-7-yl)naphthalen-2-ol, (15) 4-(4-(3,8-diazabicyclo[3.2.1]octan-3-yl)-2-((1-((dimethylamino)methyl)cyclopropyl)methoxy)-8-fluoroquinazolin-7-y)naphthalen-2-ol, (16) 4-(4-(3,8-diazabicyclo[3.2.1]octan-3-yl)-8-fluoro-2-(((S)-1-methylpyrrolidin-2-yl)methoxy)quinazolin-7-yl)naphthalen-2-ol, (17) 4-(4-(3,8-diazabicyclo[3.2.1]octan-3-yl)-2-((cis-2-(dimethylamino)cyclobutyl)methoxy)-8-fluoroquinazolin-7-yl)naphthalen-2-ol, (18) 4-(4-(3,8-diazabicyclo[3.2.1]octan-3-yl)-2-((1-((dimethylamino)methyl)cyclopropyl)methoxy)-6,8-difluoroquinazolin-7-yl)naphthalen-2-ol (19) 4-(4-(3,8-diazabicyclo[3.2.1]octan-3-yl)-2-((1-((dimethylamino)methyl)cyclopropyl)methoxy)-6-ethyl-8-fluoroquinazolin-7-yl)naphthalen-2-ol, (20) 4-(4-((1S,4S)-2,5-diazabicyclo[2.2.2]octan-2-yl)-2-((1-((dimethylamino)methyl)cyclopropyl)methoxy)-5,8-dihydropyrido[3,4-d]pyrimidin-7(6H)-yl)-5-bromonaphthalen-2-ol, (21) 4-(4-(3,8-diazabicyclo[3.2.1]octan-3-yl)-2-((1-((dimethylamino)methyl)-2,2-difluorocyclopropyl)methoxy)-5,8-dihydropyrido[3,4-d]pyrimidin-7(6)-yl)-5-bromonaphthalen-2-ol, (22) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-bromoisoquinolin-3-amine, (23) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-ethynylnaphthalen-2-ol, (24) 1-(1-(((4-(3,8-diazabicyclo[3.2.1]octan-3-yl)-7-(8-ethynylnaphthalen-1-yl)-5,6,7,8-tetrahydroquinazolin-2-yl)oxy)methyl)cyclopropyl)-N,N-dimethylmethanamine, (25) 4-((1-(((4-(3,8-diazabicyclo[3.2.1]octan-3-yl)-7-(8-iodonaphthalen-1-yl)-5,6,7,8-tetrahydropyrido[3,4-d]pyrimidin-2-yl)oxy)methyl)cyclopropyl)methyl)morpholine, (26) 4-(4-(3,8-diazabicyclo[3.2.1]octan-3-yl)-2-(((R)-1-methylpyrrolidin-2-yl)methoxy)-5,8-dihydropyrido[3,4-d]pyrimidin-7(6H)-yl)-5-bromonaphthalen-2-ol, (27) 4-(4-(3,8-diazabicyclo[3.2.1]octan-3-yl)-2-(((2S,4R)-4-fluoro-1-methylpyrrolidin-2-yl)methoxy)-5,8-dihydropyrido[3,4-d]pyrimidin-7(6-H)-yl)-5-bromonaphthalen-2-ol, (28) 4-(4-((1S,4S)-2,5-diazabicyclo[2.2.2]octan-2-yl)-2-((1-(morpholinomethyl)cyclopropyl)methoxy)-5,8-dihydropyrido[3,4-d]pyrimidin-7(61H1)-yl)-5-bromonaphthalen-2-ol, (29) 1-(1-(((4-(3,8-diazabicyclo[3.2.1]octan-3-yl)-7-(8-bromonaphthalen-1-yl)-5,6,7,8-tetrahydropyrido[3,4-d]pyrimidin-2-yl)oxy)methyl)-2,2-difluorocyclopropyl)-N,N-dimethylmethanamine, (30) 4-(4-(3,8-diazabicyclo[3.2.1]octan-3-yl)-2-((1-((dimethylamino)methyl)-2,2-dimethylcyclopropyl)methoxy)-5,8-dihydropyrido[3,4-d]pyrimidin-7(6H)-yl)-5-bromonaphthalen-2-ol, (31) 4-(4-(3,8-diazabicyclo[3.2.1]octan-3-yl)-2-(((S)-1-methylpyrrolidin-2-yl)methoxy)-5,8-dihydropyrido[3,4-d]pyrimidin-7(6H)-yl)-5-bromonaphthalen-2-ol, (32) 4-(4-(3,8-diazabicyclo[3.2.1]octan-3-yl)-2-(((2S,4R)-4-methoxy-1-methylpyrrolidin-2-yl)methoxy)-5,8-dihydropyrido[3,4-d]pyrimidin-7(6H)-yl)-5-bromonaphthalen-2-ol, (33) 4-(4-(3,8-diazabicyclo[3.2.1]octan-3-yl)-2-((1- (morpholinomethyl)cyclopropyl)methoxy)-7,8-dihydro-5H-pyrano[4,3-d]pyrimidin-7-yl)-5-ethynylnaphthalen-2-ol, (34) (4-(3,8-diazabicyclo[3.2.1]octan-3-yl)-2-((1-(((R)-3-fluoropyrrolidin-1-yl)methyl)cyclopropyl)methoxy)-5,7-dihydro-6H-pyrrolo[3,4-d]pyrimidin-6-yl)(3-hydroxy-8-vinylnaphthalen-1-yl)methanone, (35) (4-(3,8-diazabicyclo[3.2.1]octan-3-yl)-2-((1-(morpholinomethyl)cyclopropyl)methoxy)-5,7-dihydro-6H-pyrrolo[3,4-d]pyrimidin-6-yl)(3-hydroxy-8-vinylnaphthalen-1-yl)methanone, (36) 1-(4-(3,8-diazabicyclo[3.2.1]octan-3-yl)-2-((1-((dimethylamino)methyl)cyclopropyl)methoxy)-5,8-dihydropyrido[3,4-d]pyrimidin-7(6H)-yl)-8-ethynylisoquinolin-3-amine, and (37) 4-(4-(3,8-diazabicyclo[3.2.1]octan-3-yl)-2-((1-((dimethylamino)methyl)cyclopropyl)methoxy)-7,8-dihydro-5H-pyrano[4,3-d]pyrimidin-7-yl)-5-bromonaphthalen-2-ol.

In an aspect, the present disclosure features KRAS G12D inhibitors of Formula (CG′) immediately below: A-L-B (Formula CG′) wherein A is a Ras binding moiety; L is a linker; and B is a selective cross-linking group, or a pharmaceutically acceptable salt thereof. Embodiments described in the present paragraph are applicable only to aspects and embodiments of the present paragraph. In some embodiments, upon contacting the KRAS G12D inhibitor, or a pharmaceutically acceptable salt thereof, with sample containing a Ras protein, at least 20% of the Ras protein in the sample covalently reacts with the KRAS G12D inhibitor, or a pharmaceutically acceptable salt thereof, to form a conjugate. In some embodiments, upon contacting the KRAS G12D inhibitor, or a pharmaceutically acceptable salt thereof, with a sample containing a Ras protein, at least 20% (e.g., at least 30%, at least 40%, at least 50%, at least 60%, at least 70%, at least 80%, at least 90%, at least 95%, at least 99%) of the Ras protein in the sample covalently reacts (e.g., forms a conjugate including the Ras binding moiety, the linker, and the Ras protein) with the KRAS G12D inhibitor, or a pharmaceutically acceptable salt thereof, to form a conjugate. In some embodiments, the Ras binding moiety is a human H-Ras binding moiety, a human N-Ras binding moiety, or a human K-Ras binding moiety. In some embodiments, the Ras binding moiety is a K-Ras binding moiety. In some embodiments, the K-Ras binding moiety binds to a residue of a K-Ras Switch-II binding pocket of the K-Ras protein, e.g., a residue of the K-Ras protein corresponding to V7, V8, V9, G10, A11, D12, K16, P34, T58, A59, G60, Q61, E62, E63, Y64, S65, R68, D69, Y71, M72, F78, 192, H95, Y96, Q99, I100, R102, or V103 of human wild-type K-Ras. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. In some embodiments, the Ras binding moiety comprises the structure of any one of Formula II to V, described immediately below. In some embodiments, the Ras binding moiety (e.g., K-Ras binding moiety) includes the structure of Formula II:

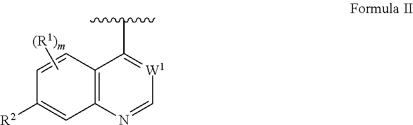

wherein m is 0, 1, 2, or 3; Wi is N or C, wherein C is optionally attached to the linker via an optionally substituted C₁-C₃ alkylene bridge or optionally substituted C₁-C₃ heteroalkylene bridge; each R¹ is, independently, CN, halo, hydroxy, optionally substituted C₁-C₆ alkyl, or optionally substituted C₁-C₆ heteroalkyl, or R¹ is attached to the linker via a C₁-C₃ alkylene bridge or C₁-C₃ heteroalkylene bridge; and R² is optionally substituted C₆-C₁₀ aryl or optionally substituted C₂-C₉ heteroaryl. In some embodiments of Formula II, W¹ is N or C, wherein C is attached to the linker via an optionally substituted C₁-C₃ alkylene bridge or optionally substituted C₁-C₃ heteroalkylene bridge. In some embodiments, the Ras binding moiety (e.g., K-Ras binding moiety) includes the structure:

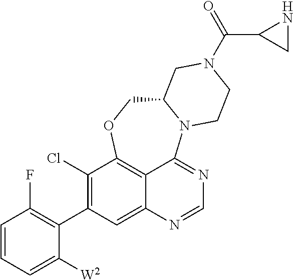

wherein W² is hydrogen or hydroxy. In some embodiments, the Ras binding moiety (e.g., K-Ras binding moiety) includes the structure of Formula III:

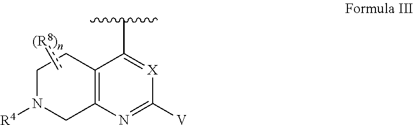

wherein n is 0, 1, 2, 3, 4, 5, or 6; [symbol] represents a single bond or a double bond; X is N or CR′, wherein R′ is hydrogen, or R′ is attached to the linker via an optionally substituted C₁-C₃ alkylene bridge, or optionally substituted C₁-C₃ heteroalkylene bridge; V is CHR⁵, CR⁵R⁶, OR⁵, NHR⁵, or NR^{5a}R^{5b}; each R³ is, independently, oxo, optionally substituted C₁-C₆ alkyl, or optionally substituted C₁-C₆ heteroalkyl, or R³ is attached to the linker via an optionally substituted C₁-C₃ alkylene bridge or optionally substituted C₁-C₃ heteroalkylene bridge; R⁴ is optionally substituted C₆-C₁₀ aryl or optionally substituted C₂-C₉ heteroaryl; each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl, or R^{5a} and R^{5b}, together with the nitrogen atom to which each is attached, combine to form optionally substituted C₂-C₉ heterocyclyl; provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and further provided that when R³ is attached to the linker via an optionally substituted C₁-C₃ alkylene bridge or optionally substituted C₁-C₃ heteroalkylene bridge, R′ is not attached to the linker via an optionally substituted C₁-C₃ alkylene bridge or optionally substituted C₁-C₃ heteroalkylene bridge. In some embodiments, the Ras binding moiety (e.g., K-Ras binding moiety) includes the structure of Formula IV immediately below:

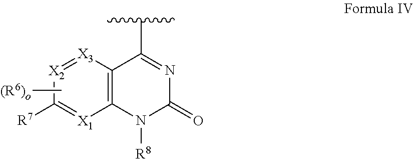

wherein o is 0, 1, or 2; X¹, X² and X³ are each independently N, CH, or CR⁶; each R⁶ is, independently, halo, CN, hydroxy, optionally substituted C₁-C₆ alkyl, or optionally substituted C₁-C₆ heteroalkyl, or R⁶ is attached to the linker via a C₁-C₃ alkyl bridge or C₁-C₃ heteroalkyl bridge; and R⁷ and R⁸ are, independently, optionally substituted C₆-C₁₀ aryl or optionally substituted C₂-C₉ heteroaryl, or a pharmaceutically acceptable salt thereof. In some embodiments, the Ras binding moiety (e.g., K-Ras binding moiety) includes the structure of Formula V immediately below:

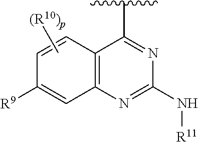

Formula V wherein p is 0, 1, 2, or 3; R⁹ is optionally substituted C₆-C₁₀ aryl or optionally substituted C₂-C₉ heteroaryl; each R¹⁰ is, independently, halo, CN, hydroxy, optionally substituted C₁-C₆ alkyl, or optionally substituted C₁-C₆ heteroalkyl, or R¹⁰ is attached to the linker via a C₁-C₃ alkylene or C₁-C₃ heteroalkylene bridge; and R¹¹ is optionally substituted C₂-C₉ heteroaryl or optionally substituted C₂-C₉ heterocyclyl, or a pharmaceutically acceptable salt thereof. In some embodiments, the Ras binding moiety includes the structure of a Ras moiety described in WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223, the Ras binding moieties of which are herein incorporated by reference. In view of the disclosures herein as well as general knowledge, persons of skill in the art will understand how a cross-linking group of a KRAS G12D inhibitor of these references may be replaced with a selective cross-linking group of the present invention. KRAS G12D inhibitors, or a pharmaceutically acceptable salt thereof, of the present invention include a linker between a Ras binding moiety (e.g., A, in Formula CG′) and a selective cross-linking group (e.g., B, in Formula CG′). As used herein, a “linker” as used in Formula CG′ above refers to a divalent organic moiety connecting moiety A to moiety B in a KRAS G12D inhibitor of Formula CG′, such that the resulting KRAS G12D inhibitor is capable of achieving an IC50 of 2 μM or less in the Ras-RAF disruption assay protocol provided in Lim et al., Angew. Chem. Int. Ed. 53:199 (2014). In some embodiments, a linker has the structure of Formula VI immediately below: -A¹-(B¹)a-(C¹)b-(B²)c-(D)-(B³)d-(C²)e-(B⁴)f-A²- Formula VI where A¹ is a bond between the linker and the Ras binding moiety; A² is a bond between the selective cross-linking group and the linker; B¹, B², B³, and B⁴ each, independently, is selected from optionally substituted C₁-C₂ alkylene, optionally substituted C₁-C₃ heteroalkylene, 0, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇heteroalkyl; C¹ and C² are each, independently, selected from carbonyl, thiocarbonyl, sulphonyl, or phosphoryl; a, b, c, d, e, and f are each, independently, 0 or 1; and D is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₂₋₆ heteroarylene, optionally substituted C₃₋₈ cycloalkylene, optionally substituted C₆₋₁₂ arylene, optionally substituted C₂-C₁₀ polyethylene glycol, or optionally substituted C₁₋₁₀ heteroalkylene, or a chemical bond linking A¹-(B¹)a-(C¹)b-(B²)c- to —(B³)d-(C²)e-(B⁴)f-A². In some embodiments, the linker is an optionally substituted heterocyclyl group, such as an optionally substituted 3 to 8-membered heterocyclyl group. In some embodiments, the linker is an optionally substituted cycloalkyl group, such as an optionally substituted 3 to 8-membered carbocyclyl group. In some embodiments, the linker is as exemplified in any of Formulas VIIa to VIIb.

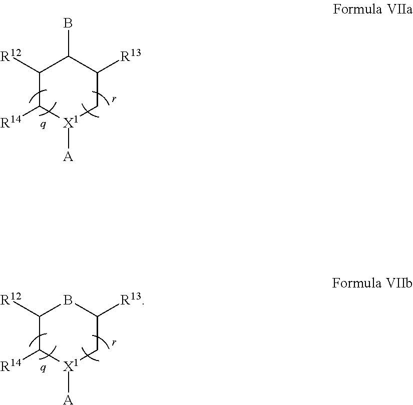

In these structures, when a nitrogen group is at position B, that nitrogen is part of the selective cross-linking group. When a carbon atom is at position B, that carbon atom is part of the linker. In some embodiments, the KRAS G12D inhibitor A-L-B, or a pharmaceutically acceptable salt thereof, has the structure of any one of Formula VIIao or VIIbo:

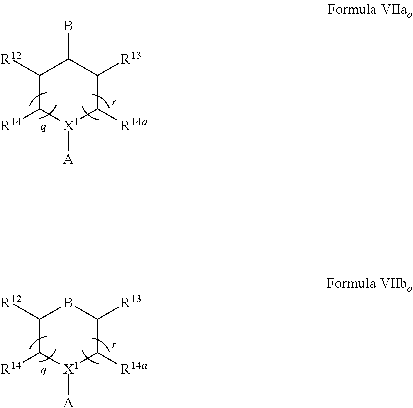

wherein q and r are, independently, 0, 1, or 2; X¹ is N or CH; and R¹², R¹³, R¹⁴ and R^{14a} are, independently, hydrogen, oxo, optionally substituted C₁-C₆ alkyl, optionally substituted C₁-C₆ heteroalkyl, or —CO₂-optionally substituted C₁-C₆ alkyl, wherein when R¹⁴ is not oxo, R¹⁴ optionally comprises a bond to A. In some embodiments, R¹², R¹³, R¹⁴ and R^{14a} are not simultaneously oxo. In some embodiments, only one of R¹², R¹³, R¹⁴ and R^{14a} is oxo. In some embodiments, A-L-B, or a pharmaceutically acceptable salt thereof, is selected from the group consisting of:

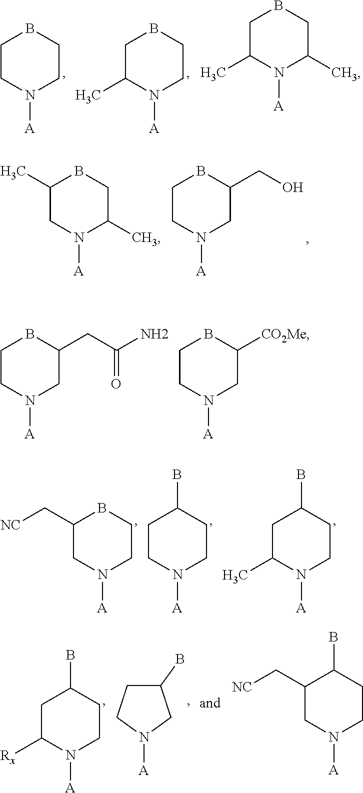

wherein RX is an optionally substituted C₁-C₃ alkylene bridge or optionally substituted C₁-C₃ heteroalkylene bridge joined to A (see, e.g., WO 2018/206539). In some embodiments, A-L-B, or a pharmaceutically acceptable salt thereof, is

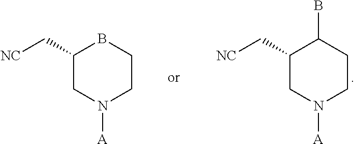

In some embodiments, -L-B is selected from the group consisting of:

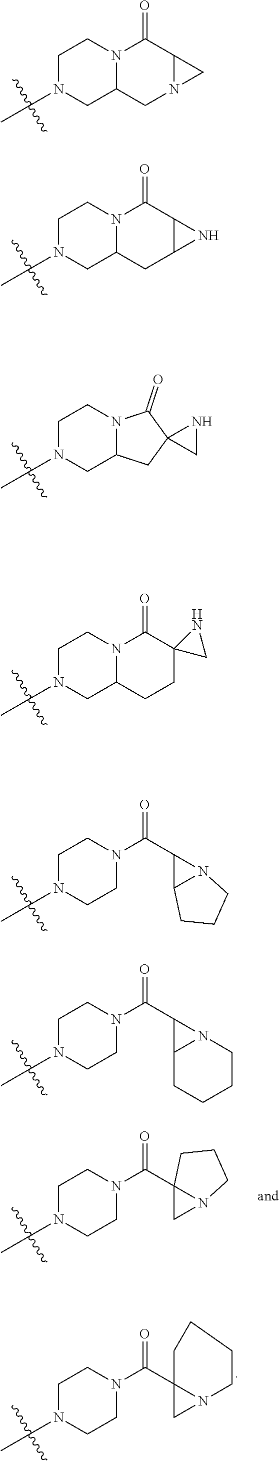

In some embodiments, A-L-B, or a pharmaceutically acceptable salt thereof, is the structure of Formula VIIc or Formula VIId:

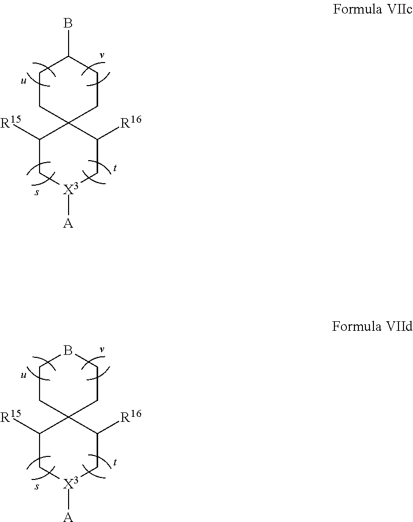

wherein s, t, u, and v are, independently, 0, 1, or 2; X³ is N or CH; and R¹⁵ and R¹⁶ are, independently, hydrogen, optionally substituted C₁-C₆ alkyl or optionally substituted C₁-C₆ heteroalkyl. See also Formula VIIf, below, for a depiction of the linker moiety in these formulas. In some embodiments, A-L-B, or a pharmaceutically acceptable salt thereof, is:

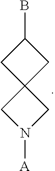

In some embodiments, the linker is acyclic. For example, the linker is the structure of Formula VIII:

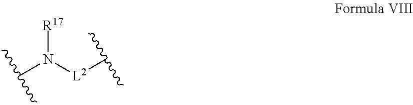

wherein R¹⁷ is hydrogen or optionally substituted C₁-C₆ alkyl; and L² is optionally substituted C₁-C₁₄alkylene or optionally substituted C₃-C₆ cycloalkyl. KRAS G12D inhibitors, or a pharmaceutically acceptable salt thereof, as described herein include a selective cross-linking group. As used herein, “selective cross-linking group” refers to a group which exhibits cross-linking reactivity preferentially with one or more Ras protein nucleophilic functional groups in comparison to other nucleophilic functional groups that exist in a Ras protein, under conventional conditions of organic synthesis or under physiological conditions. For example, in some embodiments, a selective cross-linking group reacts preferentially with a carboxyl group, a hydroxy group, or a thiol group, or a combination thereof, in comparison with other nucleophilic functional groups in a Ras protein. For example, in some embodiments, a selective cross-linking group reacts preferentially with a carboxyl group. In some embodiments, a selective cross-linking group reacts preferentially with a hydroxy group. In some embodiments, a selective cross-linking group reacts preferentially with a thiol group. In some embodiments, a selective cross-linking group reacts preferentially with a carboxyl group and a hydroxy group. In some embodiments, a selective cross-linking group reacts preferentially with a carboxyl group and a thiol group.

In some embodiments, a selective cross-linking group reacts preferentially with a hydroxy group and a thiol group. Non-limiting examples of moieties which are “selective cross-linking groups” include, for example, a carbodiimide, an aminooxazoline, a chloroethyl urea, an aziridine, a trifluoromethyl ketone, a boronic acid, a boronic ester, N-ethoxycarbonyl-2-ethoxy-1,2-dihydroquinoline (EEDQ), an iso-EEDQ or other EEDQ derivative, an epoxide, an oxazolium, or a glycal. In some embodiments, a selective cross-linking group is a carbodiimide, an aminooxazoline, a chloroethyl urea, an aziridine, a trifluoromethyl ketone, a boronic acid, a boronic ester, an epoxide, or a glycal. In some embodiments, a selective cross-linking group is a carbodiimide, an aminooxazoline, a chloroethyl urea, or an aziridine. In some embodiments, the selective cross-linking group is a C—O bond forming selective cross-linking group. In some embodiments, the selective cross-linking group is a C—S bond forming selective cross-linking group. In some embodiments, the selective cross-linking group has the structure or is comprised within any one of Formula IX to XVIII immediately below. In some embodiments, the selective cross-linking group is the structure of Formula IX:

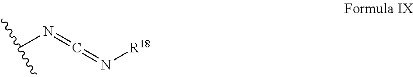

wherein R¹⁸ is optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl. In some embodiments, the selective cross-linking group is the structure of Formula Xa or Xb:

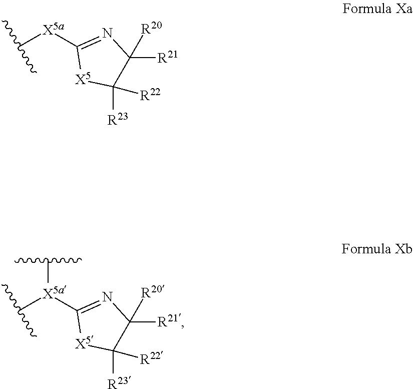

respectively, wherein X⁵ is O or S; X^{5′} is O or S; X^{5a} is absent or NR¹⁹; X^{5a′} is N, wherein said N is a ring atom of an optionally substituted C₂-C₉ heterocyclyl group; R¹⁹ is hydrogen, optionally substituted C₁-C₆ alkyl, optionally substituted C₆-C₁₀ aryl, optionally substituted C₂-C₉ heterocyclyl, or optionally substituted C₂-C₉ heteroaryl; and R²⁰, R²¹, R²², R²³, R^{20′}, R^{21′}, R^{22′}, and R^{23′} are, independently, hydrogen or optionally substituted C₁-C₆ alkyl. In some embodiments, the selective cross-linking group is the structure of Formula XIa or XIb:

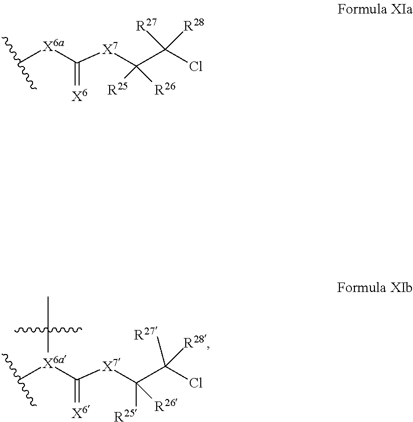

respectively, wherein X⁶ is O or S; X^{6′} is O or S; is a sent or NR²⁴; X^{6a′} is N, wherein said N is a ring atom of an optionally substituted C₂-C₉ heterocyclyl group; X⁷ and X^{7′} are each O, S, or NR²⁹; R²⁴ is hydrogen, optionally substituted C₁-C₆ alkyl, optionally substituted C₆-C₁₀ aryl, optionally substituted C₂-C₉ heterocyclyl, or optionally substituted C₂-C₉ heteroaryl; and R²⁵, R²⁶, R²⁷, R²⁸, R²⁹, R^{25′}, R^{26′}, R^{27′}, and R^{28′} are, independently, hydrogen or optionally substituted C₁-C₆ alkyl. In some embodiments, the selective cross-linking group is the structure of Formula XIIa, XIIb, XIIc, XIId or XIIe immediately below:

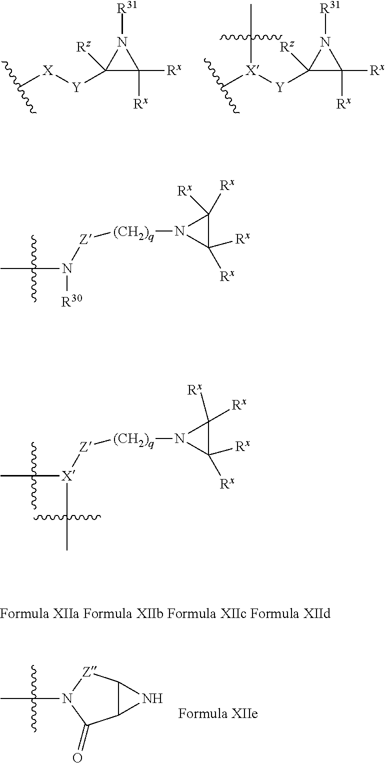

wherein X is absent or NR³⁰; X′ is N, wherein said N is a ring atom of an optionally substituted C₂-C₉ heterocyclyl group; Y is C(O), C(S) (that is, C═S), SO₂, or optionally substituted C₁-C₃ alkyl; Z′ is C(O) or SO₂; Z″ is —CH₂— or C(O); q is 0, 1, or 2; each R^{X} is, independently, hydrogen, CN, C(O)R^{Y}, CO₂Ry, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl; each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl; R^{Z} is hydrogen or CH₃; R³⁰ is hydrogen or optionally substituted C₁-C₆ alkyl; R³¹ is hydrogen, —C(O)R³², —SO₂R³³, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and R³² and R³³ are, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl. In some embodiments, the KRAS G12D inhibitor has a structure selected from

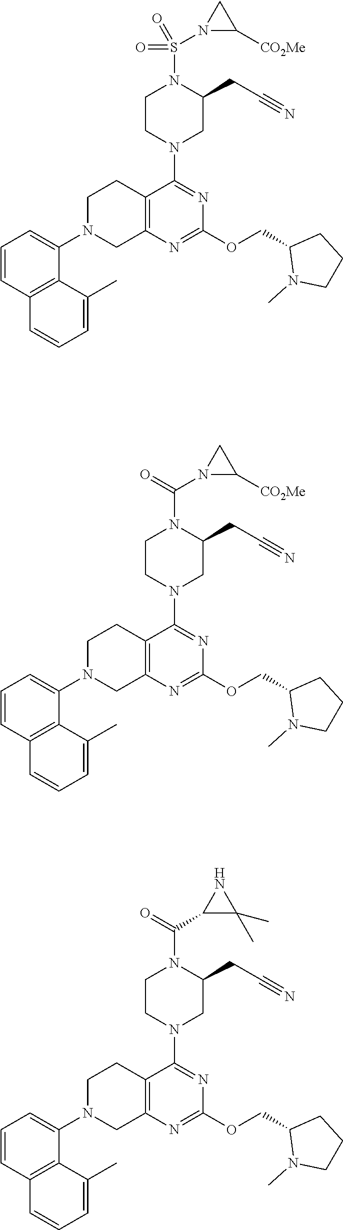 [SMILES: COC(=O)C1CN1S(=O)(=O)N1CCN(c2nc(OC[C@@H]3CCCN3C)nc3c2CCN(c2cccc4cccc(C)c24)C3)C[C@@H]1CC#N ; COC(=O)C1CN1C(=O)N1CCN(c2nc(OC[C@@H]3CCCN3C)nc3c2CCN(c2cccc4cccc(C)c24)C3)C[C@@H]1CC#N ; Cc1cccc2cccc(N3CCc4c(nc(OC[C@@H]5CCCN5C)nc4N4CCN(C(=O)[C@@H]5NC5(C)C)[C@@H](CC#N)C4)C3)c12]

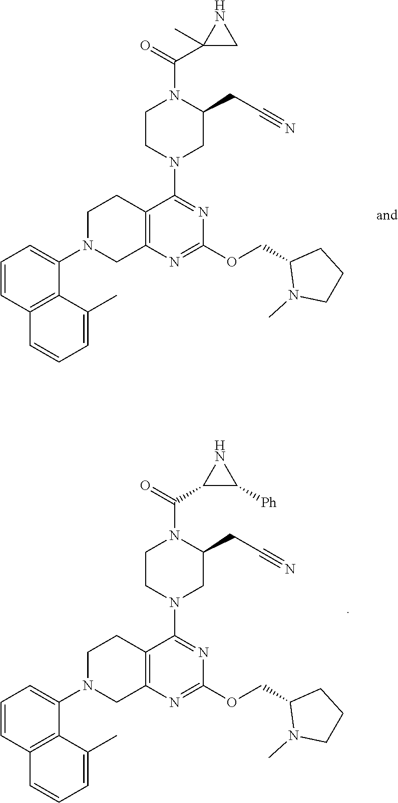 [SMILES: Cc1cccc2cccc(N3CCc4c(nc(OC[C@@H]5CCCN5C)nc4N4CCN(C(=O)C5(C)CN5)[C@@H](CC#N)C4)C3)c12 ; Cc1cccc2cccc(N3CCc4c(nc(OC[C@@H]5CCCN5C)nc4N4CCN(C(=O)[C@@H]5N[C@@H]5c5ccccc5)[C@@H](CC#N)C4)C3)c12]

In some embodiments, the KRAS G12D inhibitor has the structure:

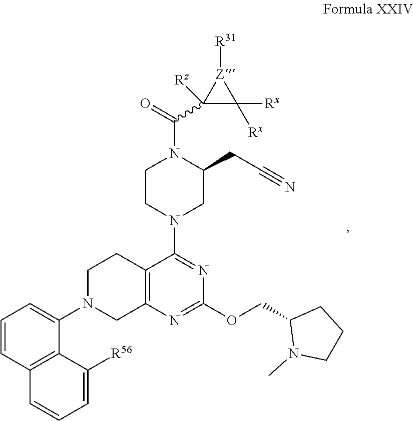

wherein R31 is absent, hydrogen, C(O)CH₃, SO₂CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl; R⁵⁶ is CH₃ or C₁; R^{Z} is hydrogen, optionally substituted C₁-C₃ alkyl; each R^{x} is, independently, hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and Z′″ is N or O. In some embodiments, the KRAS G12D inhibitor has the structure:

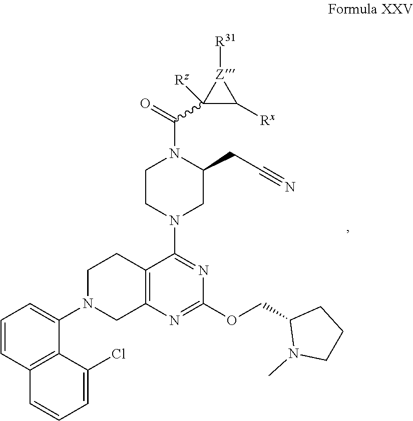

wherein R³¹ is absent, hydrogen, C(O)CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl; R^{Z} is hydrogen, optionally substituted C₁-C₃ alkyl; R^{x} is hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and Z′″ is N or O. In some embodiments, the selective cross-linking is:

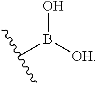 [SMILES: *B(O)O]

In some embodiments, the selective cross-linking group is the structure of Formula XIV:

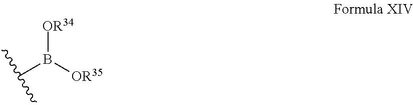

wherein R³⁴ and R³⁵ are, independently, optionally substituted C₁-C₆ alkyl, or R³⁴ and R³⁵ combine with the boron to which they are attached to form an optionally substituted heterocyclyl. In some embodiments, the selective cross-linking group is the structure of Formula XV:

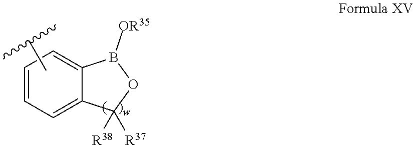

wherein w is 1 or 2; R³⁶ is hydrogen or optionally substituted C₁-C₆ alkyl; and each R³⁷ and R³⁸ is, independently, hydrogen or optionally substituted C₁-C₆ alkyl. In some embodiments, the selective cross-linking group is the structure of Formula XVI:

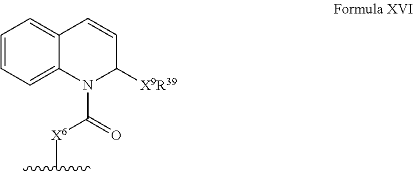

wherein X⁸ is absent, O, S, NR⁴⁰, or CH₂; X⁹ is O, NR⁴¹, S, S(O), or S(O)₂; R³⁹ is optionally substituted C₁-C₆ alkyl; and R⁴⁰ and R⁴¹ are, independently, hydrogen or optionally substituted C₁-C₆ alkyl. In some embodiments, the selective cross-linking group is the structure of Formula XVII:

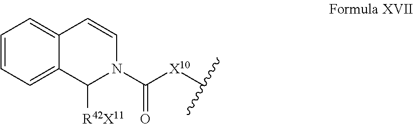

wherein X¹⁰ is absent, O, S, NR⁴³, or CH₂; X¹¹ is O, NR⁴⁴, S, S(O), or S(O)₂; R⁴² is optionally substituted C₁-C₆ alkyl; and R⁴³ and R⁴⁴ are, independently, hydrogen or optionally substituted C₁-C₆ alkyl. In some embodiments, the selective cross-linking group is the structure of Formula XVIII im:

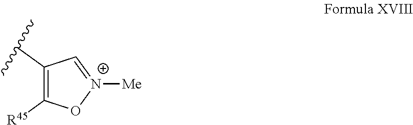

wherein R⁴⁵ is hydrogen or optionally substituted C₁-C₆ alkyl. In some embodiments, the selective cross-linking group is the structure of Formula XIX:

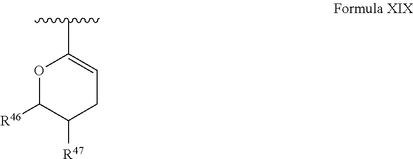

wherein R⁴⁶ and R⁴⁷ are, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl. In some embodiments, a KRAS G12D inhibitor of the present invention has the structure of Formula XX or XXI:

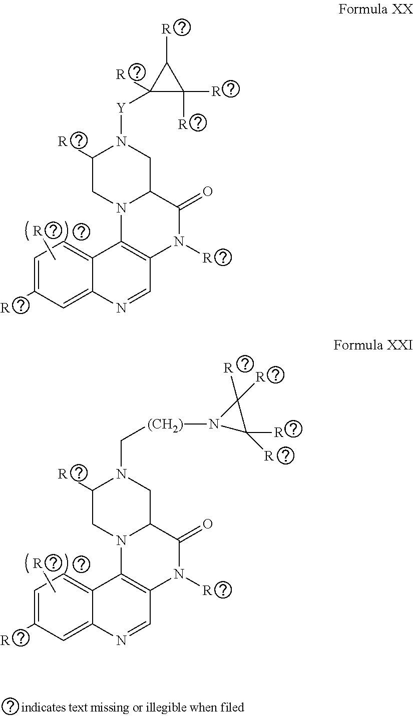

wherein Y is C(O), C(S), SO₂, or optionally substituted C₁-C₆ alkyl; Z′ is C(O) or SO₂; q is 0, 1 or 2; x is 0, 1, 2 or 3; each R^{X} is, independently, hydrogen, CN, C(O)R^{y}, CO₂R^{y}, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl; each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl; each R⁴⁸ is, independently, CN, halo, hydroxy, optionally substituted C₁-C₆ alkyl, or optionally substituted C₁-C₆ heteroalkyl, or R⁴⁹ is optionally substituted C₆-C₁₀ aryl or optionally substituted C₂-C₉ heteroaryl; R⁵⁰ is hydrogen or C₁-C₆ alkyl; R^{S1} is hydrogen, CN or C₁-C₆ alkyl; R⁵⁴ is hydrogen, —C(O)R³², —SO₂R₃₃, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and R⁵⁵ is hydrogen or optionally substituted C₁-C₆ alkyl. The term “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain KRAS G12D inhibitors of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein. Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocyclyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR₂; —N(R^{∘})C(S)NR₂; —(CH₂)₀₋₄N(R^{∘})C(O)OR^{∘}; —N(R^{∘})N(R^{∘})C(O)R^{∘}; —N(R^{∘})N(R^{∘})C(O)NR₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR₂; —(CH₂)₀₋₄C(O)SR^{∘}; —(CH₂)₀₋₄C(O)OSiR₃; —(CH₂)₀₋₄ OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O)NR₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR₂; —N(R^{∘})S(O)₂R^{∘}; —N(OR^{∘})R^{∘}; —C(NOR °)NR₂; —C(NH)NR₂; —P(O)₂R^{∘}; —P(O)R₂; —P(O)(OR^{∘})₂; —OP(O)R₂; —OP(O) (OR^{∘})₂; —OP(O)(OR^{∘})R^{∘}, —SiR₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R °, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below. Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S. Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R)S(O)₂R^{†}; wherein each Rit is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S. The term “aryl,” as used in this paragraph, represents a monovalent mono-, bicyclic, or multicyclic ring system formed by carbon atoms, wherein each ring is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified. The term “cycloalkyl,” as used herein, represents a monovalent saturated cyclic hydrocarbon group, which may be bridged, fused or spirocyclic, which may be fused, having from three to eight ring carbons, unless otherwise specified, and is exemplified by cyclopropyl, cyclobutyl, cyclopentyl, cyclohexyl, cycloheptyl, cycloheptyl, and the like. The term “heteroalkyl” as used in this paragraph refers to an “alkyl” group, as defined herein, in which at least one carbon atom has been replaced with a heteroatom (e.g., an O, N, or S atom). The heteroatom may appear in the middle or at the end of the radical. The term “heteroaryl,” as used in this paragraph, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups as used in this paragraph are of 1 to 12 (e.g., 1 to 11, 1 to 10, 1 to 9, 2 to 12, 2 to 11, 2 to 10, or 2 to 9) carbons. The term “heteroaryl” as used in this paragraph includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups as used in this paragraph include, but are not limited to, pyridyl, pyrazolyl, benzooxazolyl, benzoimidazolyl, benzothiazolyl, imidazolyl, thiazolyl, quinolinyl, isoquinolinyl, tetrahydroquinolinyl, 4-azaindolyl, or and the like. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups. The term “heterocyclyl,” as used in this paragraph, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” as used in this paragraph also represents a heterocyclic compound having a bridged multicyclic structure in which one or more carbons or heteroatoms bridges two non-adjacent members of a monocyclic ring, e.g., a quinuclidinyl group. The term “heterocyclyl” as used in this paragraph includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocyclyl groups as used in this paragraph are pyrrolidinyl, piperidinyl, 1,2,3,4-tetrahydroquinolinyl, decahydroquinolinyl, dihydropyrrolopyridine, decahydronapthyridinyl, or and the like. A heterocyclic ring can be attached to its pendant group at any ring atom that results in a stable structure and any of the ring atoms can be optionally substituted unless otherwise specified. It will be understood that when an aspect or embodiment (e.g., a method described herein) refers to a compound of a formula (e.g., Formula CG′), embodiments of the compound of the formula (e.g., Formula CG′) include any of the formulae that are sub-genera or species of the formula (e.g, described in the present paragraph as well as Formula CG′ itself).

In an aspect is provided a KRAS G12D, or pharmaceutically acceptable salt thereof, of structural Formula CH′:

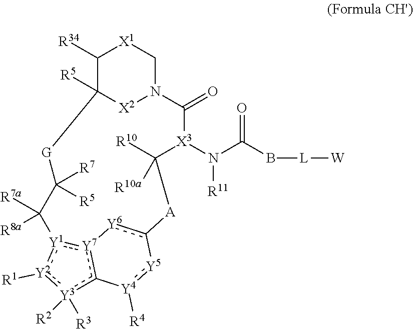

wherein the dotted lines represent zero, one, two, three, or four non-adjacent double bonds; A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene; B is —CH(R⁹)— or >C═CR⁹R₉′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene; G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene; L is absent or a linker; W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal; X¹ is optionally substituted C₁-C₂ alkylene, NR, O, or S(O)n; X² is O or NH; X³ is N or CH; n is 0, 1, or 2; R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl; Y¹ is C, CH, or N; Y², Y³, Y⁴, and Y⁷ are, independently, C or N; Y⁵ is CH, CH₂, or N; Y⁶ is C(O), CH, CH₂, or N; R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or R¹ and R² combine with the atoms to which they are attached to form an optionally substituted 3 to 14-membered heterocycloalkyl; R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or R² and R³ combine with the atom to which they are attached to form an optionally substituted 3 to 8-membered cycloalkyl or optionally substituted 3 to 14-membered heterocycloalkyl; R⁴ is absent, hydrogen, halogen, cyano, or methyl optionally substituted with 1 to 3 halogens; R⁵ is hydrogen, C₁-C₄ alkyl optionally substituted with halogen, cyano, hydroxy, or C₁-C₄ alkoxy, cyclopropyl, or cyclobutyl; R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3 to 6-membered cycloalkyl or optionally substituted 3 to 7-membered heterocycloalkyl; R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or R⁷ and R⁸ combine with the carbon atom to which they are attached to form C═CR⁷′R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl; R^{7a} and R^{8a} are, independently, hydrogen, halo, optionally substituted C₁-C₃ alkyl, or combine with the carbon to which they are attached to form a carbonyl; R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or R^{7′} and R^{8′} combine with the carbon atom to which they are attached to form optionally substituted 3 to 6-membered cycloalkyl or optionally substituted 3 to 7-membered heterocycloalkyl; R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or R⁹ and L combine with the atoms to which they are attached to form an optionally substituted 3 to 14-membered heterocycloalkyl; R^{9′} is hydrogen or optionally substituted C₁-C₆ alkyl; R¹⁰ is hydrogen, halo, hydroxy, C₁-C₃ alkoxy, or C₁-C₃ alkyl; R^{10a} is hydrogen or halo; R¹¹ is hydrogen or C₁-C₃ alkyl; and R³⁴ is hydrogen or C₁-C₃ alkyl (e.g., methyl). The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein. Suitable monovalent substituents on a substitutable carbon atom of an “optionally substituted” group may be, independently, deuterium; halogen; —(CH₂)₀₋₄R\hat{ }; —(CH₂)₀₋₄OR\hat{ }; —O(CH₂)₀₋₄R^{∘}; —O—(CH₂)₀₋₄C(O)OR^{∘}; —(CH₂)₀₋₄CH(OR\hat{ })₂; —(CH₂)₀₋₄SR\hat{ }; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4 to 8-membered saturated or unsaturated heterocycloalkyl (e.g., pyridyl); 3 to 8-membered saturated or unsaturated cycloalkyl (e.g., cyclopropyl, cyclobutyl, or cyclopentyl); —NO₂; —CN; —N₃; —(CH₂)₀₋₄N(R\hat{ })₂; —(CH₂)₀₋₄N(R\hat{ })C(O)R\hat{ }; —N(R\hat{ })C(S)R\hat{ }; —(CH₂)₀₋₄N(R\hat{ })C(O)NR\hat{ }₂; —N(R\hat{ })C(S)NR\hat{ }₂; —(CH₂)₀₋₄N(R\hat{ })C(O)OR\hat{ }; —N(R\hat{ })N(R\hat{ })C(O)R\hat{ }; —N(R\hat{ })N(R\hat{ })C(O)NR\hat{ }₂; —N(R\hat{ })N(R\hat{ })C(O)OR\hat{ }; —(CH₂)₀₋₄C(O)R\hat{ }; —C(S)R\hat{ }; —(CH₂)₀₋₄C(O)OR\hat{ }; —(CH₂)₀₋₄—C(O)—N(R^{∘})₂; —(CH₂)₀₋₄—C(O)—N(R^{∘})—S(O)₂—R^{∘}; —C(NCN)NR—(CH₂)₀₋₄C(O)SR\hat{ }; —(CH₂)₀₋₄C(O)OSi R \hat{ }₃; —(CH₂)₀₋₄OC(O)R\hat{ }; —OC(O)(CH₂)₀₋₄SR\hat{ }; —SC(S)SR^{∘}; —(CH₂)₀₋₄SC(O)R\hat{ }; —(CH₂)₀₋₄C(O)NR\hat{ }₂; —C(S)NR\hat{ }₂; —C(S)SR^{∘}; —(CH₂)₀₋₄OC(O)NR\hat{ }₂; —C(O)N(OR\hat{ })R\hat{ }; —C(O)C(O)R\hat{ }; —C(O)CH₂C(O)R\hat{ }; —C(NOR\hat{ })R\hat{ }; —(CH₂)₀₋₄SSR\hat{ }; —(CH₂)₀₋₄S(O)₂R\hat{ }; —(CH₂)₀₋₄S(O)₂OR\hat{ }; —(CH₂)₀₋₄OS(O)₂R\hat{ }; —S(O)₂NR\hat{ }₂; —(CH₂)₀₋₄S(O)R\hat{ }; —N(R\hat{ })S(O)₂NR\hat{ }₂; —N(R\hat{ })S(O)₂R\hat{ }; —N(OR\hat{ })R\hat{ }; —C(NOR\hat{ })NR \hat{ }₂; —C(NH)NR\hat{ }₂; —P(O)₂R\hat{ }; —P(O)R\hat{ }₂; —P(O)(OR\hat{ })₂; —OP(O)R\hat{ }₂; —OP(O)(OR\hat{ })₂; —OP(O)(OR\hat{ })R\hat{ }, —SiR\hat{ }₃; —(C₁-C₄ straight or branched alkylene)O—N(R\hat{ })₂; or —(C₁-C₄ straight or branched alkylene)C(O)O—N(R\hat{ })₂, wherein each R\hat{ } may be substituted as defined below and is independently hydrogen, —C₁-C₆ aliphatic, —CH₂Ph, —O(CH₂)₀₋₁Ph, —CH₂-(5 to 6 membered heteroaryl ring), or a 3 to 6-membered saturated, partially unsaturated, or aryl ring having 0-4 heteroatoms independently selected from nitrogen, oxygen, or sulfur, or, notwithstanding the definition above, two independent occurrences of R\hat{ }, taken together with their intervening atom(s), form a 3 to 12-membered saturated, partially unsaturated, or aryl mono- or bicyclic ring having 0-4 heteroatoms independently selected from nitrogen, oxygen, or sulfur, which may be substituted as defined below. Suitable monovalent substituents on R\hat{ } (or the ring formed by taking two independent occurrences of R\hat{ } together with their intervening atoms), may be, independently, halogen, —(CH₂)₀₋₂R\hat{ }, -(haloR\hat{ }), —(CH₂)₀₋₂OH, —(CH₂)₀₋₂OR\hat{ }, —(CH₂)₀₋₂CH(OR\hat{ })₂; —O(haloR\hat{ }), —CN, —N₃, —(CH₂)₀₋₂C(O)R\hat{ }, —(CH₂)₀₋₂C(O)OH, —(CH₂)₀₋₂C(O)OR\hat{ }, —(CH₂)₀₋₂SR\hat{ }, —(CH₂)₀₋₂SH, —(CH₂)₀₋₂NH₂, —(CH₂)₀₋₂NHR\hat{ }, —(CH₂)₀₋₂NR \hat{ }₂, —NO₂, -SiR\hat{ }₃, —OSiR\hat{ }₃, —C(O)SR\hat{ }, —(C₁₋₄ straight or branched alkylene)C(O)OR\hat{ }, or —SSR\hat{ } wherein each R\hat{ } is unsubstituted or where preceded by “halo” is substituted only with one or more halogens, and is independently selected from C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R\hat{ } include ═O and ═S. Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁-C₆ aliphatic which may be substituted as defined below, or an unsubstituted 5 to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁-C₆ aliphatic which may be substituted as defined below, or an unsubstituted 5 to 6-membered saturated, partially unsaturated, or aryl ring having 0-4 heteroatoms independently selected from nitrogen, oxygen, or sulfur. Suitable substituents on the aliphatic group of R* include halogen, —R\hat{ }, -(haloR\hat{ }), —OH, —OR\hat{ }, —O(haloR\hat{ }), —CN, —C(O)OH, —C(O)OR\hat{ }, —NH₂, —NHR\hat{ }, —NR\hat{ }₂, or —NO₂, wherein each R is unsubstituted or where preceded by “halo” is substituted only with one or more halogens, and is independently C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur. Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁-C₆ aliphatic which may be substituted as defined below, unsubstituted —OPh, or an unsubstituted 3 to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3 to 12-membered saturated, partially unsaturated, or aryl mono- or bicyclic ring having 0-4 heteroatoms independently selected from nitrogen, oxygen, or sulfur. Suitable substituents on an aliphatic group of R^{†} are independently halogen, —R\hat{ }, -(haloR\hat{ }), —OH, —OR\hat{ }, —O(haloR\hat{ }), —CN, —C(O)OH, —C(O)OR\hat{ }, —NH₂, —NHR\hat{ }, —NR\hat{ }₂, or —NO₂, wherein each R\hat{ } is unsubstituted or where preceded by “halo” is substituted only with one or more halogens, and is independently C₁-C₄ aliphatic, —CH₂Ph, —O(CH₂)₀₋₁Ph, or a 5 to 6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S. The term “aryl,” as used in this paragraph, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified. The term “cycloalkyl,” as used in this paragraph, represents a monovalent saturated cyclic hydrocarbon group, which may be bridged, fused, or spirocyclic having from three to eight ring carbons, unless otherwise specified, and is exemplified by cyclopropyl, cyclobutyl, cyclopentyl, cyclohexyl, cycloheptyl, and cycloheptyl. The term “heteroalkyl,” as used in this paragraph, refers to an “alkyl” group, as defined herein, in which at least one carbon atom has been replaced with a heteroatom (e.g., an O, N, or S atom). The heteroatom may appear in the middle or at the end of the radical. The term “heteroaryl,” as used in this paragraph, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” as used in this paragraph includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups as used in this paragraph include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups. The term “heterocycloalkyl,” as used in this paragraph, represents a monovalent, monocyclic, bicyclic or polycyclic ring system, which may be bridged, fused, or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” as used in this paragraph also represents a heterocyclic compound having a bridged multicyclic structure in which one or more carbons or heteroatoms bridges two non-adjacent members of a monocyclic ring, e.g., a quinuclidinyl group. The term “heterocycloalkyl” as used in this paragraph includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. In embodiments, R¹ is

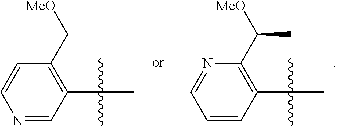 [SMILES: *c1cnccc1COC ; *c1cccnc1[C@H](C)OC]

In some embodiment, the linker is the structure of Formula It: A¹-(B¹)f-(C¹)g-(B²)h-(D¹)-(B³)i-(C²)j-(B⁴)k-A² Formula II where A is a bond between the linker and B; A² is a bond between W and the linker; B′, B², B³, and B4 each, independently, is selected from optionally substituted C₁-C₂ alkylene, optionally substituted C₁-C₃ heteroalkylene, 0, S, and NR^{N}; RN is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)f-(C¹)g-(B²)h- to —(B³)i-(C²)j-(B⁴)k-A². In some embodiments, the linker is acyclic. In some embodiments, the linker has the structure of Formula IIa:

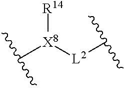

Formula IIa wherein Xa is absent or N; R¹⁴ is absent, hydrogen or optionally substituted C₁-C₆ alkyl; and L² is absent, —SO₂—, optionally substituted C₁-C₄ alkylene or optionally substituted C₁-C₄ heteroalkylene, wherein at least one of X^{a}, R⁴, or L² is present. In some embodiments, the linker has the structure:

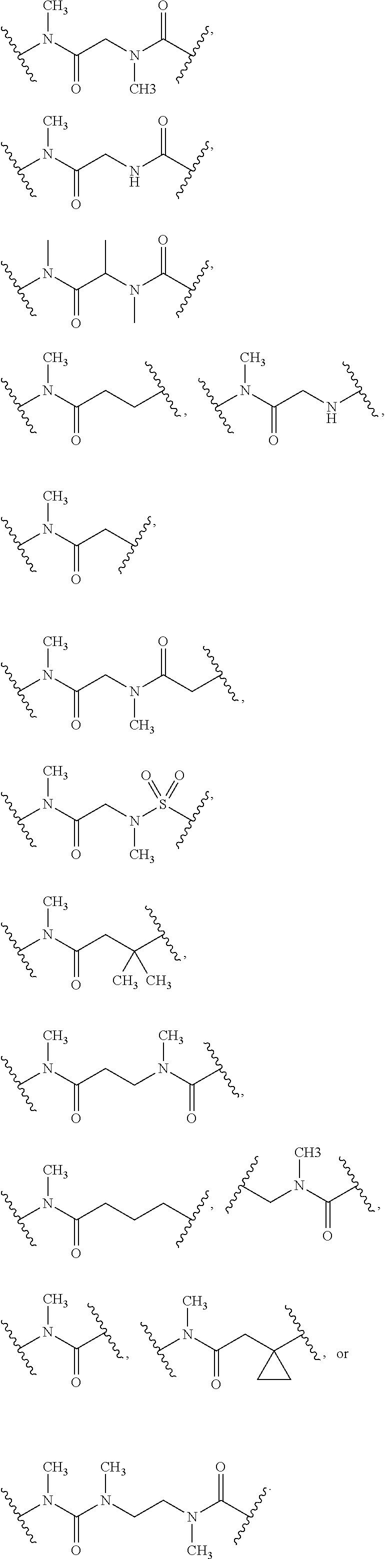

In some embodiments, the linker is or comprises a cyclic group. In some embodiments, the linker has the structure of Formula IIb:

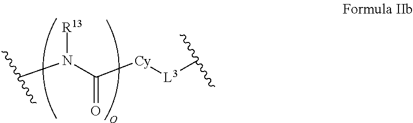

wherein o is 0 or 1; R¹⁵ is hydrogen or optionally substituted C₁-C₆ alkyl; Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and L³ is absent, —SO₂—, optionally substituted C₁-C₄ alkylene or optionally substituted C₁-C₄ heteroalkylene. In some embodiments, the linker has the structure:

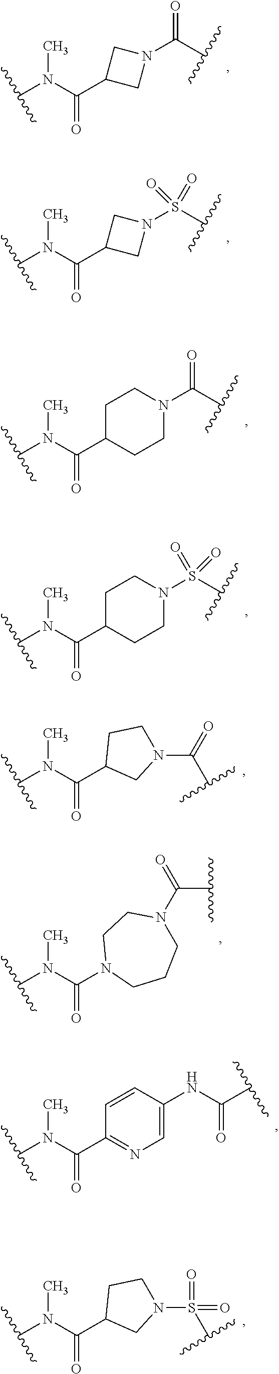

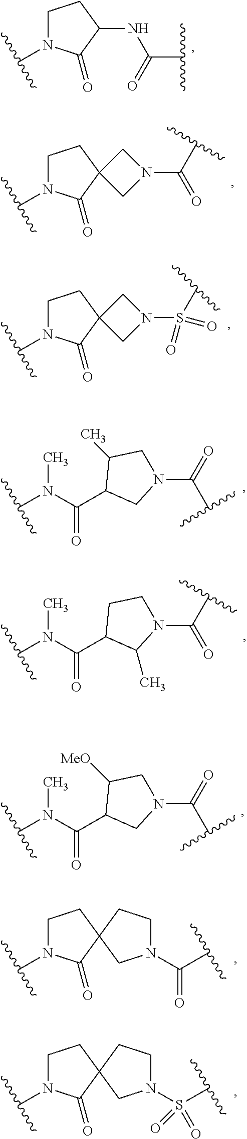

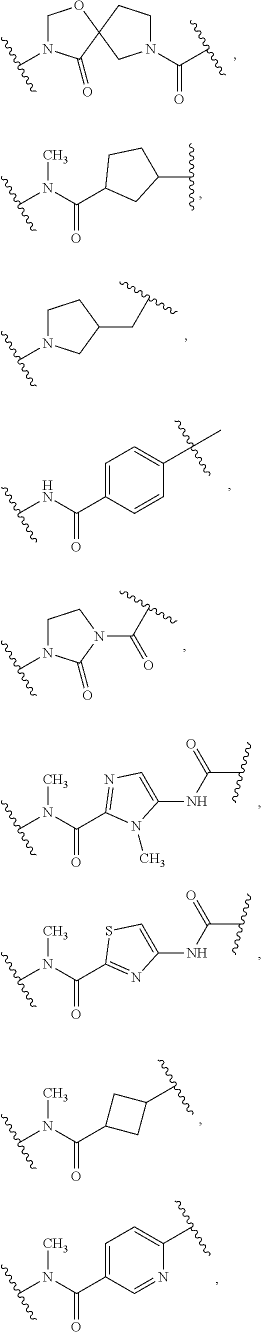

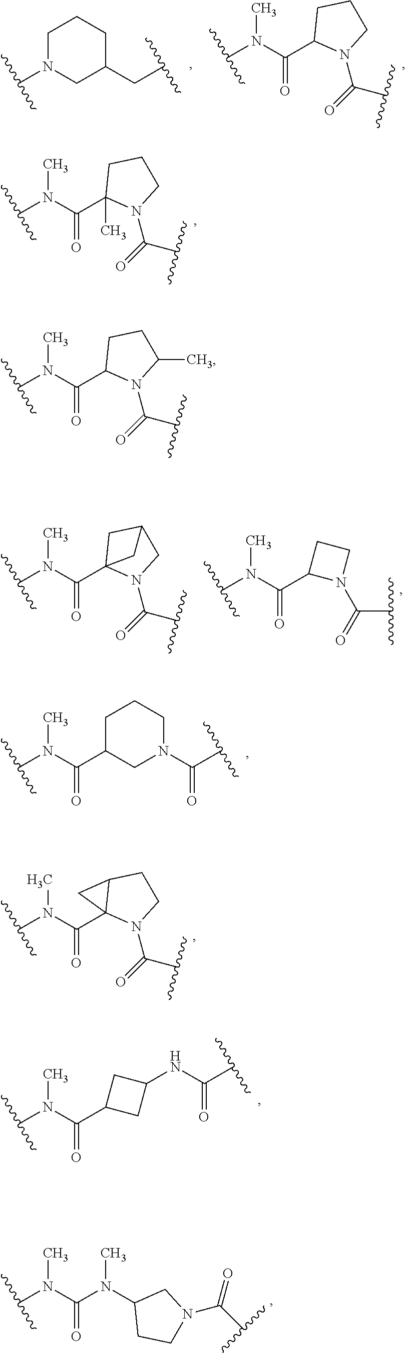

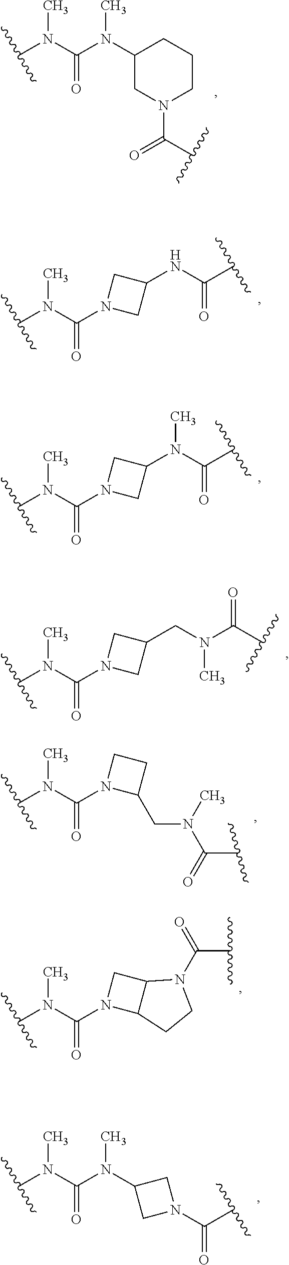

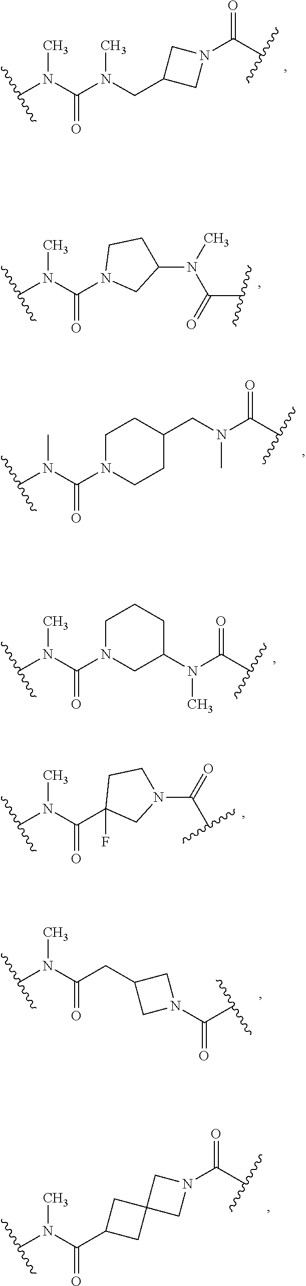

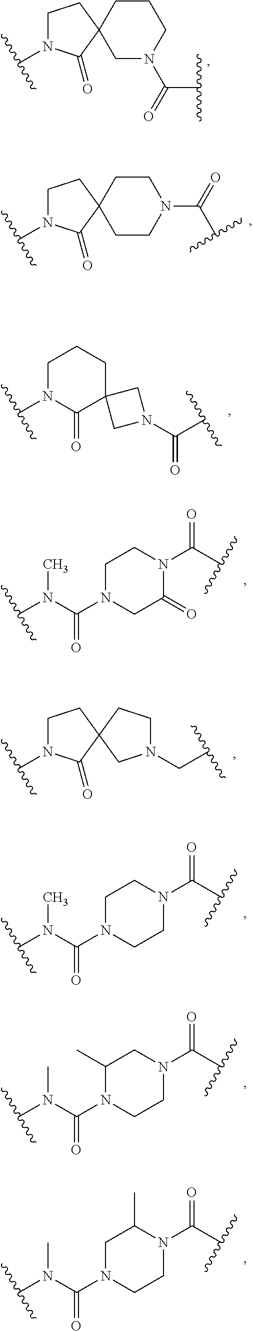

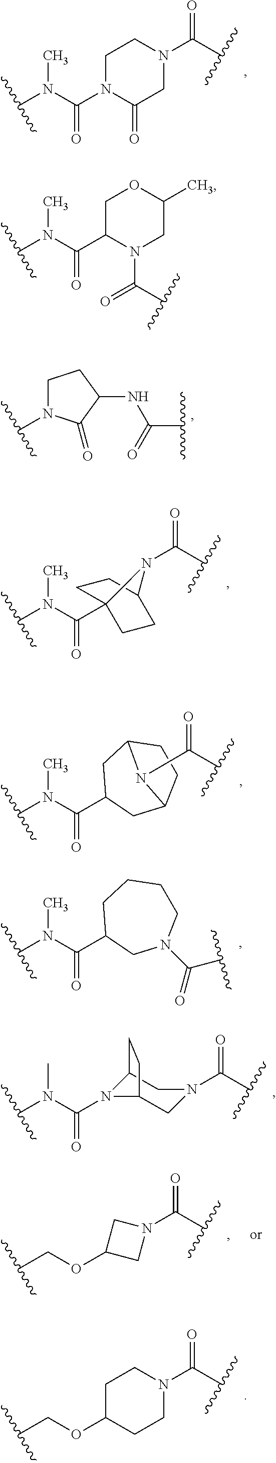

In some embodiments, a linker of Formula II is selected from the group consisting of

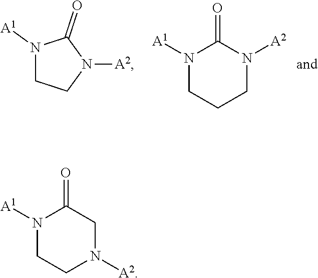

In embodiments, the KRAS G12D inhibitor is

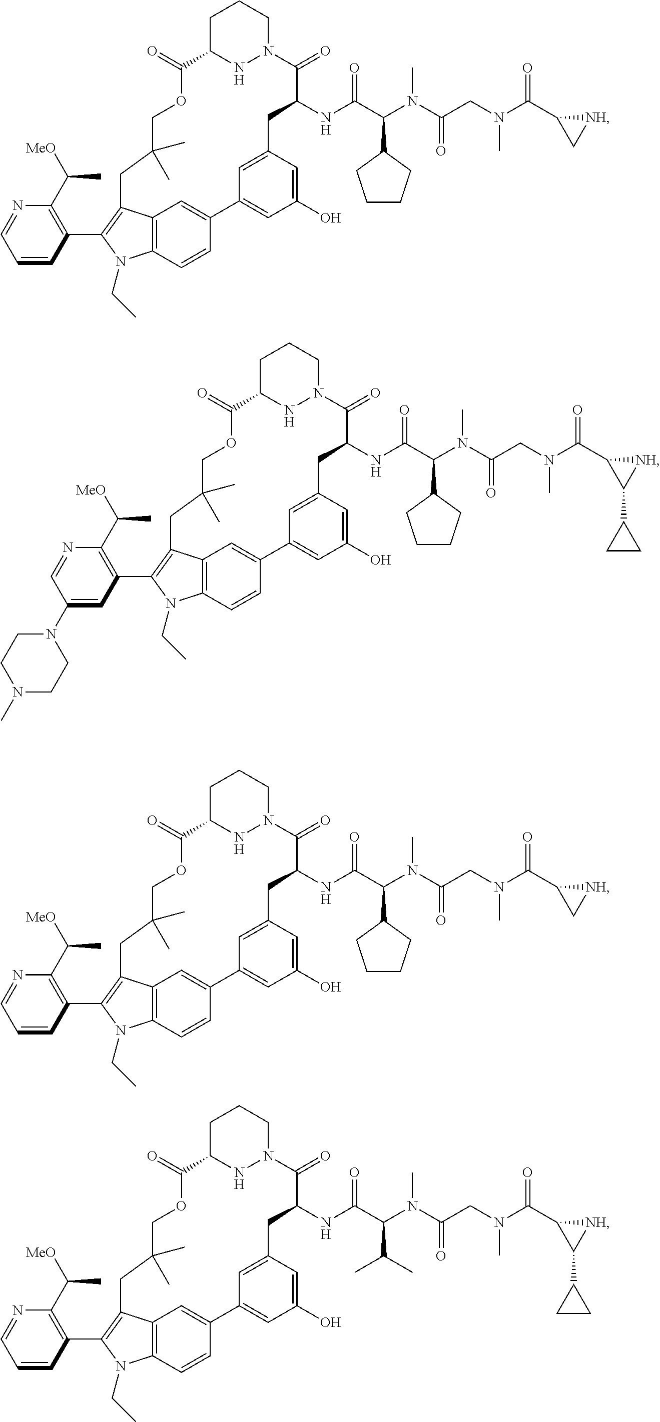

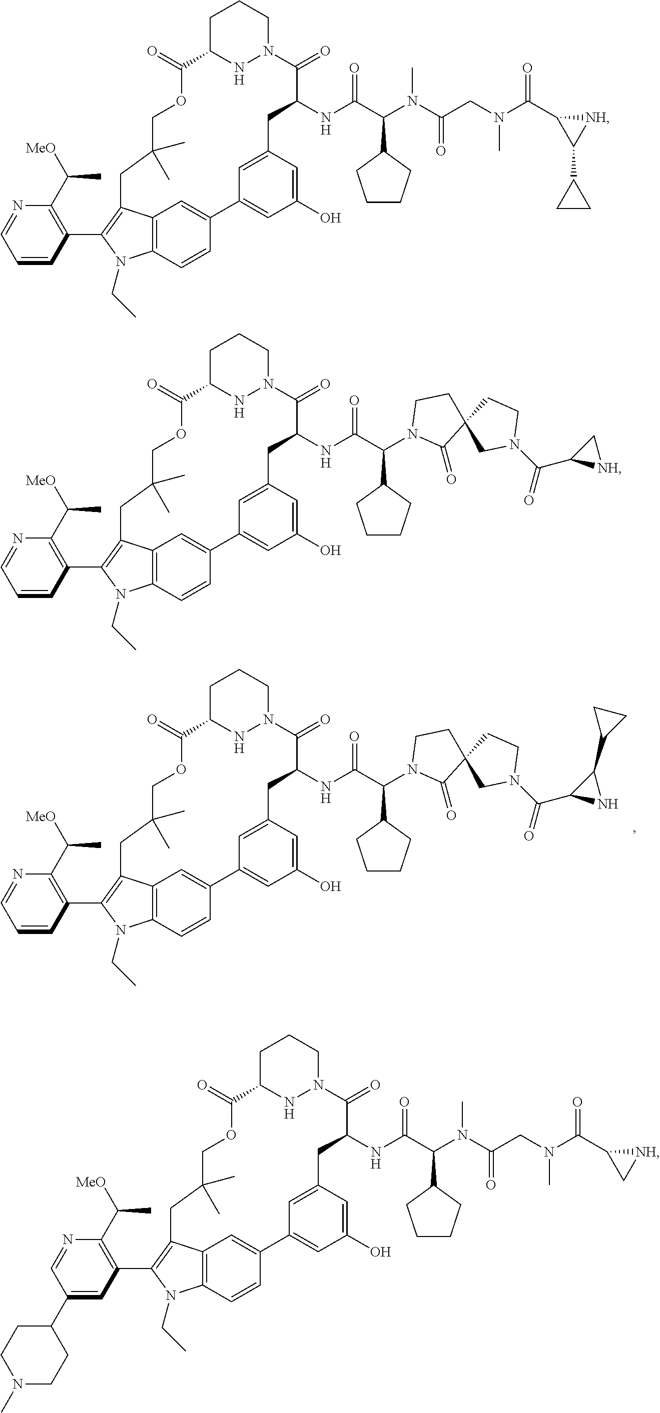

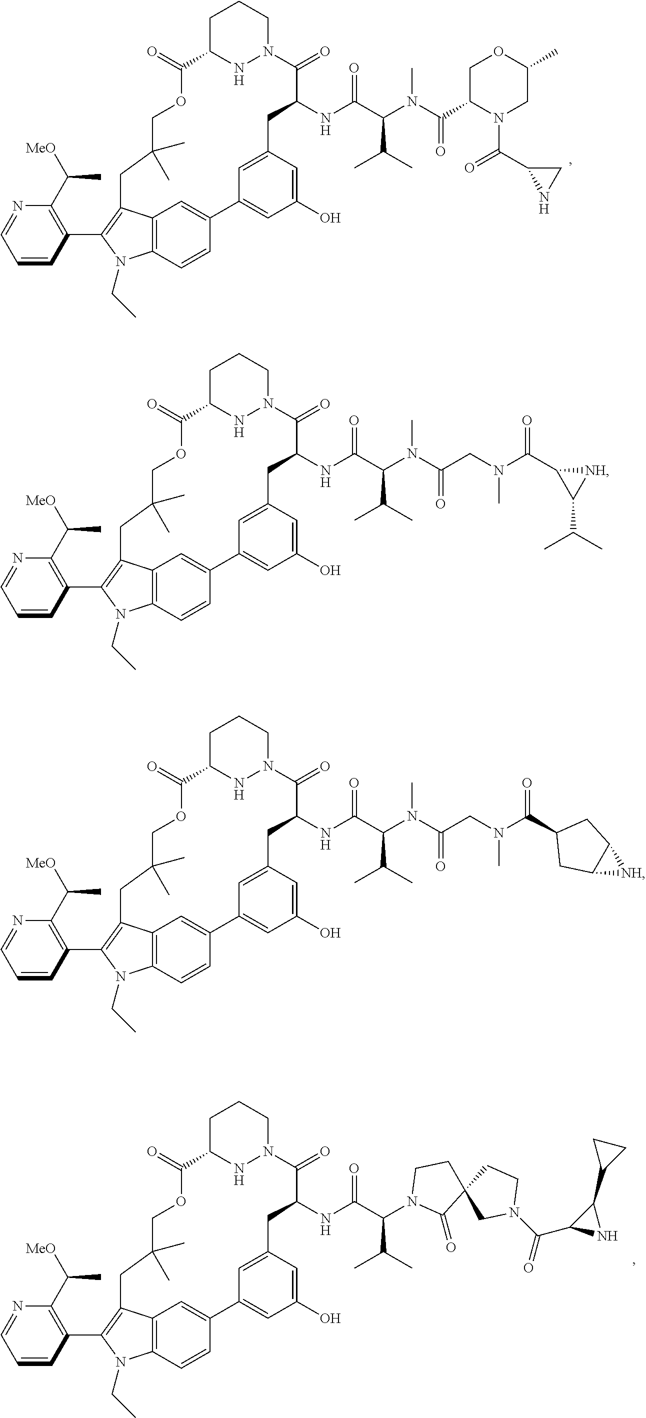

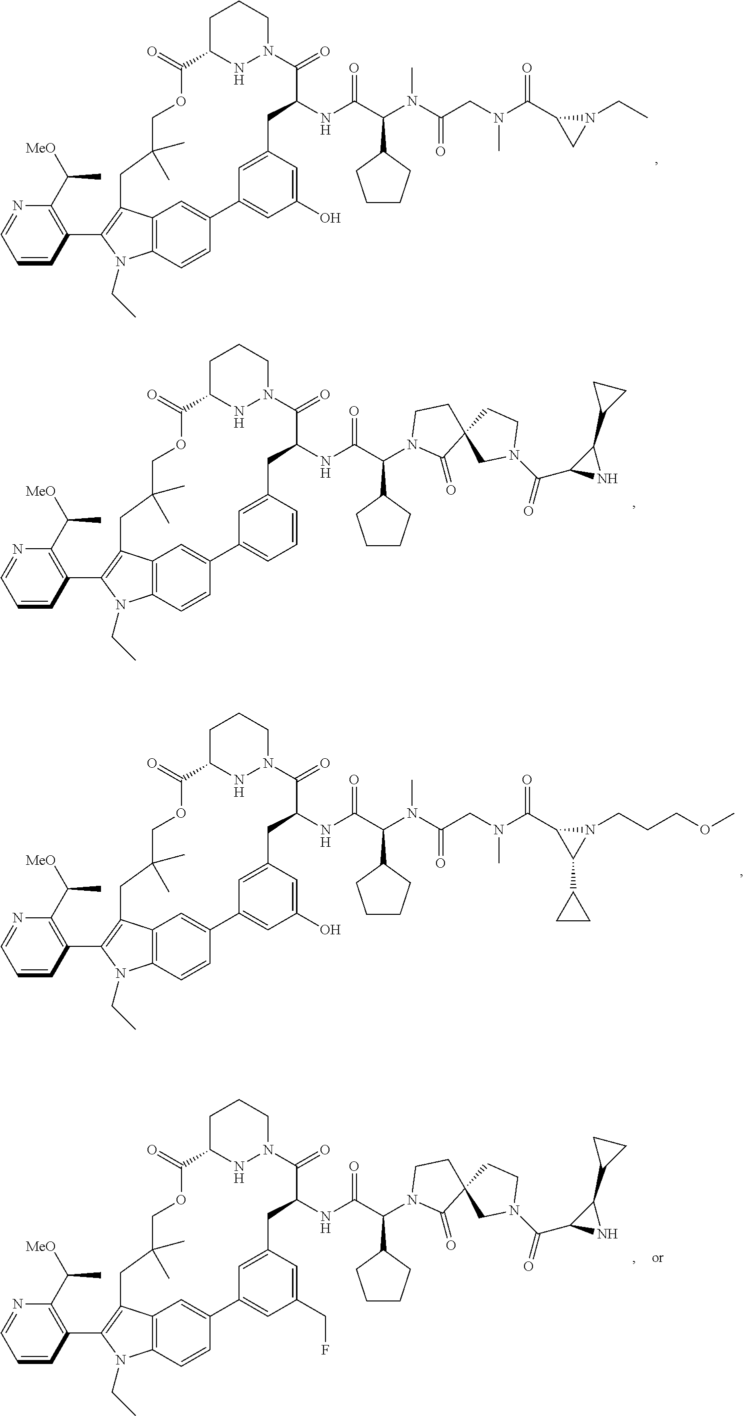

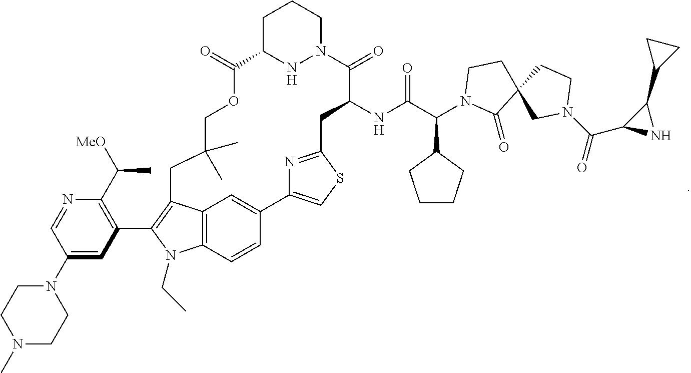

In embodiments, the KRAS G12D inhibitor is a compound recited in Table 6 of WO2021091967, which is incorporated by reference for any purpose, having an IC50 for K-Ras G12D in a AsPC-1 cell viability assay of less than 0.01 μM or less than 0.1 μM and greater than or equal to 0.01 μM or less than 1 uM and greater than or equal to 0.1 μM.

In embodiments, the KRAS G12D inhibitor is a compound exhibiting a pERK EC50 of under 5 μM (AsPC-1 KRAS G12D) in WO2021091967, including those compounds recited by compound number on page 392 of the published application, which is incorporated by reference for any purpose.

In an aspect is provided a KRAS G12D inhibitor having a structure of formula (CI′)

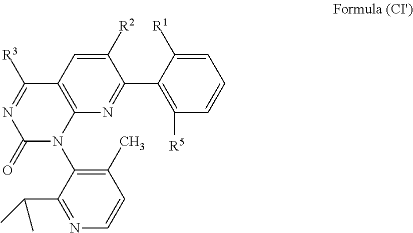

wherein R¹ is H, halo, or —CH₃; R² is H, halo, or —CH₃;

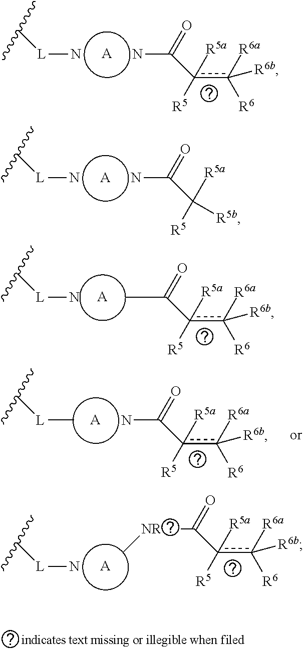

b is optionally a single or a double bond; ring A is a monocyclic 4-7 membered ring or a bicyclic, bridged, fused, or spiro 6-11 membered ring; L is a bond or NR⁴; R⁴ is H, —C₁₋₆alkyl, —C₂₋₆alkynyl, C₁₋₆ alkylene-O—C₁₋₄alkyl, C₁₋₆alkylene-OH, C₁₋₆ haloalkyl, —C₁₋₆ alkyleneamine, —C₀₋₆ alkylene-amide, —C(O)OH, —C(O)OC₁₋₄alkyl, —C₁₋₆ alkylene-O-aryl, —N═N, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl; R⁵ is H, halo, an —C₁₋₆alkyl, —C₂₋₆alkynyl, —C₁₋₆ alkylene-O—C₁₋₄alkyl, —C₁₋₆alkylene-OH, —C₁₋₆ haloalkyl, —C₁₋₆alkyleneamine, —C₀₋₆ alkylene-amide, —C(O)OH, —C(O)OC₁₋₄ alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, —C₀₋₃alkylene-C₂₋₁₄heteroaryl, or cyano; R^{5a} is selected from H, —C₁₋₆alkyl, —C₂₋₆alkynyl, —C₁₋₆ alkylene-O—C₁₋₄alkyl, —C₁₋₆ alkylene-OH, —C₁₋₆haloalkyl, —C₁₋₆alkyleneamine, —C₀₋₆ alkylene-amide, —C(O)OH, —C(O)OC₁₋₄ alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl; R^{5b} is selected from H, —C₁₋₆alkyl, —C₂₋₆alkynyl, —C₁₋₆ alkylene-O—C₁₋₄alkyl, —C₁₋₆ alkylene-OH, —C₁₋₆haloalkyl, —C₁₋₆alkyleneamine, —C₀₋₆ alkylene-amide, —C(O)OH, —C(O)OC₁₋₄ alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl; or R^{5a} and R^{5b} together, may represent an ═O or ═N═N; R⁶ is H, halo, —C₁₋₆alkyl, —C₂₋₆alkynyl, —C₁₋₆ alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl; R^{5a} and R¹, together with the atoms to which they are attached, may form a 3-6 membered ring that optionally includes one or two heteroatoms selected from 0, S or N; or R^{5a} and R^{6a} are absent when b is a double bond; R^{6a} is H, or —C₁₋₆alkyl; R^{6b} is H, —C₁₋₆alkyl, —C₂₋₆alkynyl, —C₁₋₆ alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₁₋₆ alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄ aryl, —C₀₋₃alkylene-C₂₋₁₄heteroaryl, or cyano; or R^{6a} and R⁶ together, may represent an ═O; R⁷ is H or C₁₋₈alkyl; R⁸ is H, OH, NR^{a}R^{b}; wherein R^{a} and R^{b} are each independently H, halo, —C₁₋₆alkyl, —C₂₋₆alkynyl; wherein the ring A or the —C₁₋₆alkyl, —C₂₋₆alkynyl, —C₁₋₆ alkylene-O—C₁₋₄alkyl, —C₁₋₆ alkylene-OH, —C₁₋₆haloalkyl, —C₁₋₆alkyleneamine, —C₀₋₆ alkylene-amide, —C(O)OC₁₋₄alkyl, —C₁₋₆ alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl groups of any of the R⁴, R⁵, R^{5a}, R^{5b}, R⁶, R^{6a}, R^{6b}, R⁷ and R⁸ may be unsubstituted or substituted with 1, 2, 3, or 4 substituents, as allowed, independently selected from halo, —C₁₋₆ alkyl, —O—C₁₋₆alkyl, —OH, or —C₁₋₆alkyl-CN; or a stereoisomer thereof, an atropisomer thereof, a pharmaceutically acceptable salt thereof, a pharmaceutically acceptable salt of the stereoisomer thereof, or a pharmaceutically acceptable salt of the atropisomer thereof. Embodiments described in the present paragraph apply only to the aspects and embodiments of the present paragraph. In embodiments, R³ is selected from

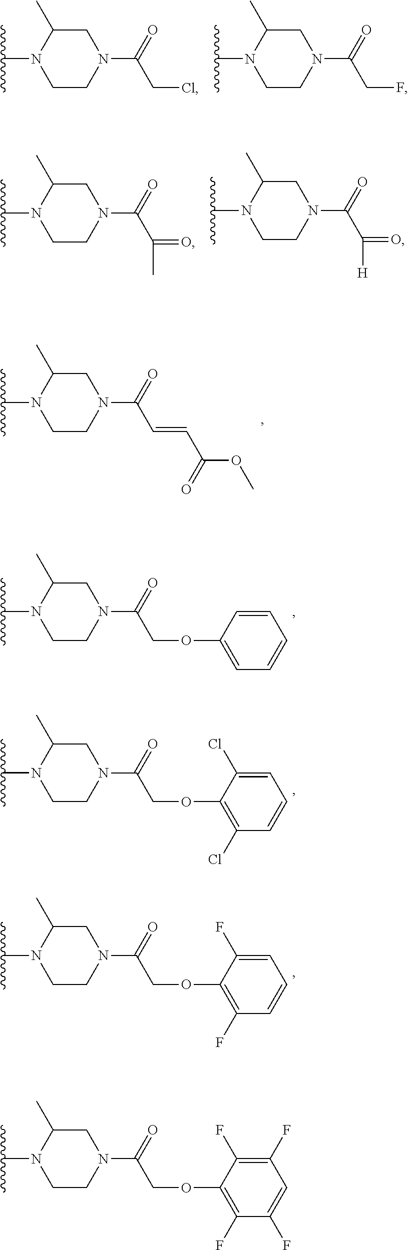

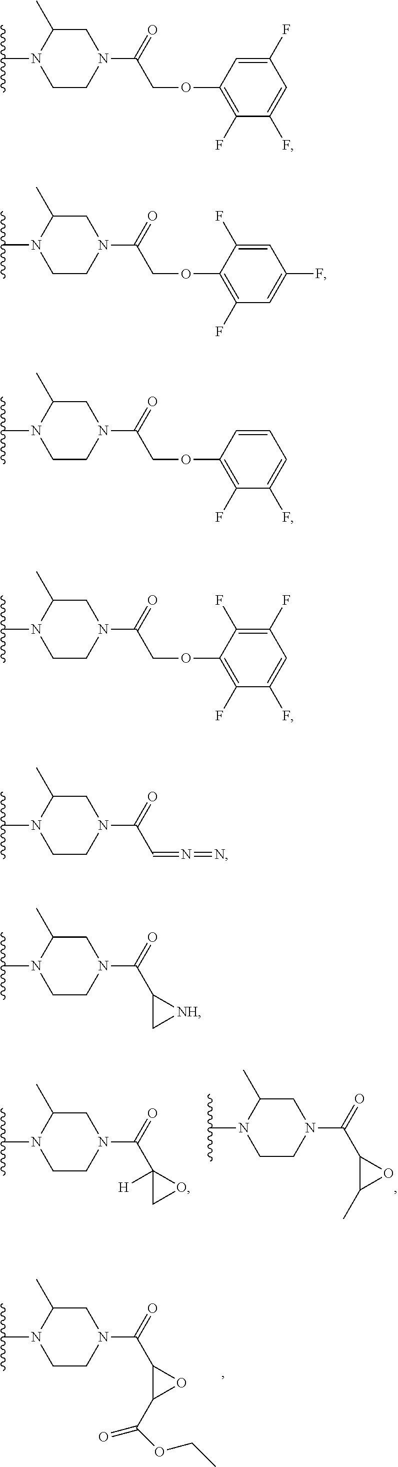

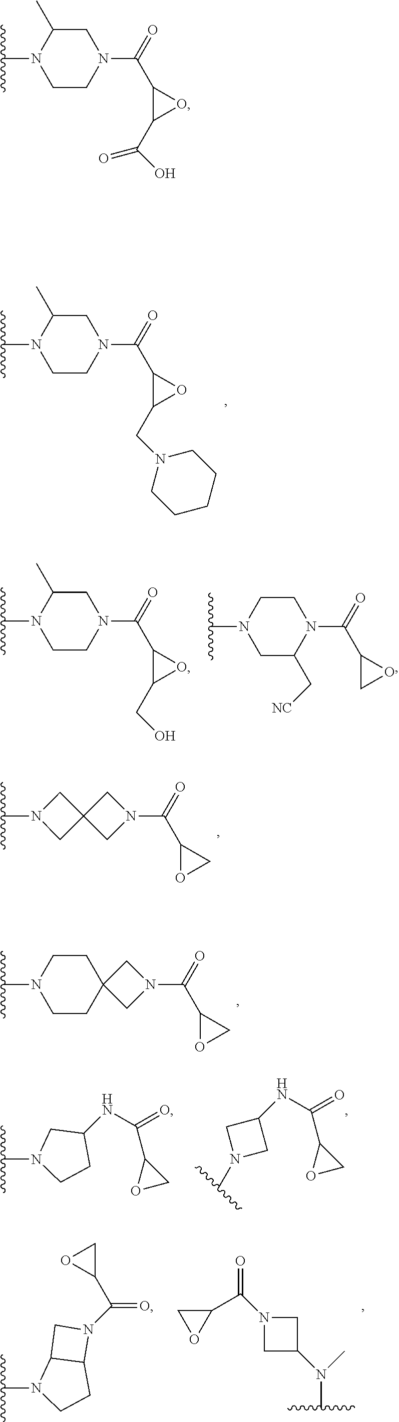

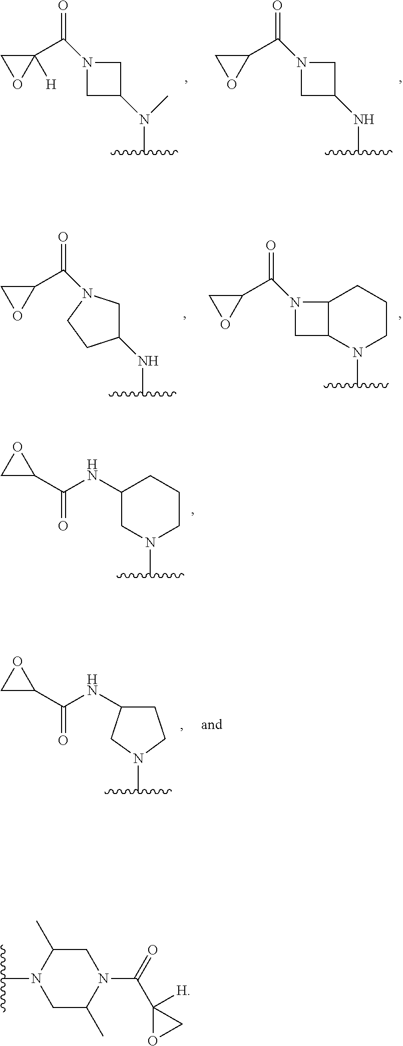

As used in this paragraph, the term “aryl” refers to a C₆₋₁₄ monocyclic or polycyclic aromatic group, preferably a C₆₋₁₀ monocyclic or bicyclic aromatic group, or C₆₋₁₄ polycyclic aromatic group. Examples of aryl groups include, but are not limited to, phenyl, naphthyl, fluorenyl, azulenyl, anthryl, phenanthryl, pyrenyl, biphenyl, and terphenyl. As used in this paragraph Aryl also refers to C₁₀₋₁₄ bicyclic and tricyclic carbon rings, where one ring is aromatic and the others are saturated, partially unsaturated, or aromatic, for example, dihydronaphthyl, indenyl, indanyl, or tetrahydronaphthyl (tetralinyl). Unless otherwise indicated, an aryl group as used in this paragraph can be unsubstituted or substituted with one or more, and in particular one to four, groups independently selected from, for example, halo, C₁₋₆alkyl, C₂₋₆alkenyl, C₂₋₆alkynyl, —CF₃, —OCF₃, —NO₂, —CN, —NC, —OH, alkoxy, amino, —CO₂H, —CO₂C₁-C₆alkyl, —OCOC₁-C₆alkyl, C₃-C₁₀ cycloalkyl, C₃-C₁₀ heterocycloalkyl, C₅-C₁₀aryl, and C₅-C₁₀ heteroaryl. As used in this paragraph, the term “cycloalkyl” refers to a monocyclic or polycyclic non-aromatic carbocyclic ring, where the polycyclic ring can be fused, bridged, or spiro. The carbocyclic ring can have 3 to 10 carbon ring atoms. Contemplated carbocyclic rings include, but are not limited to, cyclopropyl, cyclobutyl, cyclopentyl, cyclohexyl, cycloheptyl, cyclooctyl, and cyclononyl. As used in this paragraph, the term “heterocycloalkyl” means a monocyclic or polycyclic (e.g., bicyclic), saturated or partially unsaturated, ring system containing 3 or more (e.g., 3 to 12, 4 to 10, 4 to 8, or 5 to 7) total atoms, of which one to five (e.g., 1, 2, 3, 4, or 5) of the atoms are independently selected from nitrogen, oxygen, and sulfur. Nonlimiting examples of heterocycloalkyl groups include azetidinyl, pyrrolidinyl, piperidinyl, piperazinyl, dihydropyrrolyl, morpholinyl, thiomorpholinyl, dihydropyridinyl, oxacycloheptyl, dioxacycloheptyl, thiacycloheptyl, and diazacycloheptyl. Unless otherwise indicated, a cycloalkyl or heterocycloalkyl group can be unsubstituted or substituted with one or more, and in particular one to four, groups. Some contemplated substituents include halo, C₁₋₆alkyl, C₂₋₆alkenyl, C₂₋₆alkynyl, —OCF₃, —NO₂, —CN, —NC, —OH, alkoxy, amino, —CO₂H, —CO₂C₁-C₆alkyl, —OCOC₁-C₆alkyl, C₃-C₁₀ cycloalkyl, C₃-C₁₀ heterocycloalkyl, C₅-C₁₀aryl, and C₅-C₁₀ heteroaryl. As used in this paragraph, the term “heteroaryl” refers to a monocyclic or polycyclic ring system (for example, bicyclic) containing one to three aromatic rings and containing one to four (e.g., 1, 2, 3, or 4) heteroatoms selected from nitrogen, oxygen, and sulfur in an aromatic ring. In certain embodiments, the heteroaryl group has from 5 to 20, from 5 to 15, from 5 to 10 ring, or from 5 to 7 atoms. Heteroaryl also refers to C₁₀-14 bicyclic and tricyclic rings, where one ring is aromatic and the others are saturated, partially unsaturated, or aromatic. Examples of heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, triazolyl, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrol opyridyl, quinolinyl, quinoxalinyl, quiazolinyl, thiadiazolopyrimidyl, and thienopyridyl. Unless otherwise indicated, a heteroaryl group can be unsubstituted or substituted with one or more, and in particular one to four or one or two, substituents. Contemplated substituents include halo, C₁₋₆alkyl, C₂₋₆alkenyl, C₂₋₆alkynyl, —OCF₃, —NO₂, —CN, —NC, —OH, alkoxy, amino, —CO₂H, —CO₂C₁-C₆alkyl, —OCOC₁-C₅alkyl, C₃-C₁₀ cycloalkyl, C₃-C₁₀ heterocycloalkyl, C₅-C₁₀aryl, and C₅-C₁₀ heteroaryl. In embodiments, the KRAS G12D inhibitor is selected from

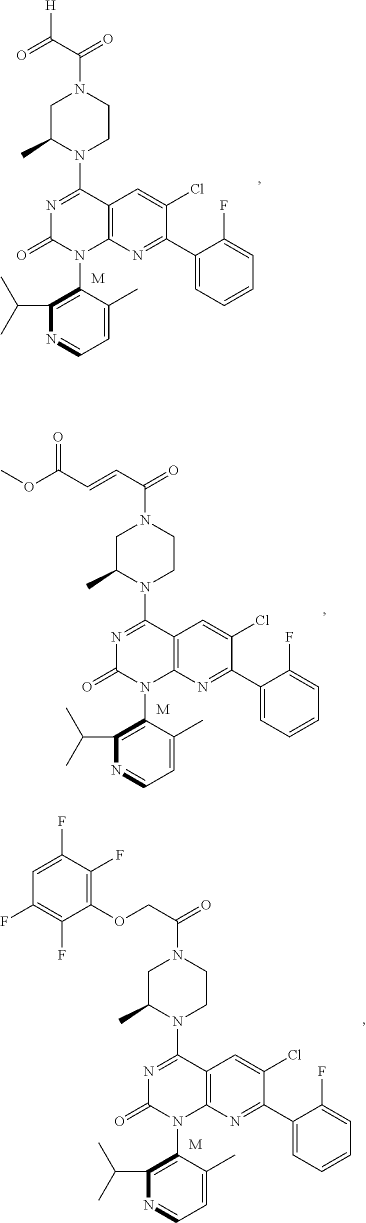

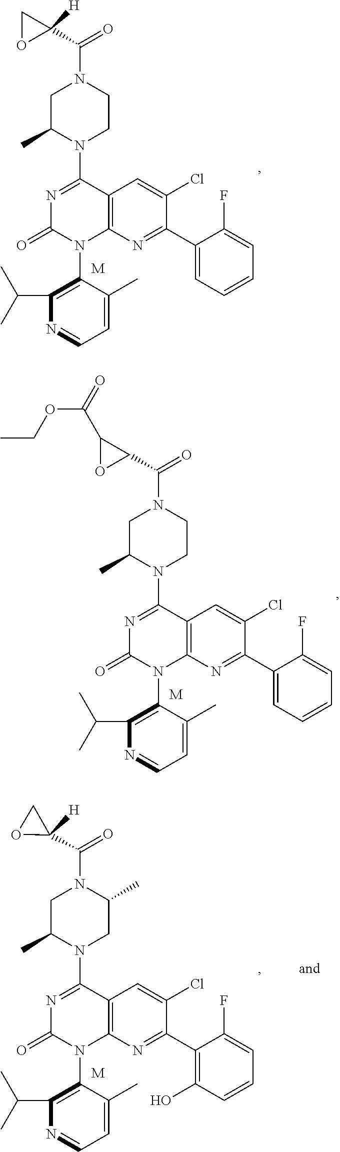

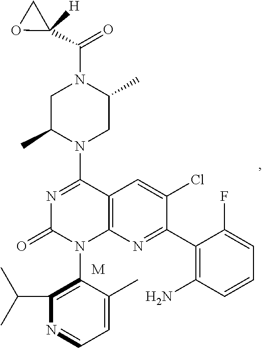

In an aspect, is provided a compound of Formula (CJ′), or a pharmaceutically acceptable salt or solvate thereof:

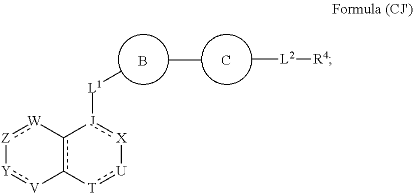

wherein:

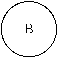

is absent, a 3-12 membered heterocycloalkyl ring, a 6-10 membered aryl ring, a 5-10 membered heteroaryl ring, or a 3-12 membered cycloalkyl ring, wherein the 3-12 membered heterocycloalkyl ring, 6-10 membered aryl ring, 5-10 membered heteroaryl ring, and 3-12 membered cycloalkyl ring are optionally substituted with one or more R¹⁰;

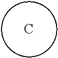

is absent, a 3-12 membered heterocycloalkyl ring, or a 3-12 membered cycloalkyl ring, wherein the 3-12 membered heterocycloalkyl ring and 3-12 membered cycloalkyl ring are optionally substituted with one or more R¹¹;
J is C, C(R^{5a}), or N;
X is C(R⁵), C(O), S(O), S(O)₂, C(R⁵)(R^{a}), O, N, or N(R^{b});
Y is N, N(R^{6a}), O, C(O), S(O), S(O)₂, C(R⁶), or C(R⁶)(R⁷);
Z is N, N(R^{8a}), O, C(O), S(O), S(O)₂, C(R⁸), or C(R⁸)(R⁹);
V is N, N(R^{16a}), O, C(O), S(O), S(O)₂, C(R¹⁶), or C(R¹⁶)(R¹⁷)
W is N, N(R^{18a}), 0, C(O), S(O), S(O)₂, C(R¹⁸), or C(R¹⁸)(R¹⁹);
U is C(R⁵), C(O), S(O)₂, C(R^{5c})(R^{5d}), O, N, or N(R^{5e});
T is C(R⁵), C(O), S(O)₂, C(R^{5f})(R^{5g}), O, N, or N(R^{5h});
L¹ and L² are independently selected from a bond, C₁-C₆alkyl, —O—, —N(R²⁶)—, —C(O)—, —N(R²⁶)C(O)—, —C(O)N(R²⁶)—, —S—, —S(O)₂—, —S(O)—, —S(O)₂N(R²⁶), —S(O)N(R²⁶), —N(R²⁶)S(O)—, —N(R²⁶)S(O)₂—, —OCON(R²⁶)—, and —N(R²⁶)C(O)N(R²⁶)—;
R⁴ is hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20h};
R⁵, R⁶, R⁸, R¹⁶, R¹⁸, R^{5c}, and R⁵ are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20a};
R^{5a}, R⁷, R⁹, R¹⁷, R¹⁹, R^{5d}, and R^{5g} are independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20b};
R^{5b}, R^{6a}, R^{8a}, R^{16a}, R^{18a}, R^{5e}, and R^{5h} are independently selected from hydrogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
each R¹⁰ and each R¹¹ are independently selected from oxo, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d};
each R¹² is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20e};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20f},
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20g};
each R^{20a}, R^{20b}, R^{20c}, R^{20d}, R^{20e}, R^{20f}, R^{20g}, R^{20h}, and R^{20j} are each independently selected from halogen, —CN, C₁₋₆alkyl, C₂₋₆alkenyl, C_{2- 6}alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²⁶ is selected from hydrogen, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20j};
[symbol] indicates a single or double bond such that all valences are satisfied.
   In embodiments

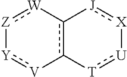

is a quinazoline. In embodiments,

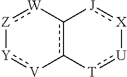

is a pyrido[4,3-d]pyrimidine. In embodiments,

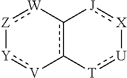

is a quinoline. In embodiments,

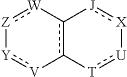

is a pyrido[2,3-d]pyrimidine. In embodiments,

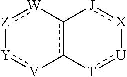

is a pyrido[2,3-d]pyrimidin-2(1H)-one. In embodiments,

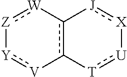

is a 5,8-dihydro-6H-7λ²-pyrido[3,4-d]pyrimidine. In embodiments, the compound of Formula CJ′ is a substituted quinazoline. In embodiments, the compound of Formula CJ′ is a substituted pyrido[4,3-d]pyrimidine. In embodiments, the compound of Formula CJ′ is a substituted quinoline. In embodiments, the compound of Formula CJ′ is a substituted pyrido[2,3-d]pyrimidine. In embodiments, the compound of Formula CJ′ is a substituted pyrido[2,3-d]pyrimidin-2(1H)-one. In embodiments, the compound of Formula CJ′ is a substituted 5,8-dihydro-6H-7λ²-pyrido[3,4-d]pyrimidine.

In some embodiments, the inhibitor of KRas G12C has the structure of Formula T:

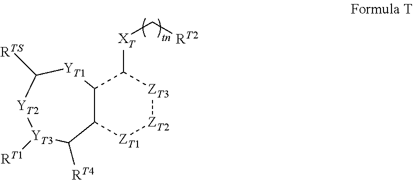

wherein:
R^{T1} is selected from the group consisting of H, C₆₋₁₀ aryl, 5- to 10-membered heteroaryl, and 5- to 10-membered heterocyclyl, wherein each aryl, heteroaryl, and heterocyclyl is optionally substituted with one to four substituents, wherein each substituent is independently selected from the group consisting of C₁₋₆ alkyl, —NH₂, —NH(CH₃), —N(CH₃)₂, halo, C₁₋₆haloalkyl, oxo, C₁₋₆ hydroxyalkyl, C₃₋₆ cycloalkyl, —OC(═O)CH═CH₂, and hydroxy;
R^{T2} is an electrophilic moiety capable of forming a covalent bond with a cysteine residue at position 12 of a K-Ras G12C mutant protein;
Y_{T1} is C(H)(R^{T6}); or Y_{T1} is absent;
Y_{T2} is selected from the group consisting of N(R^{T7}) and C(H)(R^{T8});
Y_{T3} is selected from the group consisting of C(R′³) and N;
Z_{T1} is selected from the group consisting of N, N(R^{T9}), O, S, S(O), and S(O)₂;
Z_{T2} is C(R^{T10}), C(-L^{T}-R^{T10a}), or Z² is absent;
Z_{T3} is selected from the group consisting of N, N(R^{T11}), and C(R^{T12});
R^{T3}, R^{T4}, R^{T5}, R^{T6}, R^{T7}, R^{T8}, R^{T9}, R^{T10}, R^{T11}, and R^{T12} are each independently selected from the group consisting of H, C₂₋₆ alkenyl, C₁₋₆ alkoxy, C₁₋₆ alkyl, C₁₋₆ alkyl substituted with a 4- to 10-membered heterocyclyl substituent, C₁₋₆ alkylsulfanyl, C₁₋₆ alkylsulfonyl, C₁₋₆ alkylthio, C₂₋₆ alkynyl, C₁₋₆ alkylamino, amino, aryl, aryl substituted with a C₁₋₆ alkyl, C₁₋₆ aminoalkyl, carbamoyl, C₁₋₆ carbamoylalkyl, C₁₋₆ carboxyalkyl, cyano, C₁₋₆ cyanoalkyl, C₃₋₇ cycloalkyl, halo, C₁₋₆ haloalkoxy, C₁₋₆ haloalkyl, 5- to 10-membered heteroaryl, 4- to 10-membered heterocyclyl, hydroxy, and oxo;
or R^{T1} and R^{T3}, together with the carbon to which they are bonded, may form an optionally substituted 3- to 6-membered cycloalkyl;
or R^{T3} and R^{T4}, R^{T3} and R^{T8}, R^{T5} and R^{T6}, or R^{T5} and R^{T8}, together with the atoms to which they are each bonded, may form a 3- to 6-membered cycloalkyl or a 3- to 6-membered heterocyclyl;
L^{T} is a bond, O, S, or N(L^{Ta});
R^{T10a} is selected from the group consisting of hydrogen, alkyl, hydroxyalkyl, dihydroxyalkyl, alkylaminylalkyl, dialkylaminylalkyl, -L^{Tb}-NL^{Ta}L^{Tc}, heterocyclyl, heterocyclylalkyl, aryl, heteroaryl, or heteroarylalkyl, wherein each of the L^{Tb}, heterocyclyl, heterocyclylalkyl, aryl, heteroaryl, and heteroarylalkyl may be optionally substituted with one or more L^{Td};
each L^{Ta} is independently hydrogen or C₁₋₃ alkyl;
L^{Tb} is C₁₋₄ alkylene;
each L^{Tc} is independently hydrogen, acyl, C₁₋₃ alkyl, heteroalkyl, or hydroxyalkyl;
each L^{Td} is independently hydrogen, oxo, acyl, hydroxy, hydroxyalkyl, cyano, halogen, C₁₋₆alkyl, aralkyl, haloalkyl, heteroalkyl, cycloalkyl, heterocyclylalkyl, alkoxy, dialkylaminyl, dialkylamidoalkyl, or dialkylaminylalkyl, wherein the C₁₋₆ alkyl may be optionally substituted with cycloalkyl;
XT is selected from the group consisting of C₁₋₆ alkoxy, C₁₋₆ alkyl, amino, C₁₋₆ alkylamino, C₁₋₆ alkylsulfanyl, C₁₋₆ alkylsulfonyl, C₁₋₆ alkylthio, C₃₋₇ cycloalkyl, 4- to 7-membered heterocyclyl, and 4- to 7-membered heterocyclylamino; each of which is optionally substituted with 1 to 4 substituents, wherein each substituent is independently selected from the group consisting of C₁₋₆ alkyl, amino, C₁₋₆ aminoalkyl, carbamoyl, C₁₋₆ carbamoylalkyl, carboxy, C₁₋₆ carboxyalkyl, cyano, C₁₋₆ cyanoalkyl, halo, Cas haloalkyl, hydroxy, C₁₋₆ hydroxyalkyl, and 4- to 7-membered heterocyclyl; wherein two geminal substituents may be taken together to form C₃₋₇ spirocycloalkyl or 4- to 7-membered spiroheterocyclyl;
tn is selected from 0, 1, and 2; and
[symbol] represents a single bond or a double bond.

In some embodiments, the inhibitor of KRas G12C has the structure of Formula U:

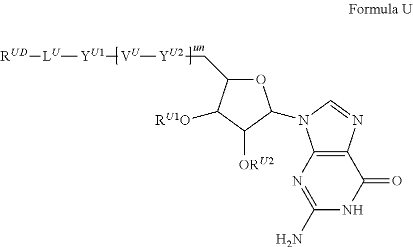

wherein:
each of R^{U1} and R^{U2} is independently hydrogen, optionally substituted alkyl, or an oxygen protecting group, or R^{U1} and R^{U2} are taken together with their intervening atoms to form an optionally substituted heterocyclic ring;
L^{U} is a bond, optionally substituted C₁₋₆ alkylene, —O—, —S—, or —NR^{UN}—;
each instance of Y^{U1} and Y^{U2} is independently —O—, —S—, or —NR^{UN}— or C(R^{UC})₂—;
each instance of V^{U} is independently —C(═O)—, —SO₂—, or

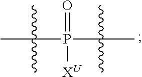

each instance of X^{U} is independently hydrogen, —OR^{UO}, or —NR^{UN1}R^{UN2};
each instance of R^{N}, R^{N1}, and R^{N2} is independently hydrogen, optionally substituted alkyl, or a nitrogen protecting group; or R^{UN1} and R^{UN2} are taken together with their intervening atoms to form an optionally substituted heterocyclic ring;
each instance of R^{UO} is independently hydrogen, optionally substituted alkyl, optionally substituted aryl, or an oxygen protecting group;
each instance of R^{UC} is independently hydrogen, halogen, or optionally substituted alkyl;
un is 1, 2, or 3; and
R^{UD} is selected from

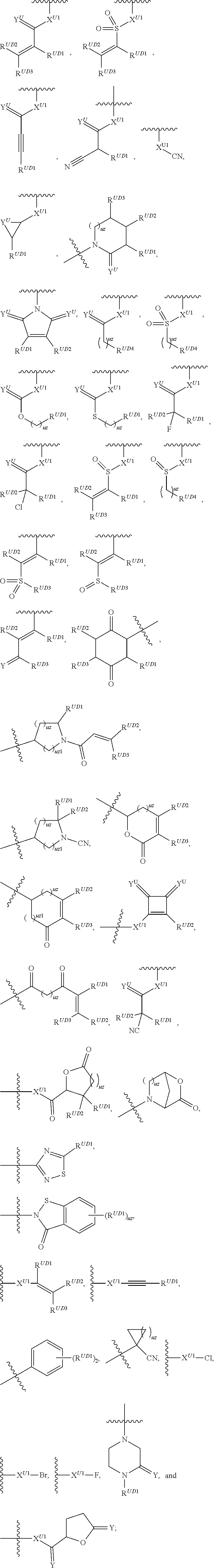

each instance of R^{UD1} is independently hydrogen, halogen, acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —CN, —NO₂, —OR^{UD1a}, —N(R^{UD1a})₂, —SR^{UD1a}, —CH₂OR^{UD1a}, CH₂N(R^{UD1a})₂, —CH₂SR^{UD1a}, —C(═O)R^{UD1a}, —C(═O)OR^{UD1a}, —C(═O)SR^{UD1a}, —C(═O)N(R^{UD1a})₂, —C(═S)R^{UD1a}, —C(═S)OR^{UD1a}, —C(═S)SR^{UD1a}, —C(═S)N(R^{UD1a})₂, —C(═NR^{UD1a})R^{UD1a}, —C(═NR^{UD1a})OR^{UD1a}, C(═N—R^{UD1a})SR^{UD1a}, or —C(═NR^{UD1a})N(R^{UD1a})₂, wherein each occurrence of R^{UD1a} is independently hydrogen, acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{UD1a} groups are joined to form an substituted or unsubstituted heterocyclic ring;
each instance of R^{UD2} is independently hydrogen, halogen, acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted
or unsubstituted aryl, substituted or unsubstituted heteroaryl, —CN, —NO₂, —OR^{UD2a}, —N(R^{UD2a})₂, SR^{UD2a}, —CH₂OR^{UD2a}, —CH₂N(R^{UD2a})₂, —CH₂SR^{UD2a}, —C(═O)R^{UD2a}, —C(═O)OR^{UD2a}, —C(═O)SR^{UD2a}, —C(═O)N(R^{UD2a})₂, —C(═S)R^{UD2a}, —C(═S)OR^{UD2a}, —C(═S)SR^{UD2a}, —C(═S)N(R^{UD2a})₂, —C(═NR^{UD2a})R^{UD2a}, —C(═NR^{UD2a})OR^{UD2a}, —C(═NR^{UD2a})SR^{UD2a}, and —C(═NR^{UD2a})N(R^{UD2a})₂, wherein each occurrence of R^{UD2a} is independently hydrogen, acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl, or two R^{D2a} groups are joined to form an substituted or unsubstituted heterocyclic ring;
each instance of R^{UD3} is independently hydrogen, halogen, acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{UD3a}, —N(R^{UD3a})₂, —SR^{UD3a}, —CH₂OR^{UD3a}, —CH₂N(R^{UD3a})₂, —CH₂SR^{UD3a}, —C(═O)R^{UD3a}, —C(═O)OR^{UD3a}, —C(═O)SR^{UD3a}, —C(═O)N(R^{UD3a})₂, —C(═S)R^{UD3a}, —C(═S)OR^{UD3a}, C(═S)SR^{UD3a}, —C(═S)N(R^{UD3a})₂, —C(═NR^{UD3a})R^{UD3a}, —C(═NR^{UD3a})OR^{UD3a}, C(═NR^{UD3a})SR^{UD3a}, or —C(═NR^{UD3a})N(R^{UD3a})₂ wherein each occurrence of R^{UD3a} is independently hydrogen, acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or two R^{UD3a} groups are joined to form an substituted or unsubstituted heterocyclic ring; optionally R^{UD1} and R^{UD3}, or R^{UD2} and R^{UD3}, or R^{UD1} and R^{UD2} are joined to form an substituted or unsubstituted carbocyclic or substituted or unsubstituted heterocyclic ring;
R^{UD4} is a leaving group selected from the group consisting of —Br, —Cl, —I, and —OS(═O)_{uw}, R^{UD4a}, wherein uw is 1 or 2, and R^{UD4a} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each instance of X^{U1} is independently a bond, —C(═O)—, —SO₂—, NR^{UD5}, optionally substituted alkylene, or optionally substituted heteroarylene, wherein R^{UD5} is hydrogen, C₁₋₆ alkyl, or a nitrogen protecting group;
each instance of Y^{U} is independently O, S, or NR^{UD6}, wherein R^{UD6} is hydrogen, C₁₋₆ alkyl, or a nitrogen protecting group; and
each instance of uz and uz₁ is independently 0, 1, 2, 3, 4, 5, or 6, as valency permits.

In some embodiments, the inhibitor of Ras G12C (e.g., KRas G12C) has the structure of Formula V:

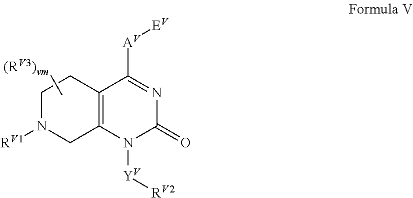

wherein:
each vm is 0, 1 or 2;
A^{V} is a 4-12 membered saturated or partially saturated monocyclic, bicyclic, bridged or spiro ring having a divalent containing 1-2 N atoms, said monocyclic ring, bicyclic ring, bridge ring or spiro ring may be optionally substituted by
one or more R^{V4};
Y^{V} is a bond or C₁₋₆ alkyl;
R^{V1} is aryl or heteroaryl, which may be substituted by 1-3 of the following groups: halogen, hydroxyl, amino, C₁₋₃ alkyl, C₂₋₄ alkenyl, C₃₋₆ cycloalkyl, C₁₋₃ alkoxy, halogen-substituted C₁₋₃ alkyl, or halogen-substituted C₁₋₃ alkoxy;
R^{V2} is aminoalkyl, cycloalkyl, alkyl substituted amido, heterocyclyl, aryl or heteroaryl, wherein said heterocyclyl, aryl or heteroaryl may be optionally substituted by one or more R^{V5};
each R^{V3} is independently selected from C₁₋₃ alkyl and halogenated C₁₋₃ alkyl;
each R^{V4} is independently selected from H, CN, C₁₋₃ alkyl, halogen substituted C₁₋₃ alkyl, and cyano substituted C₁₋₃ alkyl;
each R^{V5} is independently selected from halogen, H, O, CN, OH, alkylhydroxy, dialkylamino, C₁₋₆ alkyl, C₃₋₆ cycloalkyl, halogen-substituted C₁₋₃ alkyl, and halogen-substituted C₁₋₃ alkoxy; and
E^{V} is a cysteine at position 12 capable of interacting with a K-Ras, H-Ras, or N-Ras mutant protein.

In some embodiments, the inhibitor of Ras G12C (e.g., KRas G12C) has the structure of Formula W:

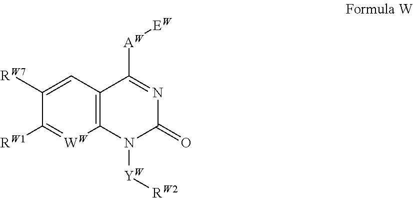

wherein:
A^{W} is a 4-12 membered saturated or partially saturated monocyclic, bicyclic, bridged or spiro ring having a divalent containing 1-2 N atoms, said monocyclic ring, bicyclic ring, bridge ring or spiro ring may be optionally substituted by one or more R^{W4};
Y^{W} is a bond or C₁₋₆ alkyl;
W^{W} is N, —C(R^{W8}), or —C(OR^{W6}); wherein when WW is —C(OR^{W6}), R^{W2} is aminoalkyl, cycloalkyl, alkyl substituted amido, heterocyclyl, aryl or heteroaryl, wherein said heterocyclyl, aryl or heteroaryl are optionally substituted by one or more R^{W5}; wherein when W^{W} is N or —C(R^{W8}), R^{W2} is aminoalkyl, alkyl substituted amido, or heteroaryl optionally substituted by one or more B^{W5};
R^{W1} is aryl or heteroaryl, which may be substituted by 1-3 of the following groups: halogen, hydroxyl, amino, C₁₋₃ alkyl, C₂₋₄ alkenyl, C₃₋₆ cycloalkyl, C₁₋₃ alkoxy, halogen-substituted C₁₋₃ alkyl, or halogen-substituted C₁₋₃ alkoxy;
R^{W2} is aminoalkyl, cycloalkyl, alkyl substituted amido, heterocyclyl, aryl or heteroaryl, wherein said heterocyclyl, aryl or heteroaryl may be optionally substituted by one or more R^{W5};
each R^{W3} is independently selected from C₁₋₃ alkyl and halogenated C₁₋₃ alkyl;
each R^{W4} is independently selected from H, CN, C₁₋₃ alkyl, halogen substituted C₁₋₃ alkyl, and cyano substituted C₁₋₃ alkyl;
each R^{W5} is independently selected from halogen, H, O, CN, OH, alkylhydroxy, dialkylamino, C₁₋₆ alkyl, C₃₋₆ cycloalkyl, halogen-substituted C₁₋₃ alkyl, and halogen-substituted C₁₋₃ alkoxy;
R^{W6} is C₁₋₃ alkyl or C₁₋₆ cycloalkyl;
R^{W7} is H, halogen, C₁₋₃ alkyl, C₃₋₆ cycloalkyl, C₁₋₃ alkoxy, halogen-substituted C₁₋₃ alkyl, halogen-substituted C₁₋₃ alkoxy, or C₂₋₄ alkenyl;
R^{W8} is H, halogen, C₁₋₃ alkyl, C₃₋₆ cycloalkyl, or halogen-substituted C₁₋₆ alkyl; and
E^{W} is a cysteine at position 12 capable of interacting with a K-Ras, H-Ras, or N-Ras mutant protein.

In some embodiments, the compound is a non-covalent binder of RAS, KRAS, HRAS, NRAS, KRAS G12C, KRAS, G12D, HRAS G12C, or NRAS G12C. In some embodiments, the non-covalent binder is as described in US20160264627, WO2015184349, WO2017096045, WO2016172692, WO2017079864, WO2018237084, WO2012153775, WO2015182625, or related patents and applications, each of which is incorporated by reference in its entirety.

In some embodiments, the inhibitor of KRas G12C has the structure of Formula X:

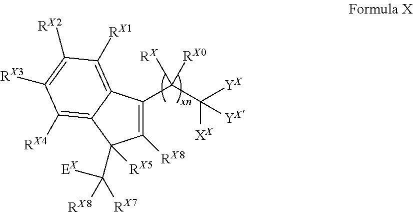

wherein:
R^{X} and R^{X0} are independently selected from hydrogen, hydroxyl, alkyl, cycloalkyl, cycloalkylalkyl, heterocyclyl, heterocyclylalkyl, alkenyl, alkenylalkyl, alkynyl, alkynylalkyl, cyano, cyanoalkyl, halogen, azido, alkoxy, haloalkyl and a substituted or unsubstituted group selected from aminocarbonyl, aminocarbonylalkyl, aminosulfonyl, aminosulfonylalkyl, alkylcarbonyl, alkylcarbonylalkyl, amino, alkylamino, dialkylamino, aminocarbonylalkylamino, and carbocyclylamino, carbocyclylalkylamino, heterocyclylamino and heterocyclylalkylamino wherein the ring structures are saturated or unsaturated; or RX and RX) together is double-bonded oxygen or double-bonded sulfur, or R^{X} and R^{X0} together is a double-bonded nitrogen bonded to one of hydrogen, hydroxyl, alkyl, or haloalkyl, or R^{X} and R^{X0} together is a double-bonded carbon bonded to two substituents independently selected from hydrogen, hydroxyl, alkyl and haloalkyl, or R^{X0} is nitrogen which is part of a substituted or unsubstituted, saturated or unsaturated, 3-, 4-, 5-, 6- or 7-membered heterocyclic ring; or R^{X} and R^{X0} together is a substituted or unsubstituted, saturated or unsaturated 3-, 4-, 5-, 6- or 7-membered heterocyclic ring;
xn is 0, 1 or 2;
R^{X1}, R^{X2}, R^{X3}, and R^{X4} are independently selected from hydrogen, halogen, alkyl, cycloalkyl, haloalkyl, hydroxyl, carboxyl, alkoxy, formyloxy, alkylcarbonyloxy, hydroxyalkyl, aldehydo, amino, aminoalkyl, alkylaminoalkyl, dialkylamino, mercapto, alkylmercapto, cyano, cyanoalkyl, nitro, azido, and substituted or unsubstituted groups selected from alkylsulfinyloxy, alkylsulfonyloxy, carbamate, carbamido, alkoxycarbonyl, alkylaminocarbonyl, aminocarbonyl, alkylcarbonyloxy, alkylcarbonyloxyalkyloxy, aminocarbonyloxyalkyloxy, arylcarbonyloxy, arylalkylcarbonyloxy, aryloxycarbonyloxy, heterocyclylcarbonyloxv, heterocyclylalkylcarbonyloxy, phosphonooxy, phosphonoalkyloxy and sulfonamido;
R^{X5}, R^{X6}, R^{X7}, and R^{X8} are independently selected from hydrogen, alkyl, haloalkyl, and alkoxy; or R^{X5} and R^{X6} together form a carbon-carbon bond;
Y^{X} is hydrogen, alkyl, or haloalkyl, and Y^{X′} is hydrogen, alkyl, haloalkyl, amino, alkylamino, or alkoxy, or Y^{X} and Y^{X′} together is double-bonded oxygen or double-bonded sulfur, or Y^{X} and Y^{X′} together is a double-bonded nitrogen bonded to hydrogen, hydroxyl, alkyl, or haloalkyl;
X^{X} is selected from hydrogen, alkyl, haloalkyl, alkoxy, alkylmercapto, and hydroxyl with the proviso that X^{X} is not hydroxyl when Y^{X} and Y^{X′} together is oxygen in a compound of formula X^{X} wherein R^{X5} and R^{X6} together is a carbon-carbon bond, or X^{X} is NR^{X′}R^{X″}, where R^{X′} is independently selected from the group consisting of hydrogen, hydroxyl, alkyl, aryloxy, cyanoalkyl, haloalkyl, alkoxy, alkenyl, alkynyl, hydroxyalkyl, polyhydroxyalkyl, alkylaminoalkyl, dialkylaminoalkyl, aminoalkyl, alkylamino, aryl, arylalkyl, arylalkenyl, arylcycloalkyl, arylcycloalkenyl, carbocyclyl, and carbocyclylalkyl where the carbocycle of the carbocyclyl and the carbocyclylalkyl is selected from 7-membered carbocyclic rings containing no double bond, or one, two or three double bonds, 6-membered carbocyclic rings containing no double bond, or one, two or three double bonds, 5-membered carbocyclic rings containing no double bond, or one or two double bonds, 4-membered carbocyclic rings containing no double bond or one double bond and 3-membered carbocyclic rings containing no double bond, heterocyclyl, and heterocyclylalkyl, where the heterocycle of the heterocyclyl and heterocyclylalkyl is selected from 7-membered heterocyclic rings, 6-membered heterocyclic rings, and 5-membered heterocyclic rings, and the aryl of the aryl, arylalkyl, arylalkylenyl, arylcycloalkyl, or arylcycloalkenyl structure or the carbocyclic or heterocyclic structure is optionally substituted with one or more of halo, alkyl, trifluoromethyl, hydroxyl, alkoxy, amino, alkylamino, dialkylamino, mercapto, alkylmercapto, carboxamido, aldehydo, cyano, oxo, alkylcarbonyloxy, sulfonamido; R^{X″} is selected from hydrogen, alkyl, hydroxyalkyl, alkylamino, dialkylaminoalkyl, cyanoalkyl, haloalkyl, alkylcarbonylalkylcarbonyloxy, pyridyl, and COR^{X11}, wherein R^{X11} is selected from hydrogen, amino, alkyl, haloalkyl, alkoxy, alkylmercapto, and aryl; or R^{X′} and R^{X″} together form a 5-, 6- or 7-membered, saturated or unsaturated, heterocyclic ring containing at least one nitrogen and optionally oxygen or sulfur, and the heterocyclic ring is optionally substituted with one or more of halo, alkyl, haloalkyl, hydroxyl, alkoxy, amino, alkylamino, dialkylamino, mercapto, alkylmercapto, carboxamido, aldehydo, cyano, oxo, alkylcarbonyloxy, and sulfonamido; and
E^{X} is a substituted or unsubstituted, saturated or unsaturated, 7-membered, 6-membered, 5-membered, 4-membered or 3-membered carbocyclic or heterocyclic ring; or
a pharmaceutically acceptable salt thereof or a prodrug thereof.

In some embodiments, the inhibitor of KRas G12C has the structure of Formula Y:

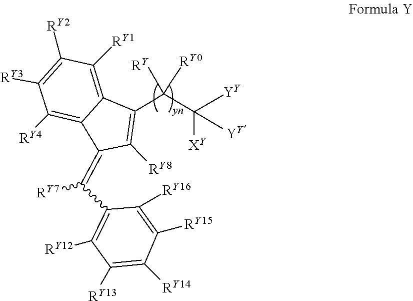

wherein:
R^{Y} and R^{Y0} are independently selected from hydrogen, hydroxyl, alkyl, cycloalkyl, cycloalkylalkyl, heterocyclyl, heterocyclylalkyl, alkenyl, alkenylalkyl, alkynyl, alkynylalkyl, cyano, cyanoalkyl, halogen, azido, alkoxy, haloalkyl and a substituted or unsubstituted group selected from aminocarbonyl, aminocarbonylalkyl, aminosulfonyl, aminosulfonylalkyl, alkylcarbonyl, alkylcarbonylalkyl, amino, alkylamino, dialkylamino, aminocarbonylalkylamino, and carbocyclylamino, carbocyclylalkylamino, heterocyclylamino and heterocyclylalkylamino wherein the ring structures are saturated or unsaturated; or R^{Y} and R^{Y0} together is double-bonded oxygen or double-bonded sulfur, or R^{Y} and R^{Y0} together is a double-bonded nitrogen bonded to one of hydrogen, hydroxyl, alkyl, or haloalkyl, or R^{Y} and R^{Y0} together is a double-bonded carbon bonded to two substituents independently selected from hydrogen, hydroxyl, alkyl and haloalkyl, or R^{Y0} is nitrogen which is part of a substituted or unsubstituted, saturated or unsaturated, 3-, 4-, 5-, 6- or 7-membered heterocyclic ring; or R^{Y} and R^{Y0} together is a substituted or unsubstituted, saturated or unsaturated 3-, 4-, 5-, 6- or 7-membered heterocyclic ring;
yn is 0, 1 or 2;
Y^{Y} is hydrogen, alkyl, or haloalkyl, and Y^{Y′} is hydrogen, alkyl, haloalkyl, amino, alkylamino, or alkoxy, or Y^{Y} and Y^{Y′} together is double-bonded oxygen or double-bonded sulfur, or Y^{Y} and Y^{Y′} together is a double-bonded nitrogen bonded to hydrogen, hydroxyl, alkyl, or haloalkyl;
R^{Y1}, R^{Y2}, R^{Y3}, R^{Y4}, R^{Y12}, R^{Y13}, R^{Y14}, R^{Y15}, and R^{Y16} are independently selected from hydrogen, halogen, alkyl, cycloalkyl, haloalkyl, hydroxyl, carboxyl, alkoxy, formyloxy, hydroxyalkyl, aldehydo, amino, aminoalkyl, alkylaminoalkyl, dialkylamino, mercapto, alkylmercapto, cyano, cyanoalkyl, nitro, azido, and substituted or unsubstituted groups selected from alkylsulfinyloxy, alkylsulfonyloxy, carbamate, carbamido, alkoxycarbonyl, alkylaminocarbonyl, aminocarbonyl, alkylcarbonyloxy, alkylcarbonyloxyalkyloxy, aminocarbonyloxyalkyloxy, arylcarbonyloxy, arylalkylcarbonyloxy, aryloxycarbonyloxy, heterocyclylcarbonyloxy, heterocyclylalkylcarbonyloxy, phosphonooxy, phosphonoalkyloxy and sulfonamido, or any two of R^{Y12}, R^{Y13}, R^{Y14}, R^{Y15}, and R^{Y16} form an alkylenedioxy group;
R^{Y7} and R^{Y8} are independently selected from hydrogen, alkyl, haloalkyl, and alkoxy;
X^{Y} is selected from hydrogen, alkyl, haloalkyl, alkoxy, alkylmercapto, and hydroxyl with the proviso that X^{Y} is not hydroxyl when Y^{Y} and Y^{Y′} together is oxygen, or X^{Y} is NR^{Y′}R^{Y″}, where R^{Y′} is selected from the group consisting of hydrogen, hydroxyl, alkyl, aryloxy, cyanoalkyl, haloalkyl, alkoxy, alkenyl, alkynyl, hydroxyalkyl, polyhydroxyalkyl, alkylaminoalkyl, dialkylaminoalkyl, aminoalkyl, arylalkyl, arylalkenyl, arylcycloalkyl, arylcycloalkenyl, aryl, carbocyclyl, and carbocyclylalkyl where the carbocycle of the carbocyclyl and the carbocyclylalkyl is selected from 7-membered carbocyclic rings containing no double bond, or one, two or three double bonds, 6-membered carbocyclic rings containing no double bond, or one, two, or three double bonds, 5-membered carbocyclic rings containing no double bond, or one or two double bonds, 4-membered carbocyclic rings containing no double bond or one double bond and 3-membered carbocyclic rings containing no double bond, heterocyclyl, and heterocyclylalkyl, where the heterocycle of the heterocyclyl and heterocyclylalkyl is selected from 7-membered heterocyclic rings, 6-membered heterocyclic rings, and 5-membered heterocyclic rings, and the aryl of the aryl, arylalkyl, arylalkylenyl, arylcycloalkyl, or arylcycloalkenyl structure or the carbocyclic or heterocyclic structure is optionally substituted with one or more of halo, alkyl, trifluoromethyl, hydroxyl, alkoxy, amino, alkylamino, dialkylamino, mercapto, alkylmercapto, carboxamido, aldehydo, cyano, oxo, alkylcarbonyloxy, and sulfonamido; and R^{Y″} is selected from hydrogen, alkyl, hydroxyalkyl, aminoalkyl, dialkylaminoalkyl, cyanoalkyl, haloalkyl, alkylcarbonylalkylcarbonyloxy, pyridyl, and COR^{Y11} wherein R^{Y11} is selected from hydrogen, amino, alkyl, haloalkyl, alkoxy, alkylmercapto, and aryl; or R^{Y′} and R^{Y″} together form a 5-, 6- or 7-membered, saturated or unsaturated, heterocyclic ring containing at least one nitrogen, and optionally oxygen and/or sulfur, and the heterocyclic ring is optionally substituted with one or more of halo, alkyl, haloalkyl, hydroxyl, alkoxy, amino, alkylamino, dialkylamino, mercapto, alkylmercapto, carboxamido, aldehydo, cyano, oxo, alkylcarbonyloxy, and sulfonamido; or
a pharmaceutically acceptable salt thereof or a prodrug thereof.

In some embodiments, the inhibitor of KRas G12C has the structure of Formula Z or Z′:

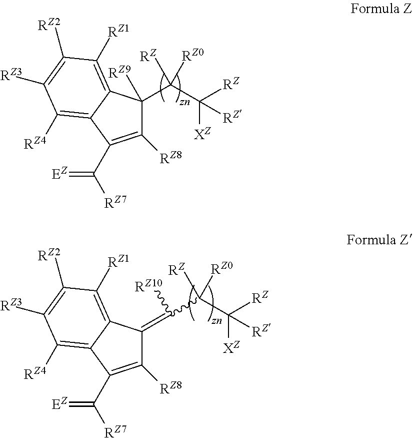

wherein:
R^{Z} and R^{Z0} are independently selected from hydrogen, hydroxyl, alkyl, cycloalkyl, cycloalkylalkyl, heterocyclyl, heterocyclylalkyl, alkenyl, alkenylalkyl, alkynyl, alkynylalkyl, cyano, cyanoalkyl, halogen, azido, alkoxy, haloalkyl and a substituted or unsubstituted group selected from aryl, arylalkyl, aryloxy, aminocarbonyl, aminocarbonylalkyl, aminosulfonyl, aminosulfonylalkyl, alkylcarbonyl, alkylcarbonylalkyl, amino, alkylamino, dialkylamino, aminocarbonylalkylamino, and carbocyclylamino, carbocyclylalkylamino, heterocyclylamino and heterocyclylalkylamino wherein the ring structures are saturated or unsaturated; or R^{Z} and R^{Z0} together is double-bonded oxygen or double-bonded sulfur, or R^{Z} and R^{Z0} together is a double-bonded nitrogen bonded to one of hydrogen, hydroxyl, alkyl, or haloalkyl, or R^{Z} and R^{Z0} together is a double-bonded carbon bonded to two substituents independently selected from hydrogen, hydroxyl, alkyl and haloalkyl, or R^{Z} and R^{Z0} together is a substituted or unsubstituted, saturated or unsaturated 3-, 4-, 5-, 6- or 7-membered heterocyclic ring, or R^{Zo} is nitrogen which is part of a substituted or unsubstituted, saturated or unsaturated, 3-, 4-, 5-, 6- or 7-membered heterocyclic ring;
zn is 0, 1 or 2;
R^{Z1}, R^{Z2}, R^{Z3}, and R^{Z4} are independently selected from hydrogen, halogen, alkyl, cycloalkyl, haloalkyl, hydroxyl, carboxyl, alkoxy, formyloxy, alkylcarbonyloxy, hydroxyalkyl, aldehydo, amino, aminoalkyl, alkylaminoalkyl, dialkylamino, mercapto, alkylmercapto, cyano, cyanoalkyl, nitro, azido, and substituted or unsubstituted groups selected from alkylsulfinyloxy, alkylsulfonyloxy, carbamate, carbamido, alkoxycarbonyl, alkylaminocarbonyl, aminocarbonyl, alkylcarbonyloxy, arylcarbonyloxy, arylalkylcarbonyloxy, aryloxycarbonyloxy, heterocyclylcarbonyloxy, heterocyclylalkylcarbonyloxy, phosphonooxy, phosphonoalkyloxy and sulfamido;
R^{Z7}, R^{Z8}, R^{Z9}, and R^{Z10} are independently selected from hydrogen, alkyl, haloalky, and alkoxy;
Y^{Z} is hydrogen, alkyl, or haloalkyl, and Y^{Z′} is hydrogen, alkyl, haloalkyl, amino, alkylamino, or alkoxy, or Y^{Z} and Y^{Z′} together is double-bonded oxygen or double-bonded sulfur, or Y^{Z} and Y^{Z′} together is a double-bonded nitrogen bonded to hydrogen, hydroxyl, alkyl, or haloalkyl;
X^{Z} is selected from hydrogen, alky, cycloalkyl, haloalkyl, alkoxy, alkylmercapto, and hydroxyl, or X^{Z} is NR^{Z′}R^{Z″}, where R^{Z′}is independently selected from the group consisting of hydrogen, hydroxyl, alkyl, aryloxy, cyanoalkyl, haloalkyl, alkoxy, alkenyl, alkynyl, hydroxyalkyl, polyhydroxyalkyl, alkylaminoalkyl, dialkylaminoalkyl, aminoalkyl, alkylamino, aryl, aryloxy, arylalkyl, arylalkenyl, arylcycloalkyl, arylcycloalkenyl, carbocyclyl, and carbocycloalkyl where the carbocycle of the carbocyclyl and the carbocycloalkyl is selected from 7-membered carbocyclic rings containing no double bond, or one, two or three double bonds, 6-membered carbocyclic rings containing no double bond, or one, two or three double bonds, 5-membered carbocyclic rings containing no double bond, or one or two double bonds, 4-membered carbocyclic rings containing no double bond or one double bond and 3-membered carbocyclic rings containing no double bond, heterocyclyl, and heterocyclylalkyl, where the heterocycle of the heterocyclyl and heterocyclylalkyl is selected from 7-membered heterocyclic rings, 6-membered heterocyclic rings, and 5-membered heterocyclic rings, and the aryl of the aryl, arylalkyl, arylalkylenyl, arylcycloalkyl, or arylcycloalkenyl structure or the carbocyclic or heterocyclic structure may optionally be substituted with one or more of halo, alkyl, haloalkyl, hydroxyl, alkoxy, amino, alkylamino, dialkylamino, mercapto, alkylmercapto, carboxamido, aldehydo, cyano, oxo, alkylcarbonyloxy and sulfonamido; R^{Z″}is selected from hydrogen, alkyl, hydroxyalkyl, alkylamino, dialkylaminoalkyl, cyanoalkyl, haloalkyl and COR^{Z11}, wherein R^{Z11} is selected from hydrogen, amino, alkyl, haloalkyl, alkoxy, alkylmercapto, and aryl; or R^{Z′} and R^{Z″} together form a 5-, 6- or 7-membered, saturated or unsaturated, heterocyclic ring containing at least one nitrogen and optionally oxygen or sulfur, and the heterocyclic ring may optionally be substituted with one or more of halo, alkyl, haloalkyl, hydroxyl, alkoxy, amino, alkylamino, dialkylamino, mercapto, alkylmercapto, carboxamido, aldehydo, cyano, oxo, alkylcarbonyloxy, and sulfonamido; and
E^{Z} is a substituted or unsubstituted, saturated or unsaturated, 7-membered, 6-membered, 5-membered or 4-membered carbocyclic or heterocyclic ring; or
a pharmaceutically acceptable salt thereof.

In some embodiments, the inhibitor of KRas G12C has the structure of Formula AA or Formula AA′:

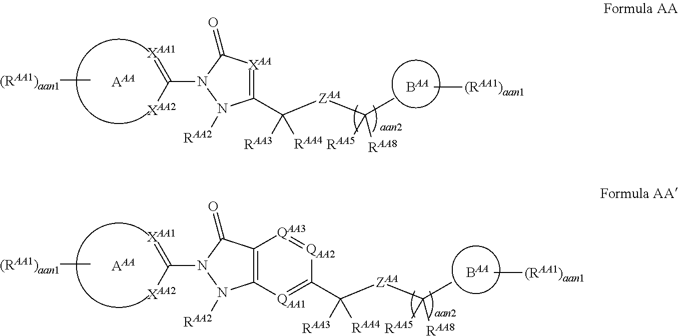

wherein:
A^{AA} is a monocyclic, bicyclic, or tricyclic heterocyclic group;
aan1 is independently 0, 1, 2, 3, 4, 5, 6, 7, or 8;
X^{AA1} and X^{AA2} are each independently CR^{AA1}, O, S, N or NR^{AA2} where valence permits; wherein at least one of X₁ and X₂ is O, N or NR₂;
each occurrence of CR^{AA1} is independently hydrogen, halogen, cyano, nitro, —N₃, CF₃, OCF₃, OR^{AAa}, optionally substituted alkyl, optionally substituted cycloalkyl, optionally substituted alkenyl, optionally substituted alkynyl, optionally substituted aryl, optionally substituted heterocycle, —CR_{b}R_{c}-(optionally substituted aryl), —CR^{AAb}R^{AAc}-(optionally substituted heteroaryl), SR^{AAa}, S(═O)R^{AAa}, S(═O)₂R^{AAa}, —(CR^{AAb}R^{AAc})₁₋₄—NR^{AAb}R^{AAc}, NR^{AAb}R^{AAc}, S(═O)₂NR^{AAb}R^{AAc}, oxo, C(═O)OR^{AAa}, C(═O)R^{AAa}, C(═O)NR^{AAb}R^{AAc}, OC(═O)R^{AAa}, OC(═O)NR^{AAb}R^{AAc}, NR^{AAb}C(═O)OR^{AAA}, or NR^{AAb}C(═O)R^{AAa}; or alternatively two CR^{AA1} groups substituted on the same ring taken together form an additional 3-7 membered carbocycle or heterocycle optionally substituted by one or more CR^{AA1′};
each occurrence of CR^{AA1′}is independently hydrogen, halogen, cyano, nitro, CF₃, OCF₃, ORₐ, optionally substituted alkyl, optionally substituted cycloalkyl, optionally substituted alkenyl, optionally substituted aryl, optionally substituted heterocycle, SR^{AAa}, oxo, S(═O)R^{AAa}, S(═O)₂R^{AAa}, NR^{AAb}R^{AAc}, S(═O)₂NR^{AAb}R^{AAc}, C(═O)OR^{AAa}, C(═O)R^{AAa}, C(═O)NR^{AAb}R^{AAc}, OC(═O)R^{AAa}, OC(═O)NR^{AAb}R^{AAc}, NR^{AAb}C(═O)OR^{AAA}, or NR^{AAb}C(═O)R^{AAa};
each occurrence of CR^{AA2} is independently hydrogen, optionally substituted alkyl, optionally substituted cycloalkyl, optionally substituted alkenyl, optionally substituted cycloalkenyl, optionally substituted alkynyl, optionally substituted heterocycle, or optionally substituted aryl;
X^{AA} is CR^{AA1} or N;
Q_{AA1}, Q_{AA2} and Q^{AA3} are each independently CR^{AA1} or N;
each occurrence of R^{AA3} and R^{AA4} is independently hydrogen, alkyl, cycloalkyl, alkenyl, cycloalkenyl, or alkynyl; or alternatively R^{AA3} and R^{AA4} together with the carbon atom that they are connected to form a 3-7 membered optionally substituted carbocycle or heterocycle;
Z^{AA} is CR^{AA3}R^{AA4}, NR^{AA2}, O, or S;
aan2 is 0 or 1;
each occurrence of R^{AA5}and R^{AA6} is independently hydrogen, alkyl, cycloalkyl, alkenyl, cycloalkenyl, or alkynyl; or alternatively R^{AA5} and R^{AA6} together with the carbon atom that they are connected to form a 3-7 membered optionally substituted carbocycle or heterocycle;
B^{AA} is absent, or cycloalkyl group or saturated heterocyclic group optionally substituted by one or more R^{AA1}, or monocyclic, bicyclic, or tricyclic aryl or heteroaryl group optionally substituted by one or more R^{AA1}; and
R^{AAa}, R^{AAb}, and R^{AAc} are each independently hydrogen, alkyl, cycloalkyl, alkenyl, cycloalkenyl, alkynyl, optionally substituted heterocycle, or optionally substituted aryl; or alternatively R^{AAb}, and R^{AAc} together with the nitrogen atom that they are connected to form an optionally substituted heterocycle comprising the nitrogen atom and 0-3 additional heteroatoms each selected from the group consisting of N, O, and S;
or a pharmaceutically acceptable salt thereof.

### SOS-Binding Molecules

In some embodiments, the inhibitor of SOS is as described in WO2018172250, WO2019201848, WO2018115380, and WO2019122129, or related patents and applications, each of which is incorporated by reference in its entirety.

In some embodiments, the SOS inhibitor has the structure of Formula BB: CH₃

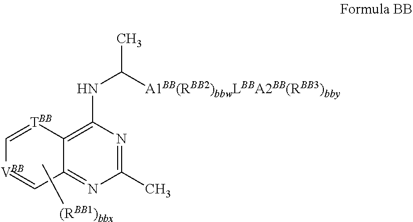

wherein:
R^{BB1} is selected from hydrogen, halogen, hydroxy, cyano, nitro, C₁-C₆-alkylsulfanyl, NR^{BBa}R^{BBb}, C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl, —C(═O)OH, —C(═O)OR^{BBc}, —N═S(═O)(R^{BBd})R^{BBe}, —NH—C(O)—C₁-C₆-alkyl, and —NH—C(O)—NR^{BBf}R^{BBg}, —O—(CH₂)_{bbz}-phenyl, —O—(CH₂)_{bbz}—C₄-C₇-heterocycloalkyl, —O—(CH₂)_{bbz}-heteroaryl,

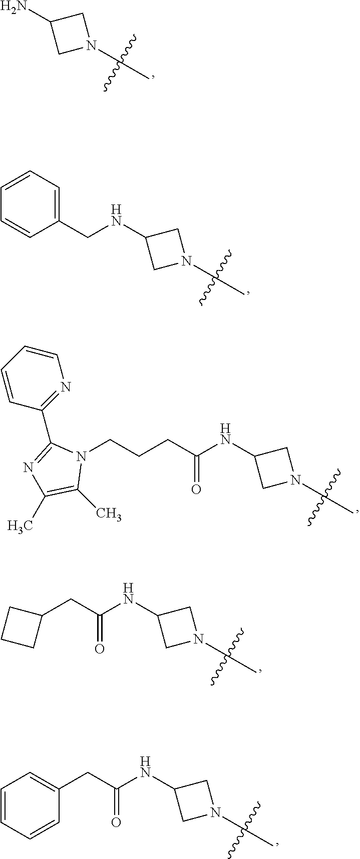

wherein
R^{BBa} and R^{BBb} are selected independently from hydrogen and C₁-C₆-alkyl;
R^{BBc} is C₁-C₆-alkyl, C₃-C₆-alkenyl, C₃-C₆-alkynyl, C₃-C₈-cycloalkyl, or C₄-C₈-cycloalkenyl;
R^{BBd} and R^{BBe} are independently selected from hydrogen, C₁-C₆-alkyl, C₂-C₆-alkenyl, C₂-C₆-alkynyl, C₃-C₈-cycloalkyl, and C₄-C₈-cycloalkenyl;
R^{BBf} and R^{BBg} are selected independently from a hydrogen atom or a C₁-C₆-alkyl, —NH—(CH₂)_{bbk}—NH—C(O)—C₁-C₆-alkyl, and —NH—(CH₂)_{bbi}R^{BBh};
bbk is 1 or 2;
bbi is 0, 1 or 2;
R^{BBh} is a 4- to 7-membered heterocycloalkyl, heteroaryl, or C₁-C₆-alkylsulfonyl;
whereby in all foregoing definitions the C₁-C₆-alkyl-, C₁-C₆-alkoxy-, the 4- to 7-membered heterocycloalkyl and the heteroaryl can be optionally substituted, one, two or three times, identically or differently, with a halogen atom, or a group selected from hydroxy, oxo (═O), a cyano, nitro, C₁-C₆-alkyl, C₂-C₆-alkenyl, C₂-C₆-alkynyl, C₃-C₆-cycloalkyl, 4- to 7-membered heterocycloalkyl, C₁-C₆-alkoxy, C₁-C₆-haloalkyl, C₁-C₆-haloalkoxy-, C₁-C₆-alkylsulfonyl, phenyl, benzyl-, heteroaryl, —(CH₂)-heteroaryl-, C₃-C₈-cycloalkoxy-, phenyloxy-, heteroaryloxy-, —NH—C(O)—C₁-C₆-alkyl or NR^{BBa}R^{BBb};
bbz is 0, 1 or 2, and the phenyl, heterocycloalkyl and heteroaryl can optionally be substituted with a group selected from hydroxy, heterocycloalkyl or heterocycloalkenyl, which both can be substituted with a methyl and/or oxo-group;
bbx is 1, 2 or 3;
A1^{BB} is a C₄ to C₁₂ carbocyclic, heterocyclic, optionally bicyclic, optionally aromatic or optionally heteroaromatic ring system, wherein in a bicyclic aromatic or heteroaromatic ring system one or two double bonds can be hydrogenated,
R^{BB2} is hydrogen, hydroxy, oxo (═O), halogen, cyano, C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, —O—CH₂-4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl, C₁-C₆-alkylsulfonyl, —NR^{BBa}R^{BBb}, —C(O)—NR^{BBa}R^{BBb}, —C(O)—O—R^{BBi};
R^{BBi} a is hydrogen, C₁-C₆-alkyl, OR—O—R^{BBj};
R^{BBj} is a C₁-C₆-alkyl or —CH₂—NR^{BBa}R^{BBb};
bbw is 1 or 2;
A2^{BB}(R^{BB3})_{bby} stands either for a hydrogen atom or
A2^{BB} has the same meanings as the substituent A1^{BB};
R^{BB3} is independently selected from hydrogen, halogen, hydroxy, oxo, cyano, nitro group, C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, and C₁-C₆-haloalkyl, wherein C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, and C₁-C₆-haloalkyl are optionally substituted with one, two or substituents independently selected from halogen, hydroxy, oxo (═O), cyano, C₁-C₆-alkyl, C₁-C₆-alkoxy, C₁-C₆-haloalkyl, phenyl, —C(O)NR^{BBk}R^{BBl};
R^{BBk} and R^{BBl} are selected independently from hydrogen, C₁-C₆-alkyl, heteroaryl, and —NR^{BBm}R^{BBn}, wherein R^{BBm} and R^{BBn} are selected independently from hydrogen, C₁-C₆-alkyl, C₂-C₆-alkenyl, C₂-C₆-alkynyl, C₃-C₈-cycloalkyl, C₁-C₆-alkylsulfonyl, phenyl, heteroaryl, and 4- to 7-membered heterocycloalkyl, which are optionally substituted with a substituent selected from C₁-C₆-haloalkyl, hydroxyl, oxo (═O), phenyl, cyano, C₁-C₆-alkoxy, and heteroaryl, wherein the heteroaryl can optionally be substituted with a methyl group, or —CH₂—C(O)—R^{BBo};
R^{BBo} is a bicyclic heteroaryl, which can be partially hydrogenated, a C₁-C₆-alkoxy or a group —NR^{BBp}R^{BBq};
R^{BBp} and R^{BBq} are selected independently from hydrogen, C₁-C₆-alkyl, and phenyl, wherein the C₁-C₆-alkyl can optionally be substituted with a C₁-C₆-alkoxy or a phenyl, or —NR^{BBp}R^{BBq} is a 4- to 7-membered azacycloalkyl, bound via the nitrogen atom to the rest of the molecule and which optionally contains one more heteroatom selected from nitrogen and oxygen, or —C(═O)R^{BBr};
R^{BBr} is selected from C₁-C₆-alkoxy, and C₁-C₆-alkyl, which is optionally substituted with one, two or three substituents independently selected from hydroxyl, C₁-C₆-alkoxy, a mono- or bicyclic heteroaryl, a 4- to 7-membered heterocycloalkyl or R^{BBr} is a group NR^{BBs}R^{BBt}; wherein Rans and Rant are selected independently from hydrogen, phenyl and C₁-C₆-alkyl, which may optionally be substituted up to threefold with fluorine;
bby is 1, 2 or 3;
L^{BB} is a bond, —O—(F2)_{bbj}, or —CH═CH—(CH₂)_{bbn};
bbj is 0, 1, 2 or 3;
bbn is 0, 1 or 2;
T^{BB} is C(H) or N; and
V^{BB} is C(H) or N;
or a tautomer, an N-oxide, a hydrate, a solvate, or a salt thereof, or a mixture of same.

In some embodiments, the SOS inhibitor has the structure: 6,7-dimethoxy-2-methyl-N-[(1R)-1-(naphthalen-1-yl)ethyl]quinazolin-4-amine; N-[(1R)-1-(3-chlorophenyl)ethyl]-6,7-dimethoxy-2-methylquinazolin-4-amine; methyl 4-{1-[(6,7-dimethoxy-2-methylquinazolin-4-yl)amino]ethyl}-1-benzothiophene-2-carboxylate; N-[1-(1-benzofuran-7-yl)ethyl]-6,7-dimethoxy-2-methylquinazolin-4-amine; N-[1-(7-fluoro-1H-indazol-4-yl)ethyl]-6,7-dimethoxy-2-methylquinazolin-4-amine; N-[1-(6-fluoro-1H-indazol-4-yl)ethyl]-6,7-dimethoxy-2-methylquinazolin-4-amine; 6,7-dimethoxy-2-methyl-N-[1-(5-methyl-1H-indazol-4-yl)ethyl]quinazolin-4-amine; 6,7-dimethoxy-2-methyl-N-[1-(2-methyl-21H-indazol-7-yl)ethyl]quinazolin-4-amine; 6,7-dimethoxy-2-methyl-N-[1-(2-methyl-2H-indazol-4-yl)ethyl]quinazolin-4-amine; 6,7-dimethoxy-2-methyl-N-[1-(1-methyl-1H-indazol-7-yl)ethyl]quinazolin-4-amine; 6,7-dimethoxy-2-methyl-N-[1-(5,6,7,8-tetrahydronaphthalen-1-yl)ethyl]quinazolin-4-amine; N-[(1R)-1-(4-fluorophenyl)ethyl]-6,7-dimethoxy-2-methylquinazolin-4-amine; 6,7-dimethoxy-2-methyl-N-[1-(3-methyl-1H-indazol-4-yl)ethyl]quinazolin-4-amine; N-[1-(1,3-benzothiazol-4-yl)ethyl]-6,7-dimethoxy-2-methylquinazolin-4-amine; N-[1-(1-benzothiophen-7-yl)ethyl]-6,7-dimethoxy-2-methylquinazolin-4-amine; 6,7-dimethoxy-2-methyl-N-[1-(6-methyl-1H-indazol-4-yl)ethyl]quinazolin-4-amine; 6,7-dimethoxy-2-methyl-N-[1-(1-methyl-1H-indazol-4-yl)ethyl]quinazolin-4-amine; N-[1-(5-fluoro-1H-indazol-4-yl)ethyl]-6,7-dimethoxy-2-methylquinazolin-4-amine; N-[1-(2,3-dihydro-1,4-benzodioxin-6-yl)ethyl]-6,7-dimethoxy-2-methylquinazolin-4-amine; N-[1-(1-benzofuran-2-yl)ethyl]-6,7-dimethoxy-2-methylquinazolin-4-anine; N-[1-(2,3-dimethoxyphenyl)ethyl]-6,7-dimethoxy-2-methylquinazolin-4-amine; N-[1-(2,3-dihydro-1-benzofuran-4-yl)ethyl]-6,7-dimethoxy-2-methylquinazolin-4-amine; N-[1-(1,3-benzodioxol-5-yl)ethyl]-6,7-dimethoxy-2-methylquinazolin-4-amine; N-[1-(2,3-dihydro-1-benzofuran-5-yl)ethyl]-6,7-dimethoxy-2-methylquinazolin-4-amine; 6,7-dimethoxy-2-methyl-N-[1-(5,6,7,8-tetrahydronaphthalen-2-yl)ethyl]quinazolin-4-amine; 6,7-dimethoxy-2-methyl-N-[1-(2-methylimidazo[1,2-a]pyridin-3-yl)ethyl]quinazolin-4-amine; N-[1-(1-benzothiophen-3-yl)ethyl]-6,7-dimethoxy-2-methylquinazolin-4-amine; 2-{1-[(6,7-dimethoxy-2-methylquinazolin-4-yl)amino]ethyl}-1-benzofuran-7-ol; 6-bromo-2-methyl-N-[(1R)-1-phenylethyl]quinazolin-4-amine; 6-{[dimethyl(oxido)-lambda6-sulfanylidene]amino}-2-methyl-N-[(1R)-1-phenylethyl]quinazolin-4-amine; 6-bromo-N-[(1R)-1-(4-fluorophenyl)ethyl]-2-methylquinazolin-4-amine 6-{[dimethyl(oxido)-lambda6-sulfanylidene]amino}-N-[(1R)-1-(4-fluorophenyl)ethyl]-2-methylquinazolin-4-amine; 6,7-dimethoxy-N-[1-(7-methoxy-1-benzofuran-2-yl)ethyl]-2-methylquinazolin-4-amine; 6-{1-[(6,7-dimethoxy-2-methylquinazolin-4-yl)amino]ethyl}-2H-1,4-benzoxazin-3(4H)-one; 6,7-dimethoxy-N-[1-(6-methoxy-2-naphthyl)ethyl]-2-methylquinazolin-4-amine; N-[(1R)-1-(5′-amino-2′-methylbiphenyl-3-yl)ethyl]-6,7-dimethoxy-2-methylquinazolin-4-amine; 6,7-dimethoxy-2-methyl-N-{(1R)-1-[3-(pyrimidin-5-yl)phenyl]ethyl}quinazolin-4-anine; N-{(1R)-1-[3′-(cyclopropylmethoxy)biphenyl-3-yl]ethyl}-6,7-dimethoxy-2-methylquinazolin-4-amine; N-{(1R)-1-[3-(isoquinolin-5-yl)phenyl]ethyl}-6,7-dimethoxy-2-methylquinazolin-4-amine; N-[(1R)-1-(2′-chloro-6′-fluoro-3′-methylbiphenyl-3-yl)ethyl]-6,7-dimethoxy-2-methylquinazolin-4-amine; 6,7-dimethoxy-2-methyl-N-{(1R)-1-[3-(5-methylpyridin-3-yl)phenyl]ethyl}quinazolin-4-amine; 6,7-dimethoxy-2-methyl-N-{1-[5-(pyrimidin-5-yl)thiophen-2-yl]ethyl}quinazolin-4-amine; 6,7-dimethoxy-2-methyl-N-[1-{5-[4-(morpholin-4-yl)phenyl]thiophen-2-yl}ethyl]quinazolin-4-amine; 6,7-dimethoxy-2-methyl-N-[1-{5-[3-(morpholin-4-yl)phenyl]thiophen-2-yl}ethyl]quinazolin-4-amine; N-{1-[5-(isoquinolin-5-yl)thiophen-2-yl]ethyl}-6,7-dimethoxy-2-methylquinazolin-4-amine; 6,7-dimethoxy-2-methyl-N-{1-[5-(5-methylpyridin-3-yl)thiophen-2-yl]ethyl}quinazolin-4-amine; 6,7-dimethoxy-2-methyl-N-{1-[5-(2-propoxyphenyl)thiophen-2-yl]ethyl}quinazolin-4-amine; 2-(5-{I-[(6,7-dimethoxy-2-methylquinazolin-4-yl)amino]ethyl}thiophen-2-yl)-N,N-dimethylbenzamide; 6,7-dimethoxy-2-methyl-N-{1-[5-(1-methyl-1H-indo-5-yl)thiophen-2-yl]ethyl}quinazolin-4-anine; 6,7-dimethoxy-N-[1-{5-[2-(methoxymethyl)phenyl]thiophen-2-yl}ethyl]-2-methylquinazolin-4-amine; 3-(5-{1-[(6,7-dimethoxy-2-methylquinazolin-4-yl)amino]ethyl}thiophen-2-yl)-N,N-dimethylbenzamide; (5′-{1-[(6,7-dimethoxy-2-methylquinazolin-4-yl)amino]ethyl}-2,2′-bithiophen-5-yl)methanol; 6,7-dimethoxy-2-methyl-N-{l-[5-(3-methylpyridin-4-yl)thiophen-2-yl]ethyl}quinazolin-4-amine; N-{1-[5-(1H-indol-6-yl)thiophen-2-yl]ethyl}-6,7-dimethoxy-2-methylquinazolin-4-amine; 6,7-dimethoxy-2-methyl-N-{(1R)-1-[3′-(5-methyl-1,3,4-oxadiazol-2-yl)biphenyl-3-yl]ethyl}quinazolin-4-anine; 6,7-dimethoxy-2-methyl-N-[(1R)-1-{3-[5-(methylsulfonyl)pyridin-3-yl]phenyl}ethyl]quinazolin-4-amine; 5-(3-{(1R)-1-[(6,7-dimethoxy-2-methylquinazolin-4-yl)amino]ethyl}phenyl)-1,3-dihydro-2H1-indol-2-one; N-{(1R)-1-[3-(2,2-dimethylcyclopropyl)phenyl]ethyl}-6,7-dimethoxy-2-methylquinazolin-4-amine; 6,7-dimethoxy-2-methyl-N-{(1R)-1-[4′-(5-methyl-1,3,4-oxadiazol-2-yl)biphenyl-3-yl]ethyl}quinazolin-4-amine; 6,7-dimethoxy-2-methyl-N-{(1R)-1-[3-(1H-pyrrolo[2,3-b]pyridin-5-yl)phenyl]ethyl}quinazolin-4-amine; 3′-{(1R)-1-[(6,7-dimethoxy-2-methylquinazolin-4-yl)amino]ethyl}biphenyl-3-sulfonamide; N-{(1R)-1-[3-(2-aminopyrimidin-5-yl)phenyl]ethyl}-6,7-dimethoxy-2-methylquinazolin-4-amine; N-[(1R)-1-{3-[(E)-2-cyclopropylethynyl]phenyl}ethyl]-6,7-dimethoxy-2-methylquinazolin-4-amine; N-{(1R)-1-[2′-(ethoxymethyl)biphenyl-3-yl]ethyl}-6,7-dimethoxy-2-methylquinazolin-4-amine; N-[(1R)-1-(3′-fluoro-5′-methoxybiphenyl-3-yl)ethyl]-6,7-dimethoxy-2-methylquinazolin-4-amine; 6,7-dimethoxy-N-{(1R)-1-[3-(5-methoxy-1-benzofuran-2-yl)phenyl]ethyl}-2-methylquinazolin-4-amine; N-[(1R)-1-(2′-butoxy-6′-fluorobiphenyl-3-yl)ethyl]-6,7-dimethoxy-2-methylquinazolin-4-amine; 5-(3-{(1R)-1-[(6,7-dimethoxy-2-methylquinazolin-4-yl)amino]ethyl}phenyl)pyridin-2-ol; 2-(3′-{(1R)-1-[(6,7-dimethoxy-2-methylquinazolin-4-yl)amino]ethyl}biphenyl-4-yl)-2-methylpropanenitrile; 6,7-dimethoxy-2-methyl-N-[1-(5-phenylthiophen-2-yl)ethyl]quinazolin-4-amine; N-[(3′-{(1R)-1-[(6,7-dimethoxy-2-methylquinazolin-4-yl)amino]ethyl}biphenyl-3-yl)methyl]methanesulfonamide; N-[(3′-{(1R)-1-[(6,7-dimethoxy-2-methylquinazolin-4-yl)amino]ethyl}biphenyl-4-yl)methyl]methanesulfonamide; 3′-{(1R)-1-[(6,7-dimethoxy-2-methylquinazolin-4-yl)amino]ethyl}-N-propylbiphenyl-4-carboxamide; 3′-{(1R)-1-[(6,7-dimethoxy-2-methylquinazolin-4-yl)amino]ethyl}-N-[2-(dimethylamino)ethyl]biphenyl-4-carboxamide; 6,7-dimethoxy-2-methyl-N-{(1R)-1-[3-(1H-pyrazol-3-yl)phenyl]ethyl}quinazolin-4-amine; N-[(1R)-1-{3-[(2E)-but-2-en-2-yl]phenyl}ethyl]-6,7-dimethoxy-2-methylquinazolin-4-amine; N-[(1R)-1-(5′-chloro-2′-propoxybiphenyl-3-yl)ethyl]-6,7-dimethoxy-2-methylquinazolin-4-amine; 6,7-dimethoxy-2-methyl-N-[(1R)-1-{3-[(1E)-3-phenylprop-1-en-1-yl]phenyl}ethyl]quinazolin-4-amine; 6,7-dimethoxy-2-methyl-N-{(1R)-1-[4′-(morpholin-4-yl)biphenyl-3-yl]ethyl}quinazolin-4-amine; 6,7-dimethoxy-2-methyl-N-{(1R)-1-[3′-(morpholin-4-yl)biphenyl-3-yl]ethyl}quinazolin-4-amine; N-{(1R)-1-[2′-(benzyloxy)biphenyl-3-yl]ethyl}-6,7-dimethoxy-2-methylquinazolin-4-amine; 6,7-dimethoxy-2-methyl-N-{(1R)-1-[4′-(methylsulfonyl)biphenyl-3-yl]ethyl}quinazolin-4-amine; 6,7-dimethoxy-2-methyl-N-{(1R)-1-[2′-(trifluoromethoxy)biphenyl-3-yl]ethyl}quinazolin-4-amine; 6,7-dimethoxy-2-methyl-N-{(1R)-1-[3′-(trifluoromethoxy)biphenyl-3-yl]ethyl}quinazolin-4-amine; N-{(1R)-1-[3-(1T-indol-5-yl)phenyl]ethyl}-6,7-dimethoxy-2-methylquinazolin-4-amine; N-{(1R)-1-[3-(furan-3-yl)phenyl]ethyl}-6,7-dimethoxy-2-methylquinazolin-4-amine; N-{(1R)-1-[3-(1-benzothiophen-3-yl)phenyl]ethyl}-6,7-dimethoxy-2-methylquinazolin-4-amine; 6,7-dimethoxy-2-methyl-N-{(1R)-1-[3-(1-methyl-1H-indol-2-yl)phenyl]ethyl}quinazolin-4-amine; 6,7-dimethoxy-2-methyl-N-[(1R)-1-{3-[(1E)-pent-1-en-1-yl]phenyl}ethyl]quinazolin-4-amine; N-[(1R)-1-{3-[(E)-2-cyclohexylethenyl]phenyl}ethyl]-6,7-dimethoxy-2-methylquinazolin-4-anine; 6,7-dimethoxy-2-methyl-N-[(1R)-1-(2′-phenoxybiphenyl-3-yl)ethyl]quinazolin-4-amine; tert-butyl (3′-{(1R)-1-[(6,7-dimethoxy-2-methylquinazolin-4-yl)amino]ethyl}biphenyl-4-yl)carbamate; (2E)-3-(3′-{(1R)-1-[(6,7-dimethoxy-2-methylquinazolin-4-yl)amino]ethyl}biphenyl-3-yl)prop-2-enenitrile; N-[(1R)-1-(2′,4′-dimethylbiphenyl-3-yl)ethyl]-6,7-dimethoxy-2-methylquinazolin-4-amine; 1-[5-(3-{(1R)-1-[(6,7-dimethoxy-2-methylquinazolin-4-yl)amino]ethyl}phenyl)thiophen-2-yl]ethenone; N-{(1R)-1-[3-(1,3-benzodioxol-5-yl)phenyl]ethyl}-6,7-dimethoxy-2-methylquinazolin-4-amine; N-{(1R)-[4′-(benzyloxy)biphenyl-3-yl]ethyl}-6,7-dimethoxy-2-methylquinazolin-4-amine; N-{1-[5-(2,3-dihydro-1,4-benzodioxin-6-yl)thiophen-2-yl]ethyl}-6,7-dimethoxy-2-methylquinazolin-4-amine; 6,7-dimethoxy-N-[(1R)-1-(3′-methoxybiphenyl-3-yl)ethyl]-2-methylquinazolin-4-amine; 6,7-dimethoxy-2-methyl-N-{(1R)-1-[3′-(trifluoromethyl)biphenyl-3-yl]ethyl}quinazolin-4-amine; 3′-{(1R)-1-[(6,7-dimethoxy-2-methylquinazolin-4-yl)amino]ethyl}-N,N-dimethylbiphenyl-2-sulfonamide; 6,7-dimethoxy-2-methyl-N-[(1R)-1-(2′-propoxybiphenyl-3-yl)ethyl]quinazolin-4-amine; 3′-{(1R)-1-[(6,7-dimethoxy-2-methylquinazolin-4-yl)amino]ethyl}-N,N-dimethylbiphenyl-2-carboxamide; 6,7-dimethoxy-N-{(1R)-1-[2′-(methoxymethyl)biphenyl-3-yl]ethyl}-2-methylquinazolin-4-amine; 6,7-dimethoxy-2-methyl-N-{(1R)-1-[3-(1-methyl-1H-indol-5-yl)phenyl]ethyl}quinazolin-4-amine; 3′-{(1R)-1-[(6,7-dimethoxy-2-methylquinazolin-4-yl)amino]ethyl}-N,N-dimethylbiphenyl-3-carboxamide; [5-(3-{(1R)-1-[(6,7-dimethoxy-2-methylquinazolin-4-yl)amino]ethyl}phenyl)thiophen-2-yl]methanol; 6,7-dimethoxy-2-methyl-N-{(1R)-1-[3-(3-methylpyridin-4-yl)phenyl]ethyl}quinazolin-4-amine; N-{(1R)-1-[3-(1H-indol-6-yl)phenyl]ethyl}-6,7-dimethoxy-2-methylquinazolin-4-amine; N-{(1R)-1-[3-(1H-indol-4-yl)phenyl]ethyl}-6,7-dimethoxy-2-methylquinazolin-4-amine; 6,7-dimethoxy-N-{(1R)-1-[3-(2-methoxypyrimidin-5-yl)phenyl]ethyl}-2-methylquinazolin-4-amine; 6,7-dimethoxy-2-methyl-N-{(1R)-1-[3′-(methylsulfonyl)biphenyl-3-yl]ethyl}quinazolin-4-amine; N-{(1R)-1-[3-(2,3-dihydro-1-benzofuran-5-yl)phenyl]ethyl}-6,7-dimethoxy-2-methylquinazolin-4-amine; 6,7-dimethoxy-2-methyl-N-[(1R)-1-{3-[(E)-2-phenylethenyl]phenyl}ethyl]quinazolin-4-amine; 3′-{(1R)-1-[(6,7-dimethoxy-2-methylquinazolin-4-yl)amino]ethyl}-N,N-dimethylbiphenyl-4-carboxamide; 6,7-dimethoxy-2-methyl-N-[(1R)-1-{3-[(1E)-prop-1-en-1-yl]phenyl}ethyl]quinazolin-4-amine; N-{(1R)-1-[3-(cyclopent-1-en-1-yl)phenyl]ethyl}-6,7-dimethoxy-2-methylquinazolin-4-amine; N-(3′-{(1R)-1-[(6,7-dimethoxy-2-methylquinazolin-4-yl)amino]ethyl}biphenyl-3-yl)methanesulfonamide; N-(3′-{(1R)-1-[(6,7-dimethoxy-2-methylquinazolin-4-yl)amino]ethyl}biphenyl-2-yl)acetamide; 6,7-dimethoxy-2-methyl-N-{(1R)-1-[2′-(methylsulfonyl)biphenyl-3-yl]ethyl}quinazolin-4-amine; N-(3′-{(1R)-1-[(6,7-dimethoxy-2-methylquinazolin-4-yl)amino]ethyl}biphenyl-2-yl)methanesulfonamide; N-{1-[5-(3,5-dichlorophenyl)thiophen-2-yl]ethyl}-6,7-dimethoxy-2-methylquinazolin-4-amine; N-{(1R)-1-[3′-(benzyloxy)biphenyl-3-yl]ethyl}-6,7-dimethoxy-2-methylquinazolin-4-amine; N-[(1R)-1-(3′,5′-dichlorobiphenyl-3-yl)ethyl]-6,7-dimethoxy-2-methylquinazolin-4-amine; N-{(1R)-1-[3-(2,3-dihydro-1,4-benzodioxin-6-yl)phenyl]ethyl}-6,7-dimethoxy-2-methylquinazolin-4-anine; 6,7-dimethoxy-2-methyl-N-[1-{5-[3-(5-methyl-1,3,4-oxadiazol-2-yl)phenyl]thiophen-2-yl}ethyl]quinazolin-4-amine; 6,7-dimethoxy-2-methyl-N-[1-{5-[5-(methylsulfonyl)pyridin-3-yl]thiophen-2-yl}ethyl]quinazolin-4-amine; 6,7-dimethoxy-2-methyl-N-[1-{5-[4-(5-methyl-1,3,4-oxadiazol-2-yl)phenyl]thiophen-2-yl}ethyl]quinazolin-4-amine; 6,7-dimethoxy-2-methyl-N-{1-[5-(1H-pyrrolo[2,3-b]pyridin-5-yl)thiophen-2-yl]ethyl}quinazolin-4-amine; 3-(5-{1-[(6,7-dimethoxy-2-methylquinazolin-4-yl)amino]ethyl}thiophen-2-yl)benzenesulfonamide; N-{1-[5-(2-aminopyrimidin-5-yl)thiophen-2-yl]ethyl}-6,7-dimethoxy-2-methylquinazolin-4-amine; N-[1-{5-[(E)-2-cyclopropylethynyl]thiophen-2-yl}ethyl]-6,7-dimethoxy-2-methylquinazolin-4-amine; N-[1-{5-[2-(ethoxymethyl)phenyl]thiophen-2-yl}ethyl]-6,7-dimethoxy-2-methylquinazolin-4-anine; N-{1-[5-(3-fluoro-5-methoxyphenyl)thiophen-2-yl]ethyl}-6,7-dimethoxy-2-methylquinazolin-4-anine; N-[1-{5-[3-(benzyloxy)phenyl]thiophen-2-yl}ethyl]-6,7-dimethoxy-2-methylquinazolin-4-amine; N-{1-[5-(2-butoxy-6-fluorophenyl)thiophen-2-yl]ethyl}-6,7-dimethoxy-2-methylquinazolin-4-amine; 2-[4-(5-{1-[(6,7-dimethoxy-2-methylquinazolin-4-yl)amino]ethyl}thiophen-2-yl)phenyl]-2-methylpropanenitrile; N-[4-(5-{1-[(6,7-dimethoxy-2-methylquinazolin-4-yl)amino]ethyl}thiophen-2-yl)benzyl]acetamide; N-[3-(5-{1-[(6,7-dimethoxy-2-methylquinazolin-4-yl)amino]ethyl}thiophen-2-yl)benzyl]methanesulfonamide; N-[4-(5-{1-[(6,7-dimethoxy-2-methylquinazolin-4-yl)amino]ethyl}thiophen-2-yl)benzyl]methanesulfonamide; 4-(5-{1-[(6,7-dimethoxy-2-methylquinazolin-4-yl)amino]ethyl}thiophen-2-yl)-N-propylbenzamide; 4-(5-{1-[(6,7-dimethoxy-2-methylquinazolin-4-yl)amino]ethyl}thiophen-2-yl)-N-[2-(dimethylamino)ethyl]benzamide; N-[1-{5- {[(2E)-but-2-en-2-yl]thiophen-2-yl}ethyl]-6,7-dimethoxy-2-methylquinazolin-4-amine; N-{1-[5-(5-chloro-2-propoxyphenyl)thiophen-2-yl]ethyl}-6,7-dimethoxy-2-methylquinazolin-4-amine; 6,7-dimethoxy-2-methyl-N-[1-{5-[(1E)-3-phenylprop-1-en-1-yl]thiophen-2-yl}ethyl]quinazolin-4-amine; N-{1-[5-(5-amino-2-methylphenyl)thiophen-2-yl]ethyl}-6,7-dimethoxy-2-methylquinazolin-4-amine; N-{I-[5-(3,5-dimethyl-1,2-oxazol-4-yl)thiophen-2-yl]ethyl}-6,7-dimethoxy-2-methylquinazolin-4-amine; 6,7-dimethoxy-2-methyl-N-[1-{5-[2-(methylsulfonyl)phenyl]thiophen-2-yl}ethyl]quinazolin-4-amine; 6,7-dimethoxy-2-methyl-N-[1-{5-[4-(methylsulfonyl)phenyl]thiophen-2-yl}ethyl]quinazolin-4-amine; 6,7-dimethoxy-2-methyl-N-[1-{5-[2-(trifluoromethoxy)phenyl]thiophen-2-yl}ethyl]quinazolin-4-amine; 6,7-dimethoxy-2-methyl-N-[1-{5-[3-(trifluoromethoxy)phenyl]thiophen-2-yl}ethyl]quinazolin-4-amine; N-{I-[5-(1H-indol-5-yl)thiophen-2-yl]ethyl}-6,7-dimethoxy-2-methylquinazolin-4-amine; N-{1-[5-(furan-3-yl)thiophen-2-yl]ethyl}-6,7-dimethoxy-2-methylquinazolin-4-amine; N-{I-[5-(1-benzothiophen-3-yl)thiophen-2-yl]ethyl}-6,7-dimethoxy-2-methylquinazolin-4-amine; 6,7-dimethoxy-2-methyl-N-{1-[5-(1-methyl-1H-indol-2-yl)thiophen-2-yl]ethyl}quinazolin-4-anine; 6,7-dimethoxy-2-methyl-N-[1-{5-[(1E)-pent-1-en-1-yl]thiophen-2-yl}ethyl]quinazolin-4-amine; N-[1-{5-[(E)-2-cyclohexylethenyl]thiophen-2-yl}ethyl]-6,7-dimethoxy-2-methylquinazolin-4-amine; 6,7-dimethoxy-2-methyl-N-{1-[5-(2-phenoxyphenyl)thiophen-2-yl]ethyl}quinazolin-4-amine; tert-butyl [4-(5-{1-[(6,7-dimethoxy-2-methylquinazolin-4-yl)amino]ethyl}thiophen-2-yl)phenyl]carbamate; (2E)-3-[3-(5-{1-[(6,7-dimethoxy-2-methylquinazolin-4-yl)amino]ethyl}thiophen-2-yl)phenyl]prop-2-enenitrile; N-{1-[5-(2,4-dimethylphenyl)thiophen-2-yl]ethyl}-6,7-dimethoxy-2-methylquinazolin-4-amine; 1-(5′-{1-[(6,7-dimethoxy-2-methylquinazolin-4-yl)amino]ethyl}-2,2′-bithiophen-5-y)ethenone; N-{1-[5-(1,3-benzodioxol-5-yl)thiophen-2-yl]ethyl}-6,7-dimethoxy-2-methylquinazolin-4-amine; 6,7-dimethoxy-2-methyl-N-{1-[5-(4-methyl-3,4-dihydro-2H-1,4-benzoxazin-7-yl)thiophen-2-yl]ethyl}quinazolin-4-amine; N-[3-(5-{1-[(6,7-dimethoxy-2-methylquinazolin-4-yl)amino]ethyl}thiophen-2-yl)phenyl]methanesulfonamide; N-[2-(5-{1-[(6,7-dimethoxy-2-methylquinazolin-4-yl)amino]ethyl}thiophen-2-yl)phenyl]acetamide; 6,7-dimethoxy-N-{1-[5-(3-methoxyphenyl)thiophen-2-yl]ethyl}-2-methylquinazolin-4-amine; 6,7-dimethoxy-2-methyl-N-[1-{5-[3-(trifluoromethyl)phenyl]thiophen-2-yl}ethyl]quinazolin-4-amine; 2-(5-{1-[(6,7-dimethoxy-2-methylquinazolin-4-yl)amino]ethyl}thiophen-2-yl)-N,N-dimethylbenzenesulfonamide; N-[1-{5-[3-(cyclopropylmethoxy)phenyl]thiophen-2-yl}ethyl]-6,7-dimethoxy-2-methylquinazolin-4-amine; N-{1-[5-(1H-indol-4-yl)thiophen-2-yl]ethyl}-6,7-dimethoxy-2-methylquinazolin-4-amine; 6,7-dimethoxy-N-{1-[5-(2-methoxypyrimidin-5-yl)thiophen-2-yl]ethyl}-2-methylquinazolin-4-amine; 6,7-dimethoxy-2-methyl-N-[1-{5-[3-(methylsulfonyl)phenyl]thiophen-2-yl}ethyl]quinazolin-4-amine; N-{1-[5-(2,3-dihydro-1-benzofuran-5-yl)thiophen-2-yl]ethyl}-6,7-dimethoxy-2-methylquinazolin-4-amine; 6,7-dimethoxy-2-methyl-N-[1-{5-[(E)-2-phenylethenyl]thiophen-2-yl}ethyl]quinazolin-4-amine; 4-(5-{1-[(6,7-dimethoxy-2-methylquinazolin-4-yl)amino]ethyl}thiophen-2-yl)-N,N-dimethylbenzamide; 6,7-dimethoxy-2-methyl-N-[1-{5-[(1E)-prop-1-en-1-yl]thiophen-2-yl}ethyl]quinazolin-4-amine; methyl 2-(5-{1-[(6,7-dimethoxy-2-methylquinazolin-4-yl)amino]ethyl}thiophen-2-yl)benzoate; 1-[2-(5-{1-[(6,7-dimethoxy-2-methylquinazolin-4-yl)amino]ethyl}thiophen-2-yl)benzyl]piperidine-4-carboxamide; 2-[2-(5-{1-[(6,7-dimethoxy-2-methylquinazolin-4-yl)amino]ethyl}thiophen-2-yl)phenyl]ethanol; 2-[3-(5-{1-[(6,7-dimethoxy-2-methylquinazolin-4-yl)amino]ethyl}thiophen-2-yl)phenyl]ethanol; 6,7-dimethoxy-2-methyl-N-[1-{5-[2-(2-oxa-6-azaspiro[3.3]hept-6-ylmethyl)phenyl]thiophen-2-yl}ethyl]quinazolin-4-amine; N-[1-(5-bromo-4-methylthiophen-2-yl)ethyl]-6,7-dimethoxy-2-methylquinazolin-4-amine; 6,7-dimethoxy-2-methyl-N-[1-{5-[2-(pyrrolidin-1-ylmethyl)phenyl]thiophen-2-yl}ethyl]quinazolin-4-amine; N-[1-{5-[2-({2-[(dimethylamino)methyl]pyrrolidin-1-yl}methyl)phenyl]thiopent-2-yl}ethyl]-6,7-dimethoxy-2-methylquinazolin-4-amine; 2-methyl-N-[(1R)-]-(naphthalen-1l-yl)ethyl]quinazolin-4-amine; N-[(1R)-1-(4-bromophenyl)ethyl]-6,7-dimethoxy-2-methylquinazolin-4-amine; 6,7-dimethoxy-2-methyl-N-{(1R)-1-[4-(methylsulfonyl)phenyl]ethyl}quinazolin-4-amine; 4-{(1R)-1-[(6,7-dimethoxy-2-methylquinazolin-4-yl)amino]ethyl}benzonitrile; 6,7-dimethoxy-2-methyl-N-[(1R)-1-(3-methylphenyl)ethyl]quinazolin-4-amine; N-[(1R)-1-(3-bromophenyl)ethyl]-6,7-dimethoxy-2-methylquinazolin-4-amine; 4-{(1R)-1-[(6,7-dimethoxy-2-methylquinazolin-4-yl)amino]ethyl}benzamide; N-[(]R)-1-(biphenyl-3-yl)ethyl]-6,7-dimethoxy-2-methylquinazolin-4-amine; 3-{(1R)-1-[(6,7-dimethoxy-2-methylquinazolin-4-y)amino]ethyl}benzonitrile; 6,7-dimethoxy-2-methyl-N-[(1R)-1-(4-methylphenyl)ethyl]quinazolin-4-amine; N-[(1R)-1-(biphenyl-4-yl)ethyl]-6,7-dimethoxy-2-methylquinazolin-4-amine; N-[(1R)-1-(4-cyclopropylphenyl)ethyl]-6,7-dimethoxy-2-methylquinazolin-4-amine; 6,7-dimethoxy-2-methyl-N-{(1R)-1-[3-(methylsulfonyl)phenyl]ethyl}quinazolin-4-amine; 3-{(1R)-1-[(6,7-dimethoxy-2-methylquinazolin-4-yl)amino]ethyl}benzamide; 6,7-dimethoxy-2-methyl-N-{(1R)-1-[3-(1H-pyrazol-4-yl)phenyl]ethyl}quinazolin-4-amine; 6,7-dimethoxy-2-methyl-N-{(1R)-1-[4-(1-methyl-1,2,3,6-tetrahydropyridin-4-yl)phenyl]ethyl}quinazolin-4-amine; 6,7-dimethoxy-2-methyl-N-{(1R)-1-[3-(1-methyl-1,2,3,6-tetrahydropyridin-4-yl)phenyl]ethyl}quinazolin-4-amine; 6,7-dimethoxy-2-methyl-N-{(1R)-1-[4-(prop-1-en-2-yl)phenyl]ethyl}quinazolin-4-amine; N-[(1R)-1-(3-cyclopropylphenyl)ethyl]-6,7-dimethoxy-2-methylquinazolin-4-amine; N-[1-(1-benzothiophen-4-yl)ethyl]-6,7-dimethoxy-2-methylquinazolin-4-amine; 6,7-dimethoxy-2-methyl-N-[(1R)-1-phenylethyl]quinazolin-4-amine 6,7-dimethoxy-2-methyl-N-[1-(thiophen-2-yl)ethyl]quinazolin-4-amine N-[1-(5-bromofuran-2-yl)ethyl]-6,7-dimethoxy-2-methylquinazolin-4-amine; N-[l-(5-bromothiophen-2-yl)ethyl]-6,7-dimethoxy-2-methylquinazolin-4-amine; 1-[2-(5-{1-[(6,7-dimethoxy-2-methylquinazolin-4-yl)amino]ethyl}thiophen-2-yl)benzyl]pyrrolidin-3-ol; N-{1-[5-(2-{[(3S)-3-fluoropyrrolidin-1-yl]methyl}phenyl)thiophen-2-yl]ethyl}-6,7-dimethoxy-2-methylquinazolin-4-amine; 6,7-dimethoxy-2-methyl-N-[1-(quinolin-5-yl)ethyl]quinazolin-4-amine; 6,7-dimethoxy-2-methyl-N-[1-(5-phenylfuran-2-yl)ethyl]quinazolin-4-amine; N-[1-(5-bromo-2,3-dihydro-1-benzofuran-7-yl)ethyl]-6,7-dimethoxy-2-methylquinazolin-4-amine; 6,7-dimethoxy-2-methyl-N-[1-(3-phenoxyphenyl)ethyl]quinazolin-4-amine; 6,7-dimethoxy-2-methyl-N-{1-[3-(2H-tetrazol-5-yl)phenyl]ethyl}quinazolin-4-amine; 6,7-dimethoxy-2-methyl-N-[1-(quinolin-8-yl)ethyl]quinazolin-4-amine 2-[4-(5-{1-[(6,7-dimethoxy-2-methylquinazolin-4-yl)amino]ethyl}thiophen-2-yl)-1H-pyrazol-1-yl]ethanol; N-{1-[5-(6,7-dihydro-5H-pyrrolo[1,2-a]imidazol-3-yl)thiophen-2-yl]ethyl}-6,7-dimethoxy-2-methylquinazolin-4-amine; 6,7-dimethoxy-2-methyl-N-[1-(5-{1-[2-(pyrrolidin-1-yl)ethyl]-1H-pyrazol-4-yl}thiophen-2-yl)ethyl]quinazolin-4-amine; N-{1-[5-(1-cyclopentyl-1H-pyrazol-4-yl)thiophen-2-yl]ethyl}-6,7-dimethoxy-2-methylquinazolin-4-amine; 6,7-dimethoxy-2-methyl-N-{1-[5-(1H-pyrazol-3-yl)thiophen-2-yl]ethyl}quinazolin-4-amine; N-[1-(5-{2-[(3,3-difluoropyrrolidin-1-yl)methyl]phenyl}thiophen-2-yl)ethyl]-6,7-dimethoxy-2-methylquinazolin-4-amine; 6,7-dimethoxy-2-methyl-N-[1-(5-phenylfuran-2-yl)ethyl]quinazolin-4-amine; N-[1-(5-bromo-2,3-dihydro-1-benzofuran-7-yl)ethyl]-6,7-dimethoxy-2-methylquinazolin-4-amine; 2-[1-(5-{1-[(6,7-dimethoxy-2-methylquinazolin-4-yl)amino]ethyl}furan-2-y)-1H-pyrazol-3-yl]ethanol; 5-{1-[(6,7-dimethoxy-2-methylquinazolin-4-yl)amino]ethyl}pyridin-2(1H)-one; 6,7-dimethoxy-2-methyl-N-[1-(3-phenoxyphenyl)ethyl]quinazolin-4-amine; N-[1-(2,1,3-benzothiadiazol-5-yl)ethyl]-6,7-dimethoxy-2-methylquinazolin-4-amine; 6,7-dimethoxy-2-methyl-N-[(1R)-1-(quinolin-8-yl)ethyl]quinazolin-4-amine; N-{1-[5-(cyclopent-1-en-1-yl)thiophen-2-yl]ethyl}-6,7-dimethoxy-2-methylquinazolin-4-amine; N-{1-[5-(2-ethoxyphenyl)thiophen-2-y]ethyl}-6,7-dimethoxy-2-methylquinazolin-4-amine; N-{1-[5-(4-fluoronaphthalen-1-yl)thiophen-2-y]ethyl}-6,7-dimethoxy-2-methylquinazolin-4-amine; N-[1-{5-[2-(aminomethyl)-4-fluorophenyl]thiophen-2-yl}ethyl]-6,7-dimethoxy-2-methylquinazolin-4-amine; N-{1-[5-(3,6-dihydro-2H-pyran-4-yl)thiophen-2-yl]ethyl}-6,7-dimethoxy-2-methylquinazolin-4-amine; tert-butyl {[5-(5-{1-[(6,7-dimethoxy-2-methylquinazolin-4-yl)amino]ethyl}thiophen-2-yl)furan-2-yl]methyl}carbamate; methyl 3-{5-{1-[(6,7-dimethoxy-2-methylquinazolin-4-yl)amino]ethyl}thiophen-2-yl)-1-methyl-1H4-pyrazole-5-carboxylate; N-{1-[5-(2-{[3-(dimethylamino)pyrrolidin-1-yl]methyl}phenyl)thiophen-2-yl]ethyl}-6,7-dimethoxy-2-methylquinazolin-4-amine; N-[1-(5-bromothiophen-3-yl)ethyl]-6,7-dimethoxy-2-methylquinazolin-4-amine; 2-(5-{l-[(6,7-dimethoxy-2-methylquinazolin-4-yl)amino]ethyl}thiophen-2-yl)benzamide; 3-(5-{1-[(6,7-dimethoxy-2-methylquinazolin-4-yl)amino]ethyl}thiophen-2-yl)benzamide; 4-(5-{1-[(6,7-dimethoxy-2-methylquinazolin-4-yl)amino]ethyl}thiophen-2-yl)benzamide; N-{l-[5-(2-aminophenyl)thiophen-2-yl]ethyl}-6,7-dimethoxy-2-methylquinazolin-4-amine; [2-(5-{1-[(6,7-dimethoxy-2-methylquinazolin-4-yl)amino]ethyl}thiophen-2-yl)phenyl]methanol; 2-(5-{1-[(6,7-dimethoxy-2-methylquinazolin-4-yl)amino]ethyl}thiophen-2-yl)benzonitrile; N-{1-[5-(1H-indazol-7-yl)thiophen-2-yl]ethyl}-6,7-dimethoxy-2-methylquinazolin-4-amine; N-{1-[5-(11H-indazol-4-yl)thiophen-2-yl]ethyl}-6,7-dimethoxy-2-methylquinazoline-4-amine; N-{1-[5-(2-ethenylphenyl)thiophen-2-yl]ethyl}-6,7-dimethoxy-2-methylquinazolin-4-amine; 2-[4-(5-{1-[(6,7-dimethoxy-2-methylquinazolin-4-yl)amino]ethyl}thiophen-2-yl)-1H-pyrazol-1-yl]acetamide; 6,7-dimethoxy-2-methyl-N-[1-(5-{2-[(methylamino)methyl]phenyl}thiophen-2-yl)ethyl]quinazolin-4-amine 2-[4-(4-{1-[(6,7-dimethoxy-2-methylquinazolin-4-yl)amino]ethyl}thiophen-2-yl)-1-H-pyrazol-1-yl]ethanol; N-[1-{5-[2-(aminomethyl)phenyl]thiophen-2-yl}ethyl]-6,7-dimethoxy-2-methylquinazolin-4-amine; N-{1-[5-(6,7-dihydro-5H-pyrrolo[1,2-a]imidazol-3-yl)thiophen-3-yl]ethyl}-6,7-dimethoxy-2-methylquinazolin-4-amine; 6,7-dimethoxy-2-methyl-N-[1-(5-{1-[2-(pyrrolidin-1-yl)ethyl]-1H-pyrazol-4-yl}thiophen-3-yl)ethyl]quinazolin-4-amine; N-[1-{5-[2-(aminomethyl)-4-fluorphenyl]thiophen-3-yl}ethyl]-6,7-dimethoxy-2-methylquinazolin-4-amine; 2-[4-(3-{(1R)-1-[(6,7-dimethoxy-2-methylquinazolin-4-yl)amino]ethyl}phenyl)-1H-pyrazol-1-yl]ethanol; 6,7-dimethoxy-2-methyl-N-[(1R)-1-(3-{1-[2-(pyrrolidin-1-yl)ethyl]-1H-pyrazol-4-y}phenyl)ethyl]quinazolin-4-amine; N-{(1R)-1-[2′-(aminomethyl)biphenyl-3-yl]ethyl}-6,7-dimethoxy-2-methylquinazolin-4-amine; N-[1-{5-[2-(aminomethyl)phenyl]thiophen-3-yl}ethyl]-6,7-dimethoxy-2-methylquinazolin-4-amine; N-{(1R)-1-[3-(aminomethyl)phenyl]ethyl}-6,7-dimethoxy-2-methylquinazolin-4-amine; N-{(1R)-1-[3-(6,7-dihydro-5H-pyrrolo[1,2-a]imidazol-3-yl)phenyl]ethyl}-6,7-dimethoxy-2-methylquinazolin-4-amine; N-[1-(5-{2-[(dimethylamino)methyl]phenyl}thiophen-2-yl)ethyl]-6,7-dimethoxy-2-methylquinazolin-4-amine; 6,7-dimethoxy-2-methyl-N-[1-(5-{2-[(methylamino)methyl]phenyl}thiophen-3-yl)ethyl]quinazolin-4-amine; N-[1-(4-bromothiophen-2-yl)ethyl]-6,7-dimethoxy-2-methylquinazolin-4-amine; N-[1-{5-[3-(aminomethyl)phenyl]thiophen-2-yl}ethyl]-6,7-dimethoxy-2-methylquinazolin-4-amine; N-[1-{5-[4-(aminomethyl)phenyl]thiophen-2-yl}ethyl]-6,7-dimethoxy-2-methylquinazolin-4-amine; 6,7-dimethoxy-2-methyl-N-[1-(4-{1-[2-(pyrrolidin-1-yl)ethyl]-1H-pyrazol-3-yl}thiophen-2-yl)ethyl]quinazolin-4-amine; 6,7-dimethoxy-2-methyl-N-[(1R)-1-{2′-[(methylamino)methyl]biphenyl-3-yl}ethyl]quinazolin-4-amine; N-[1-{4-[2-(aminomethyl)-4-fluorophenyl]thiophen-2-yl}ethyl]-6,7-dimethoxy-2-methylquinazolin-4-amine; N-[1-{4-[2-(aminomethyl)phenyl]thiophen-2-yl}ethyl]-6,7-dimethoxy-2-methylquinazolin-4-amine; 6,7-dimethoxy-2-methyl-N-[1-(4-{2-[(methylamino)methyl]phenyl}thiophen-2-yl)ethyl]quinazolin-4-amine; N-{1-[4-(6,7-dihydro-5H-pyrrolo[1,2-a]imidazol-3-yl)thiophen-2-yl]ethyl}-6,7-dimethoxy-2-methylquinazolin-4-amine; 2-[3-(5-{1-[(6,7-dimethoxy-2-methylquinazolin-4-yl)amino]ethyl}thiophen-3-yl)-1H-pyrazol-1-yl]ethanol; N-{(1R)-1-[2′-(aminomethyl)-4′-fluorobiphenyl-3-yl]ethyl}-6,7-dimethoxy-2-methylquinazolin-4-anine; N-[1-{5-[5-(aminomethyl)furan-2-yl]thiophen-2-yl}ethyl]-6,7-dimethoxy-2-methylquinazolin-4-amine; N-{1-[5′-(aminomethyl)-2,2′-bithiophen-5-yl]ethyl}-6,7-dimethoxy-2-methylquinazolin-4-amine; 2-{[2-(5-{1-[(6,7-dimethoxy-2-methylquinazolin-4-yl)amino]ethyl}thiophen-2-yl)benzyl]amino}-1-(1H-indol-3-yl)ethenone; 3-amino-4-(5-{1-[(6,7-dimethoxy-2-methylquinazolin-4-yl)amino]ethyl}thiophen-2-y)-1-benzothiophene-2-carboxamide; 2-[2-(5-{1-[(6,7-dimethoxy-2-methylquinazolin-4-yl)amino]ethyl}thiophen-2-yl)benzyl]glycinamide; 2-[2-(5-{1-[(6,7-dimethoxy-2-methylquinazolin-4-yl)amino]ethyl}thiophen-2-yl)benzyl]-N,N-dimethylglycinamide methyl N-[2-(5-{1-[(6,7-dimethoxy-2-methylquinazolin-4-yl)amino]ethyl}thiophen-2-yl)benzyl]glycinate; 2-[2-(5-{1-[(6,7-dimethoxy-2-methylquinazolin-4-yl)amino]ethyl}thiophen-2-yl)benzyl]-N-methylglycinamide; 2-[2-(5-{1-[(6,7-dimethoxy-2-methylquinazolin-4-yl)amino]ethyl}thiophen-2-yl)benzyl]-N-(2-methoxyethyl)glycinamide; N-benzyl-2-[2-(5-{1-[(6,7-dimethoxy-2-methylquinazolin-4-yl)amino]ethyl}thiophen-2-yl)benzyl]glycinamide; 2-{[2-(5-{1-[(6,7-dimethoxy-2-methylquinazolin-4-yl)amino]ethyl}thiophen-2-yl)benzyl]amino}-1-(morpholin-4-yl)ethenone; 3-(5-{1-[(6,7-dimethoxy-2-methylquinazolin-4-yl)amino]ethyl}thiophen-2-yl)-1,5-dimethyl-1H-pyrrole-2-carbonitrile; 5-{1-[(6,7-dimethoxy-2-methylquinazolin-4-yl)amino]ethyl}-2,3′-bithiophene-4′-carbonitrile; N-[1-(5-{2-[(diethylamino)methyl]phenyl}thiophen-2-yl)ethyl]-6,7-dimethoxy-2-methylquinazolin-4-amine; 2-[2-(5-{1-[(6,7-dimethoxy-2-methylquinazolin-4-yl)amino]ethyl}thiophen-2-yl)benzyl]-N-phenylglycinamide; 1-[2-(5-{1-[(6,7-dimethoxy-2-methylquinazolin-4-yl)amino]ethyl}thiophen-2-yl)benzyl]piperidine-3-carboxamide; N-{1-[5-(2-{[(2,2-difluoroethyl)(methyl)amino]methyl}phenyl)thiophen-2-yl]ethyl}-6,7-dimethoxy-2-methylquinazolin-4-amine; N-[1-{5-[2-(aminomethyl)-5-chlorophenyl]thiophen-2-yl}ethyl]-6,7-dimethoxy-2-methylquinazolin-4-amine; N-[2-(5-{1-[(6,7-dimethoxy-2-methylquinazolin-4-yl)amino]ethyl}thiophen-2-yl)-5-fluorobenzyl]-1H-imidazole-2-carboxamide; N-[2-(5-{1-[(6,7-dimethoxy-2-methylquinazolin-4-yl)amino]ethyl}thiophen-2-yl)-5-fluorobenzyl]-1H-imidazole-5-carboxamide; N-[2-(5-{1-[(6,7-dimethoxy-2-methylquinazolin-4-yl)amino]ethyl}thiophen-2-yl)-5-fluorobenzyl]-N&lt;sup&gt;2&lt;/sup&gt;-(2,2,2-trifluoroethyl)glycinamide; N-[2-(5-{1-[(6,7-dimethoxy-2-methylquinazolin-4-yl)amino]ethyl}thiophen-2-yl)-5-fluorobenzyl]-1H-indole-2-carboxamide; 2-{[2-(5-{l-[(6,7-dimethoxy-2-methylquinazolin-4-yl)amino]ethyl}thiophen-2-yl)benzyl]amino}ethanol; 2-{[2-(5-{1-[(6,7-dimethoxy-2-methylquinazolin-4-yl)amino]ethyl}thiophen-2-yl)benzyl](methyl)amino}ethanol; N-[2-(5-{l-[(6,7-dimethoxy-2-methylquinazolin-4-yl)amino]ethyl}thiophen-2-yl)-5-fluorobenzyl]-N2-phenylglycinamide; 6,7-dimethoxy-2-methyl-N-{l-[5-(2-{[(2,2,2-trifluoroethyl)amino]methyl}phenyl)thiophen-2-yl]ethyl}quinazolin-4-amine; 6,7-dimethoxy-2-methyl-N-[1-(5-{2-[(pyridin-2-ylamino)methyl]phenyl}thiophen-2-yl)ethyl]quinazolin-4-amine; 6,7-dimethoxy-2-methyl-N-[1-(5-{2-[(1H-pyrazol-3-ylamino)methyl]phenyl}thiophen-2-yl)ethyl]quinazolin-4-amine 1-(3,4-dihydroisoquinolin-2(1H)-yl)-2-{[2-(5-{1-[(6,7-dimethoxy-2-methylquinazolin-4-yl)amino]ethyl}thiophen-2-yl)benzyl]amino}ethano; N-[1-{5-[4-fluoro-2-({[(1-methyl-1H-imidazol-2-yl)methyl]amino}methyl)phenyl]thiophen-2-yl}ethyl]-6,7-dimethoxy-2-methylquinazolin-4-amine; 6,7-dimethoxy-2-methyl-N-[1-{5-[2-(piperazin-1-ylmethyl)phenyl]thiophen-2-yl}ethyl]quinazolin-4-amine hydrochloride tert-butyl 4-[2-(5-{1-[(6,7-dimethoxy-2-methylquinazolin-4-yl)amino]ethyl}thiophen-2-yl)benzyl]piperazine-1-carboxylate; N-[2-(5-{1-[(6,7-dimethoxy-2-methylquinazolin-4-yl)amino]ethyl}thiophen-2-yl)-5-fluorobenzyl]acetamide; 6,7-dimethoxy-2-methyl-N-[1-(5-{2-[(4-methylpiperazin-1-yl)methyl]phenyl}thiophen-2-yl)ethyl]quinazolin-4-amine; (3S)-{[2-(5-{1-[(6,7-dimethoxy-2-methylquinazolin-4-yl)amino]ethyl}thiophen-2-yl)benzyl]amino}-1-methylpyrrolidin-2-one; N-[2-(5-{1-[(6,7-dimethoxy-2-methylquinazolin-4-yl)amino]ethyl}thiophen-2-yl)-5-fluorobenzyl]-1H-pyrazole-3-carboxamide; 6,7-dimethoxy-2-methyl-N-[1-{5-[2-(morpholin-4-ylmethyl)phenyl]thiophen-2-yl}ethyl]quinazolin-4-amine; 1-[2-(5-{1-[(6,7-dimethoxy-2-methylquinazolin-4-yl)amino]ethyl}thiophen-2-yl)benzyl]azetidin-3-ol; 2-[2-(5-{1-[(6,7-dimethoxy-2-methylquinazolin-4-yl)amino]ethyl}thiophen-2-yl)benzyl]-2,5,7-triazaspiro[3.4]octan-6-one; N-[2-(5-{1-[(6,7-dimethoxy-2-methylquinazolin-4-yl)amino]ethyl}thiophen-2-y)-5-fluorobenzyl]-L-prolinamide; N-{1-[5-(2-{[(2,2-difluoroethyl)amino]methyl}phenyl)thiophen-2-yl]ethyl}-6,7-dimethoxy-2-methylquinazolin-4-amine; N-[2-(5-{1-[(6,7-dimethoxy-2-methylquinazolin-4-yl)amino]ethyl}thiophen-2-yl)-5-fluorobenzyl]-prolinamide; N-[1-{5-[2-(azetidin-1-ylmethyl)phenyl]thiophen-2-yl}ethyl]-6,7-dimethoxy-2-methylquinazolin-4-amine; {1-[(2S)-(5-{1-[(6,7-dimethoxy-2-methylquinazolin-4-yl)amino]ethyl}thiophen-2-yl)benzyl]azetidin-2-yl}methanol; N-{1-[5-(2-{[3-(dimethylamino)azetidin-1-yl]methyl}phenyl)thiophen-2-yl]ethyl}-6,7-dimethoxy-2-methylquinazolin-4-amine; N-[1-(5-{2-[(3,3-difluoroazetidin-1-yl)methyl]phenyl}thiophen-2-yl)ethyl]-6,7-dimethoxy-2-methylquinazolin-4-amine; 6,7-dimethoxy-2-methyl-N-{1-[5-(2-{[methyl(2,2,2-trifluoroethyl)amino]methyl}phenyl)thiophen-2-yl]ethyl}quinazolin-4-amine; N-[1-(5-{2-[(3-fluoroazetidin-1-yl)methyl]phenyl}thiophen-2-yl)ethyl]-6,7-dimethoxy-2-methylquinazolin-4-amine; N-[1-(5-{4-chloro-2-[(dimethylamino)methyl]phenyl}thiophen-2-yl)ethyl]-6,7-dimethoxy-2-methylquinazolin-4-amine; 1-[2-(5-{1-[(6,7-dimethoxy-2-methylquinazolin-4-yl)amino]ethyl}thiophen-2-yl)phenyl]ethenone; 6,7-dimethoxy-2-methyl-N-[1-(5-{2-[2-(pyrrolidin-1-yl)ethoxy]phenyl}thiophen-2-yl)ethyl]quinazolin-4-amine; 6,7-dimethoxy-2-methyl-N-[1-(4-{2-[(methylamino)methyl]phenyl}thiophen-2-yl)ethyl]quinazolin-4-amine, enantiomer 1; 6,7-dimethoxy-2-methyl-N-[1-(4-{2-[(methylamino)methyl]phenyl}thiophen-2-yl)ethyl]quinazolin-4-amine, enantiomer 2; N-{1-[5-(6,7-dihydro-5H-pyrrolo[1,2-a]imidazol-3-yl)thiophen-2-yl]ethyl}-6,7-dimethoxy-2-methylquinazolin-4-amine, enantiomer 1; N-{1-[5-(6,7-dihydro-5H-pyrrolo[1,2-a]imidazol-3-yl)thiophen-2-yl]ethyl}-6,7-dimethoxy-2-methylquinazolin-4-amine, enantiomer 2; 6,7-dimethoxy-2-methyl-N-[1-(2-methyl-1-benzothiophen-4-yl)ethyl]quinazolin-4-amine; 6,7-dimethoxy-2-methyl-N-[1-(thieno[2,3-b]pyridin-4-yl)ethyl]quinazolin-4-amine; 6,7-dimethoxy-2-methyl-N-[1-(thieno[2,3-c]pyridin-4-yl)ethyl]quinazolin-4-amine; 6,7-dimethoxy-2-methyl-N-[1-(thieno[3,2-c]pyridin-4-yl)ethyl]quinazolin-4-amine; N-{(1R)-1-[3-(3,5-dimethyl-1T-pyrazol-4-yl)phenyl]ethyl}-6,7-dimethoxy-2-methylquinazolin-4-amine; 6,7-dimethoxy-2-methyl-N-{(1R)-1-[3-(5-methyl-1T-pyrazol-4-yl)phenyl]ethyl}quinazolin-4-amine; N-((1R)-1-[3-(3,5-dimethyl-1,2-oxazol-4-yl)phenyl]ethyl}-6,7-dimethoxy-2-methylquinazolin-4-amine; 6,7-dimethoxy-2-methyl-N-{(1R)-1-[3-(1H-pyrazol-5-yl)phenyl]ethyl}quinazolin-4-amine; 6,7-dimethoxy-2-methyl-N-{(1R)-1-[3-(1-methyl-1H-pyrazol-4-yl)phenyl]ethyl}quinazolin-4-amine; 6,7-dimethoxy-2-methyl-N-{(1R)-1-[3-(1-methyl-1H-pyrazol-5-yl)phenyl]ethyl}quinazolin-4-amine; N-{(1R)-1-[3-(1H-imidazol-1-yl)phenyl]ethyl}-6,7-dimethoxy-2-methylquinazolin-4-amine; 6,7-dimethoxy-2-methyl-N-{(1R)-1-[3-(1H-pyrazol-1-yl)phenyl]ethyl}quinazolin-4-amine; N-{(1R)-1-[3-(l H-imidazol-4-yl)phenyl]ethyl}-6,7-dimethoxy-2-methylquinazolin-4-amine; 6-(benzyloxy)-N-[(1R)-1-(3-bromophenyl)ethyl]-7-methoxy-2-methylquinazolin-4-amine; 6-(benzyloxy)-7-methoxy-2-methyl-N-{(1R)-1-[3-(1H-pyrazol-4-yl)phenyl]ethyl}quinazolin-4-amine; 7-methoxy-2-methyl-4-({(1R)-1-[3-(1H-pyrazol-4-yl)phenyl]ethyl}amino)quinazolin-6-ol; 6-(cyclopropylmethoxy)-7-methoxy-2-methyl-N-{(1R)-1-[3-(1H-pyrazol-4-yl)phenyl]ethyl}quinazolin-4-amine; 6,7-dimethoxy-2-methyl-N-[1-(thiophen-2-yl)ethyl]quinazolin-4-amine; 7-methoxy-6-(2-methoxyethoxy)-2-methyl-N-{(1R)-1-[3-(1H-pyrazol-4-yl)phenyl]ethyl}quinazolin-4-amine (1R)-1-[2-(5-{1-[(6,7-dimethoxy-2-methylquinazolin-4-yl)amino]ethyl}thiophen-2-yl)phenyl]propan-1-ol; 6-butoxy-7-methoxy-2-methyl-N-{(1R)-1-[3-(1H-pyrazol-4-yl)phenyl]ethyl}quinazolin-4-amine; 7-methoxy-2-methyl-6-(3-methylbutoxy)-N-{(1R)-1-[3-(1H-pyrazol-4-yl)phenyl]ethyl}quinazolin-4-amine; tert-butyl {2-[2-(5-{1-[(6,7-dimethoxy-2-methylquinazolin-4-yl)amino]ethyl}thiophen-2-yl)phenyl]ethyl}carbamate; 7-methoxy-2-methyl-6-(propan-2-yloxy)-N-{(1R)-1-[3-(1H-pyrazol-4-yl)phenyl]ethyl}quinazolin-4-amine; 7-methoxy-2-methyl-6-(oxetan-3-ylmethoxy)-N-{(1R)-1-[3-(1H-pyrazol-4-yl)phenyl]ethyl}quinazolin-4-amine; 6-ethoxy-7-methoxy-2-methyl-N-{(1R)-1-[3-(1H-pyrazol-4-yl)phenyl]ethyl}quinazolin-4-amine; 6-ethoxy-N-{(1R)-1-[3-(1-ethyl-1H-pyrazol-4-yl)phenyl]ethyl}-7-methoxy-2-methylquinazolin-4-amine; N-[1-{5-[2-(2-aminoethyl)phenyl]thiophen-2-yl}ethyl]-6,7-dimethoxy-2-methylquinazolin-4-amine; tert-butyl {1-[2-(5-{1-[(6,7-dimethoxy-2-methylquinazolin-4-yl)amino]ethyl}thiophen-2-yl)phenyl]ethyl}carbamate; N-[1-(5-{2-[1-aminoethyl]phenyl}thiophen-2-yl)ethyl]-6,7-dimethoxy-2-methylquinazolin-4-amine; N-[1-(5-{2-[1-aminoethyl]phenyl}thiophen-2-yl)ethyl]-6,7-dimethoxy-2-methylquinazolin-4-amine; 6,7-dimethoxy-2-methyl-N-[1-{5-[2-(1H1-pyrazol-4-yl)phenyl]thiophen-2-yl}ethyl]quinazolin-4-amine; 6,7-dimethoxy-2-methyl-N-[1-(5-{2-[(phenylamino)methyl]phenyl}thiophen-2-yl)ethyl]quinazolin-4-amine 6-bromo-N-[(1R)-1-(3-chlorophenyl)ethyl]-2-methylquinazolin-4-amine N-[1-(5-{2-[(cyclopentylamino)methyl]phenyl}thiophen-2-yl)ethyl]-6,7-dimethoxy-2-methylquinazolin-4-amine; N-[1-(5-{2-[(benzylamino)methyl]phenyl}thiophen-2-yl)ethyl]-6,7-dimethoxy-2-methylquinazolin-4-amine; N-[1-(5-{2-[(butylamino)methyl]phenyl}thiophen-2-yl)ethyl]-6,7-dimethoxy-2-methylquinazolin-4-amine; N-[1-(5-{2-[(ethylamino)methyl]phenyl}thiophen-2-yl)ethyl]-6,7-dimethoxy-2-methylquinazolin-4-amine; 6,7-dimethoxy-2-methyl-N-[1-{5-[2-(1H-tetrazol-5-yl)phenyl]thiophen-2-yl}ethyl]quinazolin-4-amine; 6,7-dimethoxy-N-{1-[5-(2-{[(2-methoxyethyl)amino]methyl}phenyl)thiophen-2-yl]ethyl}-2-methylquinazolin-4-amine; N-[1-(5-{2-[(cyclopropylamino)methyl]phenyl}thiophen-2-yl)ethyl]-6,7-dimethoxy-2-methylquinazolin-4-amine; methyl 4-{[(1R)-1-(3-chlorophenyl)ethyl]amino}-2-methylquinazoline-6-carboxylate; 4-{[(1R)-1-(3-chlorophenyl)ethyl]amino}-2-methylquinazoline-6-carboxylic acid; (4-{[(1R)-1-(3-chlorophenyl)ethyl]amino}-2-methylquinazolin-6-yl)methanol; [2-(5-{1-[(6,7-dimethoxy-2-methylquinazolin-4-yl)amino]ethyl}thiophen-2-yl)phenyl](phenyl)methanol; 1-[2-(5-{1-[(6,7-dimethoxy-2-methylquinazolin-4-yl)amino]ethyl}thiophen-2-yl)phenyl]-3-phenylpropan-1-ol; 1-[2-(5-{1-[(6,7-dimethoxy-2-methylquinazolin-4-yl)amino]ethyl}thiophen-2-yl)phenyl]-2-phenylethanol; 1-[2-(5-{1-[(6,7-dimethoxy-2-methylquinazolin-4-yl)amino]ethyl}thiophen-2-yl)phenyl]pentan-1-ol; 1-[2-(5-{1-[(6,7-dimethoxy-2-methylquinazolin-4-yl)amino]ethyl}thiophen-2-yl)phenyl]prop-2-yn-1-ol; 1-[2-(5-{1-[(6,7-dimethoxy-2-methylquinazolin-4-yl)amino]ethyl}thiophen-2-yl)phenyl]-2-methylpropan-1-ol; 1-[2-(5-{1-[(6,7-dimethoxy-2-methylquinazolin-4-yl)amino]ethyl}thiophen-2-yl)phenyl]-2,2,2-trifluoroethanol; N-{1-[5-(6,7-dihydro-5H1-pyrrolo[1,2-a]imidazol-3-yl)-4-methylthiophen-2-Ly]ethyl}-6,7-dimethoxy-2-methylquinazolin-4-anine; N-[2-(5-{l-[(6,7-dimethoxy-2-methylquinazolin-4-yl)amino]ethyl}thiophen-2-y)-5-fluorobenzyl]-2-hydroxyacetamide; N-[2-(5-{1-[(6,7-dimethoxy-2-methylquinazolin-4-yl)amino]ethyl}thiophen-2-y)-5-fluorobenzyl]-2-methoxyacetamide; N-(1-(5-(4-bromo-2-((dimethylamino)methyl)phenyl)thiophen-2-yl)ethyl)-6,7-dimethoxy-2-methylquinazolin-4-amine; N-(1-(5-(2-((Dimethylamino) methyl)-4-(trifluoromethyl) phenyl) thiophen-2-yl)ethyl)-6,7-dimethoxy-2-methylquinazoline-4-anine; 6,7-dimethoxy-2-methyl-N-(1-{5-[2-methyl-4-(trifluoromethyl)phenyl]-2-thienyl}ethyl)quinazolin-4-amine; tert-butyl [4-chloro-2-(5-{I-[(6,7-dimethoxy-2-methylquinazolin-4-yl)amino]ethyl}thiophen-2-yl)benzyl]carbamate; 4-(3-{(1R)-1-[(6,7-dimethoxy-2-methylquinazolin-4-yl)amino]ethyl}phenyl)pyridin-2-ol; N-{l-[3-(benzyloxy)phenyl]ethyl}-6,7-dimethoxy-2-methylquinazolin-4-amine; N-[1-(5-{2-[(dimethylamino)methyl]phenyl}thiophen-2-yl)ethyl]-6,7-dimethoxy-2-methylquinazolin-4-amine (enantiomer 1); N-[1-(5-{2-[(dimethylamino)methyl]phenyl}thiophen-2-yl)ethyl]-6,7-dimethoxy-2-methylquinazolin-4-amine (enantiomer 2); 2-(4-{[(1R)-1-(3-chiorophenyl)ethyl]amino}-2-methylquinazolin-6-yl)propan-2-ol; 2-(3′-{(1R)-1-[(6,7-dimethoxy-2-methylquinazolin-4-yl)amino]ethyl}biphenyl-2-yl)acetamide; 2-[2-(5-{1-[(6,7-dimethoxy-2-methylquinazolin-4-yl)amino]ethyl}thiophen-2-yl)phenyl]acetamide; 5-(5-{1-[(6,7-dimethoxy-2-methylquinazolin-4-yl)amino]ethyl}thiophen-2-yl)pyridin-2-ol; N-[(1R)-1-(3-chlorophenyl)ethyl]-6-methoxy-2,8-dimethylquinazolin-4-amine; N-[1-{5-[2-(aminomethyl)-3-chlorophenyl]thiophen-2-yl}ethyl]-6,7-dimethoxy-2-methylquinazolin-4-amine; N-[1-{5-[2-(aminomethyl)-4-chlorophenyl]thiophen-2-yl}ethyl]-6,7-dimethoxy-2-methylquinazolin-4-amine; N-(1-{5-[2-(aminomethyl)-4-fluorophenyl]-4-methyl-2-thienyl}ethyl)-6,7-dimethoxy-2-methylquinazolin-4-amine; N-[1-(5-{2-[(dimethylamino)methyl]phenyl}-4-methyl-2-thienyl)ethyl]-6,7-dimethoxy-2-methylquinazolin-4-anine; 2-{[2-(5-{1-[(6,7-dimethoxy-2-methylquinazolin-4-yl)amino]ethyl}-3-methyl-2-thienyl)benzyl](methyl)amino}ethanol; 6,7-dimethoxy-2-methyl-N-[1-(4-methyl-5-{2-[(methylamino)methyl]-phenyl}thiophen-2-yl)ethyl]quinazolin-4-amine; 1-[2-(5-{1-[(6,7-dimethoxy-2-methylquinazolin-4-yl)amino]ethyl}thiophen-2-yl)phenyl]ethane-1,2-diol; N-[(1R)-1-(3-chlorophenyl)ethyl]-2,6-dimethylquinazolin-4-amine; N-[(1R)-1-(3-chlorophenyl)ethyl]-2-methyl-6-(1H-pyrazol-4-yl)quinazolin-4-amine; N-[(1R)-1-(3-chlorophenyl)ethyl]-2-methyl-6-(1-methyl-1H-pyrazol-4-yl)quinazolin-4-amine; N-[(1R)-1-(3-chlorophenyl)ethyl]-6-cyclopropyl-2-methylquinazolin-4-amine; tert-butyl [3-chloro-2-(5-{1-[(6,7-dimethoxy-2-methylquinazolin-4-yl)amino]ethyl}thiophen-2-yl)benzyl]carbamate; N-[1-{5-[2-(aminomethyl)-6-chlorophenyl]thiophen-2-yl}ethyl]-6,7-dimethoxy-2-methylquinazolin-4-amine; 4-(5-{1-[(6,7-dimethoxy-2-methylquinazolin-4-yl)amino]ethyl}-2-thienyl)pyridin-2-ol; 4-[2-(5-{1-[(6,7-dimethoxy-2-methylquinazolin-4-yl)amino]ethyl}thiophen-2-yl)phenyl]azetidin-2-one; N-[(1R)-1-(3-chlorophenyl)ethyl]-6-methoxy-2,7-dimethylquinazolin-4-amine; 4-(5-{1-[(6,7-dimethoxy-2-methylquinazolin-4-yl)amino]ethyl}thiophen-2-yl)-3-[(dimethylamino)methyl]benzonitrile; N-[1-(5-bromothiophen-2-yl)ethyl]-6-[3-(dimethylamino)pyrrolidin-1-yl]-2-methylquinazolin-4-amine; N-[1-(5-{2-[(dimethylamino)methyl]phenyl}thiophen-2-yl)ethyl]-2-methyl-6-(pyrrolidin-1-y)quinazolin-4-amine; N-[1-(5-bromothiophen-2-yl)ethyl]-2-methyl-6-(4-methylpiperazin-1-yl)quinazolin-4-amine; N-[1-(5-{2-[(dimethylamino)methyl]phenyl}thiophen-2-yl)ethyl]-2-methyl-6-(4-methylpiperazin-1-yl)quinazolin-4-amine; N-[1-(5-{2-[(dimethylamino)methyl]phenyl}thiophen-2-yl)ethyl]-6-[3-(dimethylamino)pyrrolidin-1-yl]-2-methylquinazolin-4-amine; N-[(1R)-1-(5-bromothiophen-2-yl)ethyl]-2-methyl-6-(pyrrolidin-1-yl)quinazolin-4-amine; N-(4-{[1-(5-{2-[(dimethylamino)methyl]phenyl}thiophen-2-yl)ethyl]amino}-2-methylquinazolin-6-yl)acetamide; N-[1-(5-{2-[(dimethylamino)methyl]phenyl}thiophen-2-yl)ethyl]-2-methyl-6-nitroquinazolin-4-amine; 6,7-dimethoxy-N-{1-[5-(4-methoxy-2-methylphenyl)thiophen-2-yl]ethyl}-2-methylquinazolin-4-amine; N4-[1-(5-{2-[(dimethylamino)methyl]phenyl}-2-thienyl)ethyl]-2-methyl-quinazoline-4,6-diamine; N-[1-(5-bromothiophen-2-yl)ethyl]-2-methyl-6-[4-(pyridin-3-ylmethyl)piperazin-1-yl]quinazolin-4-amine; N4-[1-(5-bromo-2-thienyl)ethyl]-2-methyl-N6-[2-(morpholin-4-y)ethyl]-quinazoline-4,6-diamine; N-[1-(5-{2-[(dimethylamino)methyl]phenyl}thiophen-2-y)ethyl]-2-methyl-6-[4-(pyridin-3-ylmethyl)piperazin-1-yl]quinazolin-4-amine; N-{2-[(4-{[1-(5-{2-[(dimethylamino)methyl]phenyl}thiophen-2-yl)ethyl]amino}-2-methylquinazolin-6-yl)amino]ethyl}acetamide; N-[1-(5-bromo-3-chlorothiophen-2-yl)ethyl]-6,7-dimethoxy-2-methylquinazolin-4-amine; tert-butyl [2-(4-chloro-5-{1-[(6,7-dimethoxy-2-methylquinazolin-4-yl)amino]ethyl}thiophen-2-yl)benzyl]carbamate; N-[1-{5-[2-(aminomethyl)phenyl]-4-chlorothiophen-2-yl}ethyl]-6,7-dimethoxy-2-methylquinazolin-4-amine; N-[l-(4-chloro-5-{2-[(dimethylamino)methyl]phenyl}thiophen-2-yl)ethyl]-6,7-dimethoxy-2-methylquinazolin-4-amine; N-[1-(5-bromo-4-chlorothiophen-2-yl)ethyl]-6,7-dimethoxy-2-methylquinazolin-4-amine; N4-[1-(5-{2-[(dimethylamino)methyl]phenyl}-2-thienyl)ethyl]-2-methyl-N6-[2-(morpholin-4-yl)ethyl]quinazoline-4,6-diamine; 4-(4-{[1-(5-bromothiophen-2-yl)ethyl]amino}-2-methylquinazolin-6-yl)-1-methylpiperazin-2-one; 4-(4-{[1-(5-{2-[(dimethylamino)methyl]phenyl}thiophen-2-yl)ethyl]amino}-2-methylquinazolin-6-yl)-1-methylpiperazin-2-one methyl 2-[2-(5-{1-[(6,7-dimethoxy-2-methylquinazolin-4-yl)amino]ethyl}-thiophen-2-yl)phenyl]-2-methylpropanoate; N-[1-{5-[2-(aminomethyl)phenyl]-3-chlorothiophen-2-yl}ethyl]-6,7-dimethoxy-2-methylquinazolin-4-amine; N-(4-{[1-(5-{2-[(dimethylamino)methyl]phenyl}thiophen-2-yl)ethyl]amino}-2-methylquinazolin-6-yl)methanesulfonamide; N-[1-(5-{2-[(dimethylamino)methyl]-4-methoxyphenyl}thiophen-2-yl)ethyl]-6,7-dimethoxy-2-methylquinazolin-4-amine; 3-(4-{[1-(5-{2-[(dimethylamino)methyl]phenyl}thiophen-2-yl)ethyl]amino}-2-methylquinazolin-6-yl)-1,1-dimethylurea; 1-benzyl-4-(4-{[1-(5-bromothiophen-2-yl)ethyl]amino}-2-methylquinazolin-6-yl)piperazin-2-one; N-[1-(5-{2-[(dimethylamino)methyl]-4-methylphenyl}thiophen-2-yl)ethyl]-6,7-dimethoxy-2-methylquinazolin-4-amine; N-[1-(5-{4-cyclopropyl-2-[(dimethylamino)methyl]phenyl}thiophen-2-yl)ethyl]-6,7-dimethoxy-2-methylquinazolin-4-amine; 6,7-dimethoxy-2-methyl-N-[1-(2-methyl-1,3-thiazol-4-yl)ethyl]quinazolin-4-amine; 6,7-dimethoxy-2-methyl-N-[1-(4-methyl-1,3-thiazol-2-yl)ethyl]quinazolin-4-amine; 3-(5-{I-[(6,7-dimethoxy-2-methylquinazolin-4-yl)amino]ethyl}thiophen-2-yl)-1-methyl-1H-pyrazole-5-carboxylic acid; tert-butyl [(5′-{1-[(6,7-dimethoxy-2-methylquinazolin-4-yl)amino]ethyl}-2,2′-bithiophen-5-yl)methyl]carbamate; 7-methoxy-2-methyl-6-[2-(methylsulfonyl)ethoxy]-N-{(1R)-1-[3-(1H-pyrazol-4-yl)phenyl]ethyl}quinazolin-4-amine; tert-butyl [5-chloro-2-(5-{1-[(6,7-dimethoxy-2-methylquinazolin-4-yl)amino]ethyl}thiophen-2-yl)benzyl]carbamate; tert-butyl [2-chloro-6-(5-{1-[(6,7-dimethoxy-2-methylquinazolin-4-yl)amino]ethyl}thiophen-2-yl)benzyl]carbamate; 7-bromo-N-[(1R)-1-(3-chlorophenyl)ethyl]-2-methylquinazolin-4-amine; N-[1-(5-bromothiophen-2-yl)ethyl]-2-methyl-6-nitroquinazolin-4-amine; methyl 4-{[(1R)-1-(3-chlorophenyl)ethyl]amino}-2-methylquinazoline-7-carboxylate; 3-amino-3-[2-(5-{1-[(6,7-dimethoxy-2-methylquinazolin-4-yl)amino]ethyl}thiophen-3-yl)phenyl]propanoic acid; N-[1-{5-[2-(2-aminopropan-2-yl)phenyl]thiophen-2-yl}ethyl]-6,7-dimethoxy-2-methylquinazolin-4-amine; {[2-(5-{1-[(6,7-dimethoxy-2-methylquinazolin-4-yl)amino]ethyl}thiophen-2-yl)benzyl](methyl)amino}acetonitrile; 1-(4-{[1-(5-{2-[(dimethylamino)methyl]phenyl}thiophen-2-yl)ethyl]amino}-2-methylquinazolin-6-yl)-3-methylurea; 1-benzyl-4-(4-{[1-(5-{2-[(dimethylamino)methyl]phenyl}thiophen-2-yl)ethyl]amino}-2-methylquinazolin-6-yl)piperazin-2-one; or a stereoisomer, a tautomer, an N-oxide, a hydrate, a solvate, or a salt thereof, or a mixture of same.

In some embodiments, the SOS inhibitor has the structure, or a stereoisomer, a tautomer, an N-oxide, a hydrate, a solvate, or a salt thereof, or a mixture of same:

6-ethoxy-N-[(1R)-1-(4-fluorophenyl)ethyI]-2-methylpyrido[3,4-d]pyrimidin-4-amine
N-[(1R)-1-(4-fluorophenyl)ethyI]-6-methoxy-2-methylpyrido[3,4-d]pyrimidin-4-amine
N41-(1-benzothiophen-4-yl)ethyl]-6-ethoxy-2-methylpyrido[3,4-d]pyrimidin-4-amine
N41-(1-benzothiophen-4-yl)ethyl]-6-methoxy-2-methylpyrido[3,4-d]pyrimidin-4-amine
N-[(1R)-1-(3-bromophenyl)ethyI]-6-ethoxy-2-methylpyrido[3,4-d]pyrimidin-4-amine
N-[(1R)-1-(3-chlorophenyl)ethyI]-6-ethoxy-2-methylpyrido[3,4-d]pyrimidin-4-amine
6-ethoxy-2-methyl-N-{(1R)-143-(methylsulfonyl)phenyl]ethyllpyrido[3,4-d]pyrimidin-4-amine
N-[(1R)-1-(3-chloro-4-fluorophenyl)ethyI]-6-methoxy-2-methylpyrido[3,4-d]pyrimidin-4-.
6-fluoro-N-[(1R)-1-(4-fluorophenyl)ethyI]-2-methylpyrido[3,4-d]pyrimidin-4-amine
N-[(1R)-1-(4-fluorophenyl)ethyI]-6-(2-methoxyethoxy)-2-methylpyrido[3,4-d]pyrimidin-4-amine
N-[(1R)-1-(4-fluorophenyl)ethyI]-2-methyl-6-(tetrahydro-2H-pyran-4-ylmethoxy)pyrido[3,4-d]pyrimidin-4-
amine
N-[(1R)-1-(3-cyclopropy1-4-fluorophenyl)ethy1]-6-methoxy-2-methylpyrido[3,4-d]pyrimidin-4-
amine
N-[(1R)-1-(4-bromophenyl)ethyI]-6-ethoxy-2-methylpyrido[3,4-d]pyrimidin-4-amine
6-methoxy-2-methyl-N-[1-(1-methyl-1H-indazol-4-
yl)ethyl]pyrido[3,4-d]pyrimidin-4-amine
N-[(1R)-1-(4-fluorophenyl)ethyI]-2-methyl-6-(propan-2-yloxy)pyrido[3,4-d]pyrimidin-4-amine
N-[(1R)-1-(4-fluorophenyl)ethyI]-2-methyl-6-
(methylsulfanyl)pyrido[3,4-d]pyrimidin-4-amine
6-methoxy-2-methyl-N-[1-(2-methyl-2H-indazol-4-
yl)ethyl]pyrido[3,4-d]pyrimidin-4-amine
4-{(1R)-1-[(6-ethoxy-2-methylpyrido[3,4-d]pyrimidin-4-yl)amino]ethyllbenzonitrile
6-ethoxy-2-methyl-N-[(1R)-1-phenylethyl]pyrido[3,4-d]pyrimidin-4-amine
6-(benzyloxy)-N-[(1R)-1-(3-bromophenyl)ethyI]-2-
methylpyrimido[5,4-d]pyrimidin-4-amine
N8-[(1R)-1-(3-bromophenyl)ethyI]-N2,N2,6-trimethylpyrimido[5,4-d]pyrimidine-2,8-diamine
N-[(1R)-1-(3-bromophenyl)ethyI]-2-methyl-6-(morpholin-4-yl)pyrimido[5,4-d]pyrimidin-4-amine
N-[(1R)-1-(3-bromophenyl)ethyI]-6-ethoxy-2-methylpyrimido[5,4-cl]pyrimidin-4-amine
N-[(1R)-1-(3-bromophenyl)ethyI]-6-methoxy-2-methylpyrimido[5,4-cl]pyrimidin-4-amine
N-[(1R)-1-(3-bromophenyl)ethyI]-2-methyl-6-phenoxypyrimido[5,4-cl]pyrimidin-4-amine
N-[(1R)-1-(3-bromophenyl)ethyI]-6-(2-methoxyethoxy)-2-methylpyrimido[5,4-cl]pyrimidin-4-
amine
N-[(1R)-1-(3-bromophenyl)ethyI]-2-methyl-6-(morpholin-4-yl)pyrido[3,2-cl]pyrimidin-4-amine
N-[(1R)-1-(3-bromophenyl)ethyI]-6-methoxy-2-methylpyrido[3,2-cl]pyrimidin-4-amine
N-[(1R)-1-(3-bromophenyl)ethyI]-6-ethoxy-2-methylpyrido[3,2-cl]pyrimidin-4-amine
N-[(1R)-1-(3-bromophenyl)ethyI]-6-(2-methoxyethoxy)-2-methylpyrido[3,2-cl]pyrimidin-4-
N-[(1R)-1-(3-bromophenyl)ethyI]-6-(2-methoxyethoxy)-2-methylpyrido[3,2-cl]pyrimidin-4-
N-[(1R)-1-(3-bromophenyl)ethyI]-2-methyl-6-phenoxypyrido[3,2-cl]pyrimidin-4-amine
6-(benzyloxy)-N-[(1R)-1-(3-bromophenyl)ethyI]-2-methylpyrido[3,2-cl]pyrimidin-4-amine
N-[(1R)-1-(3-bromophenyl)ethyI]-6-fluoro-2-methylpyrido[3,4-cl]pyrimidin-4-amine
N-[1-(5-bromothiophen-2-yl)ethyI]-6-fluoro-2-methylpyrido[3,4-cl]pyrimidin-4-amine
6-fluoro-2-methyl-N-[1-(5-{2-[(methylamino)methyl]phenyl}thiophen-2-yl)ethyl]pyrido[3,4-d]pyrimidin-4-
amine
4-{[(2-methy1-4-{[1-(5-{2-[(methylamino)methyl]phenyl}thiophen-2-yl)ethyl]amino}pyrido[3,4-
d]pyrimidin-6-yl)oxy]methyllpiperidin-2-one
1-14-[(2-methy1-4-{[1-(5-{2-[(methylamino)methyl]phenyl}thiophen-2-yl)ethyl]amino}pyrido[3,4-
d]pyrimidin-6-yl)oxy]phenyllpyrrolidin-2-one
N4-[(1R)-1-(3-bromophenyl)ethyI]-2-methyl-N6-[2-(morpholin-4-yl)ethyl]pyrido[3,4-cl]pyrimidine-4,6-
diamine
3-[(4-{[(1R)-1-(3-bromophenyl)ethyl]amino}-2-methylpyrido[3,4-cl]pyrimidin-6-yl)oxy]phenol
N-[(1R)-1-(3-bromophenyl)ethy1]-2-methyl-6-[3-(1-methyl-4,5-dihydro-1H-imidazol-2-
yl)phenoxy]pyrido[3,4-cl]pyrimidin-4-amine
N-[1-(4-{[(1R)-1-(3-bromophenyl)ethyl]amino}-2-methylpyrido[3,4-cl]pyrimidin-6-yl)pyrrolidin-3-
yl]acetamide
N-[(1R)-1-(3-bromophenyl)ethy1]-2-methyl-644-(pyridin-3-ylmethyl)piperazin-1-yl]pyrido[3,4-
cl]pyrimidin-4-amine
N-12-[(4-{[(1R)-1-(3-bromophenyl)ethyl]amino}-2-methylpyrido[3,4-cl]pyrimidin-6-
yl)amino]ethyllacetamide
4-(4-{[(1R)-1-(3-bromophenyl)ethyl]amino}-2-methylpyrido[3,4-cl]pyrimidin-6-yl)piperazin-2-one
N4-[(1R)-1-(3-bromophenyl)ethyI]-2-methyl-N6-(tetrahydro-2H-pyran-4-yl)pyrido[3,4-d]pyrimidine-
4,6-diamine
N41-(4-{[1-(5-{2-[(dimethylamino)methyl]phenyl}thiophen-2-yl)ethyl]amino}-2-methylpyrido[3,4-
d]pyrimidin-6-y1)pyrrolidin-3-yl]acetamide
N441-(5-{2-[(dimethylamino)methyl]pheny11-2-thienyl)ethyl]-2-methyl-N642-(morpholin-4-
yl)ethyl]pyrido[3,4-d]pyrimidine-4,6-diamine
N4-[(1R)-1-(3-bromophenyl)ethyI]-2-methyl-N6-[2-(1H-pyrazol-1-yl)ethyl]pyrido[3,4-d]pyrimidine-4,6-
diamine
4-(4-{[1-(5-{2-[(dimethylamino)methyl]phenyl}thiophen-2-yl)ethyl]amino}-2-methylpyrido[3,4-d]pyrimidin-
6-y1)piperazin-2-one
N-12-[(4-{[1-(5-{2-[(dimethylamino)methyl]phenyl}thiophen-2-yl)ethyl]amino}-2-methylpyrido[3,4-
d]pyrimidin-6-yl)amino]ethyllacetamide
3-[(4-{[1-(5-{2-[(dimethylamino)methyl]phenyl}thiophen-2-yl)ethyl]amino}-2-methylpyrido[3,4-
d]pyrimidin-6-y1)oxy]phenol
N441-(5-{2-[(dimethylamino)methyl]pheny11-2-thienyl)ethyl]-2-methyl-N6-(tetrahydro-2H-pyran-4-
yl)pyrido[3,4-d]pyrimidine-4,6-diamine
N441-(5-{2-[(dimethylamino)methyl]pheny11-2-thienyl)ethyl]-2-methyl-N642-(1H-pyrazol-1-
yl)ethyl]pyrido[3,4-d]pyrimidine-4,6-diamine
N41-(5-{2-[(dimethylamino)methyl]phenyl}thiophen-2-yl)ethyl]-2-methyl-644-(pyridin-3-
ylmethyl)piperazin-1-yl]pyrido[3,4-d]pyrimidin-4-amine
N4-[1-(5-{2-[(dimethylamino)methyl]pheny11-2-thienyl)ethyl]-N6-[2-(1H-imidazol-1-yl)ethyl]-2-
methylpyrido[3,4-d]pyrimidine-4,6-diamine
N-[(1R)-1-(3-bromophenyl)ethyI]-6-methoxy-2-methylpyrido[3,4-d]pyrimidin-4-amine
N-[1-(5-{2-[(dimethylamino)methyl]phenyl}thiophen-2-yl)ethyl]-2-methyl-6-[3-(1-methyl-4,5-dihydro-1H-
imidazol-2-yl)phenoxy]pyrido[3,4-d]pyrimidin-4-amine
N-[1-(5-{2-[(dimethylamino)methyl]phenyl}thiophen-2-yl)ethyl]-2-methyl-6-(methylsulfanyl)pyrido[3,4-
d]pyrimidin-4-amine
N-[(3R)-1-(4-{[1-(5-{2-[(dimethylamino)methyl]phenyl}thiophen-2-yl)ethyl]amino}-2-methylpyrido[3,4-
d]pyrimidin-6-y1)pyrrolidin-3-yl]acetamide
N-[(35)-1-(4-{[(15)-1-(5-{2-[(dimethylamino)methyl]phenyl}thiophen-2-yl)ethyl]amino}-2-methylpyrido[3,4-
d]pyrimidin-6-yl)pyrrolidin-3-yl]acetamide
N-12-[(4-{[(1R)-1-(3-bromophenyl)ethyl]amino}-2-methylpyrido[3,4-d]pyrimidin-6-yl)oxy]ethyllacetamide
N-[1-(5-{2-[(dimethylamino)methyl]phenyl}thiophen-2-yl)ethyl]-6-ethoxy-2-methylpyrido[3,4-
d]pyrimidin-4-amine
N-[1-(5-{2-[(dimethylamino)methyl]phenyl}thiophen-2-yl)ethyl]-6-methoxy-2-methylpyrido[3,4-
d]pyrimidin-4-amine
N-[1-(5-{2-[(dimethylamino)methyl]phenyl}thiophen-2-yl)ethyl]-2-methyl-6-propoxypyrido[3,4-
d]pyrimidin-4-amine
6-[2-(dimethylamino)ethoxy]-N-[1-(5-{2-[(dimethylamino)methyl]-phenyl}thiophen-2-yl)ethyl]-2-
methylpyrido[3,4-d]pyrimidin-4-amine
N-[1-(5-{2-[(dimethylamino)methyl]phenyl}thiophen-2-yl)ethyl]-6-[(1,1-dioxidotetrahydro-2H-thiopyran-4-
yl)oxy]-2-methylpyrido[3,4-d]pyrimidin-4-amine
[2-(5-{1-[(6-fluoro-2-methylpyrido[3,4-d]pyrimidin-4-yl)amino]ethyllthiophen-2-
yl)phenyl]methanol
3-(4-{[1-(5-{2-[(dimethylamino)methyl]phenyl}thiophen-2-yl)ethyl]amino}-2-methylpyrido[3,4-
d]pyrimidin-6-yl)-1-methylpyrrolidin-2-one
N-[(1R)-1-(3-bromophenyl)ethyI]-6-[3-(dimethylamino)propoxy]-2-methylpyrido[3,4-d]pyrimidin-4-amine
6-(azetidin-1-yl)-N41-(5-{2-
[(dimethylamino)methyl]phenyl}thiophen-2-yl)ethyl]-2-methylpyrido[3,4-d]pyrimidin-4-amine
N41-(5-{2-[(dimethylamino)methyl]phenyl}thiophen-2-yl)ethyl]-643-(dimethylamino)propoxy]-2-
methylpyrido[3,4-d]pyrimidin-4-amine
6-(cyclopropylmethoxy)-N41-(5-{2-[(dimethylamino)methyl]phenyll-thiophen-2-yl)ethyl]-2-
methylpyrido[3,4-d]pyrimidin-4-amine
2-[(4-{[1-(5-{2-[(dimethylamino)methyl]phenyl}thiophen-2-yl)ethyl]amino}-2-methylpyrido[3,4-
d]pyrimidin-6-yl)oxy]ethanol
N-[(1R)-1-(3-bromophenyl)ethyI]-6-(cyclopropylmethoxy)-2-methylpyrido[3,4-d]pyrimidin-4-amine
3-[(4-{[1-(5-{2-[(dimethylamino)methyl]phenyl}thiophen-2-yl)ethyl]amino}-2-methylpyrido[3,4-d]pyrimidin-
6-yl)oxy]propan-1-ol
N-[1-(5-{2-[(dimethylamino)methyl]phenyl1-2-thienyl)ethyl]-6-[(1-imino-1-oxidohexahydro-11ambda4-
thiopyran-4-yl)oxy]-2-methylpyrido[3,4-d]pyrimidin-4-amine
N-[(1R)-1-(3-bromophenyl)ethyI]-6-[2-(dimethylamino)ethoxy]-2-methylpyrido[3,4-d]pyrimidin-4-amine
N-[(3R)-1-(4-{[1-(5-{2-[(dimethylamino)methyl]phenyl}thiophen-2-yl)ethyl]amino}-2-methylpyrido[3,4-
d]pyrimidin-6-yl)pyrrolidin-3-yl]acetamide (diastereomer 1)
N-[(3R)-1-(4-{[1-(5-{2-[(dimethylamino)methyl]phenyl}thiophen-2-yl)ethyl]amino}-2-methylpyrido[3,4-
d]pyrimidin-6-yl)pyrrolidin-3-yl]acetamide (diastereomer 2)
N-[(3S)-1-(4-{[1-(5-{2-[(dimethylamino)methyl]phenyl}thiophen-2-yl)ethyl]amino}-2-methylpyrido[3,4-
d]pyrimidin-6-yl)pyrrolidin-3-yl]acetamide (diastereomer 1)
N-[(3S)-1-(4-{[1-(5-{2-[(dimethylamino)methyl]phenyl}thiophen-2-yl)ethyl]amino}-2-methylpyrido[3,4-
d]pyrimidin-6-yl)pyrrolidin-3-yl]acetamide (diastereomer 2)
N4-[(1R)-1-(3-bromophenyl)ethyI]-2-methyl-N6-[2-(methylsulfonyl)ethyl]pyrido[3,4-
d]pyrimidine-4,6-diamine
N4-[(1R)-1-(3-bromophenyl)ethyl]-N6-[2-(1H-imidazol-1-yl)ethyl]-2-methylpyrido[3,4-d]pyrimidine-4,6-
diamine
3-(4-{[1-(5-{2-[(dimethylamino)methyl]phenyl}thiophen-2-yl)ethyl]-amino}-2-methylpyrido[3,4-
d]pyrimidin-6-yl)-3-hydroxy-1-methylpyrrolidin-2-one
4-{[(4-{[1-(5-{2-[(dimethylamino)methyl]phenyl}thiophen-2-yl)ethyl]amino}-2-methylpyrido[3,4-
d]pyrimidin-6-yl)amino]methyllpiperidin-2-one
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]-4-[4,5-dimethyl-2-(pyridin-2-yl)-1H-imidazol-1-yl]butanamide
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]benzamide
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]-2-phenylacetamide
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]cyclohexanecarboxamide
2-cyclopropyl-N-[1-(4-{[1-(5-{2-[(dimethylamino)methyl]phenyl}thiophen-2-yl)ethyl]amino}-2-
methylpyrido[3,4-d]pyrimidin-6-yl)azetidin-3-yl]acetamide
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]-2-(morpholin-4-yl)acetamide
6-(3-aminoazetidin-1-yl)-N-[1-(5-{2-[(dimethylamino)methyl]phenyl}thiophen-2-yl)ethyl]-2-methylpyrido[3,4-
d]pyrimidin-4-amine
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]benzenesulfonamide
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]-1-phenylmethanesulfonamide
1-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]-3-phenylurea
tert-butyl (3R)-3-[acetyl(3-{[1-(4-{[1-(5-{2-[(dimethylamino)methyl]phenyl}-2-thienyl)ethyl]amino}-2-
methylpyrido[3,4-d]pyrimidin-6-yl)azetidin-3-yl]carbamoyl}-phenyl)amino]pyrrolidine-1-carboxylate
tert-butyl 4-[acetyl(3-{[1-(4-{[1-(5-{2-[(dimethylamino)methyl]phenyl}thiophen-2-yl)ethyl]amino}-2-
methylpyrido[3,4-d]pyrimidin-6-yl)azetidin-3-yl]carbamoyl}-phenyl)amino]piperidine-1-carboxylate
6-[3-(benzylamino)azetidin-1-yl]-N-[1-(5-{2-[(dimethylamino)methyl]-phenyl}thiophen-2-yl)ethyl]-2-
methylpyrido[3,4-d]pyrimidin-4-amine
N-[1-(4-{[1-(5-{2-[(dimethylamino)methyl]phenyl}thiophen-2-yl)ethyl]amino}-2-methylpyrido[3,4-
d]pyrimidin-6-yl)azetidin-3-yl]thiomorpholine-4-carboxamide 1,1-dioxide

In some embodiments, the SOS inhibitor is

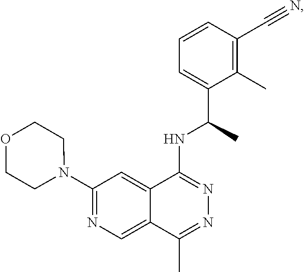 [SMILES: Cc1c(C#N)cccc1[C@@H](C)Nc1nnc(C)c2cnc(N3CCOCC3)cc12]

or a stereoisomer, solvate, or salt thereof.

In embodiments of Formula A to F, N to Z, Z′, AA, AA′, BB, BC, and BC′, the term “Aryl” refers to a radical derived from an aromatic monocyclic or multicyclic hydrocarbon ring system by removing a hydrogen atom from a ring carbon atom. In embodiments of Formula A to F, N to Z, Z′, AA, AA′, BB, BC, and BC′, the aromatic monocyclic or multicyclic hydrocarbon ring system contains only hydrogen and carbon from five to eighteen carbon atoms, where at least one of the rings in the ring system is frilly unsaturated, i.e., it contains a cyclic, delocalized (4n+2) π-electron system in accordance with the Hückel theory. In embodiments of Formula A to F, N to Z, Z′, AA, AA′, BB, BC, and BC′, unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

In embodiments of Formula A to F, N to Z, Z′, AA, AA′, BB, BC, and BC′, the term “Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In embodiments of Formula A to F, N to Z, Z′, AA, AA′, BB, BC, and BC′, the carbocyclyl is attached to the rest of the molecule by a single bond. In embodiments of Formula A to F, N to Z, Z′, AA, AA′, BB, BC, and BC′, the term carbocyclyl is saturated (i.e., containing single C—C bonds only) or unsaturated (i.e., containing one or more double bonds or triple bonds). In embodiments of Formula A to F, N to Z, Z′, AA, AA′, BB, BC, and BC′, fully saturated carbocyclyl radical is also referred to as “cycloalkyl. In embodiments of Formula A to F, N to Z, Z′, AA, AA′, BB, BC, and BC′, an unsaturated carbocyclyl is also referred to as “cycloalkenyl.” In embodiments of Formula A to F, N to Z, Z′, AA, AA′, BB, BC, and BC′, unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

In embodiments of Formula A to F, N to Z, Z′, AA, AA′, BB, BC, and BC′, the term “Heterocyclyl” refers to a stable 3- to 18-membered non-aromatic ring radical that comprises two to twelve carbon atoms and from one to six heteroatoms selected from nitrogen, oxygen and sulfur. In embodiments of Formula A to F, N to Z, Z′, AA, AA′, BB, BC, and BC′, unless stated otherwise specifically in the specification, the heterocyclyl radical is a monocyclic, bicyclic, tricyclic or tetracyclic ring system, which optionally includes fused or bridged ring systems. In embodiments of Formula A to F, N to Z, Z′, AA, AA′, BB, BC, and BC′, the heteroatoms in the heterocyclyl radical are optionally oxidized. In embodiments of Formula A to F, N to Z, Z′, AA, AA′, BB, BC, and BC′, one or more nitrogen atoms, if present, are optionally quaternized. In embodiments of Formula A to F, N to Z, Z′, AA, AA′, BB, BC, and BC′, the heterocyclyl radical is partially or fully saturated. In embodiments of Formula A to F, N to Z, Z′, AA, AA′, BB, BC, and BC′, the fully saturated carbocyclyl radical is also referred to as “heterocycloalkyl.” In embodiments of Formula A to F, N to Z, Z′, AA, AA′, BB, BC, and BC′, the heterocyclyl is attached to the rest of the molecule through any atom of the ring(s). In embodiments of Formula A to F, N to Z, Z′, AA, AA′, BB, BC, and BC′, unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

In embodiments of Formula A to F, N to Z, Z′, AA, AA′, BB, BC, and BC′, the term “Heteroaryl” refers to a radical derived from a 3- to 18-membered aromatic ring radical that comprises two to seventeen carbon atoms and from one to six heteroatoms selected from nitrogen, oxygen and sulfur. In embodiments of Formula A to F, N to Z, Z′, AA, AA′, BB, BC, and BC′, as used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) n-electron system in accordance with the Hückel theory. In embodiments of Formula A to F, N to Z, Z′, AA, AA′, BB, BC, and BC′, the heteroaryl includes fused or bridged ring systems. In embodiments of Formula A to F, N to Z, Z′, AA, AA′, BB, BC, and BC′, the heteroatom(s) in the heteroaryl radical is optionally oxidized. In embodiments of Formula A to F, N to Z, Z′, AA, AA′, BB, BC, and BC′, the one or more nitrogen atoms, if present, are optionally quaternized. In embodiments of Formula A to F, N to Z, Z′, AA, AA′, BB, BC, and BC′, the heteroaryl is attached to the rest of the molecule through any atom of the ring(s). In embodiments of Formula A to F, N to Z, Z′, AA, AA′, BB, BC, and BC′, unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

In embodiments, the SOS1 inhibitor has the formula (CF) immediately below, or a pharmaceutically acceptable salt or solvate thereof:

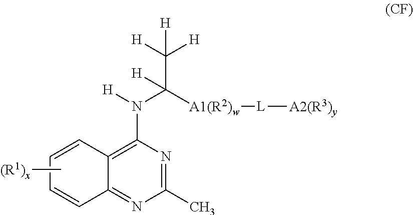

in which
R¹ stands for
   a substituent independently selected from: a hydrogen atom, a halogen atom, a hydroxy, cyano, nitro, C₁-C₆-alkylsulfanyl or an amino group-NR^{a}R^{b},
      wherein R^{a} and R^{b} are selected independently from a hydrogen atom or a C₁-C₆-alkyl,
   a substituent selected from: a C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl,
   —C(═O)OH, —C(═O)OR^{c}, and wherein R^{c} stands for C₁-C₆-alkyl, C₃-C₆-alkenyl, C₃-C₆-alkynyl, C₃-C₈-cycloalkyl or C₄-C₈-cycloalkenyl,
   —N═S(═O)(R^{d})R^{e}, and wherein R^{d} and R^{e} are selected independently from hydrogen, C₁-C₆-alkyl, C₂-C₆-alkenyl, C₂-C₆-alkynyl, C₃-C₈-cycloalkyl or C₄-C₈-cycloalkenyl,
   —NH—C(O)— C₁-C₆-alkyl, —NH—C(O)—NR^{a}R^{b}, wherein R^{a} and R^{b} are selected independently from a hydrogen atom or a C₁-C₆-alkyl, —NH—(CH₂)k-NH—C(O)— C₁-C₆-alkyl, wherein k is 1 or 2, —NH—(CH₂)ₗ—R^{f}, wherein
      l is 0, 1 or 2 and R^{f}stands for a 4- to 7-membered heterocycloalkyl, heteroaryl, C₁-C₆-alkylsulfonyl,
      whereby in all foregoing definitions the C₁-C₆-alkyl-, C₁-C₆-alkoxy-, the 4- to 7-membered heterocycloalkyl and the heteroaryl can be optionally substituted, one, two or three times, identically or differently, with:
         a halogen atom, or a group selected from hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy-, C₁-C₆-alkylsulfonyl, phenyl, benzyl-, heteroaryl, —(CH₂)-heteroaryl-, C₃-C₈-cycloalkoxy-, phenyloxy-, heteroaryloxy-, —NH—C(O)— C₁-C₆-alkyl or an amino group —NR^{a}R^{b}, wherein R^{a} and R^{b} are selected independently from a hydrogen atom or a C₁-C₆-alkyl, or a substituent

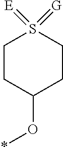

wherein E and G each stands for an electron pair, or one of E and G stands for an electron pair and the other for an oxygen atom or a group ═NH or ═N— C₁-C₄-alkyl, or one of E and G stands for an oxygen atom and the other one for a group ═NH or ═N— C₁-C₄-alkyl, or E and G each stands for an oxygen atom or each stands for a group ═NH or ═N— C₁-C₄-alkyl, or
a substituent —O—(CH₂)_{z}-phenyl, —O—(CH₂)_{z}—C₄-C₇-heterocycloalkyl, —O—(CH₂)z-heteroaryl, whereby
   z is 0, 1 or 2, and the phenyl, heterocycloalkyl and heteroaryl can optionally be substituted with a group selected from hydroxy, heterocycloalkyl or heterocycloalkenyl, which both can be substituted with a methyl- and/or oxo group,
or a substituent selected from the group of

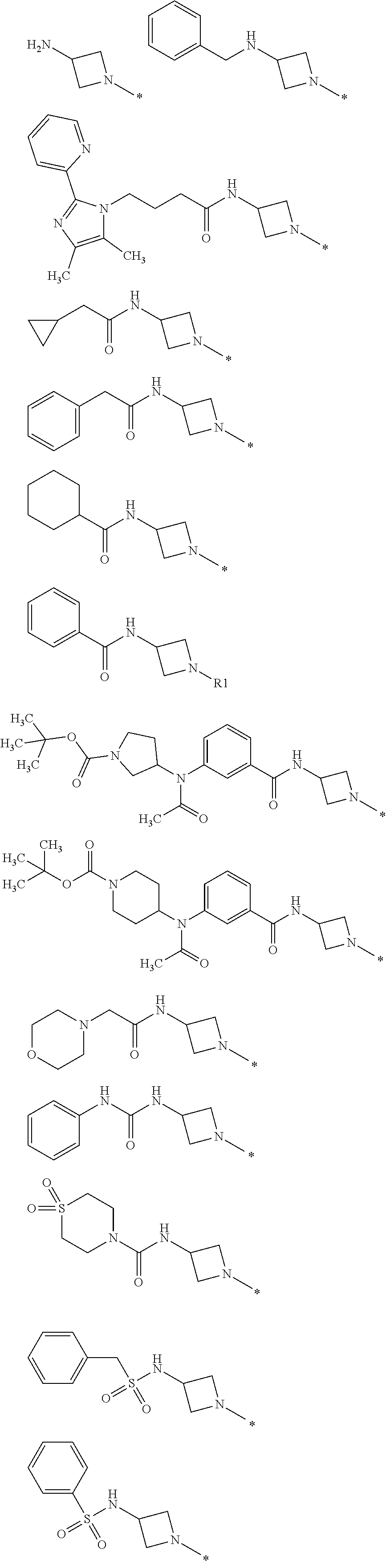

and wherein x is 1, 2 or 3,
A1 stands for
   a C₄ to C₁₂ carbocyclic, heterocyclic, optionally bicyclic, optionally aromatic or optionally heteroaromatic ring system, wherein in a bicyclic aromatic or
   heteroaromatic ring system one or two double bonds can be hydrogenated,
R² stands for
   a hydrogen atom, a hydroxy group, oxo (═O), a halogen atom, a cyano group, a substituent selected from: a C₁-C₆-alkyl, C₁-C₆-alkoxy-, C₂-C₆-alkenyl, C₂-C₆-alkynyl,
   C₃-C₈-cycloalkyl, C₄-C₈-cycloalkenyl, 4- to 7-membered heterocycloalkyl, —O—CH₂-4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl, C₁-C₆-alkylsulfonyl,
   —NR^{a}R^{b}, wherein R^{a} and R^{b} are selected independently from a hydrogen atom or a C₁-C₆-alkyl,
   —C(O)—NR^{a}R^{b}, wherein R^{a} and R^{b} are selected independently from a hydrogen atom or a C₁-C₆-alkyl, —C(O)—O—R⁹, wherein R⁹ is a hydrogen atom or a C₁-C₆-alkyl, —O—R¹, wherein Rh is a C₁-C₆-alkyl or —CH₂—NR^{a}R^{b}, wherein R^{a} and R^{b} are selected independently from a hydrogen atom or a C₁-C₆-alkyl,
and w is 1 or 2,
and wherein
A2(R³)y stands either for a hydrogen atom or
A2 has the same meanings as the substituent A1 and
R3 stands for
   a hydrogen atom, a halogen atom, a hydroxy, oxo, cyano, nitro group, a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, C₁-C₆-haloalkyl,
      which substituent is optionally substituted, one, two or three times, identically or differently, with a substituent selected from:
         a halogen atom, or a group selected from hydroxy, oxo (═O), cyano, C₁-C₆-alkyl, C₁-C₆-alkoxy, C₁-C₆-haloalkyl, phenyl, —C(O)NRⁱR^{j}, wherein
         Rⁱ and R^{j} are selected independently from a hydrogen atom or a C₁-C₆-alkyl, heteroaryl,
      or with amino —NR^{k}R^{l}, wherein R^{k} and R^{l} are selected independently from
         a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
         one, two or three times, identically or differently, with a substituent selected from C₁-C₆-haloalkyl, hydroxyl, oxo (═O), phenyl, cyano, C₁-C₆-alkoxy, heteroaryl, wherein
         the heteroaryl can optionally be substituted with a methyl group, or —CH₂—C(O)—R^{m}, wherein
         R^{m} is a bicyclic heteroaryl, which can be partially hydrogenated, a C₁-C₆-alkoxy or a group —NRⁿR^{o}, in which
         Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
         -NRⁿR^{o} stands for a 4- to 7-membered azacycloalkyl, bound via the nitrogen atom to the rest of the molecule and which optionally contains one more heteroatom selected from nitrogen and oxygen;
         —C(═O)R^{p}, wherein R^{p} is selected from
         the group of a C₁-C₆-alkoxy, a C₁-C₆-alkyl, which is optionally substituted, one, two or three times, identically or differently, with a substituent selected from hydroxyl or C₁-C₆-alkoxy, a mono- or bicyclic heteroaryl, a 4- to 7-membered heterocycloalkyl or R^{p} is a group —CH₂—NR^{q}R^{r}; wherein R^{q} and R^{f} are selected independently from hydrogen, phenyl or a C₁-C₆-alkyl, which may optionally be substituted up to threefold with fluorine, —NR^{s}R^{t} is
         a 4- to 7-membered azacycloalkyl, bound via the nitrogen atom to the rest of the molecule, or a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein R^{u} is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
   —C(═O)R^{v}, —C(═O)NH₂, —C(═O)N(H)R^{v}, —C(═O)N(R^{v})R^{w}, —C(═O)OR^{v}, wherein
      R^{v} and R^{w} represent, independently from each other, a group selected from hydrogen, C₁-C₄-alkyl, C₁-C₄-haloalkyl, phenyl, or a group —(CH₂)₂—NR^{x}R^{y}, wherein R^{x} and R^{y} independently from each other stand for hydrogen, a C₁-C₄-alkyl or a group —(CH₂)₂N(CH₃)₂;
   —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein R^{z} and R^{za} represent, independently from each other, a group selected from C₁-C₄-alkyl, C₁-C₄-haloalkyl and phenyl,
   C₁-C₆-alkoxy-, C₁-C₆-haloalkoxy-, —O—(CH₂)₈— C₃-C₈-cycloalkyl, —O—(CH₂)₈-phenyl, —O—(CH₂)₈-heterocycloalkyl, —O—(CH₂)₈-heteroaryl, s is 0, 1, 2 or 3,
   —S(═O)₂R^{z}, —S(═O)₂NH₂, —S(═O)₂NHR^{z}, —S(═O)₂N(R^{z})R^{za}, wherein R^{z} and R^{za} represent, independently from each other, a group selected from C₁-C₄-alkyl, C₁-C₄-haloalkyl and phenyl,
wherein y is 1, 2 or 3, and
L stands either for a bond or for —O—(CH₂)k, wherein k is 0, 1, 2 or 3, or a group —CH═CH—(CH₂)n, wherein n is 0, 1 or 2,
or a tautomer, an N-oxide, a hydrate, a solvate, or a salt thereof, or a mixture of same;
wherein, as used in this embodiment, the term “fused heterocycloalkyl” means a bicyclic, saturated heterocycle with 6, 7, 8, 9 or 10 ring atoms in total, in which the two rings share two adjacent ring atoms, which“fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom; the term “bridged heterocycloalkyl”, as used in this embodiment, means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom; the term “cycloalkenyl”, as used in this embodiment, means a monovalent, mono- or bicyclic hydrocarbon ring which contains at least one double bond; the term “spirocycloalkyl”, as used in this embodiment, means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom; the term “azacycloalkyl”, as used in this embodiment, means a monocyclic saturated heterocycyl which is attached to the rest of the molecule via the nitrogen atom and which optionally contains one more heteroatom selected from nitrogen and oxygen; the term “heterocycloalkenyl”, as used in this embodiment, means a monocyclic, unsaturated, non aromatic heterocycle, which contains at least one double bond and one or two identical or different ring heteroatoms from the series: N, O, S; it being possible for said heterocycloalkenyl group to be attached to the rest of the molecule via any one of the carbon atoms or, if present, a nitrogen atom; the term “heterospirocycloalkyl”, as used in this embodiment, means a bicyclic, maturated heterocycle with 6, 7, 8, 9, 10 or 11 ring atoms in total, in which the two rings share one common ring carbon atom, which “heterospirocycloalkyl”, as used in this embodiment, contains one, two or three identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

In embodiments, the SOS1 inhibitor has the formula (CG) immediately below, or a pharmaceutically acceptable salt or solvate thereof:

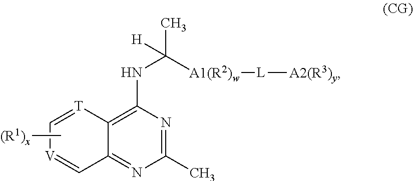

in which
R¹ stands for
   a substituent independently selected from: a hydrogen atom, a halogen atom, a hydroxy, cyano, nitro, C₄-C₈-alkylsulfanyl or an amino group —NR^{a}R^{b},
   wherein R^{a} and R^{b} are selected independently from a hydrogen atom or a C₁-C₆-alkyl, a substituent selected from: a C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to I0-membered heterocycloalkenyl, heterospirocycloalkyl, fused heterocycloalkyl, bridged heterocycloalkyl, phenyl, heteroaryl, C₃-C₆-haloalkyl,
   —C(═O)OH, —C(═O)OR^{c}, and wherein R^{c} stands for C₁-C₆-alkyl, C₃-C₆-alkenyl, C₃-C₆-alkynyl, C₃-C₈-cycloalkyl or C₄-C₈-cycloalkenyl,
   —N═S(═O)(R^{d})R^{e}, and wherein R^{d} and R^{e} are selected independently from hydrogen, C₃-C₆-alkyl, C₂-C₆-alkenyl, C₂-C₆-alkynyl, C₃-C₈-cycloalkyl or C₄-C₈-cycloalkenyl,
   —NH—C(O)—C₁-C₆-alkyl, —NH—C(O)—NR^{a}R^{b}, wherein R^{a} and R^{b} are selected independently from a hydrogen atom or a C₁-C₆-alkyl, —NH—(C₁H₂)ₖ—NH—C(O)—C₁-C₆-alkyl, wherein k is 1 or 2, —NH—(CH)ₗ—R^{f}, wherein
      l is 0, 1 or 2 and R^{f} stands for a 4- to 7-membered heterocycloalkyl, heteroaryl, C₁-C₆-alkylsulfonyl,
      whereby in all foregoing definitions of this embodiment the C₁-C₆-alkyl, C₁-C₆-alkoxy, the 4- to 7-membered heterocycloalkyl and the heteroaryl can be optionally substituted, one, two or three times, identically or differently, with:
         a halogen atom, or a group selected from hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy-, C₁-C₆-alkylsulfonyl, phenyl, benzyl-, heteroaryl, —(CH₂)-heteroaryl, C₃-C₈-cycloalkoxy, phenyloxy-, heteroaryloxy-, —NH₁—C(O)—C₁-C₆-alkyl or an amino group —NR^{a}R^{b}, wherein R^{a} and R^{b} are selected independently from a hydrogen atom or a C₁-C₆-alkyl, or
   a substituent

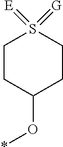

wherein E and G each stands for an electron pair, or one of E and G stands for an electron pair and the other for an oxygen atom or a group ═NH or ═N—C₁-C₄-alkyl, or one of E and G stands for an oxygen atom and the other one for a group ═NH or ═N—C₁-C₄-alkyl, or E and G each stands for an oxygen atom or each stands for a group ═NH or ═N—C₁-C₄-alkyl, or
a substituent —O—(CH₂)_{z}-phenyl, —O—(CH₂)_{z}—C₄-C₇-heterocycloalkyl, —O—(CH₂)_{z}-heteroaryl, whereby
   z is 0, 1 or 2, and the phenyl, heterocycloalkyl and heteroaryl can optionally be substituted with a group selected from hydroxy, heterocycloalkyl or heterocycloalkenyl, which both can be substituted with a methyl- and/or oxo-group,
and wherein x is 1, 2 or 3,
A1 stands for
   a C₄ to C₁₂ carbocyclic, heterocyclic, optionally bicyclic, optionally aromatic or optionally heteroaromatic ring system, wherein in a bicyclic aromatic or heteroaromatic ring system one or two double bonds can be hydrogenated,
R² stands for
   a hydrogen atom, a hydroxy group, oxo (═O), a halogen atom, a cyano group, a substituent selected from: a C₁-C₆-alkyl, C₁-C₆-alkoxy-, C₂-C₆-alkenyl, C₂-C₅-alkynyl, C₃—C&-cycloalkyl, C₄-C₈-cycloalkenyl, 4- to 7-membered heterocycloalkyl, —O—CH₂-4- to 7-membered heterocycloalkyl, 5- to I0-membered heterocycloalkenyl, heterospirocycloalkyl, fused heterocycloalkyl, bridged heterocycloalkyl, phenyl, heteroaryl, C₃-C₈-haloalkyl, C₁-C₆-alkylsulfonyl,
   —NR^{a}R^{b}, wherein R^{a} and R^{b} are selected independently from a hydrogen atom or a C₃-C₈-alkyl,
   —C(O)—NR^{a}R^{b}, wherein R^{a} and Rh are selected independently from a hydrogen atom or a C₁-C₆-alkyl,
   —C(O)—O—R^{g}, wherein R^{g} is a hydrogen atom or a C₁-C₆-alkyl,
—O—R^{h}, wherein R^{h} is a C₁-C₆-alkyl or —CH₂—NR^{a}R^{b}, wherein R^{a} and R^{b} are selected independently from a hydrogen atom or a C₁-C₆-alkyl,
and w is 1 or 2,
and wherein
A2(R³)y stands either for a hydrogen atom or
A2 has the same meanings as the substituent A1 and
R³ stands for
   a hydrogen atom, a halogen atom, a hydroxy, oxo, cyano, nitro group, a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, C₁-C₆-haloalkyl,
      which substituent is optionally substituted, one, two or three times, identically or differently, with a substituent selected from:
         a halogen atom, or a group selected from hydroxy, oxo (═O), cyano, C₁-C₆-alkyl, C₁-C₆-alkoxy, C₁-C₆-haloalkyl, phenyl, —C(O)NRⁱR^{j}, wherein
         Rⁱ and R^{j} are selected independently from a hydrogen atom or a C₁-C₆-alkyl, heteroaryl,
         or with amino —NR^{k}R^{l}, wherein R^{k} and R^{l} are selected independently from
         a hydrogen atom, a substituent selected from a C₁-C₆-alkyl, C₂-C₆-alkenyl, C₂-C₆-alkynyl, C₃-C₈-cycloalkyl, C₃-C₆-alkylsulfonyl, phenyl, heteroaryl, 4- to 7-membered heterocycloalkyl, which are optionally substituted one, two or three times, identically or differently, with a substituent selected from C₁-C₆-haloalkyl, hydroxyl, oxo (═O), phenyl, cyano, C₁-C₆-alkoxy, heteroaryl, wherein
         the heteroaryl can optionally be substituted with a methyl group, or —CH₂—C(O)—R^{m}, wherein
         R^{m} is a bicyclic heteroaryl, which can be partially hydrogenated, a C₁-C₆-alkoxy or a group —NRⁿR^{o}, in which
         Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
         -NRⁿR^{o} stands for a 4- to 7-membered azacycloalkyl, bound via the nitrogen atom to the rest of the molecule and which optionally contains one more heteroatom selected from nitrogen and oxygen;
      —C(═O)R^{p}, wherein R^{p} is selected from
         the group of a C₁-C₆-alkoxy, a C₁-C₆-alkyl, which is optionally substituted, one, two or three times, identically or differently, with a substituent selected from hydroxyl or C₁-C₆-alkoxy, a mono- or bicyclic heteroaryl, a 4- to 7-membered heterocycloalkyl or R^{p} is a group —CH₂—NR^{q}R^{r}; wherein R^{q} and R^{r} are selected independently from hydrogen, phenyl or a C₁-C₆-alkyl, which may optionally be substituted up to threefold with fluorine,
      —NR^{s}R^{t} is
         a 4- to 7-membered azacycloalkyl, bound via the nitrogen atom to the rest of the molecule, or a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein R^{u} is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl, —C(═O)R^{v}, —C(═O)NH₂, —C(═O)N(H)R^{v}, —C(═O)N(R^{v})R^{w}, —C(═O)OR^{v}, wherein
         R^{v} and R^{w} represent, independently from each other, a group selected from hydrogen, C₁-C₄-alkyl, C₁-C₄-haloalkyl, phenyl, or a group —(CH₂)₂—NR^{x}R^{y}, wherein R^{x} and R^{y} independently from each other stand for hydrogen, a C₁-C₄-alkyl or a group —(CH₂)₂N(CH₃)₂;
   —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein
      R^{z} and R^{za} represent, independently from each other, a group selected from C₁-C₄-alkyl, C₁-C₄-haloalkyl and phenyl,
   C₁-C₆-alkoxy-, C₁-C₆-haloalkoxy-, —O—(CH₂), —C₃-C₈-cycloalkyl, —O—(CH₂)ₛ-phenyl, —O—(CH₂)ₛ-heterocycloalkyl, —O—(CH₂)r-heteroaryl, s is 0, 1, 2 or 3,
   —S(═O)₂R^{z}, —S(═O)₂NH₂, —S(═O)₂NHR^{z}, —S(═O)₂N(R^{z})R^{za}, wherein R^{z} and R^{za} represent, independently from each other, a group selected from C₁-C₄-alkyl, C₁-C₄-haloalkyl and phenyl,
wherein y is 1, 2 or 3, and
L stands either for a bond or for —O—(CH₂)ₖ, wherein k is 0, 1, 2 or 3, or a group —CH═CH—(CH₂)ₙ, wherein n is 0, 1 or 2,
and either both T and V stand for nitrogen or T stands for carbon and V for nitrogen or T for nitrogen and V for carbon,
or a tautomer, an N-oxide, a hydrate, a solvate, or a salt thereof, or a mixture of same;
wherein in embodiments, R¹ is selected from the list of the following substituents, H, *—OCH₃, *—OC₂H₅, *—CH₂OH, *—C(O)OH, *—C(O)OCH₃, —Br, *—O—

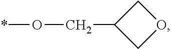 [SMILES: *OCC1COC1]

CH(CH₃)₂, *—O—(CH₂)₂CH(CH₃), *—O—(CH₂)₃CH₃, *—O—(CH₂)₂O—CH₃,

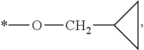 [SMILES: *OCC1CC1]

*—OCH₂—, Phenyl, *—N═S (O)(CH₃)₂, *—CH₃, *—NH(CH₃), *—N(CH₃)₂, *—NH₂,

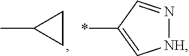

*—C(CH₃)₂—

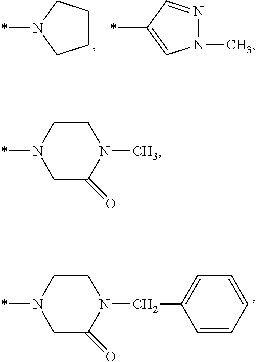

### OH,

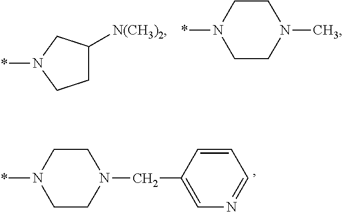 [SMILES: *N1CCC(N(C)C)C1 ; *N1CCN(C)CC1 ; *N1CCN(Cc2cccnc2)CC1]

*—NH—(CH₂)2-NH—C(O)—CH₃, *—NH—(CH₂)₂-morpholino,*—NH—C(O)—CH₃, —NH—C(O)—NH—CH, *—NH—C(O)—N(CH₃)₂, *—NO₂, —NH—S(O)₂—CH₃, *—N—S(O)(CH₃)₂, —OH—O—(CH₂)₂—S(O)₂—CH₃, fluorine,

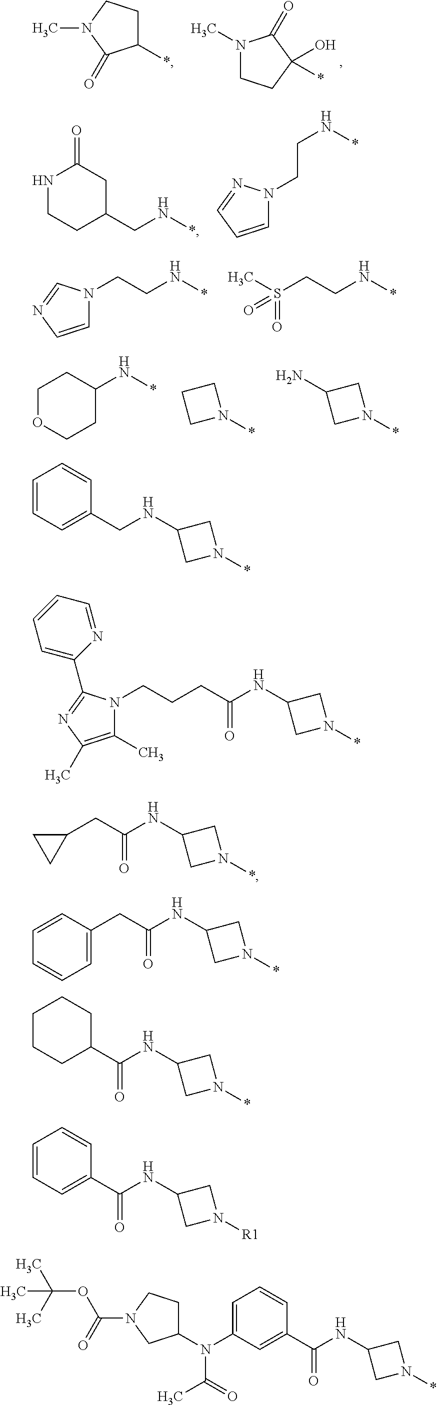

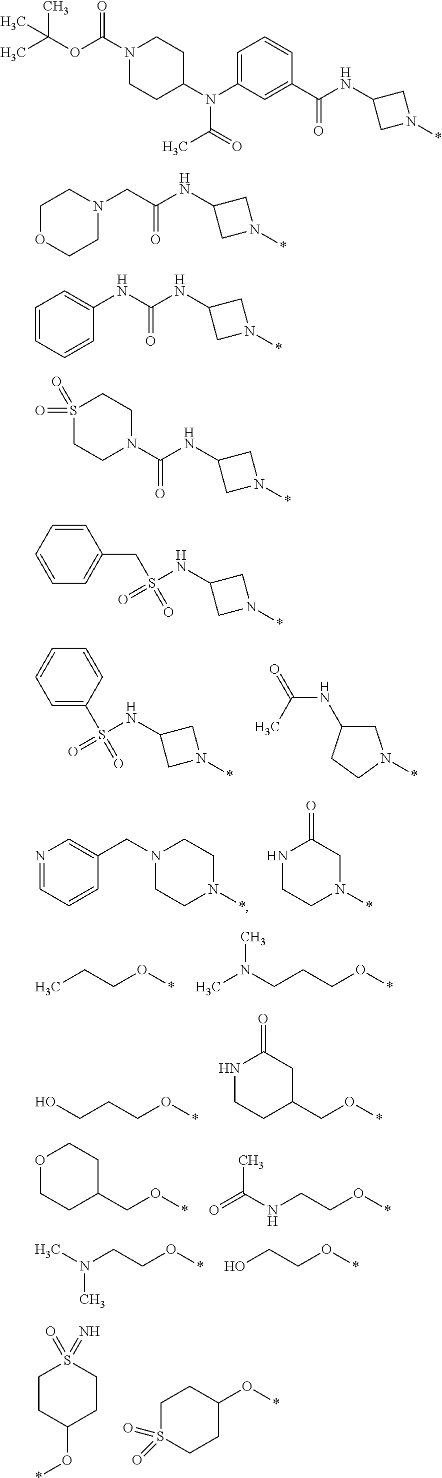

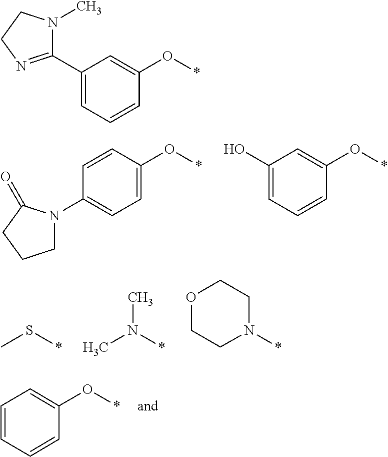

z is 1 or 2 and, x is 1 or 2; wherein “*” represents the point of attachment to the remainder of the compound;
wherein in embodiments, A1 is selected from the group,

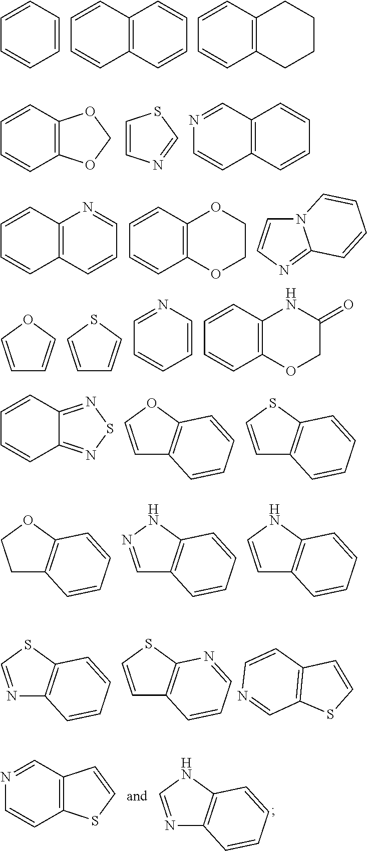

wherein in embodiments, R² is selected from the group of hydrogen, hydroxy, oxo (═O), cyano, cyclopropyl, 1,1-dimethylcyclopropyl, —C(═CH₂)CH₃, —C(CH₃)═CHCH₃, —CH═CH—(CH₂)₂CH₃, CH═CHCH₃, —CH═CH cyclopropyl), —C(O)NH₂, C(O)OCH₃, —S(O)₂CH₃, —OCH₃, —CH₂NH₂, a halogen atom (F, Cl; Br), and, w is 1 or 2;
wherein in embodiments, A2 is selected from the group,

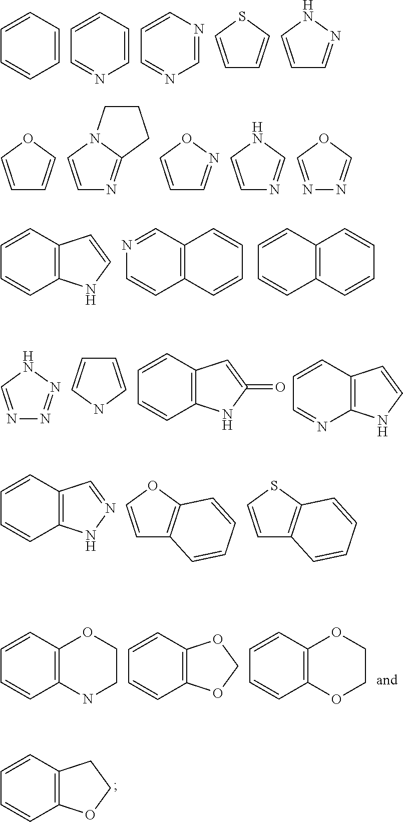

wherein in embodiments, R³ is selected from the group of the following substituents, *—C(O)NH₁—(CH₂)₂CH₃, *—C(O)—N(CH₃)₂, *—C(O)—NH₂, *—C(O)—NH—(CH₂)₂N(CH₃)₂, *—CH₂—C(O)—NH₂, hydrogen, *—F, *—Cl, *—Br, *—C═N; *—CF₃, *—CH₃, *—C₂H₅, *—CH═CH₂; *—CH₂—CN; *—CH(CH₃)—NH₂; *—CH═CH—CN; —C(O)—OH; *—C(O)—OCH₃; *—C(O)—CH₃; *—C(CH₃)₂—C(O)—OCH₃; *—C(CH₃)₂—CN; oxo (═O); hydroxy;

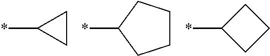

*—NH₂, *—NH—C(O)CH₃, *—NH—SO₂—CH₃, *NH—C(O)—O—C(CH₃)₃,

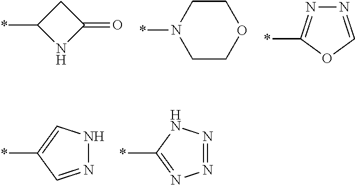

—SO₂—CH₃, *—SO₂—N(CH₃)₂, *—SO₂—NH₂, *O—CH₂—CH₃; *—O—(CH₂)₂—CH₃; *—O—CF₃;

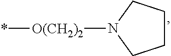

*—OCH₂-Cyclopropyl; *—OCH₃; *—O(CH₂)₃—CH₃; *—OCH₂-Phenyl; *—O-Phenyl, *—(CH₂)—OH, *—(CH₂)₂—OH, *—(CH₂)—O—CH₃, *—(CH₂)—O—CH₂—CH₃, *—CH(OH)—CH₂-Phenyl, *—CH(OH)—CH₂—CH₃, *—CH(OH)—(CH₂)₂—CH₃, *—CH(OH)—(C₂)₃—CH₃, *—CH(OH)—CH—(CH₃)₂, *—CH(OH)-Phenyl, *—CH(OH)—CN, *—CH(OH)—CH₂OH, *—CH(OH)—CF₃, *—CH(OH)—(CH₂)₂-Phenyl, *—CH (OH)—C—CH, *—CH(NH₂)—CH₂—COOH, *—CH₂—NH—SO₂—C—H₃, *—CH₂—NH—(CH₂)₃—CH₃, *—CH₂—NH—CH₃, *—CH₂—N(CH₃)₂, *—CH₂—NH—C₂H₅, *—CH(CH₃)—NH₂, *—CH₂—NH₂, *—(CH₂)₂—NH₂, *—CH₂—NH—CH₂-Phenyl, *—CH₂—N(C₂H₅)₂, *—CH₂—NH-Cyclopropyl, *—CH₂—NH-Cyclobutyl, *—CH₂—NH-Cyclopentyl, *—CH₂—NH-Pyridyl, *—CH₂—NH-Phenyl, *—CH₂—NH—(CH₂)₂—OH, *—CH₂—N(CH₃)(CH₂)₂OH, *—CH₂—NH—CH₂—CN, *—CH₂—N(CH₃)—CH₂—CN, *—CH₂—N(CH₃)—CH₂—CF₃, *—CH₂—N(CH₃)—CH₂—CF₂H, *—CH₂—NH—CH₂—CF₂H, *—CH₂—NH—CH₂—CF₃, *—CH₂, —NH—(CH₂)₂—OCH₃,

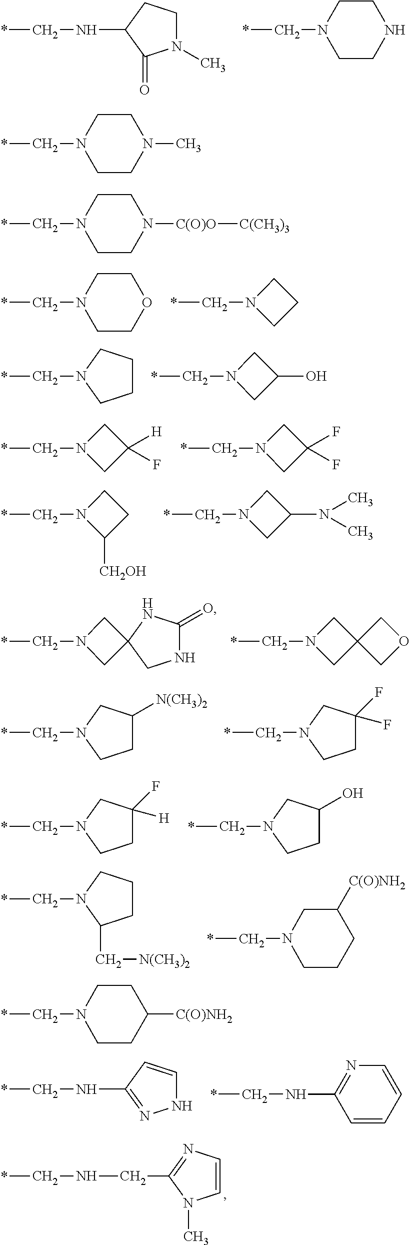

*—CH₂—NH—C(O)—O—C(CH₃)₃, —(CH₂)₂—NH—C(O)—O—C(CH₃)₃, *—CH₂—NH—C(O)—CH₂—OH, —CH₂—NH—C(O)—CH₂—OCH₃, *—CH—(CH₃)—NH—C(O)—O—C(CH₃)₃, —CH₂—NH—C(O)—CH₃,

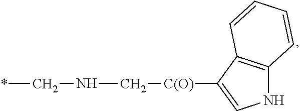

*—CH₂—NH—CH₂—C(O)—NH₂, —CH₂—NH—CH₂—C(O)—N(CH₃)₂, —CH₂—NH—CH₂—C(O)—OCH₃, *—CH₂—NH—CH₂—C(O)—NHCH₃, *—CH₂NH—CH₂—C(O)—NH(CH₂)₂—O—CH₃, —CH₂—NH—CH₂—C(O)—NH—CH₂-Phenyl,

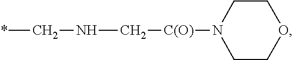

*—CH₂—NH—CH₂—C(O)—NH-Phenyl,

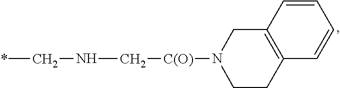

—CH₂—NH—C(O)—CH₂—NH-Phenyl,

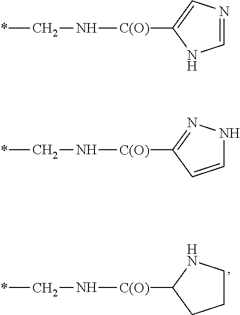

—CH—NH—C(O)—CH₂—NH—CH₂—CF₃,

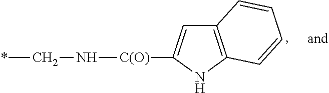

y is 1 or 2 and, k is 1 or 2 and, n is 0 or 1, wherein “*” represents the point of attachment to the remainder of the compound;
wherein the term “fused heterocycloalkyl” as used in this embodiment means a bicyclic, saturated heterocycle with 6, 7, 8, 9 or 10 ring atoms in total, in which the two rings share two adjacent ring atoms, wherein“fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom; the term “bridged heterocycloalkyl” as used in this embodiment means a bicyclic, saturated heterocycle with 7, 8, 9 or 10 ring atoms in total, in which the two rings share two common ring atoms which are not adjacent, wherein “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom; the term “cycloalkenyl” as used in this embodiment means a monovalent, mono- or bicyclic hydrocarbon ring which contains at least one double bond; the term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom; the term “azacycloalkyl” as used in this embodiment means a monocyclic saturated heterocycyl which is attached to the rest of the molecule via the nitrogen atom and which optionally contains one more heteroatom selected from nitrogen and oxygen; the term “heterocycloalkenyl” as used in this embodiment means a monocyclic, unsaturated, non aromatic heterocycle, which contains at least one double bond and one or two identical or different ring heteroatoms from the series: N, O, S; it being possible for said heterocycloalkenyl group to be attached to the rest of the molecule via any one of the carbon atoms or, if present, a nitrogen atom; the term “heterospirocycloalkyl” as used in this embodiment means a bicyclic, saturated heterocycle with 6, 7, 8, 9, 10 or 11 ring atoms in total, in which the two rings share one common ring carbon atom, wherein “heterospirocycloalkyl” contains one, two or three identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

In some embodiments, the SOS inhibitor has the structure of Formula BC or Formula BC′:

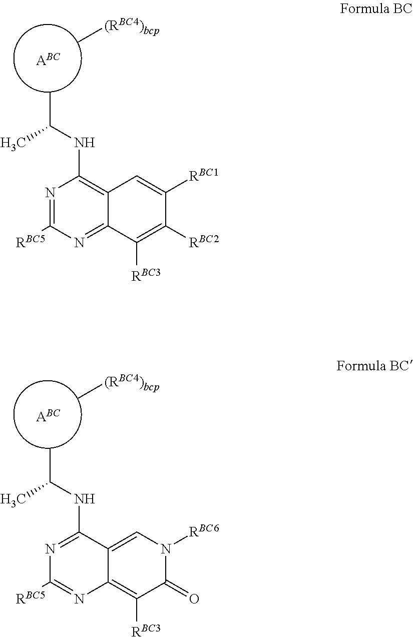

wherein:
ring system A is selected from the group consisting of C₆₋₁₀aryl, 5-10 membered heteroaryl, and 9-10 membered bicyclic heterocyclyl;
R^{BC1} is —O—R^{BCA};
R^{BCA} is selected from the group consisting of C₃₋₁₀cycloalkyl and 3-10 membered heterocyclyl, wherein the C₃₋₁₀cycloalkyl and 3-10 membered heterocyclyl are both optionally substituted by one or more, identical or different R^{BCa1} and/or R^{BCb1};
each R^{BCa1} is independently selected from the group consisting of C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{b1} and/or R^{c1};
each R^{BCb1} is independently selected from the group consisting of —OR^{BCc1}, —NR^{BCc1}, halogen, —CN, —C(O)R^{BCc1}, —C(O)OR^{BCc1}, —C(O)NR^{BCc1}R^{BCc1}, —S(O)₂R^{BCc1}, —S(O)₂NR^{BCc1}R^{BCc1}, —NHC(O)R^{BCc1}, —N(C₁₋₄alkyl)C(O)R^{BCc1} and the bivalent substituent ═O, while ═O may only be a substituent in non-aromatic ring systems;
each R^{BCc1} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl;
or
R^{BC1} is selected from the group consisting of C₃₋₁₀cycloalkyl, C₃₋₁₀cycloalkenyl, C₆₋₁₀aryl, 3-10 membered heterocyclyl and 5-10 membered heteroaryl, wherein the C₃₋₁₀cycloalkyl, C₃₋₁₀cycloalkenyl, C₆₋₁₀aryl, 3-10 membered heterocyclyl and 5-10 membered heteroaryl are all optionally substituted by one or more, identical or different R^{BCa2} and/or R^{BCb2};
each R^{BCa2} is independently selected from the group consisting of C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{BCb2} and/or R^{BCc2};
each R^{BCc2} is independently selected from the group consisting of —OR^{BCc2}, —NR^{BCc2}, R^{BCc2}, halogen, —CN, —C(O)R^{BCc2}, —C(O)OR^{BCc2}, —C(O)NR^{BCc2}, R^{BCc2}, —OC(O)R^{BCc2}, —S(O)₂R^{BCc2}, —S(O)₂NR^{BCc2}, R^{BCc2}, —NHC(O)R^{BCc2}, —N(C₁₋₄alkyl)C(O)R^{BCc2}, —NHC((O)OR^{BCc2} and the bivalent substituents ═O and ═NH, while ═O and ═NH may only be a substituent in non-aromatic ring systems;
each R^{BCc2} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₅₋₁₀aryl, 3-10 membered heterocyclyl and 5-10 membered heteroaryl are all optionally substituted by one or more, identical or different R^{BCd2} and/or R^{BCe2};
each R^{BCd2} is independently selected from the group consisting of —OR^{BCe2}, —NR^{BCe2}R^{BCe2}, halogen, —CN, —C(O)R^{BCe2}, C(O)OR^{BCe2}, —C(O)NR^{BCe2}R^{BCe2}, —S(O)₂R^{BCe2}, —S(O)₂ NR^{BCe2}R^{BCe2}, —NHC(O)R^{BCe2}, —N(C₁₋₄alkyl)C(O)R^{BCe2} and the bivalent substituent ═O, while ═O may only be a substituent in non-aromatic ring systems;
each R^{BCe2} is independently selected from the group consisting of hydrogen, C₁₋₆alkyl, C₂₋₆alkenyl, C₂₋₆alkynyl, C₃₋₁₀cycloalkyl, C₅₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₅₋₁₀aryl, 3-10 membered heterocyclyl and 5-10 membered heteroaryl are all optionally substituted by one or more, identical or different R^{BCf2} and/or R^{BCg2};
each R^{BCf2} is independently selected from the group consisting of —OR^{g2}, —NR^{BCg2}R^{BCg2}, halogen, —CN, —C(O)R^{BCg2}, —C(O)OR^{BCg2}, —C(O)NR^{BCg2}R^{BCg2}, —S(O)₂R^{BCg2}, —S(O)₂NR^{BCg2}R^{BCg2}, NHC(O)R^{BCg2}, —N(C₁₋₄alkyl)C(O)R^{BCg2} and the bivalent substituent ═O, while ═O may only be a substituent in non-aromatic ring systems;
each R^{BCg2} is independently selected from the group consisting of hydrogen, C₁₋₆alkyl, C₂₋₆alkenyl, C₂₋₆alkynyl, C₁₋₁₀cycloalkyl, C₆₋₁₀aryl, 3-10 membered heterocyclyl and 5-10 membered heteroaryl;
or
R^{BC1} is selected from the group consisting of C₂₋₄alkyl and C₂₋₄alkenyl, wherein the C₂₋₄alkyl and C₂₋₄alkenyl are both substituted with R^{b3};
R^{BCb3} is selected from the group consisting of —C(O)R^{BCc3}, —C(O)OR^{BCc3}, C(O)NR^{BCc3}R^{BCc3}, —C(O)NHOR^{BCc3} and —C(O)N(C₁₋₄alkyl)OR^{BCc3};
each R^{BCc3} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{BCd3} and/or R^{BCe3};
each R^{BCd3} is independently selected from the group consisting of —OR^{BCe3}, —NR^{BCe3}R^{BCe3}, halogen, —CN, —C(O)R^{BCe3}, —C(O)OR^{BCe3}, —C(O)NR^{BCe3}R^{BCe3}, —S(O)₂R^{BCe3}, —S(O)₂ NR^{BCe3}R^{BCe3}, NHC(O)R^{BCe3}, —N(C₁₋₄alkyl)C(O)R^{BCe3} and the bivalent substituent ═O, while ═O may only be a substituent in non-aromatic ring systems;
each R^{BCe3} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl;
R^{BC2} is selected from the group consisting of hydrogen, C₁₋₄alkyl, —O—C₁₋₄alkyl, —NH2, —NH(C₁₋₄alkyl), —N(₁₋₄alkyl)₂ and halogen;
R^{BC3} is selected from the group consisting of hydrogen, C₁₋₄alkyl, —O—C₁₋₄alkyl, —NH2, —NH(C₁₋₄alkyl), —N(C₁₋₄alkyl)₂ and halogen;
each R^{BC4} is independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂—C₁₋₄alkyl and the bivalent substituent ═O, while ═O may only be a substituent in a non-aromatic ring;
R^{BC5} is selected from the group consisting of hydrogen, C₁₋₄alkyl and C₁₋₄haloalkyl;
R^{BC6} is R^{BCa6};
R^{BCa6} as is selected from the group consisting of C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl, wherein the C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl are all optionally substituted by one or more, identical or different R^{BCb6} and/or R^{BCc6};
each R^{BCb6} is independently selected from the group consisting of —OR^{BCc6}, —NR^{BCc6}R^{BCc6}, halogen, —C N, —C(O)R^{BCc6}, —C(O)OR^{BCc6}, —C(O)NR^{BCc6}R^{BCc6}, —S(O)₂R^{BCc6}, —S(O)₂NR^{BCc6}R^{BCc6}, —NHC(O)R^{BCc6}, —N(C₁₋₄alkyl)C(O)R^{BCc6}, —NHC(O)OR^{BCc6} and —N(C₁₋₄alkyl)C(O)OR^{BCc6};
each R^{BCc6} is independently selected from the group consisting of hydrogen, C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl, wherein the C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl are all optionally substituted by one or more, identical or different R^{BCd6} and/or R^{BCe6};
each R^{BCd6} is independently selected from the group consisting of —OR^{BCe6}, —NR^{BCe6}R^{BCe6}, halogen, —CN, —C(O)R^{BCe6}, —C(O)O R^{BCe6}, —C(O)NR^{BCe6}R^{BCe6}, —S(O)₂R^{BCe6}, —S(O)₂NR^{BCe6}R^{BCe6}, —NHC(O)R^{BCe6}, —N(C₁₋₄alkyl)C(O)R^{BCe6}, —NHC(O)OR^{BCe6} and —N(C₁₋₄alkyl)C(O)OR^{BCe61};
each R^{BCe6} is independently selected from the group consisting of hydrogen, C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl; or a pharmaceutically acceptable salt thereof.

In embodiments, the SOS1 inhibitor has the formula (CH) immediately below, or a pharmaceutically acceptable salt or solvate thereof:

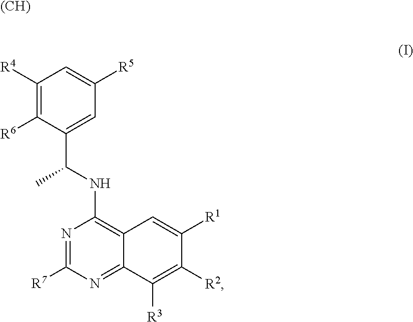

[A0]
R¹ is —O—R^{A};
   R^{A} is selected from the group consisting of C₃₋₁₀cycloalkyl and 3-10 membered heterocyclyl, wherein the C₃₋₁₀cycloalkyl and 3-10 membered heterocyclyl are both optionally substituted by one or more, identical or different R^{a1} and/or R^{b1};
      each R^{a1} is independently selected from the group consisting of C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{b1} and/or R^{c1};
      each R^{c1} is independently selected from the group consisting of —OR^{c1}, —NR^{c1}R^{c1}, halogen, —CN, —C(O)R^{c1}, —C(O)OR^{c1}, —C(O)NR^{c1}, —S(O)₂R^{c1}, —S(O)₂NR^{c1}R^{c1}, —NHC(O)R^{c1}, —N(C₁₋₄alkyl)C(O)R^{c1} and the bivalent substituent ═O, while ═O may only be a substituent in non-aromatic ring systems;
      each R^{c1} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl;
or
R¹ is selected from the group consisting of C₃₋₁₀cycloalkyl, C₃₋₁₀cycloalkenyl, C₆₋₁₀aryl, 3-10 membered heterocyclyl and 5-10 membered heteroaryl, wherein the C₃₋₁₀cycloalkyl, C₃₋₁₀cycloalkenyl, C₆₋₁₀aryl, 3-10 membered heterocyclyl and 5-10 membered heteroaryl are all optionally substituted by one or more, identical or different R^{a2} and/or R^{b2}; each R^{a2} is independently selected from the group consisting of C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{b2} and/or R^{C2};
   each R^{b2} is independently selected from the group consisting of —OR^{C2}, —NR^{C2}R^{C2}, halogen, —CN, —C(O)R^{C2}, —C(O)OR^{C2}, —C(O)NR^{C2}R^{C2}, —OC(O)R^{C2}, —S(O)₂R^{C2}, —S(O)₂NR^{C2}R^{C2}, —NHC(O)R^{C2}, —N(C₁₋₄alkyl)C(O)R^{C2}, —NHC(O)OR^{C2} and the bivalent substituents ═O and ═NH, while ═O and ═NH may only be a substituent in non-aromatic ring systems;
   each R^{C2} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{d2} and/or R^{e2};
      each R^{d2} is independently selected from the group consisting of —OR^{e2}, —NR^{e2}R^{e2}, halogen, —CN, —C(O)R^{e2}, —C(O)OR^{e2}, —C(O)NR^{e2}R^{e2}, —S(O)₂R^{e2}, —S(O)₂NR^{e2}R^{e2}, —NHC(O)R^{e2}, —N(C₁₋₄alkyl)C(O)R^{e2} and the bivalent substituent ═O, while ═O may only be a substituent in non-aromatic ring systems;
         each R^{e2} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{f2} and/or R^{g2};
         each R^{f2} is independently selected from the group consisting of —OR⁹², —NR^{g2}R^{g2}, halogen, —CN, —C(O)R^{g2}, —C(O)OR^{g2}, —C(O)NR^{g2}R^{g2}, —S(O)₂R^{g2}, —S(O)₂NR^{g2}R^{g2}, —NHC(O)R^{g2}, —N(C₁₋₄alkyl)C(O)R^{g2} and the bivalent substituent ═O, while ═O may only be a substituent in non-aromatic ring systems;
         each R^{g2} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl;
or
R¹ is selected from the group consisting of C₂₋₄alkyl and C₂₋₄alkenyl, wherein the C₂₋₄alkyl and C₂₋₄alkenyl are both substituted with R^{b3};
   R^{b3} is selected from the group consisting of —C(O)R^{C3}, —C(O)OR^{C3}, —C(O)NR^{C3}R^{C3}, —C(O)NHOR^{C3} and —C(O)N(C₁₋₄alkyl)OR^{C3};
   each R^{C3} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl, wherein the C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl are all optionally substituted by one or more, identical or different R^{d3} and/or R^{e3};
      each R^{d3} is independently selected from the group consisting of —OR^{e3}, —NR^{e3}R^{e3}, halogen, —CN, —C(O)R^{e3}, —C(O)OR^{e3}, —C(O)NR^{e3}R^{e3}, —S(O)₂R^{e3}, —S(O)₂NR^{e3}R^{e3}, —NHC(O)R^{e3}, —N(C₁₋₄alkyl)C(O)R^{e3} and the bivalent substituent ═O, while ═O may only be a substituent in non-aromatic ring systems;
      each R^{e3} is independently selected from the group consisting of hydrogen, C₁₋₆alkyl, C₂₋₆alkenyl, C₂₋₆alkynyl, C₃₋₁₀cycloalkyl, C₆₋₁₀aryl, 3-10 membered heterocyclyl and 5-10 membered heteroaryl;
[B0]
R² is selected from the group consisting of hydrogen, C₁₋₄alkyl, —O—C₁₋₄alkyl, —NH₂, —NH(C₁₋₄alkyl), —N(C₁₋₄alkyl)₂ and halogen;
[C0]
R³ is selected from the group consisting of hydrogen, C₁₋₄alkyl, —O—C₁₋₄alkyl, —NH₂, —NH(C₁₋₄alkyl), —N(C₁₋₄alkyl)₂ and halogen;
[D0]
R⁴ is selected from the group consisting of C₁₋₄alkyl, C₁₋₄haloalkyl, hydroxy-C₁₋₄alkyl, hydroxy-C₁₋₄haloalkyl, C₃₋₆cycloalkyl, hydroxy-C₃₋₆cycloalkyl, 3-6 membered heterocyclyl, 3-6 membered hydroxy-heterocyclyl, halogen and —SO₂—C₁₋₄alkyl;
[E0]
R⁵ is selected from the group consisting of hydrogen and —NH₂;
[F0]
R⁶ is selected from the group consisting of hydrogen, C₁₋₄alkyl and halogen;
[G0]
R⁷ is selected from the group consisting of C₁₋₄alkyl and C₁₋₄haloalkyl;
or a salt thereof;
cycloalkyl as used in the present embodiment is made up of the subgroups monocyclic hydrocarbon rings, bicyclic hydrocarbon rings and spiro-hydrocarbon rings wherein the systems are saturated. As used in the present embodiment in bicyclic hydrocarbon rings two rings are joined together so that they have at least two carbon atoms in common. As used in the present embodiment in spiro-hydrocarbon rings one carbon atom (spiroatom) belongs to two rings together. As used in the present embodiment cycloalkenyl is also made up of the subgroups monocyclic hydrocarbon rings, bicyclic hydrocarbon rings and spiro-hydrocarbon rings, however, the systems are unsaturated, i.e. there is at least one C═C double bond but no aromatic system. As used in the present embodiment aryl denotes mono-, bi- or tricyclic carbocycles with at least one aromatic carbocycle, wherein the second and/or third rings may also be aromatic or may also be partially saturated. As used in the present embodiment heterocyclyl denotes ring systems, which are derived from the previously defined cycloalkyl, cycloalkenyl and aryl by replacing one or more of the groups —CH₂— independently of one another in the hydrocarbon rings by the groups —O—, —S— or —NH— or by replacing one or more of the groups ═CH— by the group ═N—, wherein at least one carbon atom must be present between two oxygen atoms and between two sulphur atoms or between an oxygen and a sulphur atom and the ring as a whole must have chemically stable. As used in the present embodiment heteroatoms may optionally be present in all the possible oxidation stages (sulphur: sulphoxide —SO—, sulphone —SO₂—; nitrogen: N-oxide). As used in the present embodiment in a heterocyclyl there is no heteroaromatic ring, i.e. no heteroatom is part of an aromatic system. As used in the present embodiment heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. As used in the present embodiment the prerequisite for the presence of heteroaryl is a heteroatom and a heteroaromatic system.

In embodiments, the SOS1 inhibitor has the formula (CI) immediately below, or a pharmaceutically acceptable salt or solvate thereof:

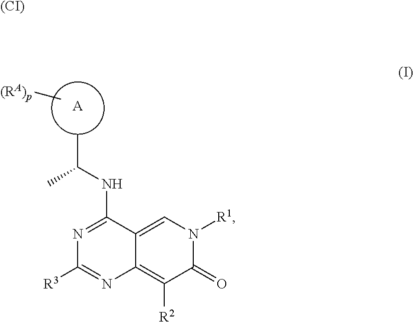

wherein
R¹ is R^{a1};
   R^{a1} is selected from the group consisting of C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl, wherein the C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl are all optionally substituted by one or more, identical or different R^{b1} and/or R^{c1};
      each R^{b1} is independently selected from the group consisting of —OR^{c1}, —NR^{c1}R^{c1}, halogen, —CN, —C(O)R^{c1}, —C(O)OR^{c1}, —C(O)NR^{c1}R^{c1}, —S(O)₂R^{c1}, —S(O)₂NR^{c1}R^{c1}, —NHC(O)R^{c1}, —N(C₁₋₄alkyl)C(O)R^{c1}, —NHC(O)OR^{c1} and —N(C₁₋₄alkyl)C(O)OR^{c1};
         each R^{c1} is independently selected from the group consisting of hydrogen, C₁₋₆alkyl, C₁₋₆haloalkyl, C₂₋₆alkenyl, C₂₋₆ alkynyl, C₃₋₁₀cycloalkyl, C₄₋₁₀cycloalkenyl, 3-10 membered heterocyclyl, C₆₋₁₀aryl and 5-10 membered heteroaryl, wherein the C₁₋₆alkyl, C₁₋₆haloalkyl, C₂₋₆alkenyl, C₂₋₆alkynyl, C₃₋₁₀cycloalkyl, C₄₋₁₀cycloalkenyl, 3-10 membered heterocyclyl, C₆₋₁₀aryl and 5-10 membered heteroaryl are all optionally substituted by one or more, identical or different R^{d1} and/or R^{e1};
         each R^{d1} is independently selected from the group consisting of —OR^{e1}, —NR^{e1}R^{e1}, halogen, —CN, —C(O)R^{e1}, —C(O)OR^{e1}, —C(O)NR^{e1}R^{e1}, —S(O)₂R^{e1}, —S(O)₂NR^{e1}R^{e1}, —NHC(O)R^{e1}, —N(C₁₋₄alkyl)C(O)R^{e1}, —NHC(O)OR^{e1} and —N(C₁₋₄alkyl)C(O)OR^{e1};
         each R^{e1} is independently selected from the group consisting of hydrogen, C₁₋₆alkyl, C₁₋₆haloalkyl, C₂₋₆alkenyl, C₂₋₆ alkynyl, C₃₋₁₀cycloalkyl, C₄₋₁₀cycloalkenyl, 3-10 membered heterocyclyl, C₆₋₁₀aryl and 5-10 membered heteroaryl;
R² is selected from the group consisting of hydrogen, C₁₋₄alkyl, C₃₋₆cycloalkyl, 3-6 membered heterocyclyl and halogen;
R³ is selected from the group consisting of hydrogen, C₁₋₄alkyl and C₁₋₄haloalkyl;
ring system A is selected from the group consisting of C₆₋₁₀aryl, 5-10 membered heteroaryl and 9-10 membered bicyclic heterocyclyl;
p denotes 1, 2 or 3;
each R⁴ is independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂— C₁₋₄alkyl and the bivalent substituent ═O, while ═O may only be a substituent in a non-aromatic ring;
or a salt thereof:
as used in the present embodiment, cycloalkyl is made up of the subgroups monocyclic hydrocarbon rings, bicyclic hydrocarbon rings and spiro-hydrocarbon rings. As used in the present embodiment the systems are saturated. As used in the present embodiment in bicyclic hydrocarbon rings two rings are joined together so that they have at least two carbon atoms in common. As used in the present embodiment in spiro-hydrocarbon rings one carbon atom (spiroatom) belongs to two rings together. As used in the present embodiment cycloalkenyl is also made up of the subgroups monocyclic hydrocarbon rings, bicyclic hydrocarbon rings and spiro-hydrocarbon rings; however, the systems are unsaturated, i.e. there is at least one C═C double bond but no aromatic system; aryl denotes mono-, bi- or tricyclic carbocycles with at least one aromatic carbocycle, wherein the second and/or third rings may also be aromatic or they may also be partially saturated; heterocyclyl denotes ring systems, which are derived from the previously defined cycloalkyl, cycloalkenyl and aryl by replacing one or more of the groups —CH₂— independently of one another in the hydrocarbon rings by the groups —O—, —S— or —NH— or by replacing one or more of the groups ═CH— by the group ═N—, wherein at least one carbon atom must be present between two oxygen atoms and between two sulphur atoms or between an oxygen and a sulphur atom and the ring as a whole must have chemically stable. As used in the present embodiment, heteroatoms may optionally be present in all the possible oxidation stages (sulphur: sulphoxide —SO—, sulphone —SO₂—; nitrogen: N-oxide). As used in the present embodiment, in a heterocyclyl there is no heteroaromatic ring, i.e. no heteroatom is part of an aromatic system.

As used in the present embodiment, heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. As used in the present embodiment, the prerequisite for the presence of heteroaryl is a heteroatom and a heteroaromatic system.

In embodiments, the SOS1 inhibitor is a compound of general formula (CJ):

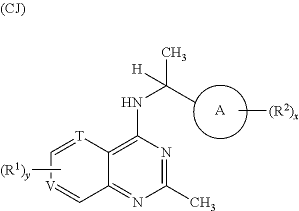

wherein
R¹ is selected from —H, halogen, —OH, —CN, —NO₂, C₁-C₆-alkylsulfanyl,
   —NR^{a}R^{b}, wherein R^{a} and R^{b} are independently selected from —H or C₁-C₆-alkyl,
   C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl, —C(═O)OH,
   —C(═O)OR^{c}, wherein R^{c} stands for C₁-C₆-alkyl, C₃-C₆-alkenyl, C₃-C₆-alkynyl, C₃-C₈-cycloalkyl or C₄-C₈-cycloalkenyl,
   —N═S(═O)(R^{d})R^{e}, wherein R^{d} and R^{e} are independently selected from C₁-C₆-alkyl, C₂-C₆-alkenyl, C₂-C₆-alkynyl, C₃-C₈-cycloalkyl or C₄-C₈-cycloalkenyl,
   —NH—C(O)—C₁-C₆-alkyl, —NH—C(O)—NR^{a}R^{b}, wherein R^{a} and R^{b} are selected independently from a hydrogen atom or a C₁-C₆-alkyl,
   —NH—(CH₂)ₖ—NH—C(O)—C₁-C₆-alkyl, wherein k is 1 or 2,
   —NH—(CH₂)ₗ—R_{f}, wherein 1 is 0, 1 or 2 and R^{f} stands for a 4- to 7-membered heterocycloalkyl, heteroaryl or C₁-C₆-alkylsulfonyl,
   whereby in all foregoing definitions of this embodiment the C₁-C₆-alkyl-, C₁-C₆-alkoxy-, the 4- to 7-membered heterocycloalkyl and the heteroaryl can be optionally substituted, one or two or three times, identically or differently, with a halogen atom, hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy, C₁-C₆-alkylsulfonyl, phenyl, benzyl, heteroaryl, —CH₂-heteroaryl, C₃-C₆-cycloalkoxy, phenyloxy, heteroaryloxy, —NH—C(O)—C₁-C₆-alkyl
      or NR^{a}R^{b}, wherein R^{a} and R^{b} are independently selected from a hydrogen atom or C₁-C₆-alkyl,
      —O—(CH₂)_{z}-phenyl, —O(CH₂)_{z}—C₄-C₇-heterocycloalkyl, —O(CH₂)_{z}-heteroaryl,
         wherein z is 0, 1 or 2, and
         the phenyl, heterocycloalkyl and heteroaryl can optionally be substituted with a group selected from hydroxy, heterocycloalkyl or heterocaclyoalkenyl,
         which both can be substituted with a methyl- and/or oxo-group),

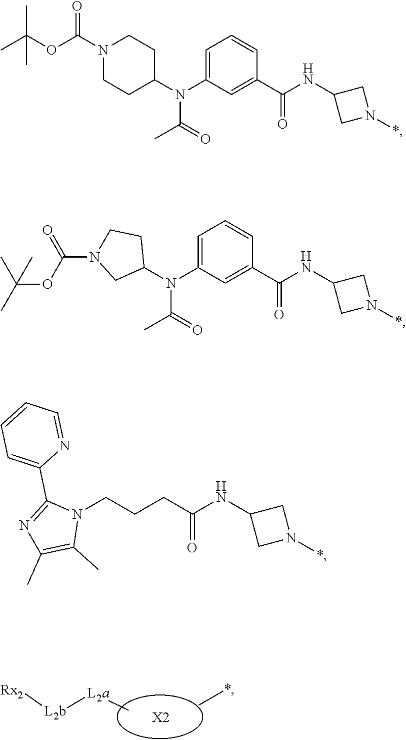

wherein L₂a stands for C(O), L₂b stands for a bond or C₁-C₆—
alkylene, X2 stands for or

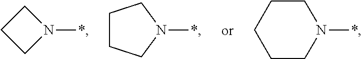

and Rx₂ stands for

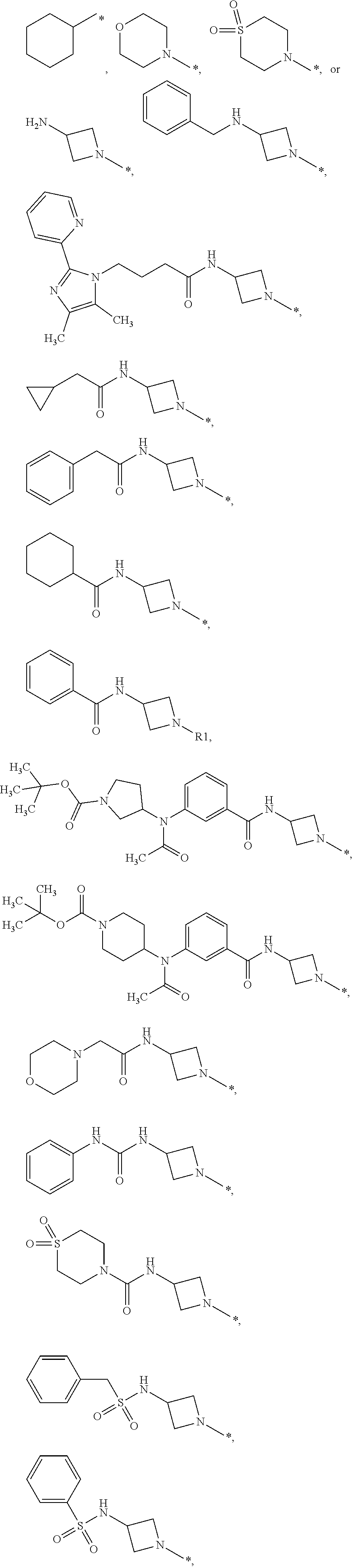

or in which a further R¹ as defined in this embodiment can be directly attached to a first R¹ equaling C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl,
y is 1, 2 or 3; and either both T and V stand for nitrogen or T stands for carbon and V for nitrogen or T for nitrogen and V for carbon;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkynyl, C₁₋₄haloalkyl, hydroxy-C₁₋₄ alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl,
   3-6 membered heterocyclyl substituted with hydroxy, halogen, —NH₂, —SO₂—C₁₋₄alkyl and the bivalent substituent ═O, while ═O may only be a substituent in a non-aromatic ring;
x is 1, 2 or 3;
or a tautomer, an N-oxide, a hydrate, a solvate, or a salt thereof, or a mixture of same;
as used in the present embodiment the term “cycloalkyl” means a saturated, monovalent, mono- or bicyclic hydrocarbon ring. As used in the present embodiment the term “cycloalkenyl” means a monovalent, mono- or bicyclic hydrocarbon ring which contains one double bond. As used in the present embodiment, the term “spirocycloalkyl” means a saturated, monovalent bicyclic hydrocarbon group in which the two rings share one common ring carbon atom, it being possible for said spirocycloalkyl group to be attached to the rest of the molecule via any one of the carbon atoms except the spiro carbon atom. As used in the present embodiment, the term “heterocycloalkyl” means a monocyclic, saturated heterocycle, which contains one or two identical or different ring heteroatoms from the series N, O and S, it being possible for said heterocycloalkyl group to be attached to the rest of the molecule via any one of the carbon atoms or, if present, a nitrogen atom. As used in the present embodiment, the term “membered azacycloalkyl” means a monocyclic saturated heterocycyl which is attached to the rest of the molecule via the nitrogen atom and which optionally contains one more heteroatom selected from nitrogen and oxygen. As used in the present embodiment, the term “fused heterocycloalkyl” means a bicyclic, saturated heterocycle, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom. As used in the present embodiment, the term “bridged heterocycloalkyl” means a bicyclic, saturated heterocycle, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, or, if present, a nitrogen atom. As used in the present embodiment, the term “heteroaryl” means a monovalent, monocyclic, bicyclic or tricyclic aromatic ring, which contains at least one ring heteroatom and optionally one, two or three further ring heteroatoms from the series: N, O and/or S, and which is bound via a ring carbon atom or optionally via a ring nitrogen atom (if allowed by valency).

In embodiments, the SOS1 inhibitor is a compound of general formula (CK):

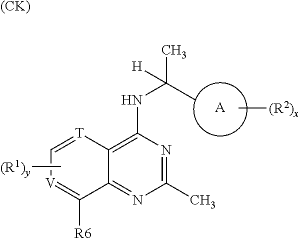

wherein
R¹ is selected from —H, halogen, —OH, —CN, —NO₂, C₁-C₆-alkylsulfanyl,
—NR^{a}R^{b}, wherein R^{a} and R^{b} are independently selected from —H or C₁-C₆-alkyl,
C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl optionally substituted by an oxo-group (═O), fused heterocycloalkyl optionally substituted by an oxo-group (═O), bridged heterocycloalkyl optionally substituted by an oxo-group (═O), phenyl, heteroaryl, C₁-C₆-haloalkyl, —C(═O)OH,
—C(═O)OR^{c}, wherein R^{c} stands for C₁-C₆-alkyl, C₃-C₆-alkenyl, C₃-C₆-alkynyl, C₃-C₈-cycloalkyl or C₄-C₈-cycloalkenyl,
—N═S(═O)(R^{d})R^{e}, wherein R^{d} and R^{e} are independently selected from C₁-C₆-alkyl, C₂-C₆-alkenyl, C₂-C₆-alkynyl, C₃-C₈-cycloalkyl or C₄-C₈-cycloalkenyl,
—NH—C(O)—C₁-C₆-alkyl,
—NH—C(O)—NR^{a}R^{b}, wherein R^{a} and R^{b} are selected independently from a hydrogen atom or a C₁-C₆-alkyl,
—NH—(CH₂)ₖ—NH—C(O)—C₁-C₆-alkyl, wherein k is 1 or 2,
—NH—(CH₂)ₗ—R^{f}, wherein 1 is 0, 1 or 2 and R^{f} stands for a 4- to 7-membered heterocycloalkyl, heteroaryl or C₁-C₆-alkylsulfonyl,
whereby in all foregoing definitions of this embodiment the C₁-C₆-alkyl-, C₁-C₆-alkoxy-, the 4- to 7-membered heterocycloalkyl and the heteroaryl can be optionally substituted, one or two or three times, identically or differently, with a halogen atom, hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy, C₁-C₆-alkylsulfonyl, phenyl, benzyl, heteroaryl, —CH₂-heteroaryl, C₃-C₈-cycloalkoxy, phenyloxy, heteroaryloxy, —NH—C(O)—C₁-C₆-alkyl or
—NR^{a}R^{b}, wherein R^{a} and R^{b} are independently selected from a hydrogen atom or C₁-C₆-alkyl,
—O—(CH₂)_{Z}-phenyl, —O(CH₂)_{Z}—C₄-C₇-heterocycloalkyl, —O(CH₂)_{z}-heteroaryl,
   wherein z is 0, 1 or 2,
   and the phenyl, heterocycloalkyl and heteroaryl can optionally be substituted with a group selected from hydroxy, heterocycloalkyl or heterocycloalkenyl, which both can be substituted with a methyl- and/or oxo-group,

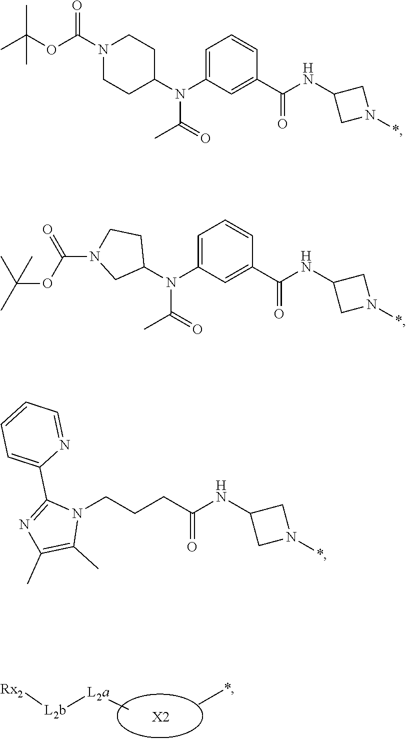

wherein L₂a stands for C(O), L₂b stands for a bond or C₁-C₆-alkylene, X2 stands for

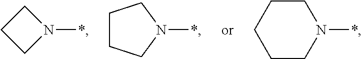

and Rx₂ stands for

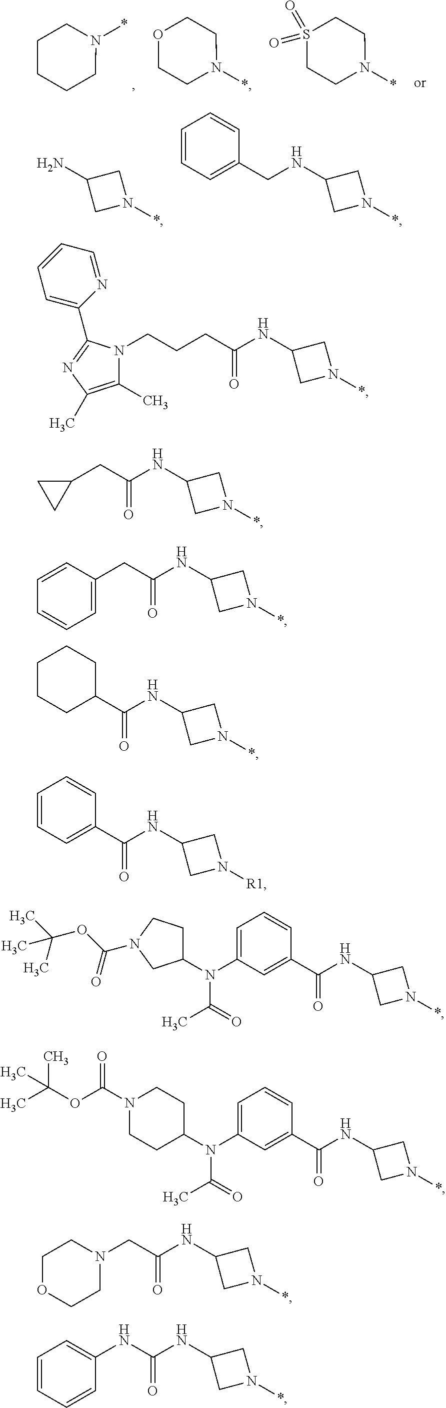

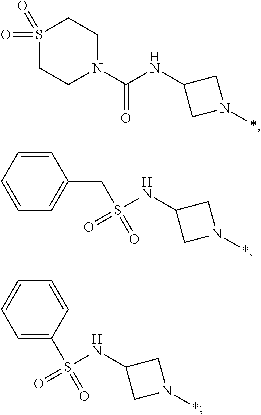

or in which a further R¹ as defined in this paragraph above can be directly attached to a first R¹ equaling C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl,
y is 1, 2 or 3; and either both T and V stand for nitrogen or T stands for carbon and V for nitrogen or T for nitrogen and V for carbon;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkynyl, C₁₋₄haloalkyl, hydroxy-C₁₋₄ alkyl, hydroxy-C₁₋₄haloalkyl, C₃₋₆cycloalkyl, 3-6 membered heterocyclyl, hydroxy-C₃₋₆cycloalkyl, C₁₋₄ haloalkyl substituted with a 3-6 membered heterocyclyl, 3-6 membered heterocyclyl substituted with hydroxy, halogen, —NH₂, —SO₂—C₁₋₄alkyl and the bivalent substituent ═O, while ═O may only be a substituent in a non-aromatic ring;
R⁶ is selected from the group consisting of —H, halogen, C₁₋₄alkyl, C₃-7-cycloalkyl, C₄₋₇ heterocycloalkyl optionally comprising 1 or 2 nitrogen, 1 oxygen or 1 sulphur atom, —O—C₁₋₄alkyl, —NH₂, —NH(C₁₋₄alkyl) or —NH(C₁₋₄alkyl)₂,
x is 1, 2 or 3;
or a tautomer, an N-oxide, a hydrate, a solvate, or a salt thereof, or a mixture of same;
as used in the present embodiment the term “cycloalkyl” means a saturated, monovalent, mono- or bicyclic hydrocarbon ring. As used in the present embodiment the term “cycloalkenyl” means a monovalent, mono- or bicyclic hydrocarbon ring which contains one double bond. As used in the present embodiment, the term “spirocycloalkyl” means a saturated, monovalent bicyclic hydrocarbon group in which the two rings share one common ring carbon atom, it being possible for said spirocycloalkyl group to be attached to the rest of the molecule via any one of the carbon atoms except the spiro carbon atom. As used in the present embodiment, the term “heterocycloalkyl” means a monocyclic, saturated heterocycle, which contains one or two identical or different ring heteroatoms from the series N, O and S, it being possible for said heterocycloalkyl group to be attached to the rest of the molecule via any one of the carbon atoms or, if present, a nitrogen atom. As used in the present embodiment, the term “membered azacycloalkyl” means a monocyclic saturated heterocycyl which is attached to the rest of the molecule via the nitrogen atom and which optionally contains one more heteroatom selected from nitrogen and oxygen. As used in the present embodiment, the term “fused heterocycloalkyl” means a bicyclic, saturated heterocycle, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom. As used in the present embodiment, the term “bridged heterocycloalkyl” means a bicyclic, saturated heterocycle, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, or, if present, a nitrogen atom. As used in the present embodiment, the term “heteroaryl” means a monovalent, monocyclic, bicyclic or tricyclic aromatic ring, which contains at least one ring heteroatom and optionally one, two or three further ring heteroatoms from the series: N, O and/or S, and which is bound via a ring carbon atom or optionally via a ring nitrogen atom (if allowed by valency).
   Alternatively R⁶ of formula (Ia) is selected from the group consisting of —H, —CH₃, —CH(CH₃)₂, —CH₂OH, —CF₃ or —CHF₂.

In embodiments, the SOS1 inhibitor has the formula CK′ or a pharmaceutically acceptable salt, solvate, isomer, prodrug, or tautomer thereof,

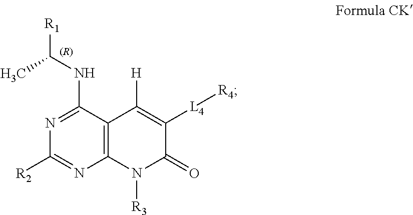

wherein: R₁ is selected from the group consisting of optionally substituted 3-6 membered cycloalkyl, optionally substituted 3-6 membered heterocyclyl, optionally substituted 6-membered aryl, and optionally substituted 5-6 membered heteroaryl; R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocyclyl, and further wherein R₂ₐ is selected from the group consisting of H, C₁₋₆ alkyl, 3-6 membered heterocyclyl, and C₁₋₆ haloalkyl; R₃ is selected from the group consisting of H, C₁₋₃ alkyl, —OR₃ₐ, cyclopropyl, and 3-6 membered heterocyclyl, wherein each of C₁₋₃ alkyl, cyclopropyl, and 3-6 membered heterocyclyl is optionally substituted with R₃ₐ, and further wherein R₃ₐ is selected from the group consisting of C₁₋₃ alkyl, halogen, —OH, or —CN; L₄ is selected from the group consisting of bond, —C(O)—, —C(O)O—, —C(O)NH(CH₂)ₒ—, —NH—, —S—, —S(O)₂—,

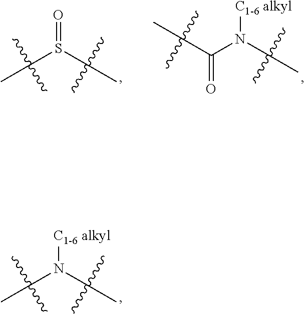

—(CH₂)ₚ—, and —O—; wherein o is 0, 1, or 2; and wherein p is a number from 1 to 6; and R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —R₄ₐ, —OR₄ₐ, —O—C₁₋₆ alkyl-R₄ₐ, ═O, halogen, —C(O)R₄ₐ, —C(OO)R₄ₐ, —C(O)NR_{4b}R_{4c}, —NR_{4b}C(O)R_{4c}, —CN, ═NR₄ₐ, NR_{4b}R_{4c}, —SO₂R₄ₐ, 3-6 membered cycloalkyl optionally substituted with R₄ₐ, 3-7 membered heterocyclyl optionally substituted with R₄ₐ, 6-10 membered aryl optionally substituted with R₄ₐ, or 5-10 membered heteroaryl optionally substituted with R₄ₐ; wherein R₄ₐ is H, C₁₋₆ alkyl, C₁₋₆ haloalkyl, —C(O)R_{4b}, —C(O)NR_{4b}R₄ₐ, ═O, 3-6 membered cycloalkyl, 6-10 membered aryl optionally substituted with —OR_{4b}, —CN, ═N-3-6 membered cycloalkyl, 3-7 membered heterocyclyl, —(CH₂)ᵣOCH₃, or —(CH₂)ᵣOH, wherein r is 1, 2, or 3; wherein each R_{4b} is independently H, C₁₋₆ alkyl; and wherein each R_{4c} is independently H or C₁₋₆ alkyl. In some embodiments, R₁ is

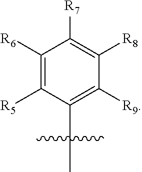

In some embodiments R₁ is a 6-membered heteroaryl having any of the following structures:

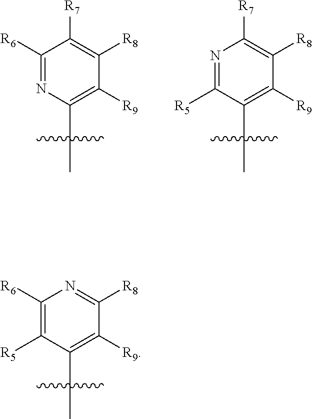

In some embodiments R₁ is a 5-membered heteroaryl having the following structure:

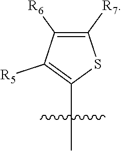

R₅, R₆, R₇, R₈, and R₉ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁, —C(O)R₁₀, —CO₂R₁₀, 6-10 membered aryl, and 5-10 membered heteroaryl, wherein each C₁₋₆ alkyl, C₂₋₆ alkenyl, 4-8 membered cycloalkenyl, C₂₋₆ alkynyl, 3-8 membered cycloalkyl, 3-14 membered heterocyclyl, 6-10 membered aryl, and 5-10 membered heteroaryl is optionally substituted with —OH, C₁₋₆ alkyl optionally substituted with —R₁₀, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁, 3-8 membered cycloalkyl, 3-14 membered heterocyclyl optionally substituted with R₁₀, 6-10 membered aryl, or 5-10 membered heteroaryl, or any two adjacent R⁵, R⁶, R⁷, R⁸, and R⁹ forms an optionally substituted 3-14 membered fused ring; R₁₀, R₁₁, and R₁₂ are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR₁₃, —SR₁₃, halogen, —NR₁₃R₁₄, —NO₂, and —CN; and R₁₃ and R₁₄ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN. In embodiments, R₁ is selected from the group consisting of optionally substituted 3-6 membered cycloalkyl, optionally substituted 3-6 membered heterocycloalkyl, optionally substituted 6-membered aryl, and optionally substituted 5-6 membered heteroaryl; R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocycloalkyl, and further wherein R₂ₐ is selected from the group consisting of H, C₁₋₆ alkyl, 3-6 membered heterocyclyl, and C₁₋₆ haloalkyl; R₃ is selected from the group consisting of H, C₁₋₃ alkyl, cyclopropyl, and 3-6 membered heterocycloalkyl, wherein each of C₁₋₃ alkyl, cyclopropyl, and 3-6 membered heterocycloalkyl is optionally substituted with halogen, —OH, or —CN; L₄ is selected from the group consisting of bond, —C(O)—, —C(O)O—, —C(O)NH(CH₂)ₒ—, —NH—, —S—, —S(O)₂—,

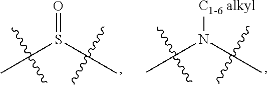

—(CH₂)ₚ—, and —O—; wherein p is a number from 1 to 6; and R₄ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —OR₄ₐ, ═O, halogen, —C(O)R₄ₐ, —C(OO)R₄ₐ, —C(O)NR_{4b}R_{4c}, —CN, —NR_{4b}R_{4c}, 3-6 membered cycloalkyl, 3-7 membered heterocyclyl, 6-10 membered aryl, or 5-10 membered heteroaryl; wherein R₄ₐ is H, C₁₋₆ alkyl, C₁₋₆ haloalkyl, 3-7 membered heterocyclyl, or —(CH₂)ᵣOCH₃, wherein r is 1, 2, or 3; wherein R_{4b} is H or C₁₋₆ alkyl; and wherein R_{4c} is H or C₁₋₆ alkyl. In other embodiments, R₂ is selected from the group consisting of H, C₁₋₆ alkyl, halogen, —NHR₂ₐ, —OR₂ₐ, cyclopropyl, and —CN; wherein C₁₋₆ alkyl is optionally substituted with halogen, —NHR₂ₐ, —OR₂ₐ, or 5-6 membered heterocycloalkyl, and further wherein R₂ₐ is selected from the group consisting of H, C₁₋₆ alkyl, 3-6 membered heterocyclyl, and C₁₋₆ haloalkyl; R₃ is selected from the group consisting of H, C₁₋₃ alkyl, cyclopropyl, and 3-6 membered heterocycloalkyl, wherein each of C₁₋₃ alkyl, cyclopropyl, and 3-6 membered heterocycloalkyl is optionally substituted with halogen, —OH, or —CN; L₄ is a bond; and R⁴ is selected from the group consisting of H, C₁₋₆ alkyl, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl is optionally substituted with C₁₋₆ alkyl, —OR₄ₐ, ═O, halogen, —C(O)R₄ₐ, —C(OO)R₄ₐ, —C(O)NR_{4b}R_{4c}, —CN, —NR_{4b}R_{4c}, 3-6 membered cycloalkyl, 3-7 membered heterocyclyl, 6-10 membered aryl, or 5-10 membered heteroaryl; wherein R₄ₐ is H, C₁₋₆ alkyl, C₁₋₆ haloalkyl, 3-7 membered heterocyclyl, or —(CH₂)ᵣOCH₃, wherein r is 1, 2, or 3; wherein R_{4b} is H or C₁₋₆ alkyl; wherein R_{4c} is H or C₁₋₆ alkyl; R₅, R₆, R₇, R₈, and R₉ are independently selected from the group consisting of H, D, C₁-C₆ alkyl, C₂-C₆ alkenyl, C₄-C₈ cycloalkenyl, C₂-C₆ alkynyl, C₃-C₈ cycloalkyl, —OH, halogen, —NO₂, —CN, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, —C(O)R₁₀, —CO₂R₁₀, aryl, and heteroaryl, wherein each alkyl, alkenyl, cycloalkenyl, alkynyl, cycloalkyl, and aryl is optionally substituted with—OH, C₁-C₆ alkyl optionally substituted with —OH, NR₁₁R₁₂, or heterocyclyl, halogen, —NO₂, oxo, —CN, —R₁₀, —OR₁₀, —NR₁₁R₁₂, —SR₁₀, —S(O)₂NR₁₁R₁₂, —S(O)₂R₁₀, —NR₁₀S(O)₂NR₁₁R₁₂, —NR₁₀S(O)₂R₁₁, —S(O)NR₁₁R₁₂, —S(O)R₁₀, —NR₁₀S(O)NR₁₁R₁₂, —NR₁₀S(O)R₁₁, heterocycle, aryl, or heteroaryl; R₁₀, R₁₁, and R₁₂ are independently, at each occurrence, H, D, C₁-C₆ alkyl, C₂-C₆ alkenyl, C₄-C₈cycloalkenyl, C₂-C₆ alkynyl, C₃-C₈cycloalkyl, 3-14 membered heterocyclyl, —OR₁₃, —SR₁₃, halogen, —NR₁₃R₁₄, —NO₂, or —CN; and R₁₃ and R₁₄ are independently, at each occurrence, H, D, C₁-C₆ alkyl, C₂-C₆ alkenyl, C₄-C₈ cycloalkenyl, C₂-C₆ alkynyl, C₃-C₈ cycloalkyl, 3-14 membered heterocyclyl, wherein each alkyl, alkenyl, cycloalkenyl, alkynyl, cycloalkyl, and heterocyclyl is optionally substituted with—OH, —SH, —NH₂, —NO₂, or —CN. As used in this paragraph, “Cycloalkyl” refers to a single saturated all carbon ring having 3 to 20 annular carbon atoms (i.e., C₃-C₂₀ cycloalkyl). In certain embodiments, the cycloalkyl group is either monocyclic (“monocyclic cycloalkyl”) or contains a fused, bridged or spiro ring system such as a bicyclic system (“bicyclic cycloalkyl”) and can be saturated. As used in this paragraph “Cycloalkyl” includes ring systems where the cycloalkyl ring, as defined above, is fused with one or more cycloalkyl, cycloalkenyl, heterocyclyl, aryl or heteroaryl groups, wherein the point of attachment is on a cycloalkyl ring, and, in such instances, the number of carbon atoms recited continues to designate the number of carbons in the cycloalkyl ring containing the point of attachment. As used in this paragraph, the term “cycloalkenyl” may refer to a partially saturated, monocyclic, fused or spiro polycyclic, all carbon ring having from 3 to 18 carbon atoms per ring and contains at least one double bond. As used in this paragraph “Cycloalkenyl” includes ring systems where the cycloalkenyl ring, as defined above, is fused with one or more cycloalkyl, cycloalkenyl, heterocyclyl, aryl or heteroaryl groups, wherein the point of attachment is on a cycloalkenyl ring, and, in such instances, the number of carbon atoms recited continues to designate the number of carbons in the cycloalkenyl ring containing the point of attachment. As used in this paragraph the term “aryl” refers to a single all carbon aromatic ring or a multiple condensed all carbon ring system wherein at least one of the rings is aromatic. As used in this paragraph Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., cycloalkyl). As used in this paragraph “Aryl” includes ring systems where the aryl ring, as defined above, is fused with one or more cycloalkyl, cycloalkenyl, heterocyclyl, aryl or heteroaryl groups, and wherein the point of attachment is on an aryl ring, and, in such instances, the number of carbon atoms recited continues to designate the number of carbon atoms in the aryl ring containing the point of attachment. As used in this paragraph “Heterocyclyl” refers to a single saturated or partially unsaturated non-aromatic ring or a non-aromatic multiple ring system (including fused and spiro polycyclic) that has at least one heteroatom in the ring (at least one annular heteroatom selected from oxygen, nitrogen, phosphorus, and sulfur). As used in this paragraph the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen, phosphorus, and sulfur in the ring. As used in this paragraph the term also includes single saturated or partially unsaturated rings (e.g., 5, 6, 7, 8, 9, or 10-membered rings) having from about 4 to 9 annular carbon atoms and from about 1 to 3 annular heteroatoms selected from the group consisting of oxygen, nitrogen, phosphorus, and sulfur in the ring. As used in this paragraph “Heterocyclyl” includes ring systems where the heterocyclyl ring, as defined above, is fused with one or more cycloalkyl, cycloalkenyl, heterocyclyl, aryl or heteroaryl groups, wherein the point of attachment is on a heterocyclic ring, and, in such instances, the number of ring members recited continues to designate the number of annular atoms in the heterocyclic ring containing the point of attachment. As used in this paragraph the term “heteroaryl” refers to a single aromatic ring that has at least one atom other than carbon in the ring, wherein the atom is selected from the group consisting of oxygen, nitrogen and sulfur; the term also includes multiple condensed ring systems that have at least one such aromatic ring. As used in this paragraph, the term includes single heteroaryl rings of from about 1 to 10 annular carbon atoms and about 1-5 annular heteroatoms selected from the group consisting of oxygen, nitrogen and sulfur in the rings. As used in this paragraph the sulfur and nitrogen atoms may also be present in an oxidized form provided the ring is aromatic. As used in this paragraph “Heteroaryl” includes ring systems where the heteroaryl ring, as defined above, is fused with one or more cycloalkyl, cycloalkenyl, heterocyclyl, aryl or heteroaryl groups, wherein the point of attachment is on a heteroaryl ring, and, in such instances, the number of ring members continues to designate the number of ring members in the heteroaryl ring containing the point of attachment.

SHP2 Inhibitors

In embodiments, the SHP2 inhibitor has the formula (CL) immediately below, or a pharmaceutically acceptable salt or solvate thereof:

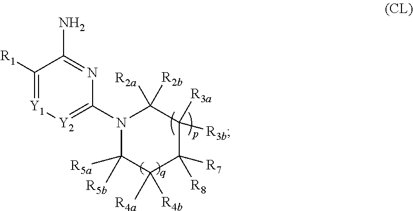

in which:
p is selected from 0 and 1;
q is selected from 0 and 1;
Y₁ is selected from CH and N;
Y₂ is selected from CR₆ and N:
R₁ is —XR₁ₐ;
   wherein R₁ₐ is selected from C₆₋₁₀aryl, C₃₋₆cycloakyl, C₃₋₈Cycloalkenyl and a 5-9 member heteroaryl group containing from 1 to 4 heteroatoms or groups independently selected from N, C(O), O and S;
      wherein said aryl or heteroaryl of R₁ₐ is substituted with 1 to 5 R₉ groups independently selected from halo, amino, hydroxy, N₃, C₁₋₄alkyl, dimethyl-amino, hydroxy-substituted-C₁₋₄alkyl, halo-substituted-C₁₋₄alkyl, amino-substituted-C₁₋₄alkyl, —C(O)OR₁₀ and —NHC(O)R₁₀; and
   X is selected from a bond, S, S(O)ₘ, O, C(O), COR₁₁, CR₁₀ₐR_{10b}, NR₁₁;
      wherein m is selected from 0, 1 and 2;
      each R₁₀ₐ and R_{10b} is independently selected from halo and C₁₋₄alkyl; and R₁₁ is selected from hydrogen and C₁₋₄alkyl;
R₂ₐ and R_{2b} are independently selected from hydrogen, C₁₋₄alkyl, C₁₋₄alkoxy, amino, hydroxy, C₃₋₈cycloalkyl and C₁₋₄alkyl-amino;
R₃ₐ and R_{3b} are independently selected from hydrogen, halo, carbonyl, C₁₋₄alkyl, C₁₋₄alkoxy, amino, hydroxy, C₃₋₈cycloalkyl and C₁₋₄ alkyl-amino;
R₄ₐ and R_{4b} are independently selected from hydrogen, halo, carbonyl, C₁₋₄alkyl, C₁₋₄alkoxy, amino, hydroxy, C₃₋₈cycloalkyl and C₁₋₄alkyl-amino;
R₅ₐ and R_{5b} are independently selected from hydrogen, carbonyl, C₁₋₄alkyl, C₁₋₄alkoxy, amino, hydroxy, C₃₋₈cycloalkyl and C₁₋₄alkyl amino;
wherein any two groups selected from R₂ₐ, R_{2b}, R₃ₐ, R_{3b}, R₄ₐ, R_{4b}, R₅ₐ, R_{5b} and R₇ can form a 5 to 6 member unsaturated or partially saturated ring;
R₆ is selected from hydrogen, halo, cyano, C₁₋₄alkyl, C₁₋₄alkoxy, amino-carbonyl, halo-substituted Ct4alkyl, halo-substituted C₁₋₄alkoxy, hydroxy-substituted C₁₋₄alkyl, amino-substituted C₁₋₄alkyl, —S(O)₁₋₂R₆ₐ, —C(S)R₆ₐ, —C(O)NR₆ₐR_{6b}, —C(NH)NR₆ₐR_{6b} and —NR₆ₐC(O)R_{6b};
   wherein R₆ₐ and R_{6b} are independently selected from hydrogen and C₁₋₄alkyl;
R₇ and R₈ together with the carbon atom to which they are both attached form a 3 to 7 member saturated or partially unsaturated ring that can optionally contain 1 to 3 heteroatoms or groups independently selected from N, C(O), O and S(O)ₘ;
   wherein m is selected from 0, 1 and 2;
wherein said saturated ring formed by R₇ and R₈ can be unsubstituted or substituted with 1 to 3 groups independently selected from amino, hydroxy, methoxy, halo, methyl, methyl-amino and isobutyryloxy;
as used in the present embodiment, “Aryl” means a monocyclic or fused bicyclic aromatic ring assembly containing six to ten ring carbon atoms. As used in the present embodiment, “Heteroaryl” is as defined for aryl in this embodiment where one or more of the ring members is a heteroatom. As used in the present embodiment, “Cycloalkyl” means a saturated or partially unsaturated, monocyclic, fused bicyclic or bridged polycyclic ring assembly. As used in the present embodiment, “Heterocycloalkyl” means cycloalkyl, as defined in this embodiment, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group.

In embodiments, the SHP2 inhibitor is

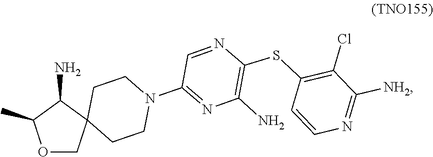 [SMILES: C[C@@H]1OCC2(CCN(c3cnc(Sc4ccnc(N)c4Cl)c(N)n3)CC2)[C@@H]1N]

or a pharmaceutically acceptable salt or solvate thereof. In embodiments, the SHP2 inhibitor is (3S,4S)-8-(6-amino-5-((2-amino-3-chloropyridin-4-yl)thio)pyrazin-2-yl)-3-methyl-2-oxa-8-azaspiro[4.5]decan-4-amine, or a pharmaceutically acceptable salt or solvate thereof.

In embodiments, the SHP2 inhibitor has the formula (CM) immediately below, or a pharmaceutically acceptable salt or solvate thereof:

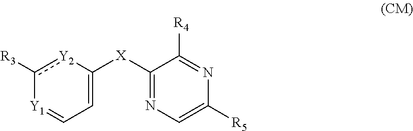

X is absent, O, S, SO, S(O)₂, C(O), C(O)R₁₁, CR₁₁R₁₂, or —NR₁₁; and each R₁₃ and R₁₂ is independently —H, halogen, —NH₂, —CN, —OH, —NO₂, carbonyl, ═O, oxo, carboxyl, substituted or unsubstituted C₁₋₆alkoxy, or substituted or unsubstituted C₁₋₆alkyl;
Y₁ is N or CR₁;
Y₂ is N or CR₂;
   each R₁ and R₂ is independently —H, halogen, —CN, —OH, —NH₂, —N₃, —NO₂, substituted or unsubstituted C₁₋₆alkoxy, or substituted or unsubstituted C₁₋₆alkyl; or
   R₁ combines with R₃, or R₂ combines with R₃, to form a 5-10 member heteroaryl, 5-10 member carbocyclic or 5-10 member heterocyclic ring, wherein each of the ring systems is independently optionally substituted with halogen, —CN, —OH, —NR₈R₉, —N₃, —NO₂, carbonyl, ═O, oxo, substituted or unsubstituted C₁₋₆alkyl, substituted or unsubstituted C₁₋₆alkoxy, or C(O)R₈; or
R₃ is —H, halogen, —CN, —OH, —N₃, —NO₂, —NR₈R₉, —N(R₈)(CH₂)ₚNR₈R₉, —N(R₈)(CH₂)ₚR₈, —N(R)GₚR₈, —N(R₈)GₚNR₈R₉, —N(R₈)(C═O)_{q}R₈, —N(R₈)(C═O)_{q}NR₈R₉, N(R₈) (C═O)GₚR₈R₉, —N(R₈) (C═O)ₚGₚNR₈R₉, —N(R₈)(C═O)_{q}Gₚ(C═O)_{q}R₈, —N(R₈)(C═O)_{q}Gₚ(C═O)_{q}NR₈R₉, —N(R₈)(C═O)_{q}N(R₈)(C═O)_{q}R₈, —N(R₈) (C═O)_{q}N(R₈)(C═O)_{q}NR₈R₉, —N(R₈)(C═O)_{q}N(R₈)G_{q}(C═O)R, —N(R₈)(C═O)_{q}N(R₈)Gₚ(C═O)₄NR₈R₉, C(O)_{q}R₈, C(O)OR₈, C(O)NH₂, C(O)NHR₈, C(O)NR₈R₉, C₁₋₆alkyl, C₆₋₁₀aryl, arylalkyl, alkoxy, heteroaryl, heterocyclic, or carbocyclic; and each of which may be optionally substituted; and each p and q is independently 0, 1, 2 or 3;
each G is independently C₆₋₁₀aryl, C₃₋₆carbocyclic or C₅₋₁₀heteroaryl; and each of which may be optionally substituted;
R₄ is —H, halogen, —CN, —Ol, —NR₈R₉, —N₃, —NO₂, substituted or unsubstituted C₁₋₆alkoxy, substituted or unsubstituted C₁₋₆alkyl, C₅₋₁₀ heterocyclic or C₅₋₁₀carbocyclic; wherein each of the ring systems is independently optionally substituted with halogen, —CN, —OH, —NO₂, carbonyl, ═O, oxo, substituted or unsubstituted C₁₋₆alkyl, substituted or unsubstituted C₁₋₆alkoxy, —NR₈R₉, or —CH₂NR₈R₉;
R₅ is —H, halogen, —CN, —OH, —NR₈R₉, —N₃, —NO₂, C₁₋₆alkyl, C₁₋₆alkoxy, C₆₋₁₀aryl, C₆₋₁₀arylalkyl, C₆₋₁₀heteroaryl, C₅₋₁₀heterocyclic or C₅₋₁₈carbocyclic; and each of which is independently optionally substituted (e.g, with NH₂);
   each R₈ and R₉ is independently —H, halogen, —CN, —OH, —N₃, —NO₂, C₁₋₆alkyl, C₁₋₆alkoxy, C₂₋₆alkenyl, NH(C₁₋₆alkyl), N(C₁₋₆alkyl)₂, C₅₋₁₀heterocyclic or C₅₋₁₀carbocyclic; and each of which may be independently optionally substituted;
as used in the present embodiment, the term “aryl”, unless otherwise indicated, refers to an unsubstituted or substituted mono- or polycyclic aromatic ring system containing carbon ring atoms. As used in the present embodiment, the term “heterocyclic”, unless otherwise indicated, refers to unsubstituted and substituted mono- or polycyclic non-aromatic ring system containing one or more heteroatoms. As used in the present embodiment, the term “heteroaryl” unless otherwise indicated, represents an aromatic ring system containing carbon (s) and at least one heteroatom. As used in the present embodiment, heteroaryl may be monocyclic or polycyclic, substituted or unsubstituted. As used in the present embodiment, the term “cycloalkyl” refers to a substituted or unsubstituted monocyclic, bicyclic or polycyclic non-aromatic saturated ring, which optionally includes an alkylene linker through which the cycloalkyl may be attached.
In embodiments, the compound is

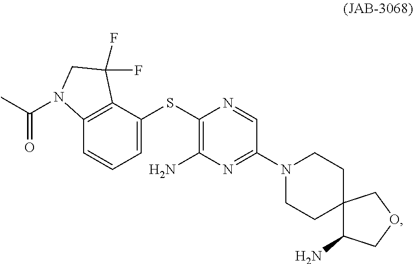 [SMILES: CC(=O)N1CC(F)(F)c2c(Sc3ncc(N4CCC5(CC4)COC[C@H]5N)nc3N)cccc21]

or a pharmaceutically acceptable salt or solvate thereof.

In embodiments, the SHP2 inhibitor has the formula (CN) immediately below, or a pharmaceutically acceptable salt or solvate thereof:

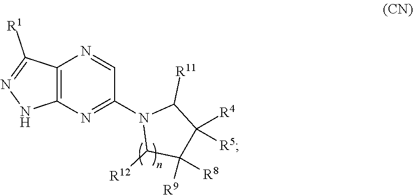

wherein
R¹ is a 5-10 membered monocyclic or bicyclic aryl or heteroaryl, which is optionally substituted with one or more substituents selected from the group consisting of
   —R¹⁰, —OR¹⁰, SR¹⁰, —N(R¹⁰)₂, —OSO₂R¹⁰, —SO₂R¹⁰, —C(O)N(R¹⁰)₂, halogen, or nitrile, wherein each R¹⁰ is, independently, H, —(C₁-C₆)alkyl, —(C₁-C₆)haloalkyl, or —(C₁-C₆)heterocycloalkyl;
each of R⁴ and R⁵ is, independently, H, —OH, —(C₁-C₆)alkyl, —O(C₁-C₆)alkyl, —(C₁-C₆)alkyl-O—R⁶, —C(O)NH₂, —N(R⁶)₂, halogen, —(C₁-C₆)alkyl-N(R⁶)₂, or nitrile, wherein said —(C₁-C₆)alkyl, —O(C₁-C₆)alkyl, —(C₁-C₆)alkyl-O—R⁶, or —(C₁-C₆)alkyl-N(R⁶)₂ is optionally substituted with
   one or more substituents selected from the group consisting of —OH, —N(R⁶)₂, oxo, and halogen, wherein each R⁶ is independently H or —(C₁-C₆)alkyl;
or R⁴ and R⁵, taken together with the atoms to which they are attached, form a 3-7 membered carbocyclic or heterocyclic ring, which ring is optionally substituted with —OH, —(C₁-C₆)alkyl, —O(C₁-C₆)alkyl, —N(R⁶)₂, halogen, oxo, or nitrile;
or R⁴ and R⁸, taken together with the atoms to which they are attached, form a 4-7 membered carbocyclic or heterocyclic ring, which ring is optionally substituted with —OH, —(C₁-C₆)alkyl, —O(C₁-C₆)alkyl, —N(R⁶)₂, halogen, oxo, or nitrile;
or R⁴ is a bond, and R⁴ and R⁸, taken together with the atoms to which they are attached, form a 3-membered carbocyclic or heterocyclic ring, which ring is optionally substituted with —OH, —(C₁-C₆)alkyl, —O(C₁-C₆)alkyl, —N(R⁶)₂, halogen, oxo, or nitrile;
each of R¹¹ and R¹² is, independently, H, —OH, —(C₁-C₆)alkyl, —O(C₁-C₆)alkyl, —(C₁-C₆)alkyl-O—R⁶, —C(O)NH₂, —N(R^{p})₂, halogen, —(C₁-C₆)alkyl-N(R⁶)₂, —CO₂H, or nitrile, wherein said —(C₁-C₆)alkyl, —O(C₁-C₆)alkyl, —(C₁—C)alkyl-O—R⁶, or —(C₁-C₆)alkyl- N(R⁶)₂ is optionally substituted with one or more substituents selected from the group consisting of —OH, —N(R⁶)₂, and halogen;
or R¹¹ and R¹², taken together with the atoms to which they are attached, form a 5-7 membered heterocyclic ring;
or R⁴ and R¹², taken together with the atoms to which they are attached, form a 5-7 membered carbocyclic or heterocyclic ring;
or R⁸ and R¹¹, taken together with the atoms to which they are attached, form a 5-7 membered carbocyclic or heterocyclic ring;
each of R⁸ and R⁹ is, independently, H, —(C₁-C₆)alkyl, —(C₁-C₆)alkyl-N(R⁶)₂, —OR⁶, —(C₁-C₆)alkyl-O—R⁶, —C(O)NH₂, —N(R⁶)₂, halogen, or nitrile;
and each of m and n is, independently, 0, 1, 2, or 3, with m+n being no more than 4;

as used in the present embodiment, the term “alkyl”, unless otherwise indicated, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. In embodiments, the SHP2 inhibitor has the formula (CO) immediately below, or a pharmaceutically acceptable salt or solvate thereof:

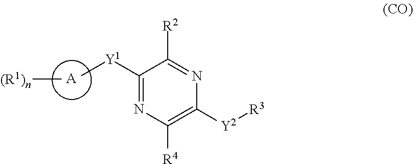

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S— or a direct bond;
Y² is —NR^{a}—, —(CR^{a}₂)ₘ—, —C(O)—, —C(R^{a})₂NH, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₆cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁶, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₆cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR_{S(O)R}⁶, heterocycle, aryl, or heteroaryl; and wherein the heterocyclyl or heteroaryl is not attached via a nitrogen atom;
R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —C₂N, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;
R⁴ is independently —H, -D, or —C₁-C₆alkyl, wherein each alkyl is optionally substituted with one or more —OH, —NH₂, halogen, or oxo; or
   R^{a} and R⁴ together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;
   R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;
      R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CP) immediately below, or a pharmaceutically acceptable salt or solvate thereof:

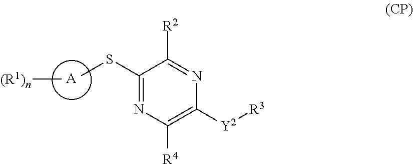

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₇cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NWS(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, —H, -D, —OH, —C₃-C₆cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁷, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;
R⁴ is independently —H, -D, or —C₁-C₆alkyl, wherein each alkyl is optionally substituted with one or more —OH, —NH₂, halogen, or oxo; or
   R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR¹, —SR⁷, halogen, —NR⁷R⁸, —NO₂, or —CN;
   R⁷ and R⁸ are independently, at each occurrence, —H, -D, —C₃-C₈alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2-group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CQ) immediately below, or a pharmaceutically acceptable salt or solvate thereof:

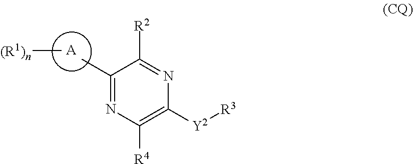

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂RV, —NR⁶S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;
R⁴ is independently —H, -D, or —C₁-C₆alkyl, wherein each alkyl is optionally substituted with one or more —OH—, —NH₂, halogen, or oxo; or
   R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2-group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CR) immediately below, or a pharmaceutically acceptable salt or solvate thereof:

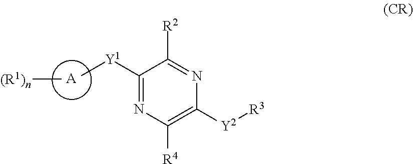

and pharmaceutically acceptable salts, prodrugs, solvates, hydrates, tautomers, or isomers thereof, wherein:
A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;
Y¹ is —S—, a direct bond, —NH—, —S(O)₂—, —S(O)₂NH— —C(═CH₂)—, —CH—, or —S(O)—
Y² is —NR^{a}—, wherein the bond on the left side of Y², as drawn, is bound to the pyrazine ring and the bond on the right side of the Y² moiety, as drawn, is bound to R³;
   R^{a} and R⁴, together with the atom or atoms to which they are attached, are combined to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)2- in the heterocycle;
R¹ is independently, at each occurrence, —H, -D, —C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R, —NR⁵S(O)NR⁵R⁶, —NR⁵S(O)R⁶, —C(O)R⁵, —CO₂R₅, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R₁, —S(O)₂R⁵, —NR⁵S(O)₂NR⁵R⁶, —NR⁵S(O)₂R⁶, —S(O)NR⁵R⁶, —S(O)R_{, —NR}⁵S(O)NR⁵R⁶, —NR⁶S(O)R⁶, heterocycle, aryl, or heteroaryl;
R² is independently —NH₂, —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S. P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{b} is independently, at each occurrence, —H, -D. —OH, —C₁-C₆alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)n-aryl is optionally substituted with one or more —OH, halogen, —NO₂, oxo, —CN, —R_{, —OR}⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;
R³ is independently —H, —C₁-C₆alkyl, a 3- to 12-membered monocyclic or polycyclic heterocycle, a 5- to 12-membered spiroheterocycle, C₃-C₈cycloalkyl, or —(CH₂)ₙ—R^{b}, wherein each alkyl, spiroheterocycle, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SIR, halogen, —NR⁷R⁸, —NO₂, —CF₃, or —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the Formula (CS) below, or a pharmaceutically acceptable salt or solvate thereof:

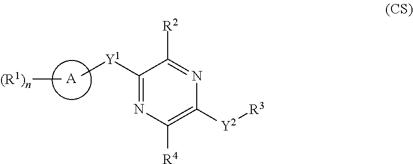

and pharmaceutically acceptable salts, prodrugs, solvates, hydrates, tautomers, or isomers thereof, wherein:
A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;
Y¹ is —S—, a direct bond, —NH—, —S(O)₂—, —S(O)₂NH— —C(═CH₂)—, —CH—, or —S(O)—
Y² is —NR^{a}—, wherein the bond on the left side of Y², as drawn, is bound to the pyrazine ring and the bond on the right side of the Y² moiety, as drawn, is bound to R³;
R³ is combined with R^{a} to form a 3- to 12-membered polycyclic heterocycle or a 5- to 12-membered spiroheterocycle, wherein each heterocycle or spiroheterocycle is optionally substituted with one or more —C₁-C₆alkyl, halogen, —OH, —OR^{b}, —NH₂, —NHR, heteroaryl, heterocyclyl, —(CH₂)ₙNH₂, —(CH₂)ₙOH, —COOR^{b}, —CONHR^{b}, —CONH(CH₂)ₙCOOR^{b}, —NHCOOR^{b}, —CF₃, —CHF₂, —CH₂F, or ═O;
R¹ is independently, at each occurrence, —H, -D, —C₁-C₆alkyl, —C₂-C₆alkenyl, —C₄-C₈cycloalkenyl, —C₂-C₆alkynyl, —C₃-C₆cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R, —NR⁵S(O)NR⁵R⁶, —NR⁵S(O)R⁶, —C(O)R⁵, —CO₂R₅, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR′, —S(O)₂NR⁵R⁶, —S(O)₂R⁵, —NR S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —NH₂, —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R′, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{b} is independently, at each occurrence, —H, -D. —OH, —C₁-C₆alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)˜OH, —C₁-C₆alkyl, —CF₃, —CHF₂, or —CH₂F;
R⁴ is independently —H, -D, —C₁-C₆alkyl, —C₁-C₆haloalkyl, —C₁-C₆hydroxyalkyl, —CF₂OH, —CHFOH, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CT) immediately below, or a pharmaceutically acceptable salt or solvate thereof:

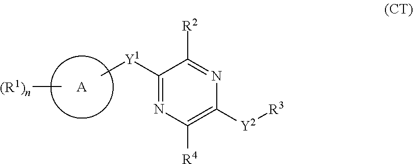

and pharmaceutically acceptable salts, prodrugs, solvates, hydrates, tautomers, and isomers thereof, wherein:
A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;
Y¹ is —S—, a direct bond, —NH—, —S(O)₂—, —S(O)₂NH—, —C(═CH₂)—, —CH—, or —S(O)—
Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y2, as drawn, is bound to the pyrazine ring and the bond on the right side of the Y² moiety, as drawn, is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₆cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)R⁵R⁶, —S(O)R⁵, —NR⁵S(O)NR⁵R⁶, —NR⁵S(O)R⁶, —C(O)R⁵, —CO₂R₅, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁶, —NR⁵S(O)NR⁵R⁶, —NR⁵S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃- C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁵, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, —H, -D, —OH, —C₃-C₈cycloalkyl, —C₁-C₆alkyl, 3- to 12-membered heterocyclyl, or —(CH₂)ₙ-aryl, wherein each alkyl or cycloalkyl is optionally substituted with one or more —N—, or wherein 2 R′, together with the carbon atom to which they are both attached, can combine to form a 3- to 8-membered cycloalkyl;
   R^{b} is independently, at each occurrence, —H, -D, —OH, —C₁—C alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)ₙ-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;
R³ is independently —H, —C₁-C₆alkyl, a 3- to 12-membered monocyclic or polycyclic heterocycle, a 5- to 12-membered spiroheterocycle, C₃-C₆cycloalkyl, or —(CH₂)ₙ—R^{b}, wherein each alkyl, spiroheterocycle, heterocycle, or cycloalkyl is optionally substituted with one or more —C₁-C₆alkyl, —OH, —NH₂, —OR^{b}, —NHR, —(C₁₋₂)ₙOH, heterocyclyl, or spiroheterocyclyl; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, halogen, —OH, —OR^{b}, —NH₂—, —NHR^{b}, heteroaryl, heterocyclyl, —(CH₂)ₙNH₂—, —(CH₂)ₙOH, —COOR^{b}, —CONHR^{b}, —CONH(CH₂)nCOOR^{b}, —NHCOOR^{b}, —CF₃, —CHF₂, —CH₂F, or ═O;
R⁴ is independently —H, -D, —C₁-C₆alkyl, —C₁-C₆haloalkyl, —C₁-C₆hydroxyalkyl —CF₂OH, —CHFOH, —NH—NHR⁵, NH—OR⁵, —O—NR⁵R⁶, —NHR⁵, —OR⁵, —NHC(O)R⁵, —NHC(O) NR⁵, —NHS(O)₂R⁵, —NHS(O)₂NR⁵R⁶, —S(O)₂OH, —C(O)OR⁵, —NH(CH₂)ₙOH, —C(O)NH(C H₂) OH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂—, or halogen; or
   R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or—CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR^{b}, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CU) immediately below, or a pharmaceutically acceptable salt or solvate thereof:

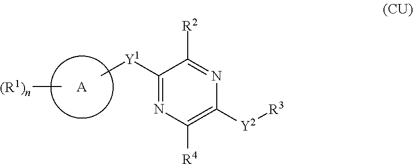

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S— or a direct bond;
Y² is —NR^{a}—, —(CR^{a}₂)ₘ—, —C(O)—, —C(R^{a})₂NH— —(CR^{a}₂)ₘO—, —C(O)N(R^{a})—, —N(R^{a})C(O)—, —S(O)₂N(R^{a})—, —N(R^{a})S(O)₂, —N(R^{a})C(O)N(R^{a})—, N(R^{a})C(S)N(R^{a})—, —C(O)O—, —OC(O)—, —OC(O)N(R^{a})—, —N(R^{a})C(O)O—, —C(O)N(R⁵)O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R′, —NR⁵S(O)₂NR⁵R₆, —NR⁵S(O)₂R^{p}, —S(O)NR⁵R⁶, —S(O)R⁵, —NR⁵S(O)NR⁵R⁶, —NR⁵S(O)R^{p}, —C(O)R⁵, or —CO₂R₅, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR—S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₇cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR—S(O)₂NR⁵R⁶, —NR⁵S(O)₂R⁶, —S(O)NR⁵R⁶, —S(O)R⁵, —NR⁵S(O)NR⁵R⁶, —NR—S(O)R⁶, heterocycle, aryl, or heteroaryl; and wherein the heterocyclyl or heteroaryl is not attached via a nitrogen atom;
   R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R′, —NR⁵S(O)NR⁵R⁶, —NR⁵S(O)R⁶, heterocycle, aryl, or heteroaryl;
R³ is independently —H, —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;
R⁴ is independently —H, -D, —C₁-C₆alkyl, NH—NHR⁵, —NH—OR⁵, —O—NR⁵R⁶, —NHR⁵, —OR_{, —NHC(O)R}⁵, —NHC(O)NHR⁵, —NHS(O)₂R⁵, —NHS(O)₂NHR⁵, —S(O)₂OH, —C(O)OR⁵, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or
   R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CV) immediately below, or a pharmaceutically acceptable salt or solvate thereof:

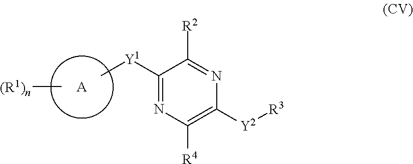

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S— or a direct bond;
Y² is —NR^{a}—, —(CR^{a}₂)ₘ—, —C(O)—, —C(R^{a})₂NH— —(CR^{a}₂)ₘO—, —C(O)N(R^{a})—, —N(R¹)C(O)—, —S(O)₂N(R^{a})—, —N(R^{a})S(O)₂, —N(R^{a})C(O)N(R^{a})—, N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁶S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)NR⁵R⁶, —NR⁵S(O)₂R⁶, —S(O)NR⁵R⁶, —S(O)R′, —NR{S(O)NR⁵R⁶, —NR⁵S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁶, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, —H, -D, —OH, —C₃-C₈cycloalkyl, or —C₁-C₆alkyl, wherein each alkyl or cycloalkyl is optionally substituted with one or more —NH, wherein 2 R^{a}, together with the carbon atom to which they are both attached, can combine to form a 3- to 8-membered cycloalkyl;
   R^{b} is independently, at each occurrence, —H, -D. —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR¹S(O)NR⁵R⁶, —NR⁵S(O)R⁶, heterocycle, aryl, heteroaryl, —(CH₂)ₙOH, —C₁-C₆alkyl, —CF₃, —CHF₂, or —CH₂F;
R³ is independently —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, —NH, heteroaryl, heterocyclyl, —(CH₂)ₙNH₂, —COOR^{b}, —CONHR^{b}, —CONH(CH₂)ₙCOOR^{b}, —NHCOOR^{b}, —CFs, —CHF₂, or —CH₂F;
R⁴ is independently —H, -D, —C₁-C₆alkyl, NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂—, —OH, —CN, —C(O)NR⁵R⁶, —S(O)₂NR⁵R⁶, C₃-C₆cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂—, or halogen; or R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂, in the heterocycle;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CW) immediately below, or a pharmaceutically acceptable salt or solvate thereof:

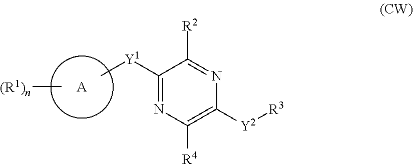

and pharmaceutically acceptable salts, prodrugs, solvates, hydrates, tautomers, or isomers thereof, wherein:
A is a 5- to 12-membered monocyclic or polycyclic cycloalkyl, heterocycloalkyl, aryl, or heteroaryl;
Y¹ is —S—, a direct bond, —NH—, —S(O)₂—, —S(O)₂—NH—, —C(═CH₂)—, —CH—, or —S(O)—;
Y² is —NR^{a}—, —(CR¾)m-, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂, —N(R^{a})C(O)N(R^{a})—, —N(R^{a})C(S)N(R^{a})—, —OC(O)N(R^{a})—, —N(R⁵)C(O)O—, —C(O)N(R^{a})O—, —N(R^{a})C(S)—, or —C(S)N(R⁵)—; wherein the bond on the left side of Y², as drawn, is bound to the pyrazine ring and the bond on the right side of the Y² moiety, as drawn, is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁶, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂RV, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —NH₂, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR—S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁶, —NR⁵S(O)NR⁵R⁶, —NR⁵S(O)R⁶, heterocycle, aryl, heteroaryl, —(CH₂)ₙOH, —C₁-C₆alkyl, —CF₃, —CHF₂, or —CH₂F;
R³ is independently —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or
R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, —NH₂, heteroaryl, heterocyclyl, —(CH₂)ₙNH₂, —COOR^{b}, —CONHR^{b}, —CONH(CH₂)ₙCOOR^{b}, —NHCOOR^{b}, —CF₃, —CHF₂, or —CH₂F;
R⁴ is independently —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen;
   R^{a} and R⁴, together with the atom or atoms to which they are attached, are combined to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently, at each occurrence, 1, 2, 3, 4, 5 or 6; and
n is independently, at each occurrence, 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CX) immediately below, or a pharmaceutically acceptable salt or solvate thereof:

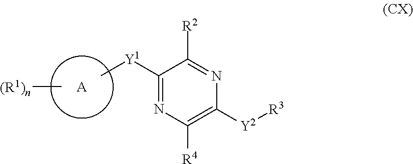

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
Y¹ is —S— or a direct bond;
Y² is selected from the group consisting of: —NR^{a}—, —(CR^{a}₂)ₘ—, —C(O)—, —C(R^{a})₂NH— (CR^{a}₂)ₘO—, —C(O)N(R′)—, —N(R^{a})C(O)—, —S(O)₂N(R^{a})—, —N(R^{a})S(O)₂, —N(R¹)C (O)N(R^{a})—, —N(R^{a})C (S)N(R′)—, —C(O)O—, —OC (O)—, —OC(O)N(R^{a})—, —N(R^{a})C(O)O—, —C(O)N(R^{a})O—N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;
R¹ is independently, at each occurrence, —H, -D, C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R₅, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, selected from the group consisting of —H, -D, —OH, —C₃-C₆cycloalkyl, and —C₁-C₆alkyl, wherein each alkyl or cycloalkyl is optionally substituted with one or more —NH₂—, wherein 2 R^{a}, together with the carbon atom to which they are both attached, can combine to form a 3- to 8-membered cycloalkyl;
   R^{b} is independently —H, -D, —C₁-C₆alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂—; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;
R⁴ is independently, at each occurrence, —H, -D, or —C₁-C₆alkyl, wherein each alkyl is optionally substituted with one or more —OH, —NH₂—, halogen, or oxo; or
   R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR, halogen, —NR⁷R⁸, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CY) immediately below, or a pharmaceutically acceptable salt or solvate thereof:

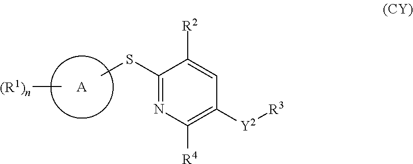

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
Y² is selected from the group consisting of: —NR^{a}—, —(CR^{a}₂)ₘ—, —C(O)—, —C(R^{a})₂NH— (CR^{a}₂)ₘO—, —C(O)N(R^{a})—, —N(R)C(O)—, —S(O)₂N(R^{a})—, —N(R^{a})S(O)₂, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₆cycloalkyl, —OH, halogen, —NO₂, —CN, —NR⁵R⁶, —SR′, —S(O)₂NR⁵R⁶, —S(O)₂R, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR^{b}, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, selected from the group consisting of—H, -D, —OH, —C₃-C₆cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₁-C₆alkyl, —C₃-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR_{, —NR}⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;
R⁴ is independently, at each occurrence, —H, -D, or —C₁-C₆alkyl, wherein each alkyl is optionally substituted with one or more —OH, —NH₂, halogen, or oxo; or
   R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (CZ) immediately below, or a pharmaceutically acceptable salt or solvate thereof:

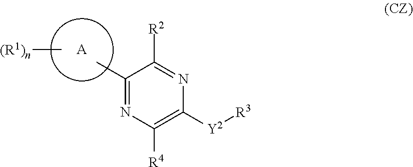

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
Y² is selected from the group consisting of: —NR^{a}—, —(CR^{a}₂)ₘ—, —C(O)—, —C(R^{a})₂NH— (CR^{a}₂)ₘO—, —C(O)N(R′)—, —N(R^{a})C(O)—, —S(O)₂N(R^{a})—, —N(R^{a})S(O)₂, —N(R^{a})C(O)N(R^{a})—, —N(R^{a})C(S)N(R′)—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R;
R¹ is independently, at each occurrence, —H, -D, —C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R, or —CO₂R₅, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶. —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂—, wherein 2 R^{a}, together with the carbon atom to which they are both attached, can combine to form a 3- to 8-membered cycloalkyl;
   R^{b} is independently —H, -D, —C₁-C₆alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;
R⁴ is independently, at each occurrence, —H, -D, or —C₁-C₆alkyl, wherein each alkyl is optionally substituted with one or more —OH, —NH₂, halogen, or oxo; or
   R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₆cycloalkyl, a monocyclic or polycyclic 3- to 12-membered heterocycle, —OR⁷, —SR⁷, halogen, —NR⁷, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (DA) immediately below, or a pharmaceutically acceptable salt or solvate thereof:

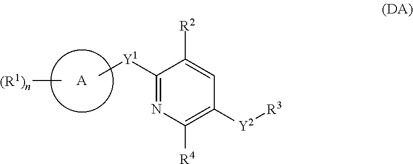

or a pharmaceutically acceptable salt, prodrug, solvate, hydrate, tautomer, or isomer thereof, wherein:
A is selected from the group consisting of 5- to 12-membered monocyclic or polycyclic cycloalkyl, heterocycloalkyl, aryl, or heteroaryl;
Y¹ is —S— or a direct bond;
Y² is selected from the group consisting of: —NR^{a}—, —(CR^{a}₂)ₘ—, —C(O)—, —C(R^{a})₂NH—(CR^{a}₂)ₘO—, —C(O)N(R^{a})—, —N(R^{a})C(O)— —S(O)₂N(R^{a})—, —N(R^{a})S(O)₂—, —N(R^{a})C(O)N(R^{a})—, —N(R^{a})C(S)N(R^{a})—, —C(O)O—, —OC(O)— —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;
R¹ is independently, at each occurrence, —H, -D, —C₁-C₆alkyl, —C₂—C alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR—S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R, —OR^{b}, —NR⁵R⁶, —SW, —S(O)₂NR⁵R⁶, —S(O)₂W, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₁-C₆alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN. —R⁵, —OR⁵, —NR⁵R⁶, —SR⁵, —S(O)₂NR⁵R⁶, —S(O)₂R⁵. —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;
R³ is independently, at each occurrence, selected from the group consisting of —H, C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂—, —OR^{a}, —NHR^{a}, —(CH₂)ₙOH, heterocyclyl, or spiroheterocycl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂—, heteroaryl, heterocyclyl, —(CH₂)ₙNH₂, —COOR^{a}, —CONHR^{b}, —CONH(CH₂)ₙCOOR^{a}, —NHCOOR³, —CF₃, C—F₂, or CH₂F;
R⁴ is independently, at each occurrence, —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₆cycloalkyl, a monocyclic or polycyclic 3- to 12-membered heterocycle, —OR⁷, —SR⁷, halogen, —NR⁷, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (DB) immediately below, or a pharmaceutically acceptable salt or solvate thereof:

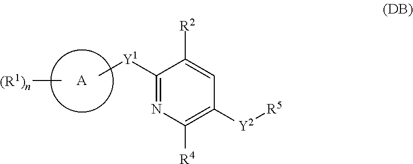

or a pharmaceutically acceptable salt, prodrug, solvate, hydrate, tautomer, or isomer thereof, wherein:
A is selected from the group consisting of 5- to 12-membered monocyclic or polycyclic cycloalkyl, heterocycloalkyl, aryl, or heteroaryl;
Y¹ is —S—, a direct bond, —NH—, —S(O)₂—, —S(O)₂—NH—, —C(═CH₂)—, —CH—, or —S(O)—;
Y² is selected from the group consisting of: —NR^{a}—, —(CR^{a}₂)ₘ—, —C(O)—, —C(R^{a})₂NH—(CR^{a}₂)ₘO—, —C(O)N(R^{a})—, —N(R^{a})C(O)—, —S(O)₂N(R^{a})—, —N(R^{a})S(O)₂, —N(R^{a})C (O)N(R^{a})—, —N(R^{a})C (S)N(R′)—, —C(O)O—, —OC(O)—, —OC_{(O)N(R}^{a})—, —N(R^{a})C(O)O—, —C(O)N(R^{a})O—N(R^{a})C(S)—, —C(S)N(R^{a})—, and —OC(O)O—; wherein the bond on the left side of Y2, as drawn, is bound to the pyridine ring and the bond on the right side of the Y² moiety, as drawn, is bound to R³;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R₅, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
R² is independently —OR^{b}, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, aryl, heterocyclyl containing 1-5 heteroatoms selected from the group consisting of N, S. P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵. —NR⁵R⁶, —SR⁵, —S(O)₂NR⁵R⁶. —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
   R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₁-C₆alkyl, —C₃-C_{R}cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R₅, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CH—F₂, or CH₂F;
R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;
R⁴ is independently, at each occurrence, —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂OH, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or
   R^{a} and R⁴, together with the atom or atoms to which they are attached, can combine to form a monocyclic or polycyclic C₃—C₁₋₂cycloalkyl, or a monocyclic or polycyclic 3- to 12-membered heterocycle, wherein the cycloalkyl or heterocycle is optionally substituted with oxo; wherein the heterocycle optionally comprises —S(O)₂, in the heterocycle;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —ORC, —SR⁷, halogen, —NR⁷R⁸, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (DC) immediately below, or a pharmaceutically acceptable salt or solvate thereof:

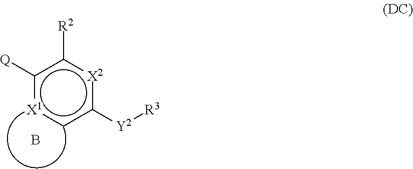

and pharmaceutically acceptable salts, prodrugs, solvates, hydrates, tautomers, or isomers thereof, wherein:
Q is H or

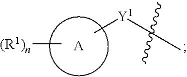

A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁵, —C(O)R⁵, or —CO₂R₅, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
Y¹ is —S—, a direct bond, —NH—, —S(O)₂—, —S(O)₂—NH—, —C(═CH₂)—, —CH—, or —S(O)—;
X¹ is N or C
X² is N or CH;
B, including the atoms at the points of attachment, is a monocyclic or polycyclic 5- to 12-membered heterocycle or a monocyclic or polycyclic 5- to 12-membered heteroaryl;
R² is independently H—, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH— (CR^{a}₂)ₙO—, —C(O)N(R^{a})—, —N(R^{a})C(O)—, —S(O)₂N(R^{a})—, —N(R^{a})S(O)₂, —N(R^{a})C(O)N(R^{a})—, —N(R^{a})C(S)N(R^{a})—, —C(O)O—. —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
   R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₁-C₆alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR—S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;
R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)nCOOR^{a}, —NHCOOR³, —CF₃, CHF₂, or CH₂F;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;
   R⁷ and R₈ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (DD) immediately below, or a pharmaceutically acceptable salt or solvate thereof:

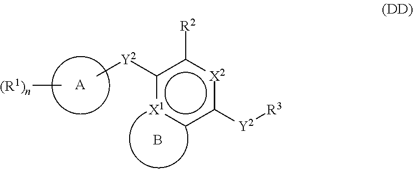

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁶, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵ S(O)NR⁵R⁶, —NR⁵S(O)R⁶, heterocycle, aryl, or heteroaryl;
Y¹ is —S—, a direct bond, —NH—, —S(O)₂—, —S(O)₂—NH—, —C(═CH₂)—, —CH—, or —S(O)—;
X¹ is N or C
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR, —NR⁵R⁶, —SR⁶, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
   R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₁-C₆alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R′, —NR⁵S(O)NR⁵R⁶, —NR_{S(O)R}⁶, heterocycle, aryl, heteroaryl, —(CH₂)ₙOH, —C₁-C₆alkyl, CF₃, CH—F₂, or CH₂F;
R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (DE) immediately below, or a pharmaceutically acceptable salt or solvate thereof:

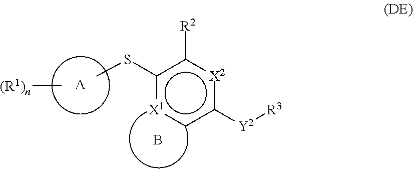

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —C₂R₅, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁶, —NR⁵S(O)NR⁵R⁶, —NR⁵S(O)R⁶, heterocycle, aryl, or heteroaryl;
X¹ is N or C;
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R¹)—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;
   R^{a} is independently, at each occurrence, selected from the group consisting of —H, -D, —OH, —C₃-C₈cycloalkyl, and —C₃-C₈alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₃-C₈alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR, —NR⁵R⁶, —SR⁵, —S(O)₂NR⁵R⁶, —S(O)₂R⁵, —NR⁶S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;
R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CH—F₂, or CH₂F;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor has the formula (DF) immediately below, or a pharmaceutically acceptable salt or solvate thereof:

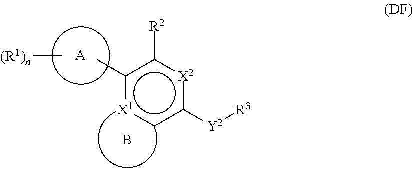

and pharmaceutically acceptable salts, prodrugs, solvates, hydrates, tautomers, or isomers thereof, wherein:
A is selected from the group consisting of 5- to 12-membered monocyclic or polycyclic cycloalkyl, heterocycloalkyl, aryl, or heteroaryl;
R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)_{2N}R₅R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;
X¹ is N or C:
X² is N or CH;
B, including the atoms at the points of attachment, is a monocyclic or polycyclic 5- to 12-membered heterocycle or a monocyclic or polycyclic 5- to 12-membered heteroaryl;
R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;
Y² is selected from the group consisting of: —NR^{a}—, —(CR^{a}₂)ₘ—, —C(O)—, —C(R^{a})₂NH—(CR^{a}₂)ₘO—, —C(O)N(R^{a})—, —N(R^{a})C(O)—, —S(O)₂N(R^{a})—, —N(R^{a})S(O)₂, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—. —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³.
   R^{a} is independently, at each occurrence, selected from the group consisting of —H, -D, —OH, —C₃-C₆cycloalkyl, and C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;
   R^{b} is independently —H, -D, —C₁-C₆alkyl, —C₃-C₈cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;
R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or
R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR³, —CF₃, CHF₂, or CH₂F;
R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;
   R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;
m is independently 1, 2, 3, 4, 5 or 6; and
n is independently 0, 1, 2, 3, 4, 5, 6, 7, 8, 9, or 10;
as used in the present embodiment, the term “cycloalkyl” means monocyclic or polycyclic saturated carbon rings containing 3-18 carbon atoms. As used in the present embodiment, the term “cycloalkenyl” means monocyclic, non-aromatic unsaturated carbon rings containing 4-18 carbon atoms. As used in the present embodiment, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized p electrons (aromaticity) shared among the ring carbon or heteroatoms. As used in the present embodiment, “heterocyclyl” or“heterocycloalkyl” or“heterocycle” as used in this paragraph may mean a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH2- group can optionally be replaced by a —C(O)— or a ring sulfur atom may be optionally oxidised to form the S-oxides. As used in the present embodiment, “Spirocycle” or“spirocyclic” means carbogenic bicyclic ring systems with both rings connected through a single atom, one or both of the rings in a spirocycle can be fused to another carbocyclic, heterocyclic, aromatic, or heteroaromatic ring, one or more of the carbon atoms in the spirocycle can be substituted with a heteroatom (e.g., O, N, S, or P). As used in the present embodiment, the term “spirocyclic heterocycle,” “spiroheterocyclyl,” or“spiroheterocycle” is understood to mean a spirocycle wherein at least one of the rings is a heterocycle. As used in the present embodiment, the term “aryl” refers to cyclic, aromatic hydrocarbon groups that have 1 to 2 aromatic rings, including monocyclic or bicyclic groups. As used in the present embodiment, unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C.

In embodiments, the SHP2 inhibitor is a compound of having the formula (DG) immediately below, or a pharmaceutically acceptable salt or solvate thereof:

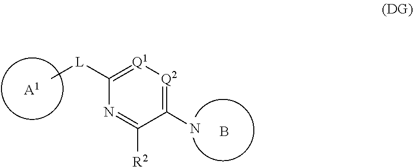

wherein:
ring A¹ is a fused tricyclic heteroaryl or cycloalkyl fused bicyclic heteroaryl ring substituted with R^{a}, R^{b}, and/or R^{c}
wherein R^{a} and R^{b} are independently selected from hydrogen, alkyl, amino, cycloalkyl, alkylidenyl, alkenyl, halo, haloalkyl, alkoxy, haloalkoxy, hydroxy, hydroxyalkyl, alkoxyalkyl, cyano, aminoalkyl, carboxy, and alkoxycarbonyl and
R^{c} is hydrogen, alkyl, halo, hydroxy, alkoxy, optionally substituted heterocyclyl, optionally substituted aryl, optionally substituted heteroaryl, —S(O)R, S(O)₂R, —C(O)R, —OR′, —NR′C(O)R, —NR′SO₂R, —OC(O)NR′R″, —C(O)NR′R″, —S(O)₂NR′R″, —NR′R″, or —NR′C(O)C(O)R
   where R is alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl, and
   R′ and R″ are independently hydrogen, alkyl, hydroxyalkyl, alkoxyalkyl, aminoalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; or R′ and R″ together with the nitrogen atom to which they are attached form optionally substituted heterocyclyl; or
when R^{c} and R^{a} are attached to the same carbon of fused heteroaryl ring, then R^{c} and R^{a} together with the carbon atom to which they are attached can form cycloalkylene or heterocyclylene;
Q¹ is N or CR¹ wherein R¹ is hydrogen or deuterium;
Q² is N or CH, or CD;
R² is alkyl, halo, hydroxy, hydroxyalkyl, —CD₂OH, alkylsulfoxide, alkylsulfonyl, aminosulfonyl, aminocarbonyl, carboxy, cyano, or alkoxycarbonyl;
L is bond, O, S, S(O), S(O)₂, or CR⁵R⁶ where R⁵ and R⁶ are independently hydrogen or alkyl;
and

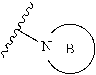

is a ring of formula (a) or (b)

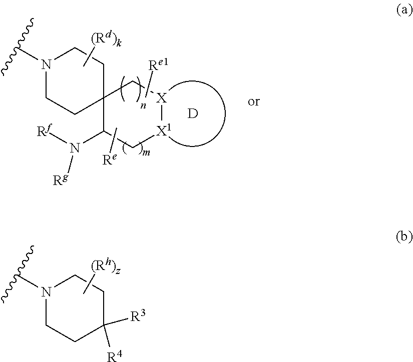

wherein:
   m is 0, 1, or 2;
   n is 0, 1, or 2 wherein when n is 2 then one of the CH₂ can be replaced with 0, S, or SO₂; provided m+n is 1, 2, or 3;
   k is 0, 1 or 2
   z is 0, 1, or 2
   each R^{d} is independently hydrogen, alkyl, or halogen; R^{e} and R^{e1} are independently hydrogen, alkyl, halogen, haloalkyl, alkoxy, haloalkoxy, hydroxy, hydroxyalkyl, cyano or oxo; or
   when R^{e} and R^{e1} are attached to the same carbon atom, then R^{e} and R^{e1} together with the carbon atom to which they are attached can form cycloalkylene or heterocyclylene;
   R^{f} and R^{s} are independently hydrogen, alkyl, or haloalkyl;
   each R^{h} is independently alkyl, halo, haloalkyl, alkoxy, hydroxyalkyl, alkoxyalkyl, hydroxy, cyano, or oxo; or
   when one R^{h} is connected to carbon 2 or 3 of the piperidine (b) ring and the second R^{h} is attached to carbon 5 or 6 of the piperidine (b) ring, the nitrogen atom being position 1, then the first and second R^{h} can combine to form alkylene chain;
   ring D is phenyl or a 5 or 6 membered heteroaryl ring which, including X and X¹, contains one to three heteroatoms independently selected from N, O, and S and ring D can optionally be substituted with one or two groups independently selected from alkyl, halo, haloalkyl, alkoxy, haloalkoxy, hydroxy, hydroxyalkyl, cyano, amino, aminoalkyl, carboxy, cycloalkyl, heterocyclyl, heteroaryl, and acylamino;
   X and X¹ are independently N or C; provided that only one of X and X¹ can be N;
R³ is amino or aminoalkyl;
R⁴ is alkyl, cycloalkylalkyl, halo, hydroxy, amino, alkoxycarbonyl, hydroxyalkyl, alkoxyalkyl, arylalkyl, heterocyclealkyl, cycloalkylalkyl, heterocyclealkyl, 5 or 6 membered heteroaryl, or 4 to 6 membered heterocyclyl wherein heteroaryl by itself or as part of heteroaralkyl and heterocyclyl by itself or as part of heterocyclylalkyl are substituted with R¹ and/or R¹ independently selected from hydrogen, alkyl, halo, haloalkyl, haloalkoxy, alkoxy, hydroxy, cyano, amino, aminoalkyl, alkylsulfoxide, and alkyl sulfonyl; or
   R³ and R⁴ together with the carbon atom to which they are attached form a ring of formula (c):

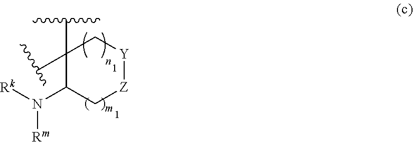

wherein:
m1 is 0, 1, or 2;
n1 is 0, 1, or 2; provided m1+n1 is 1, 2, or 3;
R^{k} and R^{m} are independently hydrogen, alkyl, or haloalkyl; one of Y and Z is CH₂, O, S, S(O), S(O)₂, or NH; and the other of X and Y is CH₂; and wherein ring of formula (c) is substituted with Rⁿ and/or R^{o} independently selected from hydrogen, alkyl, alkylidenyl, alkenyl, halo, haloalkyl, haloalkoxy, alkoxy, hydroxy, cyano, alkylsulfoxide, alkylsulfonyl, oxo, cycloalkyl, heterocyclyl, and heteroaryl; or
when Rⁿ and R^{o} are attached to the same carbon atom, then Rⁿ and R^{o} together with the carbon atom to which they are attached can form cycloalkylene or heterocyclylene; or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive.

In embodiments, the SHP2 inhibitor is a compound of having the formula (DH) immediately below, or a pharmaceutically acceptable salt or solvate thereof:

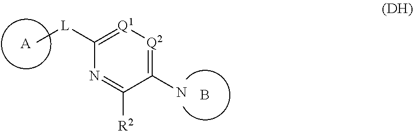

wherein:
ring A is aryl, cycloalkyl, heteroaryl, or fused heteroaryl ring, each ring substituted with R^{a}, R^{b}, and/or R^{c};
   wherein R^{a} and R^{b} are independently selected from hydrogen, alkyl, amino, cycloalkyl, alkylidenyl, alkenyl, halo, haloalkyl, alkoxy, haloalkoxy, hydroxy, hydroxyalkyl, alkoxyalkyl, cyano, aminoalkyl, carboxy, and alkoxycarbonyl; and
   R^{c} is hydrogen, alkyl, halo, hydroxy, alkoxy, optionally substituted heterocyclyl, optionally substituted aryl, optionally substituted heteroaryl, —S(O)R, S(O)₂R, —C(O)R, —OR′, —NR′C(O)R, —NR′SO₂R, —OC(O)NR′R″, —C(O)NR′R″, —S(O)₂NR′R″, —NR′R″, or —NR′C(O)C(O)R;
      where R is alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl, and
      R′ and R″ are independently hydrogen, alkyl, hydroxyalkyl, alkoxyalkyl, aminoalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; or R′ and R″ together with the nitrogen atom to which they are attached form optionally substituted heterocyclyl; or
   when R^{c} and R^{a} are attached to the same carbon of cycloalkyl or fused heteroaryl ring, then R^{c} and R^{a} together with the carbon atom to which they are attached can form cycloalkylene or heterocyclylene;
Q¹ is N or CR¹ wherein R¹ is hydrogen or deuterium;
Q² is N or CH, or CD; R² is alkyl, halo, hydroxy, hydroxyalkyl, —CD₂OH, alkylsulfoxide, alkylsulfonyl, aminosulfonyl, aminocarbonyl, carboxy, cyano, or alkoxycarbonyl;
L is bond, O, S, S(O), S(O)₂, or CR⁵R⁶ where R⁵ and R⁶ are independently hydrogen or alkyl;
and

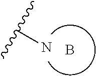

is a ring of formula (a) or (b):

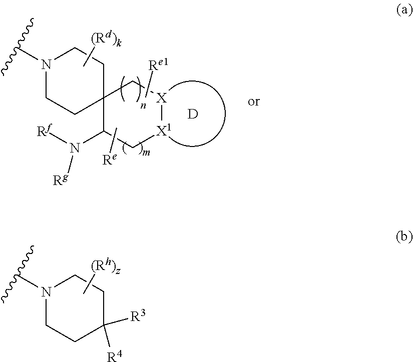

wherein:
m is 0, 1, or 2;
n is 0, 1, or 2 wherein when n is 2 then one of the CH₂ can be replaced with 0, S, or SO₂; provided m+n is 1, 2, or 3;
k is 0, 1 or 2;
z is 0, 1, or 2;
each R^{d} is independently hydrogen, alkyl, or halogen;
R^{e} and R^{e1} are independently hydrogen, alkyl, halogen, haloalkyl, alkoxy, haloalkoxy, hydroxy, hydroxyalkyl, cyano or oxo; or
when R^{e} and R^{e1} are attached to the same carbon atom, then R^{e} and R^{e1} together with the carbon atom to which they are attached can form cycloalkylene or heterocyclylene;
R^{f} and R^{s} are independently hydrogen, alkyl, or haloalkyl;
each R^{h} is independently alkyl, halo, haloalkyl, alkoxy, hydroxyalkyl, alkoxyalkyl, hydroxy, cyano, or oxo; or
when one R^{h} is connected to carbon 2 or 3 of the piperidine (b) ring and the second R^{h} is attached to carbon 5 or 6 of the piperidine (b) ring, the nitrogen atom being position 1, then the first and second R^{h} can combine to form alkylene chain;
ring D is phenyl or a 5 or 6 membered heteroaryl ring which, including X and X¹, contains one to three heteroatoms independently selected from N, O, and S and ring D can optionally be substituted with one or two groups independently selected from alkyl, halo, haloalkyl, alkoxy, haloalkoxy, hydroxy, hydroxyalkyl, cyano, amino, aminoalkyl, carboxy, cycloalkyl, heterocyclyl, heteroaryl, and acylamino;
X and X¹ are independently N or C; provided that only one of X and X¹ can be N; R³ is amino or aminoalkyl;
R⁴ is alkyl, cycloalkylalkyl, halo, hydroxy, amino, alkoxycarbonyl, hydroxyalkyl, alkoxyalkyl, arylalkyl, heterocyclealkyl, cycloalkylalkyl, heterocyclealkyl, 5 or 6 membered heteroaryl, or 4 to 6 membered heterocyclyl wherein heteroaryl by itself or as part of heteroaralkyl or heterocyclyl by itself or as part of heterocyclyl alkyl is substituted with R¹ and/or R¹ independently selected from hydrogen, alkyl, halo, haloalkyl, haloalkoxy, alkoxy, hydroxy, cyano, amino, aminoalkyl, alkylsulfoxide, and alkylsulfonyl; or
R³ and R⁴ together with the carbon atom to which they are attached form a ring of formula (c):

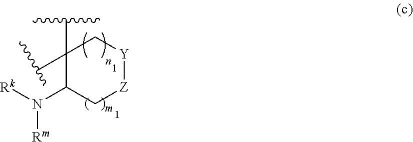

wherein:
m1 is 0, 1; or 2;
n1 is 0, 1, or 2; provided m1+n1 is 1, 2, or 3;
R^{k} and R^{m} are independently hydrogen, alkyl, or haloalkyl;
one of Y and Z is CH₂, O, S, S(O), S(O)₂, or NH; and the other of X and Y is CH₂; and wherein ring of formula (c) is substituted with Rⁿ and/or R^{o} independently selected from hydrogen, alkyl, alkylidenyl, alkenyl, halo, haloalkyl, haloalkoxy, alkoxy, hydroxy, cyano, alkylsulfoxide, alkylsulfonyl, oxo, cycloalkyl, heterocyclyl, and heteroaryl; or
when Rⁿ and R^{o} are attached to the same carbon atom, then Rⁿ and R^{o} together with the carbon atom to which they are attached can form cycloalkylene or heterocyclylene; or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive.

In embodiments, the SHP2 inhibitor is a compound of having the formula (DI) immediately below, or a pharmaceutically acceptable salt or solvate thereof:

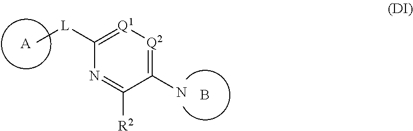

wherein:
ring A is aryl, cycloalkyl, heteroaryl, or fused heteroaryl ring, each ring substituted with R^{a}, R^{b}, and/or R^{c};
   wherein R^{a} and R^{b} are independently selected from hydrogen, alkyl, amino, cycloalkyl, alkylidenyl, alkenyl, halo, haloalkyl, alkoxy, haloalkoxy, hydroxy, hydroxyalkyl, cyano, aminoalkyl, carboxy, and alkoxycarbonyl; and
   R^{c} is hydrogen, alkyl, halo, hydroxy, alkoxy, optionally substituted heterocyclyl, optionally substituted aryl, optionally substituted heteroaryl, —NHCOR, or —NR′R″;
      where R is alkyl, cycloalkyl, optionally substituted heterocyclyl, optionally substituted aryl, optionally substituted heteroaryl, or optionally substituted heteroaralkyl, and
      R′ and R″ are independently hydrogen or alkyl; or R′ and R″ together with the nitrogen atom to which they are attached form optionally substituted heterocyclyl; or
   when R^{c} and R^{a} are attached to the same carbon of cycloalkyl or fused heteroaryl ring, then R^{c} and R^{a} together with the carbon atom to which they are attached can form cycloalkylene or heterocyclylene;
Q¹ is N or CR¹ wherein R¹ is hydrogen or deuterium;
Q² is N, CH, or CD;
R² is alkyl, halo, hydroxy, hydroxyalkyl, —CD₂OH, alkylsulfoxide, alkylsulfonyl, aminosulfonyl, aminocarbonyl, carboxy, cyano, or alkoxycarbonyl;
L is bond, O, S, SO, SO₂, or CR⁵R⁶ where R⁵ and R⁶ are independently hydrogen or alkyl;
and

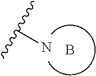

is a ring of formula (a) or (b):

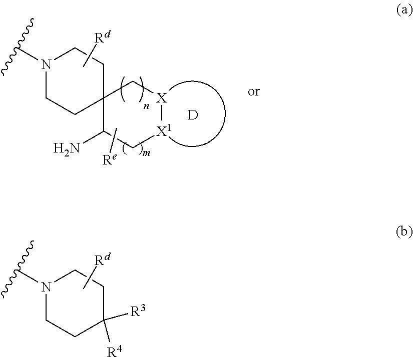

wherein:
m is 0, 1; or 2;
n is 0, 1, or 2; provided m+n is 1, 2, or 3;
R^{d} is hydrogen or alkyl;
R^{e} is hydrogen, alkyl, halogen, or oxo;
R^{h} is independently alkyl, halo, haloalkyl, alkoxy, hydroxyalkyl, alkoxyalkyl, hydroxy, cyano, or oxo;
ring D is phenyl or a 5 or 6 membered heteroaryl ring which, including X and X, contains one to three heteroatoms independently selected from N, O, or S and ring D can optionally be substituted with alkyl;
X and X¹ are independently N or C; provided that only one of X and X¹ can be N;
R³ is amino or aminoalkyl; R⁴ is alkyl, cycloalkylalkyl, halo, hydroxy, amino, alkoxycarbonyl, hydroxyalkyl, alkoxyalkyl, 5 or 6 membered heteroaryl, or 4 to 6 membered heterocyclyl wherein heteroaryl or heterocyclyl is substituted with R¹ and/or R¹ independently selected from hydrogen, alkyl, halo, haloalkyl, haloalkoxy, alkoxy, hydroxy, cyano, alkylsulfoxide, and alkylsulfonyl; or
R³ and R⁴ together with the carbon atom to which they are attached form a ring of formula (C):

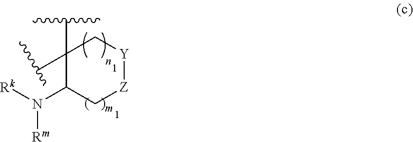

wherein:
m1 is 0, 1; or 2;
n1 is 0, 1, or 2; provided m1+n1 is 1, 2, or 3;
one of Y and Z is CH₂, O, S, SO, SO₂, or NH; and the other of X and Y is CH₂; and wherein ring of formula (C) is substituted with Rⁿ and/or R^{o} independently selected from hydrogen, alkyl, alkylidenyl, alkenyl, halo, haloalkyl, haloalkoxy, alkoxy, hydroxy, cyano, alkylsulfoxide, alkylsulfonyl, and oxo; or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive.

In embodiments, the SHP2 inhibitor is a compound of having the formula (DJ) immediately below, or a pharmaceutically acceptable salt or solvate thereof:

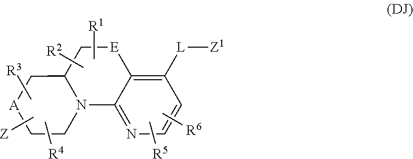

wherein:
A and E are independently selected from a bond, CH₂, O, NH, S, and S(O)₂;
Z is hydrogen, alkyl, halo, haloalkyl, haloalkoxy, cyano, cycloalkyl, heterocyclyl, heteroaryl (wherein cycloalkyl, heterocyclyl, and heteroaryl are optionally substituted with one to three halo), —O(alk)_{y}R^{a}, —O(alk)OR^{b}, —S(O)R^{c}, —S(O)₂R^{d}, —NR^{e}C(O)R^{f}, —NR^{s}SO₂R^{b}, —OC(O)NRⁱR^{j}, —C(O)NR^{k}R^{m}, —S(O)₂NRⁿR^{o}, —NR^{p}R^{q}, —NR^{r}C(O)C(O)R^{s} or —Y-M (wherein Y is bond, O, or SO₂ and M is alkyl, haloalkyl, cycloalkyl, heterocyclyl, or heteroaryl wherein alkyl, haloalkyl, cycloalkyl, heterocyclyl and heteroaryl are substituted with —O(alk)_{y}R^{a}, —O(alk)OR^{b}, —S(O)R^{c}, —S(O)₂R^{d}, —NR^{e}C(O)R^{f}, —NR^{s}SO₂R^{h}, —OC(O)NRⁱR^{j}, —C(O)NR^{k}R^{m}, —S(O)₂NRⁿR^{o}, —NR^{p}R^{q}, or —NR^{r}C(O)C(O)R^{s} and cycloalkyl, heterocyclyl, and heteroaryl are optionally further substituted with 1 to 3 halo);
wherein each y is 0 or 1,
each alk is alkylene, and
   each R^{c}, R^{d}, R^{f}, R^{h}, and R^{s} are independently alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; and
   each R^{a}, R^{b}, R^{e}, R^{g}, Rⁱ, R^{j}, R^{k}, R^{m}, Rⁿ, R^{o}, R^{p}, R^{q}, R^{r}, and R^{s} are independently hydrogen, alkyl, cycloalkyl, cycloalkylalkyl, hydroxyalkyl, alkoxyalkyl, aminoalkyl, optionally substituted heterocyclyl, optionally substituted heterocyclylalkyl, optionally substituted aryl, optionally substituted aralkyl, optionally substituted heteroaryl, or optionally substituted heteroaralkyl; or, independently of each other,
   each Rⁱ and R^{j}, R^{k} and R^{m}, Rⁿ and R^{o}, and R^{p} and R^{q}, together with the nitrogen atom to which they are attached form optionally substituted heterocyclyl;
R¹, R², R³, and R⁴ are independently selected from hydrogen, alkyl, cycloalkyl, halo, haloalkyl, haloalkoxy, alkoxy, cyano, hydroxy, hydroxylalkyl, amino, and aminoalkyl;
or one of R¹ and R², and R³ and R⁴, when attached to the same carbon, combine to form oxo, alkylidenyl, 3 to 6 membered cycloalkylene, or 4 to 6 membered optionally substituted heterocyclylene;
R⁵ and R⁶ are independently selected from hydrogen, alkyl, cycloalkyl, halo, haloalkyl, haloalkoxy, alkoxy, hydroxy, cyano, hydroxylalkyl, amino, and aminoalkyl, or one of R⁵ and R⁶ is optionally substituted heterocyclyl and the other of R⁵ and R⁶ is selected from hydrogen, alkyl, cycloalkyl, halo, haloalkyl, haloalkoxy, alkoxy, hydroxy, cyano, hydroxylalkyl, amino, and aminoalkyl;
L is bond, O, S, S(O), S(O)₂, or CR⁷R⁸ where R⁷ and R⁸ are independently hydrogen or alkyl;
Z¹ is a group of formula (a) or (b):

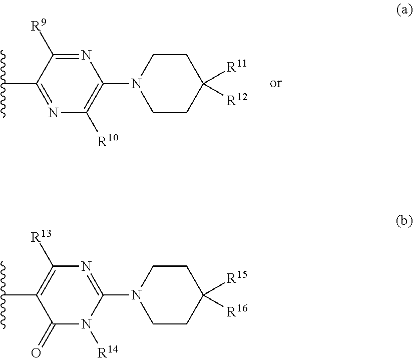

wherein:
   R⁹ is hydrogen, alkyl, halo, hydroxy, amino, or haloalkyl; R¹⁰ is hydrogen, alkyl, halo, hydroxy, hydroxyalkyl, —CD₂OH, alkylsulfoxide, alkylsulfonyl, amino, aminoalkyl, aminosulfonyl, aminocarbonyl, carboxy, cyano, or alkoxycarbonyl;
R¹³ is hydrogen, alkyl, halo, hydroxy, amino, or haloalkyl;
R¹⁴ is hydrogen, alkyl, or haloalkyl;
   R¹¹ and R¹⁵ are selected from amino and aminoalkyl;
R¹² and R¹⁶ are selected from hydrogen, cyano, halo, alkyl, haloalkyl, alkoxy, haloalkoxy, cycloalkyl, aryl, heterocyclyl, and heteroaryl, where alkyl, cycloalkyl, aryl, heterocyclyl and heteroaryl are optionally substituted with one to three substituents independently selected from alkyl, halo, haloalkyl, haloalkoxy, alkoxy, and cyano;
   or R¹¹ and R¹², and R¹⁵ and R¹⁶ together with the carbon atom to which they are attached form a ring of formula (c):

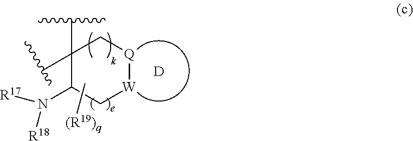

wherein:
e is 0, 1, or 2;
k is 0, 1, or 2 provided e+k is 1, 2, or 3;
q is 0, 1, or 2, or 3;
R¹⁷ and R¹⁸ are independently selected from hydrogen, alkyl, cycloalkyl, and haloalkyl;
each R¹⁹ is independently selected from hydrogen, alkyl, halo, haloalkyl, haloalkoxy, alkoxy, hydroxy, cyano, alkylsulfoxide, alkylsulfonyl, oxo, cycloalkyl, optionally substituted heterocyclyl, and optionally substituted heteroaryl; or
when two R¹⁹ groups are attached to the same carbon atom, the two R¹⁹ groups together with the carbon atom to which they are attached form cycloalkylene or heterocyclylene.
ring D is absent or present; wherein:
   (i) when ring D is absent, then one of Q and W is CH₂, O, S, S(O), S(O)₂, or NH; and the other of Q and W is CH₂; and
   (ii) when ring D is present, then Q and W are independently N or C provided only one of Q and W is N; and ring D is phenyl or a 5 or 6 membered heteroaryl ring which, including Q and W, contains one to three heteroatoms independently selected from N, O, and S and ring D is optionally substituted with one or two substituents independently selected from alkyl, cycloalkyl, halo, haloalkyl, alkoxy, haloalkoxy, hydroxy, hydroxyalkyl, cyano, amino, aminoalkyl, carboxy, and optionally substituted heterocyclyl; or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments, the SHP2 inhibitor is a compound of having the formula (DK) immediately below, or a pharmaceutically acceptable salt or solvate thereof:

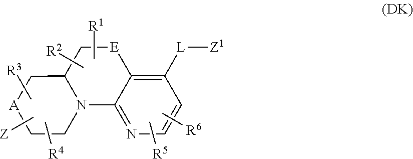

wherein:
A and E are independently selected from a bond, CH₂, O, NH, S, and S(O)₂;
Z is hydrogen, alkyl, halo, haloalkyl, haloalkoxy, cyano, cycloalkyl, heterocyclyl, heteroaryl (wherein cycloalkyl, heterocyclyl, and heteroaryl are optionally substituted with one to three halo),
   —O(alk)_{y}R^{a}, —O(alk)OR^{b}, —S(O)R^{c}, —S(O)₂R^{d}, —NR^{e}C(O)R, —NR^{s}SO₂R^{h}, —OC(O)NRⁱR^{j}, —C(O)NR^{k}R^{m}, —S(O)₂NRⁿR^{o}, —NR^{p}R^{q}, —NR^{r}C(O)C(O)R^{s} or
   —Y-M (wherein Y is bond, 0, or SO₂ and M is alkyl, haloalkyl, cycloalkyl, heterocyclyl, or heteroaryl
   wherein alkyl, haloalkyl, cycloalkyl, heterocyclyl and heteroaryl are substituted with —O(alk)_{y}R^{a}, —O(alk)OR^{b}, —S(O)R^{c}, —S(O)₂R^{d}, —NR^{e}C(O)R, —NR^{s}SO₂R^{h}, —OC(O)NRⁱR^{j}, —C(O)NR^{k}R^{m}, —S(O)₂NRⁿR^{o}, —NR^{p}R^{q}, or —NR^{r}C(O)C(O)R^{s} and
   cycloalkyl, heterocyclyl, and heteroaryl are optionally further substituted with 1 to 3 halo);
      wherein each y is 0 or 1,
      each alk is alkylene, and
      each R^{c}, R^{d}, R^{f}, R^{h}, and R^{s} are independently alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; and
      each R^{a}, R^{b}, R^{e}, R^{g}, Rⁱ, R^{j}, R^{k}, R^{m}, Rⁿ, R^{o}, R^{p}, R^{q}, R^{r}, and R^{s} are independently hydrogen, alkyl, cycloalkyl, cycloalkylalkyl, hydroxyalkyl, alkoxyalkyl, aminoalkyl, optionally substituted heterocyclyl, optionally substituted heterocyclylalkyl, optionally substituted aryl, optionally substituted aralkyl, optionally substituted heteroaryl, or optionally substituted heteroaralkyl;
      or independently of each other, each Rⁱ and R^{j}, R^{k} and R^{m}, Rⁿ and R^{o}, and R^{p} and R^{q}, together with the nitrogen atom to which they are attached form optionally substituted heterocyclyl;
R¹, R², R³, and R⁴ are independently selected from hydrogen, alkyl, cycloalkyl, halo, haloalkyl, haloalkoxy, alkoxy, cyano, hydroxy, hydroxylalkyl, amino, and aminoalkyl;
or one of R¹ and R², and R³ and R⁴, when attached to the same carbon, combine to form oxo, alkylidenyl, 3 to 6 membered cycloalkylene, or 4 to 6 membered optionally substituted heterocyclylene;
R⁵ and R⁶ are independently selected from hydrogen, alkyl, cycloalkyl, halo, haloalkyl, haloalkoxy, alkoxy, hydroxy, cyano, hydroxylalkyl, amino, and aminoalkyl, or wherein one of R⁵ and R⁶ is optionally substituted heterocyclyl and the other of R⁵ and R⁶ is selected from hydrogen, alkyl, cycloalkyl, halo, haloalkyl, haloalkoxy, alkoxy, hydroxy, cyano, hydroxylalkyl, amino, and aminoalkyl;
L is bond, O, S, S(O), S(O)₂, or CR⁷R⁸ where R⁷ and R⁸ are independently hydrogen or alkyl;
Z¹ is a group of formula (a) or (b):

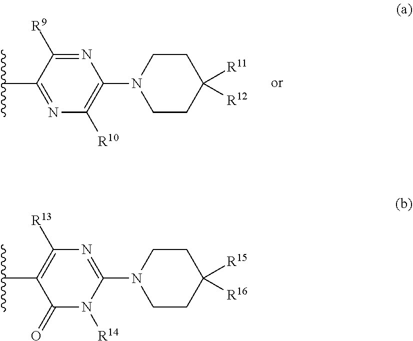

wherein:
R⁹ is hydrogen, alkyl, halo, hydroxy, amino, or haloalkyl;
R¹⁰ is hydrogen, alkyl, halo, hydroxy, hydroxyalkyl, —CD₂OH, alkylsulfoxide, alkylsulfonyl, amino, aminoalkyl, aminosulfonyl, aminocarbonyl, carboxy, cyano, or alkoxycarbonyl;
R¹³ is hydrogen, alkyl, halo, hydroxy, amino, or haloalkyl;
R¹⁴ is hydrogen, alkyl, or haloalkyl;
R¹¹ and R¹⁵ are selected from amino and aminoalkyl;
R¹² and R¹⁶ are selected from hydrogen, cyano, halo, alkyl, haloalkyl, alkoxy, haloalkoxy, cycloalkyl, aryl, heterocyclyl, and heteroaryl, where alkyl, cycloalkyl, aryl, heterocyclyl and heteroaryl are optionally substituted with one to three substituents independently selected from alkyl, halo, haloalkyl, haloalkoxy, alkoxy, and cyano;
or R¹¹ and R¹², and R¹⁵ and R¹⁶ together with the carbon atom to which they are attached form a ring of formula (c):

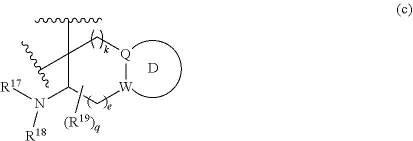

wherein:
e is 0, 1, or 2;
k is 0, 1, or 2 provided e+k is 1, 2, or 3;
q is 0, 1, or 2, or 3;
R¹⁷ and R¹⁸ are independently selected from hydrogen, alkyl, cycloalkyl and haloalkyl,
each R¹⁹ is independently selected from hydrogen, alkyl, halo, haloalkyl, haloalkoxy, alkoxy, hydroxy, cyano, alkylsulfoxide, alkylsulfonyl, oxo, cycloalkyl, optionally substituted heterocyclyl, and optionally substituted heteroaryl; or
when two R¹⁹ groups are attached to the same carbon atom, the two R¹⁹ groups together with the carbon atom to which they are attached form cycloalkylene or heterocyclylene.
ring D is absent or present; wherein:
   (i) when ring D is absent, then one of Q and W is CH₂, O, S, S(O), S(O)₂, or NH; and the other of Q and W is CH₂; and
   (ii) when ring D is present, then Q and W are independently N or C provided only one of Q and W is N; and ring D is phenyl or a 5 or 6 membered heteroaryl ring which, including Q and W, contains one to three heteroatoms independently selected from N, O, and S and ring D is optionally substituted with one or two substituents independently selected from alkyl, cycloalkyl, halo, haloalkyl, alkoxy, haloalkoxy, hydroxy, hydroxyalkyl, cyano, amino, aminoalkyl, carboxy, and optionally substituted heterocyclyl; or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments, when the compound of the formula (DK) immediately above, or a pharmaceutically acceptable salt thereof, is a compound of formula (DL):

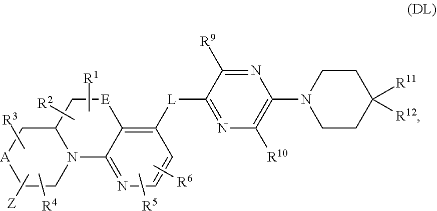

or a pharmaceutically acceptable salt thereof,
where R⁹ is hydrogen,
R¹⁰ is other than hydrogen, amino, and aminoalkyl, and
L, R¹¹ and R¹² are as defined in the embodiment immediately above; then:
   (i) when four of R¹, R², R³, R⁴, R⁵, and R⁶ are hydrogen and remaining two of R¹, R², R³, R⁴, R⁵, and R⁶, are independently selected from hydrogen, alkyl, cycloalkyl, amino, halo, haloalkyl, alkoxy, haloalkoxy, hydroxy, hydroxyalkyl, cyano, and aminoalkyl;
   then Z is other than hydrogen, alkyl, halo, haloalkyl, haloalkoxy, cyano, —ORₐ (where R^{a} is hydrogen or alkyl), —OC(O)NH₂, —O-tetrahydrofuran-3-yl, —O-oxetan-3-yl, cyano, pyrazol-1-yl, —CH2CH3, —OCH₂OCH₃, —OCH₂ cyclopropyl, —O—CH₂CH₂OCH₃, and —SO₂CH₃,
   (ii) when R⁵ and R⁶ are hydrogen and two of R¹, R², R³, and R⁴ are hydrogen, and one of a) R¹ and R² and b) R³ and R⁴, are hydrogen and the other of a) R¹ and R², and b) R³ and R⁴ are attached to the same carbon and are combined together to form alkylidene, 3 to 6 membered cycloalkylene or 4 to 6 membered heterocyclylene, then Z is other than hydrogen, alkyl, halo, haloalkyl, haloalkoxy, cyano, cycloalkyl, —ORₐ (where R^{a} is hydrogen or alkyl), —NH₂, and —Y-M (wherein Y is bond and M is alkyl substituted with —ORₐ or —NR^{p}R^{q} wherein each R^{a} is hydrogen or alkyl and R^{p} and R are independently hydrogen, alkyl, hydroxyalkyl or alkoxyalkyl or R^{p} and R^{q} together with the nitrogen atom to which they are attached form optionally substituted heterocyclyl); and
   (iii) when Z is hydrogen, one of a) R¹ and R², and b) R³ and R⁴ are attached to the same carbon and are combined together to form 3 to 6 membered cycloalkylene or 4 to 6 membered heterocyclylene, and three of the remaining R¹, R², R³, R⁴, R⁵, and R⁶ are hydrogen, then the remaining one of R¹, R², R³, R⁴, R⁵, and R⁶ is not hydrogen, alkyl, cycloalkyl, halo, haloalkyl, cyano, hydroxy, alkoxy, haloalkoxy, hydroxyalkyl, aminoalkyl, or amino;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments, the SHP2 inhibitor is a compound of having the formula (DM) immediately below, or a pharmaceutically acceptable salt or solvate thereof:

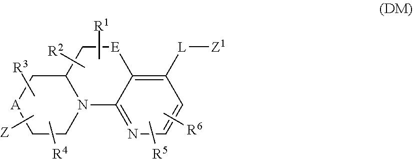

wherein:
A and E are independently selected from a bond, CH₂, O, NH, S, and S(O)₂;
Z is hydrogen, alkyl, haloalkyl, cyano, cycloalkyl, heterocyclyl, heteroaryl (wherein cycloalkyl, heterocyclyl, and heteroaryl are optionally substituted with one to three halo),
   —O(alk)_{y}R^{a}, —O(alk)OR^{b}, —S(O)R^{c}, —S(O)₂R^{d}, —NRC(O)R^{f}, —NR^{s}SO₂R^{h}, —OC(O)NRⁱR^{j}, —C(O)NR^{k}R^{m}, —S(O)₂NRⁿR^{o}, —NR^{p}R^{q}, —NR^{r}C(O)C(O)R^{s}
   or —Y-M
   wherein Y is bond, O, or SO₂ and
   M is alkyl, haloalkyl, cycloalkyl, heterocyclyl, or heteroaryl wherein alkyl, haloalkyl, cycloalkyl, heterocyclyl and heteroaryl are substituted with —O(alk)_{y}R^{a}, —O(alk)OR^{b}, —S(O)R^{c}, —S(O)₂R^{d}, —NRC(O)R, —NRₛSO₂R^{h}, —OC(O)NRⁱR^{j}, —C(O)NR^{k}R^{m}, —S(O)₂NRⁿR^{o}, —NR^{p}R^{q}, or —NR^{r}C(O)C(O)R^{s} and cycloalkyl, heterocyclyl, and heteroaryl are optionally further substituted with 1 to 3 halo;
      wherein each y is 0 or 1,
      each alk is alkylene, and
      each R^{c}, R^{d}, R^{f}, R^{h}, and R^{s} are independently alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; and
      each R^{a}, R^{b}, R^{e}, R^{g}, Rⁱ, R^{j}, R^{k}, R^{m}, Rⁿ, R^{o}, R^{p}, R^{q}, R^{r}, and R^{s} are independently hydrogen, alkyl, cycloalkyl, cycloalkylalkyl, hydroxyalkyl, alkoxyalkyl, aminoalkyl, optionally substituted heterocyclyl, optionally substituted heterocyclylalkyl, optionally substituted aryl, optionally substituted aralkyl, optionally substituted heteroaryl, or optionally substituted heteroaralkyl;
      or, independently of each other, each Rⁱ and R^{j}, R^{k} and R^{m}, Rⁿ and R^{o}, and R^{p} and R^{q}, together with the nitrogen atom to which they are attached form optionally substituted heterocyclyl;
R¹, R², R³, and R⁴ are independently selected from hydrogen, alkyl, cycloalkyl, halo, haloalkyl, haloalkoxy, alkoxy, cyano, hydroxy, hydroxylalkyl, amino, and aminoalkyl;
or one of R¹ and R² and R³ and R⁴, when attached to the same carbon, combine to form oxo, alkylidenyl, 3 to 6 membered cycloalkylene, or 4 to 6 membered optionally substituted heterocyclylene;
R⁵ and R⁶ are independently selected from hydrogen, alkyl, cycloalkyl, halo, haloalkyl, haloalkoxy, alkoxy, hydroxy, cyano, hydroxylalkyl, amino, and aminoalkyl, or wherein one of R⁵ and R⁶ is optionally substituted heterocyclyl and the other of R⁵ and R⁶ is selected from hydrogen, alkyl, cycloalkyl, halo, haloalkyl, haloalkoxy, alkoxy, hydroxy, cyano, hydroxylalkyl, amino, and aminoalkyl;
L is bond, O, S, S(O), S(O)₂, or CR⁷R⁸ where R⁷ and R⁸ are independently hydrogen or alkyl;
Z¹ is a group of formula (a) or (b):

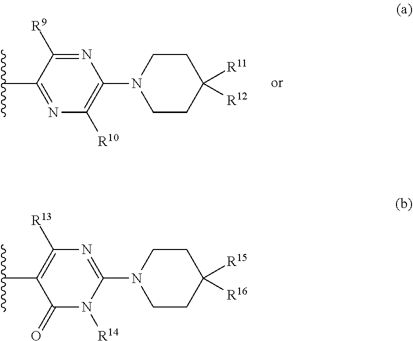

wherein:
   R⁹ is hydrogen, alkyl, halo, hydroxy, amino, or haloalkyl;
   R¹⁰ is hydrogen, alkyl, halo, hydroxy, hydroxyalkyl, —CD₂OH, alkylsulfoxide, alkylsulfonyl, amino, aminoalkyl, aminosulfonyl, aminocarbonyl, carboxy, cyano, or alkoxycarbonyl;
   R¹³ is hydrogen, alkyl, halo, hydroxy, amino, or haloalkyl;
   R¹⁴ is hydrogen, alkyl, or haloalkyl;
   R¹¹ and R¹⁵ are selected from amino and aminoalkyl;
   R¹² and R¹⁶ are selected from hydrogen, cyano, halo, alkyl, haloalkyl, alkoxy, haloalkoxy, cycloalkyl, aryl, heterocyclyl, and heteroaryl, where alkyl, cycloalkyl, aryl, heterocyclyl and heteroaryl are optionally substituted with one to three substituents independently selected from alkyl, halo, haloalkyl, haloalkoxy, alkoxy, and cyano;
   or R¹¹ and R¹², and R¹⁵ and R¹⁶ together with the carbon atom to which they are attached form a ring of formula (c):

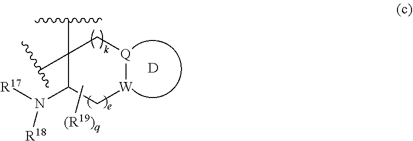

wherein:
e is 0, 1, or 2;
k is 0, 1, or 2 provided e+k is 1, 2, or 3;
q is 0, 1, or 2, or 3;
R¹⁷ and R¹⁸ are independently selected from hydrogen, alkyl, cycloalkyl and haloalkyl,
each R¹⁹ is independently selected from hydrogen, alkyl, halo, haloalkyl, haloalkoxy, alkoxy, hydroxy, cyano, alkylsulfoxide, alkylsulfonyl, oxo, cycloalkyl, optionally substituted heterocyclyl, and optionally substituted heteroaryl; or
when two R¹⁹ groups are attached to the same carbon atom, the two R¹⁹ groups together with the carbon atom to which they are attached form cycloalkylene or heterocyclylene.
ring D is absent or present; wherein:
   (i) when ring D is absent, then one of Q and W is CH₂, O, S, S(O), S(O)₂, or NH; and the other of Q and W is CH₂; and
   (ii) when ring D is present, then Q and W are independently N or C provided only one of Q and W is N; and ring D is phenyl or a 5 or 6 membered heteroaryl ring which, including Q and W, contains one to three heteroatoms independently selected from N, O, and S and ring D is optionally be substituted with one or two substituents independently selected from alkyl, cycloalkyl, halo, haloalkyl, alkoxy, haloalkoxy, hydroxy, hydroxyalkyl, cyano, amino, aminoalkyl, carboxy, and optionally substituted heterocyclyl;
or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments, when the compound of the formula (DM) immediately above, or a pharmaceutically acceptable salt thereof, is a compound of formula (DN):

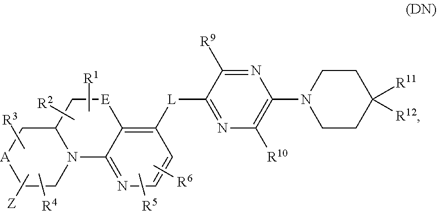

or a pharmaceutically acceptable salt thereof,
R⁹ is hydrogen,
R¹⁰ is other than hydrogen, amino, and aminoalkyl, and
L, R¹¹ and R¹² are as defined in the embodiment immediately above; then
   (i) when four of R¹, R², R³, R⁴, R⁵, and R⁶ are hydrogen and remaining two of R¹, R², R³, R⁴, R⁵, and R⁶ are independently selected from hydrogen, alkyl, cycloalkyl, halo, haloalkyl, alkoxy, haloalkoxy, hydroxy, hydroxyalkyl, cyano, amino, and aminoalkyl;
   then Z is other than hydrogen, halo, alkyl, haloalkyl, cyano, cycloalkyl, heterocyclyl, heteroaryl,
      wherein cycloalkyl, heterocyclyl, and heteroaryl are optionally substituted with one to three halo,
   —OR^{a}, —S(O)Re, —S(O)₂R^{d}, —NR^{e}C(O)R^{f}, —NR^{s}SO₂R^{h}, —OC(O)NRⁱR^{j}, —C(O)NR^{k}R^{m}, —S(O)₂NRⁿR^{o}, —NR^{p}R^{q}, —NR^{r}C(O)C(O)R^{s},
      wherein R^{c}, R^{d}, R^{f}, R^{h}, and R^{s} are independently alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; and R^{a}, R^{e}, R^{s}, Rⁱ, R^{j}, R^{k}, R^{m}, Rⁿ, R^{o}, R^{p}, R^{q}, and R^{r} are independently hydrogen, alkyl, cycloalkylalkyl, hydroxyalkyl, alkoxyalkyl, aminoalkyl, optionally substituted heterocyclyl, optionally substituted heterocyclylalkyl, optionally substituted aryl, optionally substituted aralkyl, optionally substituted heteroaryl, or optionally substituted heteroaralkyl; or, independently of each other, each Rⁱ and R^{j}, R^{k} and R^{m}, Rⁿ and R^{o}, and R^{p} and R^{q}, together with the nitrogen atom to which they are attached form optionally substituted heterocyclyl,
   and Y-M wherein
      (a) Y is bond or O and M is alkyl substituted with —OR^{a} or —NR^{p}R^{q} wherein R^{a} is hydrogen or alkyl and R^{p} and R^{q} are independently hydrogen, alkyl, hydroxyalkyl or alkoxyalkyl or R^{p} and R^{q} together with the nitrogen atom to which they are attached form optionally substituted heterocyclyl and
      (b) Y is SO₂ and M is cycloalkyl, substituted with —OR^{a},
      heteroaryl or heterocyclyl
      wherein heteroaryl or heterocyclyl are independently substituted with —ORₐ, —S(O)₂R^{d}, or —NR^{p}R^{q};
      where R^{a} is hydrogen or alkyl, R^{d} is alkyl, and R^{p} and R^{q} are independently hydrogen or alkyl and cycloalkyl is optionally further substituted with one halo and heterocyclyl, and heteroaryl are optionally further substituted with 1 or 2 halo;
   (ii) when R⁵ and R⁶ are each hydrogen and two of R¹, R², R³, and R⁴ are each hydrogen, and one of a) R¹ and R², and b) R³ and R⁴ are hydrogen and the other of a) R¹ and R², and b) R³ and R⁴ are attached to the same carbon and are combined together to form alkylidene, 3 to 6 membered cycloalkylene or 4 to 6 membered heterocyclylene, then Z is other than hydrogen, alkyl, halo, haloalkyl, cyano, cycloalkyl,
      —OR^{a}, wherein R^{a} is hydrogen or alkyl,
      —NH₂, and
      —Y-M, wherein Y is bond and M is alkyl substituted with —ORₐ or —NR^{p}R^{q}
         wherein each R^{a} is hydrogen or alkyl and R^{p} and R^{q} are independently hydrogen, alkyl, hydroxyalkyl or alkoxyalkyl or R^{p} and R^{q} together with the nitrogen atom to which they are attached form optionally substituted heterocyclyl); and
   (iii) when Z is hydrogen, one of a) R¹ and R², and b) R³ and R⁴ are attached to the same carbon and are combined together to form 3 to 6 membered cycloalkylene or 4 to 6 membered heterocyclylene and three of the remaining R¹, R², R³, R⁴, R⁵, and R⁶ are hydrogen, then the remaining one of R¹, R², R³, R⁴, R⁵, and R⁶ is not hydrogen, alkyl, halo, haloalkyl, cyano, cycloalkyl, hydroxy, alkoxy, haloalkoxy, hydroxyalkyl, aminoalkyl, or amino;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments, the SHP2 inhibitor is a compound of having the formula (DO) immediately below, or a pharmaceutically acceptable salt or solvate thereof:

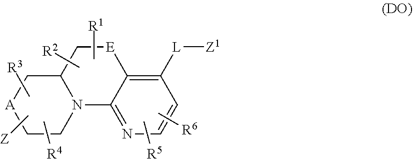

wherein:
A and E are independently selected from a bond, CH₂, O, NH, S, and S(O)₂;
Z is hydrogen, alkyl, halo, haloalkyl, haloalkoxy, cyano, cycloalkyl, heterocyclyl, heteroaryl,
   wherein cycloalkyl, heterocyclyl, and heteroaryl are optionally substituted with one to three halo,
   —O(alk)_{y}R^{a}, —O(alk)OR^{b}, —S(O)R^{c}, —S(O)₂R^{d}, —NR^{e}C(O)R^{f}, —NRₛSO₂R^{b}, —OC(O)NRⁱR^{j}, —C(O)NR^{k}R^{m}, —S(O)₂NRⁿR^{o}, —NR^{p}R^{q}, —NR^{r}C(O)C(O)R^{s},
   or —Y-M, wherein Y is bond, 0, or SO₂ and M is alkyl, haloalkyl, cycloalkyl, heterocyclyl, or heteroaryl
      wherein alkyl, haloalkyl, cycloalkyl, heterocyclyl and heteroaryl are substituted with —O(alk)_{y}R^{a}, —O(alk)OR^{b}, —S(O)R^{e}, —S(O)₂R^{d}, —NR^{e}C(O)R^{f}, —NR⁵SO₂R^{b}, —OC(O)NRⁱR^{j}, —C(O)NR^{k}R^{m}, —S(O)₂NRⁿR^{o}, —NR^{p}R^{q}, or —NR^{r}C(O)C(O)R^{s} and cycloalkyl, heterocyclyl, and heteroaryl are optionally further substituted with 1 to 3 halo;
   wherein each y is 0 or 1,
   each alk is alkylene, and
   each R^{c}, R^{d}, R^{f}, R^{h}, and R^{s} are independently alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; and
   each R^{a}, R^{b}, R^{e}, R^{g}, Rⁱ, R^{j}, R^{k}, R^{m}, Rⁿ, R^{o}, R^{p}, R^{q}, R^{r}, and R^{s} are independently hydrogen, alkyl, cycloalkyl, cycloalkylalkyl, hydroxyalkyl, alkoxyalkyl, aminoalkyl, optionally substituted heterocyclyl, optionally substituted heterocyclylalkyl, optionally substituted aryl, optionally substituted aralkyl, optionally substituted heteroaryl, or optionally substituted heteroaralkyl;
   or, independently of each other, each Rⁱ and R^{j}, R^{k} and R^{m}, Rⁿ and R^{o}, and R^{p} and R^{q}, together with the nitrogen atom to which they are attached form optionally substituted heterocyclyl;
R¹, R², R³, and R⁴ are independently selected from hydrogen, alkyl, cycloalkyl, halo, haloalkyl, haloalkoxy, alkoxy, cyano, hydroxy, hydroxylalkyl, amino, and aminoalkyl; or one of R¹ and R², and R³ and R⁴, when attached to the same carbon, combine to form oxo, alkylidenyl, 3 to 6 membered cycloalkylene, or 4 to 6 membered optionally substituted heterocyclylene;
R⁵ and R⁶ are independently selected from hydrogen, alkyl, cycloalkyl, halo, haloalkyl, haloalkoxy, alkoxy, hydroxy, cyano, hydroxylalkyl, amino, and aminoalkyl, or wherein one of R⁵ and R⁶ is optionally substituted heterocyclyl and the other is selected from hydrogen, alkyl, cycloalkyl, halo, haloalkyl, haloalkoxy, alkoxy, hydroxy, cyano, hydroxylalkyl, amino, and aminoalkyl;
L is bond, O, S, S(O), S(O)₂, or CR⁷R⁸ where R⁷ and R⁸ are independently hydrogen or alkyl;
Z¹ is a group of formula (a) or (b):

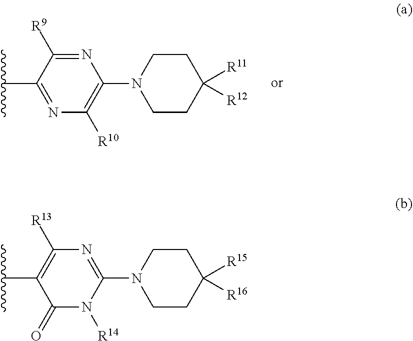

wherein:
   R⁹ is hydrogen, alkyl, halo, hydroxy, amino, or haloalkyl;
   R¹⁰ is hydrogen, alkyl, halo, hydroxy, hydroxyalkyl, —CD₂OH, alkylsulfoxide, alkylsulfonyl, amino, aminoalkyl, aminosulfonyl, aminocarbonyl, carboxy, cyano, or alkoxycarbonyl;
   R¹³ is hydrogen, alkyl, halo, hydroxy, amino, or haloalkyl;
   R¹⁴ is hydrogen, alkyl, or haloalkyl;
   R¹¹ and R¹⁵ are selected from amino and aminoalkyl;
   R¹² and R¹⁶ are selected from hydrogen, cyano, halo, alkyl, haloalkyl, alkoxy, haloalkoxy, cycloalkyl, aryl, heterocyclyl, and heteroaryl, where alkyl, cycloalkyl, aryl, heterocyclyl and heteroaryl are optionally substituted with one to three substituents independently selected from alkyl, halo, haloalkyl, haloalkoxy, alkoxy, and cyano;
   or R¹¹ and R¹², and R¹⁵ and R¹⁶ together with the carbon atom to which they are attached form a ring of formula (c):

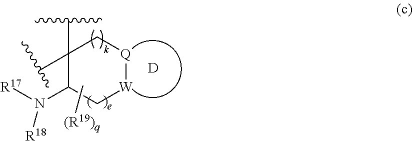

wherein:
e is 0, 1, or 2;
k is 0, 1, or 2 provided e+k is 1, 2, or 3;
q is 0, 1, or 2, or 3;
R¹⁷ and R¹⁸ are independently selected from hydrogen, alkyl, cycloalkyl, and haloalkyl;
each R¹⁹ is independently selected from hydrogen, alkyl, halo, haloalkyl, haloalkoxy, alkoxy, hydroxy, cyano, alkylsulfoxide, alkylsulfonyl, oxo, cycloalkyl, optionally substituted heterocyclyl, and optionally substituted heteroaryl; or
when two R¹⁹ groups are attached to the same carbon atom, the two R¹⁹ groups together with the carbon atom to which they are attached form cycloalkylene or heterocyclylene.
ring D is absent or present; wherein:
   (i) when ring D is absent, then one of Q and W is CH₂, O, S, S(O), S(O)₂, or NH; and the other of Q and W is CH₂; and
   (ii) when ring D is present, then Q and W are independently N or C provided only one of Q and W is N; and ring D is phenyl or a 5 or 6 membered heteroaryl ring which, including Q and W, contains one to three heteroatoms independently selected from N, O, and S and ring D is optionally substituted with one or two substituents independently selected from alkyl, cycloalkyl, halo, haloalkyl, alkoxy, haloalkoxy, hydroxy, hydroxyalkyl, cyano, amino, aminoalkyl, carboxy, and optionally substituted heterocyclyl; or a pharmaceutically acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments, the SHP2 inhibitor is a compound of having the formula (DP) immediately below, or a pharmaceutically acceptable salt or solvate thereof:

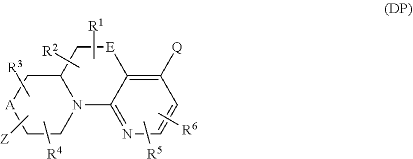

wherein:
Q is halo or SH;
A and E are independently selected from a bond, CH₂, O, NH, S, and S(O)₂;
Z is hydrogen, alkyl, halo, haloalkyl, haloalkoxy, cyano, cycloalkyl, heterocyclyl, heteroaryl,
   wherein cycloalkyl, heterocyclyl, and heteroaryl are optionally substituted with one to three halo,
   —O(alk)_{y}R^{a}, —O(alk)OR^{b}, —S(O)R^{e}, —S(O)₂R^{d}, —NR^{e}C(O)R^{f}, —NRₛSO₂R^{b}, —OC(O)NRⁱR^{j}, —C(O)NR^{k}R^{m}, —S(O)₂NRⁿR^{o}, —NR^{p}R^{q}, —NR^{r}C(O)C(O)R^{s},
   or —Y-M, wherein Y is bond, 0, or SO₂ and M is alkyl, haloalkyl, cycloalkyl, heterocyclyl, or heteroaryl
      wherein alkyl, haloalkyl, cycloalkyl, heterocyclyl and heteroaryl are substituted with —O(alk)_{y}R^{a}, —O(alk)_{y}OR^{b}, —S(O)R^{c}, —S(O)₂R^{d}, —NRC(O)R^{f}, —NRₛSO₂R^{h}, —OC(O)NRⁱR^{j}, —C(O)NR^{k}R^{m}, —S(O)₂NRⁿR^{o}, —NR^{p}R^{q}, or —NR^{r}C(O)C(O)R^{s} and cycloalkyl, heterocyclyl, and heteroaryl are optionally further substituted with 1 to 3 halo;
   wherein each y is 0 or 1,
   each alk is alkylene, and
   each R^{c}, R^{d}, R^{f}, R^{h}, and R^{s} are independently alkyl, cycloalkyl, cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; and
   each R^{a}, R^{b}, R^{e}, R^{g}, Rⁱ, R^{j}, R^{k}, R^{m}, Rⁿ, R^{o}, R^{p}, R^{q}, R^{r}, and R^{s} are independently hydrogen, alkyl, cycloalkyl, cycloalkylalkyl, hydroxyalkyl, alkoxyalkyl, aminoalkyl, optionally substituted heterocyclyl, optionally substituted heterocyclylalkyl, optionally substituted aryl, optionally substituted aralkyl, optionally substituted heteroaryl, or optionally substituted heteroaralkyl;
   or, independently of each other, each Rⁱ and R^{j}, R^{k} and R^{m}, Rⁿ and R^{o}, and R^{p} and R^{q}, together with the nitrogen atom to which they are attached form optionally substituted heterocyclyl;
R¹, R², R³, and R⁴ are independently selected from hydrogen, alkyl, cycloalkyl, halo, haloalkyl, haloalkoxy, alkoxy, cyano, hydroxy, hydroxylalkyl, amino, and aminoalkyl; or one of R¹ and R², and R³ and R⁴, when attached to the same carbon, combine to form oxo, alkylidenyl, 3 to 6 membered cycloalkylene, or 4 to 6 membered optionally substituted heterocyclylene;
R⁵ and R⁶ are independently selected from hydrogen, alkyl, cycloalkyl, halo, haloalkyl, haloalkoxy, alkoxy, hydroxy, cyano, hydroxylalkyl, amino, and aminoalkyl, or wherein one of R⁵ and R⁶ is optionally substituted heterocyclyl and the other of R⁵ and R⁶ is selected from hydrogen, alkyl, cycloalkyl, halo, haloalkyl, haloalkoxy, alkoxy, hydroxy, cyano, hydroxylalkyl, amino, and aminoalkyl; or an acceptable salt thereof;
as used in this embodiment, “Aryl” means a monovalent monocyclic or bicyclic aromatic hydrocarbon radical of 6 to 10 ring atoms e.g., phenyl or naphthyl. As used in this embodiment, “Cycloalkyl” means a monocyclic saturated monovalent hydrocarbon radical of three to ten carbon atoms optionally substituted with one or two substituents independently selected from alkyl, halo, alkoxy, hydroxy, and cyano, unless stated otherwise. As used in this embodiment, “Cycloalkyl fused bicyclic heteroaryl” means a bicyclic heteroaryl, as defined in this paragraph, containing 9 or 10 rings atoms that is fused to a 5 or 6-membered cycloalkyl ring, as defined in this embodiment, and which is attached to the remainder of the compound through the 5- or 6-membered heteroaryl ring portion of the bicyclic heteroaryl ring. As used in this embodiment, “Fused heteroaryl” means a bicyclic or tricyclic ring wherein a heteroaryl ring is fused to a heterocyclyl ring, each ring as defined in this embodiment. As used in this embodiment, “Heterocyclyl” means a saturated or unsaturated monovalent monocyclic or bicyclic ring of 4 to 10 ring atoms in which one, two, or three ring atoms are heteroatom selected from N, O, and S(O)ₙ, where n is an integer from 0 to 2, the remaining ring atoms being C. Additionally, one or two ring carbon atoms in the heterocyclyl ring of this embodiment can optionally be replaced by a —CO— group. When a heterocyclyl ring of this embodiment is unsaturated it can contain one or two ring double bonds provided that the ring is not aromatic. When a heterocyclyl group of this embodiment contains at least one nitrogen atom, it is also referred to herein as heterocycloamino and is a subset of the heterocyclyl group. As used in this embodiment, “Heteroaryl” means a monovalent monocyclic or bicyclic aromatic radical of 5 to 10 ring atoms, unless otherwise stated, where one or more, (e.g., one, two, or three), ring atoms are heteroatom selected from N, O, and S, the remaining ring atoms being carbon. As used in this embodiment, the terms“heteroaryl” and“aryl” are mutually exclusive. When the heteroaryl ring of this embodiment contains 5- or 6 ring atoms it is also referred to herein as 5- or 6-membered heteroaryl.

In embodiments the SHP2 inhibitor is a compound of having the formula (DQ) immediately below, or a pharmaceutically acceptable salt or solvate thereof:

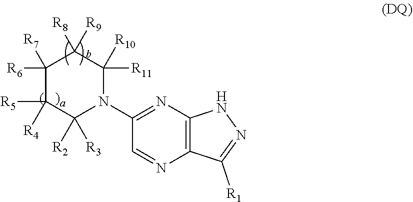

or a salt or tautomer thereof, wherein:
a is selected from 0 and 1;
b is selected from 0 and 1;
R₁ is selected from halo, C₆₋₁₀aryl, C₃₋₈cycloalkyl, C₃₋₈cycloalkenyl, and a 5-9 membered heteroaryl group containing 1 to 4 heteroatoms or groups independently selected from N, C(O), O, and S;
   said aryl or heteroaryl of R₁ is optionally substituted with 1 to 5 R₁₂ groups independently selected from halo, hydroxy, amino, C₁₋₄ alkylamino, C₁₋₄dialkylamino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₃, NR₁₅C(O)OR₁₃, NR₁₃C(O)NR₁₅R₁₆, NR₁₅S(O)R₁₃, NR₁₅S(O)₂R₁₃, C(O)NR₁₅R₁₆, S(O)NR₁₅R₁₆, S(O)₂NR₁₅R₁₆, C(O)R₁₃, C(O)OR₁₃, SR₁₃, S(O)R₁₃, and S(O)₂R₁₃,
R₂, R₃, R₁₀, and R₁₁ are independently selected from hydrogen, C₁₋₄alkyl, and C₃₋₈cycloalkyl;
R⁴, R₅, R₈, and R₉ are independently selected from hydrogen, cyano, C₁₋₄alkyl, C₁₋₄alkoxy, amino, hydroxy, C₃₋₈cycloalkyl, halo, and C₁₋₄alkylamino;
R₆ is selected from amino, C₁₋₄aminoalkyl, and C₁₋₄alkylamino;
R₇ is selected from hydrogen, cyano, amido, halo, and hydroxy, or is selected from C₁₋₄alkyl, C₁₋₄hydroxyalkyl, C₃₋₆cycloalkyl, phenyl, and 5- or 6-membered heteroaryl, any of which is optionally substituted with one or more R₁₇ groups;
   or R₆ and R₇ together with the carbon atom to which they are both attached form a 3- to 7-membered saturated or unsaturated ring that can contain 1 to 3 heteroatoms or groups independently selected from N, C(O), O, and S(O)ₘ, and that is optionally substituted with one R¹⁷ group, and that is optionally substituted with one or more R₁₈ groups;
m is selected from 0, 1, and 2;
   any two groups selected from R₂, R₃, R₄, R₅, R₇, R₈, R₉, R₁₀ and R₁₁ can form a 5- to 6-membered ring, optionally containing a N, O or S heteroatom; any two groups selected from R₂, R₄, R₆, R₈ and R₁₀ can form a direct bond, or a 1 or 2 atom carbon bridge;
   R₁₃, R₁₅, and R₁₆ are independently selected from hydrogen, C₁₋₄alkyl, and
   C₃₋₈cycloalkyl, wherein said alkyl or cycloalkyl is optionally substituted by one or more substituents selected from hydroxy, cyano and halo; and
   each R₁₇ and R₁₈ is independently selected from amino, halo, hydroxy, cyano, trifluoromethyl, trifluoromethoxy, C₁₋₄alkyl, and C₁₋₄alkoxy;
as used in this embodiment, the term “aryl,”, alone or in combination, means a carbocyclic aromatic system containing one, two or three rings wherein such polycyclic ring systems are fused together. As used in this embodiment, the term “aryl” embraces aromatic groups such as phenyl, naphthyl, anthracenyl, and phenanthryl. As used in this embodiment, the term “cycloalkyl,” or, alternatively, “carbocycle,” alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. As used in this embodiment, the term “heteroalkyl,” alone or in combination, refers to a stable straight or branched chain, or combinations thereof, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. As used in this embodiment, the heteroatom(s) may be placed at any interior position of the heteroalkyl group. As used in this embodiment, the term “heteroaryl,” alone or in combination, refers to a 3 to 15 membered unsaturated heteromonocyclic ring, or a fused monocyclic, bicyclic, or tricyclic ring system in which at least one of the fused rings is aromatic, which contains at least one atom selected from N, O, and S; the term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. As used in this embodiment, the terms“heterocycloalkyl” and, interchangeably, “heterocycle,” alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. As used in this embodiment, “heterocycloalkyl” and“heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined in this paragraph, or an additional heterocycle group.

In embodiments the SHP2 inhibitor is a compound of having the formula (DR) immediately below, or a pharmaceutically acceptable salt or solvate thereof:

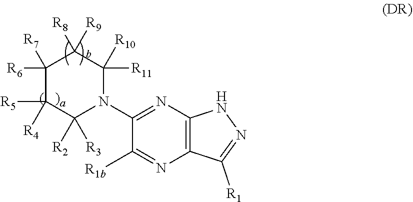

or a salt or tautomer thereof, wherein:
a is 0 or 1;
b is 0 or 1;
R₁ₐ is selected from the group consisting of halo, C₆₋₁₀aryl, C₃₋₈cycloalkyl, C₃₋₈cycloalkenyl, and a 5-9 membered heteroaryl group containing 1 to 4 heteroatoms or groups independently selected from the group consisting of N, C(O), 0, and S;
   said aryl or heteroaryl of R₁ₐ is optionally substituted with 1 to 5 R₁₂ groups independently selected from the group consisting of halo, hydroxy, amino, C₁₋₄alkylamino, C₁₋₄dialkylamino, cyano, C₁₋₄alkyl, C₁₋₄alkoxy, C₁₋₄hydroxyalkyl, C₁₋₄dihydroxyalkyl, hydroxyC₁₋₄alkoxy, dihydroxyC₁₋₄alkoxy, C₁₋₄haloalkyl, C₁₋₄aminoalkyl, C₃₋₈cycloalkyl, C₃₋₈cycloalkenyl, NR₁₅C(O)R₁₃, NR₁₅C(O)OR₁₃, NR₁₃C(O)NR₁₅R₁₆, NR₁₅S(O)R₁₃, NR₁₅S(O)₂R₁₃, C(O)NR₁₅R₁₆, S(O)NR₁₅R₁₆, S(O)₂NR₁₅R₁₆, C(O)R₁₃, C(O)OR₁₃, OR₁₃, SR₁₃, S(O)R₁₃, and S(O)₂R₁₃;
R_{1b} is selected from the group consisting of halogen, cyano, C₁₋₆alkyl, C₁₋₆haloalkyl C₁₋₆hydroxyalkyl, C₁₋₆dihydroxyalkyl, —CF₂OH, —CHFOH, —NH—NHR₁₉, —NH—OR₁₉, —O—NR₁₉R₂₀, —NHR₁₉, —OR₁₉, —NHC(O)R₁₉, —NHC(O)NHR₁₉, —NHS(O)₂NHR₁₉, —NHS(O)₂R₁₉, —C(O)OR₁₉, —C(O)NR₁₉R₂₀, —C(O)NH(CH₂)ₙOH, —C(O)NH(CH₂)ₙR₂₁, —C(O)R₂₁, —NH₂, —OH, —CN, —S(O)₂NR₁₉R₂₀, C₃-C₈cycloalkyl, aryl, heterocyclyl having 1-5 heteroatom ring vertices selected from the group consisting of N, O, S and P, heteroaryl having 1-5 heteroatom ring vertices selected from the group consisting of N, O, S and P; wherein the subscript n is an integer of from 0 to 6; and
   wherein aryl, heteroaryl, heterocyclyl and cycloalkyl are substituted with 0 to 3 members independently selected from the group consisting of C₁₋₄alkyl, —OH, —NH₂, —OR₂₁, halogen, cyano and oxo;
R₂, R₃, R₁₀, and R₁₁ are independently selected from the group consisting of hydrogen, C₁₋₄alkyl, and C₃₋₈cycloalkyl;
R₄, R₅, R₈, and R₉ are independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, cyano, amido, halo, and hydroxy, or is selected from the group consisting of C₁₋₄ alkyl, C₁₋₄hydroxyalkyl, C₃₋₈cycloalkyl, phenyl, and 5- or 6-membered heteroaryl, any of which is optionally substituted with one or more R₁₇ groups;
or R₆ and R₇ together with the carbon atom to which they are both attached form a 3- to 7-membered saturated or unsaturated ring that can contain 1 to 3 heteroatoms or groups independently selected from the group consisting of N, C(O), O, and S(O)ₘ, and that is optionally substituted with one R₁₇ group, and that is optionally substituted with one or more R₁₈ groups;
m is 0, 1, or 2;
any two groups of R₂, R₃, R₄, R₅, R₇, R₈, R₉, R₁₀ and R₁₁ can form a 5- to 6-membered ring, optionally containing a N, O or S heteroatom;
any two groups of R₂, R₄, R₆, R₈ and R₁₀ can form a direct bond, or a 1 or 2 atom carbon bridge;
   R₁₃, R₁₅, and R₁₆ are independently selected from the group consisting of hydrogen, C₁₋₄alkyl, C₃₋₈cycloalkyl, and 3- to 6-membered heterocyclyl, wherein said alkyl, cycloalkyl and 3- to 6-membered heterocyclyl is optionally substituted by one or more substituents selected from the group consisting of hydroxy, cyano and halo;
   each R₁₇ and R₁₈ is independently selected from the group consisting of amino, halo, hydroxy, cyano, trifluoromethyl, trifluoromethoxy, C₁₋₄alkyl, and C₁₋₄alkoxy;
   each R₁₉ and R₂₀ is independently selected from the group consisting of H, C₁₋₆alkyl, C₁₋₆haloalkyl, C₁₋₆hydroxyalkyl, C₂₋₆alkenyl, C₂₋₆alkynyl and C₃₋₆cycloalkyl;
   and each R²¹ is independently selected from the group consisting of H, —OH, C₁₋₆alkyl, C₁₋₆haloalkyl, C₁₋₆hydroxyalkyl, C₂₋₆alkenyl, C₂₋₆alkynyl and C₃₋₆cycloalkyl;
as used in this embodiment, the term “alkyl,” alone or in combination, refers to a straight-chain or branched-chain alkyl radical. As used in this embodiment, unless otherwise specified, the term “alkyl” may include “alkylene” groups. As used in this embodiment, the term “alkenyl,” alone or in combination, refers to a straight-chain or branched-chain hydrocarbon radical having one or more double bonds. As used in this embodiment, the term “aryl,” alone or in combination, means a carbocyclic aromatic system containing one, two or three rings wherein such polycyclic ring systems are fused together. As used in this embodiment, the term “cycloalkyl,” or, alternatively, “carbocycle,” alone or in combination, refers to a saturated or partially saturated monocyclic, bicyclic or tricyclic alkyl group, which may optionally be a benzo fused ring system which is optionally substituted as defined herein. As used in this embodiment, the term “cycloalkenyl” refers to a cycloalkyl group having one or two double bonds. As used in this embodiment, the term “heteroalkyl,” alone or in combination, refers to a stable straight or branched chain, or combinations thereof, fully saturated or containing from 1 to 3 degrees of unsaturation, consisting of the stated number of carbon atoms and from one to three heteroatoms selected from the group consisting of N, O, and S, and wherein the N and S atoms may optionally be oxidized and the N heteroatom may optionally be quaternized, the heteroatom(s) may be placed at any interior position of the heteroalkyl group. As used in this embodiment, the term “heteroalkyl,” alone or in combination, refers to a stable straight or branched chain, or combinations thereof, fully saturated or containing from 1 to 3 degrees of unsaturation, consisting of the stated number of carbon atoms and from one to three heteroatoms selected from the group consisting of N, O, and S, and wherein the N and S atoms may optionally be oxidized and the N heteroatom may optionally be quaternized, the heteroatom(s) may be placed at any interior position of the heteroalkyl group. As used in this embodiment, the terms “heterocycloalkyl” and, interchangeably, “heterocycle,” alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from the group consisting of nitrogen, oxygen, and sulfur. As used in this embodiment, “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as used in this embodiment, or an additional heterocycle group.

In embodiments, the compound is

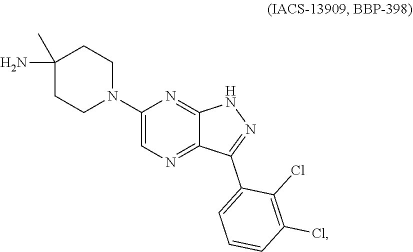 [SMILES: CC1(N)CCN(c2cnc3c(-c4cccc(Cl)c4Cl)n[nH]c3n2)CC1]

or a pharmaceutically acceptable salt or solvate thereof.

In embodiments the SHP2 inhibitor is a compound of having the formula (DS) immediately below, or a pharmaceutically acceptable salt or solvate thereof:

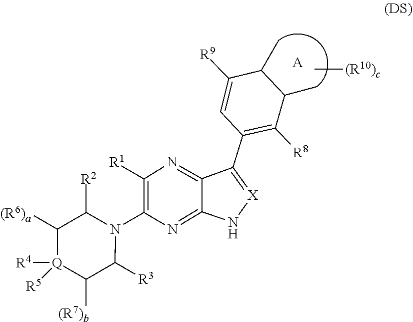

or a tautomer or a solvate or a pharmaceutically acceptable salt thereof, wherein:
X is CH or N;
R¹ is hydrogen, —CH₃ or —CH₂OH but when X is N then R¹ is selected from —CH₃ and —CH₂OH;
R² and R³ are either:
   (i) independently selected from hydrogen and C₁₋₄alkyl; or
   (ii) together form a one- to three-membered bridge group selected from C₁₋₃alkylene, C₂₋₃alkenylene, methylene-NR^{q}-methylene and methylene-O-methylene, wherein the bridge group is optionally substituted by a group selected from C₁₋₄alkyl, hydroxyl and halogen and R^{q} is selected from hydrogen, C₁₋₄alkyl, hydroxyl and halogen;
Q is C or N;
wherein when Q is C then either:
   (i) R⁴ is hydrogen or C₁₋₄alkyl (e.g. methyl) optionally substituted by amino (e.g. —CH₂NH₂);
   R⁵ is hydrogen, amino, hydroxyl or C₁₋₄alkyl (e.g. methyl) optionally substituted by 1 or 2 groups selected from halogen, hydroxyl (e.g. —CH₂OH) or amino;
   provided that R⁴ and R⁵ must not both be selected from amino and C₁₋₄alkyl substituted by amino; or
   (ii) R⁴ and R⁵ together with Q form a four- to six-membered nitrogen-containing heterocyclic ring; and
wherein when Q is N then:
   R⁴ is absent;
   R⁵ is hydrogen; and
   R² and R³ together form the one- to three-membered bridge group;
R⁶ and R⁷ are independently selected from halogen (e.g. fluorine), C₁₋₄alkyl (e.g. —CH₃) and hydroxyl provided that when Q is N then
R⁶ or R⁷ are not halogen or hydroxyl;
a is selected from 0, 1 and 2;
b is selected from 0, 1 and 2;
Ring A is either:
   (i) a five-membered nitrogen-containing heterocyclic ring (e.g. an aromatic ring or a non-aromatic ring) wherein the heterocyclic ring optionally contains one or two additional heteroatoms selected from N, O and S, or
   (ii) a six-membered aromatic nitrogen-containing heterocyclic ring, wherein the heterocyclic ring optionally contains one or two additional heteroatoms selected from N, O and S; or
   (iii) a six-membered non-aromatic nitrogen-containing heterocyclic ring, wherein the heterocyclic ring optionally contains one or two additional heteroatoms selected from N and S;
R⁸ is selected from haloC₁₋₄alkyl (e.g. —CF₃), —CH₃ and halogen (e.g. chlorine or fluorine);
R⁹ is selected from hydrogen, C₁₋₄alkyl (e.g. —CH₃), haloC₁₋₄alkyl (e.g. —CF₃) and halogen (e.g. chlorine); R¹⁰ are independently selected from halogen, cyano, cyanoC₁₋₄alkyl (e.g. —CH₂—CN), hydroxyl, ═O (oxo), C₁₋₄alkyl (e.g. —CH₃, —CH₂CH₃, and —CH(CH₃)₂), haloC₁₋₄alkyl (e.g. —CHF₂), C₁₋₄alkoxy (e.g. —OCH₃, —OCH₂CH₃ and —OCH(CH₃)₂), hydroxylC₁₋₄alkyl (e.g. —CH₂C(CH₃)₂OH, —CH(CH₃)CH₂OH, —CH(CH₃)OH, —CH₂CH₂OH or —CH₂OH), C₁₋₄alkoxyC₁₋₄alkylene (e.g. —CH₂—O—CH₃ or —CH₂—CH₂—O—CH₃), C₁₋₄alkylsulfone (e.g. —SO₂CH₃), amino, monoC₁₋₄alkylamino, diC₁₋₄alkylamino (e.g. —N(CH₃)₂), aminoC₁₋₄alkylene (e.g. —CH₂NH₂), —C₀₋₄alkylene-C(═O)NH_{(2-q)}(C₁₋₆alkyl)_{q}), —C₁₋₄alkylene-NHC(═O)C₁₋₆alkyl, sulfonamideC₀₋₄alkylene (e.g. —SO₂NR₂ or —CH₂SO₂NR′₂), wherein H is independently selected from H and C₁₋₆alkyl), 3 to 6 membered cycloalkyl, optionally substituted five- or six-membered unsaturated heterocyclic group containing 1, 2, 3 or 4 heteroatoms selected from O, N, or S where the optional substituent is selected from C₁₋₄alkyl, C₁₋₄alkyl substituted with 3 to 6 membered cycloalkyl, C₁₋₄alkyl substituted with optionally substituted five- or six-membered unsaturated heterocyclic group containing 1, 2, 3 or 4 heteroatoms selected from O, N, or S where the optional substituent is selected from C₁₋₄alkyl, C₁₋₄alkyl substituted with optionally substituted four- to six-membered saturated heterocyclic group containing 1 or 2 heteroatoms selected from O, N, or S where the optional substituent is selected from C₁₋₄alkyl and optionally substituted four- to six-membered saturated heterocyclic group containing 1 or 2 heteroatoms selected from O, N, or S where the optional substituent is selected from C₁₋₄alkyl; q is selected from 0, 1 or 2; and
c is selected from 0, 1, 2 and 3.

In a second aspect, the invention provides a compound of formula (I), or a tautomer, N-oxide, pharmaceutically acceptable salt or solvate thereof, wherein:
X is CH or N;
R¹ is hydrogen, —CH₃ or —CH₂OH but when X is N then R¹ is selected from —CH₃ and —CH₂OH;
R² and R³ are either:
   (i) independently selected from hydrogen and C₁₋₄alkyl; or
   (ii) together form a one- to three-membered bridge group selected from C₁₋₃alkylene, C₂₋₃alkenylene, methylene-NR′-methylene and methylene-O-methylene, wherein the bridge group is optionally substituted by a group selected from C₁₋₄alkyl, hydroxyl and halogen and R′ is selected from hydrogen, C₁₋₄alkyl, hydroxyl and halogen.
Q is C or N;
   wherein when Q is C then either: (i) R⁴ is hydrogen or C₁₋₄alkyl (e.g. methyl) optionally substituted by amino (e.g. —CH₂NH₂);
   R⁵ is hydrogen, amino, or C₁₋₄alkyl (e.g. methyl) optionally substituted by 1 or 2 groups selected from halogen, hydroxyl (e.g. —CH₂OH) or amino;
   provided that R⁴ and R⁵ must not both be selected from amino and C₁₋₄alkyl substituted by amino; or
   (ii) R⁴ and R⁵ together with Q form a four- to six-membered nitrogen-containing heterocyclic ring; and
   wherein when Q is N then:
   R⁴ is absent;
   R⁵ is hydrogen; and
   R² and R³ together form the one- to three-membered bridge group;
R⁶ and R⁷ are independently selected from halogen (e.g. fluorine), C₁₋₄alkyl (e.g. —CH₃) and hydroxyl provided that when Q is N then R⁶ or R⁷ are not halogen or hydroxyl;
a is selected from 0, 1 and 2;
b is selected from 0, 1 and 2;
Ring A is either:
   (i) a five-membered nitrogen-containing heterocyclic ring (e.g. an aromatic ring or a non-aromatic ring) wherein the heterocyclic ring optionally contains one or two additional heteroatoms selected from N, O and S, or
   (ii) a six-membered aromatic nitrogen-containing heterocyclic ring, wherein the heterocyclic ring optionally contains one or two additional heteroatoms selected from N, O and S; or
   (iii) a six-membered non-aromatic nitrogen-containing heterocyclic ring, wherein the heterocyclic ring optionally contains one or two additional heteroatoms selected from N and S;
R⁸ is selected from haloC₁₋₄alkyl (e.g. —CF₃), —CH₃ and halogen (e.g. chlorine or fluorine);
R⁹ is selected from hydrogen, C₁₋₄alkyl (e.g. —CH₃), haloC₁₋₄alkyl (e.g. —CF₃) and halogen (e.g. chlorine); R¹⁰ are independently selected from halogen, cyano, cyanoC₁₋₄alkyl (e.g. —CH₂—CN), hydroxyl, ═O (oxo), C₁₋₄alkyl (e.g. —CH₃ or —CH₂CH₃), haloC₁₋₄alkyl, C₁₋₄ alkoxy (e.g. —OCH₃), hydroxylC₁₋₄alkyl (e.g. —CH₂C(CH₃)₂OH, —CH(CH₃)CH₂OH, —CH(CH₃)OH, —CH₂CH₂OH or —CH₂OH), C₁₋₄ alkoxyC₁₋₄alkylene (e.g. —CH₂—O—CH₃ or —CH₂—CH₂—O—CH₃), C₁₋₄alkylsulfone (e.g. —SO₂CH₃), amino, monoC₁₋₄alkylamino, diC₁₋₄ alkylamino (e.g. —N(CH₃)₂), aminoC₁₋₄alkylene (e.g. —CH₂NH₂), —C₁₋₄alkylene-C(═O)NH_{(2-q)}(C₁₋₆alkyl)_{q}), —C₁₋₄alkylene-NHC(═O)C₁₋₆ alkyl, sulfonamideC₀₋₄alkylene (e.g. —SO₂NR₂ or —CH₃SO₂NR₂, wherein R^{x} is independently selected from H and C₁₋₆alkyl), and optionally substituted four- to six-membered saturated heterocyclic group containing 1 or 2 heteroatoms selected from O, N, or S where the optional substituent is selected from C₁₋₄alkyl;
q is selected from 0, 1 or 2; and
c is selected from 0, 1 and 2;
as used in this embodiment, the term ‘cycloalkyl’ refers to a saturated monocyclic hydrocarbon ring. As used in this embodiment, the term ‘cycloalkenyl’ refers to a partially saturated monocyclic hydrocarbon ring having one or more (usually one) carbon carbon double bond(s). As used in this embodiment, the term “heterocyclyl group” shall unless the context indicates otherwise, include both aromatic and non-aromatic ring systems. As used in this embodiment, the term “heterocyclyl group” include within their scope aromatic, non-aromatic, unsaturated, partially saturated and saturated heterocyclyl ring systems, in general, unless the context indicates otherwise, such groups may be monocyclic or bicyclic (including fused, spiro and bridged bicyclic groups) As used in this embodiment, the heterocyclyl groups can be heteroaryl groups, the heterocyclyl ring can, unless the context indicates otherwise, be optionally substituted i.e. unsubstituted or substituted As used in this embodiment, the term “heteroaryl” denotes a heterocyclyl group having aromatic character, the term “heteroaryl” embraces polycyclic (e.g. bicyclic) ring systems wherein one or more rings are nonaromatic, provided that at least one ring is aromatic, and in such polycyclic systems, the group may be attached by the aromatic ring, or by a non-aromatic ring to the remainder of the compound. As used in this embodiment, the term “non-aromatic” embraces, unless the context indicates otherwise, unsaturated ring systems without aromatic character, partially saturated and saturated heterocyclyl ring systems, wherein the terms “unsaturated” and“partially saturated” refer to rings wherein the ring structure(s) contains atoms sharing more than one valence bond i.e. the ring contains at least one multiple bond e.g. a C═C, C° C. or N═C bond; the heterocyclyl groups can be polycyclic fused ring systems or bridged ring systems such as the oxa- and aza analogues of bicycloalkanes, tricycloalkanes.

In embodiments the SHP2 inhibitor is a compound of having the formula (DT) immediately below, or a pharmaceutically acceptable salt or solvate thereof:

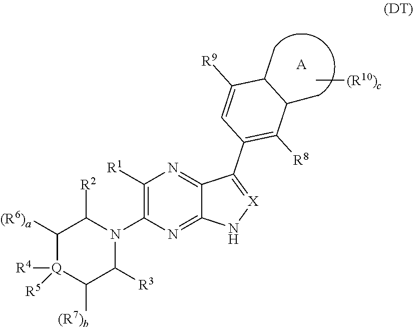

or a tautomer or a solvate or a pharmaceutically acceptable salt thereof,
wherein:
X is CH or N;
R¹ is hydrogen, —CH₃ or —CH₂OH but when X is N then R¹ is selected from —CH₃ and —CH₂OH;
R² and R³ are either:
   (i) independently selected from hydrogen and C₁₋₄alkyl; or
   (ii) together form a one- to three-membered bridge group selected from C₁₋₃alkylene, C₂₋₃alkenylene, methylene-NR^{q}-methylene and methylene-O-methylene, wherein the bridge group is optionally substituted by a group selected from C₁₋₄alkyl, hydroxyl and halogen and R^{q} is selected from hydrogen, C₁₋₄alkyl, hydroxyl and halogen;
Q is C or N;
wherein when Q is C then either:
   (i) R⁴ is hydrogen or C₁₋₄alkyl (e.g. methyl) optionally substituted by amino (e.g. —CH₂NH₂);
   R⁵ is hydrogen, amino, hydroxyl or C₁₋₄alkyl (e.g. methyl) optionally substituted by 1 or 2 groups selected from halogen, hydroxyl (e.g. —CH₂OH) or amino;
   provided that R⁴ and R⁵ must not both be selected from amino and C₁₋₄alkyl substituted by amino; or
   (ii) R⁴ and R⁵ together with Q form a four- to six-membered nitrogen-containing heterocyclic ring; and
   wherein when Q is N then:
   R⁴ is absent;
   R⁵ is hydrogen; and
   R² and R³ together form the one- to three-membered bridge group;
R⁶ and R⁷ are independently selected from halogen (e.g. fluorine), C₁₋₄alkyl (e.g. —CH₃) and hydroxyl provided that when Q is N then
R⁶ or R⁷ are not halogen or hydroxyl;
a is selected from 0, 1 and 2;
b is selected from 0, 1 and 2;
Ring A is either:
   (i) a five-membered nitrogen-containing heterocyclic ring (e.g. an aromatic ring or a non-aromatic ring) wherein the heterocyclic ring optionally contains one or two additional heteroatoms selected from N, O and S, or
   (ii) a six-membered aromatic nitrogen-containing heterocyclic ring, wherein the heterocyclic ring optionally contains one or two additional heteroatoms selected from N, O and S; or
   (iii) a six-membered non-aromatic nitrogen-containing heterocyclic ring, wherein the heterocyclic ring optionally contains one or two additional heteroatoms selected from N and S;
R⁸ is selected from haloC₁₋₄alkyl (e.g. —CF₃), —CH₃ and halogen (e.g. chlorine or fluorine);
R⁹ is selected from hydrogen, C₁₋₄alkyl (e.g. —CH₃), haloC₁₋₄alkyl (e.g. —CF₃) and halogen (e.g. chlorine); R¹⁰ are independently selected from halogen, cyano, cyanoC₁₋₄alkyl (e.g. —CH₂—CN), hydroxyl, ═O (oxo), C₁₋₄alkyl (e.g. —CH₃ and —CH₂CH₃), haloC₁₋₄ alkyl, C₁₋₄alkoxy (e.g. —OCH₃), hydroxylC₁₋₄alkyl (e.g. —CH₂C(CH₃)₂OH, —CH(CH₃)CH₂OH, —CH(CH₃)OH, —CH₂CH₂OH or —CH₂OH), C₁₋₄alkoxyC₁₋₄alkylene (e.g. —CH₂—O—CH₃ or —CH₂—CH₂—O—CH₃), C₁₋₄alkylsulfone (e.g. —SO₂CH₃), amino, monoC₁₋₄ alkylamino, diC₁₋₄alkylamino (e.g. —N(CH₃)₂), aminoC₁₋₄alkylene (e.g. —CH₂NH₂), —C₁₋₄alkylene-C(═O)NH_{(2-q)}(C₁₋₆alkyl)_{q}), —C₁₋₄ alkylene-NHC(═O)C₁₋₆alkyl, sulfonamideC₀₋₄alkylene (e.g. —SO₂NR₂ or —CH₃SO₂NR′₂, wherein R¹ is independently selected from H and C₁₋₆alkyl), 3 to 6 membered cycloalkyl, optionally substituted five- or six-membered unsaturated heterocyclic group containing 1, 2, 3 or 4 heteroatoms selected from O, N, or S where the optional substituent is selected from C₁₋₄alkyl, C₁₋₄alkyl substituted with 3 to 6 membered cycloalkyl, C₁₋₄alkyl substituted with optionally substituted five- or six-membered unsaturated heterocyclic group containing 1, 2, 3 or 4 heteroatoms selected from O, N, or S where the optional substituent is selected from C₁₋₄alkyl, C₁₋₄alkyl substituted with optionally substituted four- to six-membered saturated heterocyclic group containing 1 or 2 heteroatoms selected from O, N, or S where the optional substituent is selected from C₁₋₄alkyl and optionally substituted four- to six-membered saturated heterocyclic group containing 1 or 2 heteroatoms selected from O, N, or S where the optional substituent is selected from C₁₋₄alkyl;
q is selected from 0, 1 or 2; and
c is selected from 0, 1, 2 and 3;
as used in this embodiment, the term ‘cycloalkyl’ refers to a saturated monocyclic hydrocarbon ring. As used in this embodiment, the term ‘cycloalkenyl’ refers to a partially saturated monocyclic hydrocarbon ring having one or more (usually one) carbon carbon double bond(s). As used in this embodiment, the term “heterocyclyl group” shall unless the context indicates otherwise, include both aromatic and non-aromatic ring systems. As used in this embodiment, the term “heterocyclyl group” include within their scope aromatic, non-aromatic, unsaturated, partially saturated and saturated heterocyclyl ring systems, in general, unless the context indicates otherwise, such groups may be monocyclic or bicyclic (including fused, spiro and bridged bicyclic groups) As used in this embodiment, the heterocyclyl groups can be heteroaryl groups, the heterocyclyl ring can, unless the context indicates otherwise, be optionally substituted i.e. unsubstituted or substituted As used in this embodiment, the term “heteroaryl” denotes a heterocyclyl group having aromatic character, the term “heteroaryl” embraces polycyclic (e.g. bicyclic) ring systems wherein one or more rings are nonaromatic, provided that at least one ring is aromatic, and in such polycyclic systems, the group may be attached by the aromatic ring, or by a non-aromatic ring to the remainder of the compound. As used in this embodiment, the term “non-aromatic” embraces, unless the context indicates otherwise, unsaturated ring systems without aromatic character, partially saturated and saturated heterocyclyl ring systems, wherein the terms “unsaturated” and“partially saturated” refer to rings wherein the ring structure(s) contains atoms sharing more than one valence bond i.e. the ring contains at least one multiple bond e.g. a C═C, C^{∘}C or N═C bond; the heterocyclyl groups can be polycyclic fused ring systems or bridged ring systems such as the oxa- and aza analogues of bicycloalkanes, tricycloalkanes.

In embodiments the SHP2 inhibitor is a compound of having the formula (DU) immediately below, or a pharmaceutically acceptable salt or solvate thereof:

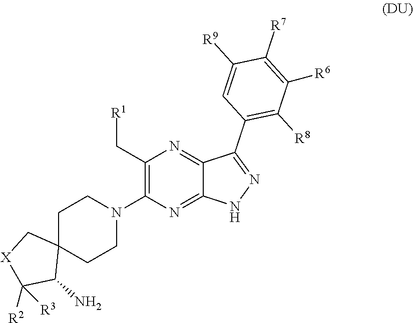

or a tautomer or a solvate or a pharmaceutically acceptable salt thereof,
wherein:
R¹ is hydrogen or hydroxyl;
R² and R³ are independently selected from hydrogen, halogen, C₁₋₄alkyl, haloC₁₋₄alkyl, hydroxyC₁₋₄alkyl and —CN;
X is O or CR⁴R⁵;
R⁴ and R⁵ are independently selected from hydrogen, halogen, hydroxyl, C₁₋₄alkyl, C₁₋₄alkoxy and haloC₁₋₄alkyl;
R⁶ and R⁷ are hydrogen, C₁₋₄alkoxy or halogen (e.g. chlorine or fluorine), or R⁶ and R⁷ join to form a Ring A which is optionally substituted by one or more (e.g. 1, 2, or 3) R¹⁰ groups;
Ring A is either:
   (i) a five-membered nitrogen-containing heterocyclic ring (e.g. an aromatic ring or a non-aromatic ring) wherein the heterocyclic ring optionally contains one or two additional heteroatoms selected from N, O and S, or
   (ii) a six-membered aromatic nitrogen-containing heterocyclic ring, wherein the heterocyclic ring optionally contains one or two additional heteroatoms selected from N, O and S; or
   (iii) a six-membered non-aromatic nitrogen-containing heterocyclic ring, wherein the heterocyclic ring optionally contains one or two additional heteroatoms selected from N and S;
R⁸ is selected from haloC₁₋₄alkyl (e.g. —CF₃), —CH₃ and halogen (e.g. chlorine or fluorine);
R⁹ is selected from hydrogen, C₁₋₄alkyl (e.g. —CH₃), haloC₁₋₄alkyl (e.g. —CF₃) and halogen (e.g. chlorine);
R¹⁰ are independently selected from halogen, cyano, cyanoC₁₋₄alkyl (e.g. —CH₂—CN), hydroxyl, ═O (oxo), C₁₋₄alkyl (e.g. —CH₃, —CH(CH₃)₂, or —CH₂CH₃), haloC₁₋₄alkyl (e.g. —CHF₂), C₁₋₄alkoxy (e.g. —OCH₃, —OCH₂CH₃ and —OCH(CH₃)₂), hydroxylC₁₋₄alkyl (e.g. —CH₂C(CH₃)₂OH, —CH(CH₃)CH₂OH, —CH(CH₃)OH, —CH₂CH₂OH or —CH₂OH), C₁₋₄alkoxyC₁₋₄alkylene (e.g. —CH₂—O—CH₃ or —CH₂—CH₂—O—CH₃), C₁₋₄alkylsulfone (e.g. —SO₂CH₃), amino, monoC₁₋₄alkylamino, diC₁₋₄alkylamino (e.g. —N(CH₃)₂), aminoC₁₋₄alkylene (e.g. —CH₂NH₂), —C₁₋₄alkylene-C(═O)NH_{(2-q)}(C₁₋₆alkyl)_{q}), —C₀₋₄alkylene-NHC(═O)C₁₋₆alkyl, sulfonamideC₀₋₄alkylene (e.g. —SO₂NR′₂ or —CH₂SO₂NR′₂
wherein R^{x} is independently selected from H and C₁₋₆alkyl), 3 to 6 membered cycloalkyl, optionally substituted five- or six-membered unsaturated heterocyclic group containing 1, 2, 3 or 4 heteroatoms selected from O, N, or S,
   where the optional substituent is selected from C₁₋₄alkyl, C₁₋₄alkyl substituted with 3 to 6 membered cycloalkyl, C₁₋₄alkyl substituted with optionally substituted five- or six-membered unsaturated heterocyclic group containing 1, 2, 3 or 4 heteroatoms selected from O, N, or S,
      where the optional substituent is selected from C₁₋₄alkyl, C₁₋₄alkyl substituted with optionally substituted four- to six-membered saturated heterocyclic group containing 1 or 2 heteroatoms selected from O, N, or S,
         where the optional substituent is selected from C₁₋₄alkyl, and optionally substituted four- to six-membered saturated heterocyclic group containing 1 or 2 heteroatoms selected from O, N, or S,
         where the optional substituent is selected from C₁₋₄alkyl; and q is selected from 0, 1 or 2;
as used in this embodiment, the term ‘cycloalkyl’ refers to a saturated monocyclic hydrocarbon ring. As used in this embodiment, the term ‘cycloalkenyl’ refers to a partially saturated monocyclic hydrocarbon ring having one or more (usually one) carbon carbon double bond(s). As used in this embodiment, the term “heterocyclyl group” shall unless the context indicates otherwise, include both aromatic and non-aromatic ring systems. As used in this embodiment, the term “heterocyclyl group” include within their scope aromatic, non-aromatic, unsaturated, partially saturated and saturated heterocyclyl ring systems, in general, unless the context indicates otherwise, such groups may be monocyclic or bicyclic (including fused, spiro and bridged bicyclic groups) As used in this embodiment, the heterocyclyl groups can be heteroaryl groups, the heterocyclyl ring can, unless the context indicates otherwise, be optionally substituted i.e. unsubstituted or substituted As used in this embodiment, the term “heteroaryl” denotes a heterocyclyl group having aromatic character, the term “heteroaryl” embraces polycyclic (e.g. bicyclic) ring systems wherein one or more rings are nonaromatic, provided that at least one ring is aromatic, and in such polycyclic systems, the group may be attached by the aromatic ring, or by a non-aromatic ring to the remainder of the compound. As used in this embodiment, the term “non-aromatic” embraces, unless the context indicates otherwise, unsaturated ring systems without aromatic character, partially saturated and saturated heterocyclyl ring systems, wherein the terms “unsaturated” and“partially saturated” refer to rings wherein the ring structure(s) contains atoms sharing more than one valence bond i.e. the ring contains at least one multiple bond e.g. a C═C, C^{∘}C or N═C bond; the heterocyclyl groups can be polycyclic fused ring systems or bridged ring systems such as the oxa- and aza analogues of bicycloalkanes, tricycloalkanes.

In an embodiment, the SHP2 inhibitor is a compound of formula (DW):

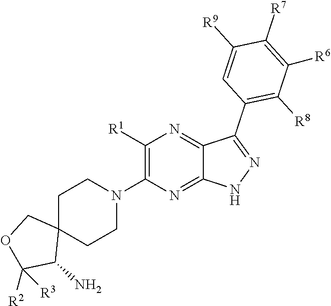

wherein R1, R2, R3, R4, R5, R6, R7, R8, and R9 are as defined in the embodiment immediately above. In an embodiment, the SHP2 inhibitor is a compound of formula (DX):

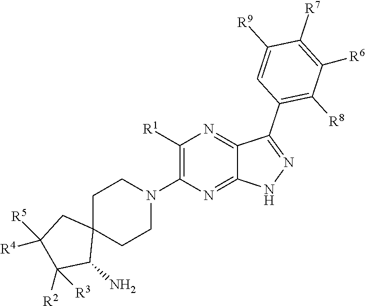

wherein R¹, R², R³, R⁴, R⁵, R⁶, R⁷, R⁸, and R⁹ are as defined as in the embodiment immediately above, which is in turn defined as in the embodiment immediately above that embodiment; as used in this embodiment, the term ‘cycloalkyl’ refers to a saturated monocyclic hydrocarbon ring. As used in this embodiment, the term ‘cycloalkenyl’ refers to a partially saturated monocyclic hydrocarbon ring having one or more (usually one) carbon carbon double bond(s). As used in this embodiment, the term “heterocyclyl group” shall unless the context indicates otherwise, include both aromatic and non-aromatic ring systems. As used in this embodiment, the term “heterocyclyl group” include within their scope aromatic, non-aromatic, unsaturated, partially saturated and saturated heterocyclyl ring systems, in general, unless the context indicates otherwise, such groups may be monocyclic or bicyclic (including fused, spiro and bridged bicyclic groups) As used in this embodiment, the heterocyclyl groups can be heteroaryl groups, the heterocyclyl ring can, unless the context indicates otherwise, be optionally substituted i.e. unsubstituted or substituted As used in this embodiment, the term “heteroaryl” denotes a heterocyclyl group having aromatic character, the term “heteroaryl” embraces polycyclic (e.g. bicyclic) ring systems wherein one or more rings are nonaromatic, provided that at least one ring is aromatic, and in such polycyclic systems, the group may be attached by the aromatic ring, or by a non-aromatic ring to the remainder of the compound. As used in this embodiment, the term “non-aromatic” embraces, unless the context indicates otherwise, unsaturated ring systems without aromatic character, partially saturated and saturated heterocyclyl ring systems, wherein the terms “unsaturated” and“partially saturated” refer to rings wherein the ring structure(s) contains atoms sharing more than one valence bond i.e. the ring contains at least one multiple bond e.g. a C═C, C^{∘}C or N═C bond; the heterocyclyl groups can be polycyclic fused ring systems or bridged ring systems such as the oxa- and aza analogues of bicycloalkanes, tricycloalkanes.

In embodiments the SHP2 inhibitor is a compound of having the formula (DY) immediately below, or a pharmaceutically acceptable salt or solvate thereof:

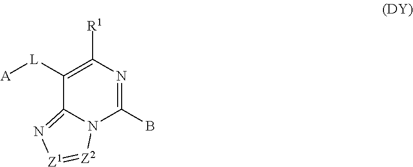

or a pharmaceutically acceptable salt thereof; wherein:
A is selected from C₆₋₁₀aryl, 5-10 membered heteroaryl, C₃₋₁₂cycloalkyl, and 4-12 membered heterocyclyl;
each A is optionally substituted with one to six R^{A} independently selected from halo, cyano, hydroxyl, azido, nitro, C₁₋₆alkyl, C₁₋₆haloalkyl, C₁₋₆alkoxyl, C₁₋₄alkylene-OH, oxo, ═NR^{a1}, —SR^{a1}, —OR^{a1}, —NR^{a1}R^{a2}, —COR^{a2}, —CONR^{a1}R^{a2}, —COOR^{a2}, —N(R^{a2})—C(O)R^{a2}, —N(R^{a2})—C(O)OR^{a2}, —N(R^{a2})—C(O)—NR^{a2}, R^{a2}, —N(R^{a2})—SO₂R^{a2}, —SO₂R^{a2}, —SO₂OR^{a2}, —SO₂NR^{a1}R^{a2}, —O—SO₂—NR^{a1}R^{a2}—O(CO)—N—R^{a1}R^{a2}, C₃₋₈cycloalkyl, 3-8 membered heterocyclyl, C₆₋₁₀aryl, 5-10 membered heteroaryl, —C₁₋₄alkylene-C₃₋₈cycloalkyl, —C₁₋₄alkylene-(3-8 membered heterocyclyl), —C₁₋₄alkylene-C₆₋₁₀aryl, and —C₁₋₄alkylene-(5-10 membered heteroaryl);
   wherein the C₃₋₈cycloalkyl, 3-8 membered heterocyclyl, C₆₋₁₀aryl, 5-10 membered heteroaryl, —C₁₋₄alkylene-C₃₋₈cycloalkyl, —C₁₋₄alkylene-(3-8 membered heterocyclyl), —C₁₋₄alkylene-C₆₋₁₀aryl, and —C₁₋₄alkylene-(5-10 membered heteroaryl) of R^{A} are independently optionally substituted with one to three groups selected from halo, cyano, hydroxyl, C₁₋₄alkyl, C₁₋₄haloalkyl, C₁₋₄alkoxyl, and C₁₋₄alkylene-OH; and
   wherein the 5-10 membered heteroaryl of A, and R^{A} contains one to five heteroatoms independently selected from S, N, and O, and optionally comprises one to three C(O) or one S(O)₂;
L is selected from a bond, —S(Oᵥ)—, —O—, —N(R^{L1})—, —C(R^{L2}R^{L3})—, —C(R^{L2}R^{L3})—C(R^{L2}R^{L3})—, —C(R^{L2})═C(R^{L2})—, and —C(O)—;
R^{L1} is selected from H, C₁₋₆alkyl, C₃₋₆cycloalkyl, 3-6 membered heterocyclyl, —C(O)—C₁₋₆alkyl, —(SO₂)—C₁₋₆alkyl, and 5-6 membered heteroaryl; wherein each C₃₋₆cycloalkyl, 3-6 membered heterocyclyl, and 5-6 membered heteroaryl of R^{L} is optionally substituted with one to three groups selected from halo, C₁₋₄alkyl, and C₁₋₄haloalkyl; and
R^{L2} and R^{L3} are independently selected from H, halo, hydroxyl, C₁₋₄alkyl, C₁₋₄haloalkyl, C₁₋₄alkylene-OH, and C₃₋₆cycloalkyl; wherein each C₁₋₄alkylene-OH, and C₃₋₆cycloalkyl of R² and R^{L3} is optionally substituted with one to three halo; or
R^{L2} and R^{L3} together with the atom to which they are attached form a 3-6 membered cycloalkyl or heterocyclyl; wherein the 3-6 membered cycloalkyl or heterocyclyl is optionally substituted with one to three groups selected from halo, hydroxyl, C₁₋₄alkoxyl, —(SO₂)— C₁₋₆alkyl, oxo, and nitro;
Z¹ and Z² are independently selected from N and CR³; wherein R³ is selected from H, halo, hydroxyl, cyano, C₁₋₄alkyl, C₁₋₄haloalkyl, C₁₋₄alkoxyl, C₁₋₄alkylene-OH, —NR^{c1}R^{C2}, —C(O)OR^{c1}, C₆₋₁₀aryl, and 5-10 membered heteroaryl;
   wherein each C₆₋₁₀aryl, and 5-10 membered heteroaryl of R³ is independently optionally substituted with one to three groups independently selected from halo, hydroxyl, cyano, C₁₋₄alkyl, C₁₋₄haloalkyl, C₁₋₄alkoxyl, —N(R^{c1})—SO₂R^{c1}, and —SO₂R^{c1};
R¹ is selected from H, halo, —NR^{c1}R^{C2}, C₁₋₄alkyl, and C₁₋₄haloalkyl;
B is selected from

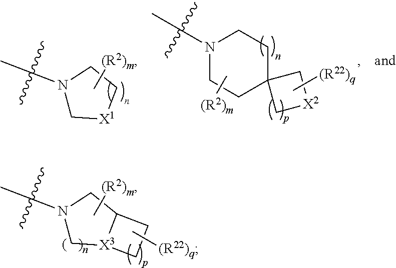

X¹ is selected from —CR²²—, —CHR², —CH₂—, —O—, —NR²—, and —S(Oᵥ)—;
X² is selected from —CR²²₂—, —CHR²²—, —CH₂—, —O—, —NH—, —NR²²—, —CO—, and —S(O_{y})—;
X³ is selected from CH and N;
each R² is independently selected from halo, cyano, nitro, —O—C₁₋₆alkyl, oxo, —NR^{c1}R^{C2}, —(SOv)-R^{c1}, —NR^{c1}(SOᵥR^{c1}, —C(O)OR^{c1}, —C(O)—NR^{c1}R^{C2}, —S(O₂)—NR^{c1}R^{C2}, C₁₋₆alkyl, C₂₋₆alkenyl, C₂₋₆alkynyl, C₁₋₄alkylene-OH, —C₁₋₄alkylene-NR^{c1}R^{C2}, C₃₋₈cycloalkyl, 3-6 membered heterocyclyl, —O—C₃₋₆cycloalkyl, —O-(3-6 membered heterocyclyl), C₆₋₁₀aryl, and 5-10 membered heteroaryl;
   wherein the C₁₋₆alkyl, C₂₋₆alkenyl, C₂₋₆alkynyl, C₃₋₈cycloalkyl, 3-6 membered heterocyclyl, —O—C₁₋₆alkyl, —O—C₃₋₈cycloalkyl, —O-(3-6 membered heterocyclyl), C₆₋₁₀aryl, and 5-10 membered heteroaryl of R² is optionally substituted with one to three groups independently selected from halo, —NR^{a1}R^{a2}, hydroxyl, azido, cyano, —SH, C₁₋₄alkyl, C₁₋₄haloalkyl, C₁₋₄alkoxyl, C₁₋₄alkylene-OH, —C(O)OR^{a1}, C₃₋₈cycloalkyl, 3-6 membered heterocyclyl, C₆₋₁₀aryl, and 5-10 membered heteroaryl; or
two R², together with the atoms to which they are attached form a spiro, fused or bridged 3-12 membered cycloalkyl or heterocyclyl; wherein the spiro, fused or bridged 3-12 membered cycloalkyl or heterocyclyl is optionally substituted with one to three groups selected from halo, —NR^{a1}R^{a2}, hydroxyl, azido, cyano, —SH, C₁₋₄alkyl, C₁₋₄haloalkyl, C₁₋₄alkoxyl, and C₁₋₄alkylene-OH;
each R²² is independently selected from halo, —NR^{c1}R^{C2}, hydroxyl, azido, cyano, oxo, —C(O)OR^{c1}, C₁₋₄alkyl, C₁₋₄haloalkyl, C₁₋₄alkoxyl, C₁₋₄alkylene-NR^{c1}R^{C2}, —C₁₋₄alkylene-O—C₁₋₄alkyl, —C₁₋₄alkylene-OH, —COR^{c1}, —CO—NR^{c1}R^{C2}, —C(O)OR^{c1}, —N(R^{c1})—C(O)R^{c1}, —N(R^{c1})—C(O)OR^{c}, —N(R^{c1})—C(O)—NR^{c1}R^{C2}, —N(R^{c1})—(SOᵥ)R^{c1}, —SO₂R^{c1}, —SO₂OR^{c1}, —SO₂R^{c1}R^{C2}, C₃₋₈cycloalkyl, 3-6 membered heterocyclyl, C₆₋₁₀aryl, and 5-10 membered heteroaryl; or
two R²², together with the atoms to which they are attached, form a 3-12 membered spiro, bridged or fused ring E;
   wherein the ring E is selected from cycloalkyl, heterocyclyl, aryl, and heteroaryl; and
   wherein the ring E is optionally substituted with one to three groups selected from halo, cyano, hydroxyl, azido, nitro, C₁₋₆alkyl, C₁₋₆haloalkyl, C₁₋₆alkoxyl, C₁₋₄alkylene-OH, oxo, ═NR^{a1}, —SR^{a1}, —OR^{a1}, —NR^{a1}R^{a2}, —COR^{a2}, —CONR^{a1}R^{a2}, —COOR^{a2}, —N(R^{a2})—C(O)R^{a2}, —N(R^{a2})—C(O)OR^{a2}, —N(R^{a2})—C(O)—NR^{a2}R^{a2}, —N(R^{a2})—SO₂R^{a2}, —SO₂R^{a2}, —SO₂OR^{a2}, —SO₂NR^{a1}R^{a2}, —O—SO₂—NR^{a1}R^{a2}, —O(CO)—NR^{a1}R^{a2}, C₃₋₈cycloalkyl, 3-8 membered heterocyclyl, C₆₋₁₀aryl, 5-10 membered heteroaryl, —C₁₋₄alkylene-C₃₋₈cycloalkyl, —C₁₋₄alkylene-(3-8 membered heterocyclyl), —C₁₋₄alkylene-C₆₋₁₀aryl, and —C₁₋₄alkylene-(5-10 membered heteroaryl);
   R^{a1} is selected from H, C₁₋₄alkyl, C₁₋₄haloalkyl, C₁₋₄alkylene-OH, C₁₋₄alkylene-COOR^{a2}, —C₁₋₄alkylene-C₁₋₄alkoxyl, and —C(O)—NH₂;
   R^{a2} is selected from H, C₁₋₄alkyl, C₁₋₄haloalkyl, C₁₋₄alkylene-OH, C₃₋₈cycloalkyl, 3-8 membered heterocyclyl, C₆₋₁₀aryl, and 5-10 membered heteroaryl;
   wherein the C₁₋₄alkyl, C₁₋₄haloalkyl, C₁₋₄alkylene-OH, C₃₋₈cycloalkyl, 3-8 membered heterocyclyl, C₆₋₁₀aryl, and 5-6 membered heteroaryl of R^{a2} are independently optionally substituted with one to three groups selected from halo, cyano, hydroxyl, —COOR^{a3}, C₁₋₄alkyl, C₁₋₄haloalkyl, C₁₋₄alkylene-OH, and C₁₋₄alkoxyl;
wherein R^{a3} is selected from H, C₁₋₄alkyl, and C₁₋₄haloalkyl;
R^{c1} and R^{C2} are independently selected from H, C₁₋₆alkyl, and C₁₋₆haloalkyl; wherein each of the C₁₋₆alkyl and C₁₋₆haloalkyl of R^{c1} and R^{C2} is optionally substituted with one or two groups selected from C₁₋₄alkoxyl, and C₁₋₄alklene-OH;
v is selected from 0, 1, and 2;
n is selected from 0, 1, 2, 3, and 4;
m is selected from 0, 1, 2, 3, 4, 5, 6, 7, and 8;
q is selected from 0, 1, 2, 3, and 4; and
p is selected from 0, 1, 2, 3, and 4;
as used in this embodiment, “Aryl” refers to an aromatic carbocyclic group having a single ring (e.g. monocyclic) or multiple rings (e.g. bicyclic or tricyclic) including fused systems. As used in this embodiment, Aryl, however, does not encompass or overlap in any way with heteroaryl defined in this paragraph. If one or more aryl groups are fused with a heteroaryl ring, the resulting ring system of this paragraph is heteroaryl. As used in this embodiment, “Cycloalkyl” refers to a saturated or partially saturated cyclic alkyl group having a single ring or multiple rings including fused, bridged, and spiro ring systems. As used in this embodiment, the term “cycloalkyl” includes cycloalkenyl groups (i.e. the cyclic group having at least one double bond). As used in this embodiment, cycloalkyl groups also include partially unsaturated ring systems containing one or more double bonds, including fused ring systems with one aromatic ring and one non-aromatic ring, but not fully aromatic ring systems. As used in this embodiment, “Bridged” refers to a ring fusion wherein non-adjacent atoms on a ring are joined by a divalent substituent. As used in this embodiment, the term “fused” refers to a ring which is bound to an adjacent ring. As used in this embodiment, “Spiro” refers to a ring substituent which is joined by two bonds at the same atom. As used in this embodiment, “Heteroalkyl” refers to an alkyl group in which one or more of the carbon atoms (and any associated hydrogen atoms) are each independently replaced with the same or different heteroatomic group. As used in this embodiment, the term “heteroalkyl” includes unbranched or branched saturated chain having carbon and heteroatoms. As used in this embodiment, “Heteroaryl” refers to an aromatic group having a single ring, multiple rings, or multiple fused rings, with one or more ring heteroatoms independently selected from nitrogen, oxygen, and sulfur. As used in this embodiment, heteroaryl also includes oxidized forms of a heteroaryl as defined herein. As used in this embodiment, for example, heteroaryl includes a pyridyl and any oxidized form of pyridyl such as 2-pyridone, 4-pyridone, or pyridine N-oxide. As used in this embodiment, the term “heterocyclyl” or“heterocycle” refers to a single saturated or partially unsaturated non-aromatic ring or a non-aromatic multiple ring system that has at least one heteroatom in the ring (i.e., at least one annular heteroatom selected from oxygen, nitrogen, and sulfur). As used in this embodiment, the rings of the multiple condensed ring (e.g. bicyclic heterocyclyl) system can be connected to each other via fused, spiro and bridged bonds when allowed by valency requirements. As used in this embodiment, the term “heterocyclyl” or“heterocyclic ring” or“heterocycle” includes heterocycloalkenyl groups (i.e., the heterocyclyl group having at least one double bond). As used in this embodiment, heterocyclyl may be a single ring or multiple rings wherein the multiple rings may be fused, bridged, or spiro. As used in this embodiment, the term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used in this embodiment, “bridged-heterocyclyl” includes bicyclic and tricyclic ring systems. As used in this embodiment, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. As used in this embodiment, the terms“heterocycle”, “heterocyclyl”, and“heterocyclic ring” are used interchangeably.

In embodiments the SHP2 inhibitor is a compound of having the formula (DZ) immediately below, or a pharmaceutically acceptable salt or solvate thereof:

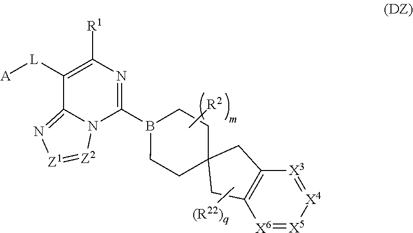

wherein A, L, Z¹, Z², R¹, R^{a1}, R^{a2}, R², R²², m, p, and q are as defined in in the embodiment immediately above. Each X³, X⁴, X⁵, and X⁶ is independently selected from CR^{xx}, and N; R^{xx} is selected from H, halo, cyano, hydroxyl, azido, nitro, C₁₋₆alkyl, C₁₋₆haloalkyl, C₁₋₆alkoxyl, C₁₋₄alkylene-OH, —SR^{a1}, —OR^{a1}, —NR^{a1}R^{a2}, —COR^{a2}, —CONR^{a1}R^{a2}, —COOR^{a2}, —N(R^{a2})—C(O)R^{a2}, —N(R^{a2})—C(O)OR^{a2}, —N(R^{a2})—C(O)—NR^{a2}R^{a2}, —N(R^{a2})—SO₂R₂, —SO₂R^{a2}, —SO₂OR^{a2}, —SO₂NR^{a1}R^{a2}, —O—SO₂—NR^{a1}R^{a2}, —O(CO)—NR^{a1}R^{a2}, C₃₋₈cycloalkyl, 3-8 membered heterocyclyl, C₆₋₁₀aryl, 5-10 membered heteroaryl, —C₁₋₄ alkylene-C₃₋₆cycloalkyl, —C₁₋₄alkylene-(3-8 membered heterocyclyl), —C₁₋₄alkylene-C₆₋₁₀aryl, and —C₁₋₄alkylene-(5-10 membered heteroaryl);
as used in this embodiment, “Aryl” refers to an aromatic carbocyclic group having a single ring (e.g. monocyclic) or multiple rings (e.g. bicyclic or tricyclic) including fused systems. As used in this embodiment, Aryl, however, does not encompass or overlap in any way with heteroaryl defined in this paragraph. If one or more aryl groups are fused with a heteroaryl ring, the resulting ring system of this paragraph is heteroaryl. As used in this embodiment, “Cycloalkyl” refers to a saturated or partially saturated cyclic alkyl group having a single ring or multiple rings including fused, bridged, and spiro ring systems. As used in this embodiment, the term “cycloalkyl” includes cycloalkenyl groups (i.e. the cyclic group having at least one double bond). As used in this embodiment, cycloalkyl groups also include partially unsaturated ring systems containing one or more double bonds, including fused ring systems with one aromatic ring and one non-aromatic ring, but not fully aromatic ring systems. As used in this embodiment, “Bridged” refers to a ring fusion wherein non-adjacent atoms on a ring are joined by a divalent substituent. As used in this embodiment, the term “fused” refers to a ring which is bound to an adjacent ring. As used in this embodiment, “Spiro” refers to a ring substituent which is joined by two bonds at the same atom. As used in this embodiment, “Heteroalkyl” refers to an alkyl group in which one or more of the carbon atoms (and any associated hydrogen atoms) are each independently replaced with the same or different heteroatomic group. As used in this embodiment, the term “heteroalkyl” includes unbranched or branched saturated chain having carbon and heteroatoms. As used in this embodiment, “Heteroaryl” refers to an aromatic group having a single ring, multiple rings, or multiple fused rings, with one or more ring heteroatoms independently selected from nitrogen, oxygen, and sulfur. As used in this embodiment, heteroaryl also includes oxidized forms of a heteroaryl as defined herein. As used in this embodiment, for example, heteroaryl includes a pyridyl and any oxidized form of pyridyl such as 2-pyridone, 4-pyridone, or pyridine N-oxide. As used in this embodiment, the term “heterocyclyl” or “heterocycle” refers to a single saturated or partially unsaturated non-aromatic ring or a non-aromatic multiple ring system that has at least one heteroatom in the ring (i.e., at least one annular heteroatom selected from oxygen, nitrogen, and sulfur). As used in this embodiment, the rings of the multiple condensed ring (e.g. bicyclic heterocyclyl) system can be connected to each other via fused, spiro and bridged bonds when allowed by valency requirements. As used in this embodiment, the term “heterocyclyl” or “heterocyclic ring” or“heterocycle” includes heterocycloalkenyl groups (i.e., the heterocyclyl group having at least one double bond). As used in this embodiment, heterocyclyl may be a single ring or multiple rings wherein the multiple rings may be fused, bridged, or spiro. As used in this embodiment, the term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used in this embodiment, “bridged-heterocyclyl” includes bicyclic and tricyclic ring systems. As used in this embodiment, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. As used in this embodiment, the terms “heterocycle”, “heterocyclyl”, and“heterocyclic ring” are used interchangeably.

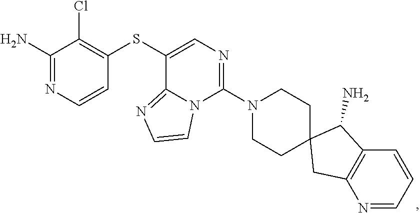 [SMILES: Nc1nccc(Sc2cnc(N3CCC4(CC3)Cc3ncccc3[C@H]4N)n3ccnc23)c1Cl]

In embodiments, the compound is, or a pharmaceutically acceptable salt or solvate

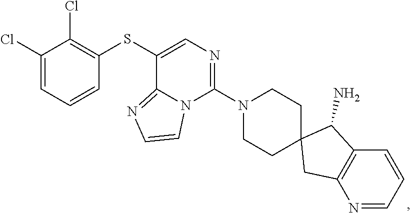 [SMILES: N[C@@H]1c2cccnc2CC12CCN(c1ncc(Sc3cccc(Cl)c3Cl)c3nccn13)CC2]

thereof. In embodiments, the compound is N, or a pharmaceutically acceptable salt or solvate thereof.

In some embodiments, the compositions and methods described herein may utilize one or more SHP2 inhibitor selected from, but not limited to, any SHP2 inhibitor disclosed in Chen, Ying-Nan P et al, 148 Nature Vol 535 7 Jul. 2016, incorporated herein by reference in its entirety, including SHP099, disclosed therein. The compositions and methods described herein may utilize one or more SHP2 inhibitor selected from, but not limited to any SHP2 inhibitor disclosed in any one of PCT applications PCT/US2017/041577 (WO2018013597); PCT/US2018/013018 (WO2018136264); and PCT/US2018/013023 (WO2018136265), each of which is incorporated herein by reference in its entirety. The compositions and methods described herein may utilize one or more SHP2 inhibitor selected from, but not limited to any SHP2 inhibitor disclosed in PCT applications PCT/IB2015/050343 (WO2015107493); PCT/IB2015/050344 (WO2015107494); PCT/IB2015/050345 (WO201507495); PCT/IB2016/053548 (WO2016/203404); PCT/IB2016/053549 (WO2016203405); PCT/IB2016/053550 (WO2016203406); PCT/US2010/045817 (WO2011022440); PCT/US2017/021784 (WO2017156397); PCT/US2016/060787 (WO2017079723); and PCT/CN2017/087471 (WO 2017211303), each of which is incorporated herein by reference in its entirety. The compositions and methods described herein may utilize one or more SHP2 inhibitor selected from, but not limited to any SHP2 inhibitor disclosed in Chen L, et al., Mol Pharmacol. 2006 August; 70(2):562-70, incorporated herein by reference in its entirety, including NSC-87877 disclosed therein. The compositions and methods described herein may utilize TN0155, described under ClinicalTrials.gov Identifier: NCT03114319, available at world wide web address: clinicaltrials.gov/ct2/show/NCT03114319, incorporated herein by reference in its entirety. The compositions and methods described herein may utilize one or more SHP2 inhibitor selected from, but not limited to any SHP2 inhibitor disclosed in applications WO2017210134, WO2019213318, WO2019167000, WO2021033153, WO2020177653, WO2020061103, WO2020061101, U.S. Ser. No. 10/894,797, WO2020072656, U.S. Ser. No. 16/691,092, WO2020076723, WO2018057884, WO2017211303, and WO2015107495; each of which is incorporated herein by reference in its entirety. The compositions and methods described herein may utilize one or more SOS inhibitor selected from, but not limited to any SOS inhibitor disclosed in PCT applications WO2021074227, WO2019201848, WO2018172250, WO2019122129, and WO2018115380; each of which is incorporated herein by reference in its entirety.

A composition comprising: an inhibitor against (a) Kras G12D; and an inhibitor against (b) Kras G12C. In embodiments, the composition further comprises an inhibitor against (c) one or more signaling molecules selected from Table 1. In embodiments the composition includes an inhibitor of KRAS G12C described in WO17/201161, WO19/099524, WO20/101736, WO20/047192, WO19/217307, WO20/146613, PCT/US2020/040254, WO20/055755, WO20/055758, WO20/055760, WO20/055756, WO20/055761, WO20/118066, WO21/061749, WO21/041671, or PCT/US2021/019678, all of which are herein incorporated by reference in their entirety for all uses. In embodiments the composition includes an inhibitor of KRAS G12C described in WO2018/119183, WO2018/217651, WO2019/051291, WO2019/217691, WO2019/241157, WO2019/213526, WO2019/213516, WO2018/143315, WO2020/027083, WO2020/027084, WO2018/206539, WO2019/110751, WO2019/215203, WO2019/155399, WO2019/150305, WO2020/081282, U.S. Pat. No. 10,968,214, WO2020/035031, WO2020/212895, CN111773225A, WO2014/206343, wo2016/165626, WO2018/007885, WO2021/058018, WO2021/055728, WO2021/057832, WO2021/052499, WO2021/043322, WO2021/037018, WO2021/031952, WO2020/050890, WO2020/106640, WO2017/080979, WO2020/178282, WO2019/141250, WO2020/259432, WO2016/176338, WO2015/184349, WO2020/163594, WO2020/163598, WO2020/132071, WO2020097537, WO2020177629, WO2020221239, WO2021023247, WO2020259573A1, WO2021027943. WO2021000885, WO2013177983, US20170000800, CN103450204, WO2020239123, WO2021081212; WO2021083167, WO2021084765, WO2021085653, or WO2021086833 all of which are herein incorporated by reference in their entirety for all uses. In an aspect is provided a method of inhibiting cell proliferation signaling in a cell, comprising: downregulating, in the cell, expression or activity of KRAS (e.g., Kras G12D, Kras G12C, wildtype KRas, a mutant Kras). In embodiments the composition includes an inhibitor of KRAS G12C described in WO2021083167, WO2021084765, WO2021085653, WO2021086833, U.S. application Ser. No. 17/088,986 corresponding to US publication US2021/0130369; all of which are herein incorporated by reference in their entirety for all uses.

In an aspect is provided a method of treating a proliferative disorder in a subject, comprising administering an effective amount of an inhibitor of Kras G12D and an inhibitor of Kras G12C to said patient. In embodiments, the method further comprises administering an inhibitor of against one or more signaling molecules selected from Table 1.

In an aspect is provided a composition comprising an inhibitor of Kras G12D, an inhibitor of Kras G12C, and one or more inhibitors against one or more signaling molecules selected from Table 1.

In an aspect is provided a composition comprising an inhibitor of Kras G12D and one or more inhibitors against one or more signaling molecules selected from Table 1.

In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of SHP2 selected from

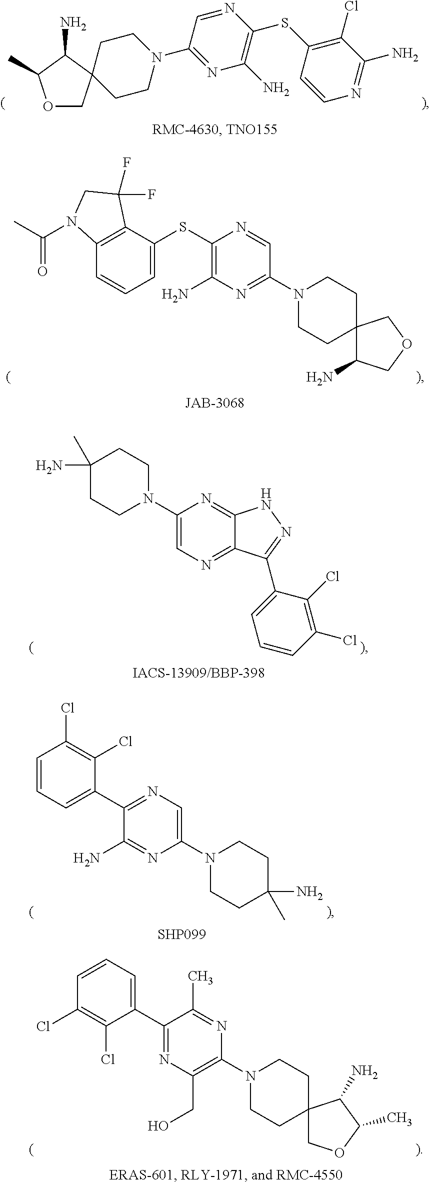 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of SOS selected from

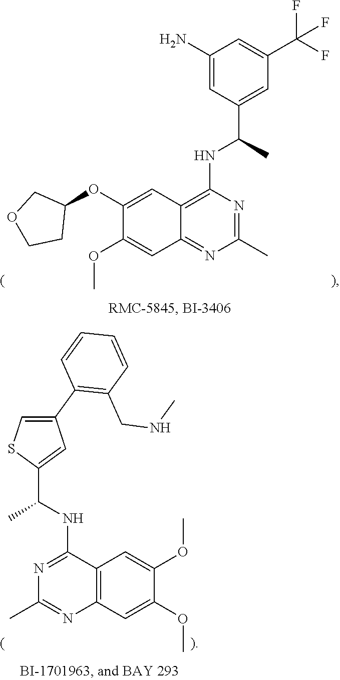

In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of EGFR selected from afatinib, erlotinib, gefitinib, lapatinib, cetuximab panitumumab, osimertinib, olmutinib, and EGF-816. In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has a formula selected from formulae CA to CE and CF′ to CJ′ and any embodiments thereof, as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CA), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CA), as disclosed herein, and the inhibitor of one or more signaling molecules selected from Table 1 is an inhibitor of SHP2 selected from

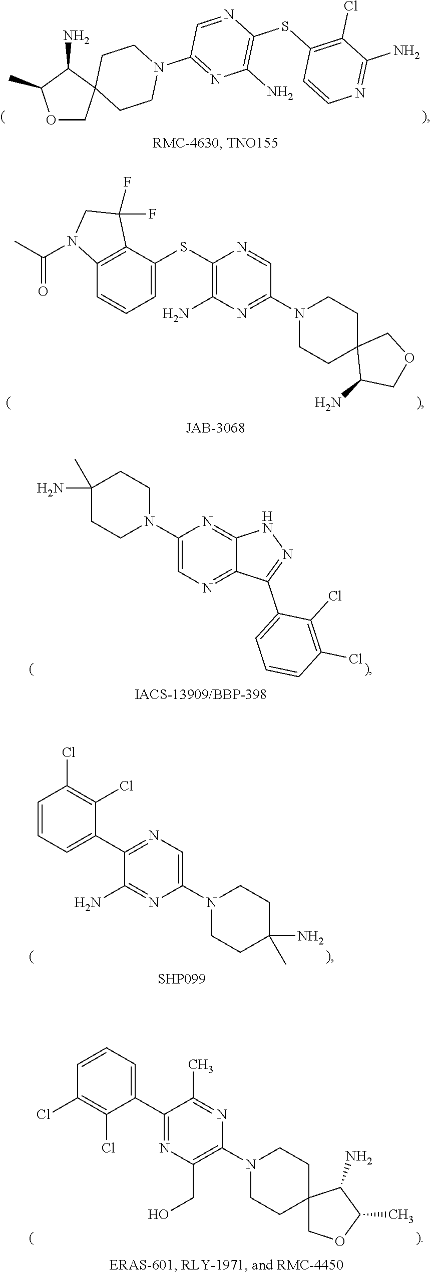 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CA), and the inhibitor of one or more signaling molecules selected from Table 1 is an inhibitor of SOS selected from

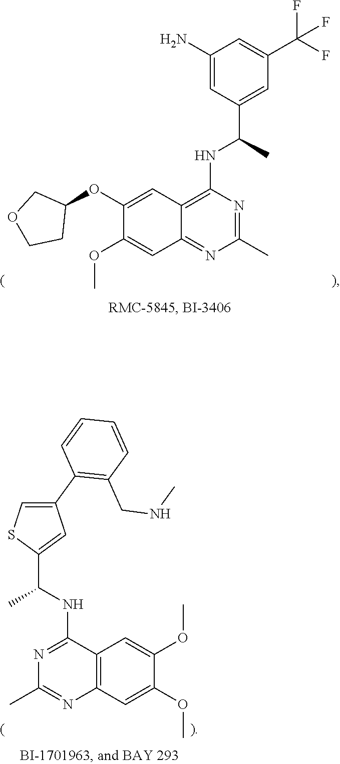

In embodiments of the subject composition, the Kras G12D inhibitor has the formula (CA), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olmutinib, and EGF-816. In embodiments of the subject composition, the Kras G12D inhibitor has the formula (CA), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CA), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CA), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CA), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CB), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CB), as disclosed herein, and the inhibitor of one or more signaling molecules selected from Table 1 is an inhibitor of SHP2 selected from

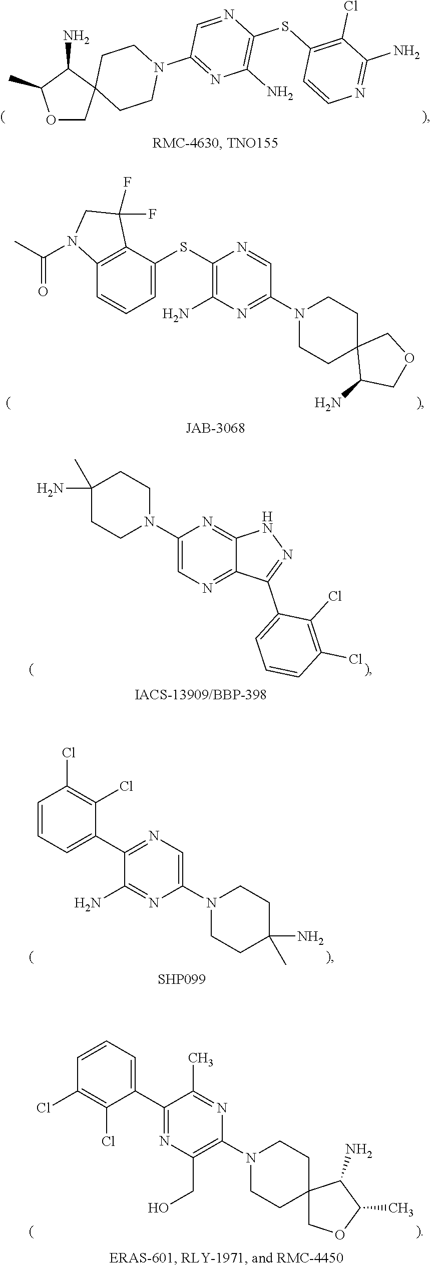 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (GB), and the inhibitor of one or more signaling molecules selected from Table 1 is an inhibitor of SOS selected from

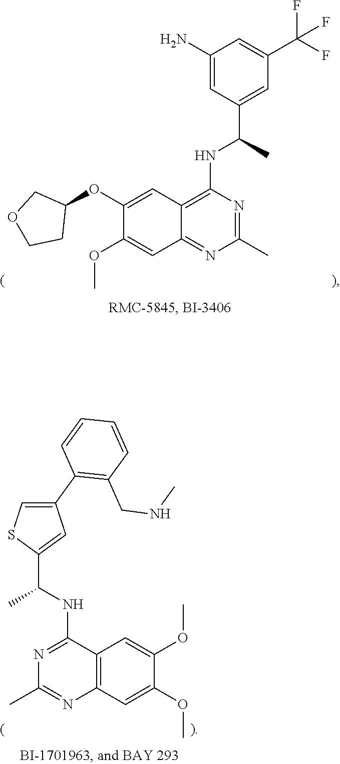

In embodiments of the subject composition, the Kras G12D inhibitor has the formula (GB), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CB), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CB), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CB), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CB), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CC), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CC), as disclosed herein, and the inhibitor of one or more signaling molecules selected from Table 1 is an inhibitor of SHP2 selected from

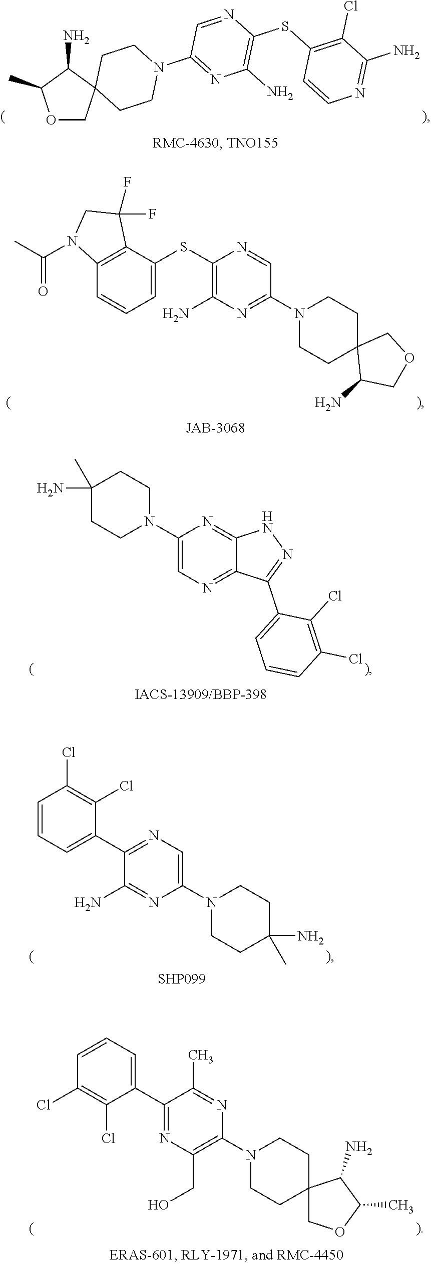 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CC), and the inhibitor of one or more signaling molecules selected from Table 1 is an inhibitor of SOS selected from

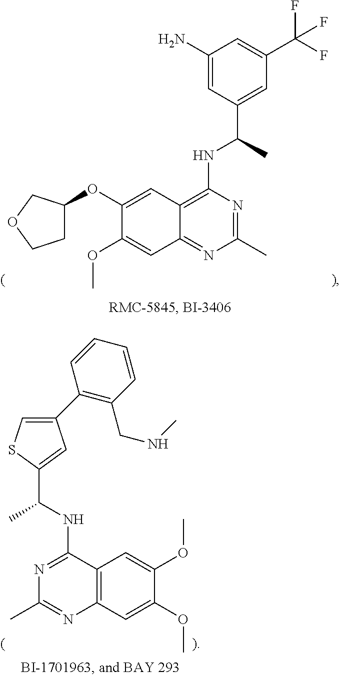 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1 ; "BI-1701963, and BAY 293"]

In embodiments of the subject composition, the Kras G12D inhibitor has the formula (CC), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CC), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CC), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CC), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CC), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CD), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CD), as disclosed herein, and the inhibitor of one or more signaling molecules selected from Table 1 is an inhibitor of SHP2 selected from

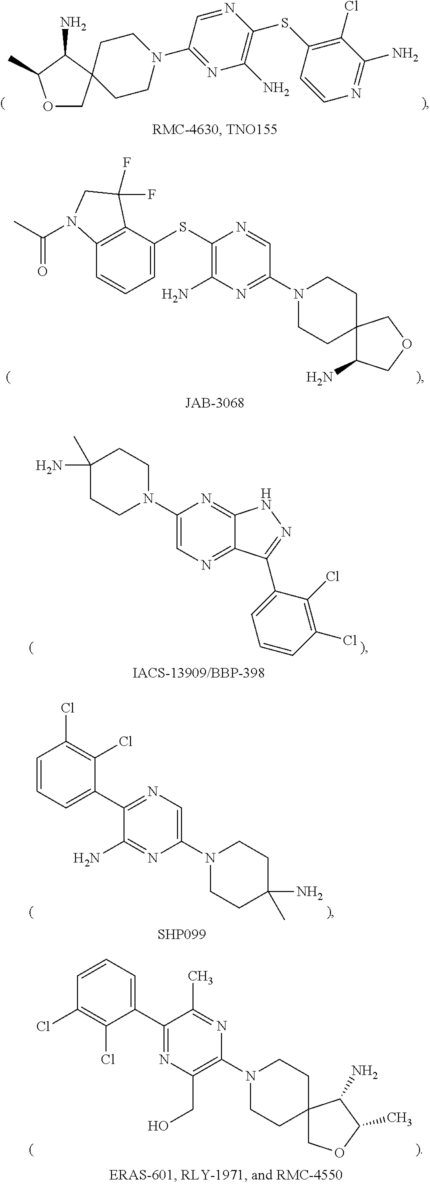 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CD), and the inhibitor of one or more signaling molecules selected from Table 1 is an inhibitor of SOS selected from

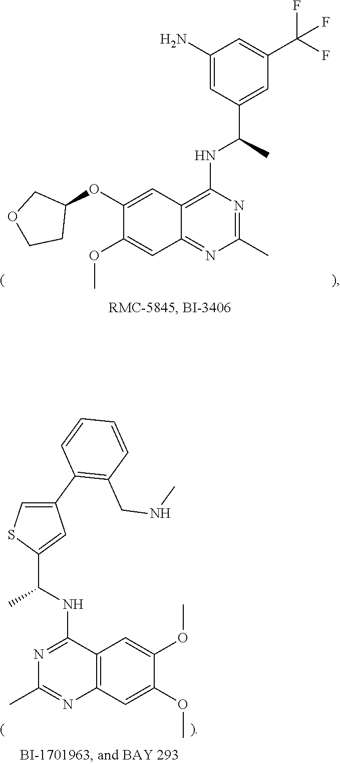

In embodiments of the subject composition, the Kras G12D inhibitor has the formula (CD), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CD), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CD), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CD), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CD), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CE), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CE), as disclosed herein, and the inhibitor of one or more signaling molecules selected from Table 1 is an inhibitor of SHP2 selected from

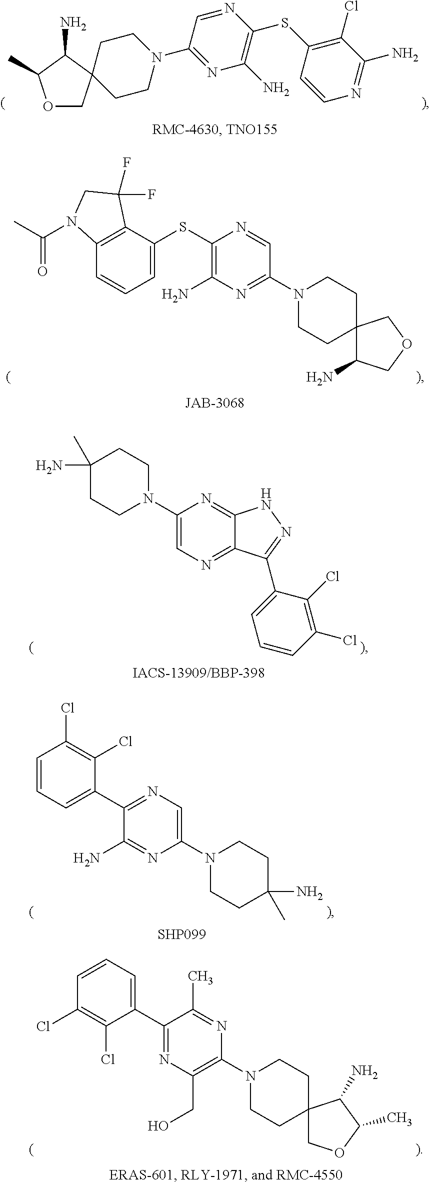 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CE), and the inhibitor of one or more signaling molecules selected from Table 1 is an inhibitor of SOS selected from

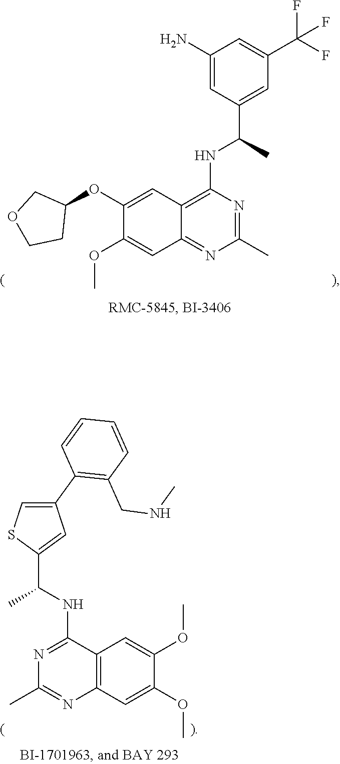

In embodiments of the subject composition, the Kras G12D inhibitor has the formula (CE), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CE), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CE), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CE), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CE), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CF′), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CF′), as disclosed herein, and the inhibitor of one or more signaling molecules selected from Table 1 is an inhibitor of SHP2 selected from

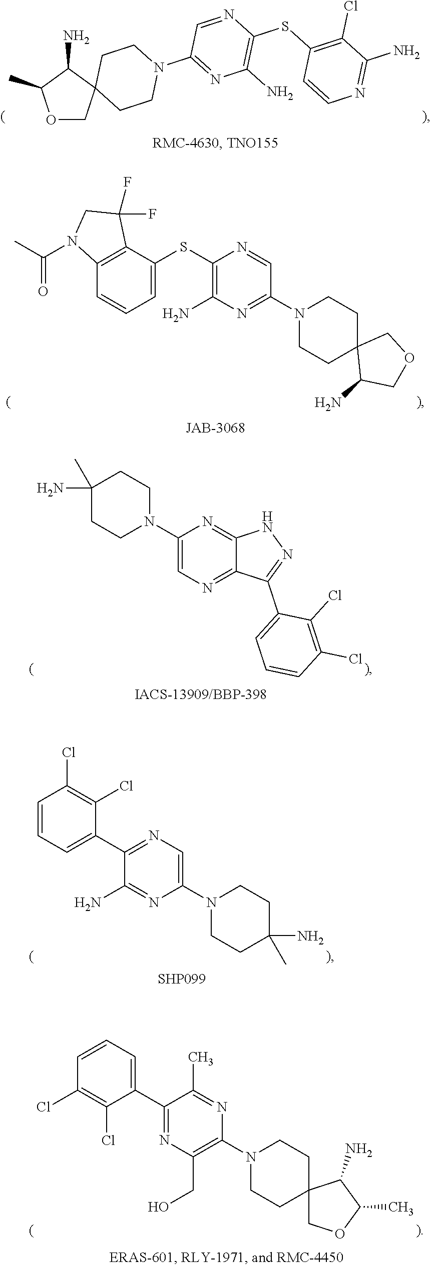 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CF′), and the inhibitor of one or more signaling molecules selected from Table 1 is an inhibitor of SOS selected from

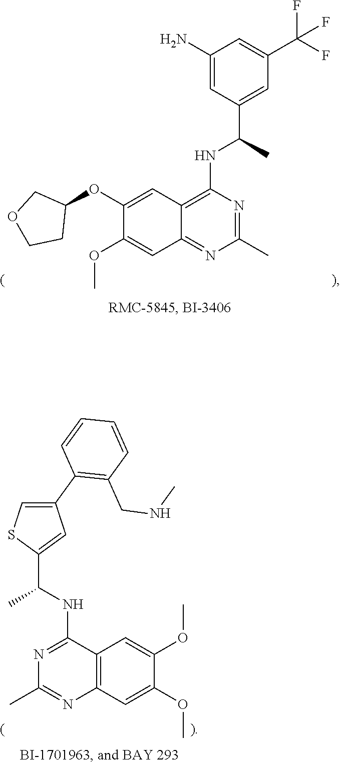

In embodiments of the subject composition, the Kras G12D inhibitor has the formula (CF′), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CF′), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CF′), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CF′), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CF′), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CG′), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CG′), as disclosed herein, and the inhibitor of one or more signaling molecules selected from Table 1 is an inhibitor of SHP2 selected from

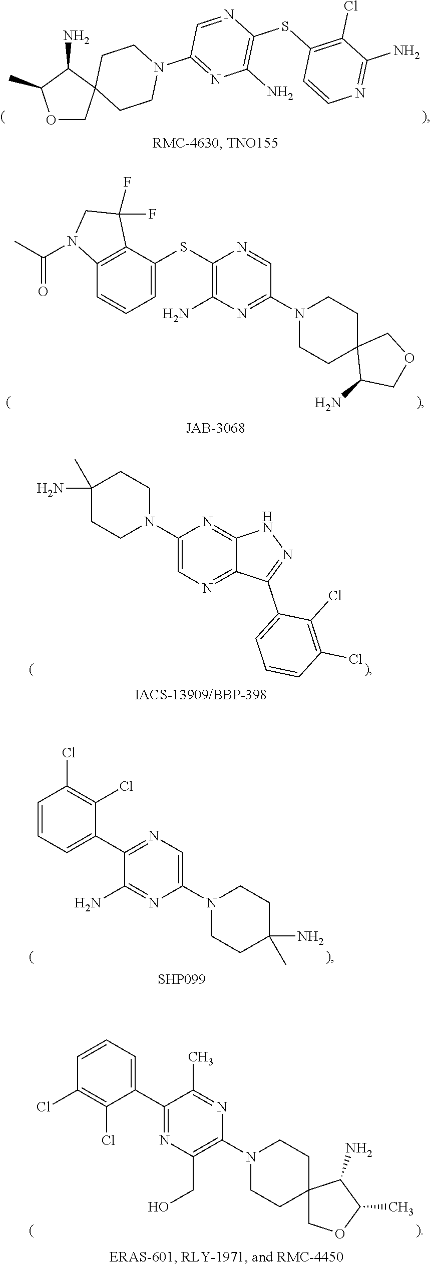 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CG′), and the inhibitor of one or more signaling molecules selected from Table 1 is an inhibitor of SOS selected from

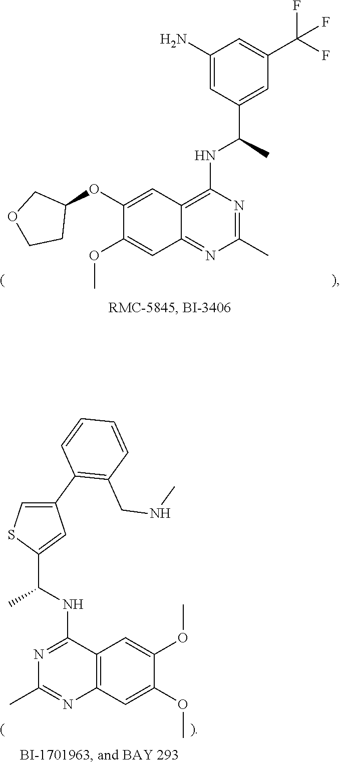

In embodiments of the subject composition, the Kras G12D inhibitor has the formula (CG′), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olmutinib, and EGF-816. In embodiments of the subject composition, the Kras G12D inhibitor has the formula (CG′), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CG′), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CG′), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CG′), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CH′), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CH′), as disclosed herein, and the inhibitor of one or more signaling molecules selected from Table 1 is an inhibitor of SHP2 selected from

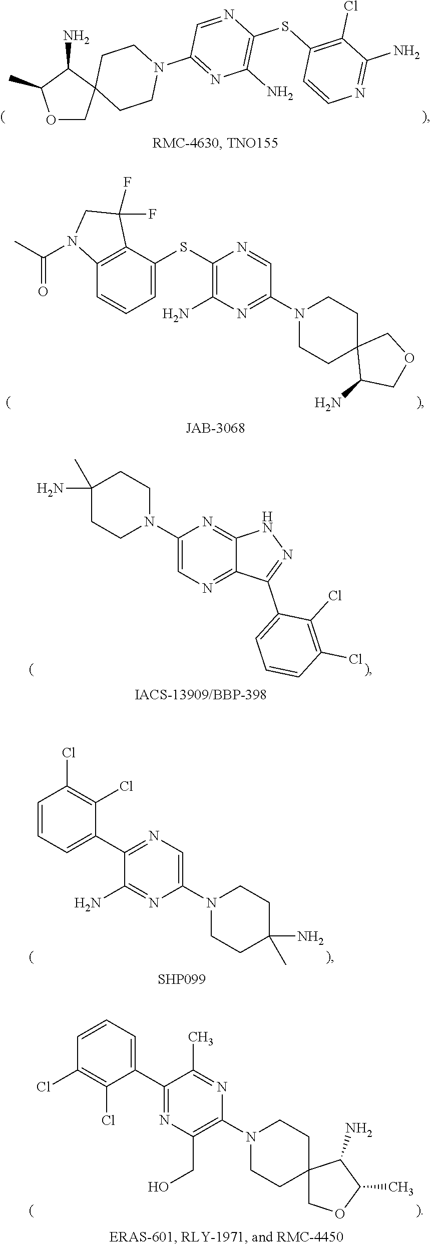 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CH′), and the inhibitor of one or more signaling molecules selected from Table 1 is an inhibitor of SOS selected from

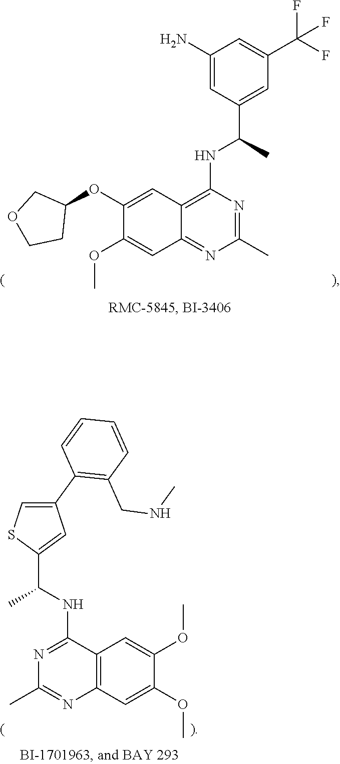

In embodiments of the subject composition, the Kras G12D inhibitor has the formula (CH′), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CH′), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CH′), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CH′), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CH′), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CI′), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CI′), as disclosed herein, and the inhibitor of one or more signaling molecules selected from Table 1 is an inhibitor of SHP2 selected from

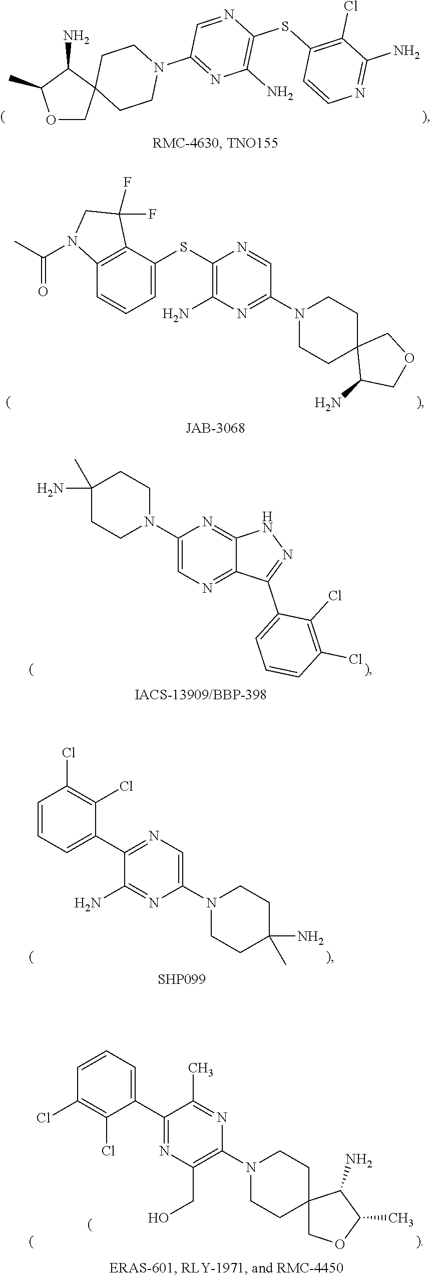 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CI′), and the inhibitor of one or more signaling molecules selected from Table 1 is an inhibitor of SOS selected from

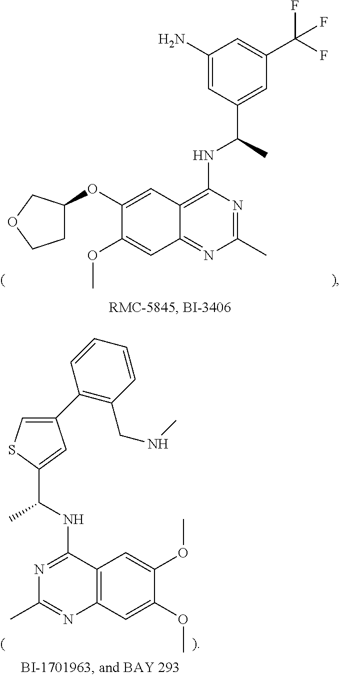 [SMILES: COc1cc2nc(C)nc(N[C@H](C)c3cc(N)cc(C(F)(F)F)c3)c2cc1O[C@H]1CCOC1 ; CNCc1ccccc1-c1csc([C@@H](C)Nc2nc(C)nc3cc(OC)c(OC)cc23)c1 ; "BI-1701963, and BAY 293"]

In embodiments of the subject composition, the Kras G12D inhibitor has the formula (CI′), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CI′), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CI′), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CI′), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CI′), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula (CJ′), as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula (CJ′), as disclosed herein, and the inhibitor of one or more signaling molecules selected from Table 1 is an inhibitor of SHP2 selected from

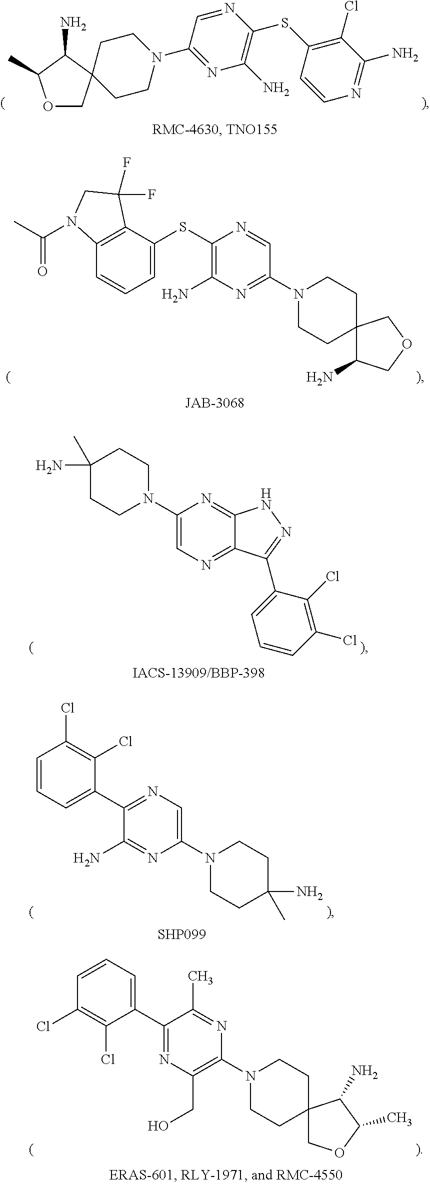 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula (CJ′), and the inhibitor of one or more signaling molecules selected from Table 1 is an inhibitor of SOS selected from

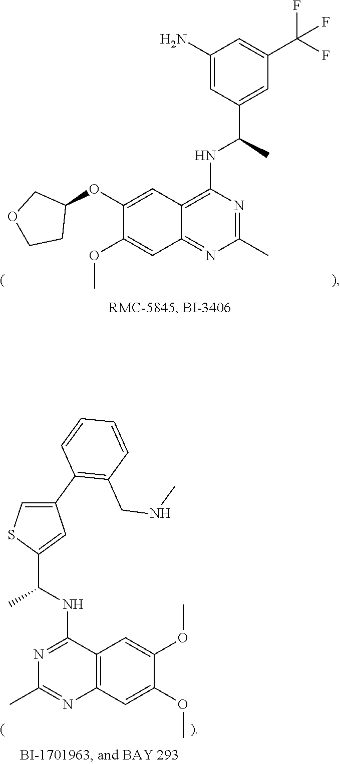

In embodiments of the subject composition, the Kras G12D inhibitor has the formula (CJ′), as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula (CJ′), as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula (CJ′), as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula (CJ′), as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula (CJ′), as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has the formula selected from CF′ to CJ′ and any embodiments thereof, as disclosed herein, and the inhibitor of one or more signaling molecules selected from Table 1 has a formula selected from formulae CF to CZ, CK′, DA to DZ, A to F, N to Z, Z′, AA, AA′, BB, BC, and BC′. In embodiments of the subject composition, the Kras G12D inhibitor has the formula selected from CF′ to CJ′ and any embodiments thereof, as disclosed herein, and the inhibitor of one or more signaling molecules selected from Table 1 is an inhibitor of SHP2 selected from

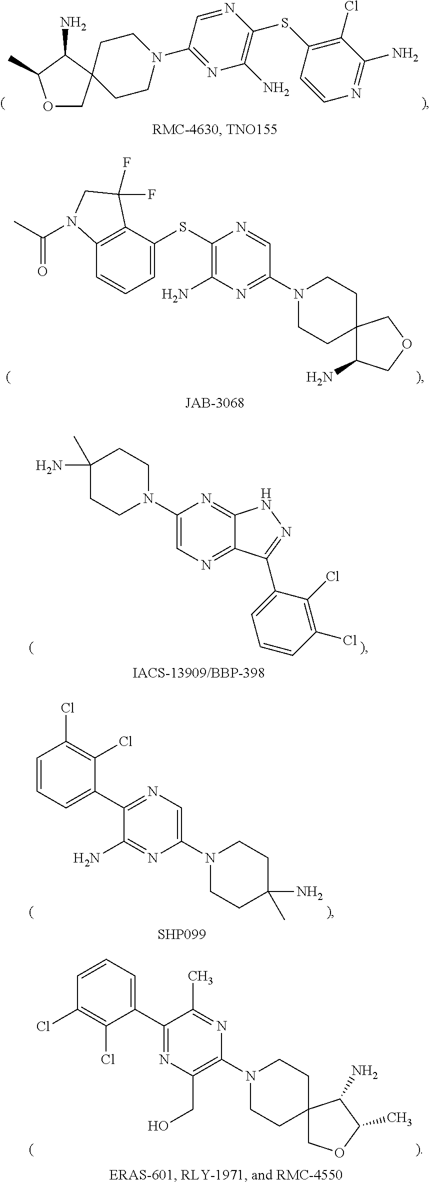 [SMILES: C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1 ; Cc1nc(N2CCC3(CC2)CO[C@@H](C)[C@H]3N)c(CO)nc1-c1cccc(Cl)c1Cl]

In embodiments of the subject composition, the Kras G12D inhibitor has the formula selected from CF′ to CJ′ and any embodiments thereof, and the inhibitor of one or more signaling molecules selected from Table 1 is an inhibitor of SOS selected from

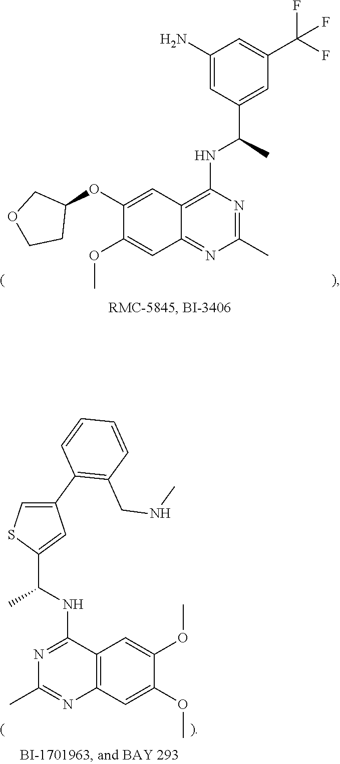

In embodiments of the subject composition, the Kras G12D inhibitor has the formula selected from CF′ to CJ′ and any embodiments thereof, as disclosed herein, and the inhibitor of one or more signaling molecules selected from Table 1 is an inhibitor of EGFR selected from afatinib, erlotinib, gefitinib, lapatinib, cetuximab panitumumab, osimertinib, olnutinib, and EGF-816. In embodiments of the subject composition, the Kras G12D inhibitor has the formula selected from CF′ to CJ′ and any embodiments thereof, as disclosed herein, and the inhibitor of one or more signaling molecules selected from Table 1 is an inhibitor of MEK selected from trametinib, cobimetinib, binimetinib, selumetinib, refametinib, and AZD6244. In embodiments of the subject composition, the Kras G12D inhibitor has the formula selected from CF′ to CJ′ and any embodiments thereof, as disclosed herein, and the inhibitor of one or more signaling molecules selected from Table 1 is an inhibitor of ERK selected from ulixertinib, MK-8353, LTT462, AZD0364, SCH772984, BIX02189, LY3214996, and ravoxertinib. In embodiments of the subject composition, the Kras G12D inhibitor has the formula selected from CF′ to CJ′ and any embodiments thereof, as disclosed herein, and the inhibitor of one or more signaling molecules selected from Table 1 is an inhibitor of CDK4/6 selected from palbociclib, ribociclib, and abemaciclib. In embodiments of the subject composition, the Kras G12D inhibitor has the formula selected from CF′ to CJ′ and any embodiments thereof, as disclosed herein, and the inhibitor of one or more signaling molecules selected from Table 1 is an inhibitor of BRAF selected from Sorafenib, Vemurafenib, Dabrafenib, Encorafenib, regorafenib, and GDC-879.

In embodiments of the subject composition, the Kras G12D inhibitor has a formula selected from formulae CF′ to CJ′, as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has a formula selected from formulae CF′ to CJ′, as disclosed herein, and the at least one inhibitor has a formula BB, BC, BC′, CF, CG, CH, CI, CJ, CK, and CK′. In embodiments of the subject composition, the Kras G12D inhibitor has a formula selected from formulae CF′ to CJ′, as disclosed herein, and the at least one inhibitor has a formula CL to CZ and DA to DZ. In embodiments of the subject composition, the combination comprises (a) a Kras G12D inhibitor having a formula selected from formulae CF′ to CJ′, as disclosed herein, and the inhibitor of EGFR is selected from afatinib, erlotinib, gefitinib, lapatinib, cetuximab panitumumab, osimertinib, olnutinib, and EGF-816. In embodiments of the subject composition, the Kras G12D inhibitor has a formula selected from formulae CF′ to CJ′, as disclosed herein, and the inhibitor of CDK4/6 is selected from palbociclib, ribociclib, and abemaciclib. In embodiments of the subject composition, the Kras G12D inhibitor has the formula (CF′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CF′), as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CF′), as disclosed herein, and the at least one inhibitor has a formula selected from formulae CL to CZ and DA to DZ. In embodiments of the subject composition, the Kras G12D inhibitor has the formula (CF′), as disclosed herein, and the inhibitor of EGFR is selected from afatinib, erlotinib, gefitinib, lapatinib, cetuximab panitumumab, osimertinib, olnutinib, and EGF-816. In embodiments of the subject composition, the Kras G12D inhibitor has the formula (CF′), as disclosed herein, and the inhibitor of CDK4/6 is selected from palbociclib, ribociclib, and abemaciclib. In embodiments of the subject composition, the Kras G12D inhibitor has the formula (CF′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CG′) or formula (CH′), as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CG′) or formula (CH′), as disclosed herein, and the at least one inhibitor has a formula selected from formulae CL to CZ and DA to DZ. In embodiments of the subject composition, the Kras G12D inhibitor has the formula (CG′) or formula (CH′), as disclosed herein, and the inhibitor of EGFR is selected from afatinib, erlotinib, gefitinib, lapatinib, cetuximab panitumumab, osimertinib, olnutinib, and EGF-816. In embodiments of the subject composition, the Kras G12D inhibitor has formula (CG′) or formula (CH′), as disclosed herein, and the inhibitor of CDK4/6 is selected from palbociclib, ribociclib, and abemaciclib. In embodiments of the subject composition, the Kras G12D inhibitor has the formula (CI′) as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CI′), as disclosed herein, and the at least one inhibitor has a formula selected from formulae CL to CZ and DA to DZ. In embodiments of the subject composition, the Kras G12D inhibitor has the formula (CI′), as disclosed herein, and the inhibitor of EGFR is selected from afatinib, erlotinib, gefitinib, lapatinib, cetuximab panitumumab, osimertinib, olmutinib, and EGF-816. In embodiments of the subject composition, the Kras G12D inhibitor has formula (CI′), as disclosed herein, and the inhibitor of CDK4/6 is selected from palbociclib, ribociclib, and abemaciclib. In embodiments of the subject composition, the Kras G12D inhibitor has the formula (CJ′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CJ′), as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CJ′), as disclosed herein, and the at least one inhibitor has a formula selected from formulae CL to CZ and DA to DZ. In embodiments of the subject composition, the Kras G12D inhibitor has the formula (CJ′), as disclosed herein, and the inhibitor of EGFR is selected from afatinib, erlotinib, gefitinib, lapatinib, cetuximab panitumumab, osimertinib, olnutinib, and EGF-816. In embodiments of the subject composition, the Kras G12D inhibitor has the formula (CJ′), as disclosed herein, and the inhibitor of CDK4/6 is selected from palbociclib, ribociclib, and abemaciclib. In embodiments of the subject composition, the Kras G12D inhibitor has the formula (CJ′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CH′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CH′), as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CH′), as disclosed herein, and the at least one inhibitor has a formula selected from formulae CL to CZ and DA to DZ. In embodiments of the subject composition, the Kras G12D inhibitor has the formula (CH′), as disclosed herein, and the inhibitor of EGFR is selected from afatinib, erlotinib, gefitinib, lapatinib, cetuximab panitumumab, osimertinib, olnutinib, and EGF-816. In embodiments of the subject composition, the Kras G12D inhibitor has the formula (CH′), as disclosed herein, and the inhibitor of CDK4/6 is selected from palbociclib, ribociclib, and abemaciclib. In embodiments of the subject composition, the Kras G12D inhibitor has the formula (CH′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CG′), as disclosed herein, and the at least one inhibitor has a formula selected from formulae CF to CZ, CK′, DA to DZ, A to F, N to Z, Z′, AA, AA′, BB, BC, and BC′. In embodiments of the subject composition, the Kras G12D inhibitor has the formula (CG′), as disclosed herein, and the at least one inhibitor has a formula selected from formulae BB, BC, BC′, CF, CG, CH, CI, CJ, CK, and CK′. In embodiments of the subject composition, the Kras G12D inhibitor has the formula (CG′), as disclosed herein, and the at least one inhibitor has a formula selected from formulae CL to CZ and DA to DZ. In embodiments of the subject composition, the Kras G12D inhibitor has the formula (CG′), as disclosed herein, and the inhibitor of EGFR is selected from afatinib, erlotinib, gefitinib, lapatinib, cetuximab panitumumab, osimertinib, olnutinib, and EGF-816. In embodiments of the subject composition, the Kras G12D inhibitor has the formula (CG′), as disclosed herein, and the inhibitor of CDK4/6 is selected from palbociclib, ribociclib, and abemaciclib. In embodiments of the subject composition, the Kras G12D inhibitor has the formula (CG′), as disclosed herein, and the at least one inhibitor has a formula selected from formulae CF to CZ, CK′, DA to DZ, A to F, N to Z, Z′, AA, AA′, BB, BC, and BC′.

In one aspect, the disclosure provides a method of inhibiting cell proliferation signaling in a cell, comprising: downregulating, in the cell, expression or activity of: (a) Kras G12D and (b) one or more signaling molecules selected from Table 1 and (c) Kras G12C.

In another aspect, the disclosure provides a modified cell characterized by exhibiting downregulated expression or activity of (a) Kras G12D and (b) one or more signaling molecules selected from Table 1 and (c) Kras G12C in accordance with the method described herein.

In yet another aspect, the disclosure provides a modified cell in which expression or activity of: (a) Kras G12D and (b) one or more signaling molecules selected from Table 1 and (c) Kras G12C is downregulated by an inhibitor against (a) and an inhibitor against (b) and an inhibitor against (c).

In some embodiments, the expression and/or activity of (i) Ras protein(s) (e.g., Kras G12D and/or Kras G12C) and that of (ii) one or more signaling molecules selected from Table 1 in a cell can be downregulated by at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control cell. The expression and/or activity of (i) the Ras protein(s) (e.g., Kras G12D and/or Kras G12C) and that of (ii) the one or more signaling molecules selected from Table 1 in the cell can be downregulated by at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less as compared to the control cell. The downregulation of expression and/or activity of (i) the Ras protein(s) (e.g., Kras G12D and/or Kras G12C) and that of (ii) the one or more signaling molecules selected from Table 1 in the cell can be maintained (or prolonged) for at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more longer as compared to a control cell. The downregulation of expression and/or activity of (i) the Ras protein(s) (e.g., Kras G12D and/or Kras G12C) and that of (ii) the one or more signaling molecules selected from Table 1 in the cell can be maintained for at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less longer as compared to the control cell.

In some embodiments, downregulating expression and/or activity of (a) Kras G12D and/or Kras G12C and that of (b) one or more signaling molecules selected from Table 1 reduces Ras signaling output in a cell. In some embodiments, the reduction in Ras signaling output is evidenced by one or more members of the following: (i) an increase in steady state level of GDP-bound Ras protein; (ii) a reduction of phosphorylated AKTs473, (iii) a reduction of phosphorylated ERKT202/y204, (iv) a reduction of phosphorylated S6S235/236, and (v) reduction (e.g., inhibition) of cell growth of the cell (e.g., a Ras-driven tumor cell, such as that derived from a tumor cell line). In some cases, the reduction in Ras signaling output can be evidenced by two or more members of (i)-(v). In some cases, the reduction in Ras signaling output can be evidenced by three or more members of (i)-(v). In some cases, the reduction in Ras signaling output can be evidenced by four or more members of (i)-(v). In some cases, the reduction in Ras signaling output can be evidenced by all of (i)-(v). GDP-bound Ras protein(s) (e.g., a GDP-bound Kras G12D and/or Kras G12C) may exhibit a lower degree of signaling activity (e.g., cell proliferation signaling activity) as compared to GTP-bound Ras protein(s) (e.g., a GTP-bound Kras G12D and/or Kras G12C).

In some embodiments, downregulating expression or activity of (a) Kras G12D and that of (b) one or more signaling molecules selected from Table 1 and (c) Kras G12C synergistically yields a greater degree of inhibition of cell proliferation in the cell as compared to (1) an individual degree of inhibition of cell proliferation via downregulating expression or activity of one of (a) and (b) and (c) (e.g., (a) alone or (b) alone or (c) alone) and/or (2) a sum of the individual degrees of inhibition of cell proliferation thereof.

In some examples, a degree of inhibition of cell proliferation in the cell by downregulating expression or activity of (a) and (b) and (c) is at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more greater than (1) an individual degree of inhibition of cell proliferation in a control cell by downregulating expression or activity of one of (a) and (b) and (c), alone and/or (2) a sum of the individual degrees of inhibition of cell proliferation thereof. In some examples, a degree of inhibition of cell proliferation in the cell by downregulating expression or activity of (a) and (b) and (c) is at least about at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less greater than (1) an individual degree of inhibition of cell proliferation in a control cell by downregulating expression or activity of one of (a) and (b) and (c), alone and/or (2) a sum of the individual degrees of inhibition of cell proliferation thereof. The inhibition of cell proliferation in the cell by downregulating expression or activity of (a) and (b) and (c) can be maintained (or prolonged) for at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more longer as compared to (1) individual inhibition of cell proliferation in a control cell by downregulating expression or activity of one of (a) and (b) and (c), alone and/or (2) a sum of the individual degrees of inhibition of cell proliferation thereof. The inhibition of cell proliferation in the cell by downregulating expression or activity of (a) and (b) and (c) can be maintained (or prolonged) for at most about 5000-fold, 4000-fold, 3000-fold, 2000-fold, 1000-fold, 900-fold, 800-fold, 700-fold, 600-fold, 500-fold, 400-fold, 300-fold, 200-fold, 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, 1-fold, 0.9-fold, 0.8-fold, 0.7-fold, 0.6-fold, 0.5-fold, 0.4-fold, 0.3-fold, 0.2-fold, 0.1-fold, or less longer as compared to (1) individual inhibition of cell proliferation in a control cell by downregulating expression or activity of one of (a) and (b) and (c), alone and/or (2) a sum of the individual degrees of inhibition of cell proliferation thereof.

In some cases, a control cell can be a cell that is not subjected to any treatment to downregulate expression and/or activity of (a) Kras G12D and that of (b) one or more signaling molecules selected from Table 1 and (c) Kras G12C. In some cases, a control cell can be a cell that is subjected to only one of: (a) an inhibitor against Kras G12D and (b) at least one additional inhibitor against one or more signaling molecules selected from Table 1 and (c) an inhibitor against Kras G12C. In some embodiments, expression or activity of a signal molecule (e.g., (a) Kras G12D and (b) one or more signaling molecules selected from Table 1 and (c) Kras G12C) is ascertained by a method selected from the group consisting of nucleic acid sequencing, in situ hybridization, immunohistochemistry (IHC), polymerase chain reaction (PCR), quantitative PCR (qPCR), quantitative real-time PCR (qRT-PCR), comparative genomic hybridization, microarray-based comparative genomic hybridization, and ligase chain reaction (LCR). In some embodiments, expression or activity of a signal molecule (e.g., (a) Kras G12D and (b) one or more signaling molecules selected from Table 1 and (c) Kras G12C) in a cell is permanently downregulated. In some embodiments, expression or activity of the signal molecule is transiently downregulated as compared to a control cell.

In some examples, the modified cell may have been treated with any of the inhibitors disclosed herein, e.g., an inhibitor against (a) a Ras protein (e.g., a mutant Ras, such as Kras G12D) and/or an inhibitor against (b) one or more signaling molecules selected from Table 1 and/or another inhibitor against (c) a different Ras protein from (a) (e.g., a mutant Ras, such as Kras G12C). In some embodiments, the modified cell exhibits reduced Ras signaling output in in the modified cell, as provided herein. In some examples, the modified cell has been treated with any of the pharmacologically active substances selected from Table 2. In some embodiments, the modified cell comprises any of the inhibitors disclosed herein, e.g., an inhibitor against (a) a Ras protein (e.g., a mutant Ras, such as Kras G12D) and/or an inhibitor against (b) one or more signaling molecules selected from Table 1 and/or an inhibitor (c) against a different Ras protein from (a) (e.g., a mutant Ras, such as Kras G12C).

Where desired, a subject (e.g., a human subject) can be screened for the presence of a Kras mutation, such as Kras G12C mutation. The subject can also be screened for the retention of expression and/or activity of a mutated Kras protein (e.g., Kras G12C) in one or more types of subject's cells (e.g., cancer cells).

### Compositions

In some embodiments, the composition comprises one or more inhibitors capable of downregulating expression and/or activity of (a) a Ras protein (e.g., a mutated Ras protein, such as Kras G12D) and that of (b) one or more signaling molecules selected from Table 1 and that of (c) against a different Ras protein from (a) (e.g., a mutant Ras, such as Kras G12C), in accordance with the method of any one of the preceding claims. In some embodiments, the present disclosure provides a composition comprising: an inhibitor against (a) a Ras protein (e.g., a mutated Ras protein, such as Kras G12D); and/or an inhibitor against (b) one or more signaling molecules selected from Table 1 and/or an inhibitor against (c) a different Ras protein from (a) (e.g., a mutant Ras, such as Kras G12C).

In embodiments, the inhibitor of Kras G12C is adagrasib

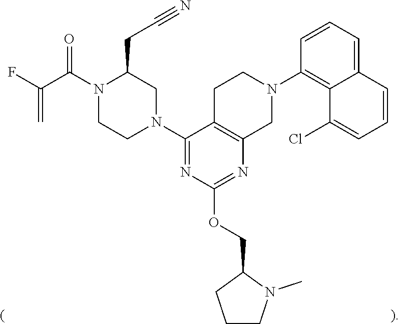 [SMILES: C=C(F)C(=O)N1CCN(c2nc(OC[C@@H]3CCCN3C)nc3c2CCN(c2cccc4cccc(Cl)c24)C3)C[C@@H]1CC#N]

In embodiments, the inhibitor of Kras G12C is MRTX849. In embodiments, the inhibitor of Kras G12C is sotorasib

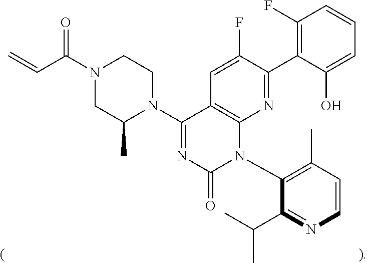

In embodiments, the inhibitor of Kras G12C is AMG 510. In embodiments, the inhibitor of Kras G12C is RMC-6291, RMC-6236, or JNJ-74699157 (ARS-3248).

Another aspect of the present disclosure provides one or more inhibitor compounds against one or more targets selected from the group comprising: (a) a Ras protein (e.g., a mutated Ras protein, such as Kras G12D) and (b) one or more signaling molecules selected from Table 1. A different aspect of the present disclosure provides a single inhibitor compound comprising a plurality (e.g., at least 2, 3, 4, 5, or more) of inhibitors against the same target selected from the group comprising: (a) a Ras protein (e.g., a mutated Ras protein, such as Kras G12D) and (b) one or more signaling molecules selected from Table 1. Yet a different aspect of the present disclosure provides a single inhibitor compound against a plurality (e.g., 2, 3, 4, 5, or more) of targets selected from the group comprising: (a) a Ras protein (e.g., a mutated Ras protein, such as Kras G12D) and (b) one or more signaling molecules selected from Table 1. In some embodiments, a single inhibitor compound can inhibit expression and/or activity of two or more targets selected from: (a) a Ras protein (e.g., a mutated Ras protein, such as Kras G12D) and an inhibitor against (b) one or more signaling molecules selected from Table 1. In some embodiments, a single inhibitor compound comprises two or more different inhibitors disclosed herein. In some examples, a first inhibitor against (a) a Ras protein (e.g., a mutated Ras protein, such as Kras G12D) and a second inhibitor against (b) one or more signaling molecules selected from Table 1 are coupled to each other (e.g., directly or indirectly) to form a single compound. In some examples, a first inhibitor against (a) a signaling molecule selected from Table 1 and a second inhibitor against (b) a different signaling molecule selected from Table 1 are coupled to each other (e.g., directly or indirectly) to form a single compound. A single compound disclosed herein can be a small molecule, a nucleic acid agent, or a polypeptide, as provided herein.

In some embodiments, a single compound has the structure A-L-A, wherein each A is an inhibitor against the same target (e.g., Kras G12D), and L is a bond or any linker described herein. In some embodiments, a single compound has the structure A-L-B, wherein A and B are distinct and different inhibitors against different targets (e.g., different targets selected from: (a) a Ras protein (e.g., a mutated Ras protein, such as Kras G12D) and (b) one or more signaling molecules selected from Table 1), and L is a bond or any linker described herein. In an example, A or B is capable of inhibiting activity of a Ras protein (e.g., Kras G12D) by binding to the Ras protein. In another example, A or B is capable of inhibiting activity of one or more signaling molecules (e.g., selected from Table 1 by binding to the one or more signaling molecules).

L groups

In some embodiments of the inhibitor disclosed herein (e.g., having a A-L-A or A-L-B structure), L is a bond.

In some embodiments of the inhibitor disclosed herein (e.g., having a A-L-A or A-L-B structure), L is a linker. In some embodiments of the inhibitor disclosed herein (e.g., having a A-L-A or A-L-B structure), L is a linker comprising 1 to 50 non-hydrogen atoms. In some embodiments of the inhibitor disclosed herein, L is a linker comprising 1 to 40 non-hydrogen atoms. In some embodiments of the inhibitor disclosed herein, L is a linker comprising 1 to 30 non-hydrogen atoms. In some embodiments of the inhibitor disclosed herein, L is a linker comprising 1 to 20 non-hydrogen atoms. In some embodiments of the inhibitor disclosed herein, L is a linker comprising 1 to 10 non-hydrogen atoms.

In some embodiments of the inhibitor disclosed herein, L is a linker moiety comprising 1 or more groups, in a branched or linear configuration, independently selected from alkyl, alkenyl, alkynyl, alkoxy, aryl, heteroaryl, heterocyclylalkyl, cycloalkyl, 0, S, N, halo, hydroxyl, amino, cyano, and oxo. In some embodiments of the inhibitor disclosed herein, L is a linker moiety comprising 1 or more groups, in a branched or linear configuration, independently selected from alkyl, alkenyl, alkynyl, alkoxy, O, S, N, halo, hydroxyl, amino, cyano, and oxo.

In some embodiments, the linker is as described in WO2019195609 and WO2020018788, or related patents and applications, each of which is incorporated by reference in its entirety.

### Kits

Another aspect of the present disclosure provides a kit comprising: the composition of any one of the preceding claims; and instructions directing (i) contacting a cell with any composition disclosed herein and/or (ii) administration of any composition disclosed herein to a subject in need thereof. The composition of the kit can comprise at least about 1, 2, 3, 4, 5, 6, 7, 8, 9, 10, 11, 12, 13, 14, 15, 16, 17, 18, 19, 20, 25, 30, 35, 40, 45, 50, or more unit dosages of any inhibitor disclosed herein. The composition of the kit can comprise at most about 50, 45, 40, 35, 30, 25, 20, 19, 18, 17, 16, 15, 14, 13, 12, 11, 10, 9, 8, 7, 6, 5, 4, 3, 2, or 1 unit dosage of any inhibitor disclosed herein. In some embodiments, two or more inhibitors as disclosed herein (e.g., a Kras G12D inhibitor and at least one inhibitor of a signaling molecule selected from Table 1) can be in a same unit dosage (e.g., same liquid formulation, same tablet, etc.). In some embodiments, the two or more inhibitors as disclosed herein can be in different unit dosages (e.g., in different pharmaceutical composition forms, in different liquid formulations, in different tablets, etc.).

In some embodiments, the kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with other dietary supplements, vitamins, and meal replacement bars, mixes, and beverages.

### EXAMPLES

### Example 1: Lenti-Viral Preparation and Infection to Target Cells

Lentiviral Preparation:

Nucleic acid agents, e.g., shRNA, can be used as an inhibitor to downregulate expression of a signal molecule (e.g., a polypeptide) in a cell. In an example, a signal molecule can comprise (a) Kras G12D and (b) SOS, SHIP2, MEK, ERK, or EGFR The cell can be contacted by such shRNA, e.g., by transfecting the cell with a lenti-virus comprising a gene encoding the shRNA. Nucleic acid agents, e.g., shRNA, can be used as an inhibitor to downregulate expression of a signal molecule (e.g., a polypeptide) in a cell. In an example, a signal molecule can comprise (a) Kras G12D and (b) SOS, SHP2, MEK, ERK, or EGFR The cell can be contacted by such shRNA, e.g., by transfecting the cell with a lenti-virus comprising a gene encoding the shRNA.

Lenti-viruses are prepared from 293T cells. Briefly, approximately 10 million 293T cells are seeded onto collagen coated 15 cm dishes at day −1. At day 0, approximately 10-20 ug shRNA vector (e.g., (i) a vector comprising a sequence encoding shRNA targeting Kras G12D, (ii) a vector comprising a sequence encoding shRNA against SOS, SHIP2, MEK, ERK, or EGFR, and/or (iii) a vector encoding a control shRNA), 15 ug Gag/pol vector, and 5 ug VSV-G vector are transfected using Lipofectamin 2000 (Invitrogen). 24 hours later (day 1), media is changed. After changing media, viral supernants are harvested at day 2 and day 3. Viruses are concentrated with Lenti-X concentrator.

Lenti-viruses are prepared from 293T cells. Briefly, approximately 10 million 293T cells are seeded onto collagen coated 15 cm dishes at day −1. At day 0, approximately 10-20 ug shRNA vector (e.g., (i) a vector comprising a sequence encoding shRNA targeting Kras G12D, (ii) a vector comprising a sequence encoding shRNA against SOS, SHP2, MEK, ERK, or EGFR, and/or (iii) a vector encoding a control shRNA), 15 ug Gag/pol vector, and 5 ug VSV-G vector are transfected using Lipofectamin 2000 (Invitrogen). 24 hours later (day 1), media is changed. After changing media, viral supernants are harvested at day 2 and day 3. Viruses are concentrated with Lenti-X concentrator.

Infection to Cancer Cells:

ASPC-1 (ATCC CRL-1682) and Panc-10.05 (ATCC CRL-2547) cancer cell lines comprise a G12D mutation and can be used to assess downregulation of expression or activity of the signal molecule, e.g., in response to the shRNA disclosed herein.

ASPC-1 culture medium is prepared with RPMI-1640 and 10% heat-inactivated FBS. Panc-10.05 culture medium is prepared with RPMI-1640, 10 Units/m1 human recombinant insulin, and 15% FBS.

Approximately 100 ul of viruses are added into approx. 0.5 million cancer cells in the presence of 6 ug/ml of polybrene. The cells are spin-infected at 2000 rpm, 90 min at 32° C. After 1 hour incubation at 37° C. incubator, fresh RPMI 1640 media is added and transferred into 24-well plate. At day 6, cells is transferred into 6-well plate in the presence of final concentration about 2 ug/ml of puromycin for about 5 days

### Example 2: Inhibition by shRNA Inhibitor

Western Blotting: Demonstrating Inhibition of Signal Molecules

Inhibition of signal molecule expression is carried out with the use of each shRNA against a gene encoding the signal molecule, which is transfected into cancer cells. For instance, shRNA against a gene encoding Kras G12D and shRNA against a gene encoding SOS, SHIP2, MEK, ERK, or EGFR are used in combination. For instance, shRNA against a gene encoding Kras G12D and shRNA against a gene encoding SOS, SHP2, MEK, ERK, or EGFR are used in combination. Total cell lysates are prepared in protease inhibitor cocktails (Sigma) containing RIPA buffer. Protein concentration is measured by e.g., BCA protein assay kits (Pierce, Item #: 3603904). Total cell lysate protein is subjected to SDS-PAGE followed by transferring protein onto nitrocellulose membrane using iBot transfer system (Invitrogen, 20V, 11 min 30 sec). The membrane is blocked in standard 5% BSA containing TBST for approximately 30 min at room temperature. Antibodies against the signal molecule (e.g., Cell Signaling Technology Cat #14429S, Cat #5890S, Cat #2839S, Cat #9127S, Cat #9101S, or Cat #3197S) is incubated with membranes. Signal molecule level is detected according to the manufacturer's instruction.

qRT-PCR: Demonstrating Inhibition of Signal Molecule Expression

qRT-PCR is utilized to assess inhibition of signal molecule expression shRNA against a gene encoding the signal molecule, which is transfected into cancer cells. For instance, shRNA against a gene encoding Kras G12D and shRNA against a gene encoding SOS, SHIP2, MEK, ERK, or EGFR are used in combination. For instance, shRNA against a gene encoding Kras G12D and shRNA against a gene encoding SOS, SHP2, MEK, ERK, or EGFR are used in combination. An RNeasy Micro Kit (Qiagen) is used to extract RNA. mRNA is reverse transcribed to single-strand complementary DNA (cDNA) with SuperScript III First-Strand Synthesis System for RT-PCR (Invitrogen). Real-time PCR is performed with C1000 Touch Thermal Cycler (Biorad). A SYBR-based protocol is used to detect gene expression (SsoAdvanced Universal SYBR Green Supermix, Biorad). The PCR reactions are done in 96-well plates and run using the manufacture's recommended cycling parameters using primers hybridizing to the coding region of signal molecule polypeptide.

Scrambled control shRNA is utilized as a negative control in parallel to each shRNA. The shRNA constructs expressing Red Fluorescence Protein (RPF) and puromycin resistant gene are introduced into the cancer cells to demonstrate knockdown efficiency of shRNAs against signal molecule genes by qRT-PCR or Western Blotting, shRNA infected cancer cells expressing RFP can also be detected and quantitated by FACS on FACS Fortessa (BD), with the aid of FlowJo (Treestar Inc.) program.

### Example 3: CRISPR-Based Signal Molecule Inhibitor

Guide RNA (gRNA) molecules comprising the targeting sequences exhibiting homology to (a) a polynucleotide encoding Kras G12D and (b) a polynucleotide encoding SOS, SHIP2, MEK, ERK, or EGFR are designed according to methods known in the art. gRNA can be directed to the coding or regulatory sequences of such signal molecules to effect specific targeting. Guide RNA (gRNA) molecules comprising the targeting sequences exhibiting homology to (a) a polynucleotide encoding Kras G12D and (b) a polynucleotide encoding SOS, SHP2, MEK, ERK, or EGFR are designed according to methods known in the art. gRNA can be directed to the coding or regulatory sequences of such signal molecules to effect specific targeting.

Introduction of CRISPR-Based Signal Molecule Inhibitor into Cancer Cells

Isolated and frozen cancer cells (e.g., ASPC-1 and Panc-10.05 cell lines, as disclosed herein) are thawed on day 0. Between days 1 through 3, cancer cells are electroporated to introduce CRISPR/Cas systems in the form of pre-complexed gRNA/Cas9 ribonuclear protein (“RNP”). The cells are allowed to grow in culture for approximately one more week. Cells are then divided, some being used for flow cytometry to stain a presence or change in expression of the signal molecules by using antibodies (e.g., Cell Signaling Technology Cat #14429S, Cat #5890S, Cat #2839S, Cat #9127S, Cat #9101S, or Cat #3197S). A portion of the remaining cells is used for next generation sequencing (NGS) on Illumina platform to confirm cleavage of endogenous sequences encoding the signal molecules in the cells. The rest of the cells can be kept for other functional assays.

### Example 4: ERK Phosphorylation Assay

The reduction in Ras signaling output by the inhibitor of the present disclosure can be determined by measuring the amount of a downstream marker of Ras activity, such as the phosphorylated ERK (Phospho-ERK). For example, the inhibitor can comprise a small molecule inhibitor against (a) Kras G12D and a small molecule inhibitor against (b) SOS, SHIP2, MEK, ERK, or EGFR For example, the inhibitor can comprise a small molecule inhibitor against (a) Kras G12D and a small molecule inhibitor against (b) SOS, SHP2, MEK, ERK, or EGFR

ASPC-1 cell line (ATCC CRL-1682) or Panc-10.05 cell line (ATCC CRL-2547) expressing K-Ras G12D is used. Cells are plated in poly-D-Lysine coated 96-well plates at a concentration of 50,000 cells/well and allowed to attach for 8-12 hours. Following, diluted solutions of each inhibitor are added to the cell culture at a final concentration of 0.5% DMSO. After 3 hours, the medium is removed, a 150 μL of 4% formaldehyde is added, and the plates is incubated for 20 minutes. The plates can be washed with PBS, and permeabilized using 150 μL of ice cold 100% methanol for 10 minutes. Non-specific antibody binding to the plates is blocked using 100 μL Licor Blocking Buffer (Li-Cor Biotechnology, Lincoln Nebr.) for 1 hour at room temperature. Positive control samples and samples lacking cells can be processed in parallel with test samples as standards.

The amount Phospho-ERK can be determined using an antibody specific for the phosphorylated form of ERK and compared to the amount of a housekeeping protein, such as GAPDH. Examples of the primary antibodies used for detection are as follows: Phospho-ERK (Cell Signaling cs9101) diluted 1:500 and GAPDH (Millipore MAB374) diluted 1:5000 in Licor block+0.05% Tween 20. The plates are incubated for 2 hours at room temperature. The plates are washed with PBS+0.05% Tween 20.

Examples of the secondary antibodies used to visualize the primary antibodies are as follows: Anti-rabbit-680 diluted 1:1000 and Anti-mouse-800 diluted 1:1000 in Licor Block+0.05% Tween 20 and incubated for 1 hour at room temperature. The plates are washed with PBS+0.05% Tween 20. A 100 μL aliquot of PBS are added to each well and the plates can be read on a plate reader, such as a LICOR AERIUS plate reader.

The pERK (Thr202/Tyr204) signal is normalized with the GAPDH signal, and percent of DMSO control values can be calculated. IC50 values are generated using a 4 parameter fit of the dose response curve. The resulting IC50 value can be a measurement of the ability of the inhibitor to reduce Ras signaling output in a cell.

### Example 5: Cell Proliferation Assay

ASPC-1 (ATCC CRL-1682) and Panc-10.05 (ATCC CRL-2547) cell lines comprise a G12D mutation and can be used to assess Ras cellular signaling in vitro, e.g., in response to the inhibitor of the present disclosure. For example, the inhibitor can comprise a small molecule inhibitor against (a) Kras G12D and a small molecule inhibitor against (b) SOS, SHIP2, MEK, ERK, or EGFR. For example, the inhibitor can comprise a small molecule inhibitor against (a) Kras G12D and a small molecule inhibitor against (b) SOS, SHP2, MEK, ERK, or EGFR

A CellTiter-Glo (CTG) luminescent based assay (Promega) is used to assess growth of the cells, as a measurement of the ability of each inhibitor to inhibit Ras signaling in the cells. The cells (e.g., 800-1,200 per well) are seeded in their respective culture medium in standard tissue culture-treated 96-well format plates (Corning Costar #3903) or ultra-low attachment surface 96-well format plates (Corning Costar #3474). The day after plating, cells are treated with a dilution series (e.g., a 9 point 3-fold dilution series) of each inhibitor (˜100 μl final volume per well). Cell viability can be monitored (˜5 days later) according to the manufacturer's recommended instructions, where the CellTiter-Glo reagent is added (˜50 μl), vigorously mixed, covered, and placed on a plate shaker (˜20 min) to ensure sufficient cell lysis prior to assessment of luminescent signal.

### Example 6: Nucleic-Acid Based Kras G12D Inhibitor, with SOS Inhibitor

This study explores the inhibition of a variety types (e.g., Kras G12D driven tumor cells) of cancer cells using a combination of nucleic-acid based Kras12D inhibitor and SOS inhibitor. Cell lines utilized include A427, a non-small cell lung cancer (NSCLC), ASPC1, a pancreatic cancer, and Ls513, colorectal cancer cell line. All three cell lines contain a KRAS G12D mutation. The cancer cell lines were adherently cultured in vitro and transduced with lentiviral particles packaged with shRNAs targeting either KRAS or negative control gene Luciferase.

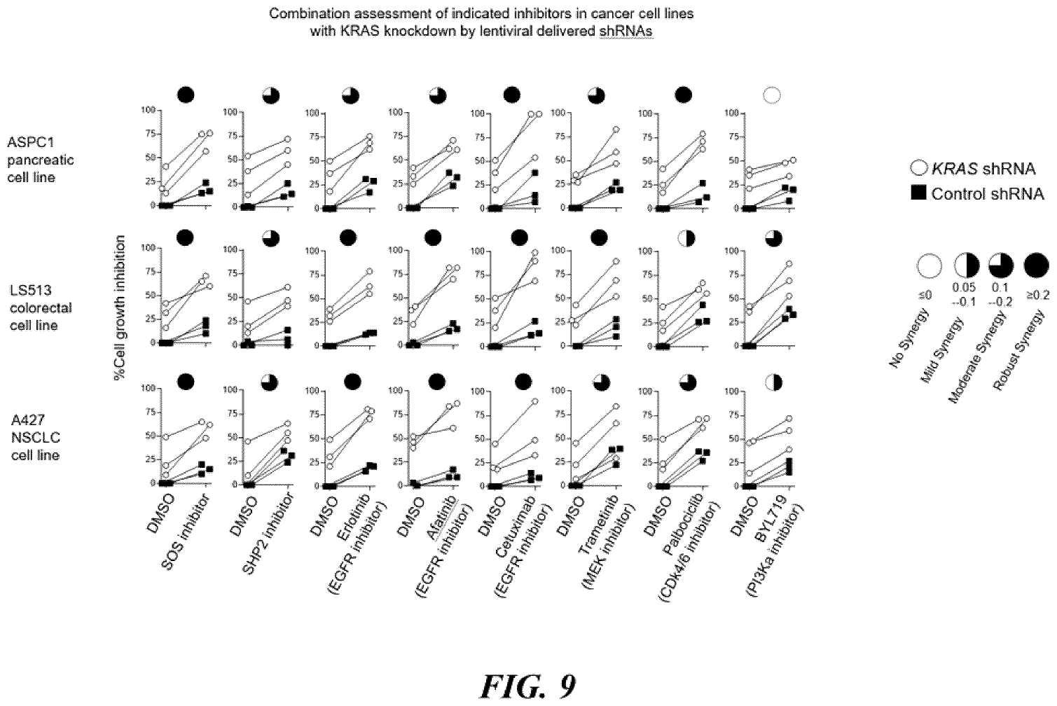

Amphotropic lentiviruses were produced by the co-transfection of 293FT cells and were packaged using the ViraPower lentiviral expression system (ThermoFisher Scientific). Lentiviral shRNA clones targeting luciferase (as sh-Ctrl) and KRAS were obtained from Sigma in the MISSION pLKO.1-puro vector with clone IDs as follows: shKRAS #1: TRCN0000033262, NM_033360.2-509s1c1, shKRAS #2: TRCN0000033260, NM_033360.2-407s1c1, and the Luciferase shRNA control plasmid DNA (Cat #SHC007, Sigma). Viral supernatants were filtered through a 0.45 pm filter and supplemented with 8 pg m1-1 of polybrene (Sigma) and added to 70% confluent cells pre-plated in six-well dishes. Plates were spinfected for 1 h under 600×g at 37° C. centrifugation. Cells were subsequently washed with PBS and allowed to rest 48 hours in fresh culture media at 37° C. Next, cells were reseeded as 3D suspensions (ultra-low adherent plates) into 384 well plates in RPMI medium containing 10% FBS. Approximately 24 hrs later, 10 uM SOS inhibitor as described herein or DMSO (as negative control) was added to the cell cultures for a period of about 5 days. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIG. 9, first column of graphs, the degree of enhanced cell growth inhibition is displayed for the nucleic-acid based Kras12D inhibitor alone, SOS inhibitor alone, or in combination with each other respectively. The degree of synergy for the combination across cell lines was calculated by using the Bliss independence model. The degree of excess over Bliss is the difference in cell growth inhibition between the experimentally observed and the predicted inhibition at various compound concentrations whereby values >0 are indicative of more than additive effects and are instead synergistic. The greater the departure from Bliss indicates more profound synergy: <0 is no synergism, 0.05-0.1 is mild synergy, 0.1-0.2 moderate synergy, and >0.2 is robust synergy. The assessment of the combinatorial effect using the Bliss model is displayed for each combination displayed as a pie chart. The nucleic-acid based Kras12D inhibitor in combination with a SOS inhibitor revealed robust synergistic cell growth inhibition across all cell lines tested as assessed in accordance to the Bliss independence model exemplified herein.

### Example 7: Nucleic-Acid Based Kras G12D Inhibitor, with SHP2 Inhibitor

This study explores the inhibition of a variety types (e.g., Kras G12D driven tumor cells) of cancer cells using a combination of nucleic-acid based Kras12D inhibitor and SHP2 inhibitor. Cell lines utilized include A427, a non-small cell lung cancer (NSCLC), ASPC1, a pancreatic cancer, and Ls513, colorectal cancer cell line. All three cell lines contain a KRAS G12D mutation. The cancer cell lines were adherently cultured in vitro and transduced with lentiviral particles packaged with shRNAs targeting either KRAS or negative control gene Luciferase.

Amphotropic lentiviruses were produced in the same manner as described in the preceding Example 6. 1 uM SHP2 inhibitor as described herein or DMSO (as negative control) was added to the cell cultures for a period of about 5 days. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIG. 9, second column of graphs, the degree of enhanced cell growth inhibition is displayed for the nucleic-acid based Kras12D inhibitor alone, SHP2 inhibitor alone, or in combination with each other respectively. The degree of synergy for the combination across cell lines was calculated by using the Bliss independence model. The degree of excess over Bliss is the difference in cell growth inhibition between the experimentally observed and the predicted inhibition at various compound concentrations whereby values >0 are indicative of more than additive effects and are instead synergistic. The greater the departure from Bliss indicates more profound synergy: <0 is no synergism, 0.05-0.1 is mild synergy, 0.1-0.2 moderate synergy, and >0.2 is robust synergy. The assessment of the combinatorial effect using the Bliss model is displayed for each combination displayed as a pie chart. The nucleic-acid based Kras12D inhibitor in combination with a SHP2 inhibitor revealed moderate synergistic cell growth inhibition across all cell lines tested as assessed in accordance to the Bliss independence model exemplified herein.

### Example 8: Nucleic-Acid Based Kras G12D Inhibitor, with EGFR Inhibitor

This study explores the inhibition of a variety types (e.g., Kras G12D driven tumor cells) of cancer cells using a combination of nucleic-acid based Kras12D inhibitor and EGFR inhibitor, either erlotinib, afatinib, or cetuximab. Cell lines utilized include A427, a non-small cell lung cancer (NSCLC), ASPC1, a pancreatic cancer, and Ls513, colorectal cancer cell line. All three cell lines contain a KRAS G12D mutation. The cancer cell lines were adherently cultured in vitro and transduced with lentiviral particles packaged with shRNAs targeting either KRAS or negative control gene Luciferase.

Amphotropic lentiviruses were produced in the same manner as described in the preceding Example 6. Cells were then incubated with either 3 uM of erlotinib, 0.1 uM of afatinib, or 0.1 uM of cetuximab, respectively for a period of about 5 days. The concentration of the respective EGFR inhibitors utilized in this cell assay was chosen to be within the range corresponding to achievable exposures and pharmacokinetics of a recommended and clinically relevant doses applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIG. 9, third to fifth columns of graphs, the degree of enhanced cell growth inhibition is displayed for the nucleic-acid based Kras12D inhibitor alone, EGFR inhibitor alone, or in combination with each other respectively. The degree of synergy for the combination across cell lines was calculated by using the Bliss independence model as exemplified in the preceding Example 6. The nucleic-acid based Kras12D inhibitor in combination with one of three distinct EGFR inhibitors revealed moderate to robust synergistic cell growth inhibition across all cell lines tested as assessed in accordance to the Bliss independence model exemplified herein. As single agents, the concentrations of single agent EGFR inhibitors (3 uM of erlotinib, 0.1 uM of afatinib, or 0.1 uM of cetuximab) elicited minimal growth inhibition (<30%). The nucleic-acid based Kras12D inhibitor in combination with erlotinib or afatinib boosted growth inhibition up to 75% in pancreatic, and colorectal and NSCLC cell lines. The nucleic-acid based Kras12D inhibitor in combination with cetuximab significantly boosted growth inhibition up to 100% in pancreatic, and colorectal and NSCLC cell lines.

### Example 9: Nucleic-Acid Based Kras G12D Inhibitor, with MEK Inhibitor

This study explores the inhibition of a variety types (e.g., Kras G12D driven tumor cells) of cancer cells using a combination of nucleic-acid based Kras12D inhibitor and a MEK inhibitor, Trametinib. Cell lines utilized include A427, a non-small cell lung cancer (NSCLC), ASPC1, a pancreatic cancer, and Ls513, colorectal cancer cell line. All three cell lines contain a KRAS G12D mutation. The cancer cell lines were adherently cultured in vitro and transduced with lentiviral particles packaged with shRNAs targeting either KRAS or negative control gene Luciferase.

Amphotropic lentiviruses were produced in the same manner as described in the preceding Example 6. 3 nM of Trametinib or DMSO (negative control) was added to the cell cultures for a period of about 5 days. The concentration of the MEK inhibitor utilized in this cell assay was chosen to be within the range corresponding to achievable exposures and pharmacokinetics of a recommended and clinical relevant dose applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIG. 9, sixth column of graphs, the degree of enhanced cell growth inhibition is displayed for the nucleic-acid based Kras12D inhibitor alone, MEK inhibitor alone, or in combination with each other respectively. The degree of synergy for the combination across cell lines was calculated by using the Bliss independence model as exemplified in the preceding Example 6. The nucleic-acid based Kras12D inhibitor in combination with MEK inhibitor revealed moderate to robust synergistic cell growth inhibition across all cell lines tested as assessed in accordance to the Bliss independence model exemplified herein. The combination elicited up to 89% cell growth inhibition, whereas single agent activity was only capable of achieving <50% cell growth inhibition.

### Example 10: Nucleic-Acid Based Kras G12D Inhibitor, with CDK4/6 Inhibitor

This study explores the inhibition of a variety types (e.g., Kras G12D driven tumor cells) of cancer cells using a combination of nucleic-acid based Kras12D inhibitor and a CDK4/6 inhibitor, namely Palbociclib. Cell lines utilized include A427, a non-small cell lung cancer (NSCLC), ASPC1, a pancreatic cancer, and Ls513, colorectal cancer cell line. All three cell lines contain a KRAS G12D mutation. The cancer cell lines were adherently cultured in vitro and transduced with lentiviral particles packaged with shRNAs targeting either KRAS or negative control gene Luciferase.

Amphotropic lentiviruses were produced in the same manner as described in the preceding Example 6. Cells were then incubated with 0.3 uM of CDK4/6 inhibitor or DMSO (negative control) for a period of 5 days. The concentration of the CDK4/6 inhibitor utilized in this cell assay was chosen to be within the range corresponding to achievable exposures and pharmacokinetics of a recommended and clinical relevant dose applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIG. 9, seventh column of graphs, the degree of enhanced cell growth inhibition is displayed for the nucleic-acid based Kras12D inhibitor alone, CDK4/6 inhibitor alone, or in combination with each other respectively. The degree of synergy for the combination across cell lines was calculated by using the Bliss independence model as exemplified in the preceding Example 6. The nucleic-acid based Kras12D inhibitor in combination with CDK4/6 inhibitor revealed mild to robust synergistic cell growth inhibition across all cell lines tested (see pie charts in seventh column of graphs in FIG. 9) as assessed in accordance to the Bliss independence model exemplified herein. The combination elicited up to about 79% cell growth inhibition, whereas single agent activity was only capable of achieving <50% cell growth inhibition.

### Example 11: Nucleic-Acid Based Kras G12D Inhibitor, with PI3K Inhibitor

This study explores the inhibition of a variety types (e.g., Kras G12D driven tumor cells) of cancer cells using a combination of nucleic-acid based Kras12D inhibitor and a PI3K-alpha inhibitor, namely BYL719. Cell lines utilized include A427, a non-small cell lung cancer (NSCLC), ASPC1, a pancreatic cancer, and Ls513, colorectal cancer cell line. All three cell lines contain a KRAS G12D mutation. The cancer cell lines were adherently cultured in vitro and transduced with lentiviral particles packaged with shRNAs targeting either KRAS or negative control gene Luciferase.

Amphotropic lentiviruses were produced in the same manner as described in the preceding Example 6. Cells were then incubated with 0.3 uM of PI3K-alpha inhibitor or DMSO (negative control) for a period of 5 days. The concentration of the PI3K-alpha inhibitor utilized in this cell assay was chosen to be within the range corresponding to achievable exposures and pharmacokinetics of a recommended and clinical relevant dose applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIG. 9, eighth column of graphs, the degree of enhanced cell growth inhibition is displayed for the nucleic-acid based Kras12D inhibitor alone, PI3K-alpha inhibitor alone, or in combination with each other respectively. The degree of synergy for the combination across cell lines was calculated by using the Bliss independence model as exemplified in the preceding Example 6. The nucleic-acid based Kras12D inhibitor in combination with PI3K-alpha inhibitor revealed little to moderate synergistic cell growth inhibition across all cell lines tested (see pie charts in eighth column of graphs in FIG. 9) as assessed in accordance to the Bliss independence model exemplified herein. The combination elicited up to 87% cell growth inhibition, whereas single agent activity was only capable of achieving <50% cell growth inhibition.

### Example 12: Small-Molecule Based Kras G12D Inhibitor, with SOS Inhibitor

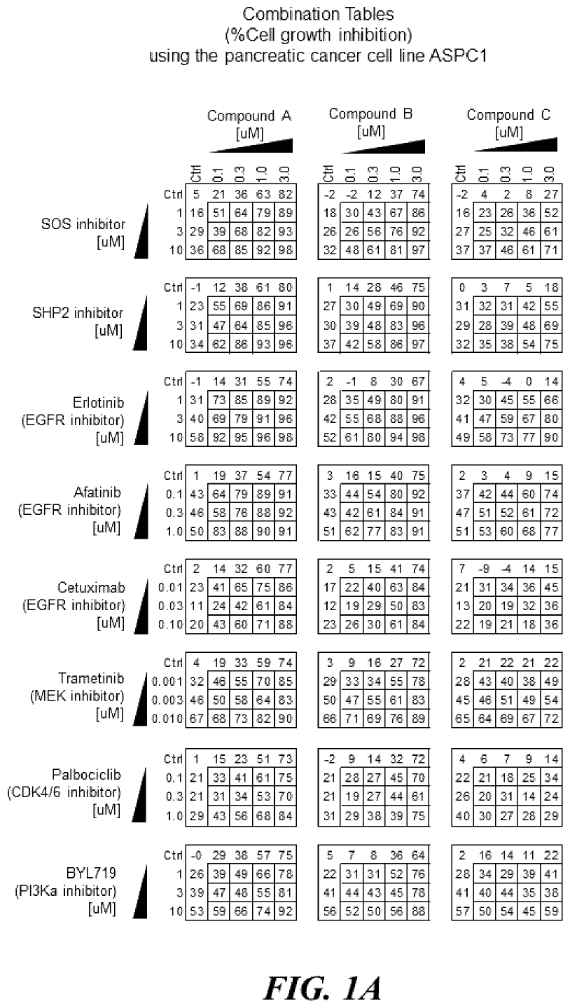

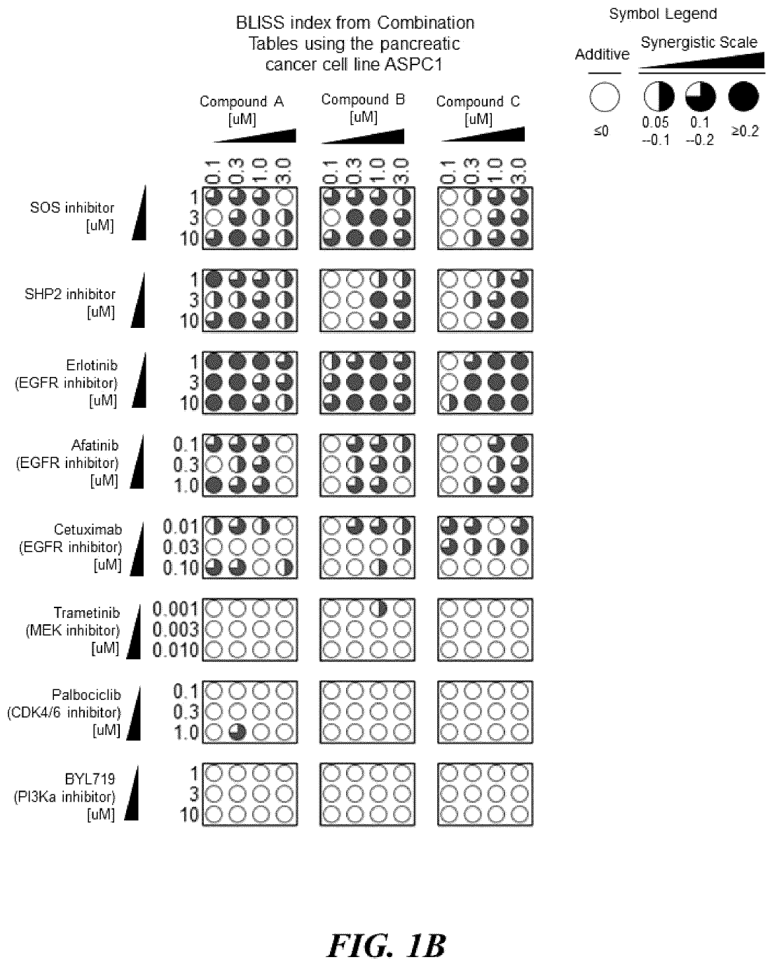

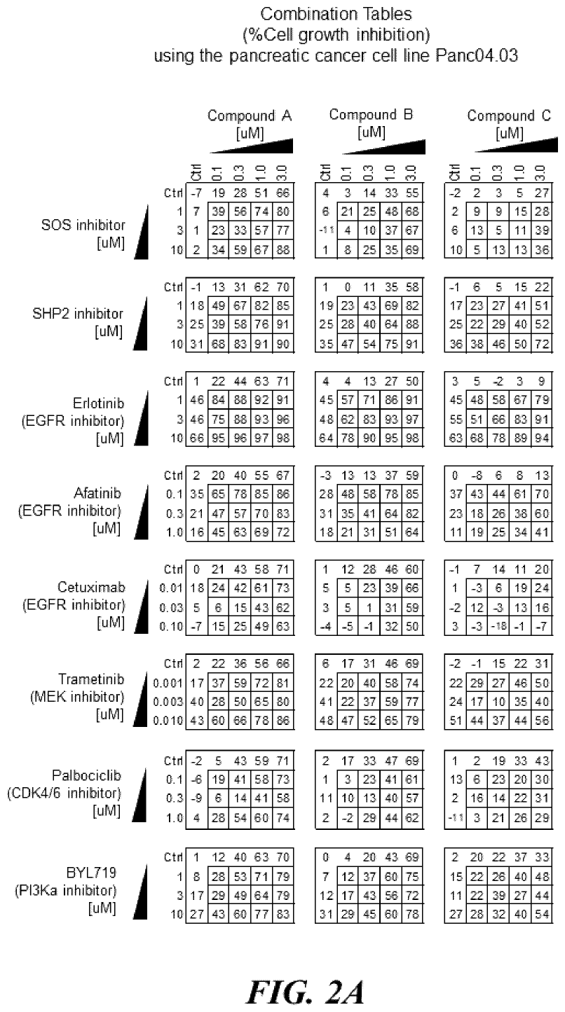

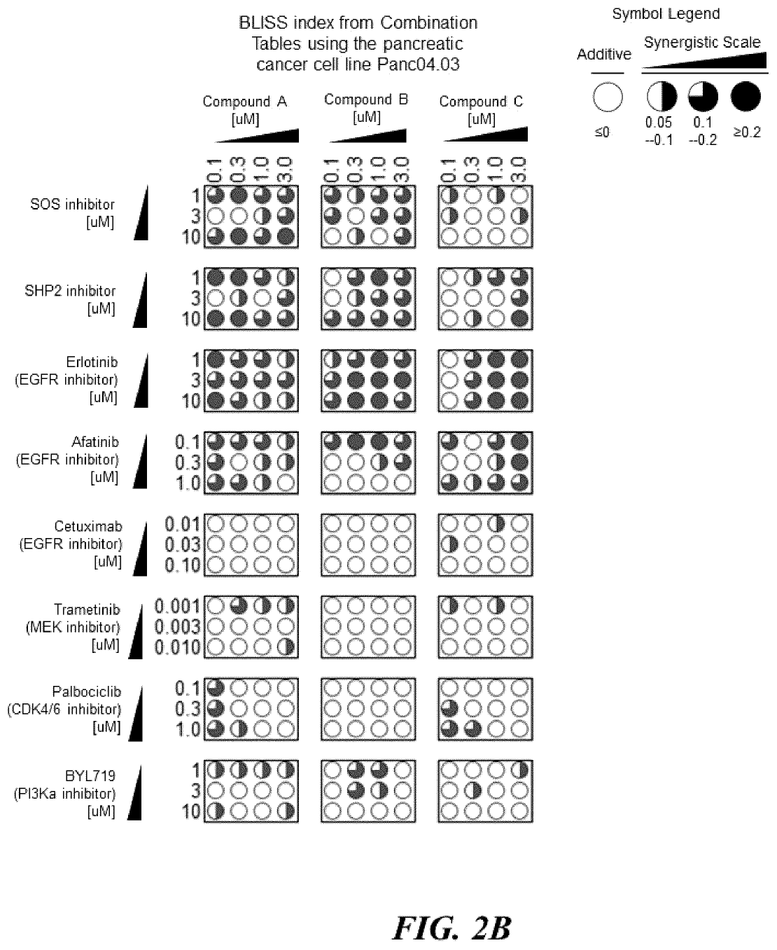

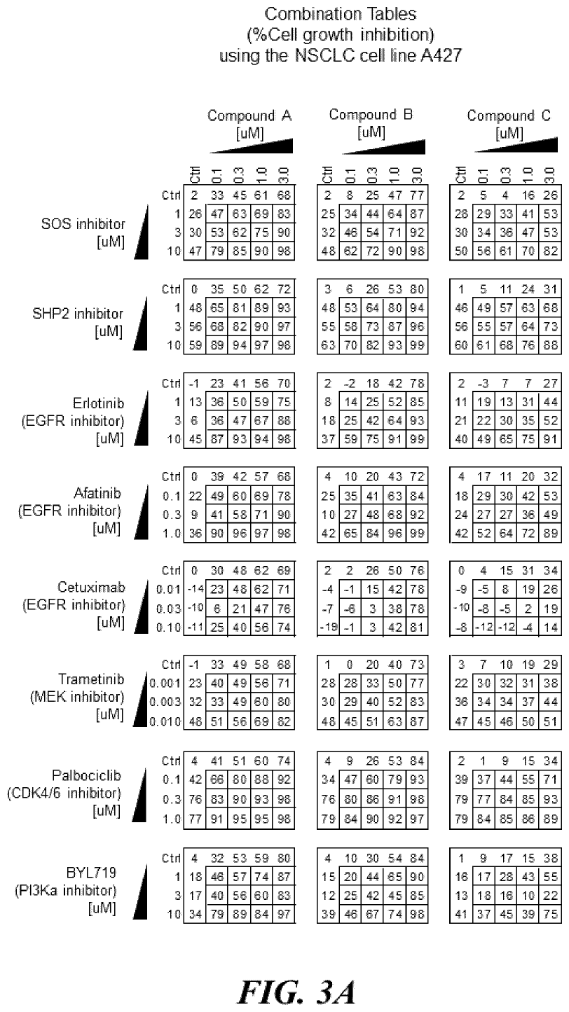

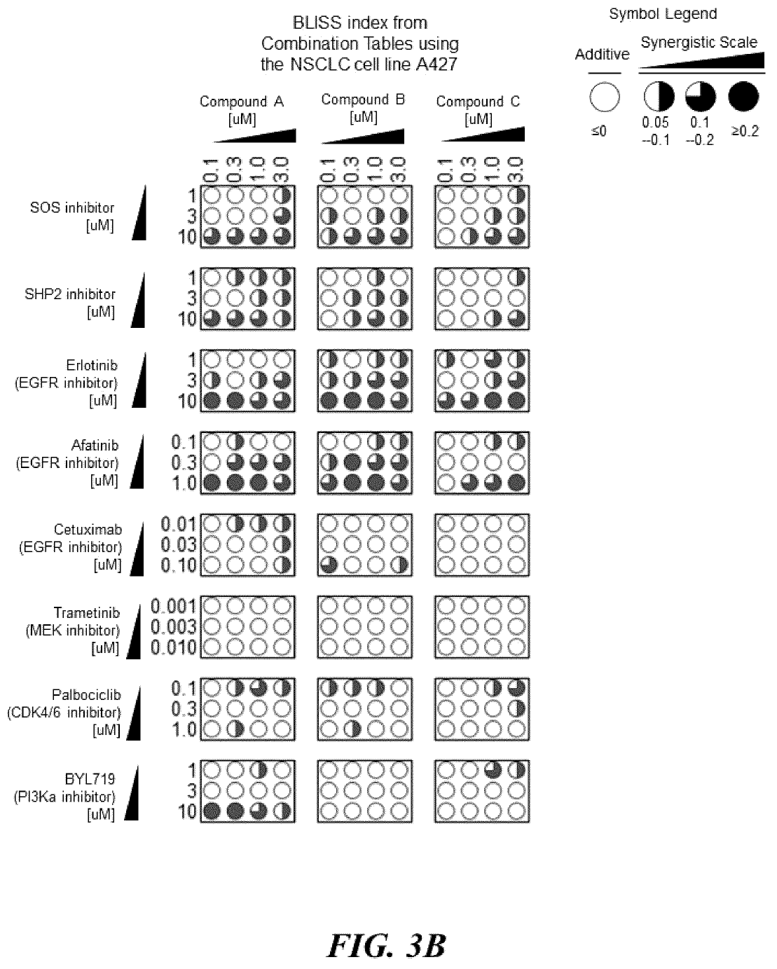

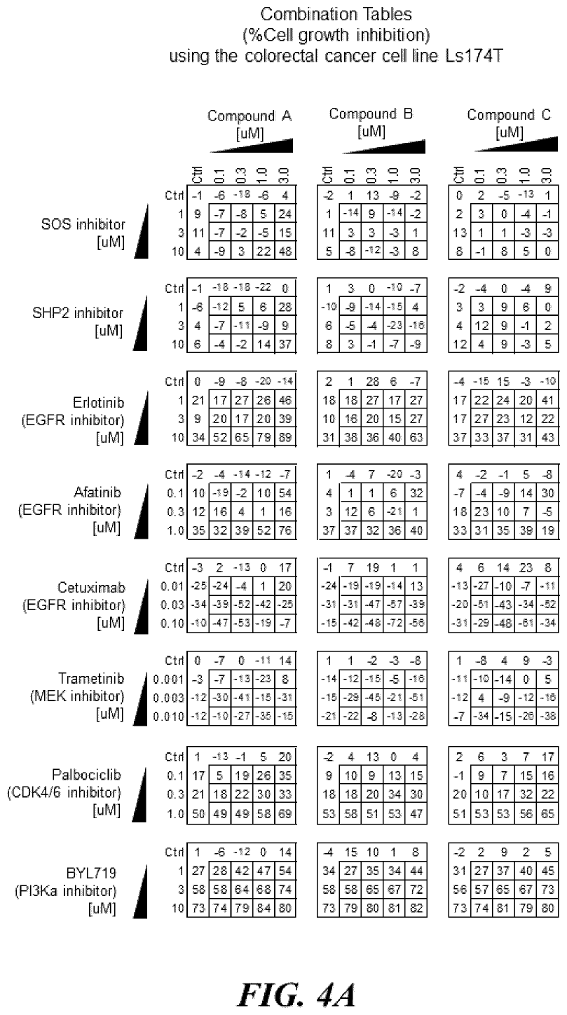

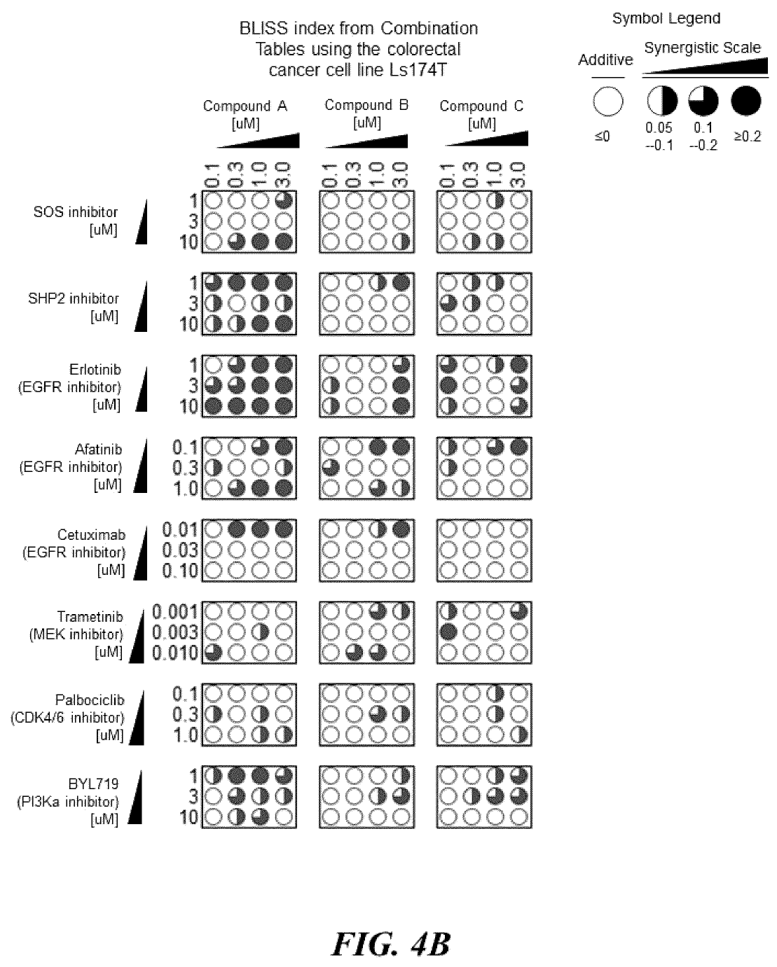

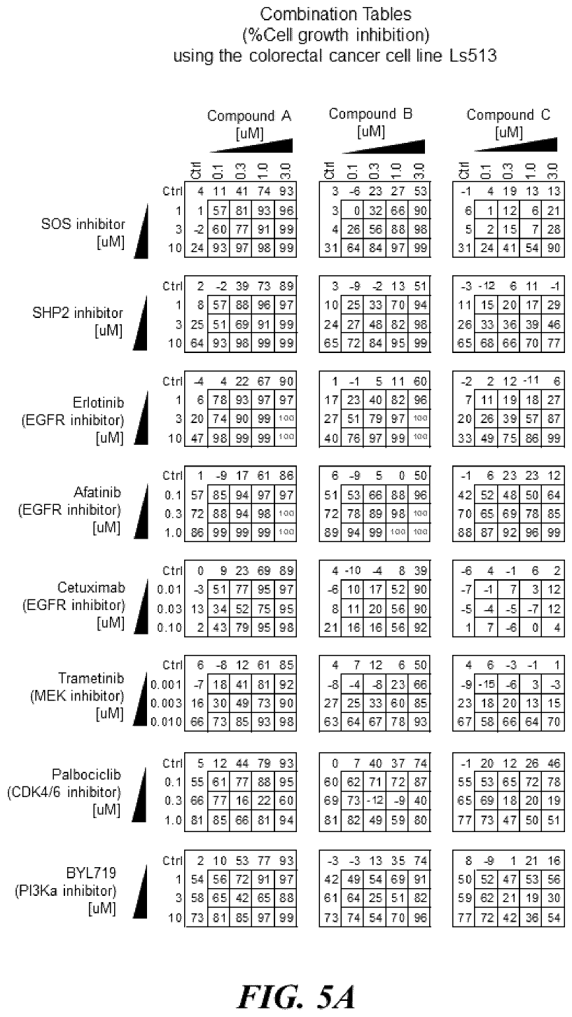

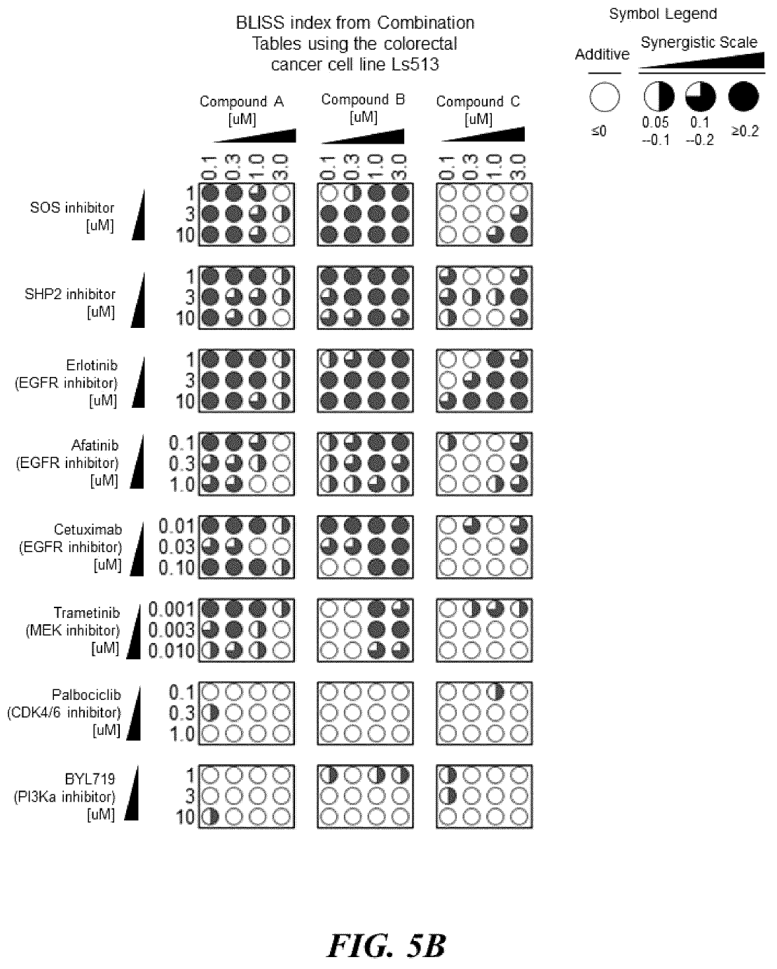

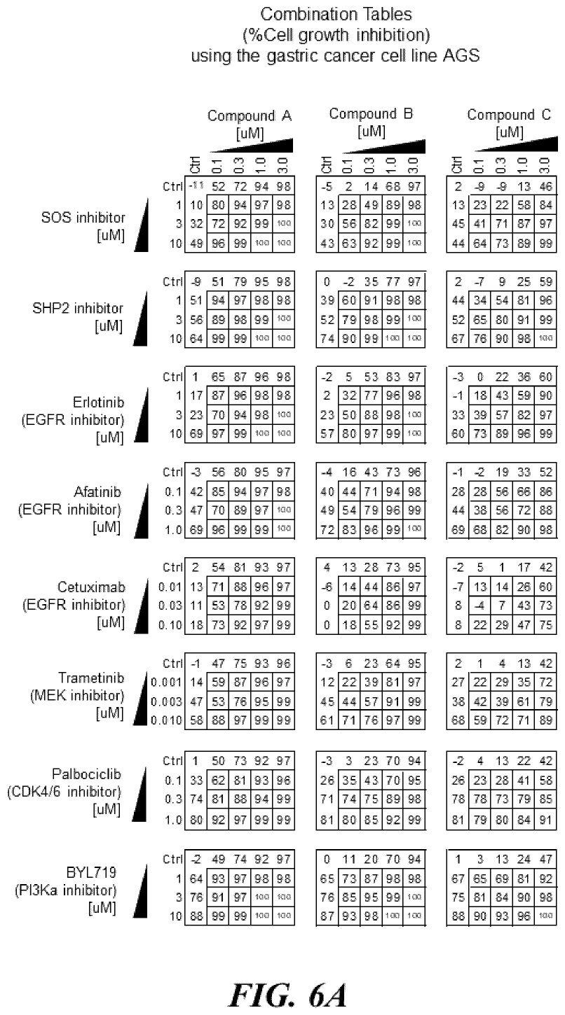

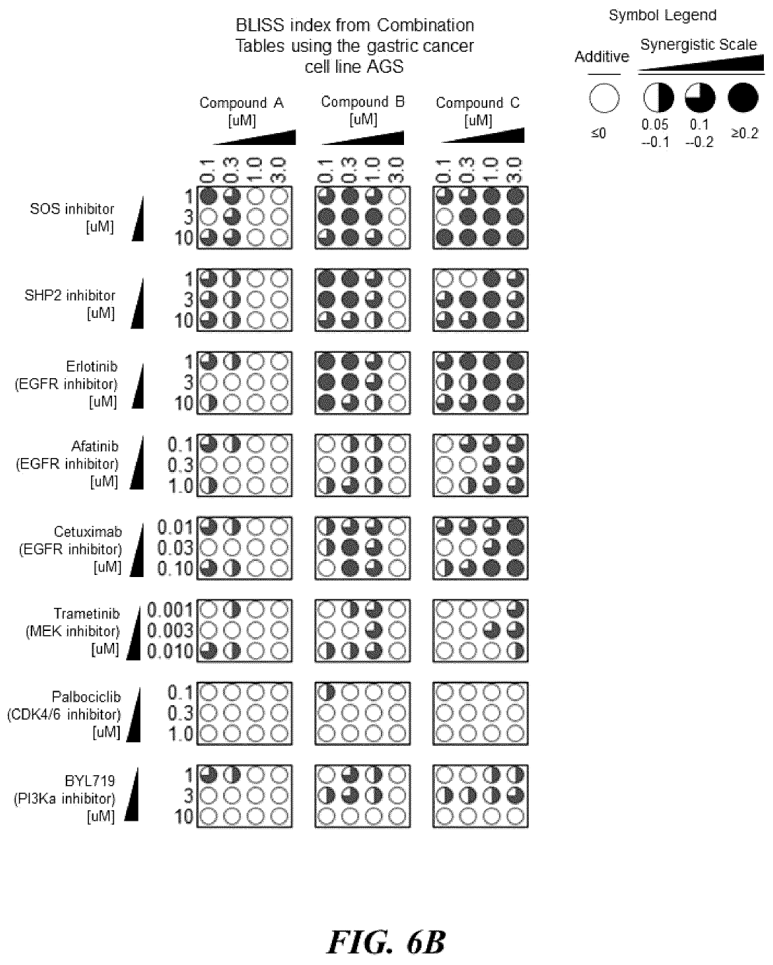

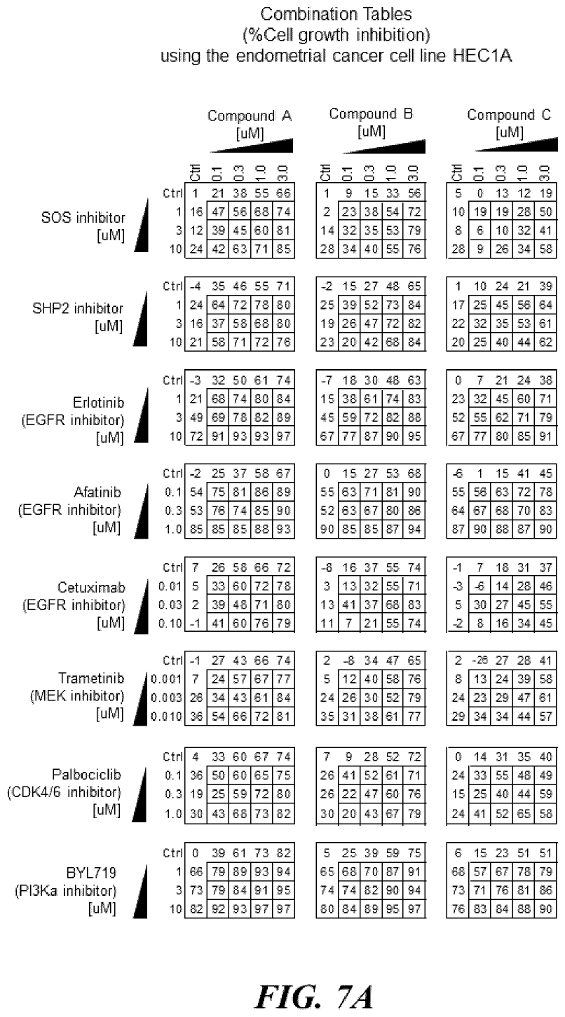

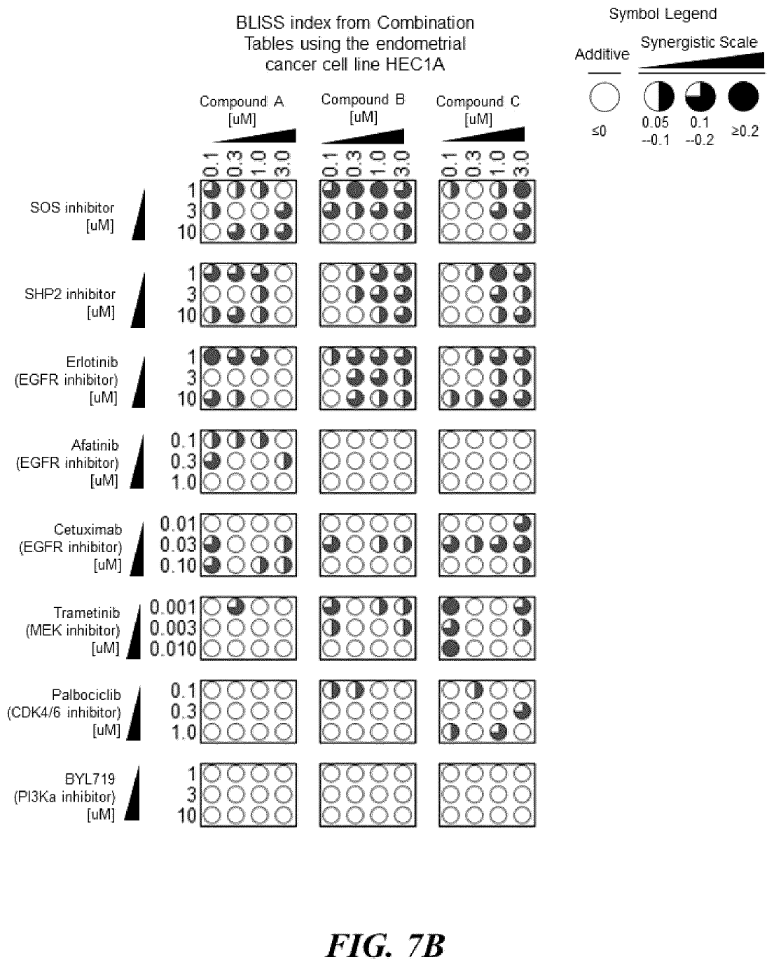

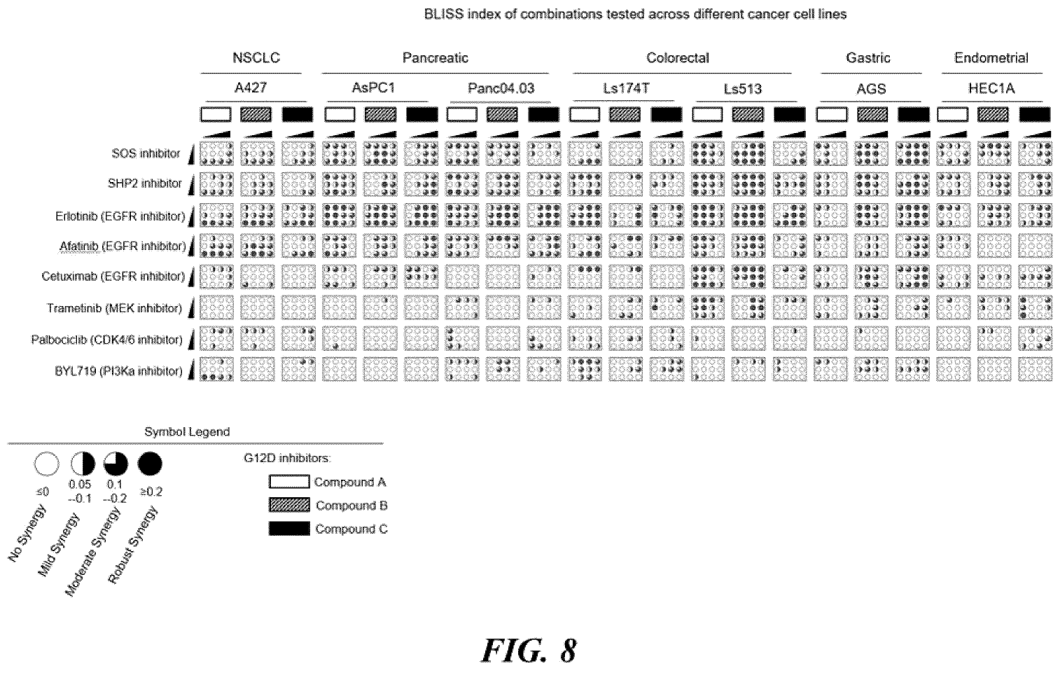

This study explores the inhibition of a variety types of cancer cells (e.g., KRAS G12D driven tumor cells) using a combination of small molecule based Kras12D inhibitor and SOS inhibitor. Cell lines from multiple indications were utilized and include the A427 (a non-small cell lung cancer, “NSCLC”), ASPC1 and Panc04.03 (both pancreatic cancer), Ls174T and Ls513 (both colorectal cancer), AGS (a gastric cancer), and HEClA (an endometrial cancer cell line). All cell lines contain a KRAS G12D mutation. The seven cancer cell lines were cultured in vitro in a 3D proliferation assay where cells are grown in ultra-low attachment surface cell culture plates in RPMI medium containing ˜10% FBS. Cells were incubated with different concentrations of SOS inhibitor (e.g., 10 uM, 3 uM, or 1 uM) and with different concentrations of a Kras12D inhibitors (either Compound A, Compound B, or Compound C, e.g., 0.1 uM, 0.3 uM, 1.0 uM, 3.0 uM) for a period of 5 days. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIGS. 1A, 2A, 3A, 4A, 5A, 6A, and 7A, the degree of enhanced cell growth inhibition is displayed for Kras12D inhibitors alone, SOS inhibitor alone, or in combination with each other respectively in table format with concentrations of each in micromolar. The degree of synergy for the combination across cell lines was calculated by using the Bliss independence model as described herein. The degree of excess over Bliss is the difference in cell growth inhibition between the experimentally observed and the predicted inhibition at various compound concentrations whereby values >0 are indicative of more than additive effects and are instead synergistic. The greater the departure from Bliss indicates more profound synergy: ≤0 indicates no synergism, 0.05-0.1 indicates mild synergy, 0.1-0.2 indicates moderate synergy, and >0.2 indicates robust synergy. In FIGS. 1B, 2B, 3B, 4B, 5B, 6B, and 7B, the assessment of the combinatorial effect using the Bliss model is displayed for indicated cell lines for each Kras12D inhibitor (Compound A, Compound B, or Compound C, having the structure of Formula CA, CJ′, or CF′ disclosed herein) for each concentration tested (displayed in micromolar). Kras12D inhibitors in combination with a SOS inhibitor revealed synergistic cell growth inhibition across a range of concentrations tested (see FIG. 8). For certain cancers including pancreatic, colorectal, and gastric cancers, robust synergy was observed, as assessed in accordance to the Bliss independence model exemplified herein. SOS inhibitor as single agent elicited no more than 50% cell growth inhibition across its concentration range (up to 10 uM) across the cell line panel. Kras12D inhibitor (Compound A, Compound B, or Compound C) as single agent elicited up to complete growth inhibition depending on the cell line tested. The combination however boosted the response and lowered the concentration necessary for the Kras12D inhibitor to achieve 50% growth inhibition (IC50) across the majority of cell lines. The combination lowered the IC50 for all three Kras12D inhibitors up to 10 folds (e.g. A427 and ASPC1).

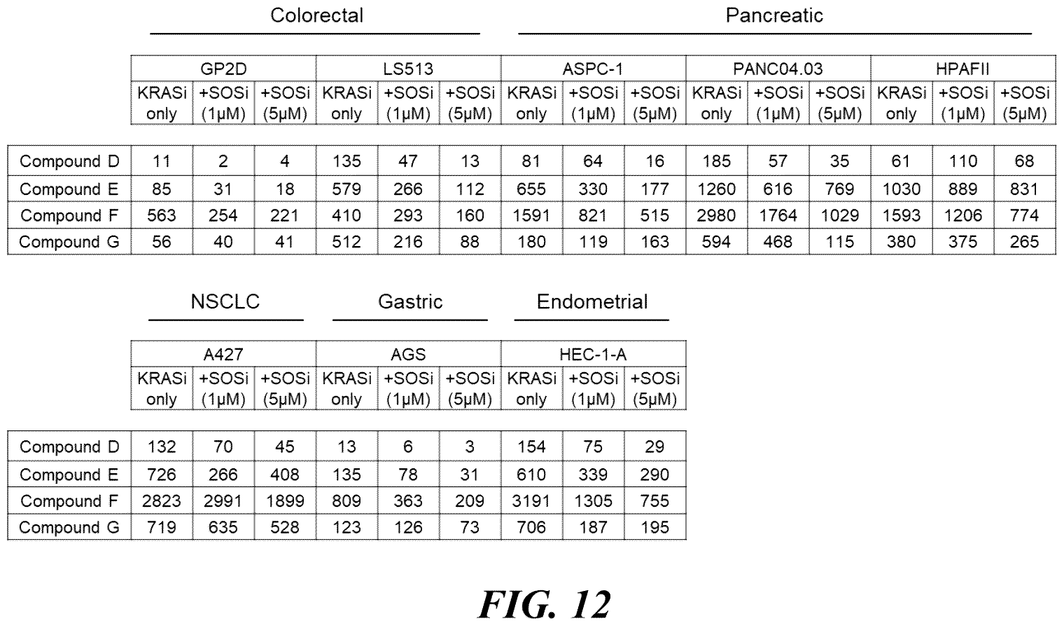

Additional Kras12D inhibitors (Compound D, Compound E, Compound F, and Compound G, having structures of Formula CA, CJ′, or CF′ disclosed herein) exhibited synergistic cell growth inhibition in combination with a SOS inhibitor in a concentration dependent manner (see FIG. 12). Such synergistic inhibition was observed across multiple cancer cell lines, including colorectal, pancreatic, lung (e.g., NSCLC), gastric, and endometrial cancers. When SOS inhibitor was tested alone, no significant cell growth inhibition on these tested cell lines was observed. In addition, Kras12D inhibitor (including Compound E) in combination with a SOS inhibitor of Formula CH yielded at least a 3-, 4-, or 5-fold increase in reducing Kras signaling, as evidenced by a reduction in steady state level of Kras-GTP present in cancer cells lines, such as colorectal (e.g., LS513) and pancreatic (e.g., AsPC1) cell lines.

### Example 13: Small-Molecule Based Kras G12D Inhibitor, with SHP2 Inhibitor

This study explores the inhibition of a variety types of cancer cells (e.g., KRAS G12D driven tumor cells) using a combination of small molecule based Kras12D inhibitor and SHP2 inhibitor. Cell lines from multiple indications were utilized and include the A427 (a non-small cell lung cancer, “NSCLC”), ASPC1 and Panc04.03 (both pancreatic cancer), Ls174T and Ls513 (both colorectal cancer), AGS (a gastric cancer), and HEClA (an endometrial cancer cell line). All cell lines contain a KRAS G12D mutation. The seven cancer cell lines were cultured in vitro in a 3D proliferation assay where cells are grown in ultra-low attachment surface cell culture plates in RPMI medium containing 10% FBS. Cells were incubated with different concentrations of SHP2 inhibitor (e.g., 10 uM, 3 uM, or 1 uM) and with different concentrations of a Kras12D inhibitors (either Compound A, Compound B, or Compound C, e.g., 0.1 uM, 0.3 uM, 1.0 uM, 3.0 uM) for a period of 5 days. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells, as described above. In FIGS. 1A, 2A, 3A, 4A, 5A, 6A, and 7A, the degree of enhanced cell growth inhibition is displayed for Kras12D inhibitors alone, SHP2 inhibitor alone, or in combination with each other respectively in table format with concentrations of each in micromolar. The degree of synergy for the combination across cell lines was calculated by using the Bliss independence model as described in the preceding examples. In FIGS. 1B, 2B, 3B, 4B, 5B, 6B, and 7B, the assessment of the combinatorial effect using the Bliss model is displayed for indicated cell lines for each Kras12D inhibitor (Compound A, Compound B, or Compound C) for each concentration tested (displayed in micromolar). Kras12D inhibitors in combination with a SHP2 inhibitor revealed synergistic cell growth inhibition across a range of concentrations tested (see FIG. 8). For certain cancers including pancreatic, colorectal, and gastric cancers, robust synergy was observed as assessed in accordance to the Bliss independence model exemplified herein. SHP2 inhibitor as single agent elicited no more than about 75% cell growth inhibition across its concentration range (up to 10 uM) across the cell line panel. On average, the maximal cell growth inhibition the SHP2 inhibitor as a single agent elicited was only about 36%. Kras12D inhibitor (Compound A, Compound B, or Compound C) as single agent elicited up to complete growth inhibition depending on the cell line tested. The combination however boosted the response and lowered the concentration necessary for the Kras12D inhibitor to achieve 50% growth inhibition (IC50) across the majority of cell lines. The combination lowered the IC50 for all three Kras12D inhibitors up to 10 folds (e.g. Ls513 and AGS).

### Example 14: Small-Molecule Based Kras G12D Inhibitor, with EGFR Inhibitor

The study explores the inhibition of a variety types of cancer cells (e.g., KRAS G12D driven tumor cells) using a combination of small molecule based Kras12D inhibitor and EGFR inhibitor. Cell lines from multiple indications were utilized and include the A427 (a non-small cell lung cancer, “NSCLC”), ASPC1 and Panc04.03 (both pancreatic cancer), Ls174T and Ls513 (both colorectal cancer), AGS (a gastric cancer), and HEClA (an endometrial cancer cell line). All cell lines contain a KRAS G12D mutation. The seven cancer cell lines were cultured in vitro in a 3D proliferation assay where cells are grown in ultra-low attachment surface cell culture plates in RPMI medium containing 10% FBS. Cells were then incubated with a concentration range of erlotinib (e.g., 1, 3, and 10 uM), afatinib (e.g., 0.1, 0.3, and 1 uM), or cetuximab (e.g., 0.01, 0.03, and 0.1 uM), respectively, for a period of about 5 days. The concentration of the respective EGFR inhibitors utilized in this cell assay was chosen to be within the range corresponding to achievable exposures and pharmacokinetics of a recommended and clinical relevant dose applied in human. A Kras12D inhibitor (either Compound A, Compound B, or Compound C, e.g., 0.1 uM, 0.3 uM, 1.0 uM, 3.0 uM) was utilized in this combination for a period of 5 days. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIGS. 1A, 2A, 3A, 4A, 5A, 6A, and 7A, the degree of enhanced cell growth inhibition is displayed for Kras12D inhibitors alone, EGFR inhibitor alone, or in combination with each other respectively in table format with concentrations of each in micromolar. The degree of synergy for the combination across cell lines was calculated by using the Bliss independence model, as described above. The degree of excess over Bliss is the difference in cell growth inhibition between the experimentally observed and the predicted inhibition at various compound concentrations as described in the preceding examples. In FIGS. 1B, 2B, 3B, 4B, 5B, 6B, and 7B, the assessment of the combinatorial effect using the Bliss model is displayed for indicated cell lines for each Kras12D inhibitor (Compound A, Compound B, or Compound C) for each concentration tested (displayed in micromolar). Kras12D inhibitors in combination with an EGFR inhibitor revealed synergistic cell growth inhibition across a range of concentrations tested (see FIG. 8). For certain cancers including NSCLC, pancreatic, colorectal, and gastric cancers, robust synergy was observed) as assessed in accordance to the Bliss independence model exemplified herein. Erlotinib as a single agent elicited no more than about 72% cell growth inhibition across its concentration range (up to 10 uM) across the cell line panel. On average, the maximal cell growth inhibition response of erlotinib as a single agent was under about 50%. Afatinib as a single agent elicited no more than about 86% cell growth inhibition across its concentration range (up to 1 uM) across the cell line panel. On average, the maximal cell growth inhibition response of erlotinib as a single agent was under about 40%. Cetuximab as a single agent elicited no more than 18% cell growth inhibition across its concentration range (up to 0.1 uM) across the cell line panel. On average, the maximal cell growth inhibition response of erlotinib as a single agent was under about 20%. Kras12D inhibitor (Compound A, Compound B, or Compound C) as single agent elicited up to complete growth inhibition depending on the cell line tested. Together, the EGFR inhibitor combinations as a class boosted the response and lowered the concentration necessary for the Kras12D inhibitor to achieve 50% growth inhibition (IC50) across the majority of cell lines. The combination lowered the IC50 for all three Kras12D inhibitors up to or greater than 10 folds (e.g. Ls513 and AGS).

### Example 15: Small-Molecule Based Kras G12D Inhibitor, with MEK Inhibitor

The study explores the inhibition of a variety types of cancer cells (e.g., KRAS G12D driven tumor cells) using a combination of small molecule based Kras12D inhibitor and MEK inhibitor. Cell lines from multiple indications were utilized and include the A427 (a non-small cell lung cancer, “NSCLC”), ASPC1 and Panc04.03 (both pancreatic cancer), Ls174T and Ls513 (both colorectal cancer), AGS (a gastric cancer), and HEClA (an endometrial cancer cell line). All cell lines contain a KRAS G12D mutation. The seven cancer cell lines were cultured in vitro in a 3D proliferation assay where cells are grown in ultra-low attachment surface cell culture plates in RPMI medium containing 10% FBS. Cells were incubated with different concentrations of MEK inhibitor (e.g., 0.001 uM, 0.003 uM, 0.01 uM) and with different concentrations of a Kras12D inhibitors (either Compound A, Compound B, or Compound C, e.g., 0.1 uM, 0.3 uM, 1.0 uM, 3.0 uM) for a period of 5 days. The concentrations of the MEK inhibitors utilized in this cell assay were chosen to be within the range corresponding to achievable exposures and pharmacokinetics of a recommended and clinical relevant dose applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIGS. 1A, 2A, 3A, 4A, 5A, 6A, and 7A, the degree of enhanced cell growth inhibition is displayed for Kras12D inhibitors alone, MEK inhibitor alone, or in combination with each other respectively in table format with concentrations of each in micromolar. The degree of synergy for the combination across cell lines was calculated by using the Bliss independence model, as described above. The degree of excess over Bliss is the difference in cell growth inhibition between the experimentally observed and the predicted inhibition at various compound concentrations as described in the preceding examples. In FIGS. 1B, 2B, 3B, 4B, 5B, 6B, and 7B, the assessment of the combinatorial effect using the Bliss model is displayed for indicated cell lines for each Kras12D inhibitor (Compound A, Compound B, or Compound C) for each concentration tested (displayed in micromolar). Kras12D inhibitors in combination with a MEK inhibitor revealed synergistic cell growth inhibition across a range of concentrations tested in some but not all cancer cell lines. Robust synergy was observed in colorectal cancer, as assessed in accordance to the Bliss independence model exemplified herein (see FIG. 8). MEK inhibitor as single agent elicited no more than 68% cell growth inhibition across its concentration range (up to 0.01 uM) across the cell line panel. On average, the maximal cell growth inhibition the MEK inhibitor as a single agent elicited was only about 42%. Kras12D inhibitor (Compound A, Compound B, or Compound C) as single agent elicited up to complete growth inhibition depending on the cell line tested. The combination however boosted the response and lowered the concentration necessary for the Kras12D inhibitor to achieve 50% growth inhibition (IC50) across the majority of cell lines. The combination lowered the IC50 for all three Kras12D inhibitors up to and greater than 3-folds (e.g. Ls513, Panc04.03, and AGS).

### Example 16: Small-Molecule Based Kras G12D Inhibitor, with CDK4/6 Inhibitor

This study explores the inhibition of a variety types of cancer cells (e.g., KRAS G12D driven tumor cells) using a combination of small molecule based Kras12D inhibitor and CDK4/6 inhibitor. Cell lines from multiple indications were utilized and include the A427 (a non-small cell lung cancer, “NSCLC”), ASPC1 and Panc04.03 (both pancreatic cancer), Ls174T and Ls513 (both colorectal cancer), AGS (a gastric cancer), and HEClA (an endometrial cancer cell line). All cell lines contain a KRAS G12D mutation. The seven cancer cell lines were cultured in vitro in a 3D proliferation assay where cells are grown in ultra-low attachment surface cell culture plates in RPMI medium containing 10% FBS. Cells were incubated with different concentrations of CDK4/6 inhibitor (palbociclib, at 0.1 uM, 0.3 uM, 1.0 uM) and with different concentrations of a Kras12D inhibitors (either Compound A, Compound B, or Compound C, e.g., 0.1 uM, 0.3 uM, 1.0 uM, 3.0 uM) for a period of 5 days. The concentrations of the CDK4/6 inhibitor utilized in this cell assay were chosen to be within the range corresponding to achievable exposures and pharmacokinetics of a recommended and clinical relevant dose applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIGS. 1A, 2A, 3A, 4A, 5A, 6A, and 7A, the degree of enhanced cell growth inhibition is displayed for Kras12D inhibitors alone, CDK4/6 inhibitor alone, or in combination with each other respectively in table format with concentrations of each in micromolar. The degree of synergy for the combination across cell lines was calculated by using the Bliss independence model, as described above. The degree of excess over Bliss is the difference in cell growth inhibition between the experimentally observed and the predicted inhibition at various compound concentrations as described in the preceding examples. In FIGS. 1B, 2B, 3B, 4B, 5B, 6B, and 7B, the assessment of the combinatorial effect using the Bliss model is displayed for indicated cell lines for each Kras12D inhibitor (Compound A, Compound B, or Compound C) for each concentration tested (displayed in micromolar). Kras12D inhibitors in combination with a CDK4/6 inhibitor revealed synergistic cell growth inhibition across a range of concentrations tested in some but not all cancer cell lines. Mild synergy was observed in lung, pancreatic and colorectal cancer, as assessed in accordance to the Bliss independence model exemplified herein (see FIG. 8). CDK4/6 inhibitor as single agent elicited no more than 81% cell growth inhibition across its concentration range (up to 1 uM) across the cell line panel. On average, the maximal cell growth inhibition the CDK4/6 inhibitor as a single agent elicited was only about 48%. Kras12D inhibitor (Compound A, Compound B, or Compound C) as single agent elicited up to complete growth inhibition depending on the cell line tested. The combination however boosted the response and lowered the concentration necessary for the Kras12D inhibitor to achieve 50% growth inhibition (IC50) across the majority of cell lines. The combination lowered the IC50 for all three Kras12D inhibitors up to and greater than 3-fold (e.g. A427, ASPC1, and Panc04.03).

### Example 17: Small-Molecule Based Kras G12D Inhibitor, with PI3K Inhibitor

This study explores the inhibition of a variety types of cancer cells (e.g., KRAS G12D driven tumor cells) using a combination of small molecule based Kras12D inhibitor and PI3K-alpha (PI3Ka) inhibitor. Cell lines from multiple indications were utilized and include the A427 (a non-small cell lung cancer, “NSCLC”), ASPC1 and Panc04.03 (both pancreatic cancer), Ls174T and Ls513 (both colorectal cancer), AGS (a gastric cancer), and HEClA (an endometrial cancer cell line). All cell lines contain a KRAS G12D mutation. The seven cancer cell lines were cultured in vitro in a 3D proliferation assay where cells are grown in ultra-low attachment surface cell culture plates in RPMI medium containing 10% FBS. Cells were incubated with different concentrations of PI3Ka inhibitor (BYL719, at 1 uM, 3 uM, 10 uM) and with different concentrations of a Kras12D inhibitors (either Compound A, Compound B, or Compound C, e.g., 0.1 uM, 0.3 uM, 1.0 uM, 3.0 uM) for a period of 5 days. The concentrations of the PI3Ka inhibitor utilized in this cell assay were chosen to be within the range corresponding to achievable exposures and pharmacokinetics of a recommended and clinical relevant dose applied in human. The amount of cell growth inhibition was calculated and reported whereby a 100% inhibition is equivalent to complete blockade of growth and 0% is no effect and <100% reflects a partial growth-inhibitory effect relative to vehicle (DMSO)-treated control (Ctrl) cells. In FIGS. 1A, 2A, 3A, 4A, 5A, 6A, and 7A, the degree of enhanced cell growth inhibition is displayed for Kras12D inhibitors alone, PI3Ka inhibitor alone, or in combination with each other respectively in table format with concentrations of each in micromolar. The degree of synergy for the combination across cell lines was calculated by using the Bliss independence model, as described above. The degree of excess over Bliss is the difference in cell growth inhibition between the experimentally observed and the predicted inhibition at various compound concentrations as described in the preceding examples. In FIGS. 1B, 2B, 3B, 4B, 5B, 6B, and 7B, the assessment of the combinatorial effect using the Bliss model is displayed for indicated cell lines for each Kras12D inhibitor (Compound A, Compound B, or Compound C) for each concentration tested (displayed in micromolar). Kras12D inhibitors in combination with a PI3Ka inhibitor revealed synergistic cell growth inhibition across a range of concentrations tested in certain cancer cell lines. Mild to robust synergy was observed in colorectal and gastric cancer, and particularly cancers (e.g., colorectal and/or gastric) comprising genetic aberration of PI3Ka gene (e.g., AGS, Ls174T), as assessed in accordance to the Bliss independence model exemplified herein (see FIG. 8). PI3Ka inhibitor as single agent elicited no more than 88% cell growth inhibition across its concentration range (up to 10 uM) across the cell line panel. On average, the maximal cell growth inhibition the PI3Ka inhibitor as a single agent elicited was only about 58%. Kras12D inhibitor (Compound A, Compound B, or Compound C) as single agent elicited up to complete growth inhibition depending on the cell line tested. The combination however boosted the response and lowered the concentration necessary for the Kras12D inhibitor to achieve 50% growth inhibition (IC50) across the majority of cell lines. The combination lowered the IC50 for all three Kras12D inhibitors up to and greater than 3-fold (e.g. HEClA, Ls174T, and AGS). In particular the colorectal cancer cell line Ls174T had a robust synergistic response to the combination. The Kras12D inhibitors as single agents in this cell line had minimal activity with maximal response elicited up to about 17%. As a combination, PI3Ka inhibitor synergistically potentiated the response of the Kras12D inhibitor allowing for up to 84% maximal cell growth inhibition.

### Example 18: In Vivo Ras Inhibition in Xenograft Models

The in vivo reduction in Ras signaling output by the inhibitor of the present disclosure can be determined in a mouse tumor xenograft model. Tumor xenografts can be established by administration of tumor cells with K-Ras G12D mutation into mice. Mice xenografts of diseased cells derived from a particular type of tumor or cancer can be used as model to assess (or predict) efficacy of the inhibitor of the present disclosure as a medicament for a subject having or is suspected of having the same type of tumor or cancer.

Xenograft Study:

Female 6- to 8-week-old athymic BALB/c nude (NCr) nu/nu mice are used for xenografts. The tumor cells (e.g., approximately 5×10⁶) are harvested on the day of use and injected in growth-factor-reduced Matrigel/PBS (e.g., 50% final concentration in 100 μl). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L x w²)/2 in which L and w refer to the length and width tumor diameter, respectively. Percent tumor volume change can be calculated using the following formula: (V_{final}−Vᵢₙᵢₜᵢₐₗ)/Vᵢₙᵢₜᵢₐₗ×100. Percent of tumor growth inhibition (% TGI) can be calculated using the following formula: % TGI=100×(1−(average V_{final}−Vᵢₙᵢₜᵢₐₗ of treatment group)/(average V_{final}−Vᵢₙᵢₜᵢₐₗ of control group). When tumors reach a threshold average size (e.g., approximately 200-400 mm³). mice are randomized into 3-10 mice (e.g., 8-10 mice) per group and are treated with vehicle (e.g., 100% Labrasol®) or the inhibitor of the present disclosure using, for example, a daily schedule by oral gavage. Results can be expressed as mean and standard deviation of the mean.

Lung Cancer/Tumor Cell Xenograft Models:

To assess or predict the efficacy of the inhibitor for treating lung cancer (e.g., non-small cell lung cancer (NSCLC)), any of the following cells with K-Ras G12D mutation are administered as aforementioned to establish mice tumor xenografts: A427 and SKLU1. When tumors reach a threshold average size, mice are randomized into 3-10 per group and each group is treated with (1) vehicle, (2) an inhibitor of Kras G12D, (3) an inhibitor of SOS1 (e.g., BAY-293, BI-1701963), or (4) a combination of the inhibitor of Kras G12D and the inhibitor of SOS1 using, for example, a daily schedule by oral gavage. The combination treatment to Group 4 mice is expected to yield a greater extent and/or a longer period of tumor reduction.

Colorectal Cancer/Tumor Cell Xenograft Models:

To assess or predict the efficacy of the inhibitor for treating colorectal cancer, any of the following cells with K-Ras G12D mutation are administered as aforementioned to establish mice tumor xenografts: Ls513, Ls180, Ls147t, SNU-407, and SNU-C2A. When tumors reach a threshold average size, mice are randomized into 3-10 per group and each group is treated with (1) vehicle as a negative control, (2) an inhibitor of Kras G12D, (3) an inhibitor of EGFR (e.g., afatinib, erlotinib, gefitinib, cetuximab), or (4) a combination of the inhibitor of Kras G12D and the inhibitor of EGFR using, for example, a daily schedule by oral gavage. The combination treatment to Group 4 mice is expected to yield a greater extent and/or a longer period of tumor reduction.

Pancreatic Cancer/Tumor Cell Xenograft Models:

To assess (or predict) the efficacy of the inhibitor for treating pancreatic cancer (e.g., pancreatic ductal adenocarcinoma (PDAC)), any of the following cells with K-Ras G12D mutation are administered as aforementioned to establish mice tumor xenografts: ASPC-1, HPAFII, and SW1990. When tumors reach a threshold average size, mice are randomized into 3-10 per group and each group is treated with (1) vehicle as a negative control, (2) an inhibitor of Kras G12D, (3) an inhibitor of EGFR (e.g., erlotinib), MEK (e.g., trametinib), or SOS1 (e.g., BAY-293, BI-1701963), or (4) a combination of the inhibitor of Kras G12D and the inhibitor of EGFR, MEK, or SOS1 using, for example, a daily schedule by oral gavage. Optionally, the mice can be administered with aforementioned treatments in combination with an anti-cancer agents, such as an inhibitor of PARP (e.g., olaparib) or ribonucleotide reductase (RNR) (e.g., gemcitabine). The combination treatment to Group 4 mice is expected to yield a greater extent and/or a longer period of tumor reduction.

### Example 19: In Vivo Ras Inhibition in Transgenic Models

The in vivo reduction in Ras signaling output by the inhibitor of the present disclosure can be determined in a transgenic model. The transgenic model can have one or more somatic mutations, such as K-Ras G12D and/or PIK3CA H1047R (p110-a subunit of PI3K). The transgenic animals can be used as model to assess (or predict) efficacy of the inhibitor of the present disclosure as a medicament for a subject having or is suspected of having the same type of somatic mutation and a resulting phenotype, such as lung cancer.

Lung Cancer Transgenic Model:

Transgenic mice having either (i) K-Ras G12D mutation or (ii) K-Ras G12D and PIK3CA H1047R mutations with an appropriate lung tumor burden (e.g., as ascertained by magnetic resonance imaging (MRI)) are randomized into 3-10 per group and each group is treated with (1) vehicle as a negative control, (2) an inhibitor of Kras G12D, (3) an inhibitor of MEK (e.g., trametinib, AZD6244) or PI3K (e.g., NVP-BEZ235-AN), or (4) a combination of the inhibitor of Kras G12D and the inhibitor of MEK or PI3K using, for example, a daily schedule by oral gavage. The combination treatment to Group 4 mice is expected to yield a greater extent and/or a longer period of lung tumor reduction.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternative s to the embodiments of the disclosure described herein may be employed in practicing the invention. It is intended that the following claims define the scope of the invention and that methods and structures within the scope of these claims and their equivalents be covered thereby.

1. A method of inhibiting proliferation signaling in cancer cells, the method comprising: downregulating, in the cancer cells, expression or activity of: (i) a Kras G12D and (ii) at least one signaling molecule selected from Table 1, wherein the downregulating the expression or activity of (i) and that of (ii) synergistically yields a greater degree of inhibition of proliferation of the cell as compared to downregulating expression or activity of one of: (i) and (ii).

2. The method of claim 1, wherein the step of downregulating comprises contacting the cancer cells with (a) a Kras G12D inhibitor and (b) at least one inhibitor of the signaling molecule, wherein contacting the cancer cells with (a) occurs prior to, concurrently with, or subsequent to contacting the cancer cells with (b), to effect a reduced proliferation of the cancer cells, wherein the reduced proliferation of the cancer cells by application of (a) and (b) is characterized by a synergistic value of at least about 0.05 as ascertained by Bliss independent criterion.

3. The method of claim 2, wherein the synergistic value is ascertained by Bliss independent criterion in accordance to the formula:
Y_{AB,O}−Y_{AB,P}
wherein:
Y_{AB,O} is observed percentage growth inhibition of the cancer cells by the application of (a) and (b) comprising (a) at dose A and (b) at dose B; and
Y_{AB,P} is predicted percentage growth inhibition of the cancer cells by the application of (a) and (b) comprising (a) at the dose A, and (b) at the dose B, wherein Y_{AB,P}=Y_{A}+Y_{B}−Y_{A}Y_{B},
wherein further:
Y_{A} is observed percentage growth inhibition of the cancer cells by (a) alone at the dose A;
Y_{B} is observed percentage growth inhibition of the cancer cells by (b) alone at the dose B; and
Y_{A}Y_{B} is product of Y_{A} and Y_{B}.

4. The method of claim 2, wherein the synergistic value is at least about 0.1.

5. The method of claim 2, wherein the synergistic value is at least about 0.2.

6. The method of claim 1, wherein the cancer cells are derived from one or more members selected from the group consisting of non-small cell lung cancer, pancreatic cancer, colorectal cancer, gastric cancer, and endometrial cancer.

7. The method of claim 1, wherein the cancer cells are derived from colorectal cancer or gastric cancer.

8. The method of claim 1, wherein the signaling molecule is selected from the group consisting of SOS, SHP2, EGFR, MEK, CDK4/6, and PI3Ka.

9. The method of claim 1, wherein the signaling molecule is SHP2.

10. The method of claim 1, wherein the signaling molecule is SOS.

11. The method of claim 1, wherein the signaling molecules is EGFR.

12. The method of claim 1, wherein the cancer cells are contacted with at least two inhibitors, one of which is an inhibitor of SOS, and another is an inhibitor of EGFR.

13. A method of inhibiting proliferation signaling in cancer cells, the method comprising: downregulating, in the cancer cells, expression or activity of: (i) a Kras G12D and (ii) a signaling molecule selected from the group consisting of SOS, SHP2, and EGFR, wherein the cancer cells are derived from one or more members selected from the group consisting of non-small cell lung cancer, pancreatic cancer, colorectal cancer, gastric cancer, and endometrial cancer.

14. The method of claim 13, wherein the step of downregulating comprises contacting the cancer cells with (a) a Kras G12D inhibitor and (b) at least one inhibitor of the signaling molecule selected from the group consisting of SOS, SHP2, and EGFR, wherein the contacting the cancer cells with (a) occurs prior to, concurrently with, or subsequent to contacting the cancer cells with (b), to effect a reduced proliferation of the cancer cells in the subject.

15. The method of claim 14, wherein the reduced proliferation of the cancer cells by an application of (a) and (b) is characterized by a synergistic value of at least about 0.05 as ascertained by Bliss independent criterion.

16-20. (canceled)

21. A method of treating colorectal cancer or gastric cancer in a subject, wherein the subject exhibits a genetic aberration in a PI3K gene, the method comprising administering to the subject (a) a Kras G12D inhibitor and (b) at least one inhibitor of PI3K, wherein (a) is administered prior to, concurrently with, or subsequent to administering (b), such that application of (a) and (b) effects reduced proliferation of colorectal cancer cells or gastric cancer cells in the subject.

22. (canceled)

23. The method of claim 21, wherein the application exhibits a synergistic effect in reducing the proliferation of the colorectal cancer cells or the gastric cancer cells in the subject, as compared to either of (a) alone or (b) alone.

24. The method of claim 21, wherein the reduced proliferation of the colorectal cancer cells or the gastric cancer cells by the application of (a) and (b) is characterized by a synergistic value of at least about 0.05 as ascertained by Bliss independent criterion.

25-64. (canceled)

65. The method of claim 1, wherein application of (a) and (b) yields a reduced proliferation signaling characterized by reduced Ras signaling output in a cell.

66. The method of claim 65, wherein the reduction in Ras signaling output is evidenced by one or more members selected from the group consisting of (i) an increase in steady state level of GDP-bound Ras protein; (ii) a reduction of phosphorylated AKT^{s473}, (iii) a reduction of phosphorylated ERK^{T202/y204}, (iv) a reduction of phosphorylated S6^{S235/236}, and (v) inhibition of cell growth.

67-109. (canceled)
